(19) United States
(12) Patent Application Publication
(10) Pub. No.: US 2020/0239553 A1
(43) Pub. Date: Jul. 30, 2020

(54) BCL-XL Inhibitory Compounds and Antibody Drug Conjugates Including the Same

(71) Applicant: AbbVie Inc., North Chicago, IL (US)

(72) Inventors: Scott L. Ackler, Gurnee, IL (US); Nathan B. Bennett, Gurnee, IL (US); Erwin R. Boghaert, Pleasant Prairie, WI (US); Steve C. Cullen, Lake Villa, IL (US); George Doherty, Libertyville, IL (US); Robin R. Frey, Libertyville, IL (US); Anthony R. Haight, Wadsworth, IL (US); Andrew S. Judd, Grayslake, IL (US); Aaron R. Kunzer, Arlington Heights, IL (US); Xiaoqiang Shen, Lincolnshire, IL (US); Xiaohong Song, Grayslake, IL (US); Andrew J. Souers, Libertyville, IL (US); Gerard M. Sullivan, Libertyville, IL (US); Zhi-Fu Tao, Vernon Hills, IL (US); Xilu Wang, Libertyville, IL (US); Dennie S. Welch, Gurnee, IL (US); Michael D. Wendt, Vernon Hills, IL (US)

(73) Assignee: AbbVie Inc., North Chicago, IL (US)

(21) Appl. No.: 16/675,784

(22) Filed: Nov. 6, 2019

Related U.S. Application Data
(63) Continuation of application No. 16/358,963, filed on Mar. 20, 2019, now abandoned.
Application No. 16/358,963 is a continuation of application No. 14/963,510, filed on Dec. 9, 2015, now abandoned.
(60) Provisional application No. 62/089,794, filed on Dec. 9, 2014.

(51) Int. Cl. C07K 16/18; C07D 417/14; C07D 487/04; C07D 513/04; A61K 47/68

(52) U.S. Cl. CPC C07K 16/18; C07D 417/14; C07K 2317/73; C07D 513/04; A61K 47/6803; C07D 487/04

(57) ABSTRACT

Small molecule Bcl-xL inhibitors and Antibody Drug Conjugates (ADCs) comprising small molecule Bcl-xL inhibitors are disclosed herein. The Bcl-xL inhibitors and ADCs of the disclosure are useful for, among other things, inhibiting anti-apoptotic Bcl-xL proteins as a therapeutic approach towards the treatment of diseases that involve a dysregulated apoptosis pathway.

### 1. FIELD

The present disclosure pertains to compounds that inhibit the activity of Bcl-xL anti-apoptotic proteins, antibody drug conjugates comprising these inhibitors, methods useful for synthesizing these inhibitors and antibody drug conjugates, compositions comprising the inhibitors, and antibody drug conjugates, and methods of treating diseases in which anti-apoptotic Bcl-xL proteins are expressed.

### 2. BACKGROUND

Apoptosis is recognized as an essential biological process for tissue homeostasis of all living species. In mammals in particular, it has been shown to regulate early embryonic development. Later in life, cell death is a default mechanism by which potentially dangerous cells (e.g., cells carrying cancerous defects) are removed. Several apoptotic pathways have been uncovered, and one of the most important involves the Bcl-2 family of proteins, which are key regulators of the mitochondrial (also called “intrinsic”) pathway of apoptosis. See, Danial & Korsmeyer, 2004, Cell 116:205-219.

Dysregulated apoptotic pathways have been implicated in the pathology of many significant diseases such as neurodegenerative conditions (up-regulated apoptosis), such as for example, Alzheimer's disease; and proliferative diseases (down-regulated apoptosis) such as for example, cancer, autoimmune diseases and pro-thrombotic conditions.

In one aspect, the implication that down-regulated apoptosis (and more particularly the Bcl-2 family of proteins) is involved in the onset of cancerous malignancy has revealed a novel way of targeting this still elusive disease. Research has shown, for example, the anti-apoptotic proteins, Bcl-2 and Bcl-xL, are over-expressed in many cancer cell types. See, Zhang, 2002, Nature Reviews/Drug Discovery 1:101; Kirkin et al., 2004, Biochimica Biophysica Acta 1644:229-249; and Amundson et al., 2000, Cancer Research 60:6101-6110. The effect of this deregulation is the survival of altered cells which would otherwise have undergone apoptosis in normal conditions. The repetition of these defects associated with unregulated proliferation is thought to be the starting point of cancerous evolution.

These findings as well as numerous others have made possible the emergence of new strategies in drug discovery for targeting cancer. If a small molecule were able to enter the cell and overcome the anti-apoptotic protein over-expression, then it could be possible to reset the apoptotic process. This strategy can have the advantage that it can alleviate the problem of drug resistance which is usually a consequence of apoptotic deregulation (abnormal survival).

Researchers also have demonstrated that platelets also contain the necessary apoptotic machinery (e.g., Bax, Bak, Bcl-xL, Bcl-2, cytochrome c, caspase-9, caspase-3 and APAF-1) to execute programmed cell death through the intrinsic apoptotic pathway. Although circulating platelet production is a normal physiological process, a number of diseases are caused or exacerbated by excess of, or undesired activation of, platelets. The above suggests that therapeutic agents capable of inhibiting anti-apoptotic proteins in platelets and reducing the number of platelets in mammals may be useful in treating pro-thrombotic conditions and diseases that are characterized by an excess of, or undesired activation of, platelets.

Numerous Bcl-xL inhibitors have been developed for treatment of diseases (e.g., cancer) that involve dysregulated apoptotic pathways. However, Bcl-xL inhibitors can act on cells other than the target cells (e.g., cancer cells). For instance, pre-clinical studies have shown that pharmacological inactivation of Bcl-xL reduces platelet half-life and causes thrombocytopenia (see Mason et al., 2007, Cell 128:1173-1186).

Given the importance of Bcl-xL in regulating apoptosis, there remains a need in the art for agents that inhibit Bcl-xL activity, either selectively or non-selectively, as an approach towards the treatment of diseases in which apoptosis is dysregulated via expression or over-expression of anti-apoptotic Bcl-2 family proteins, such as Bcl-xL. Accordingly, new Bcl-xL inhibitors with reduced dose-limiting toxicity are needed.

Additionally, new methods of delivering Bcl-xL inhibitors that limit toxicity are needed. One potential means of delivering a drug to a cell which has not been explored for Bcl-xL inhibitors is delivery through the use of antibody drug conjugates (ADCs). ADCs are formed by chemically linking a cytotoxic drug to a monoclonal antibody through a linker. The monoclonal antibody of an ADC selectively binds to a target antigen of a cell (e.g., cancer cell) and releases the drug into the cell. ADCs have therapeutic potential because they combine the specificity of the antibody and the cytotoxic potential of the drug. Nonetheless, developing ADCs as therapeutic agents has thus far met with limited success owing to a variety of factors such as unfavorable toxicity profiles, low efficacies and poor pharmacological parameters. Accordingly, the development of new ADCs that overcome these problems and can selectively deliver Bcl-xL to target cancer cells would be a significant discovery.

### 3. SUMMARY

It has now been discovered that small molecule inhibitors of Bcl-xL arc efficacious when administered in the form of antibody drug conjugates (ADCs; also called immunoconjugates) that bind to antigens expressed on the surface of cells where inhibition of Bcl-xL and consequent induction of apoptosis would be beneficial. This discovery provides, for the first time, the ability to target Bcl-xL inhibitory therapies to specific cells and/or tissues of interest, potentially lowering serum levels necessary to achieve desired therapeutic benefit and/or avoiding and/or ameliorating potential side effects associated with systemic administration of the small molecule Bcl-xL inhibitors per se.

Accordingly, in one aspect, the present disclosure provides ADCs comprising inhibitors of Bcl-xL useful for, among other things, inhibiting anti-apoptotic Bcl-xL proteins as a therapeutic approach towards the treatment of diseases that involve a dysregulated apoptosis pathway. The ADCs generally comprise small molecule inhibitors of Bcl-xL linked by way of linkers to an antibody that specifically binds an antigen expressed on a target cell of interest.

In another aspect, the present disclosure provides new Bcl-xL inhibitors useful for, among other things, inhibiting anti-apoptotic Bcl-xL proteins as a therapeutic approach towards the treatment of diseases that involve a dysregulated apoptosis pathway. The Bcl-xL inhibitors described herein may be used in the methods described herein, including the various different therapeutic methods, independently from ADCs or as components of ADCs.

The antibody of an ADC may be any antibody that binds, typically but not necessarily specifically, to an antigen expressed on the surface of a target cell of interest. Target cells of interest will generally include cells where induction of apoptosis via inhibition of anti-apoptotic Bcl-xL proteins is desirable, including, by way of example and not limitation, tumor cells that express or over-express Bcl-xL. Target antigens may be any protein, glycoprotein, etc. expressed on the target cell of interest, but will typically be proteins or glycoproteins that are either uniquely expressed on the target cell and not on normal or healthy cells, or that are over-expressed on the target cell as compared to normal or healthy cells, such that the ADCs selectively target specific cells of interest, such as, for example, tumor cells. As is well-known in the art. ADCs bound to certain cell-surface antigens that internalize a bound ADC have certain advantages. Accordingly, in some embodiments, the antigen targeted by the antibody is an antigen that has the ability to internalize an ADC bound thereto into the cell. However, the antigen targeted by the ADC need not be one that internalizes the bound ADC. Bcl-xL inhibitors released outside the target cell or tissue may enter the cell via passive diffusion or other mechanisms to inhibit Bcl-xL.

As will be appreciated by skilled artisans, the specific antigen, and hence antibody, selected will depend upon the identity of the desired target cell of interest. In certain specific therapeutic embodiments, the target antigen for the antibody of the ADC is an antigen that is not expressed on a normal or healthy cell type known or suspected of being dependent, at least in part, on Bcl-xL for survival. In other certain specific therapeutic embodiments, the antibody of the ADC is an antibody suitable for administration to humans.

A vast array of cell-specific antigens useful as therapeutic targets, as well as antibodies that bind these antigens, are known in the art, as are techniques for obtaining additional antibodies suitable for targeting known cell-specific antigens or later-discovered cell-specific antigens. Any of these various different antibodies may be included in the ADCs described herein.

The linkers linking the Bcl-xL inhibitors to the antibody of an ADC may be long, short, flexible, rigid, hydrophobic or hydrophilic in nature, or may comprise segments have different characteristics, such as segments of flexibility, segments of rigidity, etc. The linker may be chemically stable to extracellular environments, for example, chemically stable in the blood stream, or may include linkages that are not stable and release the Bcl-xL inhibitor in the extracellular millieu. In some embodiments, the linker includes linkages that are designed to release the Bcl-xL inhibitor upon internalization of the ADC within the cell. In some specific embodiments, the linker includes linkages designed to cleave and/or immolate or otherwise breakdown specifically or non-specifically inside cells. A wide variety of linkers useful for linking drugs to antibodies in the context of ADCs are known in the art. Any of these linkers, as well as other linkers, may be used to link the Bcl-xL inhibitors to the antibody of the ADCs described herein.

The number of Bcl-xL inhibitors linked to the antibody of an ADC can vary (called the “drug-to-antibody ratio,” or “DAR”), and will be limited only by the number of available attachments sites on the antibody and the number of inhibitors linked to a single linker. Typically, a linker will link a single Bcl-xL inhibitor to the antibody of an ADC. As long as the ADC does not exhibit unacceptable levels of aggregation under the conditions of use and/or storage, ADCs with DARs of twenty, or even higher, are contemplated. In some embodiments, the ADCs described herein may have a DAR in the range of about 1-10, 1-8, 1-6, or 1-4. In certain specific embodiments, the ADCs may have a DAR of 2, 3 or 4. In some embodiments, Bcl-xL inhibitors, linkers and DAR combinations are selected such that the resultant ADC does not aggregate excessively under conditions of use and/or storage.

The new Bcl-xL inhibitors described herein are generally compounds according to the following structural formulae (IIa) and (IIb), below, and/or pharmaceutically acceptable salts thereof, where the various substituents Ar¹, Ar², Z¹, Z^{2a}, Z^{2b}, Z^{2c}, R¹, R², R⁴, R^{11a}, R^{11b}, R¹² and R¹³ are as defined in the Detailed Description section:

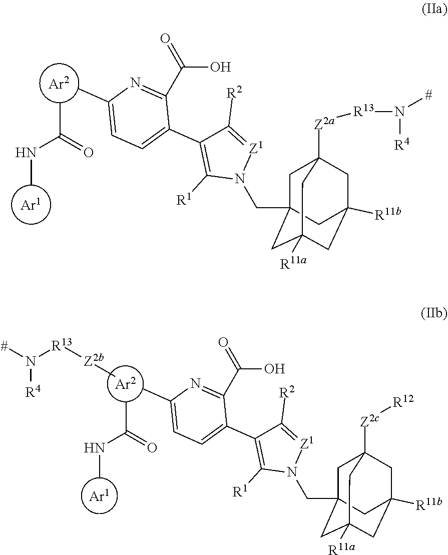

In formulae (IIa) and (IIb) # represents the point of attachment to the linker of an ADC or, for an inhibitor that is not part of an ADC, # represents a hydrogen atom. One embodiment pertains to an antibody drug conjugate (ADC), or pharmaceutically acceptable salt thereof, comprising a drug linked to an antibody by way of a linker, wherein the drug is a Bcl-xL inhibitor according to formulae (IIa) or (IIb) in which the # represents the point of attachment to the linker.

In some embodiments, the ADCs described herein are generally compounds according to structural formula (I):

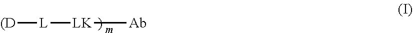

where Ab represents the antibody, D represents the drug (here, a Bcl-xL inhibitor). L represents the linker linking the drug D to the antibody Ab, LK represents a linkage formed between a functional group on linker L and a complementary functional group on antibody Ab, and m represents the number of linker-drug units linked to the antibody.

In certain specific embodiments, the ADCs are compounds according to structural formulae (Ia) or (Ib) below, where the various substituents Ar¹, Ar², Z¹, Z^{2a}, Z^{2b}, Z^{2c}, R¹, R², R⁴, R^{11a}, R^{11b}, R¹² and R¹³ are as previously defined for formulae (IIa) and (IIb), respectively, Ab and L are as defined for structural formulae (I), LK represents a linkage formed between a functional group on linker L and a complementary functional group on antibody Ab, and m is an integer ranging from 1 to 20, and in some embodiments from 2 to 8, and in some embodiments 1 to 8, and in some embodiments 2, 3, or 4:

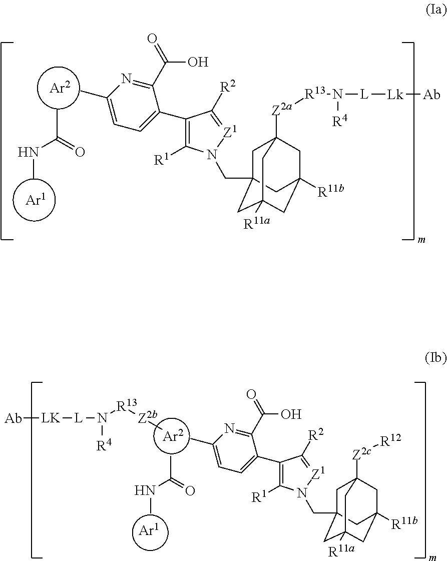

In another aspect, the present disclosure provides intermediate synthons useful for synthesizing the ADCs described herein, as well as methods for synthesizing the ADCs. The intermediate synthons generally comprise Bcl-xL inhibitors linked to a linker moiety that includes a functional group capable of linking the synthon to an antibody. The synthons are generally compounds according to structural formula (III), below, or salts thereof, where D is a Bcl-xL inhibitor as previously described herein, L is a linker as previously described and R^{x} comprises a functional group capable of conjugating the synthon to a complementary functional group on an antibody:

D-L-R^{x} (III)

In certain specific embodiments, the intermediate synthons are compounds according to structural formulae (IIIa) and (IIIb), below, or salts thereof, where the various substituents Ar¹, Ar², Z¹, Z^{2a}, Z^{2b}, Z^{2c}, R¹, R², R⁴, R^{11a}, R^{11b}, R¹² and R¹³ are as previously defined for structural formulae (IIa) and (IIb), L is a linker as previously described and R^{x} is a functional group as described above:

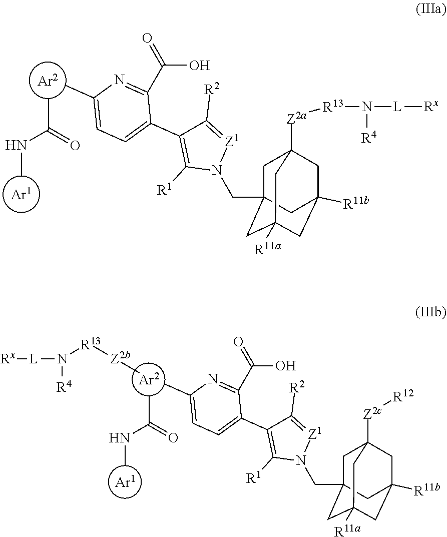

To synthesize an ADC, intermediate synthons according to structural formulae (III) or (IIIa) or (IIIb), or salts thereof, are contacted with an antibody of interest under conditions in which functional group R^{x} reacts with a complementary functional group on the antibody to form a covalent linkage. The identity of group R^{x} will depend upon the desired coupling chemistry and the complementary groups on the antibody to which the synthons will be attached. Numerous groups suitable for conjugating molecules to antibodies are known in the art. Any of these groups may be suitable for R^{x}. Non-limiting exemplary functional groups (R^{x}) include NHS-esters, maleimides, haloacetyls, isothiocyanates, vinyl sulfones and vinyl sulfonamides.

In another aspect, the present disclosure provides compositions including the Bcl-xL inhibitors or ADCs described herein. The compositions generally comprise one or more Bcl-xL inhibitors or ADCs as described herein, and/or salts thereof, and one or more excipients, carriers or diluents. The compositions may be formulated for pharmaceutical use, or other uses. In a specific embodiment, the composition is formulated for pharmaceutical use and comprises a Bcl-xL inhibitor according to structural formula (IIa) or (IIb), or a pharmaceutically acceptable salt thereof, where # is hydrogen. In another embodiment, the composition is formulated for pharmaceutical use and comprises an ADC according to structural formula (IIIa) or (IIIb), or a pharmaceutically acceptable salt thereof, and one or more pharmaceutically acceptable excipients, carriers or diluents.

Bcl-xL inhibitory compositions formulated for pharmaceutical use may be packaged in bulk form suitable for multiple administrations, or may be packaged in the term of unit doses, such as for example tablets or capsules, suitable for a single administration. Likewise, ADC compositions formulated for pharmaceutical use may be packaged in bulk form suitable for multiple administrations, or may be packaged in the form of unit doses suitable for a single administration. Whether packaged in bulk or in the form of unit doses, the ADC composition may be a dry composition, such as a lyophilate, or a liquid composition. Unit dosage liquid ADC compositions may be conveniently packaged in the form of syringes pre-filled with an amount of ADC suitable for a single administration.

In still another aspect, the present disclosure provides methods of inhibiting anti-apoptotic Bcl-xL proteins. The method generally involves contacting an ADC as described herein, for example, an ADC according to structural formula (Ia) or (Ib), or a salt thereof, with a target cell that expresses or overexpresses Bcl-xL and an antigen for the antibody of the ADC under conditions in which the antibody binds the antigen on the target cell. Depending upon the antigen, the ADC may become internalized into the target cell. The method may be carried out in vitro in a cellular assay to inhibit Bcl-xL activity, or in vivo as a therapeutic approach towards the treatment of diseases in which inhibition of Bcl-xL activity is desirable. The method may alternatively involve contacting a cell that expresses or over-expresses Bcl-xL with a Bcl-xL inhibitor, such as an inhibitor according to structural formula (IIa) or (IIb), where # is hydrogen, or a salt thereof.

In still another aspect, the present disclosure provides methods of inducing apoptosis in cells. The method generally involves contacting an ADC as described herein, for example, an ADC according to structural formula (Ia) or (Ib), or a salt thereof, with a target cell that expresses or overexpresses Bcl-xL and an antigen for the antibody of the ADC under conditions in which the antibody binds the antigen on the target cell. Depending upon the antigen, the ADC may become internalized into the target cell. The method may be carried out in vitro in a cellular assay to induce apoptosis, or in vivo as a therapeutic approach towards the treatment of diseases in which induction of apoptosis in specific cells would be beneficial. The method may alternatively involve contacting a cell that expresses or over-expresses Bcl-xL with a Bcl-xL inhibitor, for example an inhibitor according to structural formula (IIa) or (IIb), where # is hydrogen, or a salt thereof. In one embodiment, the antibody of the ADC described herein binds a cell surface receptor or a tumor associated antigen expressed on a tumor cell. In another embodiment, the antibody of the ADC described herein binds one of the cell surface receptors or tumor associated antigens selected from EGFR, EpCAM and NCAM1. In another embodiment, the antibody of the ADC described herein binds EGFR, EpCAM or NCAM1. In another embodiment, the antibody of the ADC described herein binds EpCAM or NCAM1. In another embodiment, the antibody of the ADC described herein binds EpCAM. In another embodiment, the antibody of the ADC described herein binds EGFR. In another embodiment, the antibody of the ADC described herein binds NCAM-1.

In yet another aspect, the present disclosure provides methods of treating disease in which inhibition of Bcl-xL and/or induction of apoptosis would be desirable. As will be discussed more thoroughly in the Detailed Description section, a wide variety of diseases are mediated, at least in part, by dysregulated apoptosis stemming, at least in part, by expression or over-expression of anti-apoptotic Bcl-xL proteins. Any of these diseases may be treated or ameliorated with the Bcl-xL inhibitors or ADCs described herein.

The methods include administering to a subject suffering from a disease mediated, at least in part by expression or over-expression of Bcl-xL, an amount of a Bcl-xL inhibitor or ADC described herein effective to provide therapeutic benefit. For ADCs, the identity of the antibody of the ADC administered will depend upon the disease being treated. The therapeutic benefit achieved with the Bcl-xL inhibitors and ADCs described herein will also depend upon the disease being treated. In certain instances, the Bcl-xL inhibitory or ADC may treat or ameliorate the specific disease when administered as monotherapy. In other instances, the Bcl-xL inhibitor or ADC may be part of an overall treatment regimen including other agents that, together with the Bcl-xL inhibitor or ADC treat or ameliorate the disease.

For example, elevated expression levels of Bcl-xL have been associated with resistance to chemotherapy and radiation therapy in cancers (Datta et al., 1995, Cell Growth Differ 6:363-370; Amundson et al., 2000, Cancer Res 60:6101-6110; Haura et al., 2004, Clin Lung Cancer 6:113-122). In the context of treating cancers, data disclosed herein establish that ADCs may be effective as monotherapy or may be effective when administered adjunctive to, or with, other targeted or non-targeted chemotherapeutic agents and/or radiation therapy. While not intending to be bound by any theory of operation, it is believed that inhibition of Bcl-xL activity with the Bcl-xL inhibitors and ADCs described herein in tumors that have become resistant to targeted or non-targeted chemo- and/or radiation therapies will “sensitize” the tumors such that they are again susceptible to the chemotherapeutic agents and/or radiation treatment.

Accordingly, in the context of treating cancers, “therapeutic benefit” includes administration of the Bcl-xL inhibitors and ADCs described herein adjunctive to, or with, targeted or non-targeted chemotherapeutic agents and/or radiation therapy, either in patients that have not yet begun the chemo- and/or radiation therapeutic regimens, or in patients that have exhibited resistance (or are suspected or becoming resistant) to the chemo- and/or radiation therapeutic regimens, as a means of sensitizing the tumors to the chemo- and/or radiation therapy. One embodiment pertains to a method of sensitizing a tumor to standard cytotoxic agents and/or radiation, comprising contacting the tumor with an ADC described herein that is capable of binding the tumor, in an amount effective to sensitize the tumor cell to a standard cytotoxic agent and/or radiation. Another embodiment pertains to a method of sensitizing a tumor to standard cytotoxic agents and/or radiation, comprising contacting the tumor with an ADC described herein that is capable of binding the tumor, in an amount effective to sensitize the tumor cell to a standard cytotoxic agent and/or radiation in which the tumor has become resistant to treatment with standard cytotoxic agents and/or radiation. Another embodiment pertains to a method of sensitizing a tumor to standard cytotoxic agents and/or radiation, comprising contacting the tumor with an ADC described herein that is capable of binding the tumor, in an amount effective to sensitize the tumor cell to a standard cytotoxic agent and/or radiation in which the tumor has not been previously exposed to standard cytotoxic agents and/or radiation therapy.

### 4. DETAILED DESCRIPTION

The present disclosure concerns new Bcl-xL inhibitors, ADCs comprising the inhibitors, synthons useful for synthesizing the ADCs, compositions comprising the inhibitors or ADCs, and various methods of using the inhibitors and ADCs.

As will be appreciated by skilled artisans, the ADCs disclosed herein are “modular” in nature. Throughout the instant disclosure, various specific embodiments of the various “modules” comprising the ADCs, as well as the synthons useful for synthesizing the ADCs, are described. As specific non-limiting examples, specific embodiments of antibodies, linkers, and Bcl-xL inhibitors that may comprise the ADCs and synthons are described. It is intended that all of the specific embodiments described may be combined with each other as though each specific combination were explicitly described individually.

It will also be appreciated by skilled artisans that the various Bcl-xL inhibitors. ADCs and/or ADC synthons described herein may be in the form of salts, and in certain embodiments, particularly pharmaceutically acceptable salts. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as a bromide, chloride, or fluoride.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, etc. Base addition salts include those derived from inorganic bases, such as ammonium and alkali or alkaline earth metal hydroxides, carbonates, bicarbonates, and the like.

In the disclosure below, if both structural diagrams and nomenclature are included and if the nomenclature conflicts with the structural diagram, the structural diagram controls.

### 4.1. Definitions

Unless otherwise defined herein, scientific and technical terms used in connection with the present disclosure shall have the meanings that are commonly understood by those of ordinary skill in the art.

Various chemical substituents are defined below. In some instances, the number of carbon atoms in a substituent (e.g., alkyl, alkanyl, alkenyl, alkynyl, cycloalkyl, heterocyclyl, heteroaryl, and aryl) is indicated by the prefix “Cₓ-C_{y},” wherein x is the minimum and y is the maximum number of carbon atoms. Thus, for example, “C₁-C₆ alkyl” refers to an alkyl containing from 1 to 6 carbon atoms. Illustrating further, “C₃-C₈ cycloalkyl” means a saturated hydrocarbyl ring containing from 3 to 8 carbon ring atoms. If a substituent is described as being “substituted,” a hydrogen atom on a carbon or nitrogen is replaced with a non-hydrogen group. For example, a substituted alkyl substituent is an alkyl substituent in which at least one hydrogen atom on the alkyl is replaced with a non-hydrogen group. To illustrate, monofluoroalkyl is alkyl substituted with a fluoro radical, and difluoroalkyl is alkyl substituted with two fluoro radicals. It should be recognized that if there is more than one substitution on a substituent, each substitution may be identical or different (unless otherwise stated). If a substituent is described as being “optionally substituted”, the substituent may be either (1) not substituted or (2) substituted. Possible substituents include, but are not limited to, C₁-C₆ alkyl, C₂-C₆ alkenyl, C₂-C₆ alkynyl, aryl, cycloalkyl, heterocyclyl, heteroaryl, halogen, C₁-C₆ haloalkyl, oxo, —CN, NO₂, —OR^{xa}, —OC(O)R^{z}, —OC(O)N(R^{xa})₂, —SR^{xa}, —S(O)₂R^{xa}, —S(O)₂N(R^{xa})₂, —C(O)R^{xa}, —C(O)OR^{xa}, —C(O)N(R^{xa})₂, —C(O)N(R^{xa})S(O)₂R^{z}, —N(R^{xa})₂, —N(R^{xa})C(O)R^{z}, —N(R^{xa})S(O)₂R^{z}, —N(R^{xa})C(O)O(R^{z}), —N(R^{xa})C(O)N(R^{xa})₂, —N(R^{xa})S(O)₂N(R^{xa})₂, —(C₁-C₆ alkylenyl)-CN, —(C₁-C₆ alkylenyl)-OR^{xa}, —(C₁-C₆ alkylenyl)-OC(O)R^{z}, —(C₁-C₆ alkylenyl)-OC(O)N(R^{xa})₂, —(C₁-C₆ alkylenyl)-SR^{xa}, —(C₁-C₆ alkylenyl)-S(O)₂R^{xa}, —(C₁-C₆ alkylenyl)-S(O)₂N(R^{xa})₂, —(C₁-C₆ alkylenyl)-C(O)R^{xa}, —(C₁-C₆ alkylenyl)-C(O)OR^{xa}, —(C₁-C₆ alkylenyl)-C(O)N(R^{xa})₂, —(C₁-C₆ alkylenyl)-C(O)N(R^{xa})S(O)₂R^{z}, —(C₁-C₆ alkylenyl)-N(R^{xa})₂, —(C₁-C₆ alkylenyl)-N(R^{xa})C(O)R^{z}, —(C₁-C₆ alkylenyl)-N(R^{xa})S(O)₂R^{z}, —(C₁-C₆ alkylenyl)-N(R^{xa})C(O)O(R^{z}), —(C₁-C₆ alkylenyl)-N(R^{xa})C(O)N(R^{xa})₂, or —(C₁-C₆ alkylenyl)-N(R^{xa})S(O)₂N(R^{xa})₂; wherein R^{xa}, at each occurrence, is independently hydrogen, aryl, cycloalkyl, heterocyclyl, heteroaryl, C₁-C₆ alkyl, or C₁-C₆ haloalkyl; and R^{z}, at each occurrence, is independently aryl, cycloalkyl, heterocyclyl, heteroaryl, C₁-C₆ alkyl or C₁-C₆ haloalkyl.

Various Bcl-xL inhibitors, ADCs, and synthons are described in some embodiments herein by reference to structural formulae including substituent groups. It is to be understood that the various groups comprising the substituents may be combined as valence and stability permit. Combinations of substituents and variables envisioned by this disclosure are only those that result in the formation of stable compounds. As used herein, the term “stable” refers to compounds that possess stability sufficient to allow manufacture and that maintain the integrity of the compound for a sufficient period of time to be useful for the purpose detailed herein.

As used herein, the following terms are intended to have the following meanings:

The term “alkoxy” refers to a group of the formula —OR^{a}, where R^{a′} is an alkyl group. Representative alkoxy groups include methoxy, ethoxy, propoxy, tert-butoxy and the like.

The term “alkoxyalkyl” refers to an alkyl group substituted with an alkoxy group and may be represented by the general formula —R^{b}OR^{a} where R^{b} is an alkylene group and R^{a} is an alkyl group.

The term “alkyl” by itself or as part of another substituent refers to a saturated or unsaturated branched, straight-chain or cyclic monovalent hydrocarbon radical that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl.” “alkenyl” and/or “alkynyl” is used, as defined below. The term “lower alkyl” refers to alkyl groups with 1 to 6 carbons.

The term “alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

The term “alkenyl” by itself or as part of another substituent refers to an unsaturated branched, straight-chain or cyclic alkyl having at least one carbon-carbon double bond derived by the removal of one hydrogen atom from a single carbon atom of a parent alkene. Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

The term “alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

The term “alkylamine” refers to a group of the formula —NHR^{a} and “dialkylamine” refers to a group of the formula —NR^{a}R^{a}, where each R^{a} is, independently of the others, an alkyl group.

The term “alkylene” refers to an alkane, alkene or alkyne group having two terminal monovalent radical centers derived by the removal of one hydrogen atom from each of the two terminal carbon atoms. Typical alkylene groups include, but are not limited to, methylene; and saturated or unsaturated ethylene; propylene; butylene; and the like. The term “lower alkylene” refers to alkylene groups with 1 to 6 carbons.

The term “heteroalkylene” refers to a divalent alkylene having one or more —CH— groups replaced with a thio, oxy, or —NR³— where R³ is selected from hydrogen, lower alkyl and lower heteroalkyl. The heteroalkylene can be linear, branched, cyclic, bicyclic, or a combination thereof and can include up to 10 carbon atoms and up to 4 heteroatoms. The term “lower heteroalkylene” refers to alkylene groups with 1 to 4 carbon atoms and 1 to 3 heteroatoms.

The term “aryl” means an aromatic carbocyclyl containing from 6 to 14 carbon ring atoms. An aryl may be monocyclic or polycyclic (i.e., may contain more than one ring). In the case of polycyclic aromatic rings, only one ring the polycyclic system is required to be aromatic while the remaining ring(s) may be saturated, partially saturated or unsaturated. Examples of aryls include phenyl, naphthalenyl, indenyl, indanyl, and tetrahydronaphthyl.

The term “arylene” refers to an aryl group having two monovalent radical centers derived by the removal of one hydrogen atom from each of the two ring carbons. An exemplary arylene group is a phenylene.

An alkyl group may be substituted by a “carbonyl” which means that two hydrogen atoms from a single alkanylene carbon atom are removed and replaced with a double bond to an oxygen atom.

The prefix “halo” indicates that the substituent which includes the prefix is substituted with one or more independently selected halogen radicals. For example, haloalkyl means an alkyl substituent in which at least one hydrogen radical is replaced with a halogen radical. Typical halogen radicals include chloro, fluoro, bromo and iodo. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The term “haloalkoxy” refers to a group of the formula —OR^{c}, where R^{c} is a haloalkyl.

The terms “heteroalkyl.” “heteroalkanyl,” “heteroalkenyl,” “heteroalkynyl,” and “heteroalkylene” refer to alkyl, alkanyl, alkenyl, alkynyl, and alkylene groups, respectively, in which one or more of the carbon atoms, e.g., 1, 2 or 3 carbon atoms, are each independently replaced with the same or different heterotoms or heteroatomic groups. Typical heteroatoms and/or heteroatomic groups which can replace the carbon atoms include, but are not limited to, —O—, —S—, —S—O—, —NR^{c}—, —PH, —S(O)—, —S(O)₂—, —S(O)NR^{c}—, —S(O)₂NR^{c}—, and the like, including combinations thereof, where each R^{c} is independently hydrogen or C₁-C₆ alkyl. The term “lower heteroalkyl” refers to between 1 and 4 carbon atoms and between 1 and 3 heteroatoms.

The terms “cycloalkyl” and “heterocyclyl” refer to cyclic versions of “alkyl” and “heteroalkyl” groups, respectively. For heterocyclyl groups, a heteroatom can occupy the position that is attached to the remainder of the molecule. A cycloalkyl or heterocyclyl ring may be a single-ring (monocyclic) or have two or more rings (bicyclic or polycyclic).

Monocyclic cycloalkyl and heterocyclyl groups will typically contains from 3 to 7 ring atoms, more typically from 3 to 6 ring atoms, and even more typically 5 to 6 ring atoms. Examples of cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Examples of monocyclic heterocyclyls include, but are not limited to, oxetane, furanyl, dihydrofuranyl, tetrahydrofuranyl, tetrahydropyranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl (furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), 1,4-dioxanyl, dioxothiomorpholinyl, oxathiazolyl, oxathiolyl, oxathiolanyl, pyranyl, dihydropyranyl, thiopyranyl, tetrahydrothiopyranyl, pyridinyl (azinyl), piperidinyl, diazinyl (including pyridazinyl (1,2-diazinyl), pyrimidinyl (1,3-diazinyl), or pyrazinyl (1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, diazepinyl, pyridonyl (including pyrid-2(1H)-onyl and pyrid-4(1H)-onyl), furan-2(5H)-onyl, pyrimidonyl (including pyramid-2(1H)-onyl and pyramid-4(3H)-onyl), oxazol-2(3H)-onyl, 1H-imidazol-2(3H)-onyl, pyridazin-3(2H)-onyl, and pyrazin-2(1H)-onyl.

Polycyclic cycloalkyl and heterocyclyl groups contain more than one ring, and bicyclic cycloalkyl and heterocyclyl groups contain two rings. The rings may be in a bridged, fused or spiro orientation. Polycyclic cycloalkyl and heterocyclyl groups may include combinations of bridged, fused and/or spiro rings. In a spirocyclic cycloalkyl or heterocyclyl, one atom is common to two different rings. An example of a spirocycloalkyl is spiro[4.5]decane and an example of a spiroheterocyclyls is a spiropyrazoline.

In a bridged cycloalkyl or heterocyclyl, the rings share at least two common non-adjacent atoms. Examples of bridged cycloalkyls include, but are not limited to, adamantyl and norbomanyl rings. Examples of bridged heterocyclyls include, but are not limited to, 2-oxatricyclo[3.3.1.1^{3,7}]decanyl.

In a fused-ring cycloalkyl or heterocyclyl, two or more rings are fused together, such that two rings share one common bond. Examples of fused-ring cycloalkyls include decalin, naphthylene, tetralin, and anthracene. Examples of fused-ring heterocyclyls containing two or three rings include imidazopyrazinyl (including imidazo[1,2-a]pyrazinyl), imidazopyridinyl (including imidazo[1,2-a]pyridinyl), imidazopyridazinyl (including imidazol[1,2-b]pyridazinyl), thiazolopyridinyl (including thiazolo[5,4-c]pyridinyl, thiazolo[5,4-b]pyridinyl, thiazolo[4,5-b]pyridinyl, and thiazolo[4,5-c]pyridinyl), indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as dihydrochromenyl, tetrahydroisoquinolinyl, indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), benzo[d]thiazolyl, and benzisoxazinyl (including 1,2-benzisoxazinyl or 1,4-benzisoxazinyl).

The term “heteroaryl” refers to an aromatic heterocyclyl containing from 5 to 14 ring atoms. A heteroaryl may be a single ring or 2 or 3 fused rings. Examples of heteroaryls include 6-membered rings such as pyridyl, pyrazyl, pyrimidinyl, pyridazinyl, and 1,3,5-, 1,2,4- or 1,2,3-triazinyl; 5-membered ring substituents such as triazolyl, pyrrolyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as imidazopyrazinyl (including imidazo[1,2-a]pyrazinyl)imidazopyridinyl (including imidazo[1,2-a]pyridinyl), imidazopyridazinyl (including imidazo[1,2-b]pyridazinyl), thiazolopyridinyl (including thiazolo[5,4-c]pyridinyl, thiazolo[5,4-b]pyridinyl, thiazolo[4,5-b]pyridinyl, and thiazolo[4,5-c]pyridinyl), benzo[d]thiazolyl, benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl. Heteroaryls may also be heterocycles having aromatic (4N+2 pi electron) resonance contributors such as pyridonyl (including pyrid-2(1H)-onyl and pyrid-4(1H)-onyl), pyrimidonyl (including pyramid-2(1H)-onyl and pyramid-4(3H)-onyl), pyridazin-3(2H)-onyl and pyrazin-2(1H)-onyl.

The term “sulfonate” as used herein means a salt or ester of a sulfonic acid.

The term “methyl sulfonate” as used herein means a methyl ester of a sulfonic acid group.

The term “carboxylate” as used herein means a salt or ester of a caboxylic acid.

The term “polyol”, as used herein, means a group containing more than two hydroxyl groups independently or as a portion of a monomer unit. Polyols include, but are not limited to, reduced C₂-C₆ carbohydrates, ethylene glycol, and glycerin.

The term “sugar” when used in context of “G¹” includes O-glycoside, N-glycoside, S-glycoside and C-glycoside (C-glycoslyl) carbohydrate derivatives of the monosaccharide and disaccharide classes and may originate from naturally-occurring sources or may be synthetic in origin. For example “sugar” when used in context of “G¹” includes derivatives such as but not limited to those derived from glucuronic acid, galacturonic acid, galactose, and glucose among others. Suitable sugar substitutions include but are not limited to hydroxyl, amine, carboxylic acid, sulfonic acid, phosphonic acid, esters, and ethers.

The term “NHS ester” means the N-hydroxysuccinimide ester derivative of a carboxylic acid.

The term “amine” includes primary, secondary and tertiary aliphatic amines, including cyclic versions.

The term salt when used in context of “or salt thereof” include salts commonly used to form alkali metal salts and to form addition salts of free acids or free bases. In general, these salts typically may be prepared by conventional means by reacting, for example, the appropriate acid or base with a compound of the invention

Where a salt is intended to be administered to a patient (as opposed to, for example, being in use in an m vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. The term “pharmaceutically acceptable salt” includes salts commonly used to form alkali metal salts and to form addition salts of free acids or free bases. In general, these salts typically may be prepared by conventional means by reacting, for example, the appropriate acid or base with a compound of the invention.

### 4.2. Exemplary Embodiments

As noted in the Summary, aspects of the disclosure concern Bcl-xL inhibitors and ADCs comprising Bcl-xL inhibitors linked to antibodies by way of linkers. In specific embodiments, the ADCs are compounds according to structural formula (I), below, or salts thereof, wherein Ab represents the antibody. D represents a Bcl-xL inhibitor (drug), L represents a linker, LK represents a linkage formed between a reactive functional group on linker L and a complementary functional group on antibody Ab and m represents the number of D-L-LK units linked to the antibody:

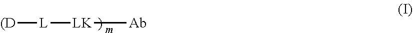

Specific embodiments of various Bcl-xL inhibitors per se, and various Bcl-xL inhibitors (D), linkers (L) and antibodies (Ab) that can comprise the ADCs described herein, as well as the number of Bcl-xL inhibitors linked to the ADCs, are described in more detail below.

### 4.3. Bcl-xL Inhibitors

One aspect of the instant disclosure concerns new Bcl-xL inhibitors. The Bcl-xL inhibitors may be used as compounds or salts per se in the various methods described herein, or may be included as a component part of an ADC.

Specific embodiments of Bcl-xL inhibitors that may be used in unconjugated form, or that may be included as part of an ADC include compounds according to structural formula (IIa) or (IIb):

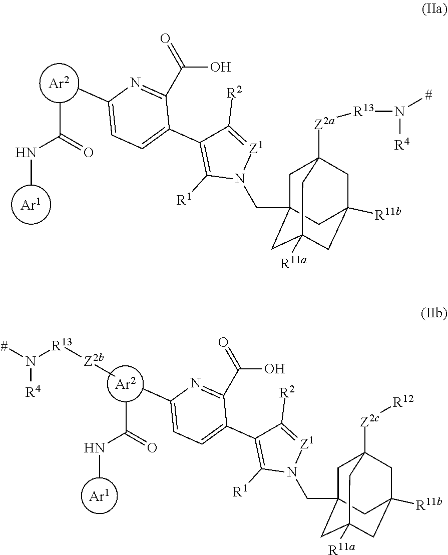

or salts thereof, wherein:
Ar¹ is selected from

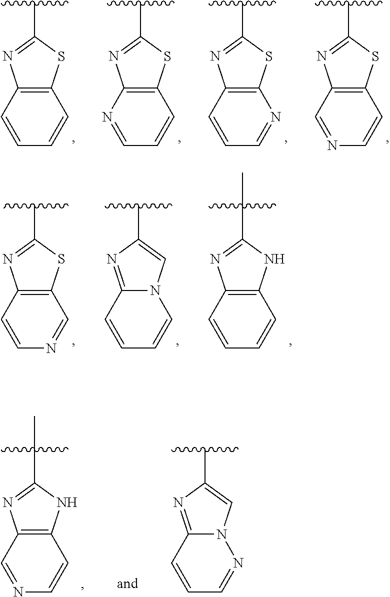

and is optionally substituted with one or more substituents independently selected from halo, hydroxy, nitro, lower alkyl, lower heteroalkyl, alkoxy, amino, cyano and halomethyl;
Ar² is selected from

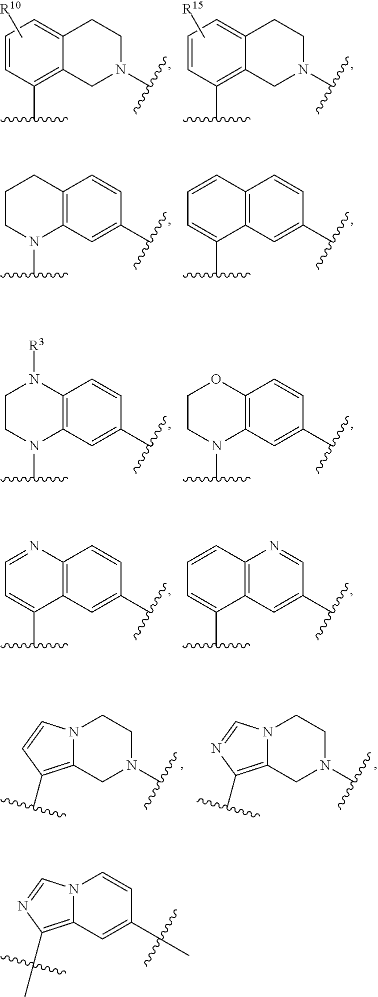

and is optionally substituted with one or more substituents independently selected from halo, hydroxy, nitro, lower alkyl, lower heteroalkyl, alkoxy, amino, cyano and halomethyl, wherein the #-N(R⁴)—R¹³—Z^{2b}— substituent of formula (IIb) is attached to Ar² at any Ar² atom capable of being substituted;
Z¹ is selected from N, CH, C-halo and C—CN;
Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O;
R¹ is selected from hydrogen, methyl, halo, halomethyl, ethyl and cyano;
R² is selected from hydrogen, methyl, halo, halomethyl and cyano;
R³ is selected from hydrogen, lower alkyl and lower heteroalkyl;
R⁴ is selected from hydrogen, lower alkyl, monocyclic cycloalkyl, monocyclic heterocyclyl, lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, lower heteroalkyl are optionally substituted with one or more halo, cyano, alkoxy, monocyclic cycloalkyl, monocyclic heterocyclyl, NC(O)CR^{6a}R^{6b}, NS(O)CR^{6a}R^{6b}, NS(O₂)CR^{6a}R^{6b}, S(O₂)CR^{6a}R^{6b} or S(O₂)NH₂ groups;
R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;
R¹⁰ is selected from cyano, OR¹⁴, SR¹⁴, SOR¹⁴, SO₂R¹⁴, SO₂NR^{14a}R^{14b}, NR¹⁴R^{14b}, NC(O)R¹⁴ and NSO₂R¹⁴;
R^{11a} and R^{11b} are each, independently of one another, selected from hydrogen, halo, methyl, ethyl, halomethyl, hydroxyl, methoxy, CN, and SCH₃;
R¹² is selected from hydrogen, halo, cyano, lower alkyl, lower heteroalkyl, cycloalkyl, or heterocyclyl, wherein the alkyl, heteroalkyl, cycloalkyl, or heterocyclyl are optionally substituted with one or more halo, cyano, alkoxy, monocyclic cycloalkyl, monocyclic heterocyclyl, NC(O)CR^{6a}R^{6b}, NS(O)CR^{6a}R^{6b}, NS(O₂)CR^{6a}R^{6b} or S(O₂)CR^{6a}R^{6b} groups;

R¹³ is selected from a bond, optionally substituted lower alkylene, optionally substituted lower heteroalkylene, optionally substituted cycloalkyl or optionally substituted heterocyclyl;
R¹⁴ is selected from hydrogen, optionally substituted lower alkyl and optionally substituted lower heteroalkyl;
R^{14a} and R^{14b} are each, independently of one another, selected from hydrogen, optionally substituted lower alkyl, optionally substituted lower heteroalkyl, or are taken together with the nitrogen atom to which they are bonded to form a monocyclic cycloalkyl or monocyclic heterocyclyl ring;
R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl C₂₋₄ alkynyl, C₁₋₄ haloalkyl or C₁₋₄ hydroxyalkyl, wherein the R⁴ C₁₋₉ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and
# represents a point of attachment to a linker or a hydrogen atom.

Specific embodiments of Bcl-xL inhibitors that may be used in unconjugated form, or that may be included as part of an ADC include compounds according to structural formula (IIa) or (IIb):

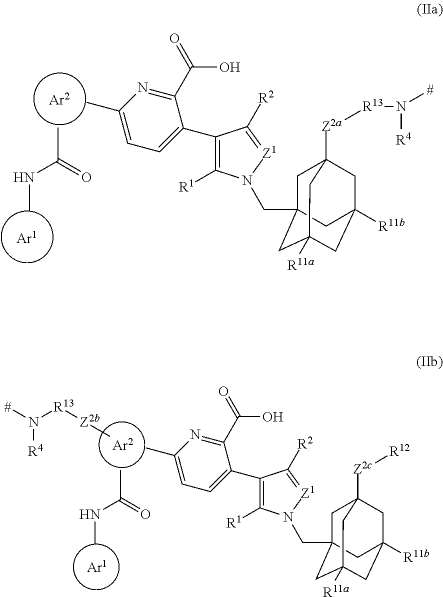

or salts thereof, wherein:
Ar¹ is selected from

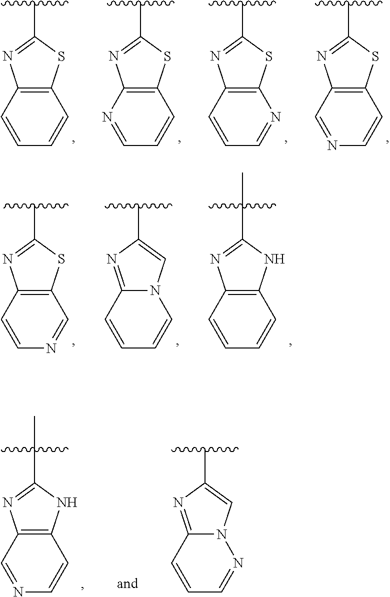

and is optionally substituted with one or more substituents independently selected from halo, hydroxy, nitro, lower alkyl, lower heteroalkyl, alkoxy, amino, cyano and halomethyl;
Ar² is selected from

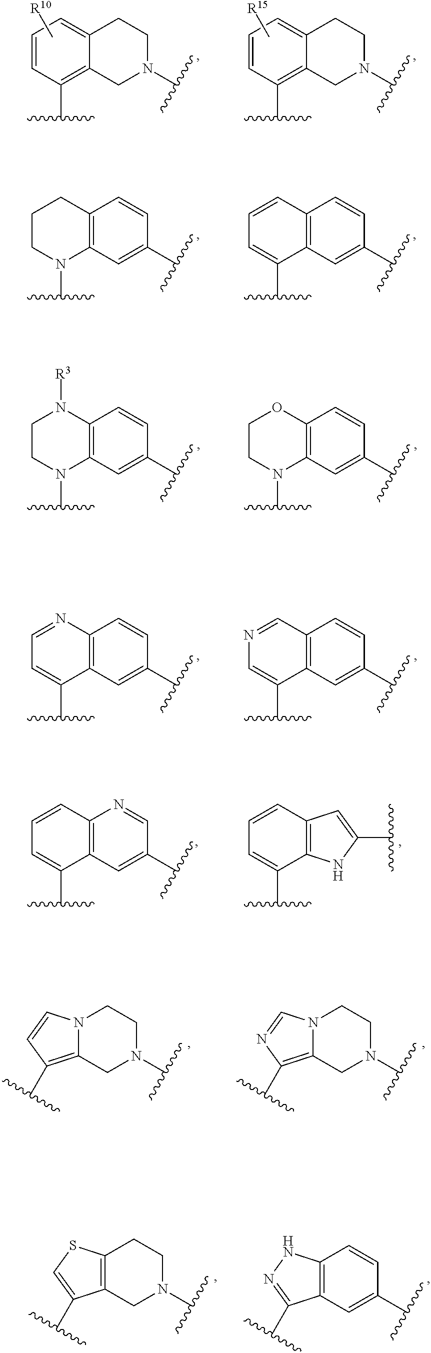

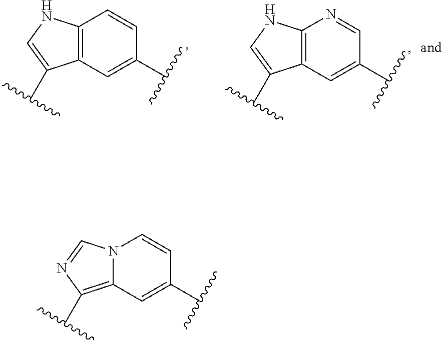

and is optionally substituted with one or more substituents independently selected from halo, hydroxy, nitro, lower alkyl, lower heteroalkyl, alkoxy, amino, cyano and halomethyl, wherein the #-N(R⁴)—R¹³—Z^{2b}— substituent of formula (IIb) is attached to Ar² at any Ar² atom capable of being substituted;
Z¹ is selected from N, CH, C-halo and C—CN;
Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O;
R¹ is selected from hydrogen, methyl, halo, halomethyl, ethyl and cyano;
R² is selected from hydrogen, methyl, halo, halomethyl and cyano;
R³ is selected from hydrogen, lower alkyl and lower heteroalkyl;
R⁴ is selected from hydrogen, lower alkyl, monocyclic cycloalkyl, monocyclic heterocyclyl, and lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, and lower heteroalkyl are optionally substituted with one or more halo, cyano, hydroxy, alkoxy, monocyclic cycloalkyl, monocyclic heterocyclyl, C(O)NR^{6a}R^{6b}, S(O₂)NR^{6a}R^{6b}, NHC(O)CHR^{6a}R^{6b}, NHS(O)CHR^{6a}R^{6b}, NHS(O₂)CHR^{6a}R^{6b}, S(O₂)CHR^{6a}R^{6b} or S(O₂)NH₂ groups;
R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;
R¹⁰ is selected from cyano, OR¹⁴, SR¹⁴, SOR¹⁴, SO₂R¹⁴, SO₂NR^{14a}R^{14b}, NR^{14a}R^{14b}, NHC(O)R¹⁴ and NHSO₂R¹⁴;
R^{11a} and R^{11b} are each, independently of one another, selected from hydrogen, halo, methyl, ethyl, halomethyl, hydroxyl, methoxy, CN, and SCH₃;
R¹² is selected from hydrogen, halo, cyano, lower alkyl, lower heteroalkyl, cycloalkyl, and heterocyclyl, wherein the alkyl, heteroalkyl, cycloalkyl, and heterocyclyl are optionally substituted with one or more halo, cyano, alkoxy, monocyclic cycloalkyl, monocyclic heterocyclyl, NHC(O)CHR^{6a}R^{6b}, NHS(O)CHR^{6a}R^{6b}, NHS(O₂)CHR^{6a}R^{6b} or S(O₂)CHR^{6a}R^{6b} groups;
R¹³ is selected from a bond, optionally substituted lower alkylene, optionally substituted lower heteroalkylene, optionally substituted cycloalkyl or optionally substituted heterocyclyl;
R¹⁴ is selected from hydrogen, optionally substituted lower alkyl and optionally substituted lower heteroalkyl;
R^{14a} and R^{14b} are each, independently of one another, selected from hydrogen, optionally substituted lower alkyl, and optionally substituted lower heteroalkyl, or arc taken together with the nitrogen atom to which they are bonded to form an optionally substituted monocyclic cycloalkyl or monocyclic heterocyclyl ring;
R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and # represents a point of attachment to a linker or a hydrogen atom.

Another embodiment of Bcl-xL inhibitors that may be used in unconjugated form, or that may be included as part of an ADC include compounds according to structural formula (IIa) or (IIb):

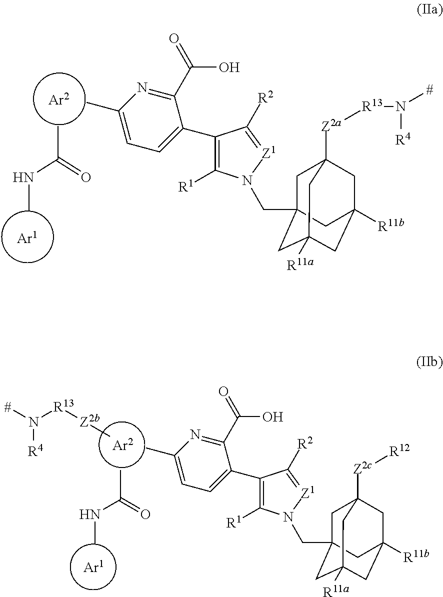

or salts thereof, wherein:
Ar¹ is selected from

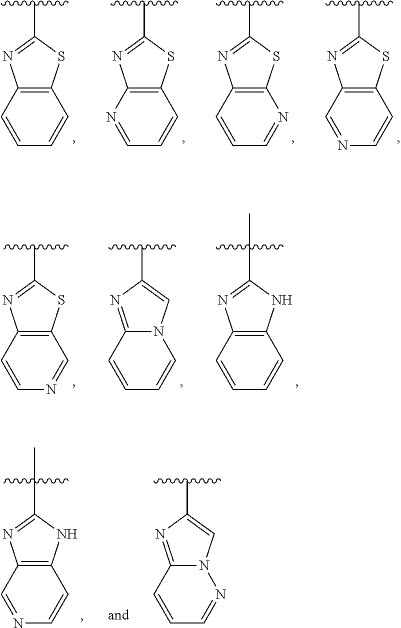

and is optionally substituted with one or more substituents independently selected from halo, hydroxy, nitro, lower alkyl, lower heteroalkyl, alkoxy, amino, cyano and halomethyl;
Ar¹ is selected from

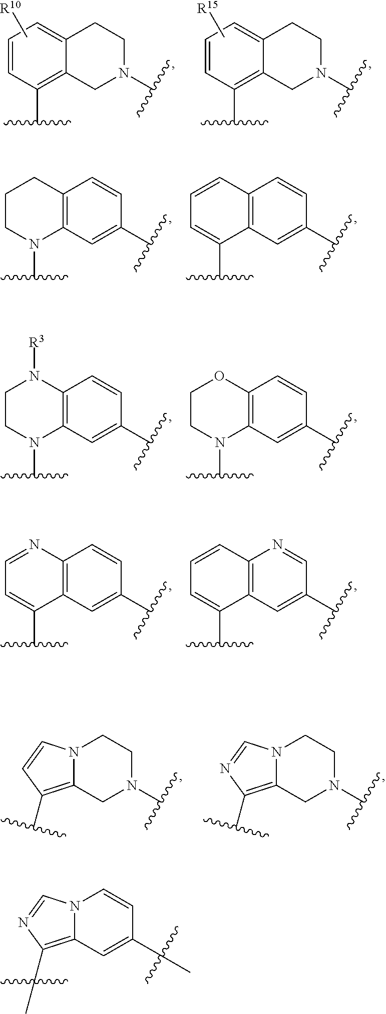

and is optionally substituted with one or more substituents independently selected from halo, hydroxy, nitro, lower alkyl, lower heteroalkyl, alkoxy, amino, cyano and halomethyl, wherein the #-N(R⁴)—R¹³—Z²— substituent of formula (IIb) is attached to Ar² at any Ar² atom capable of being substituted;
Z¹ is selected from N, CH, C-halo and C—CN;
Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O;
R¹ is selected from hydrogen, methyl, halo, halomethyl, ethyl and cyano;
R² is selected from hydrogen, methyl, halo, halomethyl and cyano;
R³ is selected from hydrogen, lower alkyl and lower heteroalkyl;
R⁴ is selected from hydrogen, lower alkyl, monocyclic cycloalkyl, monocyclic heterocyclyl, lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, lower heteroalkyl are optionally substituted with one or more halo, cyano, alkoxy, monocyclic cycloalkyl, monocyclic heterocyclyl, NC(O)CR^{6a}R^{6b}, NS(O)CR^{6a}R^{6b}, NS(O₂)CR^{6a}R^{6b}, S(O₂)CR^{6a}R^{6b} or S(O₂)NH₂ groups;
R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;
R¹⁰ is selected from cyano, OR¹⁴, SR¹⁴, SOR¹⁴, SO₂R¹⁴, SO₂NR^{14a}R^{14b}, NR^{14a}R^{14b}, NC(O)R¹⁴ and NSO₂R¹⁴;
R^{11a} and R^{11b} are each, independently of one another, selected from hydrogen, halo, methyl, ethyl, halomethyl, hydroxyl, methoxy, CN, and SCH₃;
R¹² is selected from hydrogen, halo, cyano, lower alkyl, lower heteroalkyl, cycloalkyl, or heterocyclyl, wherein the alkyl, heteroalkyl, cycloalkyl, or heterocyclyl are optionally substituted with one or more halo, cyano, alkoxy, monocyclic cycloalkyl, monocyclic heterocyclyl, NC(O)CR^{6a}R^{6b}, NS(O)CR^{6a}R^{6b}, NS(O₂)CR^{6a}R^{6b} or S(O₂)CR^{6a}R^{6b} groups;
R¹³ is selected from a bond, optionally substituted lower alkylene, optionally substituted lower heteroalkylene, optionally substituted cycloalkyl or optionally substituted heterocyclyl;
R¹⁴ is selected from hydrogen, optionally substituted lower alkyl and optionally substituted lower heteroalkyl;
R^{14a} and R^{14b} are each, independently of one another, selected from hydrogen, optionally substituted lower alkyl, optionally substituted lower heteroalkyl, or are taken together with the nitrogen atom to which they are bonded to form a monocyclic cycloalkyl or monocyclic heterocyclyl ring;
R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and
# represents a point of attachment to a linker or a hydrogen atom.

When a Bcl-xL inhibitor of structural formulae (IIa) and (IIb) is not a component of an ADC, # in formulae (IIa) and (IIb) represents the point of attachment to a hydrogen atom. When the Bcl-xL inhibitor is a component of an ADC, # in formulae (IIa) and (IIb) represents the point of attachment to a the linker. When a Bcl-xL inhibitor is a component of an ADC, the ADC may comprise one or more Bcl-xL inhibitors, which may be the same or different, but are typically the same.

In certain embodiments, Ar¹ of formula (IIa) or (IIb) is selected from

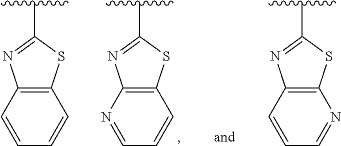

and is optionally substituted with one or more substituents independently selected from halo, cyano, methyl, and halomethyl. In particular embodiments, Ar¹ is

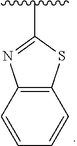 [SMILES: *c1nc2ccccc2s1]

In particular embodiments, Ar¹ is unsubstituted.

In all embodiments, the #-N(R⁴)—R¹³—Z^{2b}— substituent of formula (IIb) is attached to Ar² at any Ar² atom capable of being substituted.

In certain embodiments, Ar² of formula (IIa) or (IIb) is

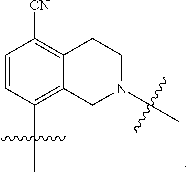 [SMILES: *c1ccc(C#N)c2c1CN(*)CC2]

In certain embodiments, Ar² of formula (IIa) or (IIb) is

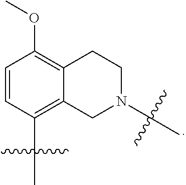 [SMILES: *c1ccc(OC)c2c1CN(*)CC2]

In certain embodiments, Ar² of formula (IIa) or (IIb) is

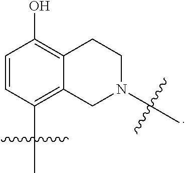 [SMILES: *c1ccc(O)c2c1CN(*)CC2]

In certain embodiments, Ar² of formula (IIa) or (IIb) is

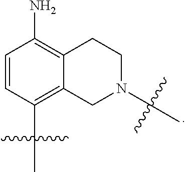 [SMILES: *c1ccc(N)c2c1CN(*)CC2]

In certain embodiments, Ar² of formula (IIa) or (IIb) is

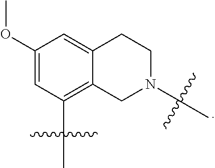 [SMILES: *c1cc(OC)cc2c1CN(*)CC2]

In certain embodiments, Ar² of formula (IIa) or (Ib) is

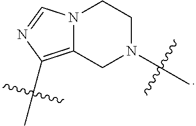 [SMILES: *c1ncn2c1CN(*)CC2]

In certain embodiments, Ar² of formula (IIa) or (IIb) is

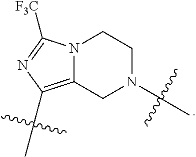 [SMILES: *c1nc(C(F)(F)F)n2c1CN(*)CC2]

In certain embodiments, Ar² of formula (IIa) or (IIb) is

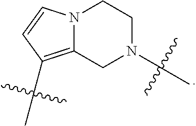 [SMILES: *c1ccn2c1CN(*)CC2]

In certain embodiments, Ar² of formula (IIa) or (IIb) is

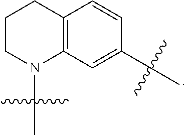 [SMILES: *c1ccc2c(c1)N(*)CCC2]

In certain embodiments, Ar² of formula (IIa) or (IIb) is

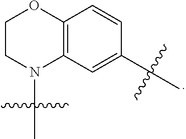 [SMILES: *c1ccc2c(c1)N(*)CCO2]

In certain embodiments, Ar² of formula (IIa) or (IIb) is

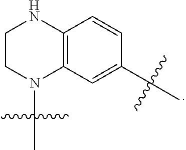 [SMILES: *c1ccc2c(c1)N(*)CCN2]

In certain embodiments, Ar² of formula (IIa) or (IIb) is

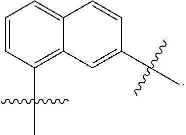 [SMILES: *c1ccc2cccc(*)c2c1]

In certain embodiments, Ar² of formula (IIa) or (IIb) is

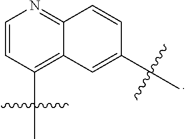 [SMILES: *c1ccc2nccc(*)c2c1]

In certain embodiments, Ar² of formula (IIa) or (IIb) is

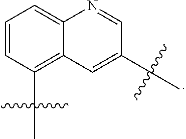 [SMILES: *c1cnc2cccc(*)c2c1]

In certain embodiments, Ar² of formula (IIa) or (IIb) is

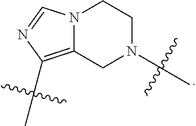 [SMILES: *c1ncn2c1CN(*)CC2]

In certain embodiments, Ar² of formula (IIa) or (IIb) is

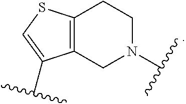 [SMILES: *c1csc2c1CN(*)CC2]

In certain embodiments, Ar² of formula (IIa) or (IIb) is

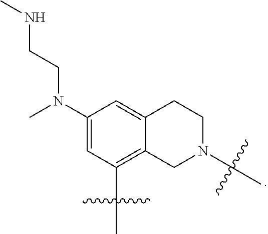 [SMILES: *c1cc(N(C)CCNC)cc2c1CN(*)CC2]

In certain embodiments, Ar² of formula (IIa) or (IIb) is

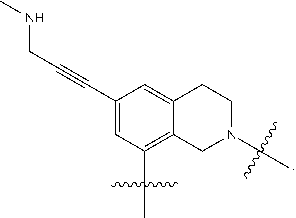 [SMILES: *c1cc(C#CCNC)cc2c1CN(*)CC2]

In certain embodiments, Ar² of formula (Ia) or (IIb) is

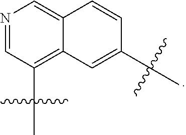 [SMILES: *c1ccc2cncc(*)c2c1]

In certain embodiments, Ar² of formula (IIa) or (IIb) is

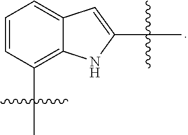 [SMILES: *c1cc2cccc(*)c2[nH]1]

In certain embodiments, Ar² of formula (IIa) or (IIb) is

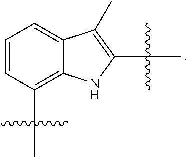 [SMILES: *c1[nH]c2c(*)cccc2c1C]

In certain embodiments, Ar² of formula (IIa) or (IIb) is

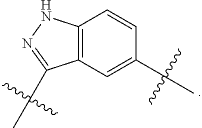 [SMILES: *c1ccc2[nH]nc(*)c2c1]

In certain embodiments, Ar² of formula (IIa) or (IIb) is

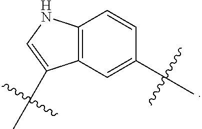 [SMILES: *c1ccc2[nH]cc(*)c2c1]

In certain embodiments, Ar² of formula (IIa) or (IIb) is

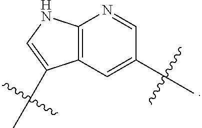 [SMILES: *c1cnc2[nH]cc(*)c2c1]

In certain embodiments, Ar² of formula (IIa) or (IIb) is

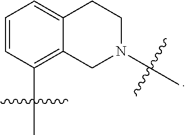 [SMILES: *c1cccc2c1CN(*)CC2]

In certain embodiments, Ar² of formula (IIa) is unsubstituted.

In certain embodiments, Ar² of formula (IIa) or (IIb) is

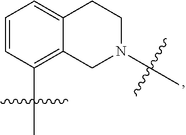 [SMILES: *c1cccc2c1CN(*)CC2]

which is substituted at the 5-position with a group selected from hydroxyl, alkoxy, and cyano.

In certain embodiments, Z¹ of formula (IIa) or (IIb) is N.

In certain embodiments, R¹ of formula (IIa) or (IIb) is selected from methyl and chloro.

In certain embodiments, R² of formula (IIa) or (IIb) is selected from hydrogen and methyl. In particular embodiments, R² is hydrogen.

In certain embodiments, R⁴ of formula (IIa) or (IIb) is methyl.

In certain embodiments, R⁴ of formula (IIa) or (Ib) is (CH₂)₂OCH₃.

In certain embodiments, R⁴ of formula (IIa) or (IIb) is hydrogen.

In certain embodiments, R⁴ of formula (IIa) or (IIb) is monocyclic heterocyclyl, wherein the monocyclic heterocycloalkyl is substituted with one S(O₂)CH₃.

In certain embodiments, R⁴ of formula (IIa) or (IIb) is lower alkyl, wherein the lower alkyl is substituted with C(O)NH₂.

In certain embodiments, R⁴ of formula (IIa) or (IIb) is lower alkyl, wherein the lower alkyl is substituted with S(O₂)NH₂.

In certain embodiments, R⁴ of formula (IIa) or (IIb) is lower alkyl, wherein the lower alkyl is substituted with hydroxy.

In certain embodiments, R⁴ of formula (IIa) or (IIb) is lower alkyl, wherein the lower alkyl is substituted with C(O)N(CH₃)₂.

In certain embodiments, R⁴ of formula (IIa) or (IIb) is lower alkyl, wherein the lower alkyl is substituted with C(O)NHCH₃.

In certain embodiments, R^{11a} and R^{11b} of formula (IIa) or (IIb) are the same. In a particular embodiment, R^{11a} and R^{11b} are each methyl. In another embodiment, R^{11a} and R^{11b} are each ethyl. In another embodiment, R^{11a} and R^{11b} are each methoxy.

In certain embodiments, R^{11a} and R^{11b} of formula (IIa) or (IIb) are independently selected from F, Br and Cl.

Certain embodiments pertain to a compound of formula (IIa). In certain embodiments, Z^{2a} of formula (IIa) is O.

In certain embodiments, Z^{2a} of formula (IIa) is methylene or O.

In certain embodiments, Z^{2a} of formula (IIa) is S.

In certain embodiments, Z^{2a} of formula (IIa) is methylene.

In certain embodiments, Z^{2a} of formula (IIa) is NR⁶. In some such embodiments R⁶ is methyl.

In certain embodiments, Z^{2a} of formula (IIa) is NR⁶C(O). In some such embodiments R⁶ is hydrogen.

In certain embodiments, Z^{2a} of formula (IIa) is O, R¹³ is ethylene, and R⁴ lower alkyl.

In certain embodiments, Z^{2a} of formula (IIa) is O, R¹³ is ethylene, and R⁴ is methyl.

In certain embodiments, Z^{2a} of formula (IIa) is O, R¹³ is ethylene, and R⁴ is hydrogen.

In certain embodiments, Z^{2a} of formula (IIa) is NR⁶C(O), R⁶ is hydrogen, R¹³ is methylene, and R⁴ is hydrogen.

In certain embodiments, Z^{2a} of formula (IIa) is S, R¹³ is ethylene, and R⁴ is hydrogen.

In certain embodiments, Z^{2a} of formula (IIa) is CH₂, R³ is ethylene, and R⁴ is hydrogen.

In certain embodiments, the group R¹³ in formula (IIa) is ethylene. In some such embodiments Z^{2a} is O.

In certain embodiments, the group R¹³ in formula (IIa) is propylene. In some such embodiments Z^{2a} is O.

In certain embodiments, the group R¹³ in formula (IIa) is selected from (CH₂)₂O(CH₂)₂, (CH₂)₃O(CH₂)₂, (CH₂)₂O(CH₂)₃ and (CH₂)₃O(CH₂)₃. In some such embodiments Z^{2a} is O.

In certain embodiments, the group R¹³ in formula (IIa) is selected from (CH₂)₂(SO₂)(CH₂)₂ (CH₂)₃(SO₂)(CH₂)₂, (CH₂)₂(SO₂)(CH₂)₃ and (CH₂)₃(SO₂)(CH₂)₃. In some such embodiments Z^{2a} is O.

In certain embodiments, the group R¹³ in formula (IIa) is selected from (CH₂)₂(SO₂)(CH)₂, (CH₂)₂(SO)(CH₂)₃, (CH₂)₃(SO)(CH₂)₂ and (CH₂)₃(SO)(CH₂)₃. In some such embodiments Z^{2a} is O.

In certain embodiments, the group R^{—}in formula (IIa) is selected from (CH₂)₂S(CH₂)₂, (CH₂)₂S(CH₂)₃, (CH₂)₃S(CH₂)₂ and (CH₂)₃S(CH₂)₃. In some such embodiments Z^{2a} is O.

In certain embodiments, the group

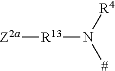

in formula (IIa) is

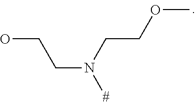

In certain embodiments, the group

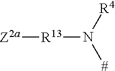

in formula (IIa) is

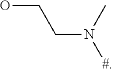

In certain embodiments, the group

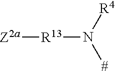

in formula (IIa) is

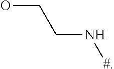

In certain embodiments, the group

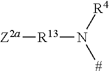

in formula (IIa) is

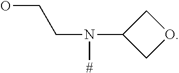

In certain embodiments, the group

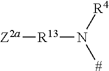

is selected from

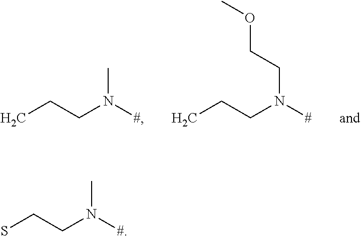

In certain embodiments, the group

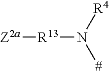

in formula (IIa) is

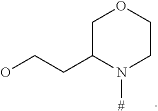

In certain embodiments, the group

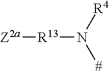

in formula (IIa) is selected from

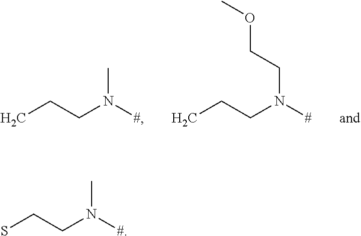

In certain embodiments, the group

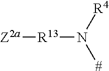

in formula (IIa) is

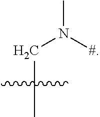

In certain embodiments, the group

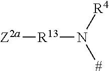

in formula (IIa) is

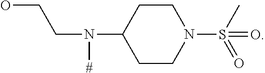

In certain embodiments, the group

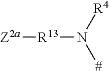

in formula (IIa) is

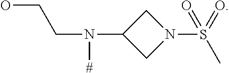

In certain embodiments, the group

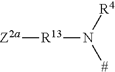

in formula (IIa) is

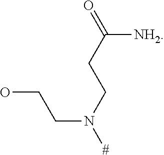

In certain embodiments, the group

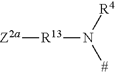

in formula (IIa) is

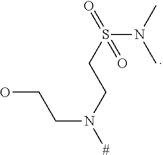

In certain embodiments, the group

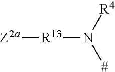

in formula (IIa) is

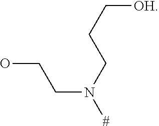

In certain embodiments, the group

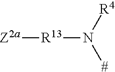

in formula (IIa) is

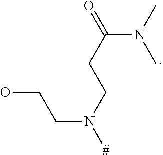

In certain embodiments, the group

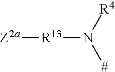

in formula (IIa) is

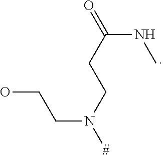

In certain embodiments, the group Z^{2b} in formula (IIb) is NR⁶. In some such embodiments R⁶ is methyl.

In certain embodiments, the group Z^{2b} in formula (IIb) is NR⁶ and R¹³ is ethylene. In some such embodiments R⁶ is methyl.

In certain embodiments, the group Z^{2b} in formula (IIb) is O and R¹³ is ethylene. In some such embodiments R⁴ is methyl.

In certain embodiments, the group Z^{2b} in formula (IIb) is NR⁶, wherein the R⁶ group is taken together with an atom of R¹³ to form a ring having between 4 and 6 atoms. In some such embodiments the ring is a five membered ring.

In certain embodiments, the group Z^{2b} in formula (IIb) is methylene and the group R¹³ is methylene.

In certain embodiments, the group Z^{2b} in formula (IIb) is methylene and the group R¹³ is a bond.

In certain embodiments, the group Z^{2b} in formula (IIb) is oxygen and the group R¹³ is selected from (CH₂)₂O(CH₂)₂, (CH₂)₃O(CH₂)₂, (CH₂)₂O(CH₂)₃ and (CH₂)₃O(CH₂)₃. In some such embodiments R⁴ is methyl.

In certain embodiments, the group Z^{2c} in formula (IIb) is a bond and R¹² is OH.

In certain embodiments, the group Z^{2c} in formula (IIb) is a bond and R¹² is selected from F, Cl, Br and I.

In certain embodiments, the group Z^{2c} in formula (IIb) is a bond and R¹² is lower alkyl. In some such embodiments R¹² is methyl.

In certain embodiments, the group Z^{2c} in formula (IIb) is O and R¹² is a lower heteroalkyl. In some such embodiments R¹² is O(CH₂)₂OCH₃.

In certain embodiments, the group Z^{2c} in formula (IIb) is O and R¹² is a lower alkyl. In particular embodiments R¹² is methyl.

In certain embodiments, the group Z^{2c} in formula (IIb) is S and R¹² is a lower alkyl. In some such embodiments R¹² is methyl.

Exemplary Bcl-xL inhibitors according to structural formulae (IIa)-(IIb) that may be used in the methods described herein in unconjugated form and/or included in the ADCs described herein include the following compounds, and/or salts thereof:

| Appln Ex. No. | Inhibitory Compound |
|---|---|
| 1.1 | W3.01 |
| 1.2 | W3.02 |
| 1.3 | W3.03 |
| 1.4 | W3.04 |
| 1.5 | W3.05 |
| 1.6 | W3.06 |
| 1.7 | W3.07 |
| 1.8 | W3.08 |
| 1.9 | W3.09 |
| 1.10 | W3.10 |
| 1.11 | W3.11 |
| 1.12 | W3.12 |
| 1.13 | W3.13 |
| 1.14 | W3.14 |
| 1.15 | W3.15 |
| 1.16 | W3.16 |
| 1.17 | W3.17 |
| 1.18 | W3.18 |
| 1.19 | W3.19 |
| 1.20 | W3.20 |
| 1.21 | W3.21 |
| 1.22 | W3.22 |
| 1.23 | W3.23 |
| 1.24 | W3.24 |
| 1.25 | W3.25 |
| 1.26 | W3.26 |
| 1.27 | W3.27 |
| 1.28 | W3.28 |
| 1.29 | W3.29 |
| 1.30 | W3.30 |
| 1.31 | W3.31 |
| 1.32 | W3.32 |
| 1.33 | W3.33 |
| 1.34 | W3.34 |
| 1.35 | W3.35 |
| 1.36 | W3.36 |
| 1.37 | W3.37 |
| 1.38 | W3.38 |
| 1.39 | W3.39 |
| 1.40 | W3.40 |
| 1.41 | W3.41 |
| 1.42 | W3.42 |
| 1.43 | W3.43 |

In certain embodiments, the Bcl-xL inhibitor is selected from the group consisting of W3.01, W3.02, W3.03, W3.04, W3.05, W3.06, W3.07, W3.08, W3.09, W3.10, W3.11, W3.12, W3.13, W3.14, W3.15, W3.16, W3.17, W3.18, W3.19, W3.20, W3.21, W3.22, W3.23, W3.24, W3.25, W3.26, W3.27, W3.28, W3.29, W3.30, W3.31, W3.32, W3.33, W3.34, W3.35, W3.36, W3.37, W3.38, W3.39, W3.40, W3.41, W3.42, W3.43, and pharmaceutically acceptable salts thereof.

In certain embodiments, the ADC, or a pharmaceutically acceptable salt thereof, comprises a drug linked to an antibody by way of a linker, wherein the drug is a Bcl-xL inhibitor selected from the group consisting of W3.01, W3.02, W3.03, W3.04, W3.05, W3.06, W3.07, W3.08, W3.09, W3.10, W3.11, W3.12, W3.13, W3.14, W3.15, W3.16, W3.17, W3.18, W3.19, W3.20, W3.21, W3.22, W3.23, W3.24, W3.25, W3.26, W3.27, W3.28, W3.29, W3.30, W3.31, W3.32, W3.33, W3.34, W3.35, W3.36, W3.37, W3.38, W3.39, W3.40, W3.41, W3.42, W3.43.

The Bcl-xL inhibitors bind to and inhibit anti-apoptotic Bcl-xL proteins, inducing apoptosis. The ability of specific Bcl-xL inhibitors according to structural formulae (IIa)-(IIb) to bind to and inhibit Bcl-xL activity may be confirmed in standard binding and activity assays, including, for example, the TR-FRET Bcl-xL binding assays described in Tao et al., 2014, ACS Med. Chem. Lett., 5:1088-1093. A specific TR-FRET Bcl-xL binding assay that can be used to confirm Bcl-xL binding is provided in Example 4, below. Typically, Bcl-xL inhibitors useful as inhibitors per se and in the ADCs described herein will exhibit a Kᵢ in the binding assay of Example 5 of less than about 1 nM, but may exhibit a significantly lower Kᵢ, for example a Kᵢ of less than about 1, 0.1, or even 0.01.

Bcl-xL inhibitory activity may also be confirmed in standard cell-based cytotoxicity assays, such as the FL5.12 cellular and Molt-4 cytotoxicity assays described in Tao et al., 2014, ACS Med. Chem. Lett., 5:1088-1093. A specific Molt-4 cellular cytotoxicity assay that may be used to confirm Bcl-xL inhibitory activity of specific Bcl-xL inhibitors that are able to permeate cell membranes is provided in Example 5, below. Typically, such cell-permeable Bcl-xL inhibitors will exhibit an EC₅₀ of less than about 500 nM in the Molt-4 cytotoxicity assay of Example 5, but may exhibit a significantly lower EC₅₀, for example an EC₅₀ of less than about 250, 100, 50, 20, 10 or even 5 nM.

The process of mitochondrial outer-membrane permeabilization (MOMP) is controlled by the Bcl-2 family proteins. Specifically, MOMP is promoted by the pro-apoptotic Bcl-2 family proteins Bax and Bak which, upon activation oligomerize on the outer mitochondrial membrane and form pores, leading to release of cytochrome c (cyt c). The release of cyt c triggers formulation of the apoptosome which, in turn, results in caspase activation and other events that commit the cell to undergo programmed cell death (see, Goldstein et al., 2005, Cell Death and Differentiation 12:453-462). The oligomerization action of Bax and Bak is antagonized by the anti-apoptotic Bcl-2 family members, including Bcl-2 and Bcl-xL. Bcl-xL inhibitors, in cells that depend upon Bcl-xL for survival, can cause activation of Bax and/or Bak, MOMP, release of cyt c and downstream events leading to apoptosis. The process of cyt c release can be assessed via western blot of both mitochondrial and cytosolic fractions of cytochrome c in cells and used as a proxy measurement of apoptosis in cells.

As a means of detecting Bcl-xL inhibitory activity and consequent release of cyt c, the cells can be treated with an agent that causes selective pore formation in the plasma, but not mitochondrial, membrane. Specifically, the cholesterol/phospholipid ratio is much higher in the plasma membrane than the mitochondrial membrane. As a result, short incubation with low concentrations of the cholesterol-directed detergent digitonin selectively permeabilizes the plasma membrane without significantly affecting the mitochondrial membrane. This agent forms insoluble complexes with cholesterol leading to the segregation of cholesterol from its normal phospholipid binding sites. This action, in turn, leads to the formation of holes about 40-50 Å wide in the lipid bilayer. Once the plasma membrane is permeabilized, cytosolic components able to pass over digitonin-formed holes can be washed out, including the cytochrome C that was released from mitochondria to cytosol in the apoptotic cells (Campos, 2006, Cytometry A 69(6):515-523).

Although many of the Bcl-xL inhibitors of structural formulae (IIa)-(IIb) selectively or specifically inhibit Bcl-xL over other anti-apoptotic Bcl-2 family proteins, selective and/or specific inhibition of Bcl-xL is not necessary. The Bcl-xL inhibitors and ADCs comprising the compounds may also, in addition to inhibiting Bcl-xL, inhibit one or more other anti-apoptotic Bcl-2 family proteins, such as, for example, Bcl-2. In some embodiments, the Bcl-xL inhibitors and/or ADCs are selective and/or specific for Bcl-xL. By specific or selective is meant that the particular Bcl-xL inhibitor and/or ADC binds or inhibits Bcl-xL to a greater extent than Bcl-2 under equivalent assay conditions. In specific embodiments, the Bcl-xL inhibitors and/or ADCs exhibit in the range of about 10-fold, 100-fold, or even greater specificity or selectivity for Bcl-xL than Bcl-2 in binding assays.

### 4.4. Linkers

In the ADCs described herein, the Bcl-xL inhibitors are linked to the antibody by way of linkers. The linker linking a Bcl-xL inhibitor to the antibody of an ADC may be short, long, hydrophobic, hydrophilic, flexible or rigid, or may be composed of segments that each independently have one or more of the above-mentioned properties such that the linker may include segments having different properties. The linkers may be polyvalent such that they covalently link more than one Bcl-xL inhibitor to a single site on the antibody, or monovalent such that covalently they link a single Bcl-xL inhibitor to a single site on the antibody.

As will be appreciated by skilled artisans, the linkers link the Bcl-xL inhibitors to the antibody by forming a covalent linkage to the Bcl-xL inhibitor at one location and a covalent linkage to antibody at another. The covalent linkages are formed by reaction between functional groups on the linker and functional groups on the inhibitors and antibody. As used herein, the expression “linker” is intended to include (i) unconjugated forms of the linker that include a functional group capable of covalently linking the linker to a Bcl-xL inhibitor and a functional group capable of covalently linking the linker to an antibody; (ii) partially conjugated forms of the linker that include a functional group capable of covalently linking the linker to an antibody and that is covalently linked to a Bcl-xL inhibitor, or vice versa; and (iii) fully conjugated forms of the linker that is covalently linked to both a Bcl-xL inhibitor and an antibody. In some specific embodiments of intermediate synthons and ADCs described herein, moieties comprising the functional groups on the linker and covalent linkages formed between the linker and antibody are specifically illustrated as R^{x} and LK, respectively. One embodiment pertains to an ADC formed by contacting an antibody that binds a cell surface receptor or tumor associated antigen expressed on a tumor cell with a synthon described herein under conditions in which the synthon covalently links to the antibody. One embodiment pertains to a method of making an ADC formed by contacting a synthon described herein under conditions in which the synthon covalently links to the antibody. One embodiment pertains to a method of inhibiting Bcl-xL activity in a cell that expresses Bcl-xL, comprising contacting the cell with an ADC described herein that is capable of binding the cell, under conditions in which the ADC binds the cell.

Exemplary polyvalent linkers that may be used to link many Bcl-xL inhibitors to an antibody are described, for example, in U.S. Pat. No. 8,399,512; U.S. Published Application No. 2010/0152725; U.S. Pat. Nos. 8,524,214; 8,349,308; U.S. Published Application No. 2013/189218; U.S. Published Application No. 2014/017265; WO 2014/093379; WO 2014/093394; WO 2014/093640, the contents of which are incorporated herein by reference in their entireties. For example, the Fleximer® linker technology developed by Mersana et al. has the potential to enable high-DAR ADCs with good physicochemical properties. As shown below, the Fleximer® linker technology is based on incorporating drug molecules into a solubilizing poly-acetal backbone via a sequence of ester bonds. The methodology renders highly-loaded ADCs (DAR up to 20) whilst maintaining good physicochemical properties. This methodology could be utilized with Bcl-xL inhibitors as shown in the Scheme below.

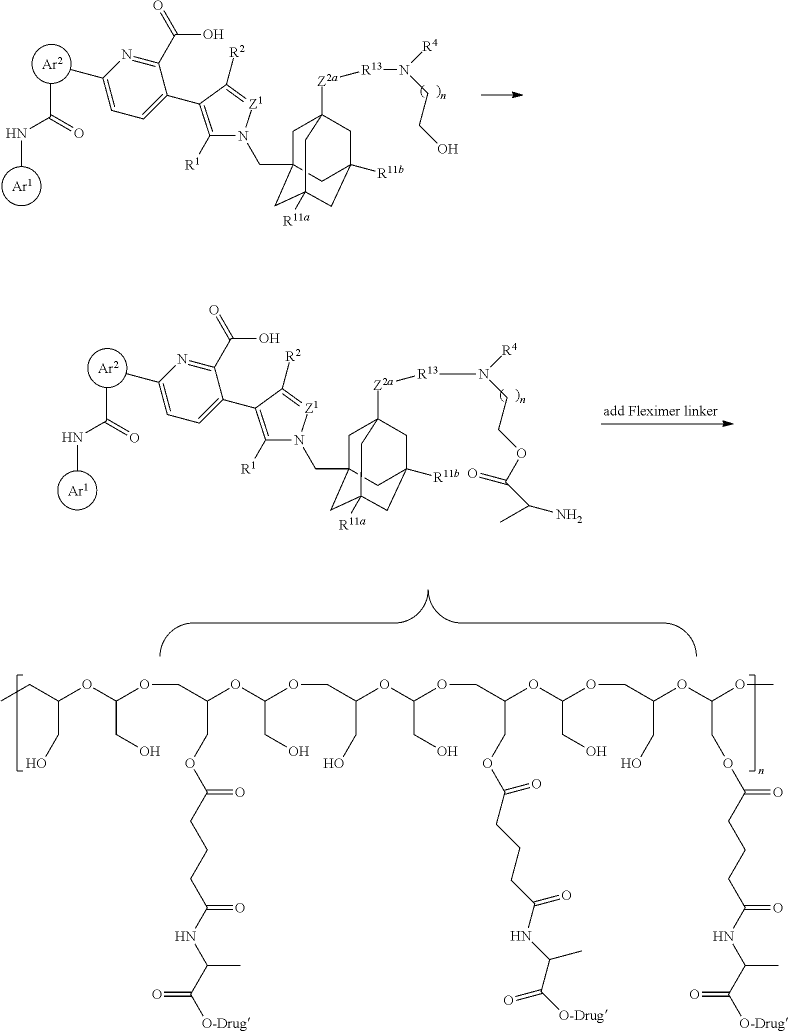

To utilize the Fleximer® linker technology depicted in the scheme above, an aliphatic alcohol can be present or introduced into the Bcl-xL inhibitor. The alcohol moiety is then conjugated to an alanine moiety, which is then synthetically incorporated into the Fleximer® linker. Liposomal processing of the ADC in vitro releases the parent alcohol-containing drug.

Additional examples of dendritic type linkers can be found in US 2006/116422; US 2005/271615; de Groot et al., (2003) Angew. Chem. Int. Ed. 42:4490-4494; Amir et al., (2003) Angew. Chem. Int. Ed. 42:4494-4499; Shamis et al., (2004) J. Am. Chem. Soc. 126:1726-1731; Sun et al., (2002) Bioorganic & Medicinal Chemistry Letters 12:2213-2215; Sun et al., (2003) Bioorganic & Medicinal Chemistry 11:1761-1768; King et al., (2002) Tetrahedron Letters 43:1987-1990.

Exemplary monovalent linkers that may be used are described, for example, in Nolting, 2013, Antibody-Drug Conjugates, Methods in Molecular Biology 1045:71-100; Kitson et al., 2013, CROs/CMOs—Chemica Oggi—Chemistry Today 31(4); 30-36; Ducry et al., 2010. Bioconjugate Chem, 21:5-13; Zhao et al., 2011, J. Med. Chem, 54:3606-3623; U.S. Pat. Nos. 7,223,837; 8,568,728; 8,535,678; and WO2004010957, the content of each of which is incorporated herein by reference in their entireties.

By way of example and not limitation, some cleavable and noncleavable linkers that may be included in the ADCs described herein are described below.

### 4.4.1.1. Cleavable Linkers

In certain embodiments, the linker selected is cleavable in vitro and in vivo. Cleavable linkers may include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is noncleavable.

In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the linker, the linker may be varied by chemical modification, e.g., substitution, allowing tuning to achieve more efficient release in the lysosome with a minimized loss in circulation.

Hydrazone-containing linkers may contain additional cleavage sites, such as additional acid-labile cleavage sites and/or enzymatically labile cleavage sites. ADCs including exemplary hydrazone-containing linkers include the following structures:

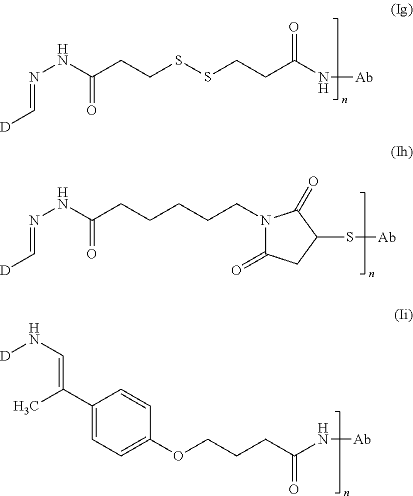

wherein D and Ab represent the drug and Ab, respectively, and n represents the number of drug-linkers linked to the antibody. In certain linkers such as linker (Ig), the linker comprises two cleavable groups—a disulfide and a hydrazone moiety. For such linkers, effective release of the unmodified free drug requires acidic pH or disulfide reduction and acidic pH. Linkers such as (Ih) and (Ii) have been shown to be effective with a single hydrazone cleavage site.

Other acid-labile groups that may be included in linkers include cis-aconityl-containing linkers. cis-Aconityl chemistry uses a carboxylic acid juxtaposed to an amide bond to accelerate amide hydrolysis under acidic conditions.

Cleavable linkers may also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonable stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 μM. Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker. e.g., use of steric hindrance adjacent to the disulfide bond.

ADCs including exemplary disulfide-containing linkers include the following structures:

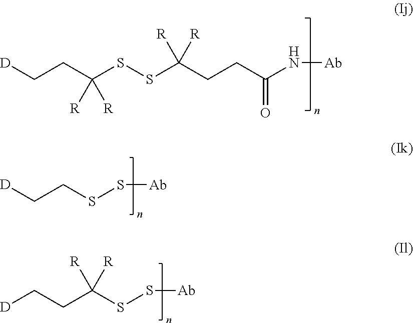

wherein D and Ab represent the drug and antibody, respectively, n represents the number of drug-linkers linked to the antibody and R is independently selected at each occurrence from hydrogen or alkyl, for example. In certain embodiments, increasing steric hindrance adjacent to the disulfide bond increases the stability of the linker. Structures such as (Ij) and (Il) show increased in vivo stability when one or more R groups is selected from a lower alkyl such as methyl.

Another type of linker that may be used is a linker that is specifically cleaved by an enzyme. In one embodiment, the linker is cleavable by a lysosomal enzyme. Such linkers are typically peptide-based or include peptidic regions that act as substrates for enzymes. Peptide based linkers tend to be more stable in plasma and extracellular millieu than chemically labile linkers. Peptide bonds generally have good serum stability, as lysosomal proteolytic enzymes have very low activity in blood due to endogenous inhibitors and the unfavorably high pH value of blood compared to lysosomes. Release of a drug from an antibody occurs specifically due to the action of lysosomal proteases, e.g., cathepsin and plasmin. These proteases may be present at elevated levels in certain tumor tissues. In certain embodiments, the linker is cleavable by a lysosomal enzyme. In certain embodiments, the linker is cleavable by a lysosomal enzyme, and the lysosomal enzyme is Cathepsin B. In certain embodiments, the linker is cleavable by a lysosomal enzyme, and the lysosomal enzyme is β-glucuronidase or β-galactosidase. In certain embodiments, the linker is cleavable by a lysosomal enzyme, and the lysosomal enzyme is β-glucuronidase. In certain embodiments, the linker is cleavable by a lysosomal enzyme, and the lysosomal enzyme is β-galactosidase.

In exemplary embodiments, the cleavable peptide is selected from tetrapeptides such as Gly-Phe-Leu-Gly, Ala-Leu-Ala-Leu or dipeptides such as Val-Cit, Val-Ala, and Phe-Lys. In certain embodiments, dipeptides are preferred over longer polypeptides due to hydrophobicity of the longer peptides.

A variety of dipeptide-based cleavable linkers useful for linking drugs such as doxorubicin, mitomycin, camptothecin, tallysomycin and auristatin/auristatin family members to antibodies have been described (see, Dubowchik et al., 1998, J. Org. Chem. 67:1866-1872; Dubowchik et al., 1998, Bioorg. Med. Chem. Lett. 8:3341-3346; Walker et al., 2002, Bioorg. Med. Chem. Let. 12:217-219; Walker et al., 2004, Bioorg. Med. Chem. Lett. 14:4323-4327; and Francisco et al., 2003, Blood 102:1458-1465, the contents of each of which are incorporated herein by reference). All of these dipeptide linkers, or modified versions of these dipeptide linkers, may be used in the ADCs described herein. Other dipeptide linkers that may be used include those found in ADCs such as Seattle Genetics' Brentuximab Vendotin SGN-35 (Adcetris™), Seattle Genetics SGN-75 (anti-CD-70, MC-monomethyl auristatin F(MMAF), Celldex Therapeutics glembatumumab (CDX-011) (anti-NMB, Val-Cit-monomethyl auristatin E(MMAE), and Cytogen PSMA-ADC (PSMA-ADC-1301) (anti-PSMA, Val-Cit-MMAE).

Enzymatically cleavable linkers may include a self-immolative spacer to spatially separate the drug from the site of enzymatic cleavage. The direct attachment of a drug to a peptide linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the linker (to give a p-amidobenzylcarbamate, PABC). The resulting prodrugs are activated upon protease-mediated cleavage, leading to a 1,6-elimination reaction releasing the unmodified drug, carbon dioxide, and remnants of the linker group. The following scheme depicts the fragmentation of p-amidobenzyl carbamate and release of the drug:

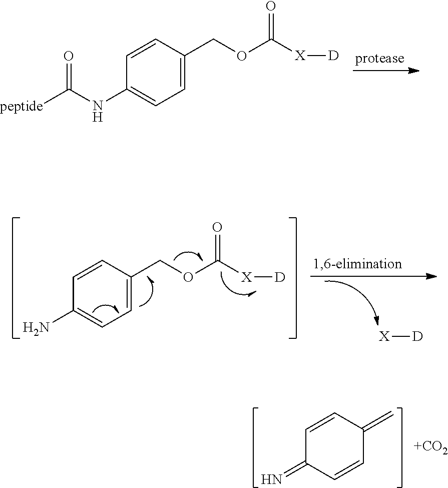

wherein X-D represents the unmodified drug.

Heterocyclic variants of this self-immolative group have also been described. See U.S. Pat. No. 7,989,434.

In certain embodiments, the enzymatically cleavable linker is a β-glucuronic acid-based linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based linkers may be used to circumvent the tendency of an ADC to undergo aggregation due to the hydrophilic nature of β-glucuronides. In certain embodiments, β-glucuronic acid-based linkers are preferred as linkers for ADCs linked to hydrophobic drugs. The following scheme depicts the release of the drug from and ADC containing a β-glucuronic acid-based linker:

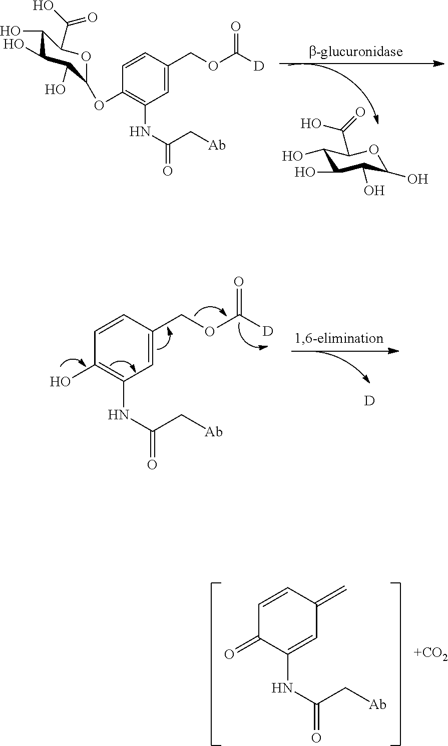

A variety of cleavable β-glucuronic acid-based linkers useful for linking drugs such as auristatins, camptothecin and doxorubicin analogues, CBI minor-groove binders, and psymberin to antibodies have been described (see. Jeffrey et al, 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255, the contents of each of which are incorporated herein by reference). All of these 1-glucuronic acid-based linkers may be used in the ADCs described herein. In certain embodiments, the enzymatically cleavable linker is a β-galactoside-based linker. β-Galactoside is present abundantly within lysosomes, while the enzyme activity outside cells is low.

Additionally, Bcl-xL inhibitors containing a phenol group can be covalently bonded to a linker through the phenolic oxygen. One such linker, described in U.S. Patent App. No. 2009/0318668, relies on a methodology in which a diamino-ethane “SpaceLink” is used in conjunction with traditional “PABO”-based self-immolative groups to deliver phenols. The cleavage of the linker is depicted schematically below using a Bcl-xL inhibitor of the disclosure.

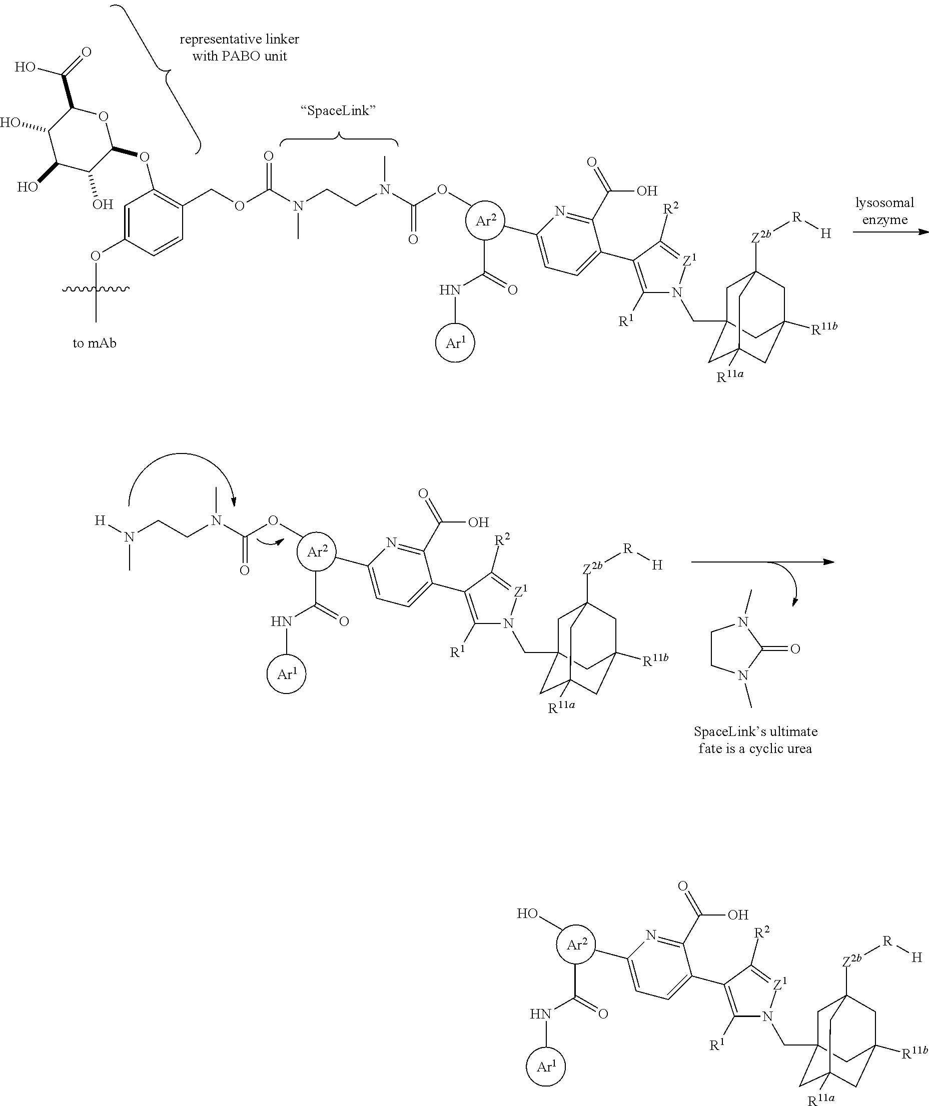

Cleavable linkers may include noncleavable portions or segments, and/or cleavable segments or portions may be included in an otherwise non-cleavable linker to render it cleavable. By way of example only, polyethylene glycol (PEG) and related polymers may include cleavable groups in the polymer backbone. For example, a polyethylene glycol or polymer linker may include one or more cleavable groups such as a disulfide, a hydrazone or a dipeptide.

Other degradable linkages that may be included in linkers include ester linkages formed by the reaction of PEG carboxylic acids or activated PEG carboxylic acids with alcohol groups on a biologically active agent, wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the linker comprises an enzymatically cleavable peptide moiety, for example, a linker comprising structural formula (IVa), (IVb), (IVc), or (Vd):

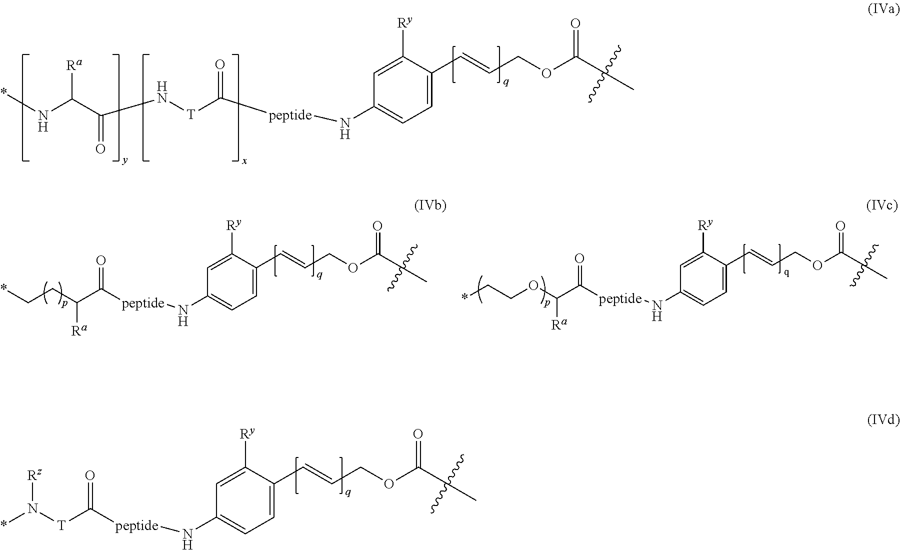

or a salt thereof, wherein:
peptide represents a peptide (illustrated N→C, wherein peptide includes the amino and carboxy “termini”) a cleavable by a lysosomal enzyme;
T represents a polymer comprising one or more ethylene glycol units or an alkylene chain, or combinations thereof;
R^{a} is selected from hydrogen, alkyl, sulfonate and methyl sulfonate;
R^{y} is hydrogen or C₁₋₄ alkyl-(O)ₜ-(C₁₋₄ alkylene)ₛ-G¹ or C₁₋₄ alkyl-(N)—[(C₁₋₄ alkylene)-G¹]₂;
R^{z} is C₁₋₄ alkyl-(O)ᵣ—(C₁₋₄ alkylene)ₛ-G²;
G¹ is SO₃H, CO₂H, PEG 4-32, or sugar moiety;
G² is SO₃H, CO₂H, or PEG 4-32 moiety;
r is 0 or 1;
s is 0 or 1;
p is an integer ranging from 0 to 5;
q is 0 or 1;
x is 0 or 1;
y is 0 or 1;
[symbol] represents the point of attachment of the linker to the Bcl-xL inhibitor; and
represents the point of attachment to the remainder of the linker.

In certain embodiments, the linker comprises an enzymatically cleavable peptide moiety, for example, a linker comprising structural formula (IVa), (IVb), (Vc), (Vd) or salts thereof.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IVa) that may be included in the ADCs described herein include the linkers illustrated below (as illustrated, the linkers include a group suitable for covalently linking the linker to an antibody):

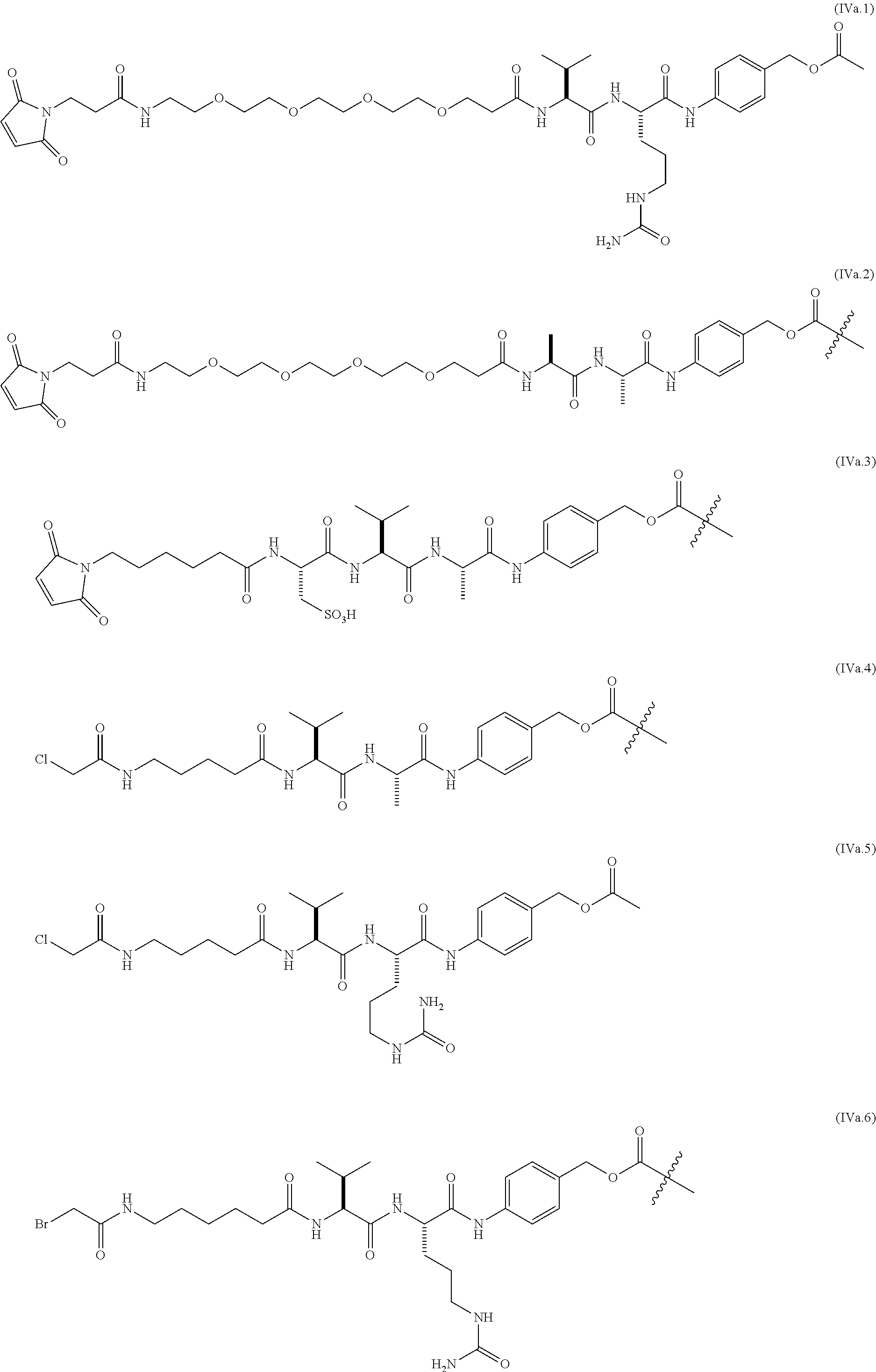

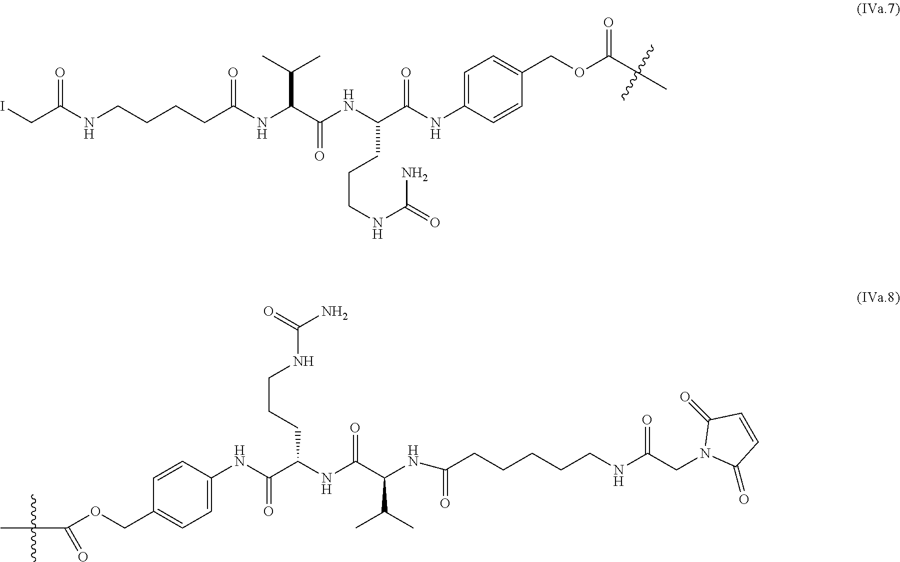

Exemplary embodiments of linkers according to structural formula (IVb), (IVc), or (IVd) that may be included in the ADCs described herein include the linkers illustrated below (as illustrated, the linkers include a group suitable for covalently linking the linker to an antibody):

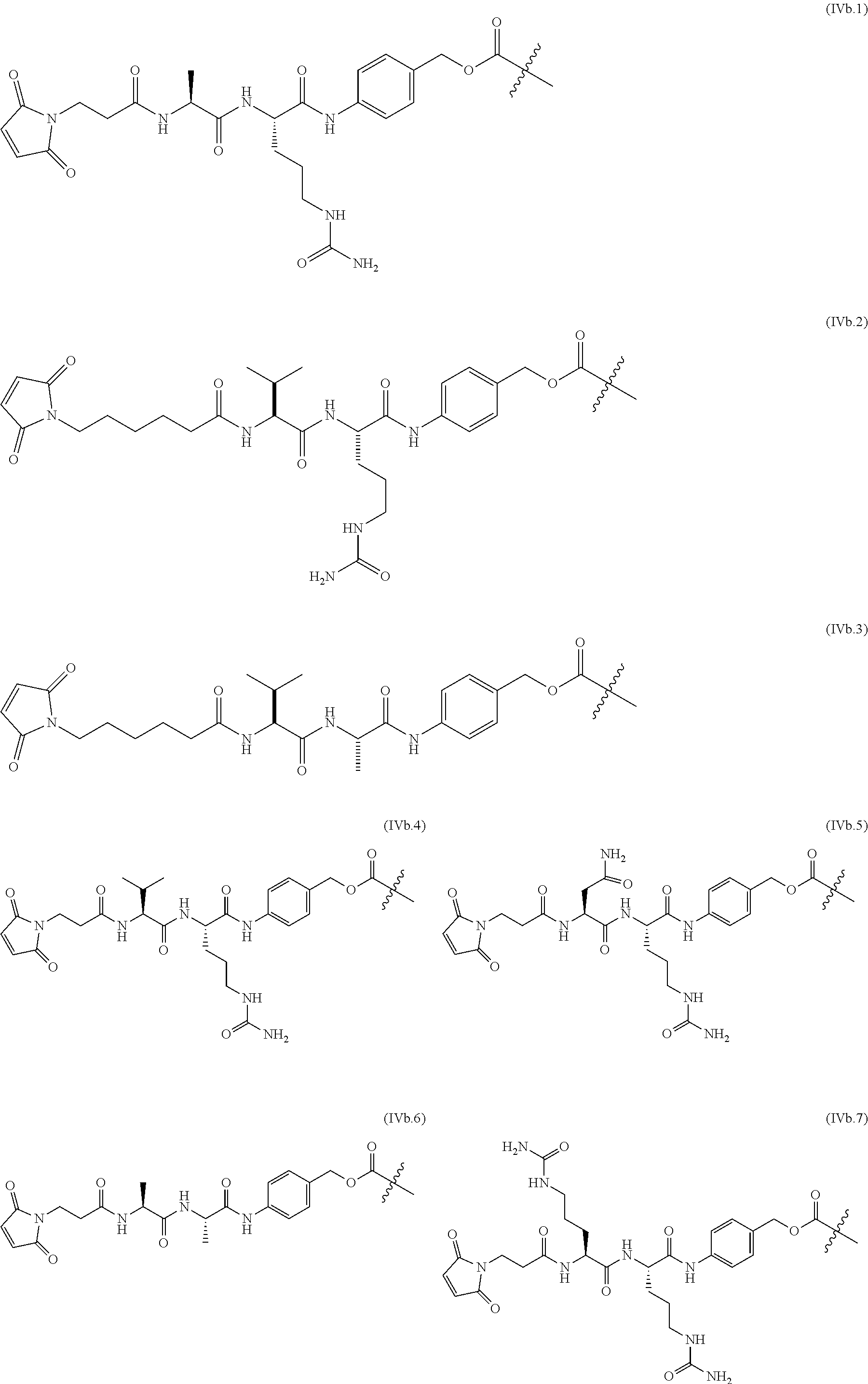

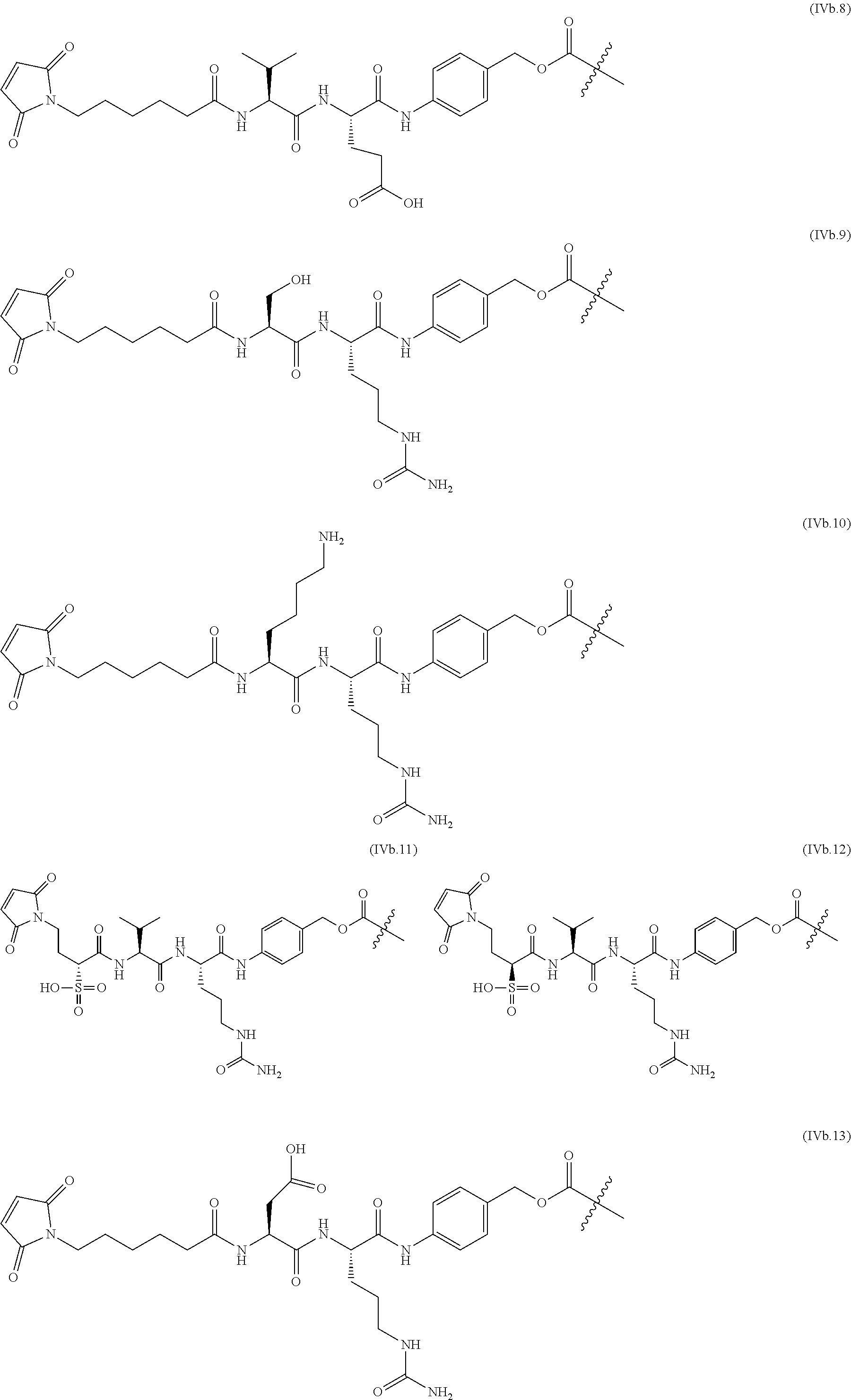

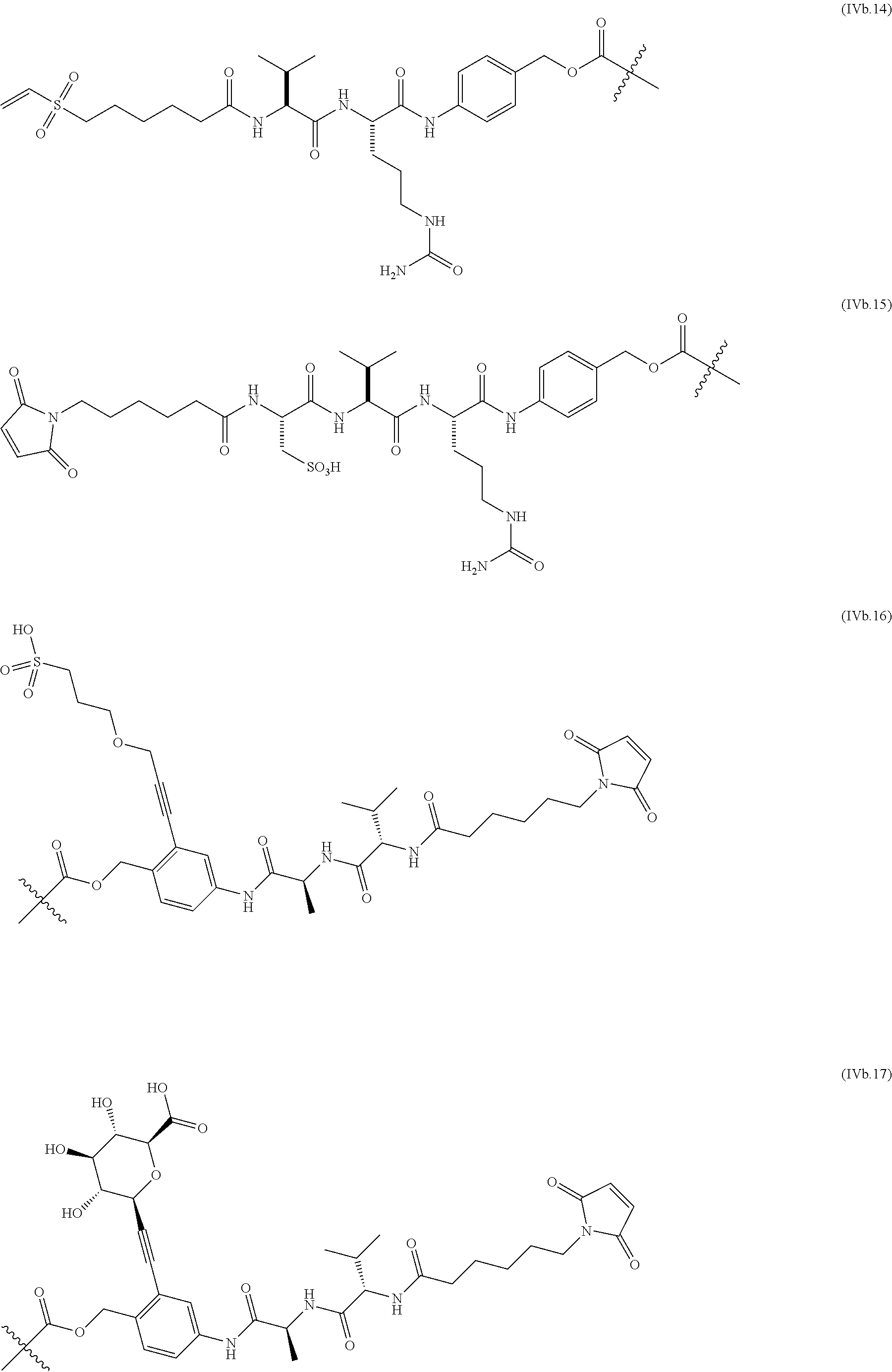 [SMILES: *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O]

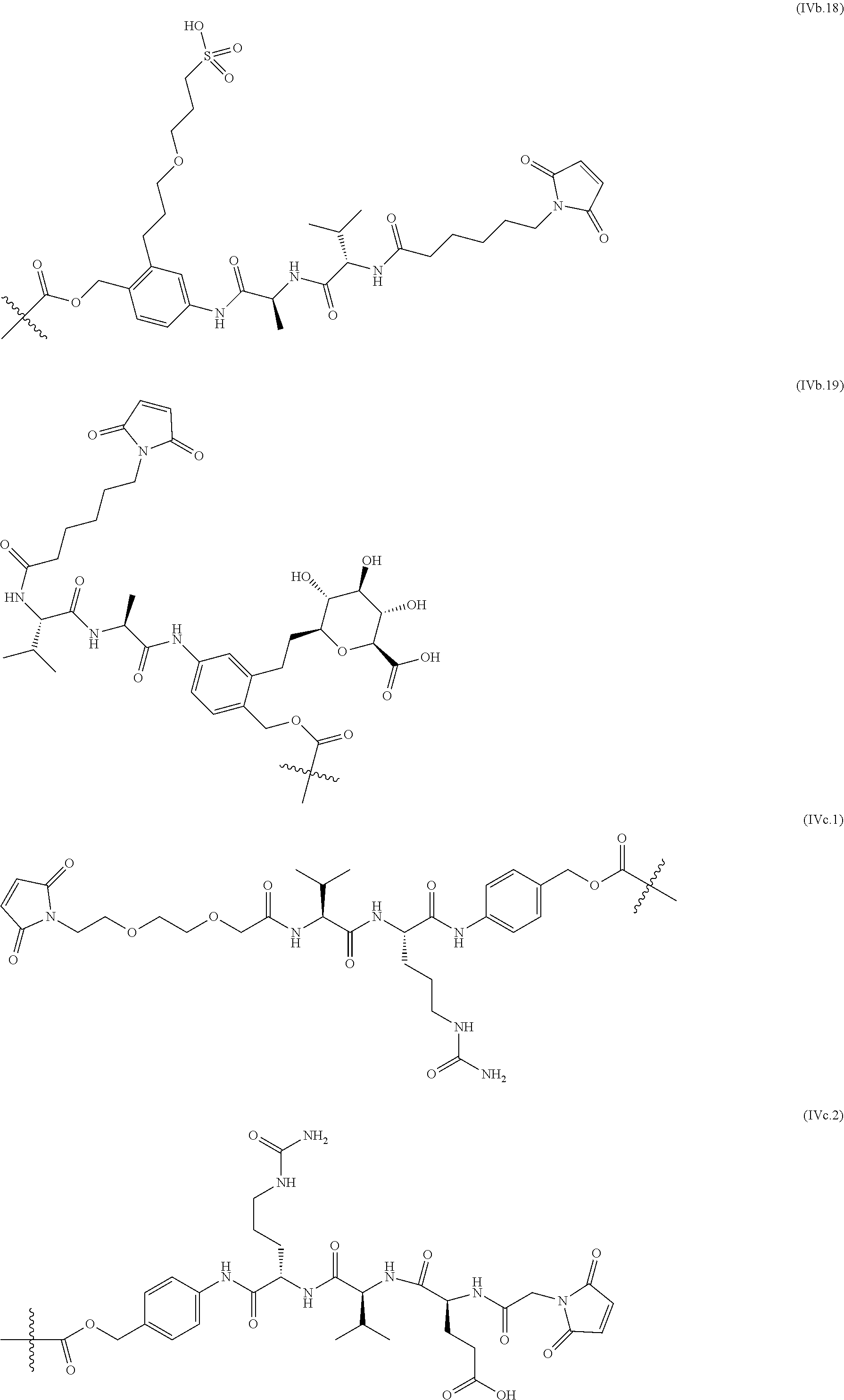 [SMILES: *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1]

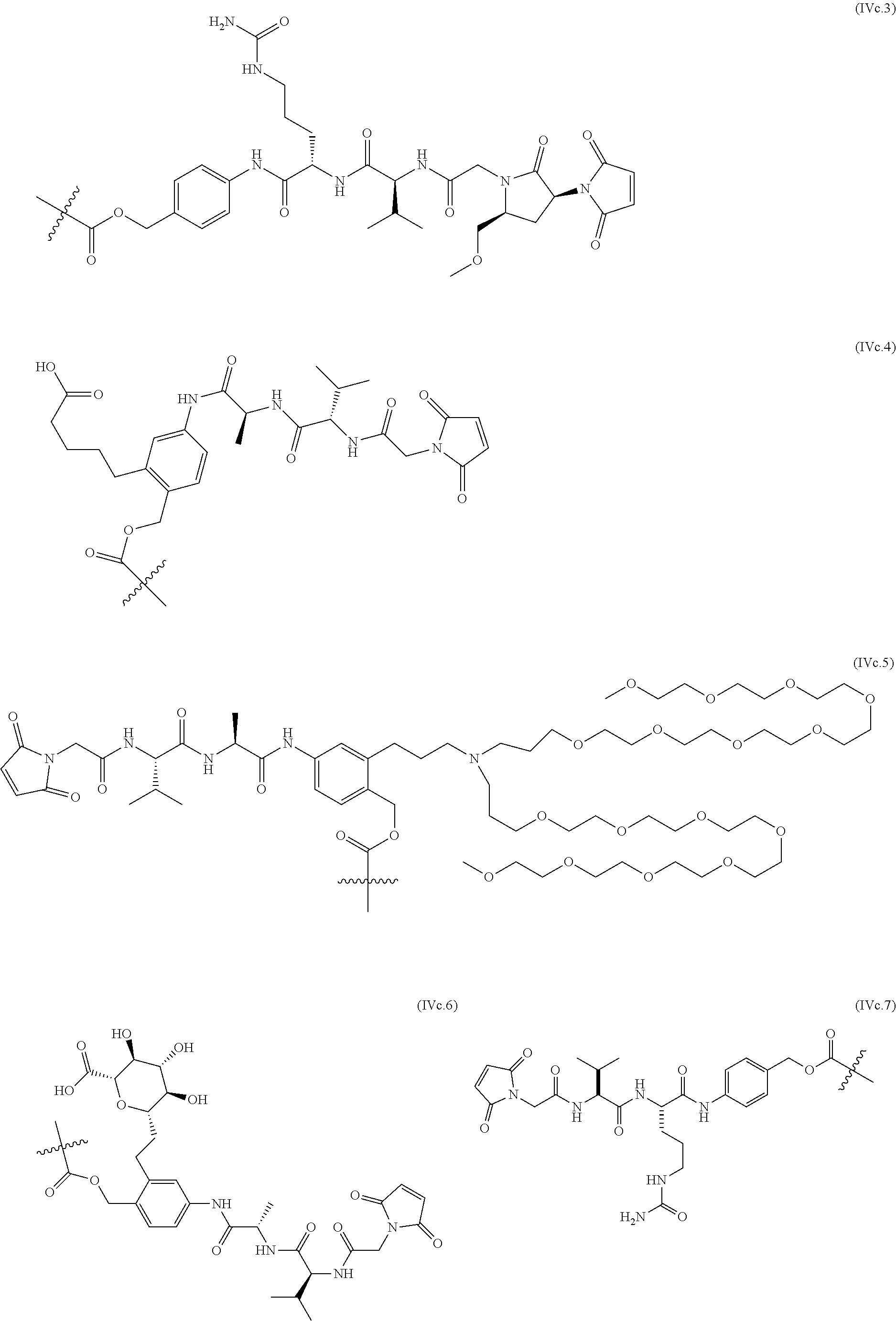

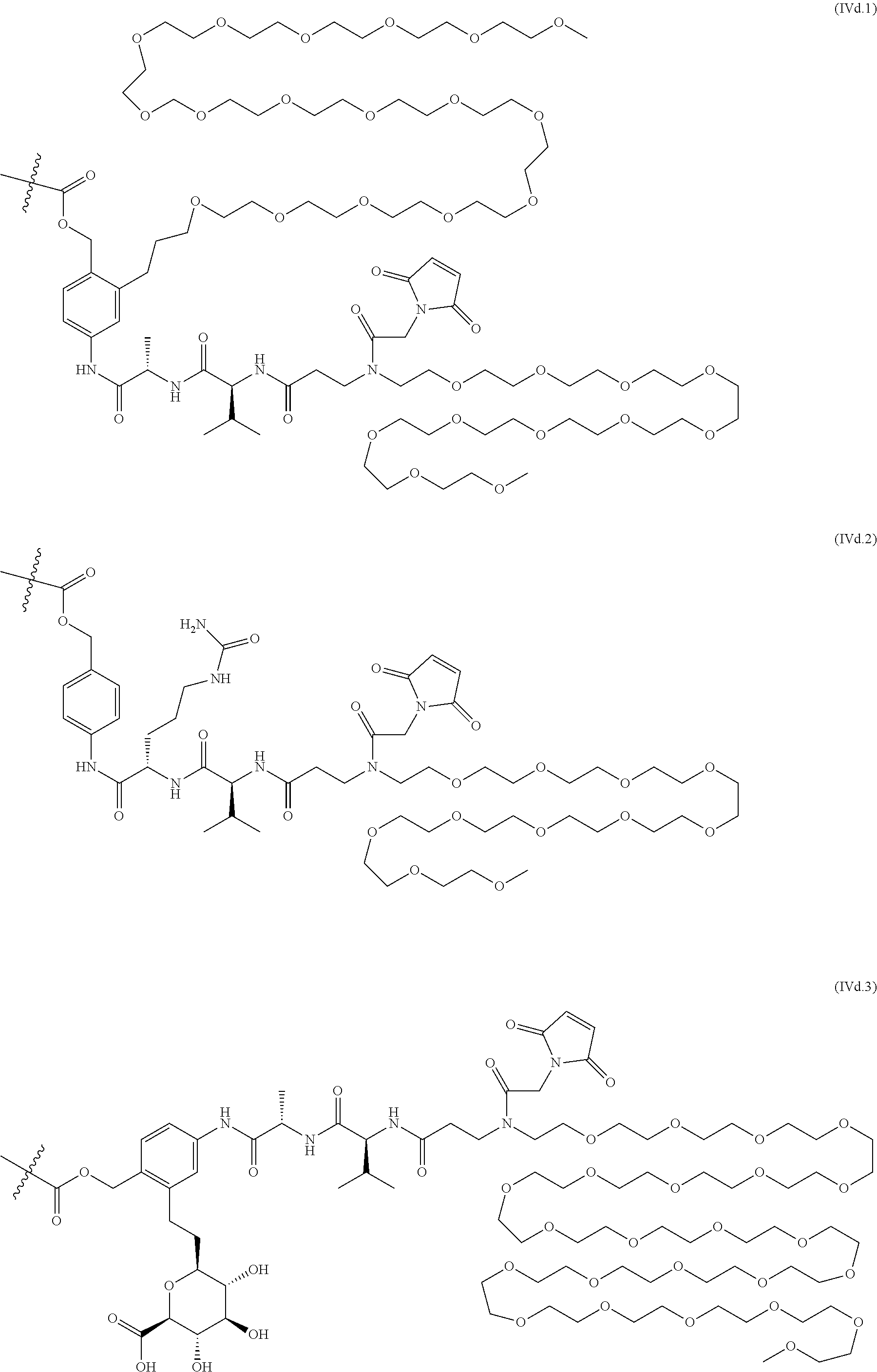

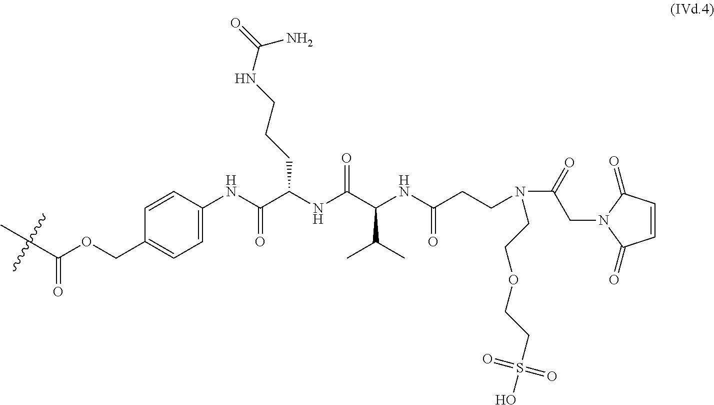 [SMILES: *C(=O)OCc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

In certain embodiments, the linker comprises an enzymatically cleavable sugar moiety, for example, a linker comprising structural formula (Va), (Vb), (Vc), (Vd), or (Ve):

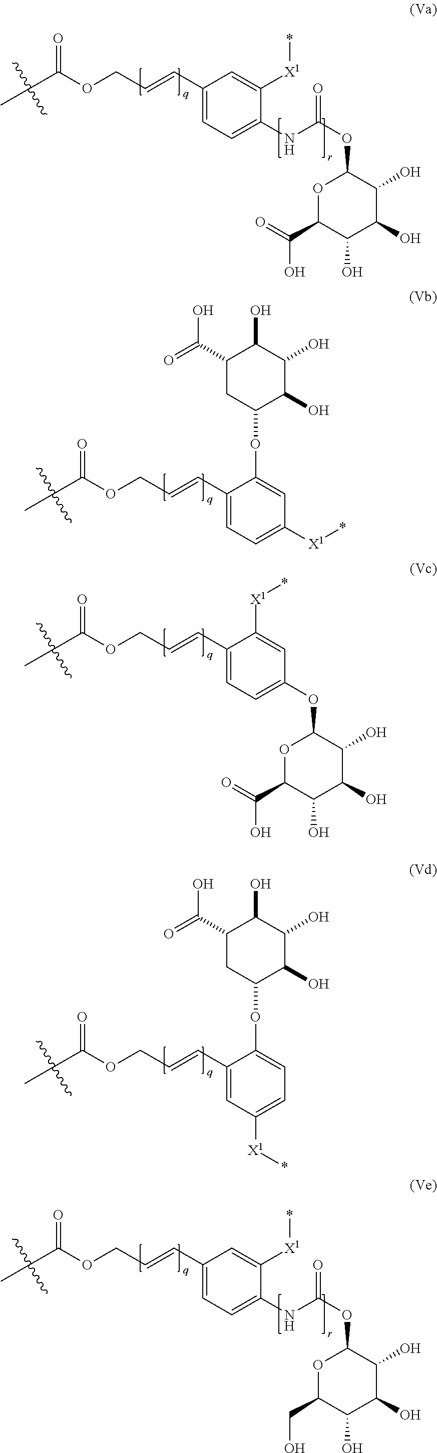

or a salt thereof, wherein:
q is 0 or 1;
r is 0 or 1;
X¹ is CH₂, O or NH;
[symbol] represents the point of attachment of the linker to the drug; and
represents the point of attachment to the remainder of the linker.

Exemplary embodiments of linkers according to structural formula (Va) that may be included in the ADCs described herein include the linkers illustrated below (as illustrated, the linkers include a group suitable for covalently linking the linker to an antibody):

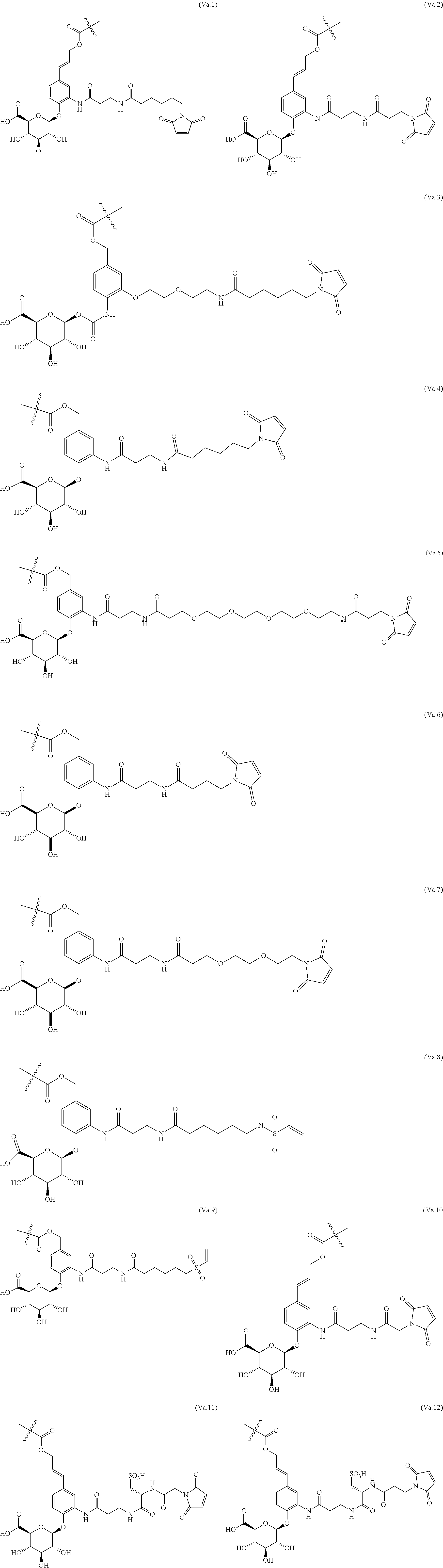

Exemplary embodiments of linkers according to structural formula (Vb) that may be included in the ADCs described herein include the linkers illustrated below (as illustrated, the linkers include a group suitable for covalently linking the linker to an antibody):

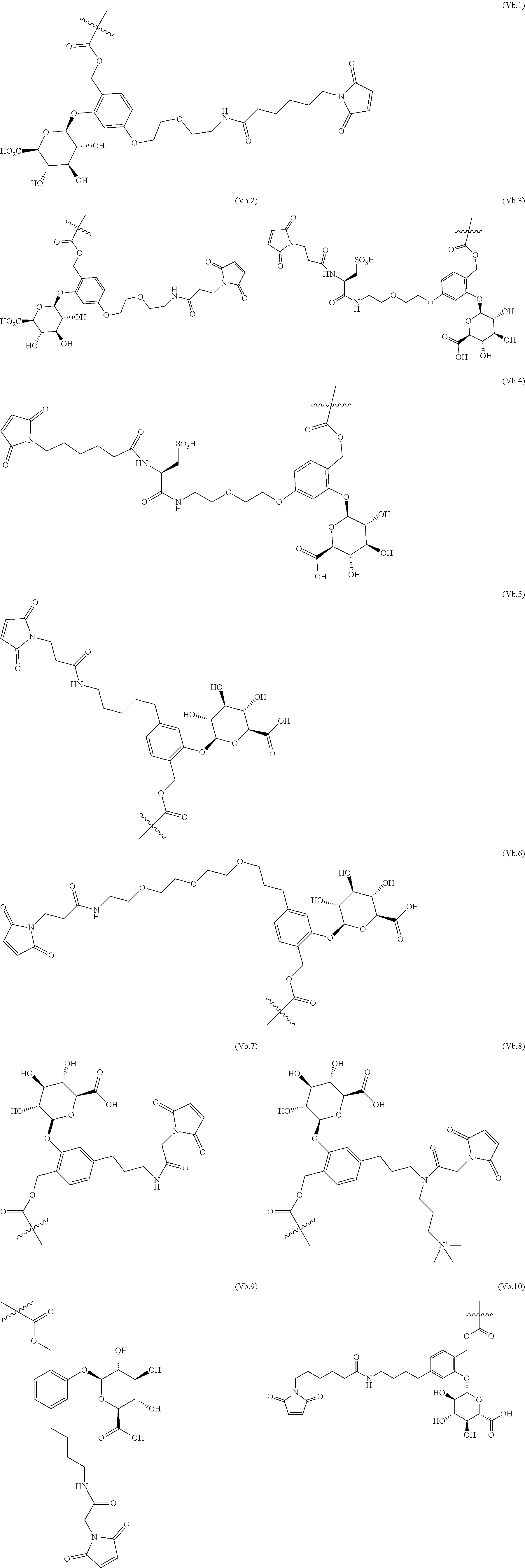

Exemplary embodiments of linkers according to structural formula (Vc) that may be included in the ADCs described herein include the linkers illustrated below (as illustrated, the linkers include a group suitable for covalently linking the linker to an antibody):

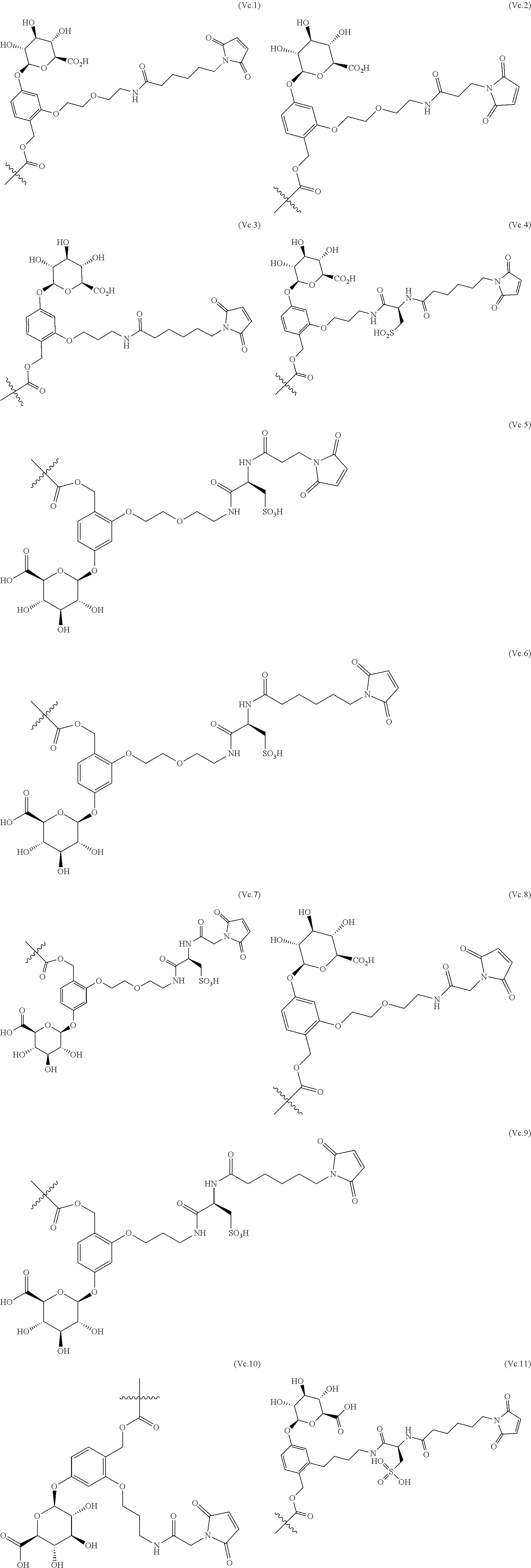

Exemplary embodiments of linkers according to structural formula (Vd) that may be included in the ADCs described herein include the linkers illustrated below (as illustrated, the linkers include a group suitable for covalently linking the linker to an antibody):

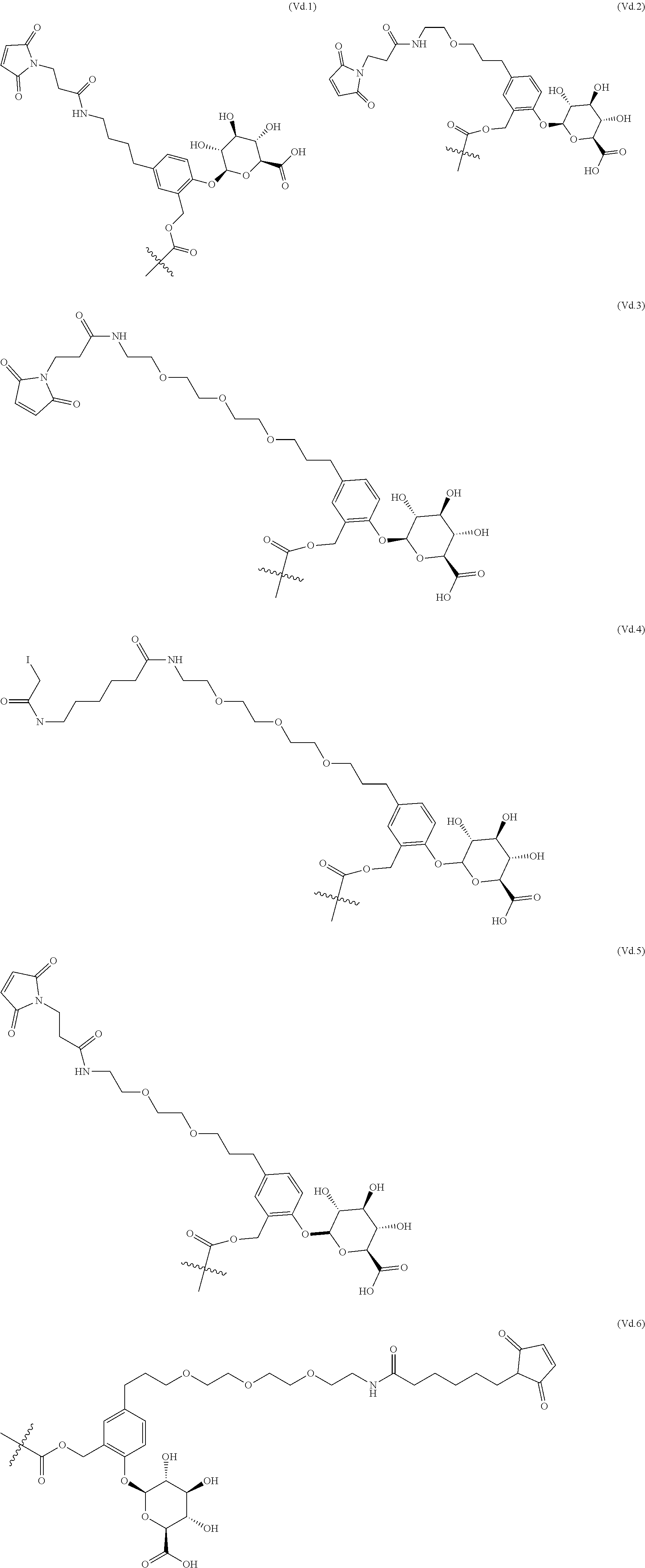

Exemplary embodiments of linkers according to structural formula (Ve) that may be included in the ADCs described herein include the linkers illustrated below (as illustrated, the linkers include a group suitable for covalently linking the linker to an antibody):

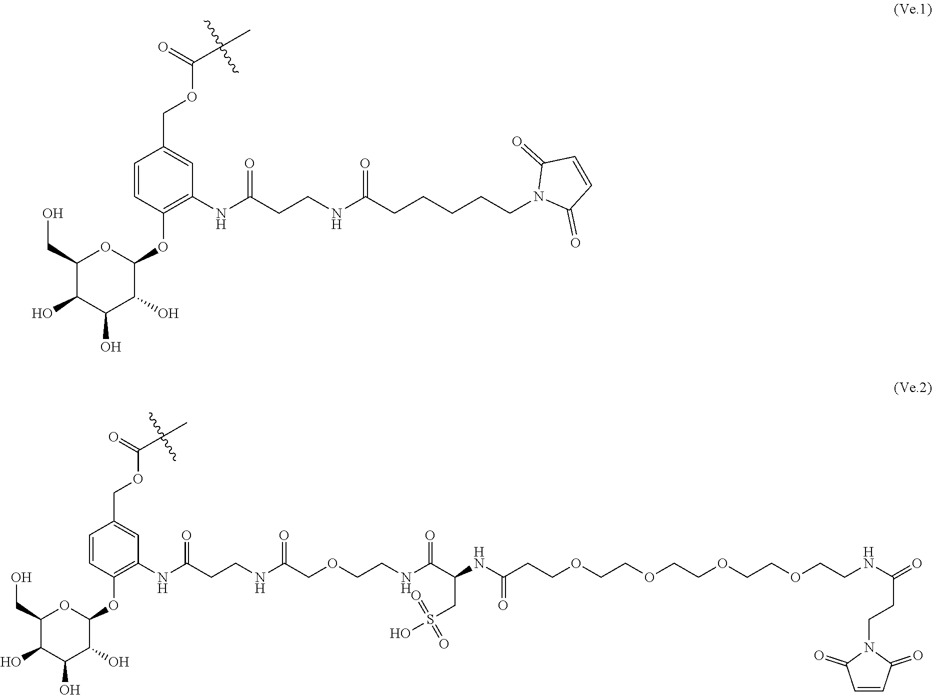 [SMILES: *C(=O)OCc1ccc(O[C@@H]2O[C@H](CO)[C@H](O)[C@H](O)[C@H]2O)c(NC(=O)CCNC(=O)CCCCCN2C(=O)C=CC2=O)c1 ; *C(=O)OCc1ccc(O[C@@H]2O[C@H](CO)[C@H](O)[C@H](O)[C@H]2O)c(NC(=O)CCNC(=O)COCCNC(=O)[C@H](CS(=O)(=O)O)NC(=O)CCOCCOCCOCCOCCNC(=O)CCN2C(=O)C=CC2=O)c1]

### 4.4.1.2. Non-Cleavable Linkers

Although cleavable linkers may provide certain advantages, the linkers comprising the ADC described herein need not be cleavable. For noncleavable linkers, the drug release does not depend on the differential properties between the plasma and some cytoplasmic compartments. The release of the drug is postulated to occur after internalization of the ADC via antigen-mediated endocytosis and delivery to lysosomal compartment, where the antibody is degraded to the level of amino acids through intracellular proteolytic degradation. This process releases a drug derivative, which is formed by the drug, the linker, and the amino acid residue to which the linker was covalently attached. The amino-acid drug metabolites from conjugates with noncleavable linkers are more hydrophilic and generally less membrane permeable, which leads to less bystander effects and less nonspecific toxicities compared to conjugates with a cleavable linker. In general, ADCs with noncleavable linkers have greater stability in circulation than ADCs with cleavable linkers. Non-cleavable linkers may be alkylene chains, or maybe polymeric in natures, such as, for example, based upon polyalkylene glycol polymers, amide polymers, or may include segments of alkylene chains, polyalkylene glycols and/or amide polymers. In certain embodiments, the linker comprises a polyethylene glycol segment having from 1 to 6 ethylene glycol units.

A variety of non-cleavable linkers used to link drugs to antibodies have been described. (See, Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255, the contents of which are incorporated herein by reference). All of these linkers may be included in the ADCs described herein.

In certain embodiments, the linker is non-cleavable in vivo, for example a linker according to structural formula (VIa), (VIb), (VIc) or (VId) (as illustrated, the linkers include a group suitable for covalently linking the linker to an antibody:

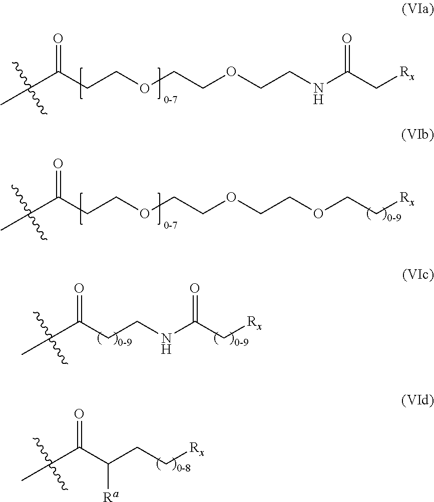

or salts thereof, wherein:
R^{a} is selected from hydrogen, alkyl, sulfonate and methyl sulfonate;
R^{x} is a moiety including a functional group capable of covalently linking the linker to an antibody; and
[symbol] represents the point of attachment of the linker to the Bcl-xL inhibitor.

Exemplary embodiments of linkers according to structural formula (VIa)-(VId) that may be included in the ADCs described herein include the linkers illustrated below (as illustrated, the linkers include a group suitable for covalently linking the linker to an antibody, and “ [symbol] ” represents the point of attachment to a Bcl-xL inhibitor):

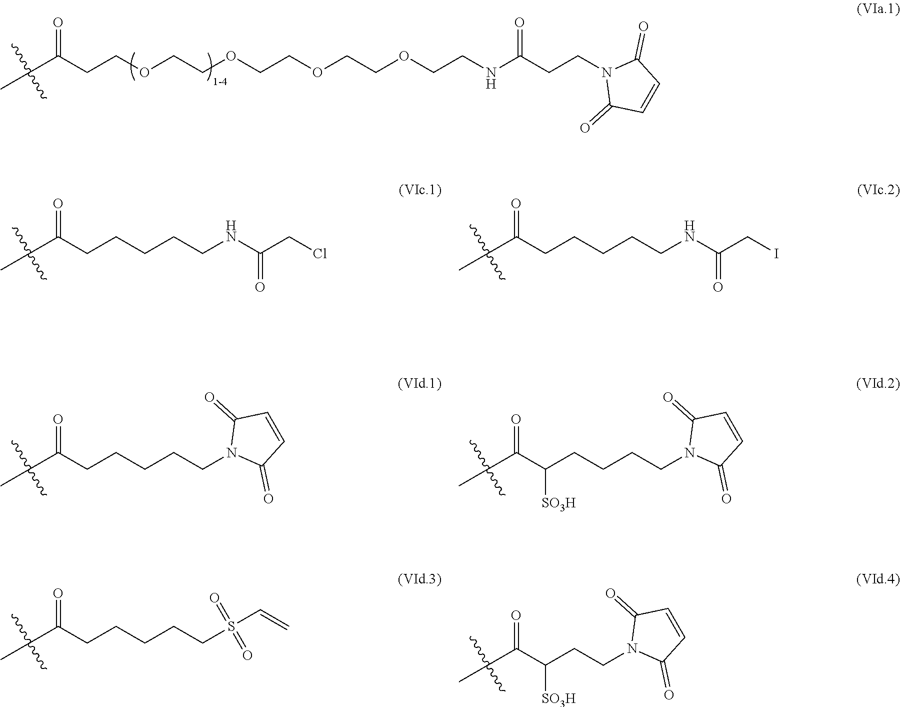

### 4.4.1.3. Groups Used to Attach Linkers to Antibodies

Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure.

One example of a “self-stabilizing” maleimide group that hydrolyzes spontaneously under antibody conjugation conditions to give an ADC species with improved stability is depicted in the schematic below. See U.S. Published Application No. 2013/0309256 and Lyon et al., 2014, Nat. Biotechnol. 32: 1059-1062. Thus, the maleimide attachment group is reacted with a sulfhydryl of an antibody to give an intermediate succinimide ring. The hydrolyzed form of the attachment group is resistant to deconjugation in the presence of plasma proteins.

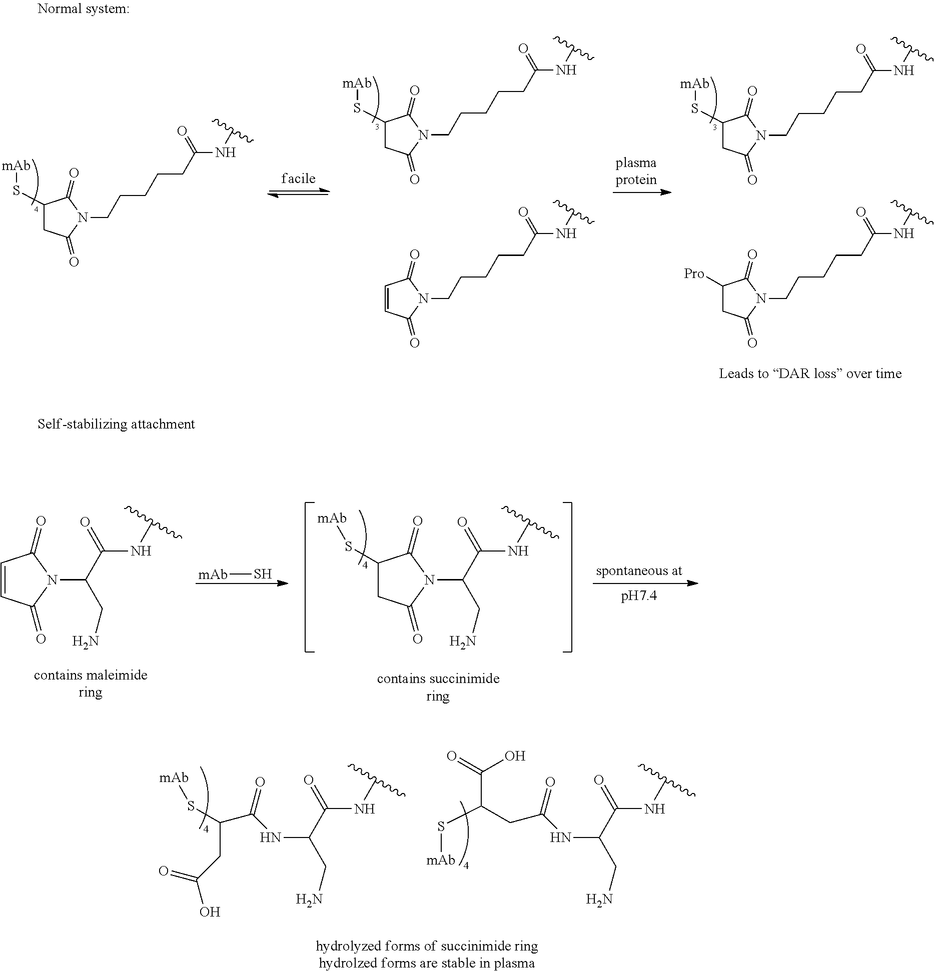

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize homogenous DAR4 ADCs by full reduction of IgGs (to give 4 pairs of sulfhydryls) followed by reaction with 4 equivalents of the alkylating agent. ADCs containing “bridged disulfides” are also claimed to have increased stability.

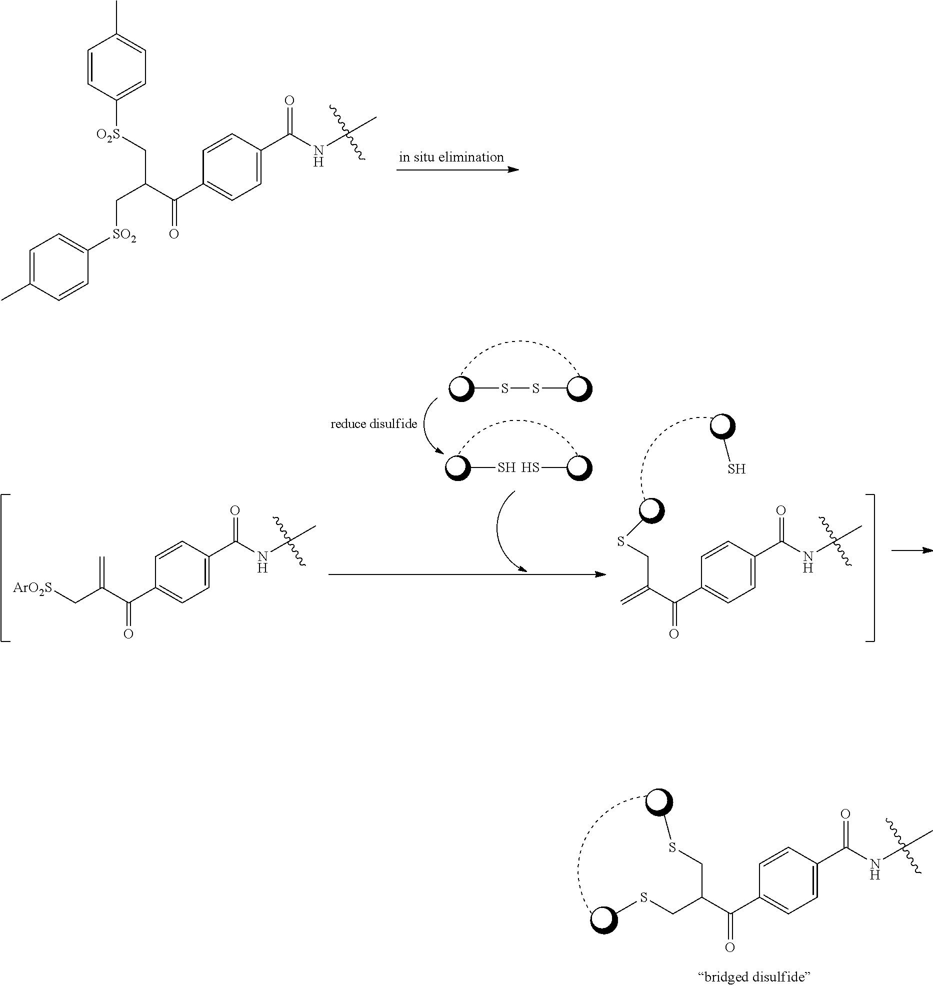

Similarly, as depicted below, a maleimide derivative that is capable of bridging a pair of sulfhydryl groups has been developed. See U.S. Published Application No. 2013/0224228.

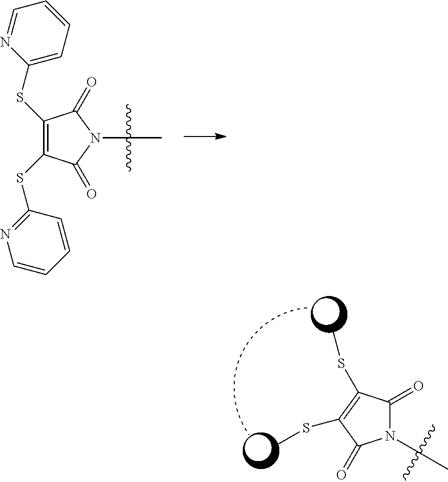 [SMILES: *N1C(=O)C(Sc2ccccn2)=C(Sc2ccccn2)C1=O ; *N1C(=O)C(SC)=C(SC)C1=O]

In certain embodiments the attachment moiety comprises the structural formulae (VIIa), (VIIb), or (VIIc):

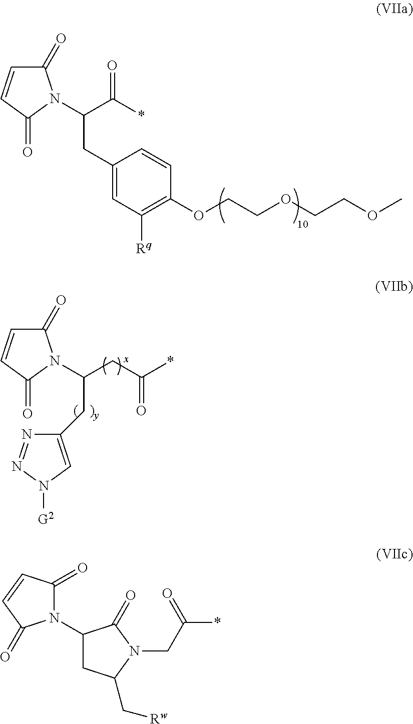

or salts thereof, wherein:
R^{q} is H or —O—(CH₂CH₂O)₁₁—CH₃;
x is 0 or 1;
y is 0 or 1;
G² is —CH₂CH₂CH₂SO₃H or —CH₂CH₂O—(CH₂CH₂O)—CH₃;
R^{w} is —O—CH₂CH₂SO₃H or —NH(CO)—CH₂CH₂O—(CH₂CH₂O)₁₂—CH; and
represents the point of attachment to the remainder of the linker.

Exemplary embodiments of linkers according to structural formula (VIIIa) and (VIIb) that may be included in the ADCs described herein include the linkers illustrated below (as illustrated, the linkers include a group suitable for covalently linking the linker to an antibody):

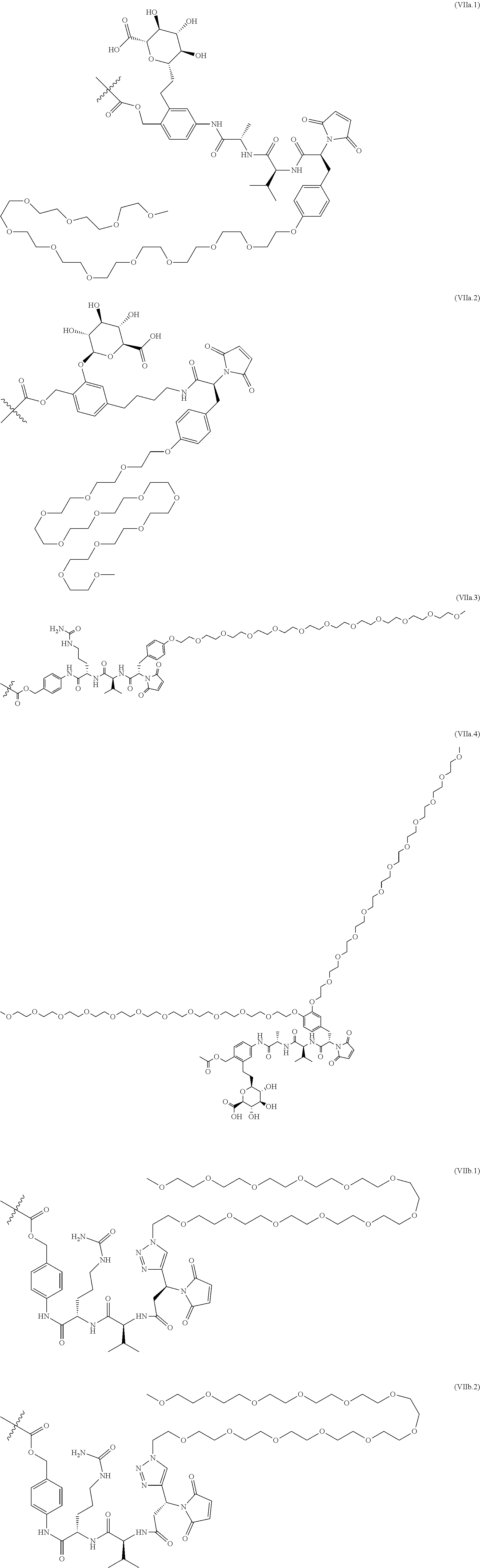

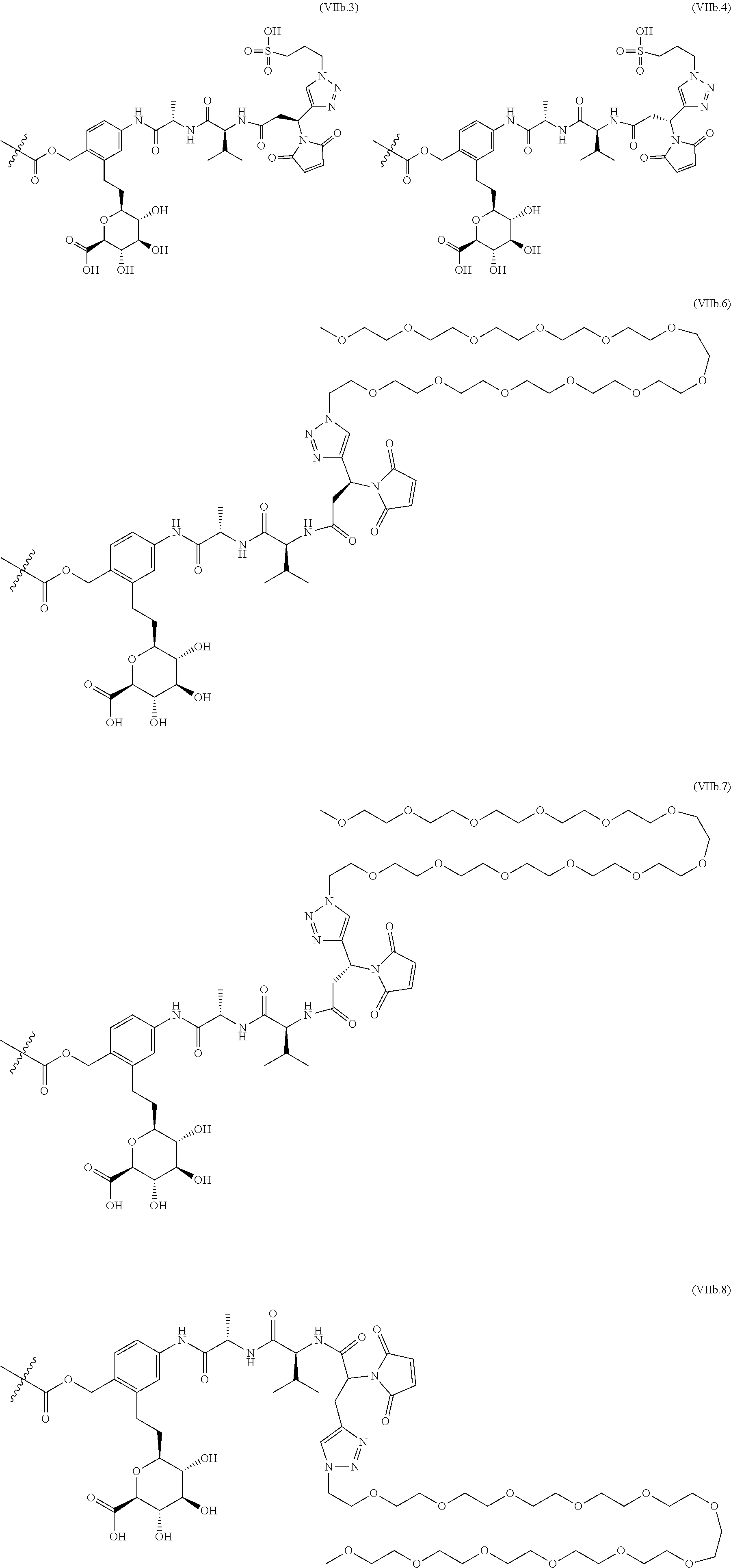

Exemplary embodiments of linkers according to structural formula (VIIc) that may be included in the ADCs described herein include the linkers illustrated below (as illustrated, the linkers include a group suitable for covalently linking the linker to an antibody):

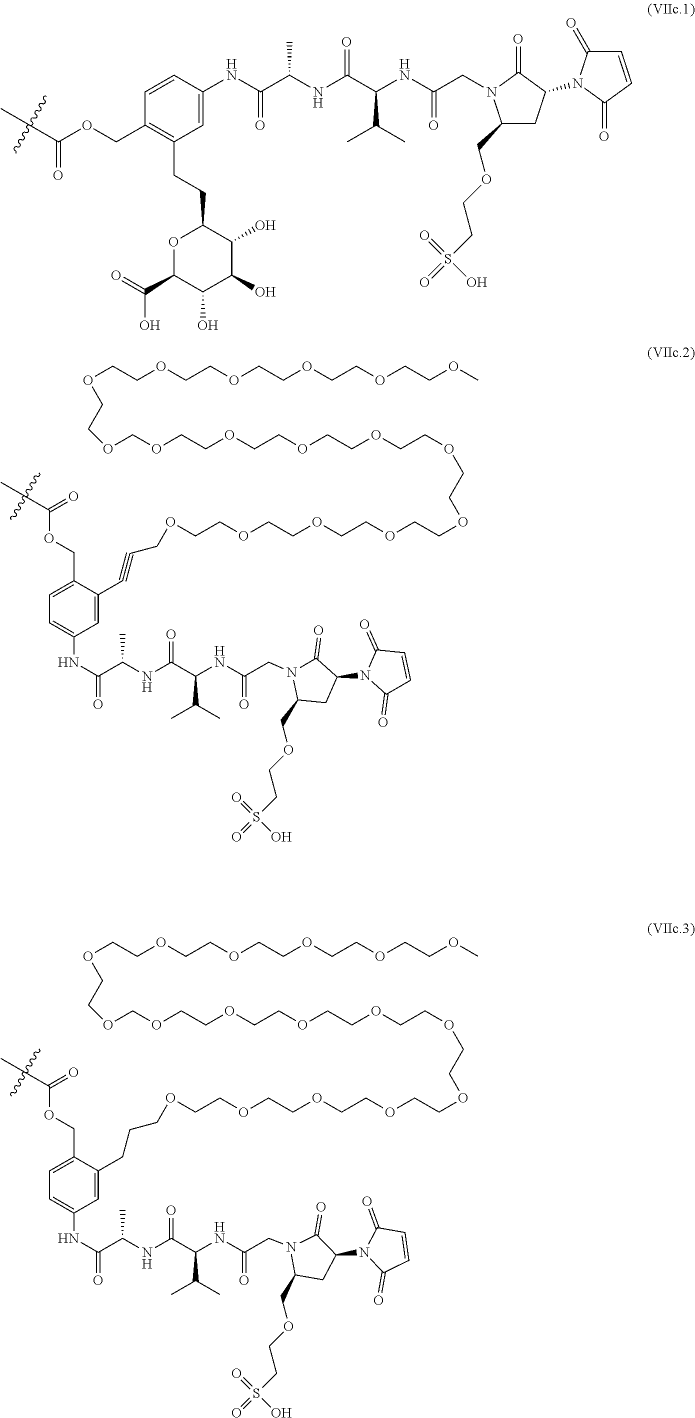 [SMILES: *C(=O)OCc1ccc(NC(=O)[C@H](C)NC(=O)[C@@H](NC(=O)CN2C(=O)[C@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1C#CCOCCOCCOCCOCCOCCOCCOCCOCCOCCOCOCCOCCOCCOCCOCCOCCOC ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CCCOCCOCCOCCOCCOCCOCCOCCOCCOCCOCOCCOCCOCCOCCOCCOCCOC]

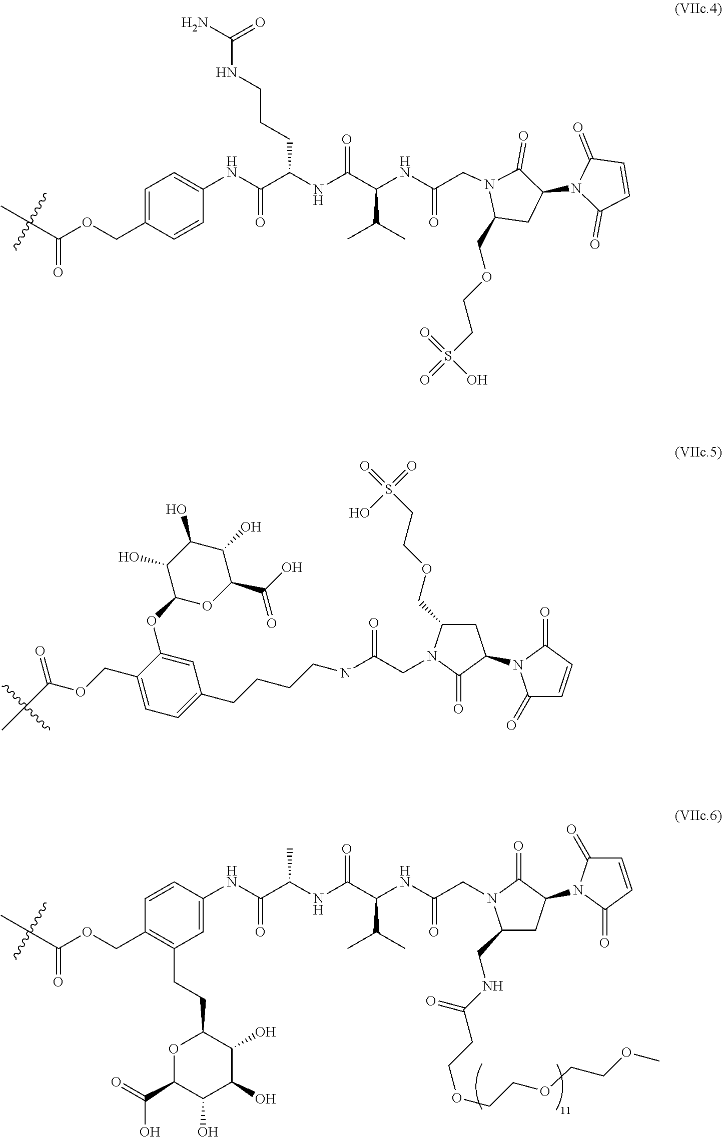

### 4.4.1.4. Linker Selection Considerations

As is known by skilled artisans, the linker selected for a particular ADC may be influenced by a variety of factors, including but not limited to, the site of attachment to the antibody (e.g., lys, cys or other amino acid residues), structural constraints of the drug pharmacophore and the lipophilicity of the drug. The specific linker selected for an ADC should seek to balance these different factors for the specific antibody/drug combination. For a review of the factors that are influenced by choice of linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the linker is selected to attenuate the bystander killing effect caused by cellular metabolites of the ADC. In certain embodiments, the linker is selected to increase the bystander killing effect.

The properties of the linker may also impact aggregation of the ADC under conditions of use and/or storage. Typically, ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the linker were hydrophobic, due to aggregation of the ADC (King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the Bcl-xL inhibitor is hydrophobic in nature, it may be desirable to select linkers that are relatively hydrophilic as a means of reducing ADC aggregation, especially in instances where DARS greater than 3-4 are desired. Thus, in certain embodiments, the linker incorporates chemical moieties that reduce aggregation of the ADCs during storage and/or use. A linker may incorporate polar or hydrophilic groups such as charged groups or groups that become charged under physiological pH to reduce the aggregation of the ADCs. For example, a linker may incorporate charged groups such as salts or groups that deprotonate, e.g., carboxylates, or protonate, e.g., amines, at physiological pH.

Exemplary polyvalent linkers that have been reported to yield DARs as high as 20 that may be used to link numerous Bcl-xL inhibitors to an antibody are described in U.S. Pat. No. 8,399,512; U.S. Published Application No. 2010/0152725; U.S. Pat. Nos. 8,524,214; 8,349,308; U.S. Published Application No. 2013/189218; U.S. Published Application No. 2014/017265; WO 2014/093379; WO 2014/093394; WO 2014/093640, the content of which are incorporated herein by reference in their entireties.

In particular embodiments, the aggregation of the ADCs during storage or use is less than about 40% as determined by size-exclusion chromatography (SEC). In particular embodiments, the aggregation of the ADCs during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### 4.5. Antibodies

The antibody of an ADC may be any antibody that binds, typically but not necessarily specifically, an antigen expressed on the surface of a target cell of interest. The antigen need not, but in some embodiments, is capable of internalizing an ADC bound thereto into the cell. Target cells of interest will generally include cells where induction of apoptosis via inhibition of anti-apoptotic Bcl-xL proteins is desirable, including, by way of example and not limitation, tumor cells that express or over-express Bcl-xL. Target antigens may be any protein, glycoprotein, polysaccharide, lipoprotein, etc. expressed on the target cell of interest, but will typically be proteins that are either uniquely expressed on the target cell and not on normal or healthy cells, or that are over-expressed on the target cell as compared to normal or healthy cells, such that the ADCs selectively target specific cells of interest, such as, for example, tumor cells. As will be appreciated by skilled artisans, the specific antigen, and hence antibody, selected will depend upon the identity of the desired target cell of interest. In specific embodiments, the antibody of the ADC is an antibody suitable for administration to humans.

Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

References to “VI” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The term “antibody” herein is used in the broadest sense and refers to an immunoglobulin molecule that specifically binds to, or is immunologically reactive with, a particular antigen, and includes polyclonal, monoclonal, genetically engineered and otherwise modified forms of antibodies, including but not limited to murine, chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′)₂, Fab, Fv, rIgG, and scFv fragments. The term “scFv” refers to a single chain Fv antibody in which the variable domains of the heavy chain and the light chain from a traditional antibody have been joined to form one chain.

Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “antibody fragment” refers to a portion of a full-length antibody, generally the target binding or variable region. Examples of antibody fragments include Fab, Fab′, F(ab′)₂ and Fv fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. “Single-chain Fv” or “scFv” antibody fragments comprise the VH and VL domains of an antibody in a single polypeptide chain. Generally, the Fv polypeptide further comprises a polypeptide linker between the VH and VL domains which enables the scFv to form the desired structure for target binding. “Single domain antibodies” are composed of a single VH or VL domains which exhibit sufficient affinity to the target. In a specific embodiment, the single domain antibody is a camelized antibody (see, e.g., Riechmann, 1999, Journal of Immunological Methods 231:25-38).

The Fab fragment contains the constant domain of the light chain and the first constant domain (CH₁) of the heavy chain. Fab′ fragments differ from Fab fragments by the addition of a few residues at the carboxyl terminus of the heavy chain CHI domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

Both the light chain and the heavy chain variable domains have complementarity determining regions (CDRs), also known as hypervariable regions. The more highly conserved portions of variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The CDRs in each chain are held together in close proximity by the FR regions and, with the CDRs from the other chain, contribute to the formation of the target binding site of antibodies (see Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al., unless otherwise indicated.

In certain embodiments, the antibodies of the ADCs in the disclosure are monoclonal antibodies. The term “monoclonal antibody” (mAb) refers to an antibody that is derived from a single copy or clone, including e.g., any eukaryotic, prokaryotic, or phage clone, and not the method by which it is produced. Preferably, a monoclonal antibody of the disclosure exists in a homogeneous or substantially homogeneous population. Monoclonal antibody includes both intact molecules, as well as, antibody fragments (such as, for example, Fab and F(ab′)₂ fragments) which are capable of specifically binding to a protein. Fab and F(ab′)₂ fragments lack the Fc fragment of intact antibody, clear more rapidly from the circulation of the animal, and may have less non-specific tissue binding than an intact antibody (Wahl et al., 1983, J. Nucl. Med. 24:316). Monoclonal antibodies useful with the present disclosure can be prepared using a wide variety of techniques known in the art including the use of hybridoma, recombinant, and phage display technologies, or a combination thereof. The antibodies of the disclosure include chimeric, primatized, humanized, or human antibodies.

While in most instances antibodies are composed of only the genetically-encoded amino acids, in some embodiments non-encoded amino acids may be incorporated at specific locations to control the number of Bcl-xL inhibitors linked to the antibody, as well as their locations. Examples of non-encoded amino acids that may be incorporated into antibodies for use in controlling stoichiometry and attachment location, as well as methods for making such modified antibodies are discussed in Tian et al., 2014, Proc Nat'l Acad Sci USA 111(5):1766-1771 and Axup et al., 2012, Proc Nat'l Acad Sci USA 109(40):16101-16106 the entire contents of which are incorporated herein by reference. In certain embodiments, the non-encoded amino acids limit the number of Bcl-xL inhibitors per antibody to about 1-8 or about 2-4.

In certain embodiments, the antibody of the ADCs described herein is a chimeric antibody. The term “chimeric” antibody as used herein refers to an antibody having variable sequences derived from a non-human immunoglobulin, such as rat or mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816397, which are incorporated herein by reference in their entireties.

In certain embodiments, the antibody of the ADCs described herein is a humanized antibody. “Humanized” forms of non-human (e.g., murine) antibodies are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See. e.g., Riechmann et al., 1988, Nature 332:323-7; U.S. Pat. Nos. 5,530,101; 5,585,089; 5,693.761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prol. Eng. 7:805-814; Roguska et al., 1994, Proc. Natl. Acad. Sci. 91:969-973; and U.S. Pat. No. 5,565,332, all of which are hereby incorporated by reference in their entireties.

In certain embodiments, the antibody of the ADCs described herein is a human antibody. Completely “human” antibodies can be desirable for therapeutic treatment of human patients. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. U.S. Pat. Nos. 4,444,887 4,716,111, 6,114,598, 6,207,418, 6,235,883, 7,227,002, 8,809,151 and U.S. Published Application No. 2013/189218, the contents of which are incorporated herein by reference in their entireties. Human antibodies can also be produced using transgenic mice which are incapable of expressing functional endogenous immunoglobulins, but which can express human immunoglobulin genes. See, e.g., U.S. Pat. Nos. 5,413,923 5,625,126, 5,633,425, 5,569,825; 5,661,016; 5,545,806; 5,814,318; 5,885,793; 5,916,771; 5,939,598; 7,723,270; 8,809,051 and U.S. Published Application No. 2013/117871, which are incorporated by reference herein in their entireties. In addition, companies such as Medarex (Princeton, N.J.), Astellas Pharma (Deerfield, Ill.), and Regeneron (Tarrytown, N.Y.) can be engaged to provide human antibodies directed against a selected antigen using technology similar to that described above. Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988. Biotechnology 12:899-903).

In certain embodiments, the antibody of the ADCs described herein is a primatized antibody. The term “primatized antibody” refers to an antibody comprising monkey variable regions and human constant regions. Methods for producing primatized antibodies are known in the art. See, e.g., U.S. Pat. Nos. 5,658,570; 5,681,722; and 5,693,780, which are incorporated herein by reference in their entireties.

In certain embodiments, the antibody of the ADCs described herein is a bispecific antibody or a dual variable domain antibody (DVD). Bispecific and DVD antibodies are monoclonal, often human or humanized, antibodies that have binding specificities for at least two different antigens. DVDs are described, for example, in U.S. Pat. No. 7,612,181, the disclosure of which is incorporated herein by reference.

In certain embodiments, the antibody of the ADCs described herein is a derivatized antibody. For example, but not by way of limitation, derivatized antibodies are typically modified by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc. Additionally, the derivative can contain one or more non-natural amino acids, e.g., using ambrx technology (see, e.g., Wolfson, 2006, Chem. Biol. 13(10):1011-2).

In certain embodiments, the antibody of the ADCs described herein has a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fe receptor (FcR). FcR binding can be reduced by mutating the immunoglobulin constant region segment of the antibody at particular regions necessary for FcR interactions (see e.g., Canfield and Morrison, 1991. J Exp. Med. 173:1483-1491; and Lund et al., 1991, J. Immunol. 147:2657-2662).

In certain embodiments, the antibody of the ADCs described herein is modified to acquire or improve at least one constant region-mediated biological effector function relative to an unmodified antibody, e.g., to enhance FcγR interactions (See, e.g., US 2006/0134709). For example, an antibody with a constant region that binds FcγRIIA, FcγRIIB and/or FcγRIIIA with greater affinity than the corresponding wild type constant region can be produced according to the methods described herein.

In certain specific embodiments, the antibody of the ADCs described herein is an antibody that binds tumor cells, such as an antibody against a cell surface receptor or a tumor-associated antigen (TAA). In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to the surface of the non-cancerous cells. Such cell surface receptor and tumor-associated antigens are known in the art, and can prepared for use in generating antibodies using methods and information which are well known in the art.

Examples of cell surface receptor and TAAs to which the antibody of the ADCs described herein may be targeted include, but are not limited to, the various receptors and TAAs listed below. For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to the listed cell surface receptors and TAAs are available in public databases such as GenBank. The sequences and disclosures of the references cited below are expressly incorporated herein by reference.

### 4.5.1 Exemplary Cell Surface Receptors and TAAs

Examples of cell surface receptor and TAAs to which the antibody of the ADCs described herein may be targeted include, but are not limited to, the various receptors and TAAs listed below. For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to the listed cell surface receptors and TAAs are available in public databases such as GenBank.

4-1BB

5AC

5T4

Alpha-fetoprotein

angiopoietin 2

ASLG659

TCL1

BMPR1B

Brevican (BCAN, BEHAB)

C242 antigen

C5

CA-125

CA-125 (imitation)

CA-IX (Carbonic anhydrase 9)

CCR4

CD140a

CD152

CD19

CD20

CD200

CD21 (C3DR) 1)

CD22 (B-cell receptor CD22-B isoform)

CD221

CD23 (gE receptor)

CD28

CD30 (TNFRSF8)

CD33

CD37

CD38(cyclic ADP ribose hydrolase)

CD4

CD40

CD44 v6

CD51

CD52

CD56

CD70

CD72 (Lyb-2, B-cell differentiation antigen CD72)

CD74

CD79a (CD79A, CD79α, immunoglobulin-associated alpha) Genbank accession No. NP_001774.10)

CD79b (CD79B, CD79β, B29)

CD80

CEA

CEA-related antigen

ch4D5

CLDN18.2

CRIPTO (CR, CR1, CRGF, TDGF1 teratocarcinoma-derived growth factor)

CTLA-4

CXCR5

DLL4

DR5

E16 (LAT1, SLC7A5) EGFL7

EGFR

EpCAM

EphB2R (DRT, ERK, Hek5, EPHT3, Tyro5)

Episialin

ERBB3

ETBR (Endothelin type B receptor)

FCRH1 (Fc receptor-like protein 1)

FcRH2 (IFGP4, IRTA4, SPAP1, SPAP1B, SPAP1C, SH2 domain containing phosphatase anchor protein

Fibronectin extra domain-B

Folate receptor 1

Frizzled receptor

GD2

GD3 ganglioside

GEDA

GPNMB

HER1

HER2 (ErbB2)

HER2/neu

HER3

HGF

HLA-DOB

HLA-DR

Human scatter factor receptor kinase

IGF-1 receptor

IgG4

IL-13

IL20Rα (IL20Rα, ZCYTOR7)

IL-6

ILGF2

ILFR1R

integrin α

integrin α₅β₁

Integrin αᵥβ₃

IRTA2 (Immunoglobulin superfamily receptor translocation associated 2, Gene Chromosome 1q21)

Lewis-Y antigen

LY64 (RP105)

MCP-1

MDP (DPEP1)

MPF (MSLN, SMR, mesothelin, megakaryocyte potentiating factor)

MS4A1

MSG783 (RNF124, hypothetical protein FLJ20315)

MUC1

Mucin CanAg

Napi3 (NAPI-3B, NPTIIb, SLC34A2, type II sodium-dependent phosphate transporter 3b)

NCA (CEACAM6)

P2X5 (Purinergic receptor P2X ligand-gated ion channel 5)

PD-1

PDCD1

PDGF-R α

Prostate specific membrane antigen

PSCA (Prostate stem cell antigen precursor)

PSCA hlg

RANKL

RON

SDC1

Sema 5b

SLAMF7 (CS-1)

STEAP1

STEAP2 (HGNC_8639, PCANAP1, STAMP1, STEAP2, STMP, prostate cancer associated gene 1)

TAG-72

TEM1

Tenascin C

TENB2, (TMEFF2, tomoregulin, TPEF, HPP1, TR)

TGF-β

TRAIL-E2

TRAIL-R1

TRAIL-R2

TrpM4 (BR22450, FLJ20041, TRPM4, TRPM4B, transient receptor potential cation channel subfamily M, member 4)

TA CTAA16.88

TWEAK-R

TYRP1 (glycoprotein 75)

VEGF

VEGF-A

EGFR-1

VEGFR-2

Vimentin

### 4.5.2 Exemplary Antibodies

Exemplary antibodies to be used with ADCs of the disclosure include but are not limited to 3F8 (GD2), Abagovomab (CA-125 (imitation)), Adecatumumab (EpCAM, Afutuzumab (CD20), Alacizumab pegol (VEGFR2), ALD518 (IL-6), Alemtuzumab (CD52), Altumomab pentetate (CEA), Amatuximab (Mesothelin), Anatumomab mafenatox (TAG-72), Apolizumab (HLA-DR), Arcitumomab (CEA). Bavituximab (Phosphatidylserine), Bectumomab (CD22), Belimumab (BAFF), Besilesomab (CEA-related antigen), Bevacizumab (VEGF-A), Bivatuzumab mertansine (CD44 v6), Blinatumomab (CD19), Brentuximab vedotin ((CD30 (TNFRSF8)), Cantuzumab mertansine (Mucin CanAg), Cantuzumab ravtansine (MUC1), Capromab pendetide (Prostatic carcinoma cells), Carlumab (MCP-1), Catumaxomab (EpCAM, CD3), CC49 (Tag-72), cBR96-DOX ADC (Lewis-Y antigen), Cetuximab (EGFR), Citatuzumab bogatox (EpCAM), Cixutumumab (IGF-1 receptor), Clivatuzumab tetraxetan (MUC1), Conatumumab (TRAIL-E2), Dacetuzumab (CD40), Dalotuzumab (Insulin-like growth factor I receptor), Daratumumab ((CD38 (cyclic ADP ribose hydrolase)). Demcizumab (DLL4), Denosumab (RANKL), Detumomab (B-lymphoma cell), Drozitumab (DR5), Dusigitumab (ILGF2), Ecromeximab (GD3 ganglioside), Eculizumab (CS), Edrecolomab (EpCAM), Elotuzumab (SLAMF7), Elsilimomab (IL-6), Enavatuzumab (TWEAK receptor), Enoticumab (DLL4), Ensituximab (5AC), Epitumomab cituxetan (Episialin), Epratuzumab (CD22), Ertumaxomab ((HER2/neu, CD3)), Etaracizumab (Integrin αᵥβ₃), Farletuzumab (Folate receptor 1), FBTA05 (CD20), Ficlatuzumab (HGF), Figitumumab (IGF-1 receptor), Flanvotumab ((TYRP1 (glycoprotein 75)), Fresolimumab (TGF-β), Galiximab (CD80), Ganitumab (IGF-I), Gemtuzumab ozogamicin (CD33), Girentuximab ((Carbonic anhydrase 9 (CA-IX)), Glembatumumab vedotin (GPNMB), Ibritumomab tiuxetan (CD20), Icrucumab (VEGFR-1), Igovomab (CA-125), IMAB362 (CLDN18.2), Imgatuzumab (EGFR), Indatuximab ravtansine (SDC1), Intetumumab (CD51), Inotuzumab ozogamicin (CD22), Ipilimumab (CD 152), Iratumumab ((CD30 (TNFRSF8)), Labetuzumab (CEA), Lambrolizumab (PDCD1), Lexatumumab (TRAIL-R2), Lintuzumab (CD33), Lorvotuzumab mertansine (CD56), Lucatumumab (CD40), Lumiliximab ((CD23 (IgE receptor)), Mapatumumab (TRAIL-R1), Margetuximab (ch4D5), Matuzumab (EGFR), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Natalizumab (integrin α₄), Necitumumab (EGFR), Nesvacumab (angiopoietin 2), Nimotuzumab (EGFR), Nivolumab (IgG4), Ocaratuzumab (CD20), Ofatumumab (CD20), Olaratumab (PDGF-R α), Onartuzumab (Human scatter factor receptor kinase), Ontuxizumab (TEM 1), Oportuzumab monato (EpCAM), Oregovomab (CA-125), Otlertuzumab (CD37), Panitumumab (EGFR), Pankomab (Tumor specific glycosylation of MUC1), Parsatuzumab (EGFL7), Patritumab (HER3), Pemtumomab (MUC1), Pertuzumab (HER2/neu), Pidilizumab (PD-1), Pinatuzumab vedotin (CD22), Pritumumab (Vimentin), Racotumomab (N-glycolylneuraminic acid), Radretumab (Fibronectin extra domain-B), Ramucirumab (VEGFR2), Rilotumumab (HGF), Rituximab (CD20), Robatumumab (IGF-1 receptor), Samalizumab (CD200), Satumomab pendetide (TAG-72), Seribantumab (ERBB3), Sibrotuzumab (FAP), SGN-CD 19A (CD 19), SGN-CD33A (CD33), Siltuximab (IL-6), Solitomab (EpCAM), Sonepcizumab (Sphingosine-1-phosphate), Tabalumb (BAFF), Tacatuzumab tetraxetan (Alpha-fetoprotein), Taplitumomab paptox (CD 19), Tenatumomab (Tenascin C), Teprotumumab (CD221), TGN1412 (CD28), Ticilimumab (CTLA-4), Tigatuzumab (TRAIL-R2), TNX-650 (IL-13), Tovetumab (CD140a), Trastuzumab (HER2/neu), TRBS07 (GD2), Tremelimumab (CTLA-4), Tucotuzumab celmoleukin (EpCAM), Ublituximab (MS4A1), Urelumab (4-1BB), Vandetanib (VEGF), Vantictumab (Frizzled receptor), Volociximab (integrin α₅β₁), Vorsetuzumab mafodotin (CD70), Votumumab (Tumor antigen CTAA16.88), Zalutumumab (EGFR), Zanolimumab (CD4), Zatuximab (HER1).

In certain embodiments, the antibody of the ADC binds EGFR NCAM1 or EpCAM. In certain embodiments, the antibody of the ADC binds EGFR, EpCAM, or NCAM1. In certain embodiments, the antibody of the ADC binds EGFR or NCAM1. In certain embodiments, the antibody is selected from the group consisting of the EpCAM antibody referred to ING-1, the NCAM-1 antibody referred to as N901, and the EGFR antibody referred to as AB033.

### 4.6. Methods of Making Antibodies

The antibody of an ADC can be prepared by recombinant expression of immunoglobulin light and heavy chain genes in a host cell. For example, to express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies arc used to obtain antibody heavy and light chain genes, incorporate these genes into recombinant expression vectors and introduce the vectors into host cells, such as those described in Molecular Cloning: A Laboratory Manual. Second Edition (Sambrook, Fritsch and Maniatis (eds), Cold Spring Harbor, N.Y., 1989). Current Protocols in Molecular Biology (Ausubel, F. M. et al., eds., Greene Publishing Associates, 1989) and in U.S. Pat. No. 4,816,397.

In one embodiment, the Fc variant antibodies are similar to their wild-type equivalents but for changes in their Fc domains. To generate nucleic acids encoding such Fc variant antibodies, a DNA fragment encoding the Fc domain or a portion of the Fc domain of the wild-type antibody (referred to as the “wild-type Fc domain”) can be synthesized and used as a template for mutagenesis to generate an antibody as described herein using routine mutagenesis techniques; alternatively, a DNA fragment encoding the antibody can be directly synthesized.

Once DNA fragments encoding wild-type Fc domains are obtained, these DNA fragments can be further manipulated by standard recombinant DNA techniques, for example, to convert the constant region genes to full-length antibody chain genes. In these manipulations, a CH-encoding DNA fragment is operatively linked to another DNA fragment encoding another protein, such as an antibody variable region or a flexible linker. The term “operatively linked,” as used in this context, is intended to mean that the two DNA fragments are joined such that the amino acid sequences encoded by the two DNA fragments remain in-frame.

To express the Fc variant antibodies, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. A variant antibody light chain gene and the antibody heavy chain gene can be inserted into separate vectors or, more typically, both genes are inserted into the same expression vector.

The antibody genes are inserted into the expression vector by standard methods (e.g., ligation of complementary restriction sites on the antibody gene fragment and vector, or blunt end ligation if no restriction sites are present). Prior to insertion of the variant Fc domain sequences, the expression vector can already carry antibody variable region sequences. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, Calif., 1990). It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al., and U.S. Pat. No. 4,968,615 by Schaffner et al.

In addition to the antibody chain genes and regulatory sequences, the recombinant expression vectors can carry additional sequences, such as sequences that regulate replication of the vector in host cells (e.g., origins of replication) and selectable marker genes. The selectable marker gene facilitates selection of host cells into which the vector has been introduced (See, e.g., U.S. Pat. Nos. 4,399,216, 4,634,665 and 5,179,017, all by Axel et al.). For example, typically the selectable marker gene confers resistance to drugs, such as G418, puromycin, blasticidin, hygromycin or methotrexate, on a host cell into which the vector has been introduced. Suitable selectable marker genes include the dihydrofolate reductase (DHFR) gene (for use in DHFR⁻ host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” arc intended to encompass a wide variety of techniques commonly used for the introduction of exogenous DNA into a prokaryotic or eukaryotic host cell, e.g., electroporation, lipofection, calcium-phosphate precipitation, DEAE-dextran transfection and the like.

It is possible to express the antibodies in either prokaryotic or eukaryotic host cells. In certain embodiments, expression of antibodies is performed in eukaryotic cells, e.g., mammalian host cells, for optimal secretion of a properly folded and immunologically active antibody. Exemplary mammalian host cells for expressing the recombinant antibodies include Chinese Hamster Ovary (CHO cells) (including DHFR CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601-621), NS0 myeloma cells, COS cells, 293 cells and SP2/0 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Host cells can also be used to produce portions of intact antibodies, such as Fab fragments or scFv molecules.

In some embodiments, the antibody of an ADC can be a bifunctional antibody. Such antibodies, in which one heavy and one light chain are specific for one antigen and the other heavy and light chain are specific for a second antigen, can be produced by crosslinking an antibody to a second antibody by standard chemical crosslinking methods. Bifunctional antibodies can also be made by expressing a nucleic acid engineered to encode a bifunctional antibody.

In certain embodiments, dual specific antibodies, i.e. antibodies that bind one antigen and a second, unrelated antigen using the same binding site, can be produced by mutating amino acid residues in the light chain and/or heavy chain CDRs. Exemplary second antigens include a proinflammatory cytokine (such as, for example, lymphotoxin, interferon-γ, or interleukin-1). Dual specific antibodies can be produced, e.g., by mutating amino acid residues in the periphery of the antigen binding site (See. e.g., Bostrom et al., 2009, Science 323:1610-1614). Dual functional antibodies can be made by expressing a nucleic acid engineered to encode a dual specific antibody.

Antibodies can also be produced by chemical synthesis (e.g., by the methods described in Solid Phase Peptide Synthesis, 2^{nd} ed., 1984 The Pierce Chemical Co., Rockford, Ill.). Antibodies can also be generated using a cell-free platform (see, e.g., Chu et al., Biochemia No. 2, 2001 (Roche Molecular Biologicals)).

Methods for recombinant expression of Fc fusion proteins are described in Flanagan et al., Methods in Molecular Biology, vol. 378: Monoclonal Antibodies: Methods and Protocols.

Once an antibody has been produced by recombinant expression, it can be purified by any method known in the art for purification of an immunoglobulin molecule, for example, by chromatography (e.g., ion exchange, affinity, particularly by affinity for antigen after Protein A or Protein G selection, and sizing column chromatography), centrifugation, differential solubility, or by any other standard technique for the purification of proteins.

Once isolated, an antibody can, if desired, be further purified, e.g., by high performance liquid chromatography (See, e.g., Fisher, Laboratory Techniques In Biochemistry And Molecular Biology (Work and Burdon, eds., Elsevier, 1980)), or by gel filtration chromatography on a Superdex™ 75 column (Pharmacia Biotech AB, Uppsala, Sweden).

### 4.7. Antibody-Drug Conjugate Synthons

Antibody-Drug Conjugate synthons are synthetic intermediates used to form ADCs. The synthons are generally compounds according to structural formula (III):

D-L-R^{x} (III)

or salts thereof, wherein D is a Bcl-xL inhibitor as previously described, L is a linker as previously described, and R^{x} is a reactive group suitable for linking the synthon to an antibody. In specific embodiments, the ADC synthons are compounds according to structural formulae (IIIa) and (IIIb), or salts thereof, where the various substituents are as previously defined for structural formulae (IIa) and (IIb), respectively, and L and R are as defined for structural formula (III):

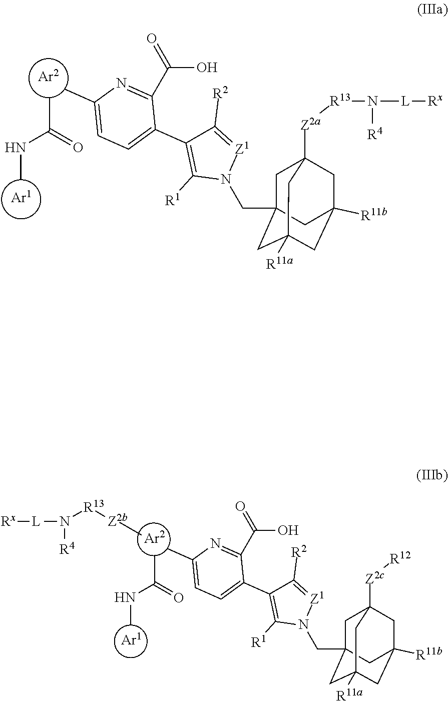

To synthesize an ADC, an intermediate synthon according to structural formula (III), or a salt thereof, is contacted with an antibody of interest under conditions in which functional group R reacts with a “complementary” functional group on the antibody, F^{x}, to form a covalent linkage.

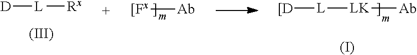

The identities of groups R^{x} and F^{x} will depend upon the chemistry used to link the synthon to the antibody. Generally, the chemistry used should not alter the integrity of the antibody, for example its ability to bind its target. Preferably, the binding properties of the conjugated antibody will closely resemble those of the unconjugated antibody. A variety of chemistries and techniques for conjugating molecules to biological molecules such as antibodies are known in the art and in particular to antibodies, are well-known. See, e.g., Amon et al., “Monoclonal Antibodies For Immunotargeting Of Drugs In Cancer Therapy,” in: Monoclonal Antibodies And Cancer Therapy, Reisfeld et al., Eds., Alan R. Liss, Inc., 1985; Hellstrom et al., “Antibodies For Drug Delivery,” in: Controlled Drug Delivery, Robinson et al., Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to an antibody.

In one embodiment, R^{x} comprises a functional group capable of linking the synthon to an amino group on an antibody. In another embodiment, R^{x} comprises an NHS-ester or an isothiocyanate. In another embodiment, R^{x} comprises a functional group capable of linking the synthon to a sulfhydryl group on an antibody. In another embodiment, R^{x} comprises a haloacetyl or a maleimide. In another embodiment, L is selected from IVa or IVb and salts thereof; and R^{x} comprises a functional group selected from the group consisting of NHS-ester, isothiocyanate, haloacetyl and maleimide.

Typically, the synthons arc linked to the side chains of amino acid residues of the antibody, including, for example, the primary amino group of accessible lysine residues or the sulfhydryl group of accessible cysteine residues. Free sulfhydryl groups may be obtained by reducing interchain disulfide bonds.

In one embodiment, LK is a linkage formed with an amino group on antibody Ab. In another embodiment, LK is an amide or a thiourea. In another embodiment, LK is a linkage formed with a sulfhydryl group on antibody Ab. In another embodiment, LK is a thioether.

In one embodiment, LK is selected from the group consisting of amide, thiourea and thioether; and m is an integer ranging from 1 to 8.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the antibody. Functional groups R suitable for covalently linking the synthons to these “converted” functional groups are then included in the synthons.

The antibody may also be engineered to include amino acid residues for conjugation. An approach for engineering antibodies to include non-genetically encoded amino acid residues useful for conjugating drugs in the context of ADCs is described in Axup et al., 2003, Proc Natl Acad Sci 109:16101-16106 and Tian et al., 2014, Proc Natl Acad Sci 111:1776-1771, as are chemistries and functional group useful for linking synthons to the non-encoded amino acids.

Exemplary synthons that may be used to make ADCs include, but are not limited to, the following synthons:

| Appln | Syn- | |
|---|---|---|
| Ex. No. | thon | Synthon Structure |
| 2.1 | BS | 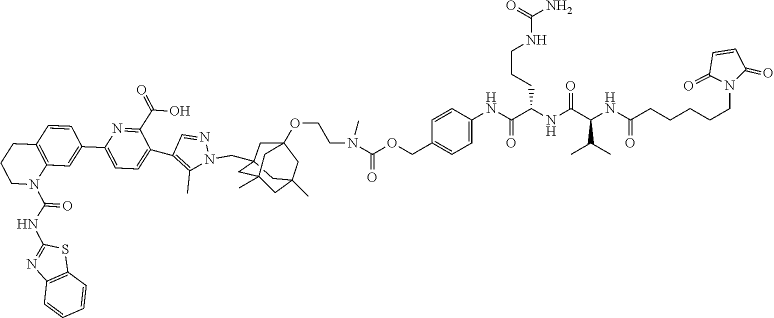 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.2 | DK | 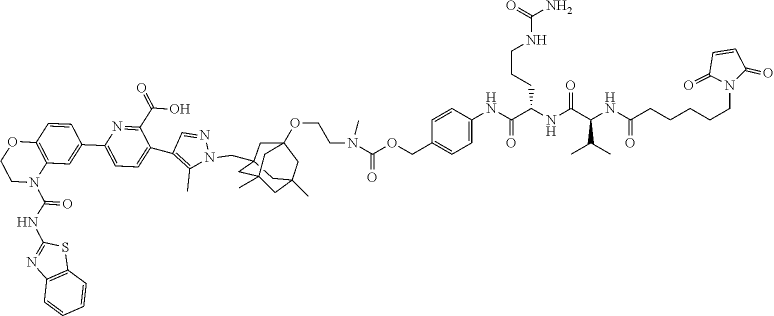 [SMILES: Cc1c(-c2ccc(-c3ccc4c(c3)N(C(=O)Nc3nc5ccccc5s3)CCO4)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.3 | DQ | 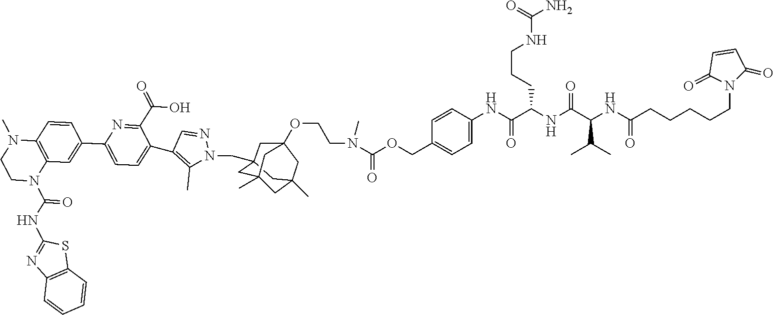 [SMILES: Cc1c(-c2ccc(-c3ccc4c(c3)N(C(=O)Nc3nc5ccccc5s3)CCN4C)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.4 | DJ | 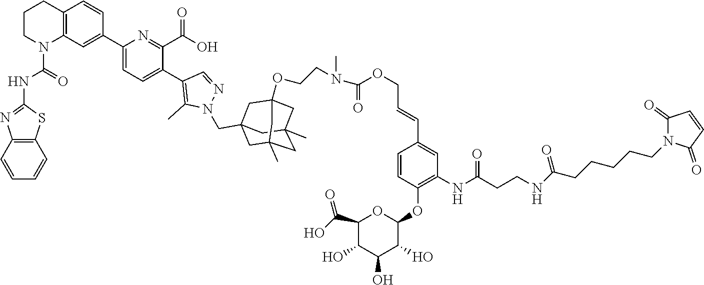 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.5 | DO | 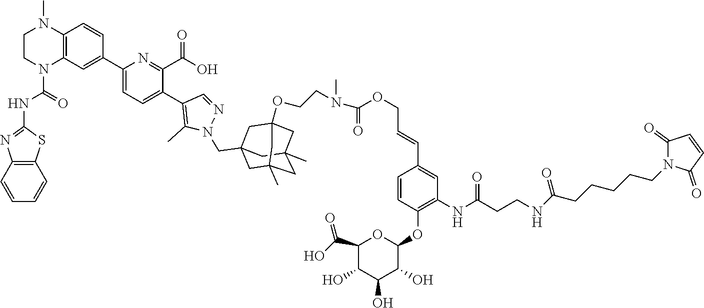 [SMILES: Cc1c(-c2ccc(-c3ccc4c(c3)N(C(=O)Nc3nc5ccccc5s3)CCN4C)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.6 | DP | 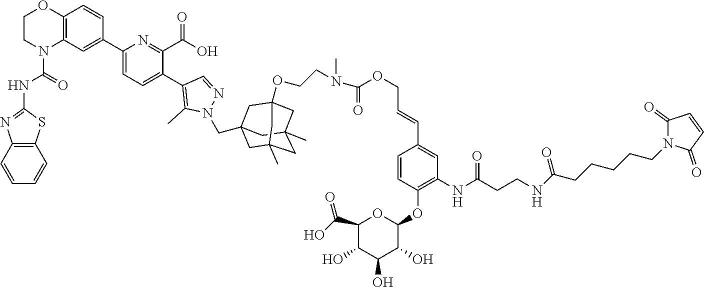 [SMILES: Cc1c(-c2ccc(-c3ccc4c(c3)N(C(=O)Nc3nc5ccccc5s3)CCO4)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.7 | HO | 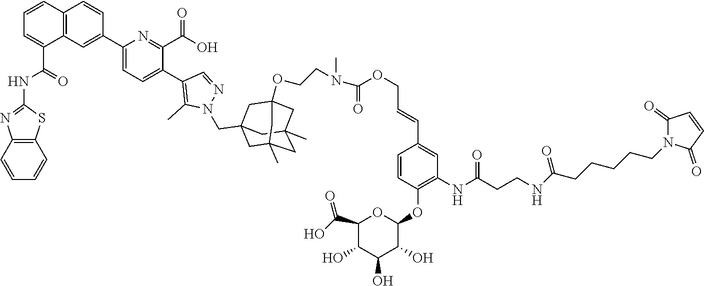 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.8 | IT | 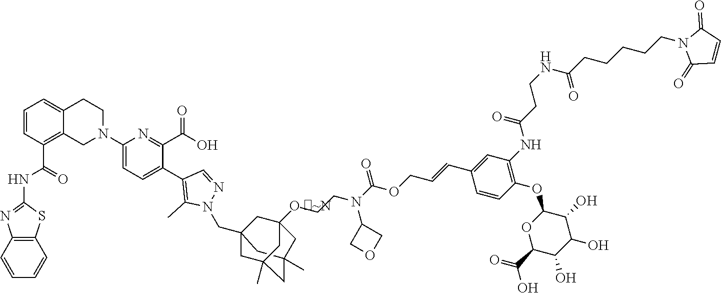 |
| 2.9 | KA | 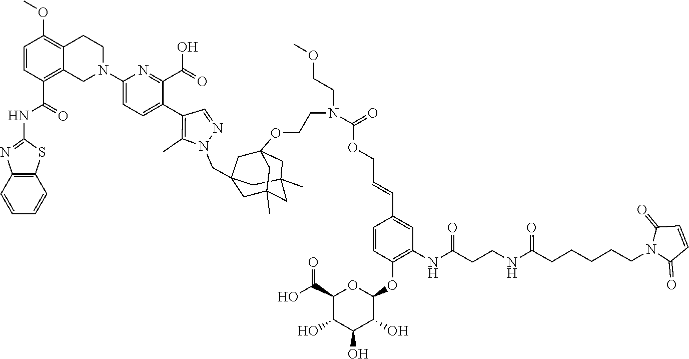 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCCCCN2C(=O)C=CC2=O)c1] |
| 2.10 | KB | 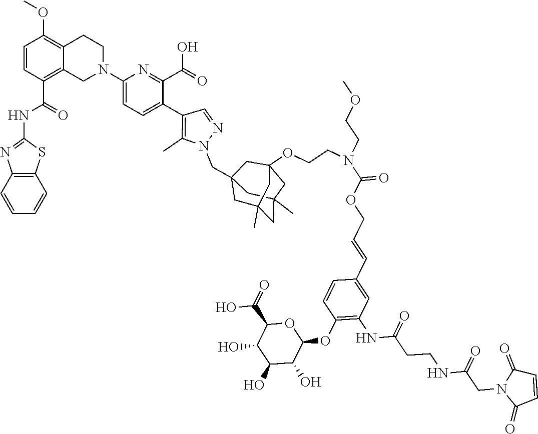 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CN2C(=O)C=CC2=O)c1] |
| 2.11 | KT | 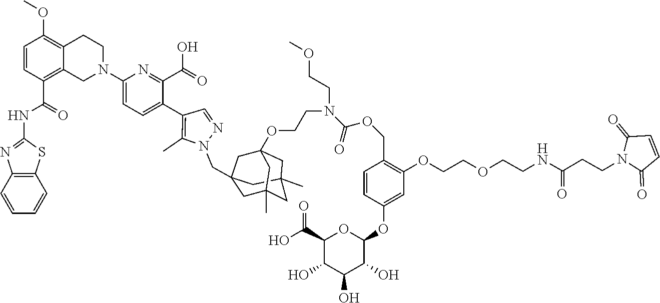 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CCN1C(=O)C=CC1=O] |
| 2.12 | KU | 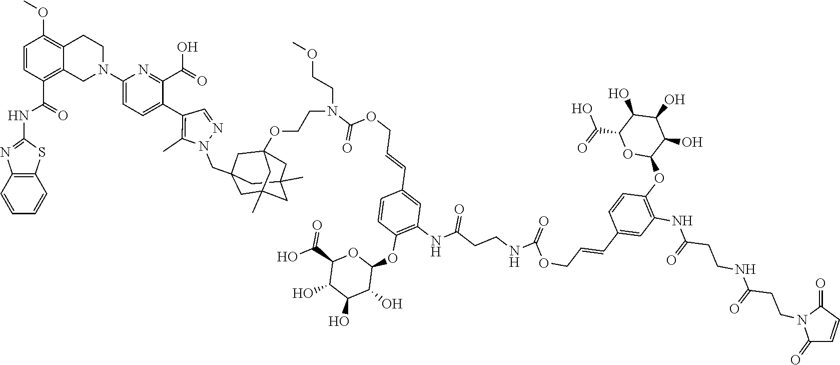 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)OC/C=C/c2ccc(O[C@H]3O[C@H](C(=O)O)[C@@H](O)[C@@H](O)[C@H]3O)c(NC(=O)CCNC(=O)CCN3C(=O)C=CC3=O)c2)c1] |
| 2.13 | KV | 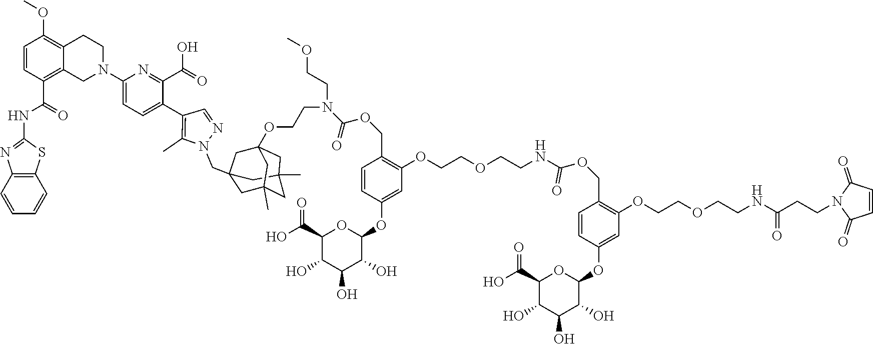 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)OCc1ccc(O[C@@H]2O[C@H](C(=O)O)[C@@H](O)[C@H](O)[C@H]2O)cc1OCCOCCNC(=O)CCN1C(=O)C=CC1=O] |
| 2.14 | KW | 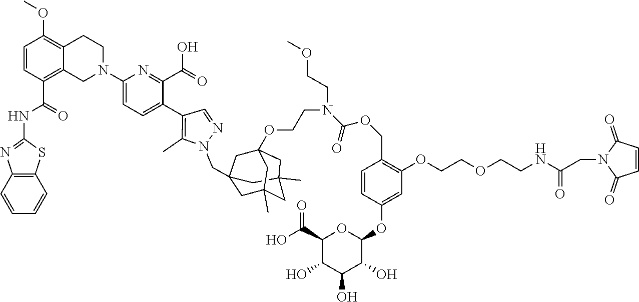 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CN1C(=O)C=CC1=O] |
| 2.15 | DC | 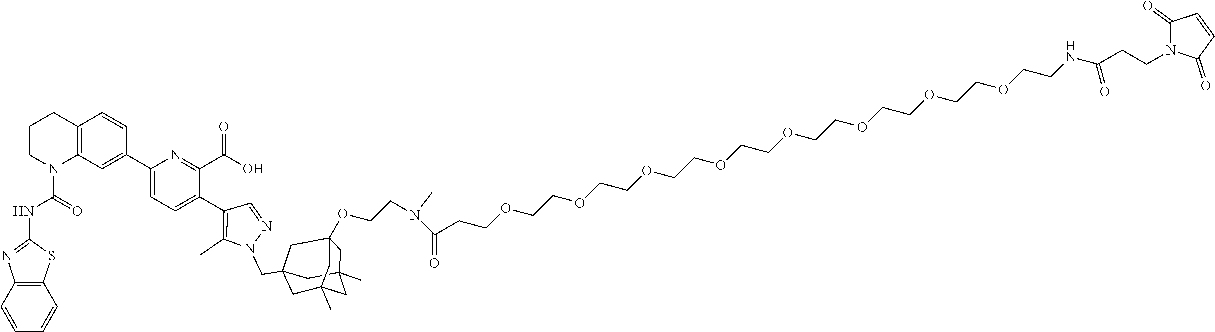 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)CCOCCOCCOCCOCCOCCOCCOCCOCCNC(=O)CCN1C(=O)C=CC1=O)(C3)C2] |
| 2.16 | KZ | 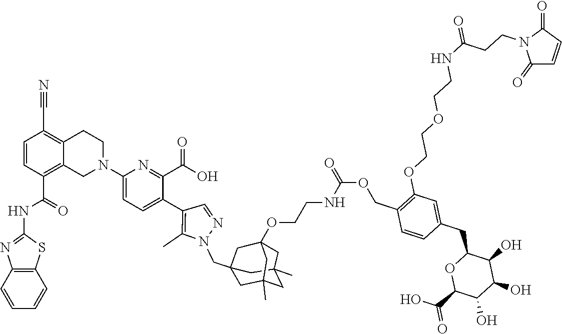 [SMILES: Cc1c(-c2ccc(N3CCc4c(C#N)ccc(C(=O)Nc5nc6ccccc6s5)c4C3)nc2C(=O)O)cnn1CC12CC3(C)CC(C)(C1)CC(OCCNC(=O)OCc1ccc(C[C@@H]4O[C@H](C(=O)O)[C@@H](O)[C@H](O)[C@@H]4O)cc1OCCOCCNC(=O)CCN1C(=O)C=CC1=O)(C3)C2] |
| 2.17 | LW | 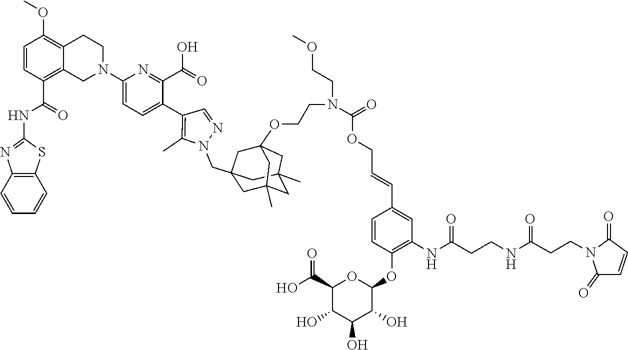 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCN2C(=O)C=CC2=O)c1] |
| 2.18 | LY | 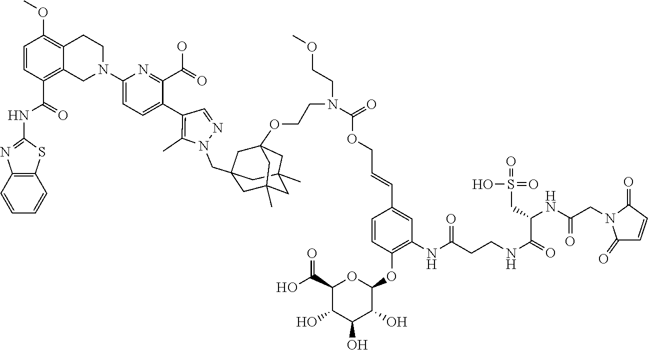 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1] |
| 2.19 | LZ | 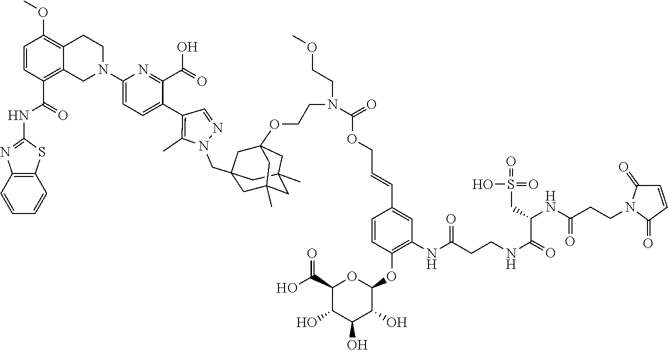 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1] |
| 2.20 | MB | 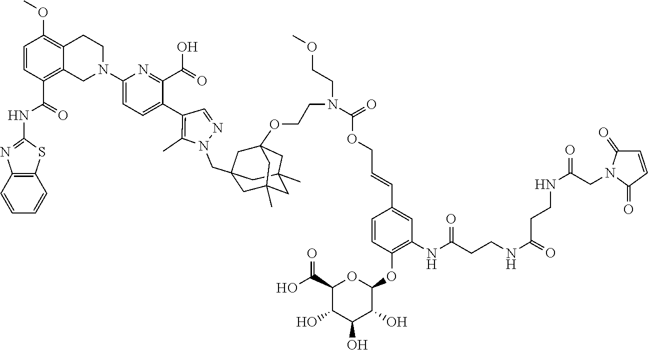 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCNC(=O)CN2C(=O)C=CC2=O)c1] |
| 2.21 | MC | 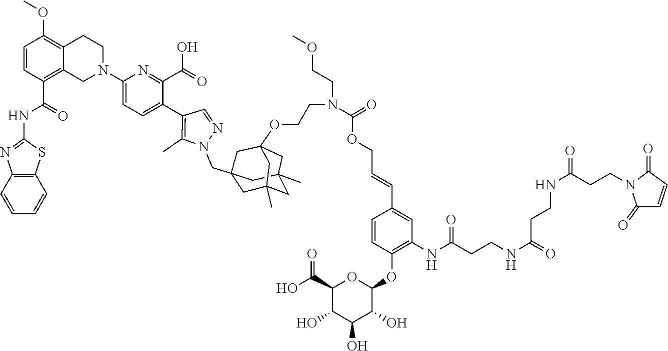 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCNC(=O)CCN2C(=O)C=CC2=O)c1] |
| 2.22 | ME | 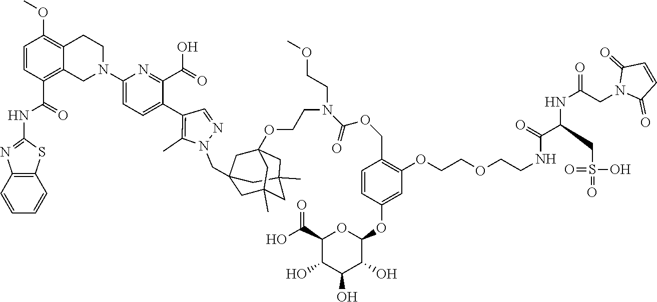 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CN1C(=O)C=CC1=O] |
| 2.23 | MF | 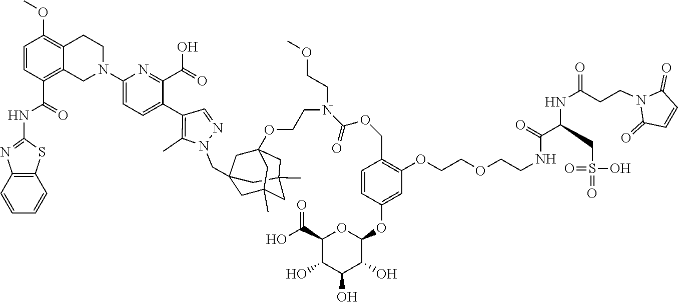 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCN1C(=O)C=CC1=O] |
| 2.24 | MH | 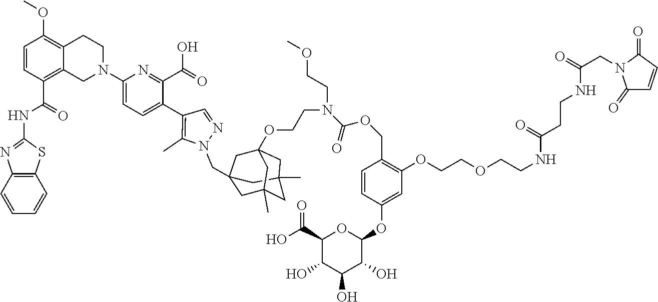 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CCNC(=O)CN1C(=O)C=CC1=O] |
| 2.25 | MI | 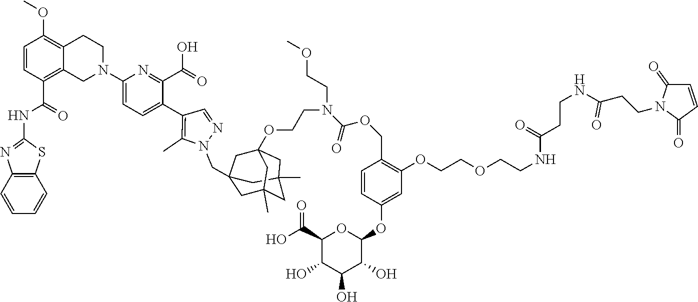 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CCNC(=O)CCN1C(=O)C=CC1=O] |
| 2.26 | NJ | 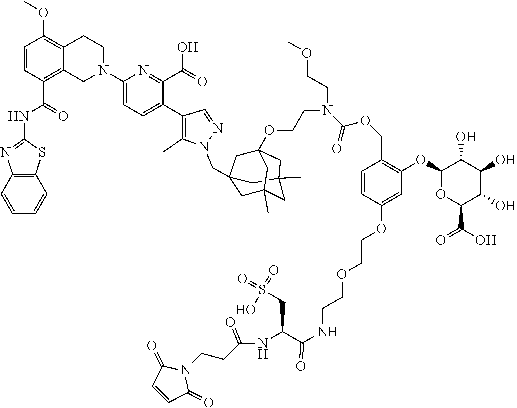 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(OCCOCCNC(=O)[C@H](CS(=O)(=O)O)NC(=O)CCN2C(=O)C=CC2=O)cc1O[C@@H]1O[C@H](C(=O)O)[C@@H](O)[C@H](O)[C@H]1O] |
| 2.27 | NK | 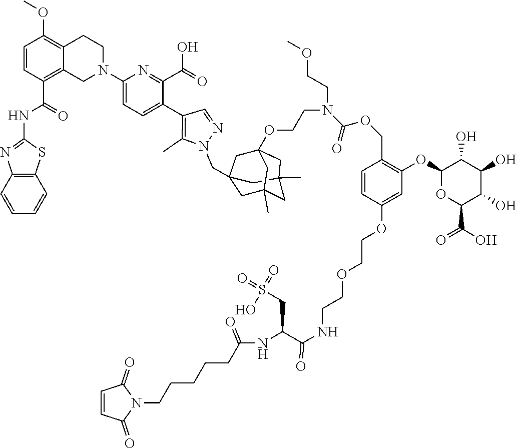 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O] |
| 2.27 | NL | 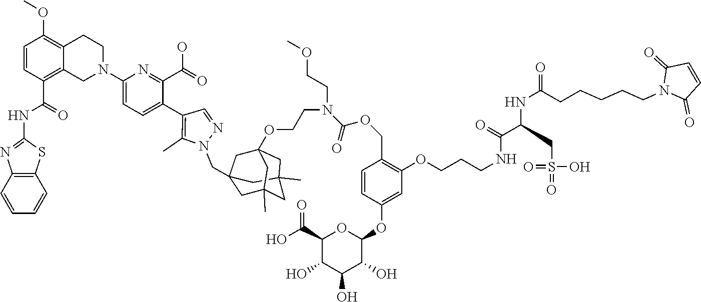 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O] |
| 2.29 | NM | 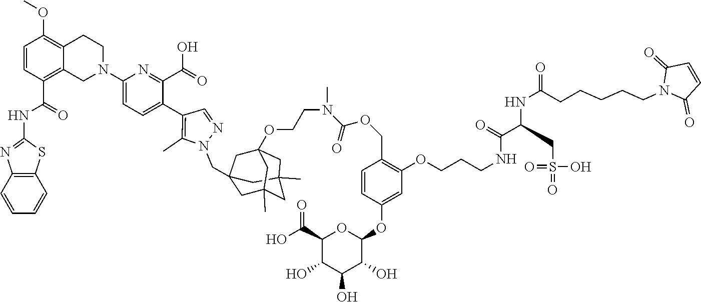 [SMILES: COc1ccc(C(=O)Nc2nc3ccccc3s2)c2c1CCN(c1ccc(-c3cnn(CC45CC6(C)CC(C)(C4)CC(OCCN(C)C(=O)OCc4ccc(O[C@@H]7O[C@H](C(=O)O)[C@@H](O)[C@H](O)[C@H]7O)cc4OCCCNC(=O)[C@H](CS(=O)(=O)O)NC(=O)CCCCCN4C(=O)C=CC4=O)(C6)C5)c3C)c(C(=O)O)n1)C2] |
| 2.30 | NR | 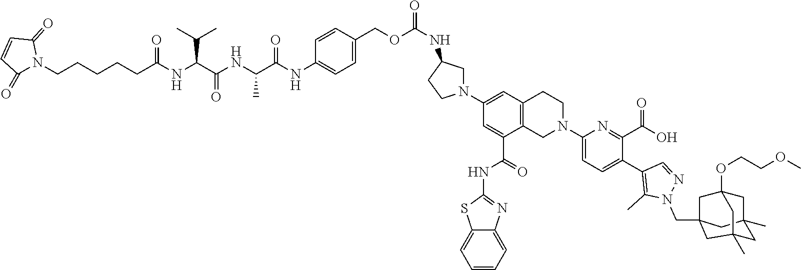 [SMILES: COCCOC12CC3(C)CC(C)(CC(Cn4ncc(-c5ccc(N6CCc7cc(N8CC[C@@H](NC(=O)OCc9ccc(NC(=O)[C@H](C)NC(=O)[C@@H](NC(=O)CCCCCN%10C(=O)C=CC%10=O)C(C)C)cc9)C8)cc(C(=O)Nc8nc9ccccc9s8)c7C6)nc5C(=O)O)c4C)(C3)C1)C2] |
| 2.31 | EB | 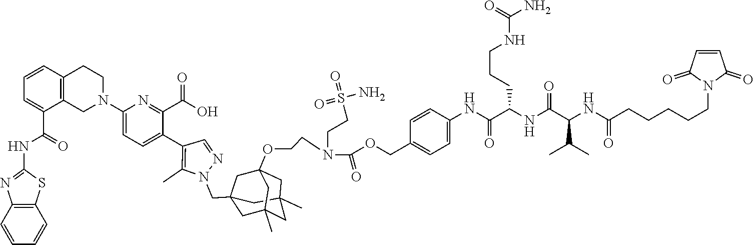 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(N)(=O)=O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.34 | OG | 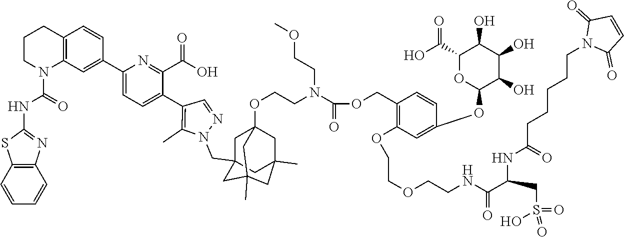 [SMILES: COCCN(CCOC12CC3(C)CC(C)(CC(Cn4ncc(-c5ccc(-c6ccc7c(c6)N(C(=O)Nc6nc8ccccc8s6)CCC7)nc5C(=O)O)c4C)(C3)C1)C2)C(=O)OCc1ccc(O[C@H]2O[C@H](C(=O)O)[C@@H](O)[C@@H](O)[C@H]2O)cc1OCCOCCNC(=O)[C@H](CS(=O)(=O)O)NC(=O)CCCCCN1C(=O)C=CC1=O] |
| 2.35 | OH | 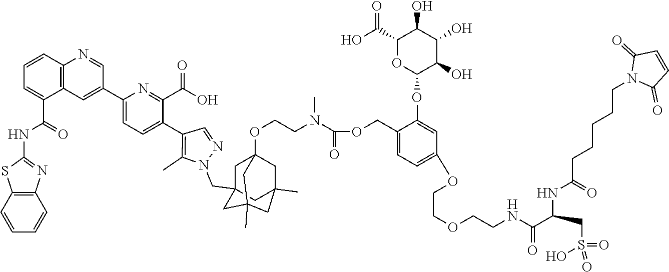 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)OCc1ccc(OCCOCCNC(=O)[C@H](CS(=O)(=O)O)NC(=O)CCCCCN4C(=O)C=CC4=O)cc1O[C@@H]1O[C@H](C(=O)O)[C@@H](O)[C@H](O)[C@H]1O)(C3)C2] |
| 2.36 | ON | 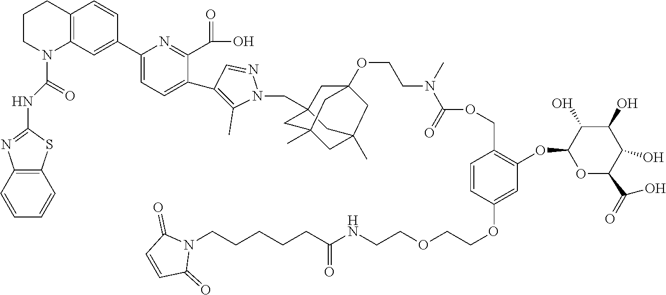 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(OCCOCCNC(=O)CCCCCN4C(=O)C=CC4=O)cc1O[C@@H]1O[C@H](C(=O)O)[C@@H](O)[C@H](O)[C@H]1O)(C3)C2] |
| 2.37 | OT | 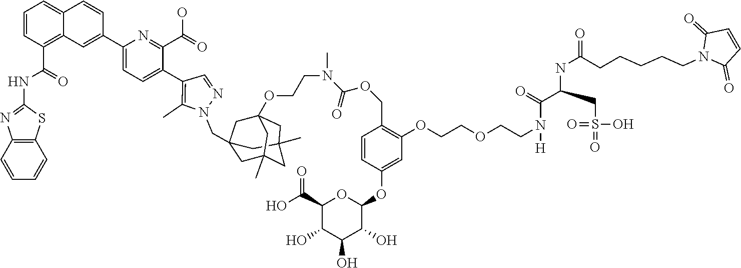 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)[C@H](CS(=O)(=O)O)NC(=O)CCCCCN1C(=O)C=CC1=O)(C3)C2] |
| 2.38 | OP | 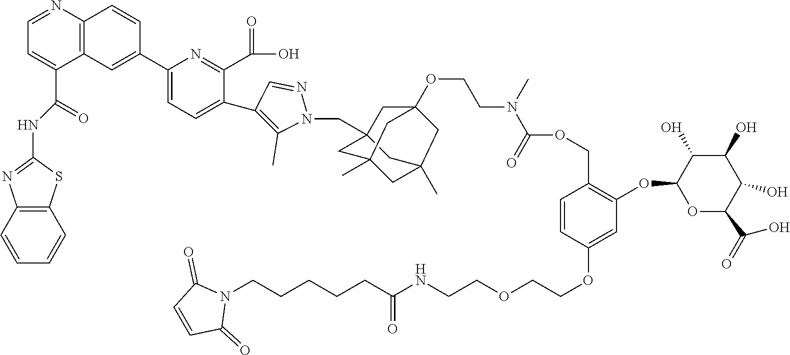 [SMILES: Cc1c(-c2ccc(-c3ccc4nccc(C(=O)Nc5nc6ccccc6s5)c4c3)nc2C(=O)O)cnn1CC12CC3(C)CC(C)(C1)CC(OCCN(C)C(=O)OCc1ccc(OCCOCCNC(=O)CCCCCN4C(=O)C=CC4=O)cc1O[C@@H]1O[C@H](C(=O)O)[C@@H](O)[C@H](O)[C@H]1O)(C3)C2] |
| 2.39 | OU | 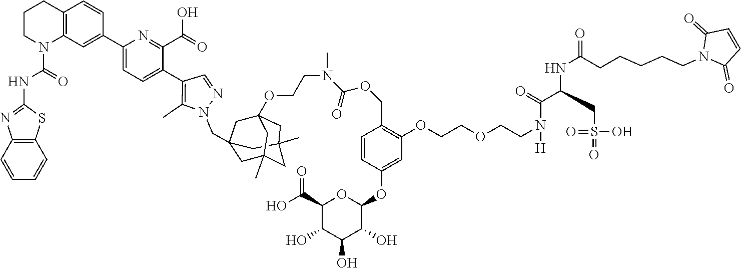 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)[C@H](CS(=O)(=O)O)NC(=O)CCCCCN1C(=O)C=CC1=O)(C3)C2] |
| 2.40 | OO | 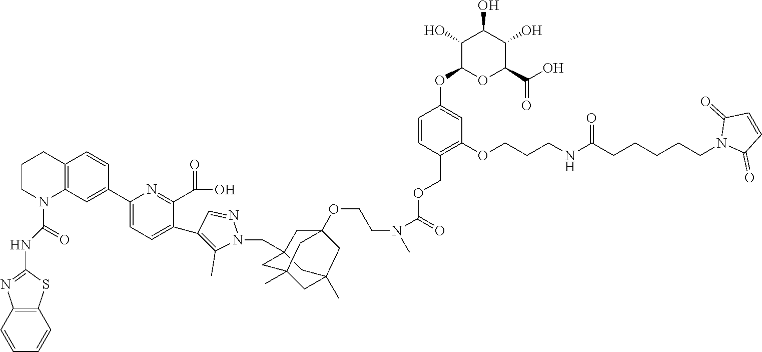 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCCNC(=O)CCCCCN1C(=O)C=CC1=O)(C3)C2] |
| 2.41 | OQ | 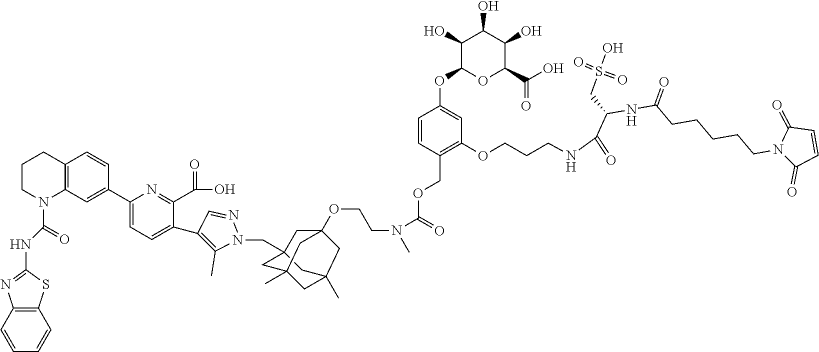 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(O[C@@H]4O[C@H](C(=O)O)[C@H](O)[C@H](O)[C@@H]4O)cc1OCCCNC(=O)[C@H](CS(=O)(=O)O)NC(=O)CCCCCN1C(=O)C=CC1=O)(C3)C2] |
| 2.42 | OR | 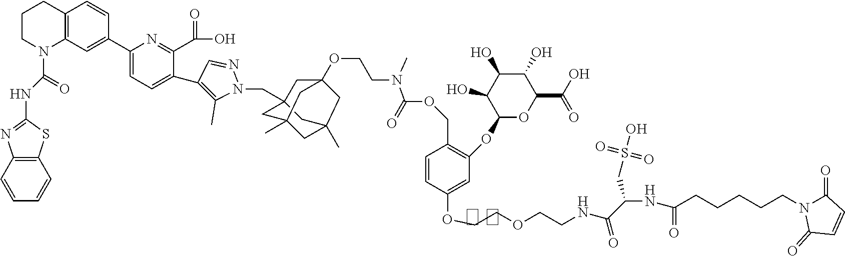 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(OCCOCCNC(=O)[C@H](CS(=O)(=O)O)NC(=O)CCCCCN4C(=O)C=CC4=O)cc1O[C@@H]1O[C@H](C(=O)O)[C@@H](O)[C@H](O)[C@@H]1O)(C3)C2] |
| 2.43 | OS | 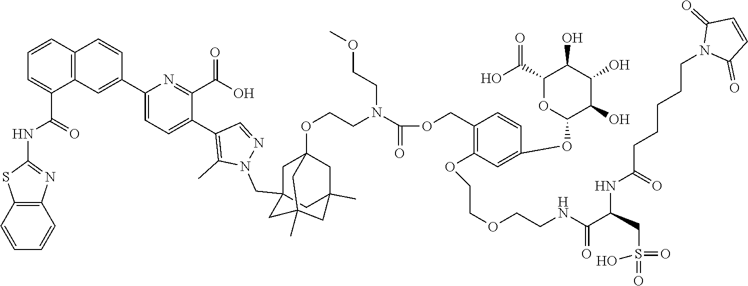 [SMILES: COCCN(CCOC12CC3(C)CC(C)(CC(Cn4ncc(-c5ccc(-c6ccc7cccc(C(=O)Nc8nc9ccccc9s8)c7c6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O] |
| 2.44 | OX | 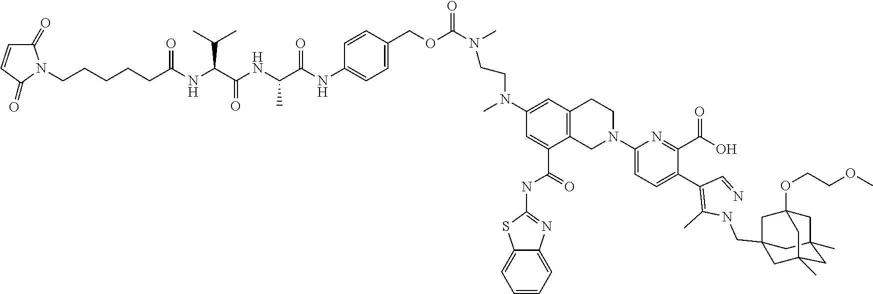 [SMILES: COCCOC12CC3(C)CC(C)(CC(CN/C(C)=C(\C=N)c4ccc(N5CCc6cc(N(C)CCN(C)C(=O)OCc7ccc(NC(=O)[C@H](C)NC(=O)[C@@H](NC(=O)CCCCCN8C(=O)C=CC8=O)C(C)C)cc7)cc(C(=O)Nc7nc8ccccc8s7)c6C5)nc4C(=O)O)(C3)C1)C2] |
| 2.45 | OZ | 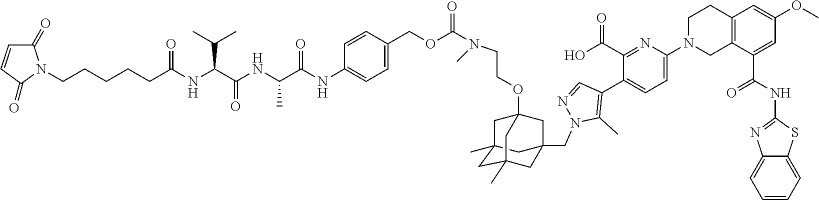 [SMILES: COc1cc2c(c(C(=O)Nc3nc4ccccc4s3)c1)CN(c1ccc(-c3cnn(CC45CC6(C)CC(C)(C4)CC(OCCN(C)C(=O)OCc4ccc(NC(=O)[C@H](C)NC(=O)[C@@H](NC(=O)CCCCCN7C(=O)C=CC7=O)C(C)C)cc4)(C6)C5)c3C)c(C(=O)O)n1)CC2] |
| 2.46 | PA | 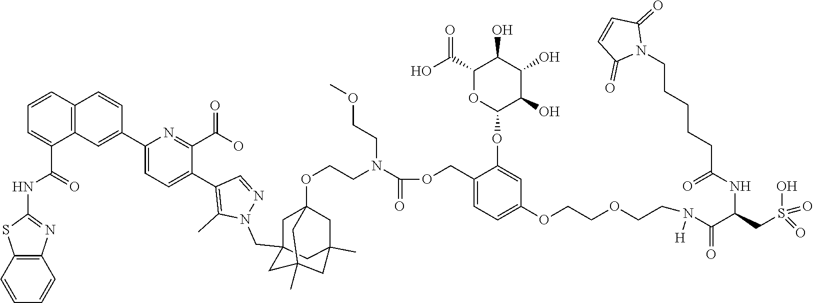 [SMILES: COCCN(CCOC12CC3(C)CC(C)(CC(Cn4ncc(-c5ccc(-c6ccc7cccc(C(=O)Nc8nc9ccccc9s8)c7c6)nc5C(=O)O)c4C)(C3)C1)C2)C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O] |
| 2.47 | QL | 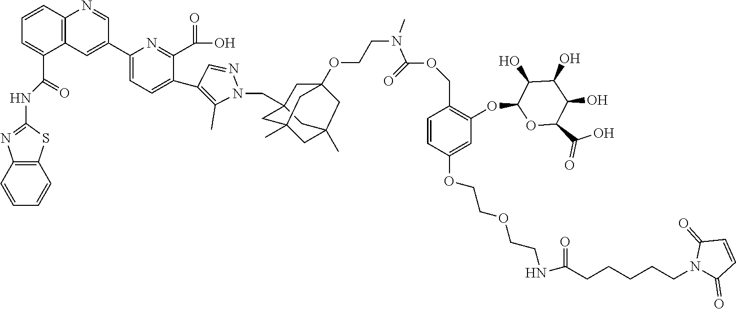 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)OCc1ccc(OCCOCCNC(=O)CCCCCN4C(=O)C=CC4=O)cc1O[C@@H]1O[C@H](C(=O)O)[C@H](O)[C@H](O)[C@@H]1O)(C3)C2] |
| 2.48 | QM | 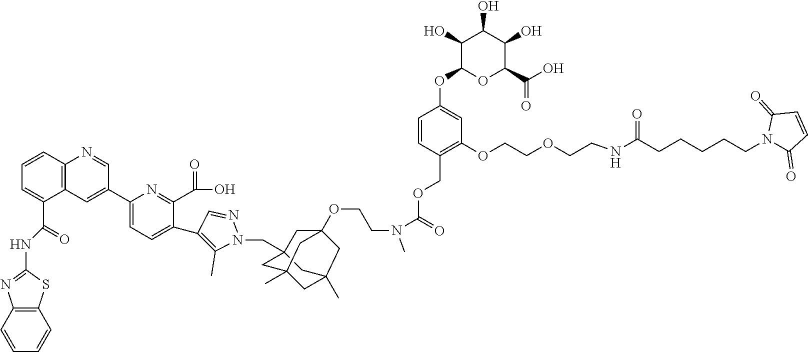 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)OCc1ccc(O[C@@H]4O[C@H](C(=O)O)[C@H](O)[C@H](O)[C@@H]4O)cc1OCCOCCNC(=O)CCCCCN1C(=O)C=CC1=O)(C3)C2] |
| 2.49 | QN | 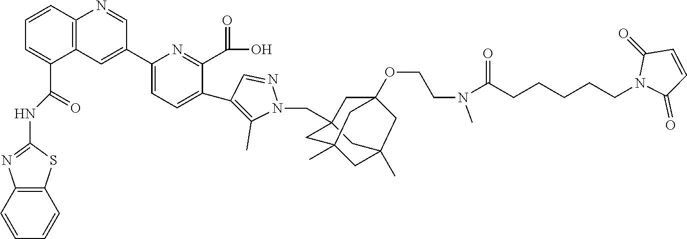 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)CCCCCN1C(=O)C=CC1=O)(C3)C2] |
| 2.50 | QT | 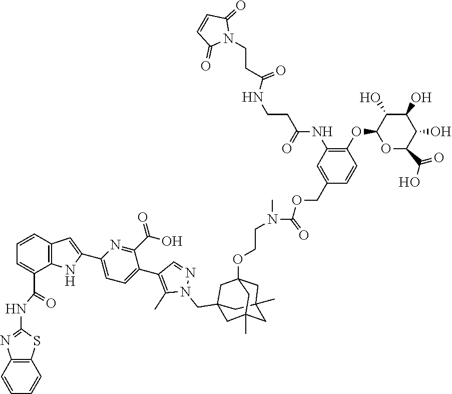 [SMILES: Cc1c(-c2ccc(-c3cc4cccc(C(=O)Nc5nc6ccccc6s5)c4[nH]3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)c(NC(=O)CCNC(=O)CCN4C(=O)C=CC4=O)c1)(C3)C2] |
| 2.51 | RF | 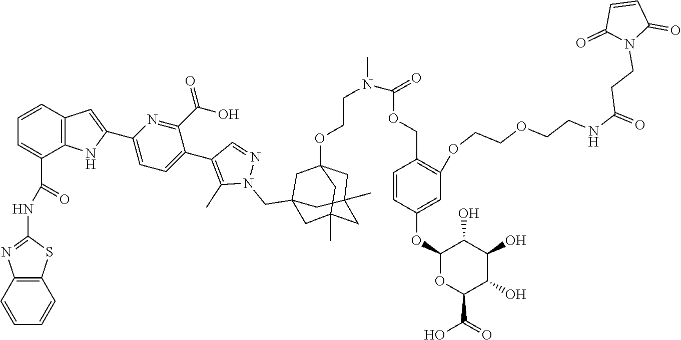 [SMILES: Cc1c(-c2ccc(-c3cc4cccc(C(=O)Nc5nc6ccccc6s5)c4[nH]3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.52 | RG | 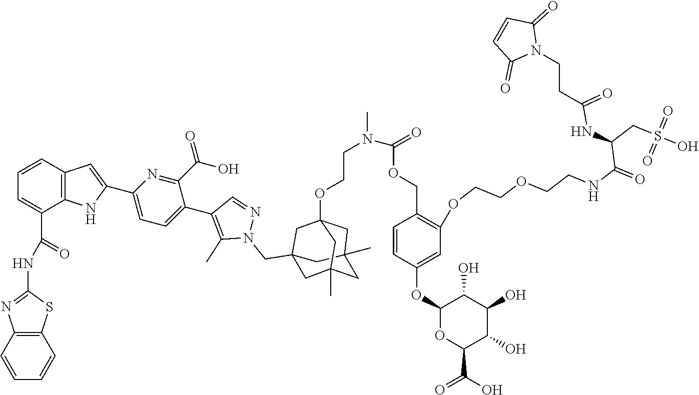 [SMILES: Cc1c(-c2ccc(-c3cc4cccc(C(=O)Nc5nc6ccccc6s5)c4[nH]3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)[C@H](CS(=O)(=O)O)NC(=O)CCN1C(=O)C=CC1=O)(C3)C2] |
| 2.53 | SF | 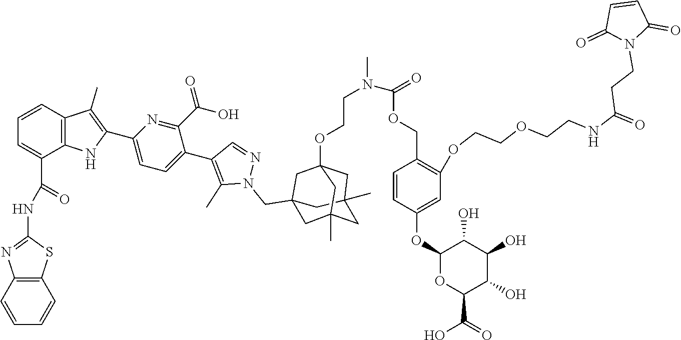 [SMILES: Cc1c(-c2ccc(-c3cnn(CC45CC6(C)CC(C)(C4)CC(OCCN(C)C(=O)OCc4ccc(O[C@@H]7O[C@H](C(=O)O)[C@@H](O)[C@H](O)[C@H]7O)cc4OCCOCCNC(=O)CCN4C(=O)C=CC4=O)(C6)C5)c3C)c(C(=O)O)n2)[nH]c2c(C(=O)Nc3nc4ccccc4s3)cccc12] |
| 2.54 | SR | 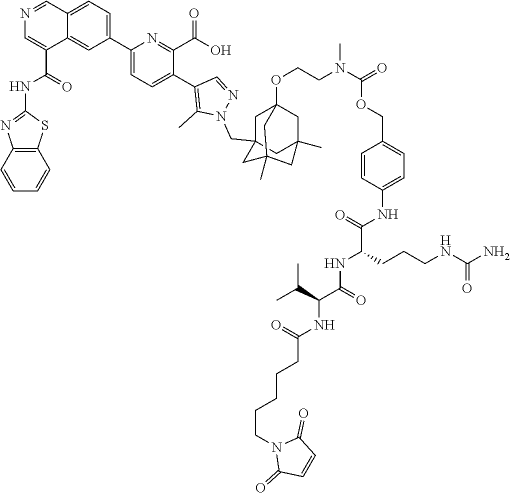 [SMILES: Cc1c(-c2ccc(-c3ccc4cncc(C(=O)Nc5nc6ccccc6s5)c4c3)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.55 | YZ | 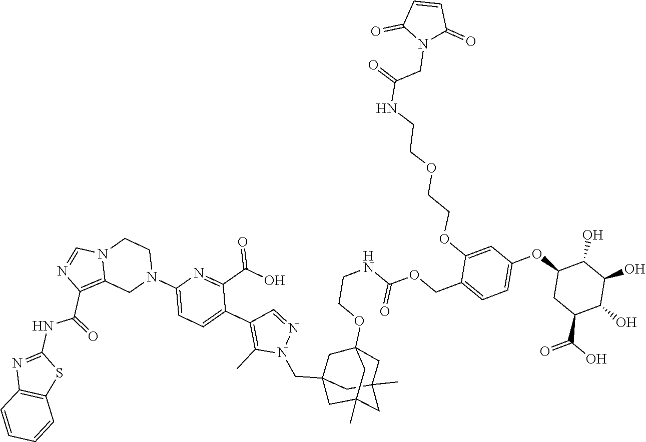 [SMILES: Cc1c(-c2ccc(N3CCn4cnc(C(=O)Nc5nc6ccccc6s5)c4C3)nc2C(=O)O)cnn1CC12CC3(C)CC(C)(C1)CC(OCCNC(=O)OCc1ccc(O[C@@H]4C[C@H](C(=O)O)[C@@H](O)[C@H](O)[C@H]4O)cc1OCCOCCNC(=O)CN1C(=O)C=CC1=O)(C3)C2] |
| 2.56 | QR | 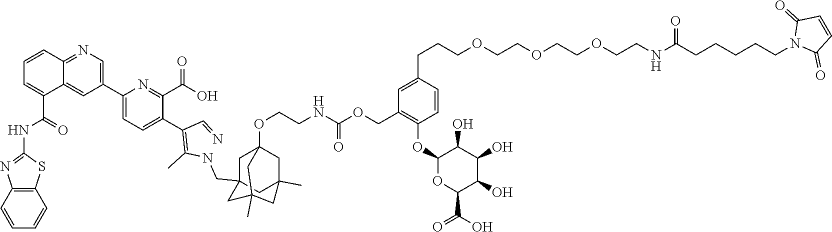 [SMILES: C/C(NCC12CC3(C)CC(C)(C1)CC(OCCNC(=O)OCc1cc(CCCOCCOCCOCCNC(=O)CCCCCN4C(=O)C=CC4=O)ccc1O[C@@H]1O[C@H](C(=O)O)[C@H](O)[C@H](O)[C@@H]1O)(C3)C2)=C(/C=N)c1ccc(-c2cnc3cccc(C(=O)Nc4nc5ccccc5s4)c3c2)nc1C(=O)O] |
| 2.57 | SE | 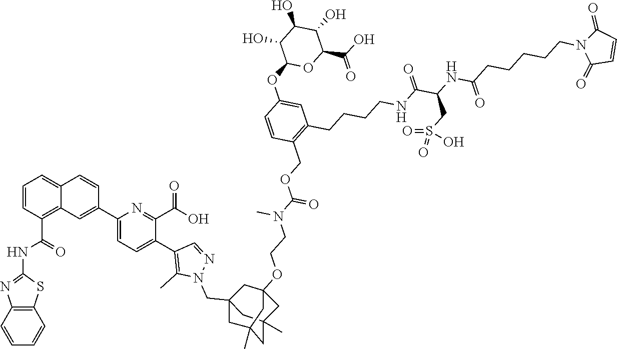 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1CCCCNC(=O)[C@H](CS(=O)(=O)O)NC(=O)CCCCCN1C(=O)C=CC1=O)(C3)C2] |
| 2.58 | UH | 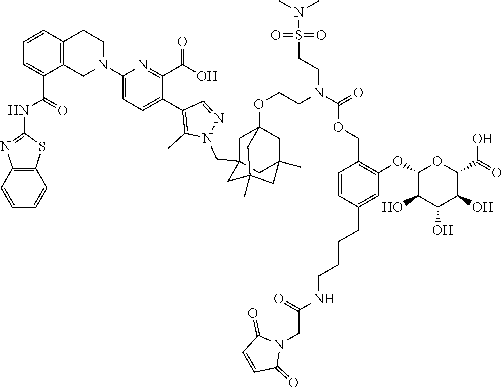 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)N(C)C)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.59 | UI | 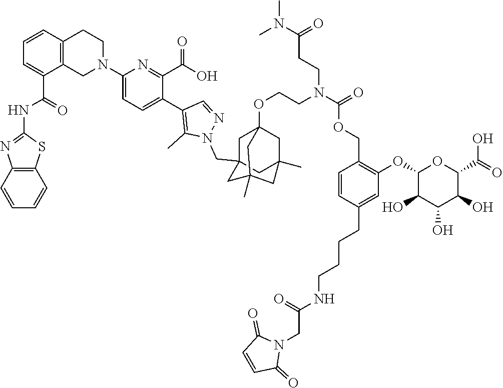 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)N(C)C)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.60 | US | 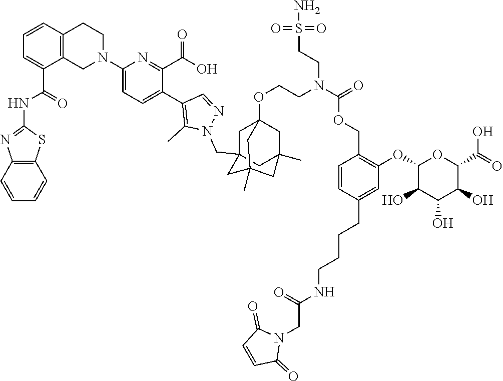 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(N)(=O)=O)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.61 | UY | 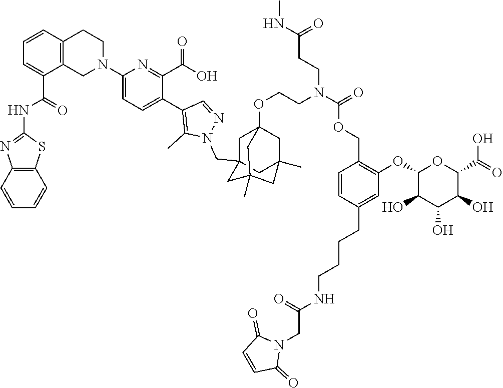 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(CCCCNC(=O)CN2C(=O)C=CC2=O)cc1O[C@@H]1O[C@H](C(=O)O)[C@@H](O)[C@H](O)[C@H]1O] |
| 2.62 | UX | 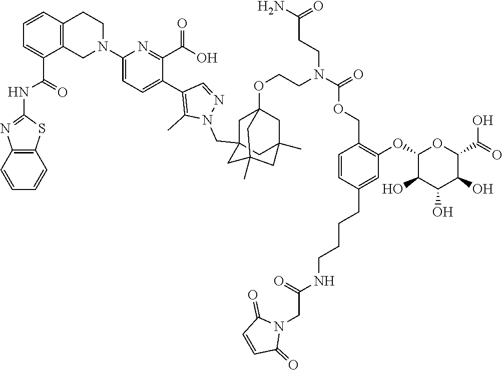 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(N)=O)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.63 | WZ | 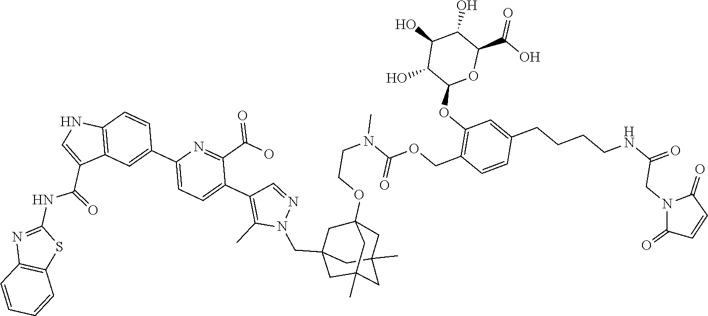 [SMILES: Cc1c(-c2ccc(-c3ccc4[nH]cc(C(=O)Nc5nc6ccccc6s5)c4c3)nc2C(=O)O)cnn1CC12CC3(C)CC(C)(C1)CC(OCCN(C)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.64 | XO | 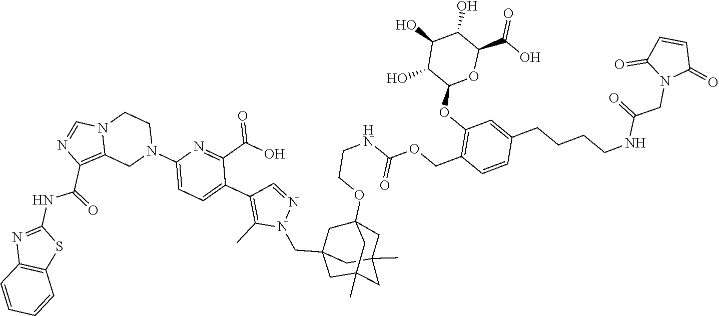 [SMILES: Cc1c(-c2ccc(N3CCn4cnc(C(=O)Nc5nc6ccccc6s5)c4C3)nc2C(=O)O)cnn1CC12CC3(C)CC(C)(C1)CC(OCCNC(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.65 | XW | 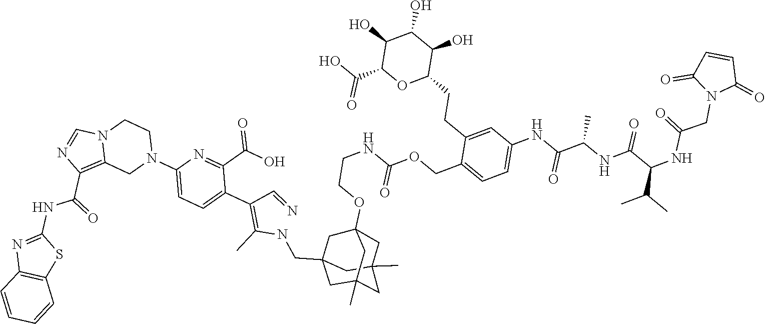 [SMILES: C/C(NCC12CC3(C)CC(C)(C1)CC(OCCNC(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2)=C(/C=N)c1ccc(N2CCn3cnc(C(=O)Nc4nc5ccccc5s4)c3C2)nc1C(=O)O] |
| 2.66 | YG | 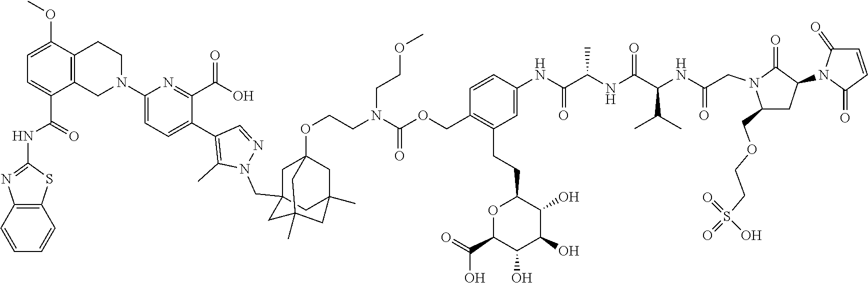 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O] |
| 2.67 | ZT | 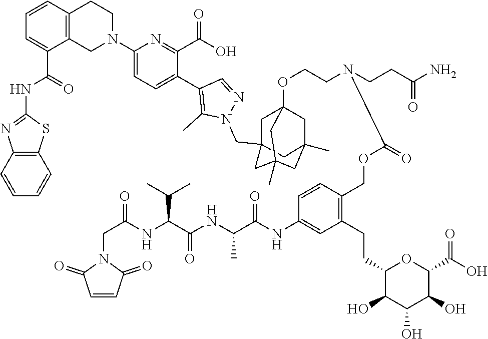 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(N)=O)C(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |
| 2.68 | AAN | 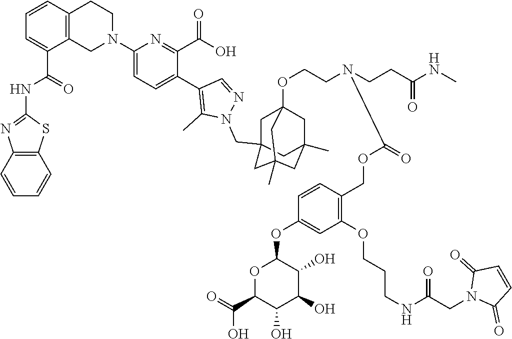 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCCNC(=O)CN1C(=O)C=CC1=O] |
| 2.69 | AAO | 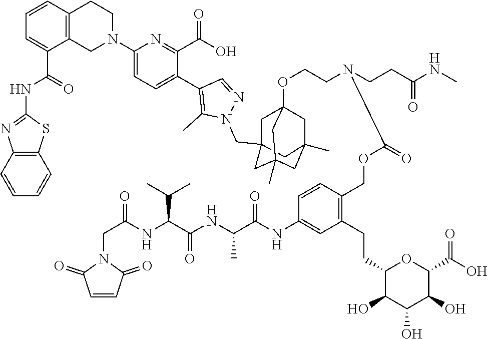 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(NC(=O)[C@H](C)NC(=O)[C@@H](NC(=O)CN2C(=O)C=CC2=O)C(C)C)cc1CC[C@@H]1O[C@H](C(=O)O)[C@@H](O)[C@H](O)[C@H]1O] |
| 2.70 | AAP | 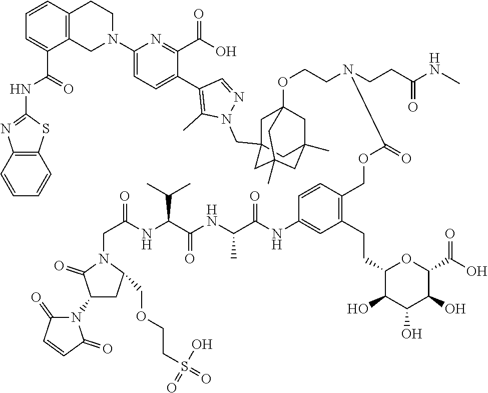 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O] |
| 2.71 | ABF | 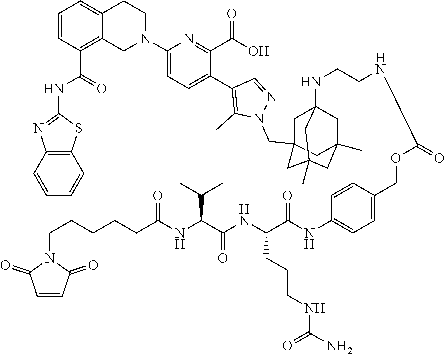 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(NCCNC(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.72 | ZZ | 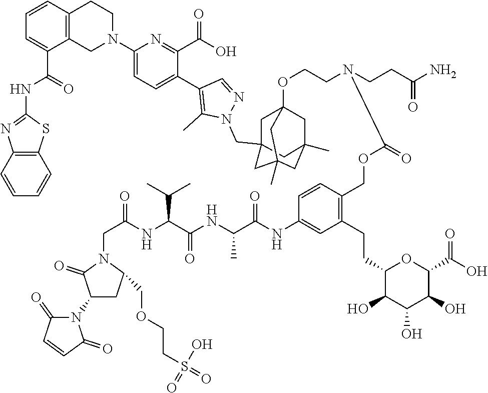 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(N)=O)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |

In certain embodiments, the synthon is selected from the group consisting of synthon examples 2.1, 2.2, 2.3, 2.4, 2.5, 2.6, 2.7, 2.8, 2.9, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, and pharmaceutically acceptable salts thereof.

In certain embodiments, the ADC, or a pharmaceutically acceptable salt thereof, is formed by contacting an antibody that binds a cell surface receptor or tumor associated antigen expressed on a tumor cell with a synthon under conditions in which the synthon covalently links to the antibody, wherein the synthons is selected from the group consisting of synthon examples 2.1, 2.2, 2.3, 2.4, 2.5, 2.6, 2.7, 2.8, 2.9, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, and 2.72.

### 4.8. Antibody Drug Conjugates

Bcl-xL inhibitory activity of ADCs described herein may be confirmed in cellular assays with appropriate target cells and/or in vivo assays. Specific assays that may be used to confirm activity of ADCs that target EGFR EpCAM or NCAM1 are provided in Examples 7 and 8. Generally, ADCs will exhibit an EC₅₀ of less than about 100 nM in such a cellular assay, although the ADCs may exhibit significantly lower EC₅₀s, for example, less than about 10, 5, or even 1 nM. Similar cellular assays with cells expressing specific target antigens may be used to confirm the Bcl-xL inhibitory activity of ADCs targeting other antigens.

### 4.9. Methods of Synthesis

The Bcl-xL inhibitors and synthons described herein may be synthesized using standard, known techniques of organic chemistry. General schemes for synthesizing Bcl-xL inhibitors and synthons that may be used as-is or modified to synthesize the full scope of Bcl-xL inhibitors and synthons described herein are provided below. Specific methods for synthesizing exemplary Bcl-xL inhibitors and synthons that may be useful for guidance are provided in the Examples section.

ADCs may likewise be prepared by standard methods, such as methods analogous to those described in Hamblett et al., 2004. “Effects of Drug Loading on the Antitumor Activity of a Monoclonal Antibody Drug Conjugate”, Clin. Cancer Res. 10:7063-7070; Doronina et al., 2003, “Development of potent and highly efficacious monoclonal antibody auristatin conjugates for cancer therapy.” Nat. Biotechnol. 2 (7):778-784; and Francisco et al., 2003, “cAClO-vcMMAE, an anti-CD30-monomethylauristatin E conjugate with potent and selective antitumor activity,” Blood 102:1458-1465. For example, ADCs with four drugs per antibody may be prepared by partial reduction of the antibody with an excess of a reducing reagent such as DTT or TCEP at 37° C. for 30 min, then the buffer exchanged by elution through SEPHADEX® G-25 resin with 1 mM DTPA in DPBS. The eluent is diluted with further DPBS, and the thiol concentration of the antibody may be measured using 5,5′-dithiobis(2-nitrobenzoic acid) [Ellman's reagent]. An excess, for example 5-fold, of a linker-drug synthon is added at 4° C. for 1 hour, and the conjugation reaction may be quenched by addition of a substantial excess, for example 20-fold, of cysteine. The resulting ADC mixture may be purified on SEPHADEX G-25 equilibrated in PBS to remove unreacted synthons, desalted if desired, and purified by size-exclusion chromatography. The resulting ADC may then be then sterile-filtered, for example, through a 0.2 μm filter, and lyophilized if desired for storage. In certain embodiments, all of the interchain cysteine disulfide bonds are replaced by linker-drug conjugates. One embodiment pertains to a method of making an ADC, comprising contacting a synthon described herein with an antibody under conditions in which the synthon covalently links to the antibody.

Specific methods for synthesizing exemplary ADCs that may be used to synthesize the full range of ADCs described herein are provided in the Examples section.

### 4.9.1. General Methods for Synthesizing Bcl-xL Inhibitors

In the schemes below, the various substituents Ar¹, Ar², Z¹, R⁴, R¹⁰, R^{11a} and R^{11b} as defined in the Detailed Description section.

### 4.9.1.1. Synthesis of Compound (9)

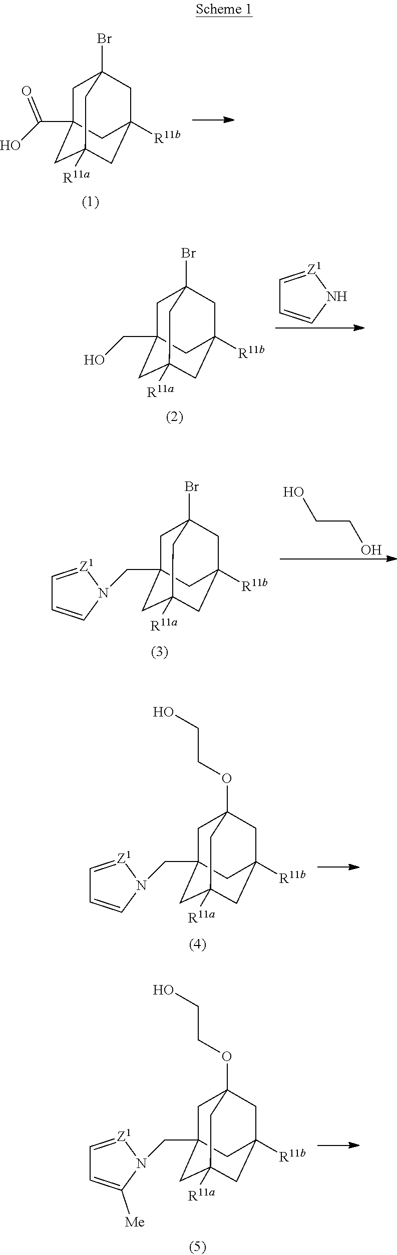

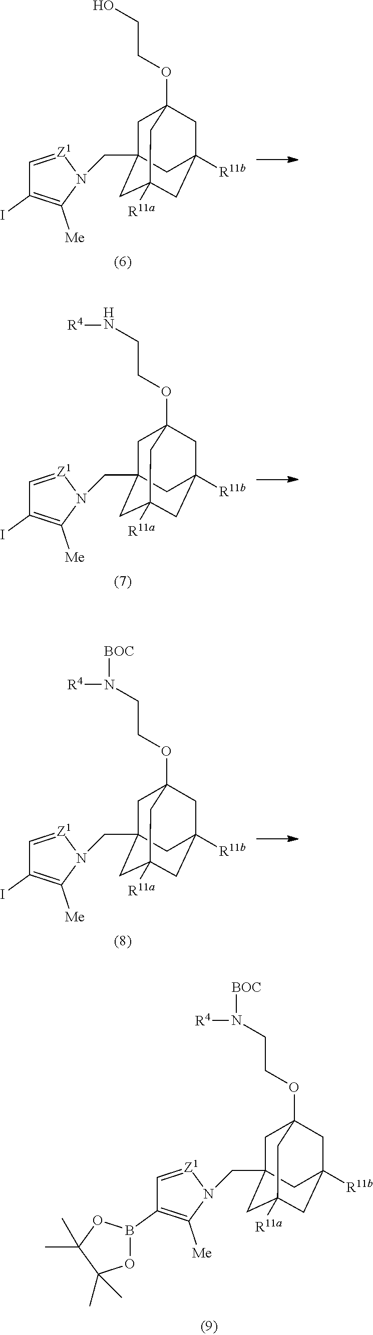

The synthesis of compound (9) is described in Scheme 1. Compound (1) can be treated with BH₃.THF to afford compound (2). The reaction is typically performed at ambient temperature in a solvent, such as, but not limited to, tetrahydrofuran. Compound (3) can be prepared by treating compound (2) with

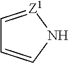

in the presence of cyanomethylenetributylphosphorane. The reaction is typically performed at an elevated temperature in a solvent such as, but not limited to, toluene. Compound (3) can be treated with ethane-1,2-diol in the presence of a base such as, but not limited to, triethylamine, to provide compound (4). The reaction is typically performed at an elevated temperature, and the reaction may be performed under microwave conditions. Compound (4) can be treated with a strong base, such as, but not limited to, n-butyllithium, followed by the addition of iodomethane, to provide compound (5). The addition and reaction is typically performed in a solvent such as, but not limited to, tetrahydrofuran, at a reduced temperature before warming up to ambient temperature for work up. Compound (5) can be treated with N-iodosuccinimide to provide compound (6). The reaction is typically performed at ambient temperature is a solvent such as, but not limited to, N,N-dimethylformamide. Compound (7) can be prepared by reacting compound (6) with methanesulfonyl chloride, in the presence of a base such as, but not limited to, triethylamine, followed by the addition of NHR⁴. The reaction with methanesulfonyl chloride is typically performed at low temperature, before increasing the temperature for the reaction with NHR⁴, and the reaction is typically performed in a solvent such as, but not limited to tetrahydrofuran. Compound (7) can be reacted with di-tert-butyl dicarbonate in the presence of 4-dimethylaminopyridine to provide compound (8). The reaction is typically performed at ambient temperature in a solvent such as, but not limited to tetrahydrofuran. The borylation of compound (8) to provide compound (9) can be performed under conditions described herein and readily available in the literature.

### 4.9.1.2. Synthesis of Compound (12)

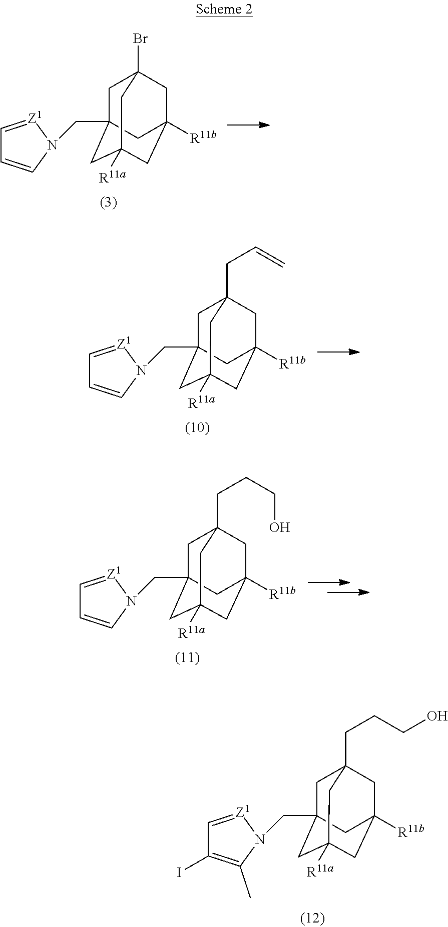

The synthesis of intermediate (12) is described in Scheme 2. Compound (3) can be treated with tri-n-butyl-allylstannane in the presence of ZnCl₂.Et₂O or N, N′-azoisobutyronitrile (AIBN) to provide compound (10) (Yamamoto et al., 1998, Heterocycles 47:765-780). The reaction is typically performed at −78° C. in a solvent, such as, but not limited to dichloromethane. Compound (10) can be treated under standard conditions known in the art for hydroboration/oxidation to provide compound (11). For example, treatment of compound (10) with a reagent such as BH₃.THF in a solvent such as, but not limited to, tetrahydrofuran followed by treatment of the intermediate alkylborane adduct with an oxidant such as, but not limited to, hydrogen peroxide in the presence of a base such as, but not limited to, sodium hydroxide would provide compound (11) (Brown et al., 1968, J. Am. Chem. Soc., 86:397). Typically the addition of BH₃.THF is performed at low temperature before warming to ambient temperature, which is followed by the addition of hydrogen peroxide and sodium hydroxide to generate the alcohol product. Compound (12) can be generated according to Scheme 1, as previously described for compound (9).

### 4.9.1.3. Synthesis of Compound (15)

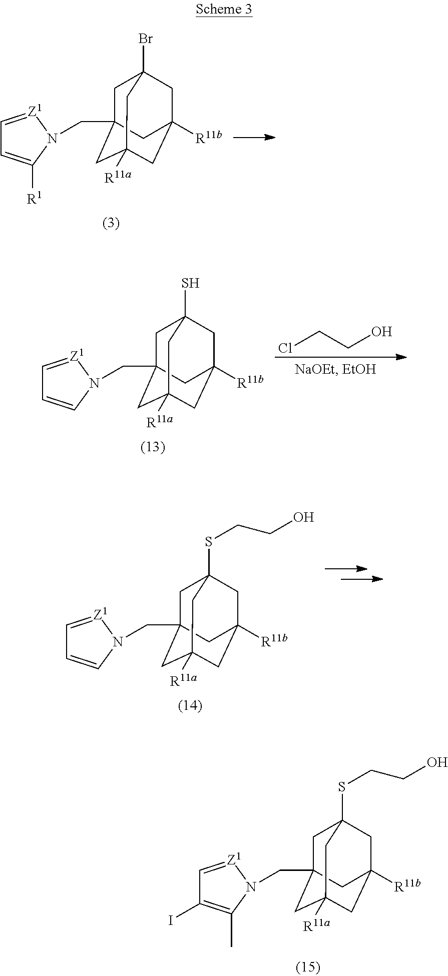

The synthesis of intermediate (15), is described in Scheme 3. Compound (3) can be reacted with thiourea in a solvent mixture of acetic acid and 48% aqueous HBr solution at 100° C. to yield an intermediate that can be subsequently treated with sodium hydroxide in a solvent mixture such as, but not limited to, 20% v/v ethanol in water to provide compound (13). Compound (13) can be reacted with 2-chloroethanol in the presence of a base such as, but not limited to, sodium ethoxide to provide compound (14). The reaction is typically performed at ambient or elevated temperatures in a solvent such as, but not limited to, ethanol. Compound (15) can be generated according to Scheme 1, as previously described for compound (9).

### 4.9.1.4. Synthesis of Compound (22)

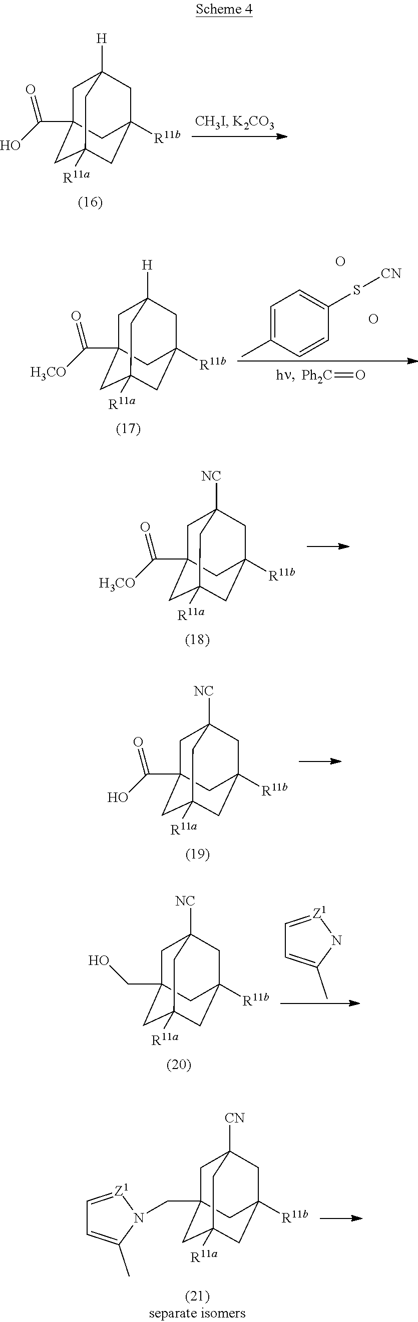

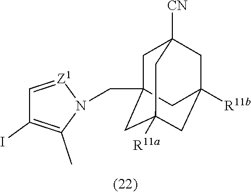

The synthesis of compound (22) is described in Scheme 4. Compound (16) can be reacted with iodomethane in the presence of a base such as, but not limited to, potassium carbonate to provide compound (17). The reaction is typically conducted at ambient or elevated temperature in a solvent such as, but not limited to, acetone or N,N-dimethylformamide. Compound (17) can be reacted under photochemical conditions with tosyl cyanide in the presence of benzophenone to provide compound (18) (see Kamijo et al., Org. Lett., 2011, 13:5928-5931). The reaction is typically run at ambient temperature in a solvent such as, but not limited to, acetonitrile or benzene using a Riko 100W medium pressure mercury lamp as the light source. Compound (18) can be reacted with lithium hydroxide in a solvent system such as, but not limited to, mixtures of water and tetrahydrofuran or water and methanol to provide compound (19). Compound (19) can be treated with BH₃.THF to provide compound (20). The reaction is typically performed at ambient temperature in a solvent, such as, but not limited to, tetrahydrofuran. Compound (21) can be prepared by treating compound (20) with

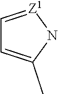

in the presence of cyanomethylenetributylphosphorane. The reaction is typically performed at an elevated temperature in a solvent such as, but not limited to, toluene. Compound (21) can be treated with N-iodosuccinimide to provide compound (22). The reaction is typically performed at ambient temperature is a solvent such as, but not limited to, N,N-dimethylformamide.

### 4.9.1.5. Synthesis of Compound (24)

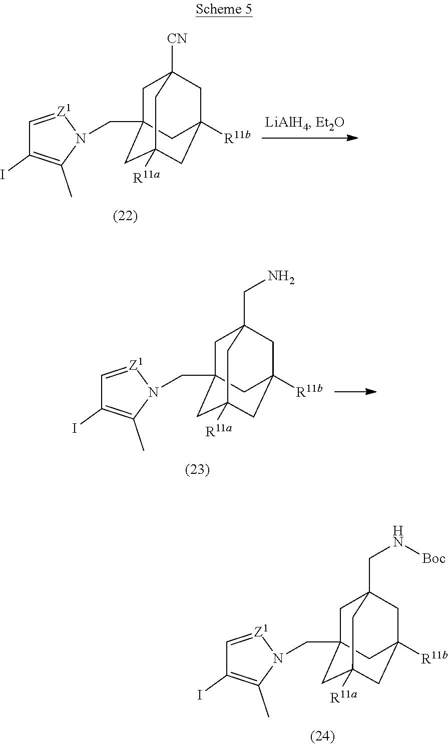

The synthesis of compound (24) is described in Scheme 5. Compound (22) can be treated with a reducing agent such as, but not limited to, lithium aluminum hydride in a solvent such as, but not limited to, diethyl ether or tetrahydrofuran to provide compound (23). Typically the reaction is performed at 0° C. before warming to ambient or elevated temperature. Compound (23) can be reacted with di-tert-butyl dicarbonate under standard conditions described herein or in the literature to provide compound (24).

### 4.9.1.6. Synthesis of Compound (24a)

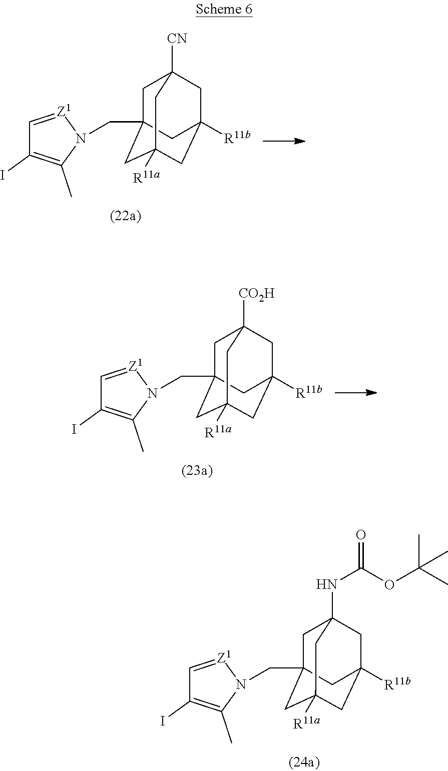

The synthesis of intermediate (24a) is described in Scheme 6. Compound (22a) can be hydrolyzed using conditions described in the literature to provide compound (23a). Typically the reaction is run in the presence of potassium hydroxide in a solvent such as, but not limited to, ethylene glycol at elevated temperatures (see Roberts et al., 1994, J. Org. Chem., 1994, 59:6464-6469; Yang et al, 2013, Org. Lett., 15:690-693). Compound (24a) can be made from compound (23a) by Curtius rearrangement using conditions described in the literature. For example, compound (23a) can be reacted with sodium azide in the presence of tetrabutylammonium bromide, zinc(II) triflate and di-tert-butyl dicarbonate to provide compound (24a) (see Lebel et al., Org. Lett., 2005, 7:4107-4110). Typically the reaction is run at elevated temperatures, preferably from 40-50° C. in a solvent such as, but not limited to, tetrahydrofuran.

### 4.9.1.7. Synthesis of Compound (29)

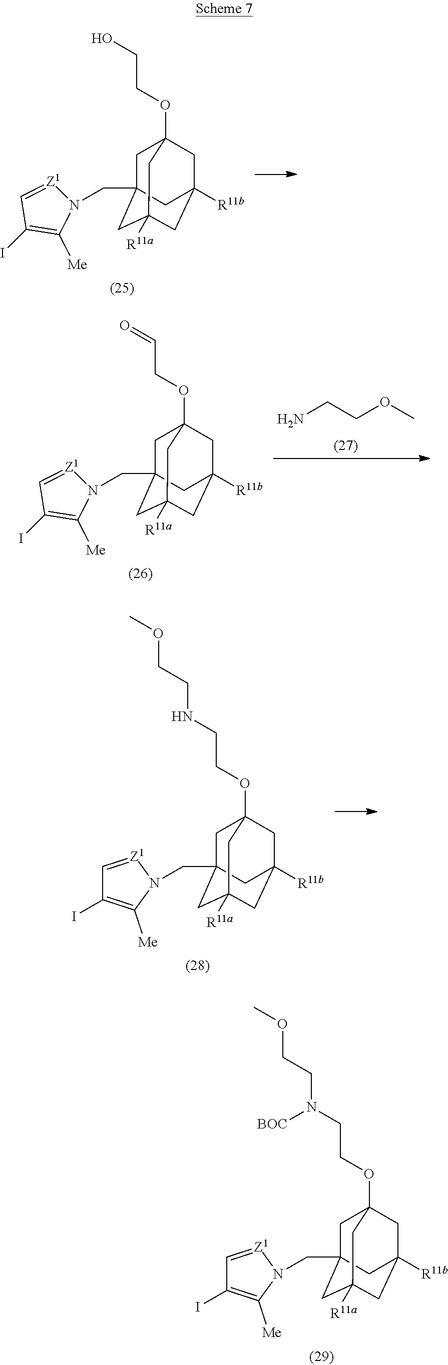

Scheme 7 describes a functionalization of the adamantane ring substituent. Dimethyl sulfoxide can be reacted with oxalyl chloride, followed by the addition of compound (25), in the presence of a base such as, but not limited to triethylamine, to provide compound (26). The reaction is typically performed at low temperature in a solvent such as, but not limited to, dichloromethane. Compound (27) can be reacted with compound (26), followed by treatment with sodium borohydride, to provide compound (28). The reaction is typically performed at ambient temperature in a solvent such as, but not limited to, dichloromethane, methanol, or mixtures thereof. Compound (29) can be prepared by reacting compound (28) with di-tert-butyl dicarbonate, in the presence of N,N-dimethylpyridin-4-amine. The reaction is typically performed at ambient temperature in a solvent such as, but not limited to, tetrahydrofuran.

### 4.9.1.8. Synthesis of Compound (35)

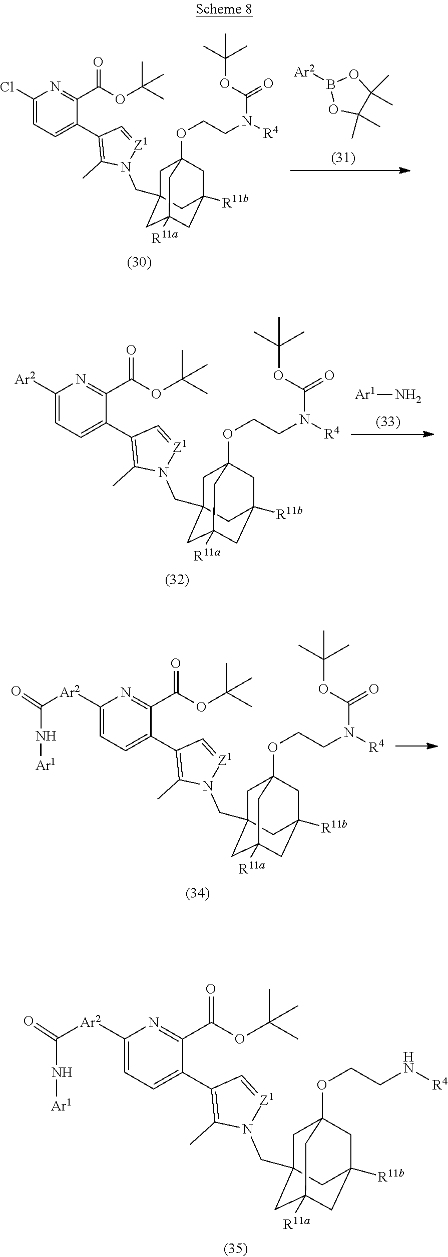

As shown in Scheme 8, compound (30), can be reacted with compound (31) under Suzuki coupling conditions described herein and readily available in the literature, to provide compound (32). Compound (34) can be prepared by reacting compound (32) with compound (33) under conditions described herein, and readily available in the literature. Compound (35) can be prepared by treating compound (34) with an acid such as, but not limited to, trifluoroacetic acid. The reaction is typically performed at ambient temperature in a solvent such as, but not limited to, dichloromethane.

### 4.9.1.9. Synthesis of Compound (43)

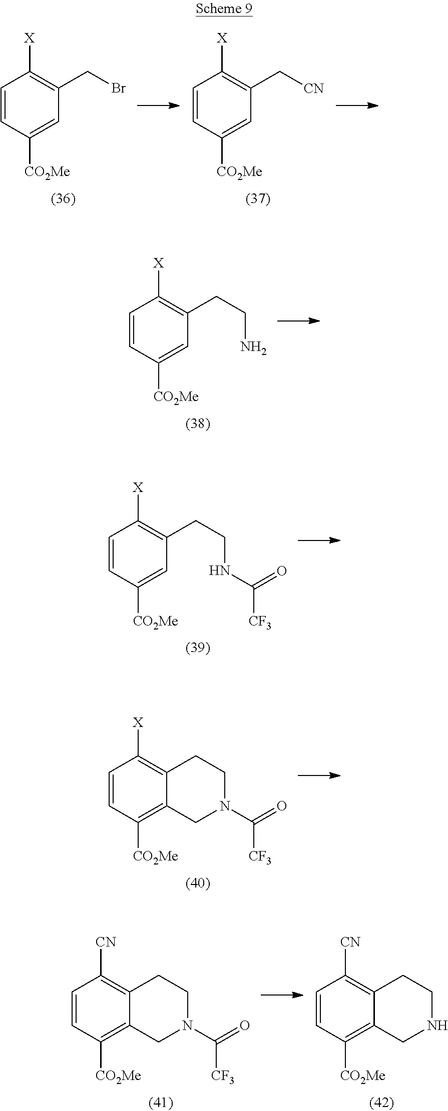

Scheme 9 describes the synthesis of substituted 1,2,3,4-tetrahydroisoquinoline intermediates. Trimethylsilanecarbonitrile can be treated with tetrabutylammonium fluoride and then reacted with compound (36), wherein X is Br or I, to provide compound (37). The additions are typically performed at ambient temperature before heating to an elevated temperature, in a solvent such as, but not limited to, tetrahydrofuran, acetonitrile, or mixtures thereof. Compound (37) can be treated with borane to provide compound (38). The reaction is typically performed at ambient temperature in a solvent such as, but not limited to, tetrahydrofuran. Compound (39) can be prepared by treating compound (38) with trifluoroacetic anhydride, in the presence of a base such as, but no limited to, triethylamine. The reaction is initially performed at low temperature before warming to ambient temperature in a solvent such as, but not limited to, dichloromethane. Compound (39) can be treated with paraformaldehyde in the presence of sulfuric acid to provide compound (40). The reaction is typically performed at ambient temperature. Compound (41) can be prepared by reacting compound (40) with dicyanozinc in the presence of a catalyst such as, but not limited to, tetrakis(triphenylphosphine)palladium(0). The reaction is typically performed at an elevated temperature under a nitrogen atmosphere in a solvent such as, but not limited to, N,N-dimethylformamide. Compound (41) can be treated with potassium carbonate to provide compound (42). The reaction is typically performed at ambient temperature in a solvent such as, but not limited to, methanol, tetrahydrofuran, water, or mixtures thereof.

### 4.9.1.10. Synthesis of Compound (47)

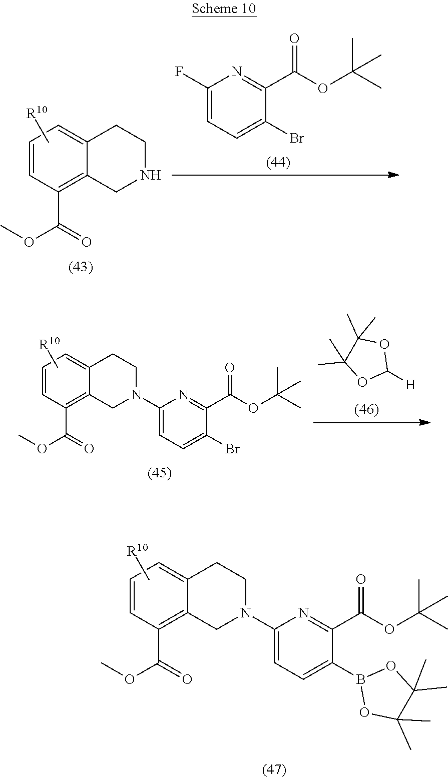

As shown in Scheme 10, compound (45) can be prepared by reacting compound (43), with tert-butyl 3-bromo-6-fluoropicolinate (44) in the presence of a base, such as, but not limited to, N,N-diisopropylethylamine or triethylamine. The reaction is typically performed under an inert atmosphere at an elevated temperature, in a solvent, such as, but not limited to, dimethyl sulfoxide. Compound (45) can be reacted with 4,4,5,5-tetramethyl-1,3,2-dioxaborolane (46), under borylation conditions described herein or in the literature to provide compound (47).

### 4.9.1.11. Synthesis of Compound (53)

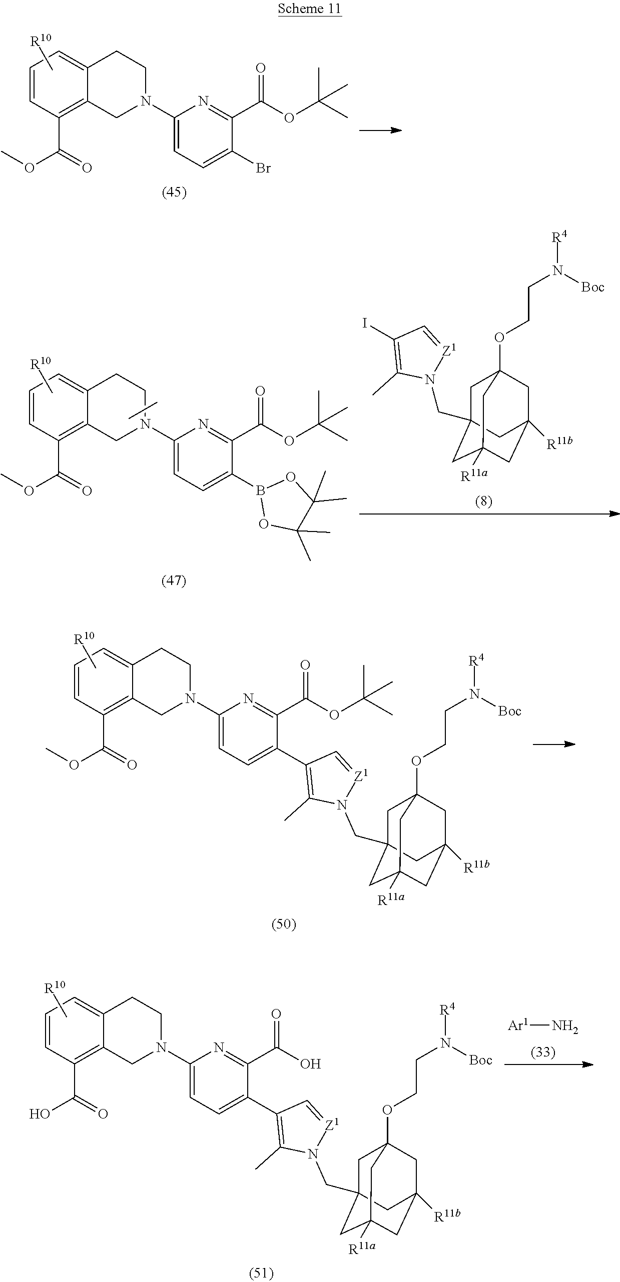

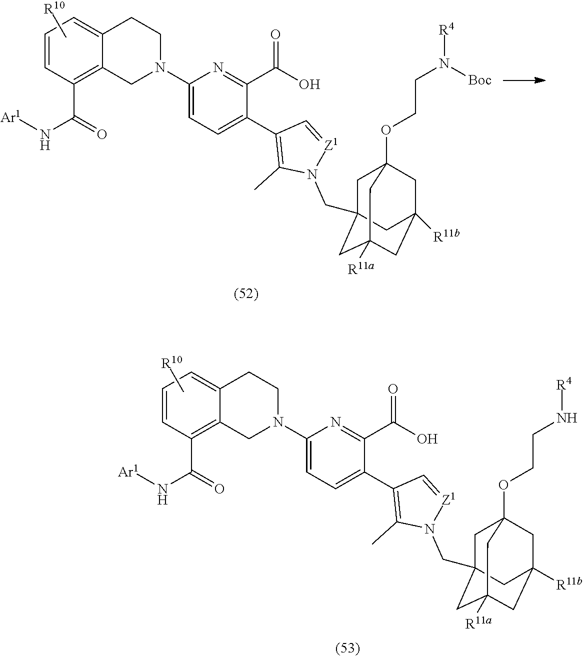

Scheme 11 describes the synthesis of optionally substituted 1,2,3,4-tetrahydroisoquinoline Bcl-xL inhibitors. Compound (47) can be prepared by reacting compound (45) with pinacolborane, in the presence of a base such as but not limited to triethylamine, and a catalyst such as but not limited to [1,1′-bis(diphenylphosphino)ferrocene]dichloropalladium(II). The reaction is typically performed at an elevated temperature in a solvent such as, but not limited to acetonitrile. Compound (50) can be prepared by reacting compound (47) with compound (8) under Suzuki coupling conditions described herein and readily available in the literature. Compound (50) can be treated with lithium hydroxide to provide compound (51). The reaction is typically performed at ambient temperature in a solvent such as, but not limited to, tetrahydrofuran, methanol, water, or mixtures thereof. Compound (51) can be reacted with compound (33) under amidation conditions described herein and readily available in the literature to provide compound (52). Compound (53) can be prepared by treating compound (52) with an acid such as, but not limited to, trifluoroacetic acid. The reaction is typically performed at ambient temperature in a solvent such as, but not limited to, dichloromethane.

### 4.9.1.12. Synthesis of Compound (66)

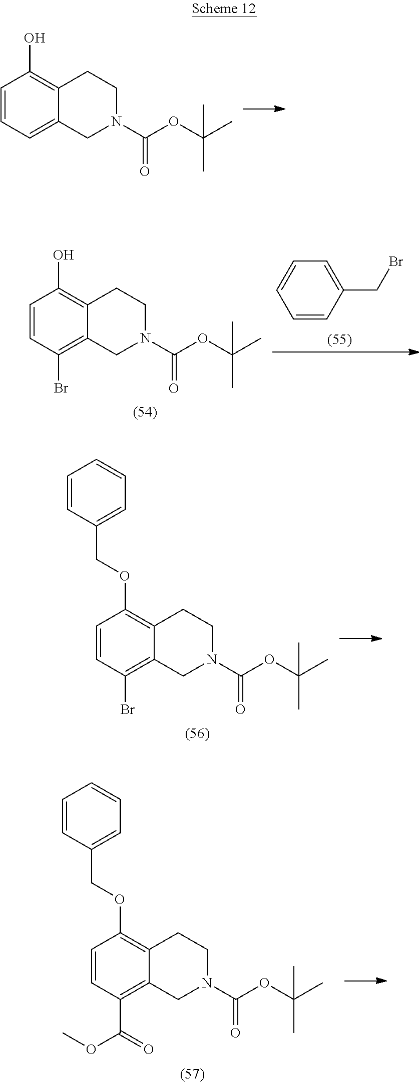

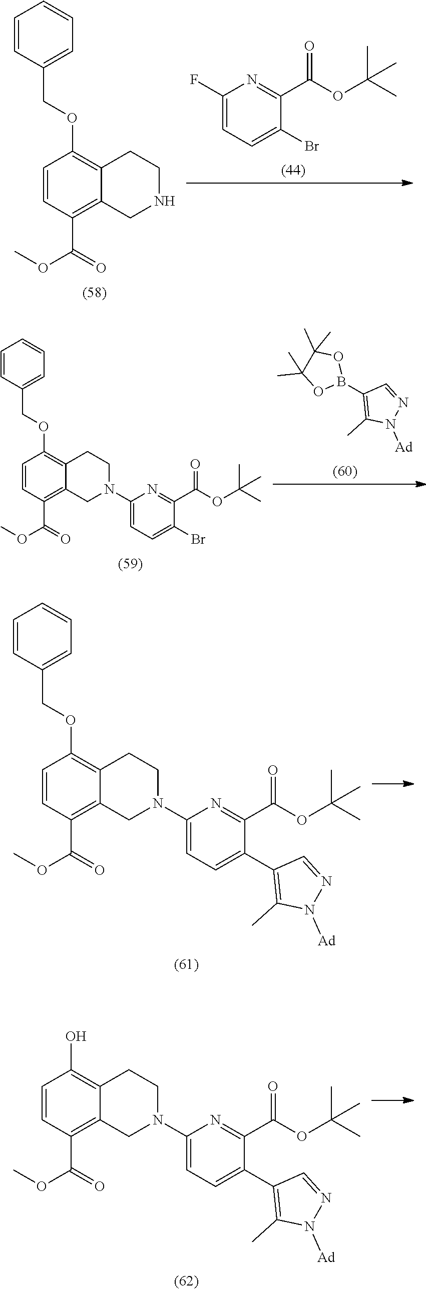

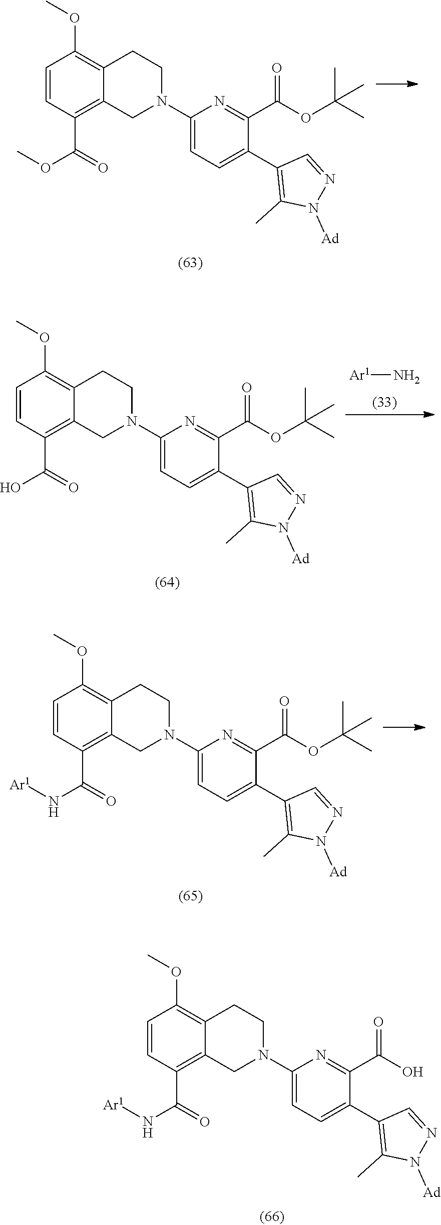

Scheme 12 describes the synthesis of 5-methoxy 1,2,3,4-tetrahydroisoquinoline Bcl-xL inhibitors. tert-Butyl 8-bromo-5-hydroxy-3,4-dihydroisoquinoline-2(1H)-carboxylate (54) can be prepared by treating tert-butyl 5-hydroxy-3,4-dihydroisoquinoline-2(1H)-carboxylate with N-bromosuccinimide. The reaction is typically performed at ambient temperature in a solvent such as, but not limited to N,N-dimethylformamide. Butyl 8-bromo-5-hydroxy-3,4-dihydroisoquinoline-2(1H)-carboxylate (54) can be reacted with benzyl bromide (55) in the presence of a base such as, but not limited to, potassium carbonate to provide tert-butyl 5-(benzyloxy)-8-bromo-3,4-dihydroisoquinolin-2(1H)-carboxylate (56). The reaction is typically performed at an elevated temperature in a solvent such as, but not limited to, acetone. tert-Butyl 5-(benzyloxy)-8-bromo-3,4-dihydroisoquinoline-2(1H)-carboxylate (56) can be treated with carbon monoxide in the presence of methanol and a base such as, but not limited to, triethylamine, and a catalyst such as but not limited to [1,1′-bis(diphenylphosphino)ferrocene]dichloropalladium(II), to provide 2-tert-butyl 8-methyl 5-(benzyloxy)-3,4-dihydroisoquinoline-2,8(1H)-dicarboxylate (57). The reaction is typically performed at an elevated temperature. Methyl 5-(benzyloxy)-1,2,3,4-tetrahydroisoquinoline-8-carboxylate (58) can be prepared by treating 2-tert-butyl 8-methyl 5-(benzyloxy)-3,4-dihydroisoquinoline-2,8(1H)-dicarboxylate (57) with hydrochloric acid. The reaction is typically performed at ambient temperature, in a solvent such as, but not limited to, tetrahydrofuran, dioxane, or mixtures thereof. Methyl 5-(benzyloxy)-1,2,3,4-tetrahydroisoquinoline-8-carboxylate (58) can be reacted with tert-butyl 3-bromo-6-fluoropicolinate (44) in the presence of a base such as, but not limited to, triethylamine, to provide methyl 5-(benzyloxy)-2-(5-bromo-6-(tert-butoxycarbonyl)pyridin-2-yl)-1,2,3,4-tetrahydroisoquinoline-8-carboxylate (59). The reaction is typically performed at elevated temperature in a solvent such as, but not limited to, dimethyl sulfoxide. Methyl 5-(benzyloxy)-2-(5-bromo-6-(tert-butoxycarbonyl)pyridin-2-yl)-1,2,3,4-tetrahydroisoquinoline-8-carboxylate (59) can be reacted with compound (60), wherein Ad is a methyladamantane moiety of the compounds of the disclosure (e.g., the compounds of formula (IIa) and (IIb)) under Suzuki coupling conditions described herein and readily available in the literature, to provide compound (61). Compound (61) can be treated with hydrogen gas in the presence of palladium hydroxide to provide compound (62). The reaction is typically performed at elevated temperature in a solvent such as, but not limited to, tetrahydrofuran. Compound (63) can be prepared by reacting compound (62) with (trimethylsilyl)diazomethane. The reaction is typically performed at ambient temperature, in a solvent such as, but not limited to, dichloromethane, methanol, diethyl ether, or mixtures thereof. Compound (63) can be treated with lithium hydroxide to provide compound (64). The reaction is typically performed at ambient temperature in a solvent such as, but not limited to, tetrahydrofuran, methanol, water, or mixtures thereof. Compound (64) can be reacted with compound (33) under amidation conditions described herein and readily available in the literature to provide compound (65). Compound (66) can be prepared by treating compound (65) with hydrochloric acid. The reaction is typically performed at ambient temperature in a solvent such as, but not limited to, dioxane.

### 4.9.2. General Methods for Synthesizing Synthons

In the schemes below, the various substituents Ar¹, Ar², Z¹, R⁴, R^{11a} and R^{11b} are as defined in the Detailed Description section.

### 4.9.2.1. Synthesis of Compound (89)

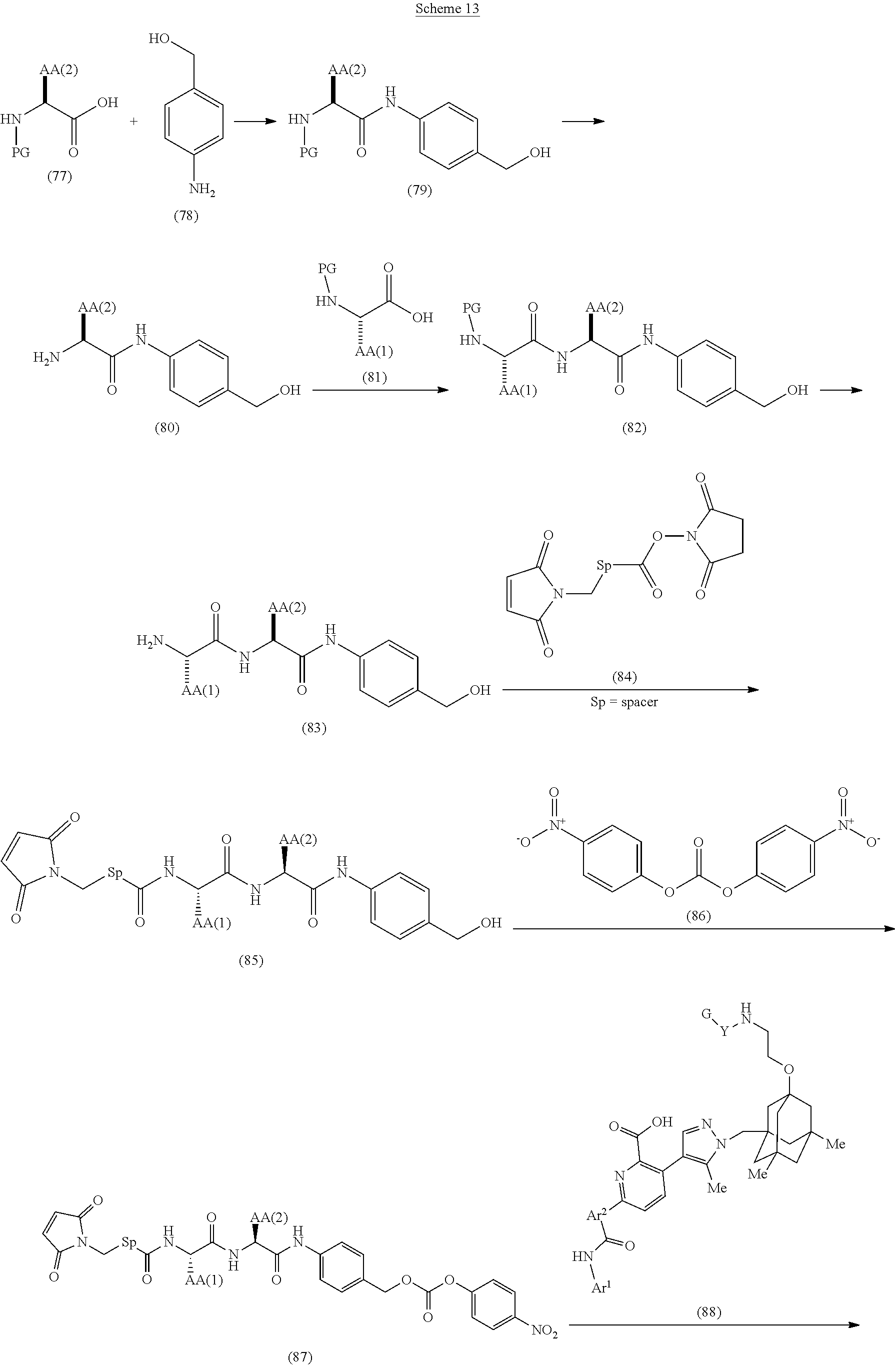

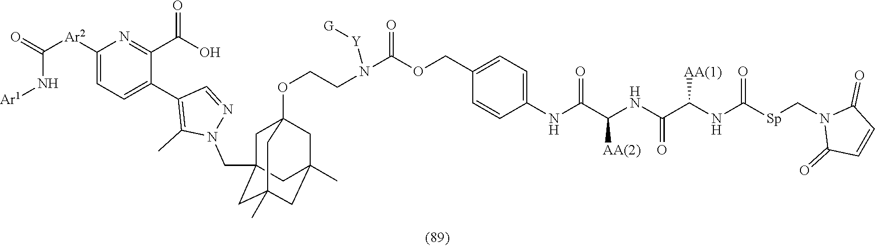

As shown in scheme 13, compounds of formula (77), wherein PG is an appropriate base labile protecting group and AA(2) is Cit, Ala, or Lys, can be reacted with 4-(aminophenyl)methanol (78), under amidation conditions described herein or readily available in the literature to provide compound (79). Compound(80) can be prepared by reacting compound (79) with a base such as, but not limited to, diethylamine. The reaction is typically performed at ambient temperature in a solvent such as but not limited to N,N-dimethylformamide. Compound (8), wherein P is an appropriate compound (83) to provide compound (85). The reaction is typically performed at ambient temperature in a solvent such as but not limited to N,N-dimethylformamide. Compound (85) can be reacted with bis(4-nitrophenyl) carbonate (86) in the presence of a base such as, but not limited to N,N-diisopropylethylamine, to provide compounds (87). The reaction is typically performed at ambient temperature in a solvent such as but not limited to N,N-dimethylformamide. Compounds (87) can be reacted with compounds of formula (88) in the presence of a base such as, but not limited to, N,N-diisopropylethylamine, to provide compound (89). The reaction is typically performed at ambient temperature in a solvent such as, but not limited to, N,N-dimethylformamide.

### 4.9.2.2. Synthesis of Compounds (94) and (96)

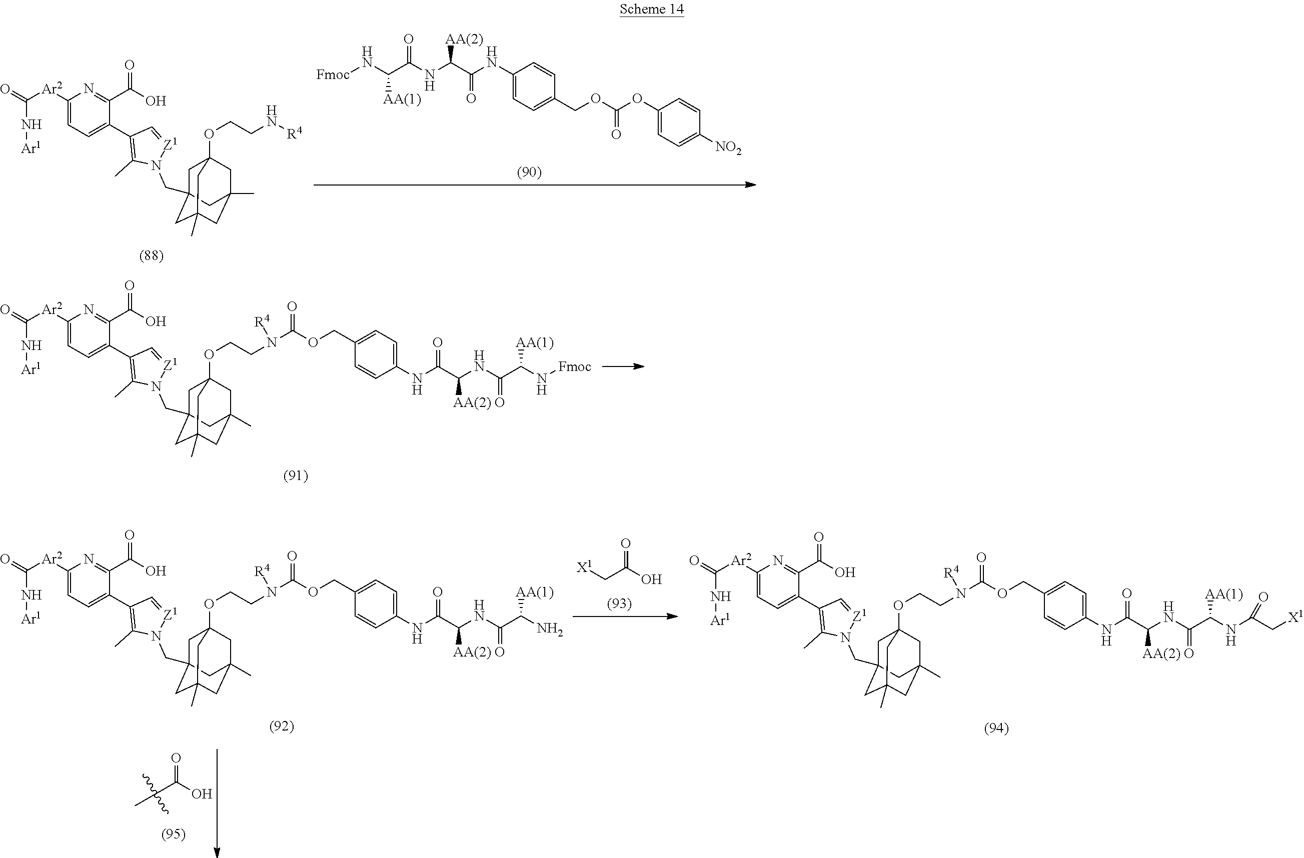

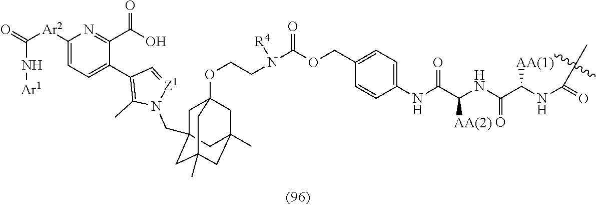

Scheme 14 describes the installment of alternative mAb-linker attachments to dipeptide synthons. Compound (88), wherein can be reacted with compound (90) in the presence of a base such as, but not limited to, N-ethyl-N-isopropylpropan-2-amine, to provide compound (91). The reaction is typically performed at ambient temperature in a solvent such as but not limited to N,N-dimethylformamide. Compound (92) can be prepared by reacting compound (91) with diethylamine. The reaction is typically performed at ambient temperature in a solvent such as but not limited to N,N-dimethylformamide. Compound (93), wherein X¹ is Cl, Br, or I, can be reacted with compound (92), under amidation conditions described herein or readily available in the literature to provide compound (94). Compound (92) can be reacted with compounds of formula (95) under amidation conditions described herein or readily available in the literature to provide compound (96).

### 4.9.23. Synthesis of Compound (106)

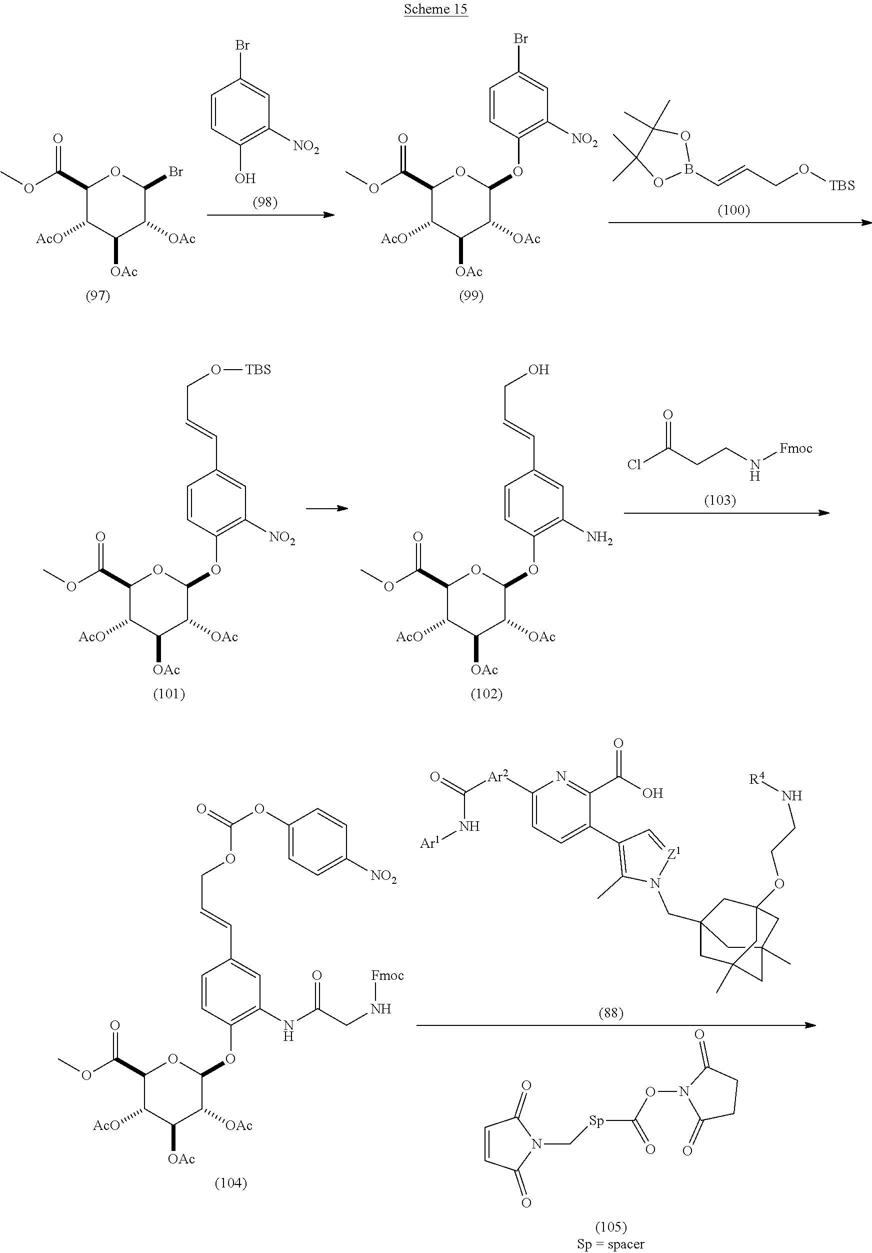

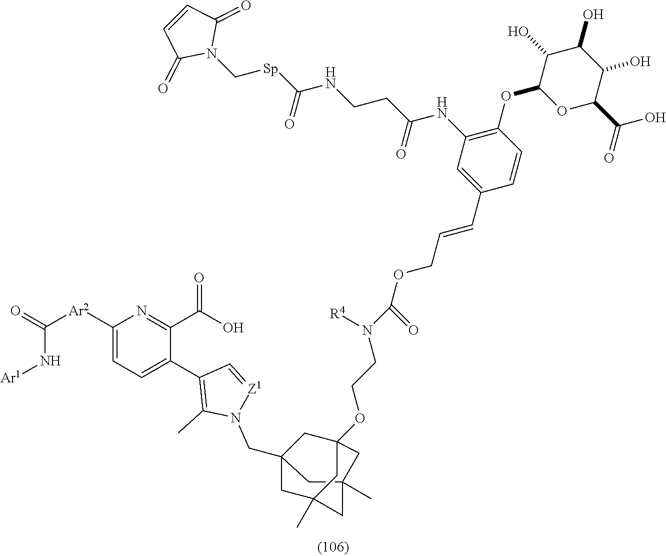

Scheme 15 describes the synthesis of vinyl glucuronide linker intermediates and synthons. (2R,3R,4S,5S,6S)-2-Bromo-6-(methoxycarbonyl)tetrahydro-2H-pyran-3,4,5-triyl triacetate (97) can be treated with silver oxide, followed by 4-bromo-2-nitrophenol (98) to provide (2S,3R,4S,5S,6S)-2-(4-bromo-2-nitrophenoxy)-6-(methoxycarbonyl)tetrahydro-2H-pyran-3,4,5-triyl triacetate (99). The reaction is typically performed at ambient temperature in a solvent, such as, but not limited to, acetonitrile. (2S,3R,4S,5S,6S)-2-(4-Bromo-2-nitrophenoxy)-6-(methoxycarbonyl)tetrahydro-2H-pyran-3,4,5-triyl triacetate (99) can be reacted with (E)-tert-butyldimethyl((3-(4,4,5,5-tetramethyl-1,3,2-dioxaborolan-2-yl)allyl)oxy)silane (100) in the presence of a base such as, but not limited to, sodium carbonate, and a catalyst such as but not limited to tris(dibenzylideneacetone)dipalladium (Pd2(dba)3), to provide (2S,3R,4S,5S,6S)-2-(4-((E)-3-((tert-butyldimethylsilyl)ox)prop-1-en-1-yl)-2-nitrophenoxy)-6-(methoxycarbonyl)tetrahydro-2H-pyran-3,4,5-triyl triacetate (101). The reaction is typically performed at an elevated temperature in a solvent, such as, but not limited to, tetrahydrofuran. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate (102) can be prepared by reacting (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate (101) with zinc in the presence of an acid such as, but not limited to, hydrochloric acid. The addition is typically performed at low temperature before warming to ambient temperature in a solvent such as, but not limited to, tetrahydrofuran, water, or mixtures thereof. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate (102) can be reacted with (9H-fluoren-9-yl)methyl (3-chloro-3-oxopropyl)carbamate (103), in the presence of a base such as, but not limited to, N,N-diisopropylethylamine, to provide (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate (104). The addition is typically performed at low temperature before warming to ambient temperature in a solvent such as, but not limited to dichloromethane. Compound (88) can be reacted with (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate (104) in the presence of a base such as, but not limited to, N-ethyl-N-isopropylpropan-2-amine, followed by work up and reaction with compound (105) in the presence of a base such as, but not limited to, N,N-diisopropylethylamine to provide compound (106). The reactions are typically performed at ambient temperature in a solvent such as, but not limited to N,N-dimethylformamide.

### 4.9.2.4. Synthesis of Compound (115)

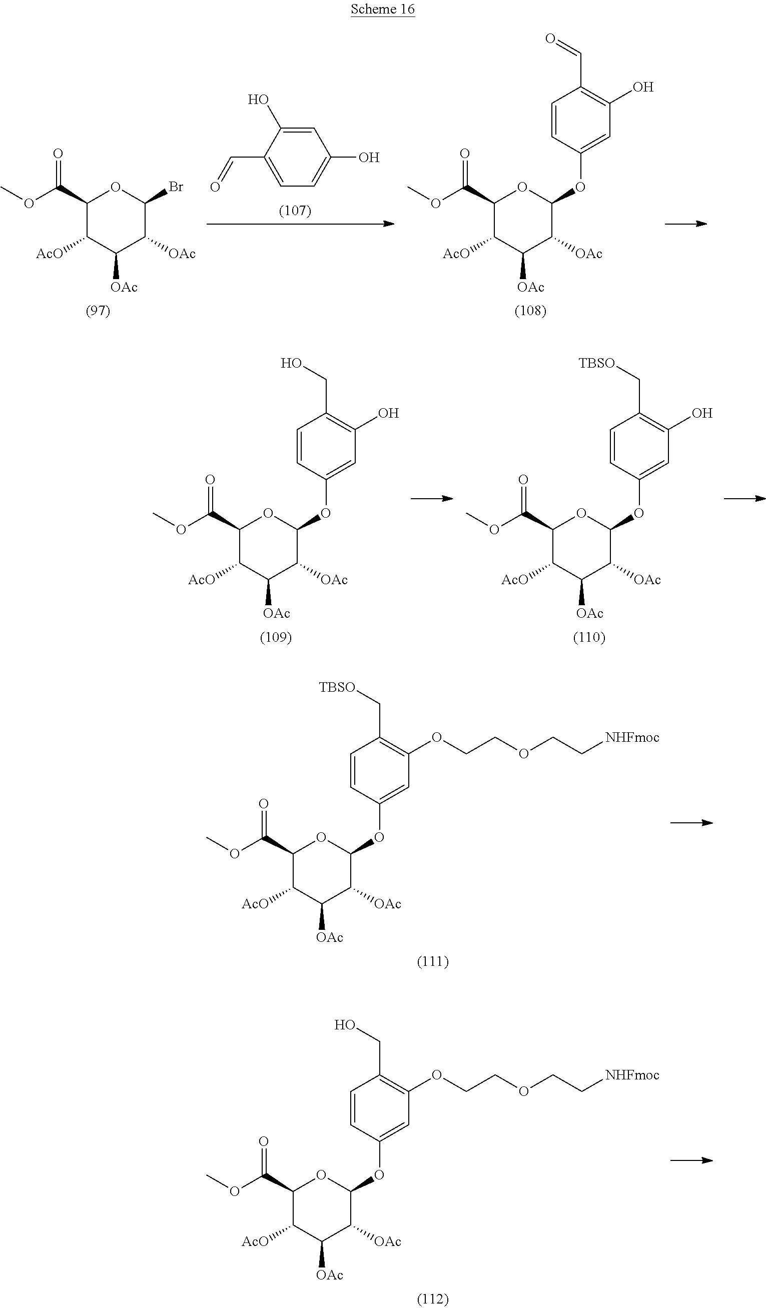

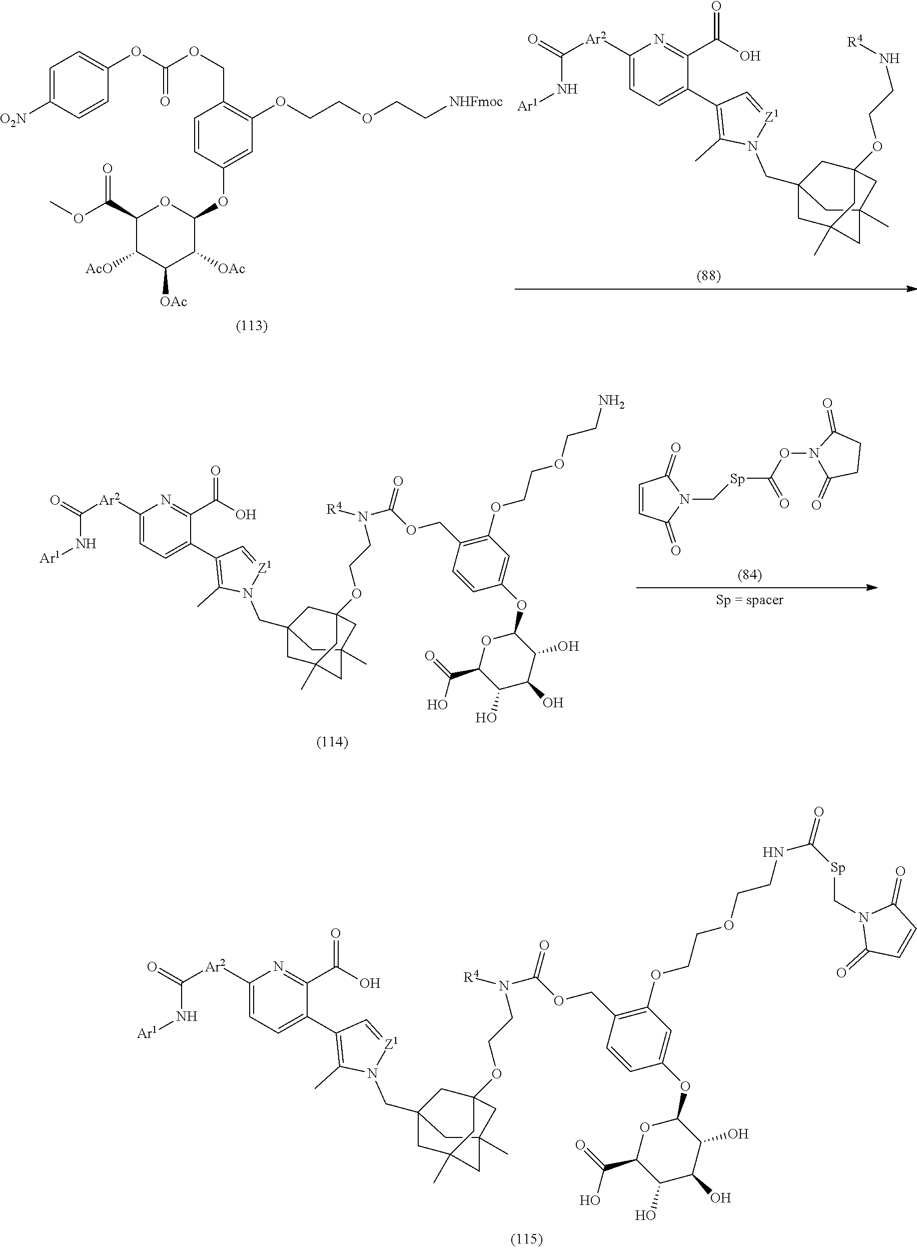

Scheme 16 describes the synthesis of a representative 2-ether glucuronide linker intermediate and synthon. (2S,3R,4S,5S,6S)-2-Bromo-6-(methoxycarbonyl)tetrahydro-2H-pyran-3,4,5-triyl triacetate (97) can be reacted with 2,4-dihydroxybenzaldehyde (107) in the presence of silver carbonate to provide (2S,3R,4S,5S,6S)-2-(4-formyl-3-hydroxyphenoxy)-6-(methoxycarbonyl)tetrahydr-2H-pyran-3,4,5-triyl triacetate (108). The reaction is typically performed at an elevated temperature in a solvent, such as, but not limited to, acetonitrile. (2S,3R4S,5S,6S)-2-(4-Formyl-3-hydroxyphenoxy)-6-(methoxycarbonyl)tetrahydro-2H-pyran-3,4,5-triyl triacetate (108) can be treated with sodium borohydride to provide (2S,3R,4S,5S,6S)-2-(3-hydroxy-4-(hydroxymethyl)phenoxy)-6-(methoxycarbonyl)tetrahydro-2H-pyran-3,4,5-triyl triacetate (109). The addition is typically performed at low temperature before warming to ambient temperature in a solvent such as but not limited to tetrahydrofuran, methanol, or mixtures thereof. (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate (110) can be prepared by reacting (2S,3R,4S,5S,6S)-2-(3-hydroxy-4-(hydroxymethyl)phenoxy)-6-(methoxycarbonyl)tetrahydro-2H-pyran-3,4,5-triyl triacetate (109) with tert-butyldimethylsilyl chloride in the presence of imidazole. The reaction is typically performed at low temperature in a solvent, such as, but not limited to, dichloromethane. (2S,3R,4S,5S,6S)-2-(3-(2-(2-((((9H-Fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate (111) can be prepared by reacting (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate (110) with (9H-fluoren-9-yl)methyl (2-(2-hydroxyethoxy)ethyl)carbamate in the presence of triphenylphosphine and a azodicarboxylate such as, but not limited to, di-tert-butyl diazene-1,2-dicarboxylate. The reaction is typically performed at ambient temperature in a solvent such as but not limited to toluene. (2S,3R,4S,5S,6S)-2-(3-(2-(2-((((9H-Fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate (111) can be treated with acetic acid to provide (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate (112). The reaction is typically performed at ambient temperature in a solvent such as but not limited to water, tetrahydrofuran, or mixtures thereof. (2S,3R,4S,5S,6S)-2-(3-(2-(2-((((9H-Fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate (113) can be prepared by reacting (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate (91) with bis(4-nitrophenyl) carbonate in the presence of a base such as but not limited to N-ethyl-N-isopropylpropan-2-amine. The reaction is typically performed at ambient temperature in a solvent such as but not limited to N,N-dimethylformamide. (2S,3R,4S,5S,6S)-2-(3-(2-(2-((((9H-Fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate (113) can be treated with compound (88) in the presence of a base such as but not limited to N-ethyl-N-isopropylpropan-2-amine, followed by treatment with lithium hydroxide to provide a compound (114). The reaction is typically performed at ambient temperature in a solvent such as but not limited to N,N-dimethylformamide, tetrahydrofuran, methanol, or mixtures thereof. Compound (115) can be prepared by reacting compound (114) with compound (84) in the presence of a base such as but not limited to N-ethyl-N-isopropylpropan-2-amine. The reaction is typically performed at ambient temperature in a solvent such as but not limited to N,N-dimethylformamide.

### 4.9.2.5. Synthesis of Compound (119)

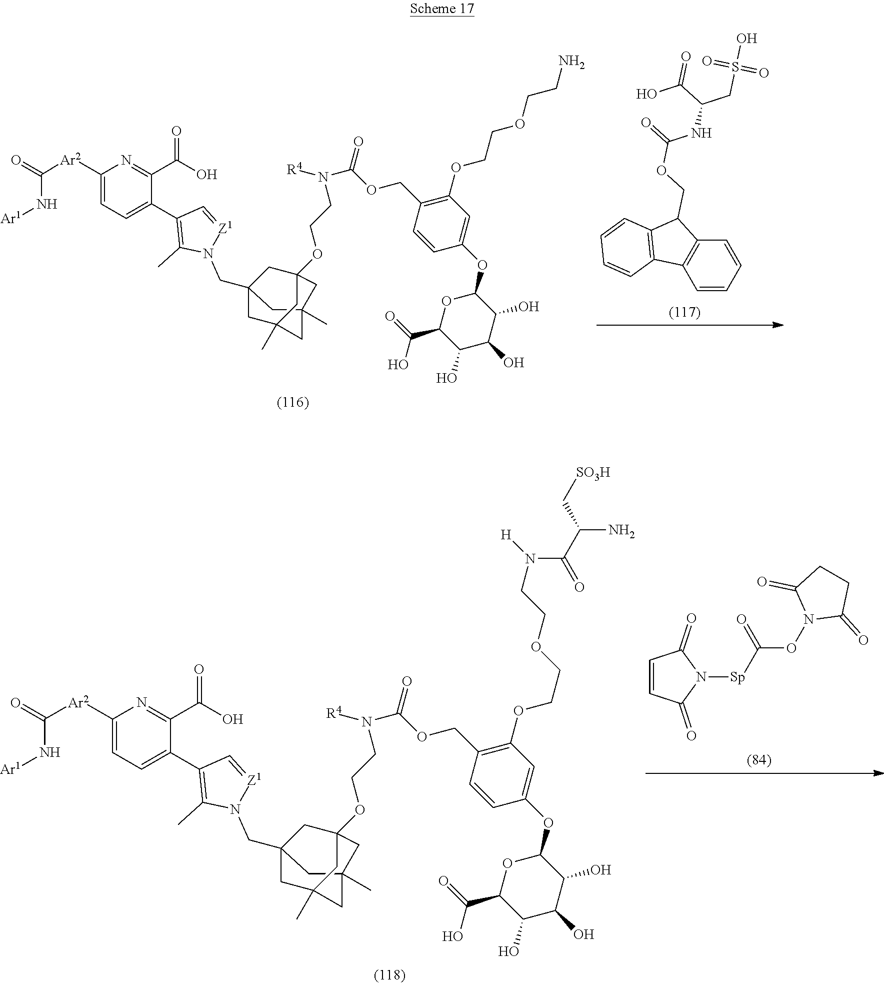

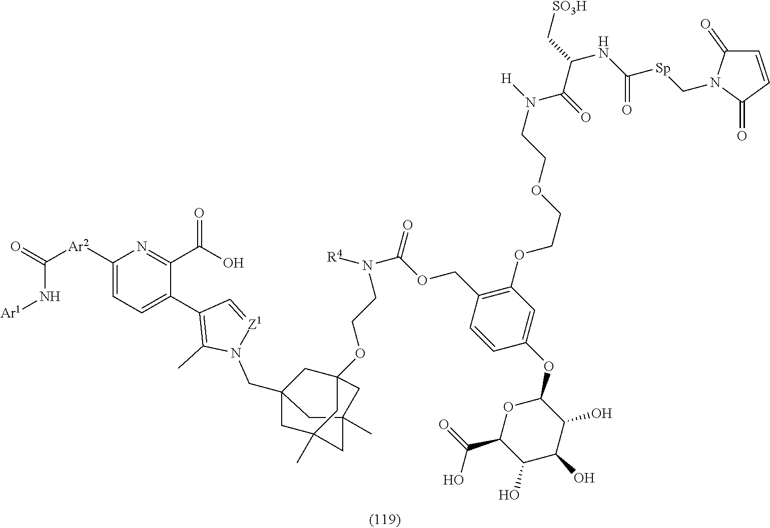

Scheme 17 describes the introduction of a second solubilizing group to a sugar linker. Compound (116) can be reacted with (R)-2-((((9H-fluoren-9-yl)methoxy)carbonyl)amino)-3-sulfopropanoic acid (117), under amidation conditions described herein or readily available in the literature, followed by treatment with a base such as but not limited to diethylamine, to provide compound (118). Compound (118) can be reacted with compound (84), wherein Sp is a spacer, under amidation conditions described herein or readily available in the literature, to provide compound (119).

### 4.9.2.6. Synthesis of Compound (129)

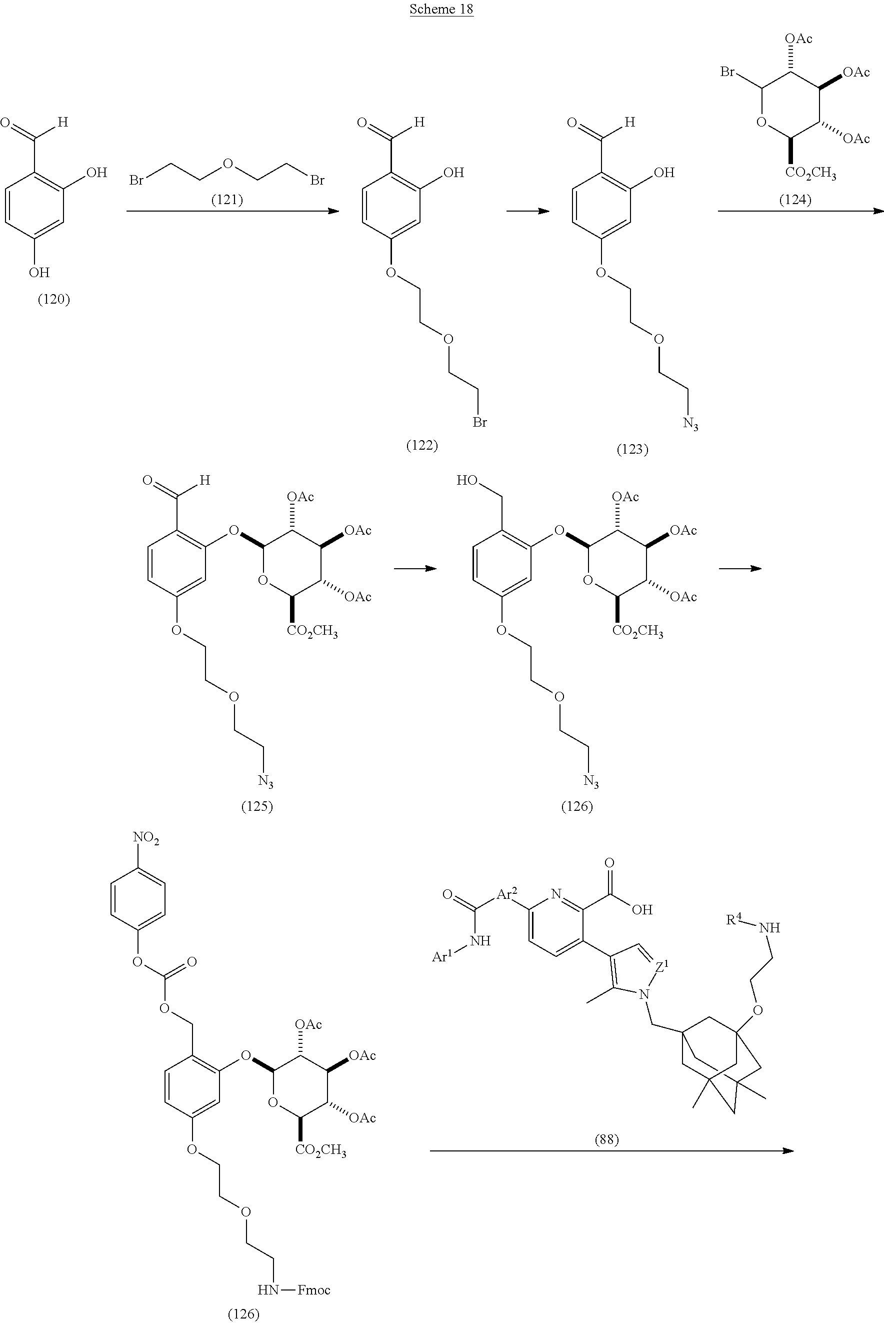

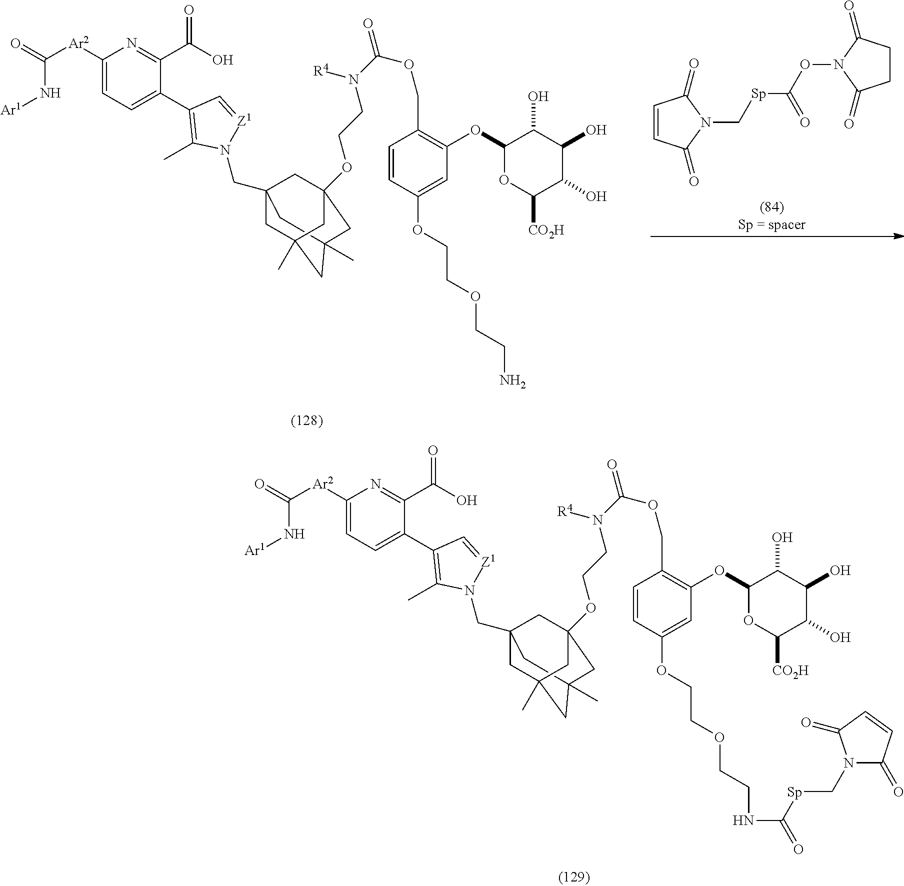

Scheme 18 describes the synthesis of 4-ether glucuronide linker intermediates and synthons. 4-(2-(2-Bromoethoxy)ethoxy)-2-hydroxybenzaldehyde (122) can be prepared by reacting 2,4-dihydroxybenzaldehyde (120) with 1-bromo-2-(2-bromoethoxy)ethane (121) in the presence of a base such as, but not limited to, potassium carbonate. The reaction is typically performed at an elevated temperature in a solvent such as but not limited to acetonitrile. 4-(2-(2-Bromoethoxy)ethoxy)-2-hydroxybenzaldehyde (122) can be treated with sodium azide to provide 4-(2-(2-azidoethoxy)ethoxy)-2-hydroxybenzaldehyde (123). The reaction is typically performed at ambient temperature in a solvent such as but not limited to N,N-dimethylformamide. (2S,3R,4S,5S,6S)-2-(5-(2-(2-Azidoethoxy)ethoxy)-2-formylphenoxy)-6-(methoxycarbonyl)tetrahydro-2H-pyran-3,4,5-triyl triacetate (125) can be prepared by reacting 4-(2-(2-azidoethoxy)ethoxy)-2-hydroxybenzaldehyde (123) with (3R,4S,5S,6S)-2-bromo-6-(methoxycarbonyl)tetrahydro-2H-pyran-3,4,5-triyl triacetate (124) in the presence of silver oxide. The reaction is typically performed at ambient temperature in a solvent such as, but not limited to, acetonitrile. Hydrogenation of (2S,3R,4S,5S,6S)-2-(5-(2-(2-azidoethoxy)ethoxy)-2-formylphenoxy)-6-(methoxycarbonyl)tetrahydro-2H-pyran-3,4,5-triyl triacetate (125) in the presence of Pd/C will provide (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate (126). The reaction is typically performed at ambient temperature in a solvent such as, but not limited to, tetrahydrofuran. (2S,3R,4S,5S,6S)-2-(5-(2-(2-((((9H-Fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate (127) can be prepared by treating (2S,3R4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate (126) with (9H-fluoren-9-yl)methyl carbonochloridate in the presence of a base, such as, but not limited to, N-ethyl-N-isopropylpropan-2-amine. The reaction is typically performed at low temperature in a solvent such as, but not limited to, dichloromethane. Compound (88) can be reacted with (2S,3R4S,5S,6S)-2-(5-(2-(2-((((9H-Fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate (127) in the presence of a base, such as, but not limited to, N-ethyl-N-isopropylpropan-2-amine, followed by treatment with lithium hydroxide to provide compound (128). The reaction is typically performed at low temperature in a solvent such as, but not limited to, N,N-dimethylformamide. Compound (129) can be prepared by reacting compound (128) with compound (84) in the presence of a base such as, but not limited to, N-ethyl-N-isopropylpropan-2-amine. The reaction is typically performed at ambient temperature in a solvent such as but not limited to N,N-dimethylformamide.

### 4.9.2.7. Synthesis of Compound (139)

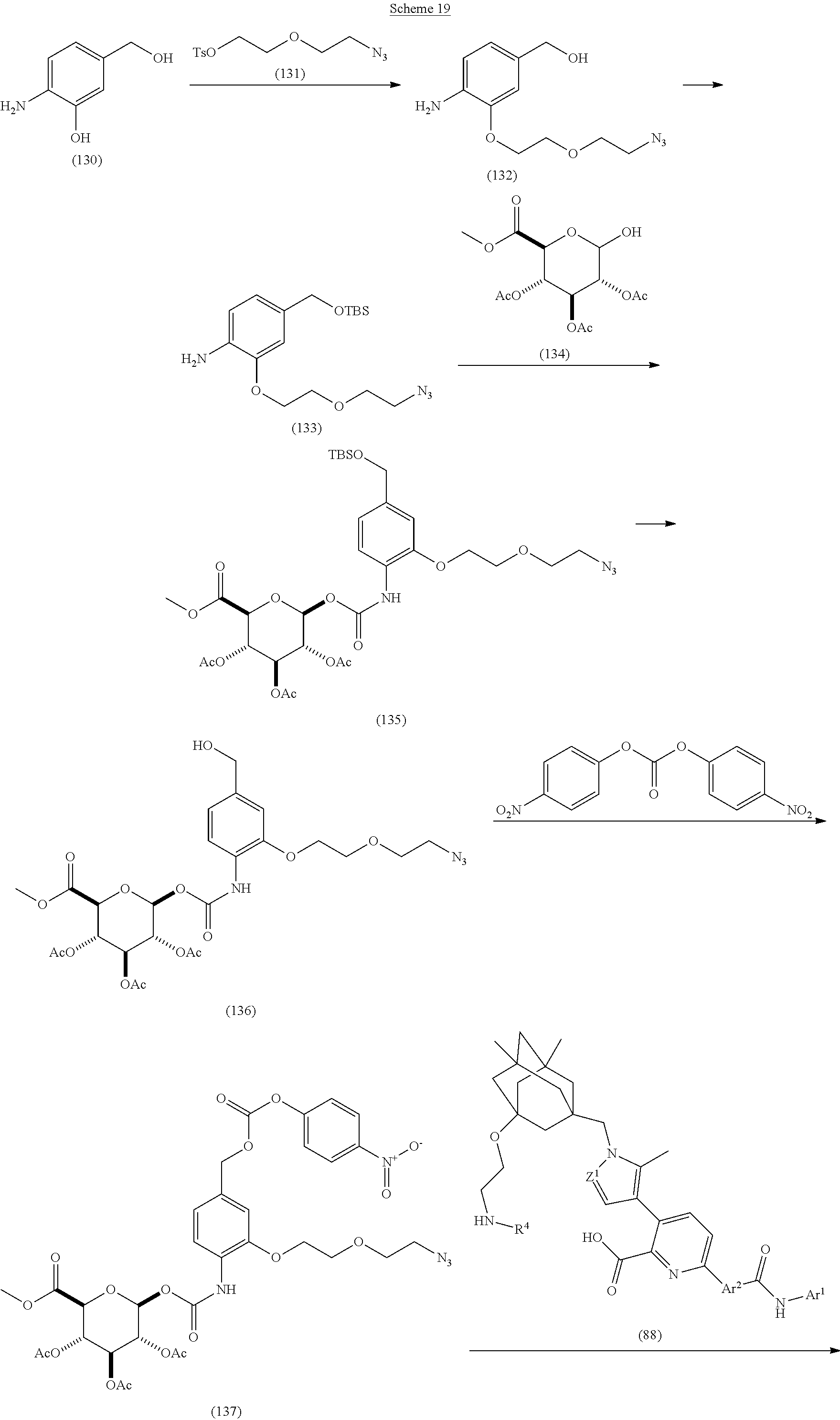

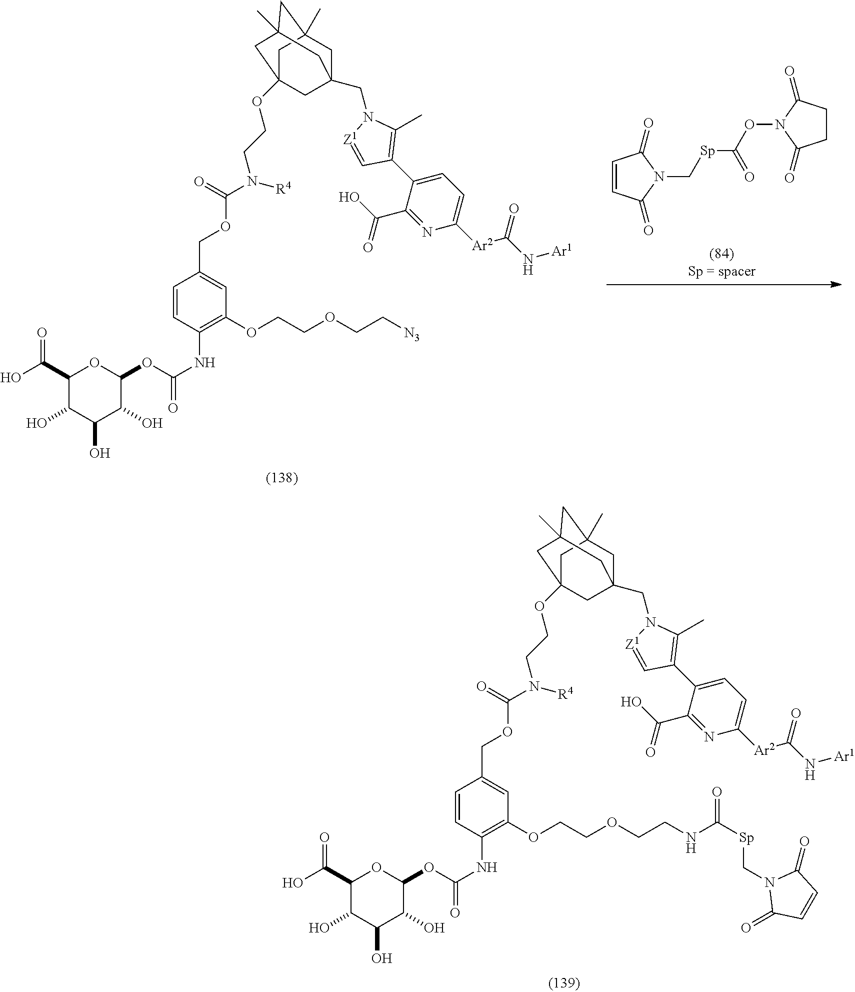

Scheme 19 describes the synthesis of carbamate glucuronide intermediates and synthons. 2-Amino-5-(hydroxymethyl)phenol (130) can be treated with sodium hydride and then reacted with 2-(2-azidoethoxy)ethyl 4-methylbenzenesulfonate (131) to provide (4-amino-3-(2-(2-azidoethoxy)ethoxy)phenyl)methanol (132). The reaction is typically performed at an elevated temperature in a solvent such as, but not limited to N,N-dimethylformamide. 2-(2-(2-Azidoethoxy)ethoxy)-4-(((tert-butyldimethylsilyl)oxy)methyl)aniline (133) can be prepared by reacting (4-amino-3-(2-(2-azidoethoxy)ethoxy)phenyl)methanol (132) with tert-butyldimethylchlorosilane in the presence of imidazole. The reaction is typically performed at ambient temperature in a solvent such as, but not limited to tetrahydrofuran. 2-(2-(2-Azidoethoxy)ethoxy)-4-(((tert-butyldimethylsilyl)oxy)methyl)aniline (133) can be treated with phosgene, in the presence of a base such as but not limited to triethylamine, followed by reaction with (3R,4S,5S,6S)-2-hydroxy-6-(methoxycarbonyl)tetrahydro-2H-pyran-3,4,5-triyl triacetate (134) in the presence of a base such as but not limited to triethylamine, to provide 2S,3R,4S,5S,6S)-2-(((2-(2-(2-azidoethoxy)ethoxy)-4-(((tert-butyldimethylsilyl)oxy)methyl)phenyl)carbamoyl)oxy)-6-(methoxycarbonyl)tetrahydro-2H-pyran-3,4,5-triyl triacetate (135). The reaction is typically performed in a solvent such as, but not limited to, toluene, and the additions are typically performed at low temperature, before warming up to ambient temperature after the phosgene addition and heating at an elevated temperature after the (3R,4S,5S,6S)-2-hydroxy-6-(methoxycarbonyl)tetrahydro-2H-pyran-3,4,5-triyl triacetate (134) addition. (2S,3R,4S,5S,6S)-2-(((2-(2-(2-Azidoethoxy)ethoxy)-4-(hydroxymethyl)phenyl)carbamoyl)oxy)-6-(methoxycarbonyl)tetrahydro-2H-pyran-3,4,5-triyl triacetate (136) can be prepared by reacting 2S,3R,4S,5S,6S)-2-(((2-(2-(2-azidoethoxy)ethoxy)-4-(((tert-butyldimethylsilyl)oxy)methyl)phenyl)carbamoyl)oxy)-6-(methoxycarbonyl)tetrahydro-2H-pyran-3,4,5-triyl triacetate (135) with p-toluenesulfonic acid monohydrate. The reaction is typically performed at ambient temperature in a solvent such as, but not limited to methanol. (2S,3R,4S,5S,6S)-2-(((2-(2-(2-Azidoethoxy)ethoxy)-4-(hydroxymethyl)phenyl)carbamoyl)oxy)-6-(methoxycarbonyl)tetrahydro-2H-pyran-3,4,5-triyl triacetate (136) can be reacted with bis(4-nitrophenyl)carbonate in the presence of a base such as, but not limited to, N,N-diisopropylethylamine, to provide (2S,3R,4S,5S,6S)-2-(((2-(2-(2-azidoethoxy)ethoxy)-4-((((4-nitrophenoxy)carbonyl)oxy)methyl)phenyl)carbamoyl)oxy)-6-(methoxycarbonyl)tetrahydro-2H-pyran-3,4,5-triyl triacetate (137). The reaction is typically performed at ambient temperature in a solvent such as, but not limited to, N,N-dimethylformamide. (2S,3R,4S,5S,6S)-2-(((2-(2-(2-Azidoethoxy)ethoxy)-4-((((4-nitrophenoxy)carbonyl)oxy)methyl)phenyl)carbamoyl)oxy)-6-(methoxycarbonyl)tetrahydro-2H-pyran-3,4,5-triyl triacetate (137) can be reacted with compound in the presence of a base such as, but not limited to, N,N-diisopropylethylamine, followed by treatment with aqueous lithium hydroxide, to provide compound (138). The first step is typically conducted at ambient temperature in a solvent such as, but not limited to N,N-dimethylformamide, and the second step is typically conducted at low temperature in a solvent such as but not limited to methanol. Compound (138) can be treated with tris(2-carboxyethyl))phosphine hydrochloride, followed by reaction with compound (84) in the presence of a base such as, but not limited to, N,N-diisopropylethylamine, to provide compound (139). The reaction with tris(2-carboxyethyl))phosphine hydrochloride is typically performed at ambient temperature in a solvent such as, but not limited to, tetrahydrofuran, water, or mixtures thereof, and the reaction with N-succinimidyl 6-maleimidohexanoate is typically performed at ambient temperature in a solvent such as, but not limited to, N,N-dimethylformamide.

### 4.9.2.8. Synthesis of Compound (149)

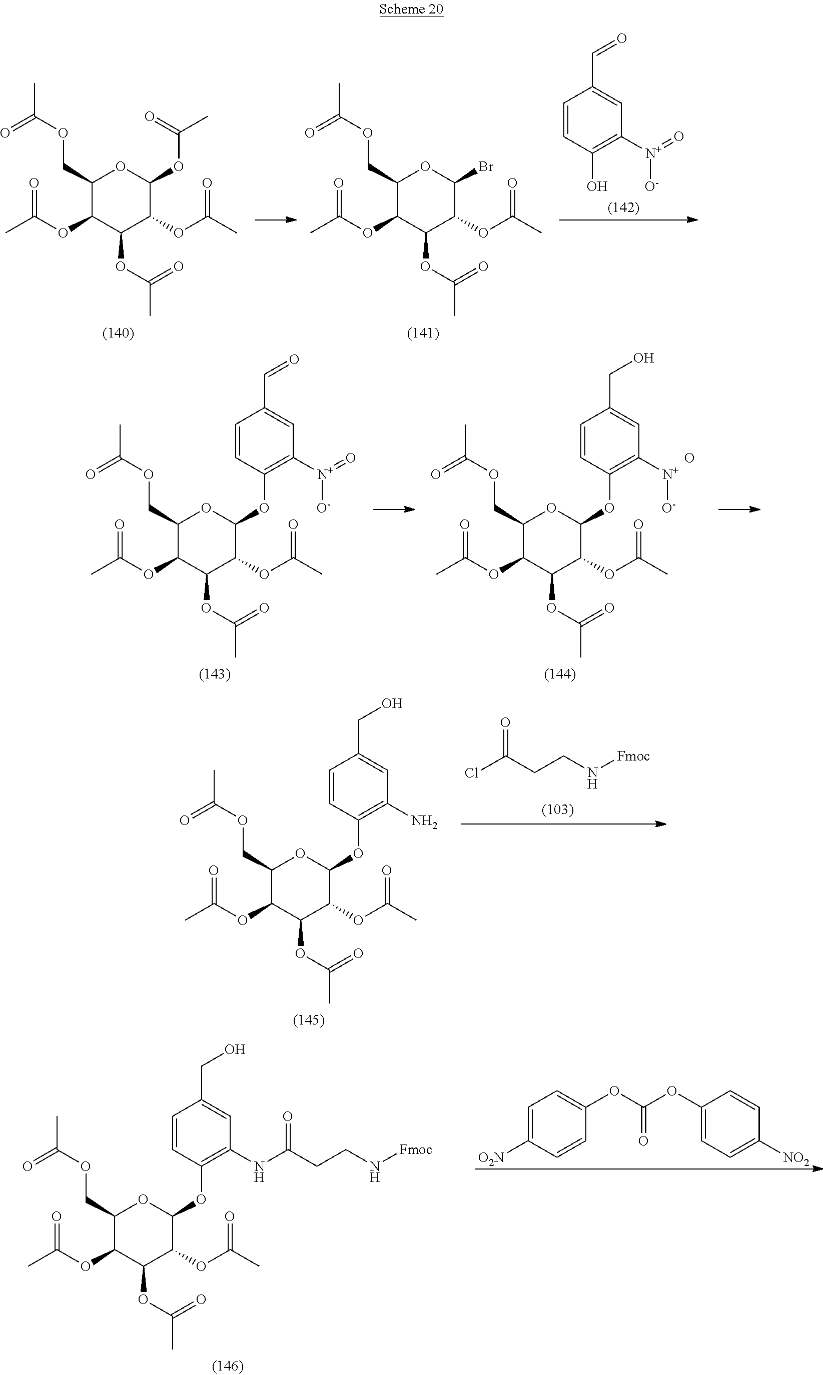

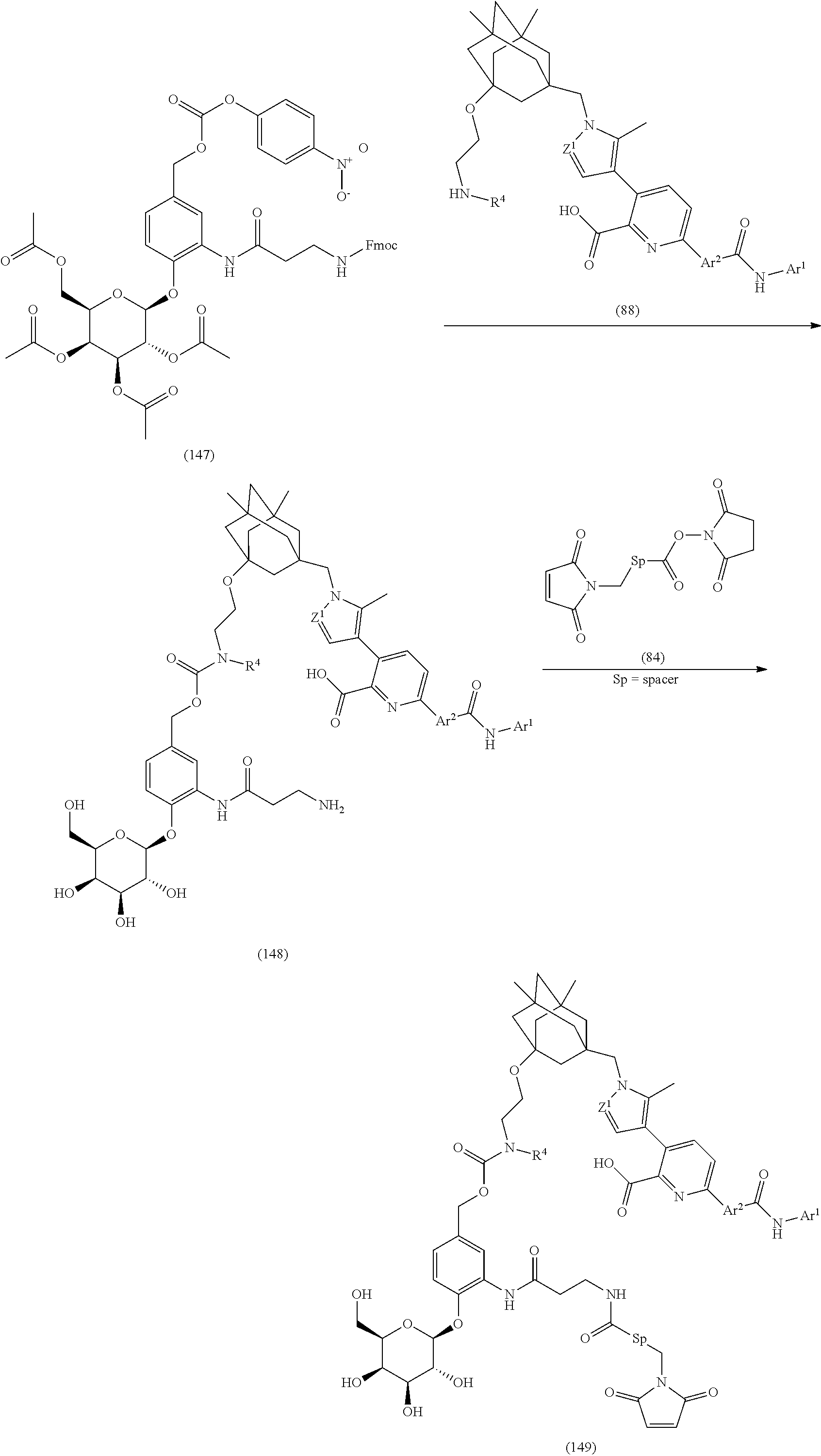

Scheme 20 describes the synthesis of galactoside linker intermediates and synthons. (2S,3R,4S,5S,6R)-6-(Acetoxmethyl)tetrahydro-2H-pyran-2,3,4,5-tetrayl tetraacetate (140) can be treated with HBr in acetic acid to provide (2R,3S,4S,5R,6S)-2-(acetoxymethyl)-6-bromotetrahydro-2H-pyran-3,4,5-triyl triacetate (141). The reaction is typically performed at ambient temperature under a nitrogen atmosphere. (2R,3S,4S,5R,6S)-2-(Acetoxymethyl)-6-(4-formyl-2-nitrophenoxy)tetrahydro-2H-pyran-3,4,5-triyl triacetate (143) can be prepared by treating (2R,3S,4S,5R,6S)-2-(acetoxymethyl)-6-bromotetrahydro-2H-pyran-3,4,5-triyl triacetate (141) with silver(I) oxide in the presence of 4-hydroxy-3-nitrobenzaldehyde (142). The reaction is typically performed at ambient temperature in a solvent such as, but not limited to, acetonitrile. (2R,3S,4S,5R,6S)-2-(Acetoxymethyl)-6-(4-formyl-2-nitrophenoxy)tetrahydro-2H-pyran-3,4,5-triyl triacetate (143) can be treated with sodium borohydride to provide (2R,3S,4S,5R,6S)-2-(acetoxymethyl)-6-(4-(hydroxymethyl)-2-nitrophenoxy)tetrahydro-2H-pyran-3,4,5-triyl triacetate (144). The reaction is typically performed at low temperature in a solvent such as but not limited to tetrahydrofuran, methanol, or mixtures thereof. (2R,3S,4S,5R,6S)-2-(Acetoxymethyl)-6-(2-amino-4-(hydroxymethyl)phenoxy)tetrahydro-2H-pyran-3,4,5-triyl triacetate (145) can be prepared by treating (2R,3S,4S,5R6S)-2-(acetoxymethyl)-6-(4-(hydroxymethyl)-2-nitrophenoxy)tetrahydro-2H-pyran-3,4,5-triyl triacetate (144) with zinc in the presence of hydrochloric acid. The reaction is typically performed at low temperature, under a nitrogen atmosphere, in a solvent such as, but not limited to, tetrahydrofuran. (2S,3R,4S,5S,6R)-2-(2-(3-((((9H-Fluoren-9-yl)methoxy)carbonyl)amino)propanamido)-4-(hydroxymethyl)phenoxy)-6-(acetoxymethyl)tetrahydro-2H-pyran-3,4,5-triyl triacetate (146) can be prepared by reacting (2R,3S,4S,5R,6S)-2-(acetoxymethyl)-6-(2-amino-4-(hydroxymethyl)phenoxy)tetrahydro-2H-pyran-3,4,5-triyl triacetate (145) with (9H-fluoren-9-yl)methyl (3-chloro-3-oxopropyl)carbamate (103) in the presence of a base such as, but not limited to, N,N-diisopropylethylamine. The reaction is typically performed at low temperature, in a solvent such as, but not limited to, dichloromethane. (2S,3R,4S,5S,6R)-2-(2-(3-((((9H-Fluoren-9-yl)methoxy)carbonyl)amino)propanamido)-4-(hydroxymethyl)phenoxy)-6-(acetoxymethyl)tetrahydro-2H-pyran-3,4,5-triyl triacetate (146) can be reacted with bis(4-nitrophenyl)carbonate in the presence of a base such as, but not limited to, N,N-diisopropylethylamine, to provide (2S,3R,4S,5S,6R)-2-(2-(3-((((9H-fluoren-9-yl)methoxy)carbonyl)amino)propanamido)-4-((((4-nitrophenoxy)carbonyl)oxy)methyl)phenoxy)-6-(acetoxymethyl)tetrahydro-2H-pyran-3,4,5-triyl triacetate (147). The reaction is typically performed at low temperature, in a solvent such as, but not limited to, N,N-dimethylformamide. (2S,3R,4S,5S,6R)-2-(2-(3-((((9H-Fluoren-9-yl)methoxy)carbonyl)amino)propanamido)-4-((((4-nitrophenoxy)carbonyl)oxy)methyl)phenoxy)-6-(acetoxymethyl)tetrahydro-2H-pyran-3,4,5-triyl triacetate (147) can be reacted with compound (88) in the presence of a base such as, but not limited to N,N-diisopropylethylamine, followed by treatment with lithium hydroxide, to provide compound (148). The first step is typically performed at low temperature, in a solvent such as, but not limited to, N,N-dimethylformamide, and the second step is typically performed at ambient temperature, in a solvent such as, but not limited to, methanol. Compound (148) can be treated with compound (84), wherein Sp is a spacer, in the presence of a base, such as, but not limited to N,N-diisopropylethylamine, to provide compound (149). The reaction is typically performed at ambient temperature, in a solvent such as, but not limited to, N,N-dimethylformamide.

### 4.10. Compositions

The Bcl-xL inhibitors and/or ADCs described herein may be in the form of compositions comprising the inhibitor or ADC and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the inhibitors and/or ADCs and, for therapeutic uses, the mode of administration.

For therapeutic uses, the Bcl-xL inhibitor and/or ADC compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intrathecally, topically or locally. The most suitable route for administration in any given case will depend on the particular Bcl-xL inhibitor or ADC, the subject, and the nature and severity of the disease and the physical condition of the subject. Typically, the Bcl-xL inhibitors will be administered orally or parenterally, and ADC pharmaceutical composition will be administered intravenously or subcutaneously.

Pharmaceutical compositions can be conveniently presented in unit dosage forms containing a predetermined amount of Bcl-xL inhibitor or an ADC described herein per dose. The quantity of inhibitor or ADC included in a unit dose will depend on the disease being treated, as well as other factors as are well known in the art. For Bcl-xL inhibitors, such unit dosages may be in the form of tablets, capsules, lozenges, etc. containing an amount of Bcl-xL inhibitor suitable for a single administration. For ADCs, such unit dosages may be in the form of a lyophilized dry powder containing an amount of ADC suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of ADC suitable for a single administration.

The pharmaceutical compositions may also be supplied in bulk from containing quantities of ADC suitable for multiple administrations.

Pharmaceutical compositions of ADCs may be prepared for storage as lyophilized formulations or aqueous solutions by mixing an ADC having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See. Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at concentration ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), Pluronic polyols, polyoxyethylene sorbitan monoethers (TWEEN®-20, TWEEN®-80, etc.). Non-ionic surfactants may be present in a range of about 0.05 mg/ml to about 1.0 mg/ml, for example about 0.07 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

### 4.11. Methods of Use

The Bcl-xL inhibitors included in the ADCs, as well as the synthons delivered by the ADCs, inhibit Bcl-xL activity and induce apoptosis in cells expressing Bcl-xL. Accordingly, the Bcl-xL inhibitors and/or ADCs may be used in methods to inhibit Bcl-xL activity and/or induce apoptosis in cells.

For Bcl-xL inhibitors, the method generally involves contacting a cell whose survival depends, at least in part, upon Bcl-xL expression with an amount of a Bcl-xL inhibitor sufficient to inhibit Bcl-xL activity and/or induce apoptosis. For ADCs, the method generally involves contacting a cell whose survival depends, at least in part upon Bcl-xL expression, and that expresses a cell-surface antigen for the antibody of the ADC with an ADC under conditions in which the ADC binds the antigen.

In certain embodiments, the antibody of the ADC binds a target capable of internalizing the ADC into the cell, where it can deliver its Bcl-xL inhibitory synthon. The method may be carried out in vitro in a cellular assay to inhibit Bcl-xL activity and/or inhibit apoptosis, or in vivo as a therapeutic approach towards treating diseases in which inhibition of apoptosis and/or induction of apoptosis would be desirable.

Dysregulated apoptosis has been implicated in a variety of diseases, including, for example, autoimmune disorders (e.g., systemic lupus erythematosus, rheumatoid arthritis, graft-versus-host disease, myasthenia gravis, or Sjogren's syndrome), chronic inflammatory conditions (e.g., psoriasis, asthma or Crohn's disease), hyperproliferative disorders (e.g., breast cancer, lung cancer), viral infections (e.g., herpes, papilloma, or HIV), and other conditions, such as osteoarthritis and atherosclerosis. The Bcl-xL inhibitor or ADCs described herein may be used to treat or ameliorate any of these diseases. Such treatments generally involve administering to a subject suffering from the disease an amount of a Bcl-xL inhibitor or ADC described herein sufficient to provide therapeutic benefit. For ADCs, identity of the antibody of the ADC administered will depend upon the disease being treated—to the antibody should bind a cell-surface antigen expressed in the cell type where inhibition of Bcl-xL activity would be beneficial. The therapeutic benefit achieved will also depend upon the specific disease being treated. In certain instances, the Bcl-xL inhibitor or ADC may treat or ameliorate the disease itself, or symptoms of the disease, when administered as monotherapy. In other instances, the Bcl-xL inhibitor or ADC may be part of an overall treatment regimen including other agents that, together with the inhibitor or ADC, treat or ameliorate the disease being treated, or symptoms of the disease. Agents useful to treat or ameliorate specific diseases that may be administered adjunctive to, or with, the Bcl-xL inhibitors and/or ADCs described herein will be apparent to those of skill in the art.

Although absolute cure is always desirable in any therapeutic regimen, achieving a cure is not required to provide therapeutic benefit. Therapeutic benefit may include halting or slowing the progression of the disease, regressing the disease without curing, and/or ameliorating or slowing the progression of symptoms of the disease. Prolonged survival as compared to statistical averages and/or improved quality of life may also be considered therapeutic benefit.

One particular class of diseases that involve dysregulated apoptosis and that are significant health burden world-wide are cancers. In a specific embodiment, the Bcl-xL inhibitors and/or ADCs described herein may be used to treat cancers. The cancer may be, for example, solid tumors or hematological tumors. Cancers that may be treated with the ADCs described herein include, but are not limited to include, but are not limited to bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, myeloma, prostate cancer, small cell lung cancer or spleen cancer. ADCs may be especially beneficial in the treatment of cancers because the antibody can be used to target the Bcl-xL inhibitory synthon specifically to tumor cells, thereby potentially avoiding or ameliorating undesirable side-effects and/or toxicities that may be associated with systemic administration of unconjugated inhibitors. One embodiment pertains to a method of treating a disease involving dysregulated intrinsic apoptosis, comprising administering to a subject having a disease involving dysregulated apotosis an amount of an ADC described herein effective to provide therapeutic benefit, wherein the antibody of the ADC binds a cell surface receptor on a cell whose intrinsic apoptosis is dysregulated. One embodiment pertains to a method of treating cancer, comprising administering to a subject having cancer an ADC described herein that is capable of binding a cell surface receptor or a tumor associated antigen expressed on the surface of the cancer cells, in an amount effective to provide therapeutic benefit.

In the context of tumorigenic cancers, therapeutic benefit, in addition to including the effects discussed above, may also specifically include halting or slowing progression of tumor growth, regressing tumor growth, eradicating one or more tumors and/or increasing patient survival as compared to statistical averages for the type and stage of the cancer being treated. In one embodiment, which the cancer being treated is a tumorigenic cancer.

The Bcl-xL inhibitors and/or ADCs may be administered as monotherapy to provide therapeutic benefit, or may be administered adjunctive to, or with, other chemotherapeutic agents and/or radiation therapy. Chemotherapeutic agents to which the inhibitors and/or ADCs described herein may be utilized as adjunctive therapy may be targeted (for example, other Bcl-xL inhibitors or ADCs, protein kinase inhibitors, etc.) or non-targeted (for example, non-specific cytotoxic agents such as radionucleotides, alkylating agents and intercalating agents). Non-targeted chemotherapeutic agents with which the inhibitors and/or ADCs described herein may be adjunctively administered include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbizine, topotecan, nitrogen mustards, Cytoxan, etoposide, 5-fluorouracil, BCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, calicheamicin, and docetaxel.

Elevated Bcl-xL expression has been shown to correlate with resistance to chemotherapy and radiation therapy (Park et al., 2013. Cancer Res 73:5485-5496). Data herein demonstrate that Bcl-xL inhibitors and/or ADCs that may not be effective as monotherapy to treat cancer may be administered adjunctive to, or with, other chemotherapeutic agents or radiation therapy to provide therapeutic benefit. While not intending to be bound by any therapy of operation, it is believed that administration of the Bcl-xL inhibitors and/or ADCs described herein to tumors that have become resistant to standard of care chemotherapeutic agents and/or radiation therapy sensitizes the tumors such that they again respond to the chemo and/or radiation therapy. Accordingly, in the context of treating cancers, “therapeutic benefit” includes administering the inhibitors and/or ADCs described herein adjunctive to, or with, chemotherapeutic agents and/or radiation therapy, either in patients who have not yet begin such therapy or who have but have not yet exhibited signs of resistance, or in patients who have begun to exhibit signs of resistance, as a means of sensitizing the tumors to the chemo and/or radiation therapy. One embodiment pertains to a method of sensitizing a tumor to standard cytotoxic agents and/or radiation, comprising contacting the tumor with an ADC described herein that is capable of binding the tumor, in an amount effective to sensitize the tumor cell to a standard cytotoxic agent and/or radiation. Another embodiment pertains to a method of sensitizing a tumor to standard cytotoxic agents and/or radiation, comprising contacting the tumor with an ADC described herein that is capable of binding the tumor, in an amount effective to sensitize the tumor cell to a standard cytotoxic agent and/or radiation in which the tumor has become resistant to treatment with standard cytotoxic agents and/or radiation. Another embodiment pertains to a method of sensitizing a tumor to standard cytotoxic agents and/or radiation, comprising contacting the tumor with an ADC described herein that is capable of binding the tumor, in an amount effective to sensitize the tumor cell to a standard cytotoxic agent and/or radiation in which the tumor has not been previously exposed to standard cytotoxic agents and/or radiation therapy.

### 4.12. Dosages and Administration Regimens

The amount of Bcl-xL inhibitor and/or ADC administered will depend upon a variety of factors, including but not limited to, the particular disease being treated, the mode of administration, the desired therapeutic benefit, the stage or severity of the disease, the age, weight and other characteristics of the patient, etc. Determination of effective dosages is within the capabilities of those skilled in the art.

Effective dosages may be estimated initially from cellular assays. For example, an initial dose for use in humans may be formulated to achieve a circulating blood or serum concentration of Bcl-xL inhibitor or ADC that is expected to achieve a cellular concentration of Bcl-xL inhibitor that is at or above an IC₅₀ or ED₅₀ of the particular inhibitory molecule measured in a cellular assay.

Initial dosages for use in humans may also be estimated from in vivo animal models. Suitable animal models for a wide variety of diseases are known in the art.

When administered adjunctive to, or with, other agents, such as other chemotherapeutic agents, the Bcl-xL inhibitors or ADCs may be administered on the same schedule with the other agents, or on a different schedule. When administered on the same schedule, the inhibitor or ADC may be administered before, after, or concurrently with the other agent. In some embodiments where the inhibitor or ADC is administered adjunctive to, or with, standard chemo- and/or radiation therapy, the inhibitor or ADC may be initiated prior to commencement of the standard therapy, for example a day, several days, a week, several weeks, a month, or even several months before commencement of standard chemo- and/or radiation therapy.

When administered adjunctive to, or with, other agents, such as for example standard chemotherapeutic agents, the other agent will typically be administered according to its standard dosing schedule with respect to route, dosage and frequency. However, in some instances less than the standard amount may be necessary for efficacy when administered adjunctive to Bcl-xL inhibitor or ADC therapy.

### 5. EXAMPLES

### Example 1. Synthesis of Exemplary Bcl-xL Inhibitors

This Example provides synthetic methods for exemplary Bcl-xL inhibitory compounds W3.01-W3.42. Bcl-xL inhibitors (W3.01-W3.43) and synthons (Examples 2.1-2.72) were named using ACD/Name 2012 release (Build 56084, 5 Apr. 2012, Advanced Chemistry Development Inc., Toronto, Ontario) or ACD/Name 2014 release (Build 66687, 25 Oct. 2013, Advanced Chemistry Development Inc., Toronto, Ontario). Bcl-xL inhibitor and synthon intermediates were named with ACD/Name 2012 release (Build 56084, 5 Apr. 2012, Advanced Chemistry Development Inc., Toronto, Ontario), ACD/Name 2014 release (Build 66687, 25 Oct. 2013, Advanced Chemistry Development Inc., Toronto, Ontario), ChemDraw® Ver. 9.0.7 (CambridgeSoft Cambridge, Mass.), ChemDraw® Ultra Ver. 12.0 (CambridgeSoft, Cambridge, Mass.), or ChemDraw® Professional Ver. 15.0.0.106.

### 1.1. Synthesis of 6-[1-(1,3-benzothiazol-2-ylcarbamoyl)-1,2,3,4-tetrahydroquinolin-7-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.01)

### 1.1.1. 3-bromo-5,7-dimethyladamantanecarboxylic acid

To a 50 mL round-bottomed flask at 0° C. was added bromine (16 mL). Iron powder (7 g) was added, and the reaction was stirred at 0° C. for 30 minutes. 3,5-Dimethyladamantane-1-carboxylic acid (12 g) was then added. The mixture was then warmed to room temperature and stirred for 3 days. An ice/concentrated HCl mixture was poured into the reaction mixture. The resulting suspension was treated twice with Na₂SO₃(50 g in 200 mL water) and extracted three times with dichloromethane. The combined organic layers were washed with 1N aqueous HCl, dried over Na₂SO₄, filtered, and concentrated to give the crude title compound.

### 1.1.2. 3-bromo-5,7-dimethyladamantanemethanol

To a solution of Example 1.1.1 (15.4 g) in tetrahydrofuran (200 mL) was added BH₃ (1M in tetrahydrofuran, 150 mL). The mixture was stirred at room temperature overnight. The reaction mixture was then carefully quenched via dropwise addition of methanol. The mixture was then concentrated under vacuum and the residue was partitioned between ethyl acetate (500 mL) and 2N aqueous HCl (100 mL). The aqueous layer was further extracted twice with ethyl acetate and the combined organic extracts were combined and washed with water and brine, and dried over Na₂SO₄. Filtration and evaporation of the solvent gave the title compound.

### 1.1.3. 1-((3-bromo-5,7-dimethyltricyclo[3.3.1.1^{3,7}]dec-1-yl)methyl)-1H-pyrazole

To a solution of Example 1.1.2 (8.0 g) in toluene (60 mL) was added 1H-pyrazole (1.55 g) and cyanomethylenetributylphosphorane (2.0 g). The mixture was stirred at 90° C. overnight. The reaction mixture was then concentrated and the residue was purified by silica gel column chromatography (10:1 hexane:ethyl acetate) to provide the title compound. MS (ESI) m/e 324.2 (M+H)⁺.

### 1.1.4. 2-{[3,5-dimethyl-7-(1H-pyrazol-1-ylmethyl)tricyclo[3.3.1.1^{3,7}]dec-1-yl]oxy}ethanol

To a solution of Example 1.1.3 (4.0 g) in ethane-1,2-diol (12 mL) was added triethylamine (3 mL). The mixture was stirred at 150° C. under microwave conditions (Biotage) for 45 minutes. The mixture was poured into water (100 mL) and extracted three times with ethyl acetate. The combined organic extracts were washed with water and brine, and dried over Na₂SO₄. Filtration and evaporation of the solvent gave the crude title compound which was purified via column chromatography, eluting with 20% ethyl acetate in hexane followed by 5% methanol in dichloromethane, to provide the title compound. MS (ESI) m/e 305.2 (M+H)⁺.

### 1.1.5. 2-({3,5-dimethyl-7-[(5-methyl-1H-pyrazol-1-yl)methyl]tricyclo[3.3.1.1^{3,7}]dec-1-yl}oxy)ethanol

To a cooled (−78° C.) solution of Example 1.1.4 (6.05 g) in tetrahydrofuran (100 mL) was added n-BuLi (40 mL, 2.5M in hexane). The mixture was stirred at −78° C. for 1.5 hours. Then, iodomethane (10 mL) was added through a syringe and the mixture was stirred at −78° C. for 3 hours. The reaction mixture was then quenched with aqueous NH₄Cl and extracted twice with ethyl acetate, and the combined organic extracts were washed with water and brine. After drying over Na₂SO₄, the solution was filtered and concentrated and the residue was purified by silica gel column chromatography (5% methanol in dichloromethane) to provide the title compound. MS (ESI) m/e 319.5 (M+H)⁺.

### 1.1.6. 1-({3,5-dimethyl-7-[2-(hydroxy)ethoxy]tricyclo[3.3.1.1^{3,7}]dec-1-yl)}methyl)-4-iodo-5-methyl-1H-pyrazole

To a solution of Example 1.1.5 (3.5 g) in N,N-dimethylformamide (30 mL) was added N-iodosuccinimide (3.2 g). The mixture was stirred at room temperature for 1.5 hours. The reaction mixture was then diluted with ethyl acetate (600 mL) and washed with aqueous NaHSO₃, water, and brine. After drying over Na₂SO₄, the solution was filtered and concentrated and the residue was purified by silica gel chromatography (20% ethyl acetate in dichloromethane) to give the title compound. MS (ESI) m/e 445.3 (M+H)⁺.

### 1.1.7. 2-((3-((4-iodo-5-methyl-1H-pyrazol-1-yl)methyl)-5,7-dimethyladamantan-1-yl)oxy)ethyl methanesulfonate

To a cooled solution (0° C.) of Example 1.1.6 (5.45 g) in dichloromethane (100 mL) was added triethylamine (5.13 mL) and methanesulfonyl chloride (0.956 mL). The mixture was stirred at room temperature for 1.5 hours, diluted with ethyl acetate (600 mL) and washed with water (120 mL) and brine (120 mL). The organic layer was dried over Na₂SO₄, filtered, and concentrated to provide the title compound. MS (ESI) m/e 523.4 (M+H)⁺.

### 1.1.8. 2-((3-((4-iodo-5-methyl-1H-pyrazol-1-yl)methyl)-5,7-dimethyladamantan-1-yl)oxy)-N-methylethanamine

A solution of Example 1.1.7 (6.41 g) in 2M methylamine in ethanol (15 mL) was stirred at overnight and concentrated. The residue was diluted with ethyl acetate and washed with aqueous NaHCO₃, water and brine. The organic layer was dried over Na₂SO₄, filtered, and concentrated to provide the title compound. MS (ESI) m/e 458.4 (M+H)⁺.

### 1.1.9. tert-butyl [2-({3-[(4-iodo-5-methyl-1H-pyrazol-1-yl)methyl]-5,7-dimethyltricyclo[3.3.1.1^{3,7}]dec-1-yl}oxy)ethyl]methylcarbamate

To a solution of Example 1.1.8 (2.2 g) in tetrahydrofuran (30 mL) was added di-tert-butyl dicarbonate (1.26 g) and a catalytic amount of 4-dimethylaminopyridine. The mixture was stirred at room temperature for 1.5 hours and then diluted with ethyl acetate (300 mL). The solution was washed with saturated aqueous NaHCO₃, water (60 mL) and brine (60 mL). The organic layer was dried with Na₂SO₄, filtered and concentrated. The residue was purified by silica gel chromatography, eluting with 20% ethyl acetate in dichloromethane, to provide the title compound. MS (ESI) m/e 558.5 (M+H)⁺.

### 1.1.10. tert-butyl (2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethyl)(methyl)carbamate

To a solution of Example 1.1.9 (1.2 g) in dioxane was added bis(benzonitrile)palladium(II) chloride (0.04 g), 4,4,5,5-tetramethyl-1,3,2-dioxaborolane (0.937 mL) and triethylamine (0.9 mL). The mixture was heated at reflux overnight, diluted with ethyl acetate and washed with water (60 mL) and brine (60 mL). The organic layer was dried over Na₂SO₄, filtered and concentrated to provide the title compound. MS (ESI) m/e 558.5 (M+H)⁺.

### 1.1.11. tert-butyl 3-(1-((3-(2-((tert-butoxycarbonyl)(methyl)amino) ethoxy)-5,7-dimethyladamantan-1-yl)methyl)-5-methyl-1H-pyrazol-4-yl)-6-chloropicolinate

To Example 1.1.10 (100 mg) and tert-butyl 3-bromo-6-chloropicolinate (52.5 mg) in dioxane (2 mL) was added tris(dibenzylideneacetone)dipalladium(0) (8.2 mg), K₃PO₄ (114 mg), 1,3,5,7-tetramethyl-8-phenyl-2,4,6-trioxa-8-phosphaadamantane (5.24 mg) and water (0.8 mL). The mixture was stirred at 95° C. for 4 hours, diluted with ethyl acetate and washed with water and brine. The organic layer was dried over Na₂SO₄, filtered, concentrated and purified by flash chromatography, eluting with 20% ethyl acetate in heptanes and then with 5% methanol in dichloromethane, to provide the title compound. MS (ESI) m/e 643.3 (M+H)⁺.

### 1.1.12. tert-butyl 3-(1-((3-(2-((tert-butoxycarbonyl)(methyl)amino)ethoxy)-5,7-dimethyladamantan-1-yl)methyl)-5-methyl-1H-pyrazol-4-yl)-6-(1,2,3,4-tetrahydroquinolin-7-yl)picolinate

A mixture of Example 1.1.11 (480 mg), 7-(4,4,5,5-tetramethyl-1,3,2-dioxaborolan-2-yl)-1,2,3,4-tetrahydroquinoline (387 mg), dichlorobis(triphenylphosphine)-palladium(1) (78 mg) and CsF (340 mg) in dioxane (12 mL) and water (5 mL) was heated at 100° C. for 5 hours. After this time the reaction mixture was allowed to cool to room temperature and then diluted with ethyl acetate. The resulting mixture was washed with water and brine, and the organic layer was dried over Na₂SO₄, filtered, and concentrated. The residue was purified by flash chromatography, eluting with 50% ethyl acetate in heptanes to provide the title compound. MS (APCI) m/e 740.4 (M+H)⁺.

### 1.1.13. tert-butyl 6-(1-(benzo[d]thiazol-2-ylcarbamoyl)-1,2,3,4-tetrahydroquinolin-7-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a solution of benzo[d]thiazol-2-amine (114 mg) in acetonitrile (5 mL) was added bis(2,5-dioxopyrrolidin-1-yl) carbonate (194 mg). The mixture was stirred for 1 hour, and Example 1.1.12 (432 mg) in acetonitrile (5 mL) was added. The mixture was stirred overnight, diluted with ethyl acetate, washed with water and brine, and the organic layer was dried over Na₂SO₄, filtered, and concentrated. The residue was purified by flash chromatography, eluting with 50% ethyl acetate in heptanes to provide the title compound.

### 1.1.14. 6-(1-(benzo[d]thiazol-2-ylcarbamoyl)-1,2,3,4-tetrahydroquinolin-7-yl)-3-(1-((3,5-dimethyl-7-(2-(methylamino)ethoxy)adamantan-1-yl)methyl)-5-methyl-1H-pyrazol-4-yl)picolinic acid

Example 1.1.13 (200 mg) in dichloromethane (5 mL) was treated with trifluoroacetic acid (2.5 mL) overnight. The mixture was concentrated to provide the title compound. ¹H NMR (400 MHz, dimethyl sulfoxide-d₆) δ ppm 8.40 (s, 1H), 8.30 (s, 2H), 8.02 (d, 1H), 7.85 (d, 1H), 7.74-7.83 (m, 2H), 7.42-7.53 (m, 2H), 7.38 (t, 1H), 7.30 (d, 1H), 7.23 (t, 1H), 3.93-4.05 (m, 2H), 3.52-3.62 (m, 2H), 2.97-3.10 (m, 2H), 2.84 (t, 2H), 2.56 (t, 2H), 2.23 (s, 3H), 1.88-2.00 (m, 2H), 1.45 (s, 2H), 1.25-1.39 (m, 4H), 1.12-1.22 (m, 4H), 1.00-1.09 (m, 2H), 0.89 (s, 6H). MS (ESI) m/e 760.1 (M+H)⁺.

### 1.2. Synthesis of 6-[4-(1,3-benzothiazol-2-ylcarbamoyl)-3,4-dihydro-2H-1,4-benzoxazin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.02)

### 1.2.1. tert-butyl 3-(1-(((-3-(2-((tert-butoxycarbonyl)(methyl) amino)ethoxy)-5,7-dimethyladamantan-1-yl)methyl)-5-methyl-1H-pyrazol-4-yl)-6-(3,4-dihydro-2H-benzo[b][1,4]oxazin-6-yl)picolinate

To a solution of 6-(4,4,5,5-tetramethyl-1,3,2-dioxaborolan-2-yl)-3,4-dihydro-2H-benzo[b][1,4]oxazine (122 mg) in dioxane (4 mL) and water (1 mL) was added Example 1.1.11 (300 mg), bis(triphenylphosphine)palladium(II) dichloride (32.7 mg), and CsF (212 mg). The mixture was stirred at reflux overnight. The mixture was diluted with ethyl acetate (500 mL) and washed with water, brine and dried over Na₂SO₄. Filtration and evaporation of the solvents gave crude material which was purified via column chromatography (20% ethyl acetate in heptane followed by 5% methanol in dichloromethane) to provide the title compound. MS (ESI) m/e 742.4 (M+H)⁺.

### 1.2.2. 6-[4-(1,3-benzothiazol-2-ylcarbamoyl)-3,4-dihydro-2H-1,4-benzoxazin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

To an ambient suspension of bis(2,5-dioxopyrrolidin-1-yl) carbonate (70.4 mg) in acetonitrile (4 mL) was added benzo[d]thiazol-2-amine (41.3 mg) and the mixture was stirred for one hour. A solution of Example 1.2.1 (170 mg) in acetonitrile (1 mL) and water (10 mL) was added, and the suspension was stirred vigorously overnight. The mixture was diluted with ethyl acetate (500 mL) and washed with water, brine and dried over Na₂SO₄. Filtration and evaporation of the solvents afforded a residue which was loaded on a column and eluted with 20% ethyl acetate in heptane followed by 5% methanol in dichloromethane. The resultant material was treated with 20% TFA in dichloromethane overnight. After evaporation of the solvent, the residue was purified via HPLC (Gilson system, eluting with 10-85% acetonitrile in 0.1% TFA in water) to provide the title compound. ¹H NMR (400 MHz, dimethyl sulfoxide-d₆) δ ppm 8.76 (s, 1H), 8.24-8.46 (m, 2H), 7.97 (d, 1H), 7.70-7.89 (m, 3H), 7.47 (s, 1H), 7.35-7.47 (m, 2H), 7.24 (t, 1H), 7.02 (d, 1H), 4.32-4.42 (m, 3H), 4.14-4.23 (m, 3H), 3.90 (s, 3H), 3.57 (t, 3H), 2.93-3.11 (m, 2H), 2.57 (t, 3H), 2.23 (s, 3H), 1.46 (s, 2H), 1.24-1.39 (m, 4H), 0.98-1.25 (m, 5H), 0.89 (s, 6H). MS (ESI) m/e 760.4 (M+H)⁺.

### 1.3. Synthesis of 6-[4-(1,3-benzothiazol-2-ylcarbamoyl)-1-methyl-1,2,3,4-tetrahydroquinoxalin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.03)

### 1.3.1. tert-butyl 3-(1-((3-(2-((tert-butoxycarbonyl)(methyl)amino) ethoxy)-5,7-dimethyladamantan-1-yl)methyl)-5-methyl-1H-pyrazol-4-yl)-6-(1-methyl-1,2,3,4-tetrahydroquinoxalin-6-yl)picolinate

To a solution of 1-methyl-6-(4,4,5,5-tetramethyl-1,3,2-dioxaborolan-2-yl)-1,2,3,4-tetrahydroquinoxaline (140 mg) in dioxane (4 mL) and water (1 mL) was added Example 1.1.11 (328 mg), bis(triphenylphosphine)palladium(II) dichloride (35.8 mg), and CsF (232 mg). The mixture was stirred at reflux overnight. The mixture was diluted with ethyl acetate (500 mL) and washed with water, brine and dried over Na₂SO₄. Filtration and evaporation of the solvent gave crude material which was purified via column chromatography, eluting with 20% ethyl acetate in heptane followed by 5% methanol in dichloromethane, to provide the title compound. MS (ESI) m/e 755.5 (M+H)⁺.

### 1.3.2. 6-[4-(1,3-benzothiazol-2-ylcarbamoyl)-1-methyl-1,2,3,4-tetrahydroquinoxalin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

To an ambient suspension of bis(2,5-dioxopyrrolidin-1-yl) carbonate (307 mg) in acetonitrile (10 mL) was added benzo[d]thiazol-2-amine (180 mg) and the mixture was stirred for one hour. A solution of Example 1.3.1 (600 mg) in acetonitrile (3 mL) was added, and the suspension was vigorously stirred overnight. The mixture was diluted with ethyl acetate (500 mL) and washed with water and brine and dried over Na₂SO₄. Filtration and evaporation of the solvents afforded a residue which was loaded on a column and eluted with 20% ethyl acetate in heptane (1 L) followed by 5% methanol in dichloromethane. The resultant material was treated with 20% TFA in dichloromethane overnight. After evaporation of solvent, the residue was purified on an HPLC (Gilson system, eluting with 10-85% acetonitrile in 0.1% TFA in water) to give the title compound. ¹H NMR (400 MHz, dimethyl sulfoxide-d₆) δ ppm 8.17-8.44 (m, 3H), 7.90 (d, 1H), 7.68-7.84 (m, 3H), 7.45 (s, 2H), 7.37 (t, 1H), 7.22 (t, 1H), 6.83 (d, 1H), 3.96-4.12 (m, 2H), 3.89 (s, 3H), 3.57 (t, 2H), 3.44 (t, 2H), 2.93-3.09 (m, 4H), 2.56 (t, 3H), 2.21 (s, 3H), 1.45 (s, 2H), 1.25-1.39 (m, 4H), 0.99-1.22 (m, 7H), 0.89 (s, 6H). MS (ESI) m/e 760.4 (M+H)⁺.

### 1.4. Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid (Compound W3.04)

### 1.4.1. 2-((3-((4-iodo-5-methyl-1H-pyrazol-1-yl)methyl)-5,7-dimethyladamantan-1-yl)oxy)ethanamine

A solution of Example 1.1.7 (4.5 g) in 7N ammonium in methanol (15 mL) was stirred at 100° C. for 20 minutes under microwave conditions (Biotage Initiator). The reaction mixture was concentrated under vacuum. The residue was diluted with ethyl acetate (400 mL) and washed with aqueous NaHCO₃, water (60 mL) and brine (60 mL). The organic layer was dried (anhydrous Na₂SO₄), the solution was filtered and concentrated, and the residue was used in the next reaction without further purification. MS (ESI) m/e 444.2 (M+H)⁺.

### 1.4.2. tert-butyl (2-((3-((4-iodo-5-methyl-1H-pyrazol-1-yl)methyl)-5,7-dimethyladamantan-1-yl)oxy)ethyl)carbamate

To a solution of Example 1.4.1 (4.4 g) in tetrahydrofuran (100 mL) was added di-tert-butyl dicarbonate (2.6 g) and N,N-dimethyl-4-aminopyridine (100 mg). The mixture was stirred for 1.5 hours. The reaction mixture was diluted with ethyl acetate (300 mL) and washed with aqueous NaHCO₃, water (60 mL) and brine (60 mL). After drying (anhydrous Na₂SO₄), the solution was filtered and concentrated, and the residue was purified by silica gel column chromatography (20% ethyl acetate in dichloromethane) to give the title compound. MS (ESI) m/e 544.2 (M+H)⁺.

### 1.4.3. 6-fluoro-3-bromopicolinic acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour. The resulting mixture was stirred for another 30 minutes, warmed to 35° C., and stirred overnight. The reaction mixture was cooled to room temperature and adjusted to pH 4 with a NaH₂PO₄ solution. The resulting solution was extracted three times with dichloromethane, and the combined extracts were washed with brine, dried over sodium sulfate, filtered and concentrated to provide the title compound.

### 1.4.4. Tert-butyl 3-bromo-6-fluoropicolinate

Para-toluenesulfonyl chloride (27.6 g) was added to a solution of Example 1.4.3 (14.5 g), pyridine (26.7 mL) and tert-butanol (80 mL) in dichloromethane (100 mL) at 0° C. The reaction was stirred for 15 minutes, warmed to room temperature, and stirred overnight. The solution was concentrated and partitioned between ethyl acetate and Na₂CO₃ solution. The layers were separated, and the aqueous layer was extracted with ethyl acetate. The organic layers were combined, rinsed with Na₂CO₃ solution and brine, dried over sodium sulfate, filtered, and concentrated to provide the title compound.

### 1.4.5. Ethyl 7-(5-bromo-6-(tert-butoxycarbonyl)pyridin-2-yl)-5,6,7,8-tetrahydroimidazo[1,5-a]pyrazine-1-carboxylate

Ethyl 5,6,7,8-tetrahydroimidazo[1,5-a]pyrazine-1-carboxylate hydrochloride (692 mg) and Example 1.4.4 (750 mg) were dissolved in dimethyl sulfoxide (6 mL). N,N-Diisopropylethylamine (1.2 mL) was added, and the solution was heated at 50° C. for 16 hours. The solution was cooled, diluted with water (20 mL), and extracted with ethyl acetate (50 mL). The organic portion was washed with brine and dried on anhydrous sodium sulfate. The solution was concentrated and, upon standing for 16 hours, solid crystals formed. The crystals were washed with diethyl ether to yield the title compound. MS (ESI) m/e 451, 453 (M+H)⁺, 395, 397 (M-tert-butyl)⁺.

### 1.4.6. Ethyl 7-(6-(tert-butoxycarbonyl)-5-(4,4,5,5-tetramethyl-1,3,2-dioxaborolan-2-yl)pyridin-2-yl)-5,6,7,8-tetrahydroimidazo[1,5-a]pyrazine-1-carboxylate

The title compound was prepared by substituting Example 1.4.5 for Example 1.1.9 in Example 1.1.10. MS (ESI) m/e 499 (M+H)⁺, 443 (M−tert-butyl)⁺, 529 (M+MeOH−H)⁻.

### 1.4.7. Ethyl 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5,6,7,8-tetrahydroimidazo[1,5-a]pyrazine-1-carboxylate

Example 1.4.6 (136 mg) and Example 1.4.2 (148 mg) were dissolved in 1,4-dioxane (3 mL) and water (0.85 mL). Tripotassium phosphate (290 mg) was added, and the solution was degassed and flushed with nitrogen three times. Tris(dibenzylideneacetone)dipalladium(0) (13 mg) and 1,3,5,7-tetramethyl-8-tetradecyl-2,4,6-trioxa-8-phosphaadamantane (12 mg) were added. The solution was degassed, flushed with nitrogen once, and heated to 70° C. for 16 hours. The reaction was cooled and diluted with ethyl acetate (10 mL) and water (3 mL). The layers were separated, and the organic layer was washed with brine and dried on anhydrous sodium sulfate. After filtration, the filtrate was concentrated and purified by flash column chromatography on silica gel, eluting with 5% methanol in ethyl acetate. The solvent was removed under reduced pressure to give the title compound. MS (ESI) m/e 760 (M+H)⁺, 758 (M−H)⁻.

### 1.4.8. 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5,6,7,8-tetrahydroimidazo[1,5-a]pyrazine-1-carboxylic acid

Example 1.4.7 (200 mg) was dissolved in tetrahydrofuran (0.7 mL), methanol (0.35 mL), and water (0.35 mL). Lithium hydroxide monohydrate (21 mg) was added, and the solution was stirred at room temperature for 16 hours. HCl (1M, 0.48 mL) was added and the water was removed by azeotroping twice with ethyl acetate (20 mL). The solvent was removed under reduced pressure, and the material was dried under vacuum. The material was dissolved in dichloromethane (5 mL) and ethyl acetate (1 mL) and dried over anhydrous sodium sulfate. After filtration, the solvent was removed under reduced pressure to give the title compound. MS (ESI) m/e 760 (M+H)⁺, 758 (M−H)⁻.

### 1.4.9. Tert-butyl 6-(1-(benzo[d]thiazol-2-ylcarbamoyl)-5,6-dihydroimidazo[1,5-a]pyrazin-7(8H)-yl)-3-(1-((3-(2-((tert-butoxycarbonyl)amino)ethoxy)-5,7-dimethyladamantan-1-yl)methyl)-5-methyl-1H-pyrazol-4-yl)picolinate

Example 1.4.6 (160 mg) and benzo[d]thiazol-2-amine (35 mg) were dissolved in dichloromethane (1.5 mL). 1-Ethyl-3-[3-(dimethylamino)propyl]-carbodiimide hydrochloride (85 mg) and 4-(dimethylamino)pyridine (54 mg) were added, and the solution was stirred at room temperature for 16 hours. The material was purified by flash column chromatography on silica gel, eluting with 2.5-5% methanol in ethyl acetate. The solvent was removed under reduced pressure to give the title compound. MS (ESI) m/e 892 (M+H)⁺, 890 (M−H)⁻.

### 1.4.10. 3-(1-{[3-(2-aminoethoxy)-5,7-dimethyltricyclo[3.3.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid

The title compound was prepared by substituting Example 1.4.9 for Example 1.1.13 in Example 1.1.14. ¹H NMR (400 MHz, dimethyl sulfoxide-d₆) δ ppm 11.50 (bs, 1H), 8.21 (d, 1H), 7.98 (d, 1H), 7.93 (s, 1H), 7.76 (d, 1H), 7.66 (bs, 3H), 7.58 (d, 1H), 7.44 (t, 1H), 7.33 (s, 1H), 7.31 (t, 1H), 7.15 (d, 1H), 6.97 (d, 1H), 5.10 (s, 2H), 4.26 (m, 2H), 4.08 (t, 2H), 3.84 (s, 2H), 2.90 (m, 4H), 2.13 (s, 3H), 1.42 (s, 2H), 1.30 (q, 4H), 1.15 (m, 2H), 1.04 (q, 4H), 0.87 (s, 6H). MS (ESI) m/e 736 (M+H)⁺, 734 (M−H)⁻.

### 1.5. Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-hydroxy-3,4-dihydroisoquinolin-2(1H)-yl]pyridine-2-carboxylic acid (Compound W3.05)

### 1.5.1. tert-butyldiphenyl(vinyl)silane

The title compound was prepared as described in J Org Chem, 70(4), 1467 (2005).

### 1.5.2. 2-(tert-butyldiphenylsilyl)ethanol

Example 1.5.1 (8.2 g) was dissolved in tetrahydrofuran (30 mL), then a 0.5M solution of 9-borabicyclo[3.3.1]nonane in tetrahydrofuran (63 mL) was added and the reaction was stirred at room temperature for 2.5 hours. The reaction was warmed to 37° C., then 3.ON aqueous NaOH (11 mL) was added, followed by the very careful dropwise addition of 30% aqueous H₂O₂(11 mL). Once the peroxide addition was completed, the reaction was stirred for one hour, and water (200 mL) and diethyl ether (200 mL) were added. The organic layer was washed with brine and dried over sodium sulfate. After filtration and concentration, purification by silica gel chromatography, eluting with heptanes/ethyl acetate (3/1), gave the title compound.

### 1.5.3. 5-(2-(tert-butyldiphenylsilyl)ethoxy)isoquinoline

Triphenylphosphine (262 mg) was dissolved in tetrahydrofuran (2 mL). Example 1.5.2 (285 mg), isoquinolin-5-ol (121 mg), and diisopropyl azodicarboxylate (203 mg) were added. The reaction was stirred at room temperature for 30 minutes, then more isoquinolin-5-ol (41 mg) was added and the reaction was stirred overnight. The reaction was then concentrated and purification by flash chromatography, eluting with heptanes/ethyl acetate (83/17), gave the title compound. MS (DCI) m/e 412.2 (M+H)⁺.

### 1.5.4. 8-bromo-5-(2-(tert-butyldiphenylsilyl)ethoxy)isoquinoline

Example 1.5.3 (6.2 g) was dissolved in acetic acid (40 mL), and sodium acetate (2.2 g) was added. A solution of bromine (0.70 mL) in acetic acid (13 mL) was added slowly. The reaction was stirred at room temperature overnight. The reaction was carefully added to 2M aqueous Na₂CO₁ and extracted with ethyl acetate. The organic layer was washed with brine and dried over sodium sulfate. After filtration and concentration, purification by silica gel chromatography, eluting with heptanes/ethyl acetate (9/1), gave the title compound. MS (DCI) m/e 490.1. 492.1 (M+H)⁺.

### 1.5.5. 8-bromo-5-(2-(tert-butyldiphenylsilyl)ethoxy)-1,2,3,4-tetrahydroisoquinoline

Example 1.5.4 (4.46 g) was dissolved in methanol (45 mL). Sodium cyanoborohydride (2.0 g) was added followed by trifluoroborane etherate (4.0 mL, 31.6 mmol). The mixture was heated under reflux for two hours and then cooled to room temperature. Additional sodium cyanoborohydride (2.0 g) and trifluoroborane etherate (4.0 mL) were added, and the mixture was heated under reflux for two more hours. The reaction was cooled, then added to 1/1 water/2M aqueous Na₂CO₃ (150 mL). The mixture was extracted with dichloromethane (twice with 100 mL). The organic layer was dried over sodium sulfate. Filtration and concentration provided the title compound that was used in the next step with no further purification. MS (DCI) m/e 494.1, 496.1 (M+H)⁺.

### 1.5.6. tert-butyl 8-bromo-5-(2-(tert-butyldiphenylsilyl)ethoxy)-3,4-dihydroisoquinoline-2(1H)-carboxylate

Example 1.5.5 (3.9 g) was dissolved in dichloromethane (25 mL), and triethylamine (3.3 mL) and di-tert-butyl dicarbonate (1.9 g) were added. The reaction mixture was stirred at room temperature for three hours. The reaction was then concentrated and purified by flash chromatography, eluting with heptanes/ethyl acetate (96/4), to provide the title compound.

### 1.5.7. 2-tert-butyl 8-methyl 5-(2-(tert-butyldiphenylsilyl)ethoxy)-3,4-dihydroisoquinoline-2,8(1H)-dicarboxylate

Example 1.5.6 (3.6 g) and [1,1′-bis(diphenylphosphino)ferrocene]dichloropalladium(II) dichloromethane (0.025 g) were placed in a 250 mL SS pressure bottle, and methanol (10 mL) and triethylamine (0.469 mL) were added. After degassing the reactor with argon several times, the flask was charged with carbon monoxide and heated to 100° C. for 16 hours at 40 psi. The reaction mixture was cooled, concentrated, and purified by flash silica gel chromatography, eluting heptanes/ethyl acetate (88/12), to provide the title compound.

### 1.5.8. methyl 5-(2-(tert-butyldiphenylsilyl)ethoxy)-1,2,3,4-tetrahydroisoquinoline-8-carboxylate

Example 1.5.7 (1.8 g) was dissolved in 4N HCl in dioxane (25 mL) and stirred at room temperature for 45 minutes. The reaction was then concentrated to provide the title compound as a hydrochloride salt. MS (DCI) m/e 474.2 (M+H)⁺.

### 1.5.9. methyl 2-(5-bromo-6-(tert-butoxycarbonyl)pyridin-2-yl)-5-(2-(tert-butyldiphenylsilyl)ethoxy)-1,2,3,4-tetrahydroisoquinoline-8-carboxylate

To a solution of Example 1.5.8 (1.6 g) and Example 1.4.4 (1.0 g) in dimethyl sulfoxide (6 mL) was added N,N-diisopropylethylamine (1.4 mL). The mixture was stirred at 50° C. for 24 hours. The mixture was then diluted with diethyl ether and washed with water and brine, and dried over Na₂SO₄. Filtration and evaporation of the solvent and silica gel column purification (eluting with 5% ethyl acetate in hexane) gave the title compound.

### 1.5.10. 1-((3-(2-azidoethoxy)-5,7-dimethyladamantan-1-yl)methyl)-4-iodo-5-methyl-1H-pyrazole

Example 1.1.6 (2 g) was dissolved in dichloromethane (20 mL), and triethylamine (0.84 mL) was added. After cooling the reaction solution to 5° C., mesyl chloride (0.46 mL) was added dropwise. The cooling bath was removed and the reaction was stirred at room temperature for two hours. Saturated NaHCO₃ was added, the layers were separated, and the organic layer was washed with brine, and dried over Na₂SO₄. After filtration and concentration, the residue was dissolved in N,N dimethylformamide (15 mL) and sodium azide (0.88 g) was added, and the reaction was heated to 80° C. for two hours. The reaction was then cooled to room temperature and poured into diethyl ether and water. The organic layer was separated and washed with brine and dried over Na₂SO₄. After filtration and concentration, purification by silica gel chromatography, eluting with heptanes/ethyl acetate (4/1), gave the title compound. MS (DCI) m/e 470.0 (M+H)⁺.

### 1.5.11. methyl 2-(6-(tert-butoxycarbonyl)-5-(4,4,5,5-tetramethyl-1,3,2-dioxaborolan-2-yl)pyridin-2-yl)-5-(2-(tert-butyldiphenylsilyl)ethoxy)-1,2,3,4-tetrahydroisoquinoline-8-carboxylate

Example 1.5.9 (1.5 g), 4,4,5,5-tetramethyl-1,3,2-dioxaborolane (0.46 mL), [1,1′-bis(diphenylphosphino)ferrocene]dichloropalladium(II) dichloromethane (86 mg), and triethylamine (0.59 mL) were dissolved in acetonitrile (6.5 mL) under a nitrogen atmosphere, then the reaction was heated under reflux overnight. The reaction was then cooled to room temperature and ethyl acetate and water were added. The organic layer was washed with brine and dried over Na₂SO₄. After filtration and concentration, purification by silica gel chromatography, using a gradient of 10-20% ethyl acetate in heptanes, gave the title compound. MS (ESI) m/e 777.1 (M+H)⁺.

### 1.5.12. methyl 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-5-(2-(tert-butyldiphenylsilyl) ethoxy)-1,2,3,4-tetrahydroisoquinoline-8-carboxylate

Example 1.5.11 (1.22 g) and Example 1.5.10 (0.74 g) were dissolved in tetrahydrofuran (16 mL) under a nitrogen atmosphere, and tripotassium phosphate (4.5 g) and water (5 mL) were added. Tris(dibenzylideneacetone)dipalladium(0) (70 mg) and 1,3,5,7-tetramethyl-8-tetradecyl-2,4,6-trioxa-8-phosphaadamantane (66 mg) were then added, the reaction was heated at reflux overnight, and then allowed to cool to room temperature. Ethyl acetate and water were then added, and the organic layer washed with brine and dried over Na₂SO₄. After filtration and concentration, the crude material was purified by silica gel chromatography, eluting with heptanes/ethyl acetate (7/3), gave the title compound. MS (DCI) m/e 992.3 (M+H)⁺.

### 1.5.13. 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-5-(2-(tert-butyldiphenylsilyl)ethoxy)-1,2,3,4-tetrahydroisoquinoline-8-carboxylic acid

Example 1.5.12 (1.15 g) was dissolved in tetrahydrofuran (4.5 mL), and methanol (2.2 mL), water (2.2 mL), and lithium hydroxide monohydrate (96 mg) were added. The reaction mixture was stirred at room temperature for five days. Water (20 mL) and 2N aqueous HCl (1.1 mL) were added. The mixture was extracted with ethyl acetate, and the organic layer was washed with brine and dried over Na₂SO₄. After filtration and concentration, purification by silica gel chromatography, eluting with dichloromethane/ethyl acetate (70/30) followed by dichloromethane/ethyl acetate/acetic acid (70/30/1), gave the title compound.

### 1.5.14. tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-5-(2-(tert-butyldiphenylsilyl)ethoxy)-3,4-dihydroisoquinolin-2(1H)-yl)picolinate

Example 1.5.13 (80 mg) and benzo[d]thiazol-2-amine (14 mg) were dissolved in dichloromethane (1.2 mL). N,N-Dimethylpyridin-4-amine (17 mg) and N-ethyl-N′-(3-dimethylaminopropyl)carbodiimide hydrochloride (27 mg) were added and the reaction was stirred at room temperature overnight. The reaction was concentrated and the crude residue was purified by silica gel chromatography, eluting with dichloromethane/ethyl acetate (90/10), to provide the title compound. MS (ESI) m/e 1110.3 (M+H)⁺.

### 1.5.15. tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-5-hydroxy-3,4-dihydroisoquinolin-2(1H)-yl)picolinate

Example 1.5.14 (160 mg) was dissolved in a 1.0M solution of tetrabutylammonium fluoride in 95/5 tetrahydrofuran/water (1.15 mL) and the reaction was heated at 60° C. for two days. Powdered 4 Å molecular sieves were added, and the mixture was heated at 60° C. for another day. The reaction was cooled, then concentrated and the crude residue was purified by silica gel chromatography, eluting with 70/30/1 dichloromethane/ethyl acetate/acetic acid, to provide the title compound. MS (ESI) m/e 844.2 (M+H)⁺.

### 1.5.16. tert-butyl 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-5-hydroxy-3,4-dihydroisoquinolin-2(1H)-yl)picolinate

Example 1.5.15 (70 mg) was dissolved in tetrahydrofuran (2 mL), 10% palladium on carbon (20 mg) was added, and the mixture was stirred under a hydrogen balloon overnight. After filtration through diatomaceous earth and evaporation of the solvent, the crude title compound was purified by reverse phase chromatography (C18 column), eluting with 10-90% acetonitrile in 0.1% TFA water, to provide the title compound as a trifluoroacetic acid salt.

### 1.5.17. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-5-hydroxy-3,4-dihydroisoquinolin-2(1H)-yl)picolinic acid

Example 1.5.16 (11 mg) was dissolved in 4N HCl in dioxane (0.5 mL) and stirred at room temperature overnight. The solids were filtered off and washed with dioxane to provide the title compound as a hydrochloride salt. ¹H NMR (500 MHz, dimethyl sulfoxide-d₆) δ ppm 12.60 (v br s, 1H), 10.40 (br s, 1H), 8.00 (d, 1H) 7.76 (d, 1H), 7.75 (br s, 3H), 7.60 (d, 1H), 7.51 (d, 1H), 7.46 (t, 1H), 7.33 (t, 1H), 7.30 (s, 1H), 6.98 (d, 1H), 6.82 (d, 1H), 4.99 (s, 2H), 3.89 (m, 2H), 3.83 (s, 2H), 3.50 (m, 2H), 2.88 (m, 2H), 2.79 (m, 2H), 2.11 (s, 3H), 1.41 (s, 2H), 1.29 (m, 4H), 1.14 (m, 4H), 1.04 (m, 2H), 0.87 (s, 6H). MS (ESI) m/e 762.2 (M+H)⁺.

### 1.6. Synthesis of 6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.06)

### 1.6.1. tert-butyl 3-(1-((3-(2-((tert-butoxycarbonyl)(methyl)amino) ethoxy)-5,7-dimethyladamantan-1-yl)methyl-1H-pyrazol-4-yl)-6-(8-(methoxycarbonyl)naphthalen-2-yl)picolinate

To a solution of methyl 7-(4,4,5,5-tetramethyl-1,3,2-dioxaborolan-2-yl)-1-naphthoate (2.47 g) in dioxane (40 mL) and water (20 mL) was added Example 1.1.11 (4.2 g), bis(triphenylphosphine)palladium(II) dichloride (556 mg), and CsF (3.61 g). The mixture was stirred at reflux overnight. The mixture was diluted with ethyl acetate (400 mL) and washed with water and brine, and dried over Na₂SO₄. After filtration and evaporation of the solvent, the crude material was purified via column chromatography, eluting with 20% ethyl acetate in heptane followed by 5% methanol in dichloromethane, to provide the title compound. MS (ESI) m/e 793.4 (M+H)⁺.

### 1.6.2. 7-(6-(tert-butoxycarbonyl)-5-(1-((3-(2-((tert-butoxycarbonyl) (methyl)amino)ethoxy)-5,7-dimethyladamantan-1-yl)methyl)-5-methyl-1H-pyrazol-4-yl)pyridin-2-yl)-1-naphthoic acid

To a solution of Example 1.6.1 (500 mg) in tetrahydrofuran (4 mL), methanol (2 mL) and water (2 mL) was added lithium hydroxide monohydrate (500 mg). The mixture was stirred for 3 hours. The mixture was then acidified with 1N aqueous HCl and diluted with ethyl acetate (200 mL). The organic layer was washed with water and brine, and dried over Na₂SO₄. Filtration and evaporation of the solvent gave the crude title compound which was used in the next reaction without further purification. MS (ESI) m/e 779.4 (M+H)⁺.

### 1.6.3. 6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

To a solution of Example 1.6.2 (79 mg) in N,N-dimethylformamide (2 mL) was added benzo[d]thiazol-2-amine (23 mg), fluoro-N,N,N′,N′-tetramethylformamidinium hexafluorophosphate (41 mg) and N,N-diisopropylethylamine (150 mg). The mixture was stirred at 60° C. for 3 hours. The reaction mixture was diluted with ethyl acetate (200 mL) and washed with water and brine, and dried over Na₂SO₄. Filtration and evaporation of the solvent gave a crude intermediate which was dissolved in dichloromethane/TFA (1:1, 6 mL) and left to sit overnight. Evaporation of the solvent gave a residue which was dissolved in dimethyl sulfoxide/methanol (1:1, 9 mL) and purified by HPLC (Gilson system, eluting with 10-85% acetonitrile in 0.1% TFA in water) to give the pure title compound. ¹H NMR (501 MHz, dimethyl sulfoxide-d₆) δ ppm 13.11 (s, 1H), 9.02 (s, 1H), 8.38 (dd, 1H), 8.26-8.34 (m, 2H), 8.13-8.27 (m, 3H), 8.07 (d, 1H), 8.02 (d, 1H), 7.93 (d, 1H), 7.82 (d, 1H), 7.67-7.75 (m, 1H), 7.44-7.53 (m, 2H), 7.30-7.41 (m, 1H), 3.90 (s, 3H), 2.94-3.12 (m, 3H), 2.53-2.60 (m, 4H), 2.20-2.31 (m, 3H), 1.45 (s, 2H), 1.25-1.39 (m, 4H), 0.99-1.23 (m, 4H), 0.89 (s, 6H). MS (ESI) m/e 755.4 (M+H)⁺.

### 1.7. Synthesis of 3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[5,4-b]pyridin-2-ylcarbamoyl)naphthalen-2-yl]pyridine-2-carboxylic acid (Compound W3.07)

The title compound was prepared by substituting thiazolo[5,4-b]pyridin-2-amine for benzo[d]thiazol-2-amine in Example 1.6.3. ¹H NMR (400 MHz, dimethyl sulfoxide-d₆) δ ppm 13.25 (s, 1H), 9.02 (s, 1H), 8.54 (dd, 1H), 8.39 (dd, 1H), 8.14-8.35 (m, 6H), 8.04 (d, 1H), 7.93 (d, 1H), 7.66-7.75 (m, 1H), 7.55 (dd, 1H), 7.49 (s, 1H), 3.57 (t, 3H), 2.95-3.10 (m, 2H), 2.51-2.62 (m, 3H), 2.19-2.28 (m, 3H), 1.45 (s, 2H), 1.24-1.38 (m, 4H), 0.98-1.24 (m, 6H), 0.89 (s, 6H). MS (ESI) m/e 756.3 (M+H)⁺.

### 1.8. Synthesis of 3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[4,5-b]pyridin-2-ylcarbamoyl)naphthalen-2-yl]pyridine-2-carboxylic acid (Compound W3.08)

The title compound was prepared by substituting thiazolo[4,5-c]pyridin-2-amine for benzo[d]thiazol-2-amine in Example 1.6.3. ¹H NMR (501 MHz, dimethyl sulfoxide-d₆) δ ppm 13.40 (s, 1H), 9.04 (s, 1H), 8.62 (dd, 1H), 8.56 (dd, 1H), 8.39 (dd, 1H), 8.13-8.34 (m, 5H), 8.06 (d, 1H), 7.94 (d, 1H), 7.68-7.79 (m, 1H), 7.45-7.54 (m, 1H), 7.39 (dd, 1H), 3.90 (s, 3H), 3.54-3.60 (m, 3H), 2.94-3.08 (m, 2H), 2.51-2.60 (m, 4H), 2.18-2.31 (m, 3H), 1.46 (s, 2H), 1.24-1.40 (m, 4H), 1.01-1.21 (m, 6H), 0.83-0.89 (m, 5H). MS (ESI) m/e 756.3 (M+H)⁺.

### 1.9. Synthesis of 6-[8-(1,3-benzothiazol-2-ylcarbamoyl)-5-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.09)

### 1.9.1. tert-butyl 8-bromo-5-hydroxy-3,4-dihydroisoquinoline-2(1H)-carboxylate

To a solution of tert-butyl 5-hydroxy-3,4-dihydroisoquinoline-2(1H)-carboxylate (9 g) in N,N-dimethylformamide (150 mL) was added N-bromosuccinimide (6.43 g). The mixture was stirred overnight and quenched with water (200 mL). The mixture was diluted with ethyl acetate (500 mL) and washed with water and brine, and dried over sodium sulfate. Filtration and evaporation of the solvent gave crude title compound which was used in the next reaction without further purification. MS(ESI) m/e 329.2 (M+H)⁺.

### 1.9.2. tert-butyl 5-(benzyloxy)-8-bromo-3,4-dihydroisoquinoline-2(1H)-carboxylate

To a solution of Example 1.9.1 (11.8 g) in acetone (200 mL) was added benzyl bromide (7.42 g) and K₂CO₃(5 g). The mixture was stirred at reflux overnight. The mixture was concentrated and the residue was partitioned between ethyl acetate (600 mL) and water (200 mL). The organic layer was washed with water and brine, and dried over sodium sulfate. Filtration and evaporation of the solvent gave crude title compound which was purified on a silica gel column and eluted with 10% ethyl acetate in heptane to provide the title compound. MS (ESI) m/e 418.1 (M+H)⁺.

### 1.9.3. 2-tert-butyl 8-methyl 5-(benzyloxy)-3,4-dihydroisoquinoline-2,8(1H)-dicarboxylate

Methanol (100 mL) and triethylamine (9.15 mL) were added to Example 1.9.2 (10.8 g) and [1,1′-bis(diphenylphosphino)ferrocene]dichloropalladium(II) (0.48 g) in a 500 mL stainless steel pressure reactor. The vessel was sparged with argon several times. The reactor was pressurized with carbon monoxide and stirred for 2 hours at 100° C. under 60 psi of carbon monoxide. After cooling, the crude reaction mixture was concentrated under vacuum. The residue was partitioned between ethyl acetate (500 mL) and water (200 mL). The organic layer was further washed with water and brine, and dried over sodium sulfate. After filtration and evaporation of the solvent, the residue was purified on a 330 g silica gel column, eluting with 10-20% ethyl acetate in heptane, to provide the title compound. MS (ESI) m/e 398.1 (M+H)⁺.

### 1.9.4. methyl 5-(benzyloxy)-1,2,3,4-tetrahydroisoquinoline-8-carboxylate hydrochloride

To a solution of Example 1.9.3 (3.78 g) in tetrahydrofuran (20 mL) was added 4N HCl in dioxane (20 mL). The mixture was stirred overnight and the mixture was concentrated under vacuum and the crude title compound was used in the next reaction without further purification. MS(ESI) m/e 298.1 (M+H)⁺.

### 1.9.5. methyl 5-(benzyloxy)-2-(5-bromo-6-(tert-butoxycarbonyl)pyridin-2-yl)-1,2,3,4-tetrahydroisoquinoline-8-carboxylate

To a solution of Example 1.9.4 (3.03 g) in dimethyl sulfoxide (50 mL) was added Example 1.4.4 (2.52 g) and triethylamine (3.8 mL). The mixture was stirred at 60° C. overnight under nitrogen. The reaction mixture was diluted with ethyl acetate (500 mL) and washed with water and brine, and dried over sodium sulfate. After filtration and evaporation of the solvent, the crude material was purified on a silica gel column, eluting with 20% ethyl acetate in heptane, to give the title compound. MS (ESI) m/e 553.1 (M+H)⁺.

### 1.9.6. methyl 5-(benzyloxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.9.5 (2.58 g) in tetrahydrofuran (40 mL) and water (20 mL) was added Example 1.1.10 (2.66 g). 1,3,5,7-tetramethyl-6-phenyl-2,4,8-trioxa--6-phosphaadamante (341 mg), tris(dibenzylideneacetone)dipalladium(0) (214 mg), and K₃PO₄ (4.95 g). The mixture was stirred at reflux for 4 hours. The mixture was diluted with ethyl acetate (500 mL) and washed with water and brine, and dried over sodium sulfate. After filtration and evaporation of the solvent, the crude material was purified on a silica gel column, eluting with 20% ethyl acetate in dichloromethane, to give the title compound. MS (ESI) m/e 904.5 (M+H)⁺.

### 1.9.7. methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-hydroxy-1,2,3,4-tetrahydroisoquinoline-8-carboxylate

Example 1.9.6 (3.0 g) in tetrahydrofuran (60 mL) was added to Pd(OH)₂ (0.6 g, Degussa #E101NE/W. 20% on carbon, 49% water content) in a 250 mL SS pressure bottle. The mixture was agitated for 16 hours under 30 psi of hydrogen gas at 50° C. The mixture was then filtered through a nylon membrane, and the solvent concentrated under vacuum to provide the title compound. MS (ESI) m/e 815.1(M+H)⁺.

### 1.9.8. methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylate

Example 1.9.7 (170 mg) was dissolved in dichloromethane (0.8 mL) and methanol (0.2 mL). To the mixture was added a 2.0M solution of (trimethylsilyl)diazomethane in diethyl ether (0.17 mL) and the reaction was stirred at room temperature overnight. Additional 2.0M (trimethylsilyl)diazomethane in diethyl ether (0.10 mL) was added, and the reaction was allowed to stir for 24 hours. The reaction mixture was then concentrated and the title compound was used without further purification. MS (ESI) m/e 828.2 (M+H)⁺.

### 1.9.9. 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylic acid

The title compound was prepared by substituting Example 1.9.8 for Example 1.5.12 in Example 1.5.13. MS (ESI) m/e 814.1 (M+H)⁺.

### 1.9.10. tert-butyl 6-(8-(benzo[d]thiazol-2-ylcarbamoyl)-5-methoxy-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.9.9 for Example 1.5.13 in Example 1.5.14. MS (ESI) m/e 946.1 (M+H)⁺.

### 1.9.11. 6-(8-(benzo[d]thiazol-2-ylcarbamoyl)-5-methoxy-3,4-dihydroisoquinolin-2(1H)-yl)-3-(1-((3,5-dimethyl-7-(2-(methylamino)ethoxy)adamantan-1-yl)methyl)-5-methyl-1H-pyrazol-4-yl)picolinic acid

The title compound was prepared by substituting Example 1.9.10 for Example 1.5.16 in Example 1.5.17. ¹H NMR (500 MHz, dimethyl sulfoxide-d₆) δ ppm 8.74 (br s, 2H), 8.02 (d, 1H) 7.77 (m, 2H), 7.54 (d, 1H), 7.47 (t, 1H), 7.34 (m, 2H), 7.01 (d, 2H), 5.01 (s, 2H), 3.90 (m, 2H), 3.89 (s, 3H), 3.85 (s, 2H), 3.58 (m, 2H), 3.57 (s, 3H), 2.98 (m, 2H), 2.82 (m, 2H), 2.12 (s, 3H), 1.41 (s, 2H), 1.30 (m, 4H), 1.14 (m, 4H), 1.04 (m, 2H), 0.87 (s, 6H). MS (ESI) m/e 790.2 (M+H)⁺.

### 1.10. Synthesis of 6-[5-(1,3-benzothiazol-2-ylcarbamoyl)quinolin-3-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.10)

### 1.10.1. 3-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)quinoline-5-carboxylic acid

A mixture of 3-bromoquinoline-5-carboxylic acid (300 mg), 4,4,4′,4′,5,5,5′0.5′-octamethyl-2,2′-bi(1,3,2-dioxaborolane) (363 mg), and potassium acetate (350 mg) in dioxane (5 mL) was purged with nitrogen gas for 5 minutes, and PdCl₂(dppf)-CH₂C₂ adduct (58.3 mg) was added. The mixture was heated at 100° C. overnight and cooled. To this mixture was added Example 1.1.11 (510 mg), dichlorobis(triphenylphosphine)-palladium(II) (83 mg), CsF (362 mg), and water (3 mL). The resulting mixture was heated at 100° C. overnight and filtered through diatomaceous earth. The filtrate was concentrated, and the residue was dissolved in dimethyl sulfoxide, loaded onto a C18 column (300 g), and eluted with a gradient of 50-100% acetonitrile in a 0.1% TFA/water solution to provide the title compound. MS (ESI) m/e 780.5 (M+H)⁺.

### 1.10.2. tert-butyl 6-(5-(benzo[d]thiazol-2-ylcarbamoyl)quinolin-3-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a mixture of Example 1.10.1 (120 mg), benzo[d]thiazol-2-amine (46.2 mg), and O-(7-azabenzotriazol-1-yl)-N,N,N′,N′-tetramethyluronium hexafluorophosphate (HATU, 117 mg) in N,N-dimethylformamide (0.5 mL) was added N,N-diisopropylethylamine (134 dl). The mixture was stirred overnight and loaded onto a C18 column (300 g), eluting with a gradient of 50-100% acetonitrile in 0.1% TFA/water solution to provide the title compound. MS (ESI) m/e 913.4 (M+H)⁺.

### 1.10.3. 6-[5-(1,3-benzothiazol-2-ylcarbamoyl)quinolin-3-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

Example 1.10.2 (50 mg) in dichloromethane (3 mL) was treated with trifluoroacetic acid (2 mL) overnight and concentrated. The residue was dissolved in a mixture of dimethyl sulfoxide (5 mL), loaded onto a C18 column (300 g), and eluted with a gradient of 10-70% acetonitrile in 0.1% TFA water solution to provide the title compound. ¹H NMR (400 MHz, dimethyl sulfoxide-d₆) δ ppm 13.22 (s, 1H), 9.73 (d, 1H), 9.41 (s, 1H), 8.34 (dd, 2H), 8.27 (s, 3H), 8.18 (d, 1H), 8.08 (d, 1H), 8.02-7.93 (m, 2H), 7.82 (d, 1H), 7.55-7.46 (m, 2H), 7.38 (t, 1H), 3.91 (s, 2H), 3.03 (p, 2H), 2.59-2.53 (m, 4H), 2.25 (s, 3H), 1.46 (s, 2H), 1.38-1.25 (m, 4H), 1.18 (s, 4H), 1.11-1.01 (m, 2H), 0.89 (s, 6H). MS (ESI) m/e 756.2 (M+H)⁺.

### 1.11. Synthesis of 6-[4-(1,3-benzothiazol-2-ylcarbamoyl)quinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.11)

### 1.11.1. ethyl 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)quinoline-4-carboxylate

The title compound was prepared as described in Example 1.10.1, replacing 3-bromoquinoline-5-carboxylic acid with ethyl 6-bromoquinoline-4-carboxylate. MS (ESI) m/e 808.4 (M+H)⁺.

### 1.11.2. 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)quinoline-4-carboxylic acid

To a solution of Example 1.11.1 (100 mg) in dimethyl sulfoxide (2 mL) was added methanol (2 mL) and 1M lithium hydroxide (248 μl). The mixture was stirred for 30 minutes, acidified to pH 4 with 10% HCl, diluted with ethyl acetate and washed with water and brine to provide the title compound. MS (ESI) m/e 780.4 (M+H)⁺.

### 1.11.3. tert-butyl 6-(4-(benzo[d]thiazol-2-ylcarbamoyl)quinolin-6-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared as described in Example 1.10.2, replacing Example 1.10.1 with Example 1.11.2. MS (ESI) m/e 912.3 (M+H)⁺.

### 1.11.4. 6-[4-(1,3-benzothiazol-2-ylcarbamoyl)quinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared as described in Example 1.10.3, replacing Example 1.10.2 with Example 1.11.3. ¹H NMR (400 MHz, dimethyl sulfoxide-d₆) δ ppm 13.34 (s, 2H), 9.14 (d, 1H), 8.94 (s, 1H), 8.63 (dd, 1H), 8.27 (dd, 4H), 8.09 (d, 1H), 8.00-7.90 (m, 2H), 7.83 (d, 1H), 7.50 (d, 2H), 7.40 (t, 1H), 3.90 (s, 2H), 3.03 (p, 2H), 2.56 (t, 4H), 2.23 (s, 3H), 1.45 (s, 2H), 1.32 (d, 3H), 1.18 (s, 4H), 1.11-0.98 (m, 2H), 0.89 (s, 6H). MS (ESI) m/e 756.2 (M+H)⁺.

### 1.12. Synthesis of 6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid (Compound W3.12)

### 1.12.1. methyl 5-(benzyloxy)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

The title compound was prepared by substituting Example 1.9.5 for Example 1.5.9 in Example 1.5.11. MS (DCI) m/e 601.0 (M+H)⁻.

### 1.12.2. 2-((3-((4-iodo-5-methyl-1H-pyrazol-1-yl)methyl)-5,7-dimethyladamantan-1-yl)oxy)acetaldehyde

Dimethylsulfoxide (4.8 mL) was dissolved in dichloromethane (150 mL). The mixture was cooled to −75° C., and oxalyl chloride (2.6 mL) was added dropwise. The reaction mixture was stirred at −75° C. for 45 minutes, and a solution of Example 1.1.6 (7.1 g) in dichloromethane (45 mL) was added dropwise. The reaction mixture was stirred at −75° C. for 30 minutes, and triethylamine (5.0 mL) was added. The reaction was warmed to room temperature, poured into water, and extracted with diethyl ether. The organic layer was washed with brine and dried over Na₂SO₄. After filtration and concentration, purification by silica gel chromatography, eluting with dichloromethane/ethyl acetate 85/15, gave the title compound. MS (DCI) m/e 443.0 (M+H)⁺.

### 1.123. 2-((3-((4-iodo-5-methyl-1H-pyrazol-1-yl)methyl)-5,7-dimethyladamantan-1-yl)oxy)-N-(2-methoxyethyl)ethanamine

Example 1.12.2 (4.0 g) and 2-methoxyethanamine (0.90 mL) were dissolved in dichloromethane (40 mL) and the mixture was stirred at room temperature for two hours. A suspension of sodium borohydride (500 mg) in methanol (7 mL) was added and the resulting mixture was stirred for 45 minutes. The reaction was then added to saturated aqueous NaHCO₃ and resultant mixture extracted with ethyl acetate. The organic layer was washed with brine and dried over Na₂SO₄. The title compound was obtained after filtration and concentration and was used without purification. MS (DCI) m/e 502.1 (M+H)⁺.

### 1.12.4. tert-butyl (2-((3-((4-iodo-5-methyl-1H-pyrazol-1-yl)methyl)-5,7-dimethyladamantan-1-yl)oxy)ethyl)(2-methoxyethyl)carbamate

Example 1.12.3 (4.4 g) was dissolved in tetrahydrofuran (60 mL), and di-tert-butyl dicarbonate (3.0 g) and N,N-dimethylpyridin-4-amine (0.15 g) were added. The reaction was stirred at room temperature overnight. The reaction was then concentrated and purified by flash chromatography, eluting with dichloromethane/ethyl acetate (3/1), to provide the title compound.

### 1.12.5. methyl 5-(benzyloxy)-2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared by substituting Example 1.12.1 for Example 1.5.11 and Example 1.12.4 for Example 1.5.10 in Example 1.5.12. MS (ESI) m/e 948.2 (M+H)⁺.

### 1.12.6. methyl 2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-5-hydroxy-1,2,3,4-tetrahydroisoquinoline-8-carboxylate

Example 1.12.5 (5.2 g) was dissolved in tetrahydrofuran (100 mL). 20% Palladium hydroxide on activated charcoal (1.0 g) was then added, and the reaction mixture agitated on a Parr rector under a hydrogen atmosphere at 30 psi and 50° C. for 3 hours. After filtration and concentration, purification by silica gel chromatography, eluting with heptanes/ethyl acetate (2/3), gave the title compound. MS (ESI) m/e 858.1 (M+H)⁺.

### 1.12.7. methyl 2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylate

The title compound was prepared by substituting Example 1.12.6 for Example 1.9.7 in Example 1.9.8. MS (ESI) m/e 872.2 (M+H)⁺.

### 1.12.8. 2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylic acid

The title compound was prepared by substituting Example 1.12.7 for Example 1.5.12 in Example 1.5.13. MS (ESI) m/e 858.1 (M+H)⁺.

### 1.12.9. tert-butyl 6-(8-(benzo[d]thiazol-2-ylcarbamoyl)-5-methoxy-3,4-dihydroisoquinolin-2(1H)-yl)-3-(1-((3-(2-((tert-butoxycarbonyl)(2-methoxyethyl)amino)ethoxy)-5,7-dimethyladamantan-1-yl)methyl)-5-methyl-1H-pyrazol-4-yl)picolinate

The title compound was prepared by substituting Example 1.12.8 for Example 1.5.13 in Example 1.5.14. MS (ESI) m/e 990.1 (M+H)⁺.

### 1.12.10.6-(8-(benzo[d]thiazol-2-ylcarbamoyl)-5-methoxy-3,4-dihydroisoquinolin-2(1H)-yl)-3-(1-(((1r,3s,5R,7S)-3-(2-((2-methoxyethyl)amino)ethoxy)-5,7-dimethyladamantan-1-yl)methyl)-5-methyl-H-pyrazol-4-yl)picolinic acid

Example 1.12.9 (2.6 g) was dissolved in dioxane (20 mL), then 4N HCl in dioxane (100 mL) was added, and the reaction was stirred at room temperature overnight. The precipitants were allowed to settle and the supernatant was drawn off. The remaining solids were purified by reverse phase chromatography (C18 column), eluting with 10-90% acetonitrile in 0.1% TFA/water, to provide the title compound as a trifluoroacetic acid salt. ¹H NMR (500 MHz, dimethyl sulfoxide-d₆) δ ppm 8.41 (v br s, 2H), 8.01 (d, 1H) 7.77 (m, 2H), 7.50 (d, 1H), 7.47 (m, 1H), 7.34 (t, 1H), 7.29 (s, 1H), 7.01 (dd, 2H), 5.00 (s, 2H), 3.90 (m, 2H), 3.89 (s, 3H), 3.83 (s, 2H), 3.56 (m, 4H), 3.29 (s, 3H), 3.12 (m, 2H), 3.05 (m, 2H), 2.81 (m, 2H), 2.11 (s, 3H), 1.41 (s, 2H), 1.30 (m, 4H), 1.14 (m, 4H), 1.04 (m, 2H), 0.87 (s, 6H). MS (ESI) m/e 834.3 (M+H)⁺.

### 1.13. Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid (Compound W3.13)

### 1.13.1. 4-Bromo-3-cyanomethyl-benzoic acid methyl ester

Trimethylsilanecarbonitrile (3.59 mL) was added to tetrahydrofuran (6 mL). 1M Tetrabutylammonium fluoride (26.8 mL) was added dropwise over 30 minutes. The solution was then stirred at room temperature for 30 minutes. Methyl 4-bromo-3-(bromomethyl)benzoate (7.50 g) was dissolved in acetonitrile (30 mL) and the resultant solution added to the first solution dropwise over 30 minutes. The solution was then heated to 80° C. for 30 minutes and then allowed to cool to room temperature. The solution was concentrated under reduced pressure and purified by flash column chromatography on silica gel, eluting with 20-30% ethyl acetate in heptanes. The solvent was evaporated under reduced pressure to provide the title compound.

### 1.13.2. 3-(2-Aminoethyl)-4-bromobenzoic acid methyl ester

Example 1.13.1 (5.69 g) was dissolved in tetrahydrofuran (135 mL), and 1 M borane (in tetrahydrofuran, 24.6 mL) was added. The solution was stirred at room temperature for 16 hours and then slowly quenched with methanol and 1M HCL. 4M HCl (150 mL) was added, and the solution was stirred at room temperature for 16 hours. The mixture was concentrated was reduced under reduced pressure, and the pH adjusted to between 11 and 12 using solid potassium carbonate. The solution was then extracted with dichloromethane (3×100 mL). The organic extracts were combined and dried over anhydrous sodium sulfate. The solution was filtered and concentrated under reduced pressure, and the material was purified by flash column chromatography on silica gel, eluting with 10-20% methanol in dichloromethane. The solvent was evaporated under reduced pressure to provide the title compound. MS (ESI) m/e 258, 260 (M+H)⁺.

### 1.13.3. 4-Bromo-3-[2-(2,2,2-trifluoroacetylamino)-ethyl]-benzoic acid methyl ester

Example 1.13.2 (3.21 g) was dissolved in dichloromethane (60 mL). The solution was cooled to 0° C., and triethylamine (2.1 mL) was added. Trifluoroacetic anhydride (2.6 mL) was then added dropwise. The solution was stirred at 0° C. for ten minutes and then allowed to warm to room temperature while stirring for one hour. Water (50 mL) was added and the solution was diluted with ethyl acetate (100 mL). 1M HCl was added (50 mL) and the organic layer was separated, washed with 1M HCl, and then washed with brine. The organic layer was then dried on anhydrous sodium sulfate. After filtration, the solvent was evaporated under reduced pressure to provide the title compound. MS (ESI) m/e 371, 373 (M+H)⁺.

### 1.13.4. 5-Bromo-2-(2,2,2-trifluoroacetyl)-1,2,3,4-tetrahydroisoquinoline-8-carboxylic acid methyl ester

Example 1.13.3 (4.40 g) and paraformaldehyde (1.865 g) were placed in a flask and concentrated sulfuric acid (32 mL) was added. The solution was stirred at room temperature for one hour. Cold water (120 mL) was added. The solution was extracted with ethyl acetate (3×100 mL). The extracts were combined, washed with saturated aqueous sodium bicarbonate (100 mL), washed with water (100 mL), and dried over anhydrous sodium sulfate. The solution was concentrated under reduced pressure, and the material was purified by flash column chromatography on silica gel, eluting with 20-30% ethyl acetate in heptanes. The solvent was evaporated under reduced pressure to provide the title compound. MS (ESI) m/e 366, 368 (M+H)⁺.

### 1.13.5. 5-Cyano-2-(2,2,2-trifluoroacetyl)-1,2,3,4-tetrahydroisoquinoline-8-carboxylic acid methyl ester

Example 1.13.4 (500 mg) and dicyanozinc (88 mg) were added to N,N-dimethylformamide (4 mL). The solution was degassed and flushed with nitrogen three times. Tetrakis(triphenylphosphine)palladium(0) (79 mg) was added, and the solution was degassed and flushed with nitrogen once. The solution was then stirred at 80° C. for 16 hours. The solution was cooled, diluted with 50% ethyl acetate in heptanes (20 mL), and washed with 1 M hydrochloric acid (15 mL) twice. The organic layer was washed with brine and dried over anhydrous sodium sulfate. The solution was filtered and concentrated under reduced pressure, and the material was purified by flash column chromatography on silica gel, eluting with 20-30% ethyl acetate in heptanes. The solvent was evaporated under reduced pressure to provide the title compound.

### 1.13.6. 5-Cyano-1,2,3,4-tetrahydroisoquinoline-8-carboxylic acid methyl ester

Example 1.13.5 (2.00 g) was dissolved in methanol (18 mL) and tetrahydrofuran (18 mL). Water (9 mL) was added followed by potassium carbonate (1.064 g). The reaction was stirred at room temperature for 135 minutes and then diluted with ethyl acetate (100 mL). The solution was washed with saturated aqueous sodium bicarbonate and dried on anhydrous sodium sulfate. The solvent was filtered and evaporated under reduced pressure to provide the title compound. MS (ESI) m/e 217 (M+H)⁺.

### 1.13.7. 2-(5-Bromo-6-tert-butoxycarbonylpyridin-2-yl)-5-cyano-1,2,3,4-tetrahydroisoquinoline-8-carboxylic acid methyl ester

Example 1.13.6 (1.424 g) and Example 1.4.4 (1.827 g) were dissolved in dimethyl sulfoxide (13 mL). N,N-Diisopropylethylamine (1.73 mL) was added, and the solution was heated to 50° C. for 16 hours. Additional Example 1.4.4 (0.600 g) was added, and the solution was heated at 50° C. for another 16 hours. The solution was allowed to cool to room temperature, diluted with ethyl acetate (50 mL), washed with water (25 mL) twice, washed with brine, and then dried on anhydrous sodium sulfate. The solution was filtered and concentrated under reduced pressure, and the material was purified by flash column chromatography on silica gel, eluting with 20-50% ethyl acetate in heptanes. The solvent was evaporated under reduced pressure to provide the title compound. MS (ESI) m/e 472. 474 (M+H)⁺.

### 1.13.8. 2-[6-tert-Butoxycarbonyl-5-(4,4,5,5-tetramethyl-[1,3,2]dioxaborolan-2-yl)-pyridin-2-yl]-5-cyano-1,2,3,4-tetrahydroisoquinoline-8-carboxylic acid methyl ester

Example 1.13.7 (2.267 g) and triethylamine (1.34 mL) were added to acetonitrile (15 mL). The solution was degassed and flushed with nitrogen three times. 4,4,5,5-Tetramethyl-1,3,2-dioxaborolane (1.05 mL) was added followed by dichloro[1,1′-bis(diphenylphosphino)ferrocene]palladium(II) (196 mg). The solution was degassed and flushed with nitrogen once and heated to reflux for 16 hours. The solution was cooled, diluted with ethyl acetate (50 mL), washed with water (10 mL), washed with brine, and dried on anhydrous sodium sulfate. The solution was concentrated under reduced pressure, and the material was purified by flash column chromatography on silica gel, eluting with 20-30% ethyl acetate in heptanes. The solvent was evaporated under reduced pressure to provide the title compound. MS (ESI) m/e 520 (M+H)⁺.

### 1.13.9. 2-(6-tert-Butoxycarbonyl-5-{1-[5-(2-tert-butoxycarbonylamino-ethoxy)-3,7-dimethyl-adamantan-1-ylmethyl]-5-methyl-1H-pyrazol-4-yl}-pyridin-2-yl)-5-cyano-1,2,3,4-tetrahydro-isoquinoline-8-carboxylic acid methyl ester

Example 1.13.8 (140 mg) and Example 1.4.2 (146 mg) were dissolved in tetrahydrofuran (3 mL). Potassium phosphate (286 mg) and water (0.85 mL) were added. The solution was degassed and flushed with nitrogen three times. (1S,3R,5R,7S)-1,3,5,7-Tetramethyl-8-tetradecyl-2,4,6-trioxa-8-phosphaadamantane (11 mg) and tris(dibenzylideneacetone)dipalladium(0) (12 mg) were added, and the solution was degassed and flushed with nitrogen once. The solution was heated to 62° C. for 16 hours. The solution was cooled, then diluted with water (5 mL) and ethyl acetate (25 mL). The organic layer was separated and washed with brine and dried on anhydrous sodium sulfate. The solution was filtered and concentrated under reduced pressure, and the material was purified by flash column chromatography on silica gel, eluting with 30-50% ethyl acetate in heptanes. The solvent was evaporated under reduced pressure to provide the title compound. MS (ESI) m/e 809 (M+H)⁺.

### 1.13.10.2-(6-tert-Butoxycarbonyl-5-{1-[5-(2-tert-butoxycarbonylamino-ethoxy)-3,7-dimethyl-adamantan-1-ylmethyl]-5-methyl-1H-pyrazol-4-yl}-pyridin-2-yl)-5-cyano-1,2,3,4-tetrahydro-isoquinoline-8-carboxylic acid

Example 1.13.9 (114 mg) was dissolved in tetrahydrofuran (0.7 mL) and methanol (0.35 mL). Water (0.35 mL) was added followed by lithium hydroxide monohydrate (11 mg). The solution was stirred at room temperature for 16 hours, and 1 M hydrochloric acid (0.27 mL) was added. Water (1 mL) was added and the solution was extracted with ethyl acetate (5 mL) three times. The extracts were combined and dried on anhydrous sodium sulfate and filtered. The solvent was evaporated under reduced pressure to provide the title compound. MS (ESI) m/e 795 (M+H)⁺.

### 1.13.11.6-[8-(Benzothiazol-2-ylcarbamoyl)-5-cyano-3,4-dihydro-1H-isoquinolin-2-yl]-3--{1-[5-(2-tert-butoxycarbonylamino-ethoxy)-3,7-dimethyl-adamantan-1-ylmethyl]-5-methyl-1H-pyrazol-4-yl}-pyridine-2-carboxylic acid tert-butyl ester

Example 1.13.10 (89 mg) and benzo[d]thiazol-2-amine (18 mg) were dissolved in dichloromethane (1.2 mL). N-(3-Dimethylaminopropyl)-N′-ethylcarbodiimide hydrochloride (39 mg) and N,N-dimethylpyridin-4-amine (25 mg) were added, and the solution was stirred at room temperature for 16 hours. The material was purified by flash column chromatography on silica gel, eluting with 50% ethyl acetate in heptanes. The solvent was evaporated under reduced pressure to provide the title compound. MS (ESI) m/e 927 (M+H)⁺.

### 1.13.12.3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid

Example 1.13.11 (44 mg) was dissolved in dichloromethane (1 mL). Trifluoroacetic acid (0.144 mL) was added and the solution stirred at room temperature for 16 hours. The solvents were then evaporated under reduced pressure, the residue was dissolved in dichloromethane (1 mL), and the solvent removed under reduced pressure. Diethyl ether was added (2 mL) and was removed under reduced pressure. Diethyl ether (2 mL) was added again and removed under reduced pressure to provide the title compound as the trifluoroacetic acid salt. ¹H NMR (400 MHz, dimethyl sulfoxide-d) δ ppm 8.52 (bs, 1H), 8.05 (d, 1H), 7.92 (d, 1H), 7.82-7.75 (m, 2H), 7.63 (m, 2H), 7.50 (dd, 2H), 7.42-7.28 (m, 3H), 7.16 (t, 1H), 7.04 (d, 1H), 4.98 (s, 2H), 3.96 (t, 2H), 3.83 (s, 2H), 3.49 (t, 2H), 3.15 (t, 2H), 2.90 (q, 2H), 2.10 (s, 3H), 1.41 (s, 2H), 1.35-1.22 (m, 4H), 1.18-0.99 (m, 6H), 0.87 (bs, 6H). MS (ESI) m/e 771 (M+H)⁺.

### 1.14. Synthesis of 6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid (Compound W3.14)

### 1.14.1. 2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethanol

To a solution of Example 1.1.6 (4.45 g) and PdCl₂(dppf)-CH₂Cl₂ adduct (409 mg) in acetonitrile (60 mL) was added triethylamine (5 mL) and pinacolborane (6.4 mL). The mixture was refluxed overnight. The mixture was used directly in the next step without work up. MS (ESI) m/e 444.80 (M+H)⁺.

### 1.14.2. tert-butyl 6-chloro-3-(1-((3-(2-hydroxyethoxy)-5,7-dimethyladamantan-1-yl)methyl)-5-methyl-1H-pyrazol-4-yl)picolinate

To a solution of tert-butyl 3-bromo-6-chloropicolinate (3.06 g) in tetrahydrofuran (50 mL) and water (20 mL) was added Example 1.14.1 (4.45 g), 1,3,5,7-tetramethyl-8-tetradecyl-2,4,6-trioxa-8-phosphaadamantane (0.732 g). Pd2(dba)3 (0.479 g), and K₃PO₄ (11 g). The mixture was stirred at reflux overnight and concentrated. The residue was dissolved in ethyl acetate (500 mL) and washed with water and brine. The organic layer was dried over Na₂SO₄, filtered, and concentrated. The residue was purified by flash chromatography, eluting with a gradient of 20-40% ethyl acetate in dichloromethane, to provide the title compound. MS (ESI) m/e 530.23 (M+H)⁺.

### 1.14.3. tert-butyl 6-chloro-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) stirring solution of Example 1.14.2 (3.88 g) in dichloromethane (30 mL) and triethylamine (6 mL) was added methanesulfonyl chloride (2.52 g). The mixture was stirred at room temperature for 4 hours, diluted with ethyl acetate (400 mL), and washed with water and brine. The organic layer was dried over Na₂SO₄. Filtration and evaporation of the solvents afforded the title compound. MS (ESI) m/e 608.20 (M+H)⁺.

### 1.14.4. tert-butyl 3-(1-((3-(2-aminoethoxy)-5,7-dimethyladamantan-1-yl)methyl)-5-methyl-1H-pyrazol-4-yl)-6-chloropicolinate

A solution of Example 1.14.3 (2.2 g) in 7N ammonium in CH₃OH (20 mL) was heated at 100° C. under microwave conditions (Biotage Initiator) for 45 minutes and concentrated to dryness. The residue was dissolved in ethyl acetate and washed with water and brine. The organic layer was dried over Na₂SO₄, filtered, and concentrated to provide the title compound. MS (ESI) m/e 529.33 (M+H)⁺.

### 1.14.5. tert-butyl 6-chloro-3-(1-((3,5-dimethyl-7-(2-(2-(trimethylsilyl)ethylsulfonamido)ethoxy)adamantan-1-yl)methyl)-5-methyl-1H-pyrazol-4-yl)picolinate

To a cooled (0° C.) solution of Example 1.14.4 (3.0 g) in dichloromethane (30 mL) was added triethylamine (3 mL), followed by 2-(trimethylsilyl)ethanesulfonyl chloride (2.3 g). The mixture was stirred at room temperature for 3 hours and concentrated to dryness. The residue was dissolved in ethyl acetate (400 mL) and washed with aqueous NaHCO₃, water, and brine. The residue was dried over Na₂SO₄, filtered, concentrated, and purified by flash chromatography, eluting with 20% ethyl acetate in heptane, to provide the title compound. MS (ESI) m/e 693.04 (M+H)⁺.

### 1.14.6. tert-butyl 6-chloro-3-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)picolinate

To a solution of Example 1.14.5 (415 mg) in toluene (15 mL) was added 2-methoxyethanol (91 mg), followed by cyanomethylenetributylphosphorane (289 mg). The mixture was stirred at 70° C. for 3 hours and concentrated to dryness. The residue was purified by flash chromatography, eluting with 20% ethyl acetate in heptane, to provide the title compound. MS (ESI) m/e 751.04 (M+H)⁺.

### 1.14.7. tert-butyl 3-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)-6-(1,2,3,4-tetrahydroquinolin-7-yl)picolinate

To a solution of 7-(4,4,5,5-tetramethyl-1,3,2-dioxaborolan-2-yl)-1,2,3,4-tetrahydroquinoline (172 mg) in dioxane (10 mL) and water (5 mL) was added Example 1.14.6 (500 mg), (Ph₃P)₂PdCl₂ (45.6 mg) and CsF (296 mg). The mixture was stirred at 120° C. for 30 minutes under microwave conditions (Biotage Initiator), diluted with ethyl acetate (200 mL) and washed with water and brine. The organic layer was dried over Na₂SO₄, filtered, and concentrated. The residue was purified by flash chromatography, eluting with 20% ethyl acetate in dichloromethane, to provide the title compound. MS (ESI) m/e 848.09 (M+H)⁺.

### 1.14.8. tert-butyl 6-(1-(benzo[d]thiazol-2-ylcarbamoyl)-1,2,3,4-tetrahydroquinolin-7-yl)-3-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)picolinate

To a suspension of bis(2,5-dioxopyrrolidin-1-yl) carbonate (63 mg) in acetonitrile (10 mL) was added benzo[d]thiazol-2-amine (37.2 mg). The mixture was stirred for 1 hour. A solution of Example 1.14.7 (210 mg) in acetonitrile (2 mL) was added, and the suspension was vigorously stirred overnight, diluted with ethyl acetate, and washed with water and brine. The organic layer was dried over Na₂SO₄, filtered, and concentrated to provide the title compound. MS (ESI) m/e 1024.50 (M+H)⁺.

### 1.14.9. 6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid

To a solution of Example 1.14.8 (230 mg) in tetrahydrofuran (10 mL) was added tetrabutyl ammonium fluoride (TBAF 10 mL, 1M in tetrahydrofuran). The mixture was stirred at room temperature overnight, diluted with ethyl acetate, and washed with water and brine. The organic layer was dried over Na₂SO₄, filtered, and concentrated. The residue was dissolved in dichloromethane (5 mL) and treated with trifluoroacetic acid (5 mL) overnight. The mixture was concentrated, and the residue was purified by reverse HPLC (Gilson), eluting with 10-85% acetonitrile in 0.1% TFA/water to provide the title compound. ¹H NMR (400 MHz, dimethyl sulfoxide-d₆) δ ppm 8.40 (d, 3H), 8.00 (d, 1H), 7.90-7.72 (m, 3H), 7.46 (s, 1H), 7.40-7.32 (m, 1H), 7.28 (d, 1H), 7.24-7.17 (m, 1H), 3.95 (d, 3H), 3.88 (s, 16H), 3.56 (dt, 5H), 3.28 (s, 3H), 3.18-2.96 (m, 5H), 2.82 (t, 2H), 2.21 (s, 3H), 1.93 (p, 2H), 1.43 (s, 2H), 1.30 (q, 5H), 1.21-0.97 (m, 7H), 0.86 (s, 6H) MS (ESI) m/e 804.3 (M+H)⁺.

### 1.15. Synthesis of 6-[8-(1,3-benzothiazol-2-ylcarbamoyl)naphthalen-2-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid (Compound W3.15)

### 1.15.1. 7-(6-(tert-butoxycarbonyl)-5-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)pyridin-2-yl)-1-naphthoic acid

To a solution of methyl 7-(4,4,5,5-tetramethyl-1,3,2-dioxaborolan-2-yl)-1-naphthoate (208 mg) in dioxane (10 mL) and water (5 mL) was added Example 1.14.6 (500 mg), (Ph₃P)₂PdCl₂ (45.6 mg) and CsF (296 mg). The mixture was stirred at 120° C. for 30 minutes under microwave conditions (Biotage Initiator), diluted with ethyl acetate and washed with water and brine. The organic layer was dried over Na₂SO₄, filtered, and concentrated. The residue was purified by flash chromatography, eluting with 20% ethyl acetate in dichloromethane, to give the ester intermediate. The ester was dissolved in a mixture of tetrahydrofuran (10 mL), methanol (5 mL) and H₂O (5 mL) and treated with lithium hydroxide monohydrate (200 mg). The mixture was stirred at room temperature for 4 hours, acidified with 1N aqueous HCl solution and diluted with ethyl acetate (300 mL). After washing with water ad brine, the organic layer was dried over Na₂SO₄. After filtration, evaporation of the solvent afforded the title compound. MS (ESI) m/e 888.20 (M+H)⁺.

### 1.15.2. 6-[8-(1,3-benzothiazol-2-ylcarbamoyl)naphthalen-2-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid

To a solution of Example 1.15.1 (500 mg) in dichloromethane (10 mL) was added benzo[d]thiazol-2-amine (85 mg), 1-ethyl-3-[3-(dimethylamino)propyl]-carbodiimide hydrochloride (216 mg) and 4-(dimethylamino)pyridine (138 mg). The mixture was stirred at room temperature overnight, diluted with ethyl acetate, and washed with water and brine. The organic layer was then dried over Na₂SO₄, filtered, and concentrated to dryness. The residue was dissolved in tetrahydrofuran (10 mL) and treated with tetrabutyl ammonium fluoride (10 mL, 1M in tetrahydrofuran) overnight. The reaction mixture was diluted with ethyl acetate and washed with water and brine. The organic layer was dried over Na₂SO₄, filtered, and concentrated to dryness. The residue was dissolved in dichloromethane (5 mL) and treated with trifluoroacetic acid (5 mL) overnight. The mixture was then concentrated and the residue was purified by reverse HPLC (Gilson), eluting with 10-85% acetonitrile in 0.1% TFA in water, to give the title compound. ¹H NMR (400 MHz, dimethyl sulfoxide-d₆) δ ppm 13.11 (s, 1H), 9.00 (s, 1H), 8.60-8.29 (m, 3H), 8.26-8.13 (m, 3H), 8.03 (ddd, 2H), 7.92 (d, 1H), 7.80 (d, 1H), 7.74-7.62 (m, 1H), 7.51-7.42 (m, 2H), 7.36 (td, 1H), 3.88 (s, 2H), 3.61-3.52 (m, 2H), 3.27 (s, 3H), 3.17-2.95 (m, 4H), 2.22 (s, 3H), 1.43 (s, 2H), 1.30 (q, 4H), 1.23-0.96 (m, 6H), 0.86 (s, 6H). MS (ESI) m/e 799.2 (M+H)⁺.

### 1.16. Synthesis of 6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.1^{3,7}]dec-1-yl}methyl)-5-methyl-1H-pyrazol-4-yl]pyridine-2-carboxylic acid (Compound W3.16)

### 1.16.1. methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.4.4 (15 g) in dimethyl sulfoxide (100 mL) was added N,N-diisopropylethylamine(12 mL). The mixture was stirred at 50° C. for 24 hours. The mixture was diluted with ethyl acetate (500 mL), washed with water and brine, and dried over Na₂SO₄. After filtration and evaporation of the solvent, the crude material was purified via silica gel column chromatography, eluting with 20% ethyl acetate in hexane, to give the title compound. MS (ESI) m/e 448.4 (M+H)⁺.

### 1.16.2. methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.16.1 (2.25 g) and [1,1′-bis(diphenylphosphino)ferrocene]dichloropalladium(11) (205 mg) in acetonitrile (30 mL) was added triethylamine (3 mL) and pinacolborane (2 mL). The mixture was stirred at reflux for 3 hours. The mixture was diluted with ethyl acetate (200 mL), washed with water and brine, and dried over Na₂SO₄. Filtration, evaporation of the solvent, and silica gel chromatography (eluting with 20% ethyl acetate in hexane) gave the title compound. MS (ESI) m/e 495.4 (M+H)⁺.

### 1.163. methyl 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.16.2 (4.94 g) in tetrahydrofuran (60 mL) and water (20 mL) was added Example 1.4.2 (5.57 g), 1,3,5,7-tetramethyl-8-tetradecyl-2,4,6-trioxa-8-phosphaadamantane (412 mg), tris(dibenzylideneacetone)dipalladium(0) (457 mg), and K₃PO₄ (11 g). The mixture was stirred at reflux overnight. The reaction mixture was diluted with ethyl acetate (500 mL), washed with water and brine, and dried over Na₂SO₄. After filtration and evaporation of the solvent, the crude material was purified via column chromatography, eluting with 20% ethyl acetate in heptane, to give the title compound. MS (ESI) m/e 784.4 (M+H)⁺.

### 1.16.4. 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

To a solution of Example 1.16.3 (10 g) in tetrahydrofuran (60 mL), methanol (30 mL) and water (30 mL), was added lithium hydroxide monohydrate (1.2 g). The mixture was stirred at room temperature for 24 hours. The reaction mixture was neutralized with 2% aqueous HCl and concentrated under vacuum. The residue was diluted with ethyl acetate (800 mL), washed with water and brine, and dried over Na₂SO₄. Filtration and evaporation of the solvent gave the title compound. MS (ESI) m/e 770.4 (M+H)⁺.

### 1.16.5. tert-butyl 6-(8-(benzo[d]thiazol-2-ylcarbamoyl)-3,4-dihydroisoquinolin-2(1H)-yl)-3-(1-((3-(2-((tert-butoxycarbonyl)amino)ethoxy)-5,7-dimethyladamantan-1-yl)methyl)-5-methyl-1H-pyrazol-4-yl)picolinate

To a solution of Example 1.16.4 (3.69 g) in N,N-dimethylformamide (20 mL) was added benzo[d]thiazol-2-amine(1.1 g), fluoro-N,N,N′,N′-tetramethylformamidinium hexafluorophosphate (1.9 g) and N,N diisopropylethylamine (1.86 g). The mixture was stirred at 60° C. for 3 hours. The reaction mixture was diluted with ethyl acetate (500 mL), washed with water and brine, and dried over Na₂SO₄. Filtration, evaporation of the solvent, and column purification (20% ethyl acetate in heptane) gave the title compound. MS (ESI) m/e 902.2(M+H)⁺.

### 1.16.6. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

Example 1.16.5 (2 g) was dissolved in 50% TFA in dichloromethane (20 mL) and stirred overnight. The solvents were removed under vacuum and the residue was loaded on a reverse-phase column and eluted with 20-80% acetonitrile in water (0.1% TFA) to give the title compound. MS (ESI) m/e 746.3 (M+H)⁺.

### 1.16.7. 6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.1^{3,7}]dec-1-yl}methyl)-5-methyl-1H-pyrazol-4-yl]pyridine-2-carboxylic acid

A solution of Example 1.16.6 (0.050 g), oxetan-3-one (5 mg) and sodium triacetoxyborohydride (0.018 g) was stirred together in dichloromethane (1 mL) at room temperature. After stirring for 1 hour, additional oxetan-3-one (5 mg) and sodium triacetoxyborohydride (0.018 g) were added and the reaction was stirred overnight. The reaction was concentrated, dissolved in a 1:1 mixture of dimethyl sulfoxide/methanol (2 mL) and purified by HPLC using a Gilson system (20-60% acetonitrile in water containing 0.1% v/v trifluoroacetic acid). The desired fractions were combined and freeze-dried to provide the title compound. ¹H NMR (400 MHz, dimethyl sulfoxide-d₆) δ ppm 12.95 (s, 1H), 9.26 (s, 2H), 8.12 (d, 1H), 7.88 (d, 1H), 7.71 (d, 1H), 7.63-7.50 (m, 3H), 7.50-7.41 (m, 2H), 7.38 (s, 1H), 7.05 (d, 1H), 5.05 (s, 2H), 4.79 (t, 2H), 4.68 (dd, 2H), 4.54-4.41 (m, 1H), 3.98 (t, 2H), 3.92 (s, 2H), 3.63 (t, 2H), 3.16-3.04 (m, 4H), 2.20 (s, 3H), 1.52 (s, 2H), 1.47-1.06 (m, 10H), 0.96 (s, 6H). MS (ESI) m/e 802.2 (M+H)⁺.

### 1.17. Synthesis of 6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid (Compound W3.17)

### 1.17.1. 4-iodo-1-((3-(2-methoxyethoxy)-5,7-dimethyladamantan-1-yl)methyl)-5-methyl-1H-pyrazole

Example 1.1.6 (3.00 g) was dissolved in 1,4-dioxane (40 mL), and sodium hydride (60% in mineral oil, 568 mg) was added. The solution was mixed at room temperature for 15 minutes, and methyl iodide (1.64 mL) was added. The solution was stirred at room temperature for three days, and then 0.01 M aqueous HCl solution (50 mL) was added. The solution was extracted with diethyl ether three times. The combined organic extracts were washed with brine and dried on anhydrous sodium sulfate. After filtration, the solvent was removed under reduced pressure and then under high vacuum to yield the title compound. MS (ESI) m/e 459 (M+H)⁺.

### 1.17.2. benzyl 4-oxopent-2-ynoate

Benzyl 4-hydroxypent-2-ynoate (40.5 g) and Dess-Martin Periodinane (93.0 g) in dichloromethane (500 mL) were stirred for 1 hour at 0° C. The solution was poured into diethyl ether (1 L), and the combined organics were washed three times with 1M aqueous NaOH and brine, dried over Na₂SO₄, filtered, and concentrated. The residue was chromatographed on silica gel using 5% ethyl acetate in heptanes to give the title compound.

### 1.17.3. (S)-benzyl 6-(3-((tert-butoxycarbonyl)amino)pyrrolidin-1-yl)-2-(2,2,2-trifluoroacetyl)-1,2,3,4-tetrahydroisoquinoline-8-carboxylate

A solution of 1-(2,2,2-trifluoroacetyl)piperidin-4-one (6.29 g), (S)-tert-butyl pyrrolidin-3-ylcarbamate (6.0 g), and p-toluenesulfonic acid monohydrate (0.613 g) in ethanol (80 mL) was stirred for 1 hour at room temperature. Example 1.17.2 (6.51 g) was then added and the reaction was stirred for 24 hours at room temperature, and heated to 45° C. for 3 days. The reaction was then cooled and poured into diethyl ether (600 mL). The resulting solution was washed twice with water and brine, dried over Na₂SO₄, filtered, and concentrated. The residue was chromatographed on silica gel using 5-50% ethyl acetate in heptanes to give the product.

### 1.17.4. (S)-benzyl 6-(3-((tert-butoxycarbonyl)amino)pyrrolidin-1-yl)-1,2,3,4-tetrahydroisoquinoline-8-carboxylate

A solution of Example 1.17.3 (3.1 g) and potassium carbonate (1.8 g) in a mixture of tetrahydrofuran (30 mL), methanol (10 mL), and water (25 mL) was stirred for 48 hours at 45° C. The reaction was then cooled and diluted with dichloromethane (300 mL). The layers were separated and the organic layer was dried over Na₂SO₄, filtered, and concentrated to give the title compound.

### 1.17.5. (S)-benzyl 2-(5-bromo-6-(tert-butoxycarbonyl)pyridin-2-yl)-6-(3-((tert-butoxycarbonyl)amino)pyrrolidin-1-yl)-1,2,3,4-tetrahydroisoquinoline-8-carboxylate

A solution of Example 1.17.4 (1.6 g), Example 1.4.4 (1.08 g), and triethylamine (0.59 mL) in N,N-dimethylformamide (10 mL) was heated to 50° C. for 24 hours. The reaction was cooled and poured into ethyl acetate (400 mL). The resulting solution was washed three times with water and brine, dried over Na₂SO₄, filtered, and concentrated. The residue was chromatographed on silica gel using 5-50% ethyl acetate in heptanes to give the product.

### 1.17.6. (S)-benzyl 2-(6-(tert-butoxycarbonyl)-5-(4,4,5,5-tetramethyl-1,3,2-dioxaborolan-2-yl)pyridin-2-yl)-6-(3-((tert-butoxycarbonyl)amino)pyrrolidin-1-yl)-1,2,3,4-tetrahydroisoquinoline-8-carboxylate

A solution of Example 1.17.5 (500 mg), 4,4,5,5-tetramethyl-1,3,2-dioxaborolane (136 mg), and triethylamine (0.200 mL) in acetonitrile (5 mL) was heated to 75° C. for 24 hours. The reaction was allowed to cool to room temperature and concentrated to dryness. The crude material was then purified via column chromatography, eluting with 5-50% ethyl acetate in heptanes, to give the title compound.

### 1.17.7. benzyl 2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-6-((S)-3-((tert-butoxycarbonyl)amino)pyrrolidin-1-yl)-1,2,3,4-tetrahydroisoquinoline-8-carboxylate

A solution of Example 1.17.6 (240 mg), Example 1.17.1 (146 mg). 1,3,5,7-tetramethyl-8-tetradecyl-2,4,6-trioxa-8-phosphaadamantane (13 mg), palladium (II)acetate (14.6 mg), and tripotassium phosphate (270 mg) in dioxane (7 mL) and water (3 mL) was heated to 70° C. for 24 hours. The reaction was allowed to cool to room temperature and was concentrated to dryness. The crude material was then purified via column chromatography, eluting with 5-25% ethyl acetate in heptanes, to give the title compound.

### 1.17.8. 2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-6-((S)-3-((tert-butoxycarbonyl)amino)pyrrolidin-1-yl)-1,2,3,4-tetrahydroisoquinoline-8-carboxylic acid

A solution of Example 1.17.7 (1.6 g) and lithium hydroxide monohydrate (5 mg) in a 3:1:1 mixture of tetrahydrofuran/methanol/water (10 mL) was stirred for 4 days. The reaction was acidified with 1M aqueous HCl solution and poured into ethyl acetate (150 mL). The resulting solution was washed with brine, dried over Na₂SO₄, filtered, and concentrated to give the title compound.

### 1.17.9. tert-butyl 6-(8-(benzo[d]thiazol-2-ylcarbamoyl)-6-((S)-3-((tert-butoxycarbonyl)amino)pyrrolidin-1-yl)-3,4-dihydroisoquinolin-2(1H)-yl)-3-(1-((3-(2-methoxyethoxy)-5,7-dimethyladamantan-1-yl)methyl)-5-methyl-1H-pyrazol-4-yl)picolinate

A solution of Example 1.17.8 (78 mg), benzo[d]thiazol-2-amine (16 mg), O-(7-azabenzotriazol-1-yl)-N,N,N′,N′-tetramethyluronium hexafluorophosphate (48 mg), and diisopropylethylamine (0.024 mL) in N,N-dimethylformamide (3 mL) was heated to 50° C. for 48 hours. The reaction was then cooled and poured into ethyl acetate (100 mL). The resulting solution was washed three times with water and brine, dried over Na₂SO₄, filtered, and concentrated. The residue was purified via column chromatography, eluting with 20-100% ethyl acetate in heptanes, to give the title compound.

### 1.17.10.6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid

Example 1.17.9 (40 mg) in dichloromethane (3 mL) was treated with trifluoroacetic acid (2 mL) overnight. The mixture was concentrated to provide the title compound as a TFA salt. MS (ESI) m/e 845.7 (M+H)⁺.

### 1.18. Synthesis of 6-[8-(1,3-benzothiazol-2-ylcarbamoyl)-3,4-dihydroisoquinolin-2(1H)-yl]-3-{(1-[(3,5-dimethyl-7-{2-[(2-sulfamoylethyl)amino]ethoxy}tricyclo[3.3.1.1^{3,7}]dec-1-yl)methyl]-5-methyl-1H-pyrazol-4-yl}pyridine-2-carboxylic acid (Compound W3.18)

### 1.18.1. 3-bromo-5,7-dimethyladamantanecarboxylic acid

Into a 50 mL round-bottomed flask at 0° C., was added bromine (16 mL). Iron powder (7 g) was added, and the reaction was stirred at 0° C. for 30 minutes. 3,5-Dimethyladamantane-1-carboxylic acid (12 g) was added. The mixture was warmed up to room temperature and stirred for 3 days. A mixture of ice and concentrated HCl was poured into the reaction mixture. The resulting suspension was treated twice with Na₂SO₃ (50 g in 200 mL water) and extracted three times with dichloromethane. The combined organics were washed with 1N aqueous HCl, dried over sodium sulfate, filtered, and concentrated to give the title compound.

### 1.18.2. 3-bromo-5,7-dimethyladamantanemethanol

To a solution of Example 1.18.1 (15.4 g) in tetrahydrofuran (200 mL) was added BH₃ (1M in tetrahydrofuran, 150 mL), and the mixture was stirred at room temperature overnight. The reaction mixture was then carefully quenched by adding methanol dropwise. The mixture was then concentrated under vacuum, and the residue was balanced between ethyl acetate (500 mL) and 2N aqueous HCl (100 mL). The aqueous layer was further extracted twice with ethyl acetate, and the combined organic extracts were washed with water and brine, dried over sodium sulfate, and filtered. Evaporation of the solvent gave the title compound.

### 1.18.3. 1-((3-bromo-5,7-dimethyltricyclo[3.3.1.1^{3,7}]dec-1-yl)methyl)-1H-pyrazole

To a solution of Example 1.18.2 (8.0 g) in toluene (60 mL) was added 1H-pyrazole (1.55 g) and cyanomethylenetributylphosphorane (2.0 g), and the mixture was stirred at 90° C. overnight. The reaction mixture was concentrated, and the residue was purified by silica gel column chromatography (10:1 heptane:ethyl acetate) to give the title compound. MS (ESI) m/e 324.2 (M+H)⁺.

### 1.18.4. 2-({[3,5-dimethyl-7-(1H-pyrazol-1-ylmethyl)tricyclo[3.3.1.1^{3,7}]dec-1-yl]oxy}ethanol

To a solution of Example 1.18.3 (4.0 g) in ethane-1,2-diol (12 mL) was added triethylamine (3 mL). The mixture was stirred at 150° C. under microwave conditions (Biotage Initiator) for 45 minutes. The mixture was poured into water (100 mL) and extracted three times with ethyl acetate. The combined organic extracts were washed with water and brine, dried over sodium sulfate, and filtered. Evaporation of the solvent gave a residue that was purified by silica gel chromatography, eluting with 20% ethyl acetate in heptane, followed by 5% methanol in dichloromethane, to give the title compound. MS (ESI) m/e 305.2 (M+H)⁺.

### 1.18.5. 2-({3,5-dimethyl-7-[(5-methyl-1H-pyrazol-1-yl)methyl]tricyclo[3.3.1.1^{3,7}]dec-1-yl}oxy)ethanol

To a cooled (−78° C.) solution of Example 1.18.4 (6.05 g) in tetrahydrofuran (100 mL) was added n-BuLi (40 mL, 2.5M in hexane), and the mixture was stirred at −78° C. for 1.5 hours. Iodomethane (10 mL) was added through a syringe, and the mixture was stirred at −78° C. for 3 hours. The reaction mixture was then quenched with aqueous NH₄Cl and extracted twice with ethyl acetate, and the combined organic extracts were washed with water and brine. After drying over sodium sulfate, the solution was filtered and concentrated, and the residue was purified by silica gel column chromatography, eluting with 5% methanol in dichloromethane, to give the title compound. MS (ESI) m/e 319.5 (M+H)⁺.

### 1.18.6. 1-({3,5-dimethyl-7-[2-(hydroxy)ethoxy]tricyclo[3.3.1.1^{3,7}]dec-1-yl}methyl)-4-iodo-5-methyl-1H-pyrazole

To a solution of Example 1.18.5 (3.5 g) in N,N-dimethylformamide (30 mL) was added N-iodosuccinimide (3.2 g), and the mixture was stirred at room temperature for 1.5 hours. The reaction mixture was diluted with ethyl acetate (600 mL) and washed with aqueous NaHSO₃, water and brine. The organic layer was dried over sodium sulfate, filtered and concentrated under reduced pressure. The residue was purified by silica gel chromatography, eluting with 20% ethyl acetate in dichloromethane, to give the title compound. MS (ESI) m/e 445.3 (M+H)⁺.

### 1.18.7. 1-((3-(2-((tert-butyldimethylsilyl)oxy)ethoxy)-5,7-dimethyladamantan-1-yl)methyl)-4-iodo-5-methyl-1H-pyrazole

Tert-butyldimethylsilyl trifluoromethanesulfonate (5.34 mL) was added to a solution of Example 1.18.6 (8.6 g) and 2,6-lutidine (3.16 mL) in dichloromethane (125 mL) at −40° C., and the reaction was allowed to warm to room temperature overnight. The mixture was concentrated, and the residue was purified by silica gel chromatography, eluting with 5-20% ethyl acetate in heptanes, to give the title compound. MS (ESI) m/e 523.4 (M+H)⁺.

### 1.18.8. 1-((3-(2-((tert-butyldimethylsilyl)oxy)ethoxy)-5,7-dimethyladamantan-1-yl)methyl)-5-methyl-4-(4,4,5,5-tetramethyl-1,3,2-dioxaborolan-2-yl)-1H-pyrazole

n-Butyllithium (8.42 mL, 2.5M in hexanes) was added to Example 1.18.7 (9.8 g) in 120 mL tetrahydrofuran at −78° C., and the reaction was stirred for 1 minute. Trimethyl borate (3.92 mL) was added, and the reaction stirred for 5 minutes. Pinacol (6.22 g) was added, and the reaction was allowed to warm to room temperature and was stirred 2 hours. The reaction was quenched with pH 7 buffer, and the mixture was poured into ether. The layers were separated, and the organic layer was concentrated under reduced pressure. The residue was purified by silica gel chromatography, eluting with 1-25% ethyl acetate in heptanes, to give the title compound.

### 1.18.9. 6-fluoro-3-bromopicolinic acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour. The resulting mixture was stirred for another 30 minutes, then warmed to 35° C. and stirred overnight. The reaction was cooled to room temperature, and then adjusted to pH 4 with aqueous NaH₂PO₄ solution. The resulting solution was extracted three times with dichloromethane, and the combined extracts were washed with brine, dried over sodium sulfate, filtered and concentrated to provide the title compound.

### 1.18.10. Tert-butyl 3-bromo-6-fluoropicolinate

Para-toluenesulfonyl chloride (27.6 g) was added to a solution of Example 1.18.9 (14.5 g) and pyridine (26.7 mL) in dichloromethane (100 mL) and tert-butanol (80 mL) at 0° C. The reaction was stirred for 15 minutes, and then warmed to room temperature, and stirred overnight. The solution was concentrated and partitioned between ethyl acetate and aqueous Na₂CO₃ solution. The layers were separated, and the aqueous layer extracted with ethyl acetate. The organic layers were combined, rinsed with aqueous Na₂CO₃ solution and brine, dried over sodium sulfate, filtered, and concentrated to provide the title compound.

### 1.18.11. methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.18.10 (15 g) in dimethyl sulfoxide (100 mL) was added N,N-diisopropylethylamine (12 mL), and the mixture was stirred at 50° C. for 24 hours. The mixture was then diluted with ethyl acetate (500 mL) and washed with water and brine. The organic layer was dried over sodium sulfate, filtered and concentrated under reduced pressure. The residue was purified by silica gel chromatography, eluting with 20% ethyl acetate in hexane, to give the title compound. MS (ESI) m/e 448.4 (M+H)⁺.

### 1.18.12. methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.18.11 (2.25 g) and [1,1′-bis(diphenylphosphino)ferrocene]dichloropalladium(II) (205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered and concentrated under reduced pressure. Purification of the residue by silica gel chromatography, eluting with 20% ethyl acetate in hexane, provided the title compound.

### 1.18.13. methyl 2-(6-(tert-butoxycarbonyl)-5-(1-((3-(2-hydroxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.18.12 (2.25 g) in tetrahydrofuran (30 mL) and water (10 mL) was added Example 1.18.6 (2.0 g), 1,3,5,7-tetramethyl-6-phenyl-2,4,8-trioxa-6-phosphaadamantane (329 mg), tris(dibenzylideneacetone)dipalladium(0) (206 mg) and potassium phosphate tribasic (4.78 g). The mixture was refluxed overnight, cooled and diluted with ethyl acetate (500 mL). The resulting mixture was washed with water and brine, and the organic layer was dried over sodium sulfate, filtered and concentrated. The residue was purified by flash chromatography, eluting with 20% ethyl acetate in heptanes followed by 5% methanol in dichloromethane, to provide the title compound.

### 1.18.14. methyl 2-(6-(tert-butoxycarbonyl)-5-(1-((3,5-dimethyl-7-(2-((methylsulfonyl)oxy)ethoxy)adamantan-1-yl)methyl)-5-methyl-1H-pyrazol-4-yl)pyridin-2-yl)-1,2,3,4-tetrahydroisoquinoline-8-carboxylate

To a cold solution of Example 1.18.13 (3.32 g) in dichloromethane (100 mL) in an ice-bath was sequentially added triethylamine (3 mL) and methanesulfonyl chloride (1.1 g). The reaction mixture was stirred at room temperature for 1.5 hours and diluted with ethyl acetate, and washed with water and brine. The organic layer was dried over sodium sulfate, filtered, and concentrated to provide the title compound.

### 1.18.15. methyl 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1,2,3,4-tetrahydroisoquinoline-8-carboxylate

To a solution of Example 1.18.14 (16.5 g) in N,N-dimethylformamide (120 mL) was added sodium azide (4.22 g). The mixture was heated at 80° C. for 3 hours, cooled, diluted with ethyl acetate and washed with water and brine. The organic layer was dried over sodium sulfate, filtered, and concentrated. The residue was purified by flash chromatography, eluting with 20% ethyl acetate in heptanes, to provide the title compound.

### 1.18.16. 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1,2,3,4-tetrahydroisoquinoline-8-carboxylic acid

To a solution of Example 1.18.15 (10 g) in a mixture of tetrahydrofuran (60 mL), methanol (30 mL) and water (30 mL) was added lithium hydroxide monohydrate (1.2 g). The mixture was stirred at room temperature overnight and neutralized with 2% aqueous HCl. The resulting mixture was concentrated, and the residue was dissolved in ethyl acetate (800 mL), and washed with brine. The organic layer was dried over sodium sulfate, filtered, and concentrated to provide the title compound.

### 1.18.17. tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

A mixture of Example 1.18.16 (10 g), benzo[d]thiazol-2-amine (3.24 g), fluoro-N,N,N′,N′-tetramethylformamidinium hexafluorophosphate (5.69 g) and N,N-diisopropylethylamine (5.57 g) in N,N-dimethylformamide (20 mL) was heated at 60° C. for 3 hours, cooled and diluted with ethyl acetate. The resulting mixture was washed with water and brine. The organic layer was dried over sodium sulfate, filtered, and concentrated. The residue was purified by flash chromatography, eluting with 20% ethyl acetate in dichloromethane to give the title compound.

### 1.18.18. tert-butyl 3-(1-(((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

To a solution of Example 1.18.17 (2.0 g) in tetrahydrofuran (30 mL) was added Pd/C (10%, 200 mg). The mixture was stirred under a hydrogen atmosphere overnight. The insoluble material was filtered off and the filtrate was concentrated to provide the title compound.

### 1.18.19. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

Example 1.18.18 (200 mg) in dichloromethane (2.5 mL) was treated with trifluoroacetic acid (2.5 mL) overnight. The reaction mixture was concentrated, and the residue was purified by reverse phase chromatography (C18 column), eluting with 20-60% acetonitrile in water containing 0.1% v/v trifluoroacetic acid, to provide the title compound. MS (ESI) m/e 746.2 (M+H)⁺.

### 1.18.20. 6-[8-(1,3-benzothiazol-2-ylcarbamoyl)-3,4-dihydroisoquinolin-2(1H)-yl]-3-{1-[(3,5-dimethyl-7-{2-[(2-sulfamoylethyl)amino]ethoxy}tricyclo[3.3.1.1^{3,7}]dec-1-yl)methyl]-5-methyl-1H-pyrazol-4-yl}pyridine-2-carboxylic acid

A mixture of Example 1.18.19 (18 mg) and ethenesulfonamide (5.2 mg) in N,N-dimethylformamide (1 mL) and water (0.3 mL) was stirred for one week. The mixture was purified by reverse phase chromatography (C18 column), eluting with 20-60% acetonitrile in water containing 0.1% v/v trifluoroacetic acid, to provide the title compound. ¹H NMR (500 MHz, dimethyl sulfoxide-d₆) δ ppm 8.03 (d, 1H), 7.79 (d, 1H), 7.61 (d, 1H), 7.45-7.50 (m, 1H), 7.41-7.44 (m, 1H), 7.33-7.39 (m, 3H), 7.23 (s, 1H), 6.73 (d, 1H), 4.87 (s, 2H), 3.89 (t, 2H), 3.79 (s, 2H), 3.12-3.20 (m, 2H), 2.99 (t, 2H), 2.85 (s, 2H), 2.09 (s, 3H), 1.32 (dd, 4H), 1.08-1.19 (m, 5H), 1.04 (d, 4H), 0.86 (s, 6H). MS (ESI) m/e 853.2(M+H)⁺.

### 1.19 Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid

### 1.19.1 6,7-dihydro-4H-thieno[3,2-c]pyridine-3,5-dicarboxylic acid 5-tert-butyl ester 3-methyl ester

Tert-butyl 3-bromo-6,7-dihydrothieno[3,2-c]pyridine-5(4H)-carboxylate (1000 mg) and dichloro[1,1′-bis(diphenylphosphino)ferrocene]palladium(II) (69 mg) were placed in a 50 mL pressure bottle, and methanol (20 mL) was added, followed by trimethylamine (636 mg). The solution was degassed and flushed with argon three times. The solution was then degassed and flushed with carbon monoxide and heated to 100° C. for 18 hours under 60 psi of carbon monoxide. The solvent was removed under reduced pressure, and the residue was purified by flash column chromatography on silica gel, eluting with 50% ethyl acetate in heptanes. The solvent was removed under reduced pressure to yield the title compound.

### 1.19.2 4,5,6,7-tetrahydro-thieno[3,2-c]pyridine-3-carboxylic acid methyl ester

Example 1.19.1 (940 mg) was dissolved in dichloromethane (12 mL). Trifluoroacetic acid (2220 mg) was added, and the solution was stirred for three hours. The solvent was removed under reduced pressure to yield the title compound as the trifluoroacetic acid salt, which was used without further purification.

### 1.19.3 5-(5-bromo-6-tert-butoxycarbonyl-pyridin-2-yl)-4,5,6,7-tetrahydro-thieno[3,2-c]pyridine-3-carboxylic acid methyl ester

The title compound was prepared by substituting Example 1.19.2 for ethyl 5,6,7,8-tetrahydroimidazo[1,5-a]pyrazine-1-carboxylate hydrochloride in Example 1.4.5. MS (ESI) m/e 452, 450 (M+H)⁺.

### 1.19.4 5-[6-tert-butoxycarbonyl-5-(4,4,5,5-tetramethyl-[1,3,2]dioxaborolan-2-yl)-pyridin-2-yl]-4,5,6,7-tetrahydro-thieno[3,2-c]pyridine-3-carboxylic acid methyl ester

The title compound was prepared by substituting Example 1.19.3 for Example 1.1.9 in Example 1.1.10. MS (ESI) m/e 500 (M+H)⁺, 531 (M+CH₃OH−H)⁻.

### 1.19.5 5-(6-tert-butoxycarbonyl-5-{1-[5-(2-tert-butoxycarbonylamino-ethoxy)-3,7-dimethyl-adamantan-1-ylmethyl]-5-methyl-1H-pyrazol-4-yl}-pyridin-2-yl)-4,5,6,7-tetrahydro-thieno[3,2-c]pyridine-3-carboxylic acid methyl ester

The title compound was prepared by substituting Example 1.19.4 for Example 1.4.6 in Example 1.4.7.

### 1.19.6 5-(6-tert-butoxycarbonyl-5-{1-[5-(2-tert-butoxycarbonylamino-ethoxy)-3,7-dimethyl-adamantan-1-ylmethyl]-5-methyl-1H-pyrazol-4-yl}-pyridin-2-yl)-4,5,6,7-tetrahydro-thieno[3,2-c]pyridine-3-carboxylic acid

The title compound was prepared by substituting Example 1.19.5 for Example 1.4.7 in Example 1.4.8. MS (ESI) m/e 776 (M+H)⁺, 774 (M−H)⁻.

### 1.19.7 6-[3-(benzothiazol-2-ylcarbamoyl)-6,7-dihydro-4H-thieno[3,2-c]pyridin-5-yl]-3-{(1-[5-(2-tert-butoxycarbonylamino-ethoxy)-3,7-dimethyl-adamantan-1-ylmethyl]-5-methyl-1H-pyrazol-4-yl}-pyridine-2-carboxylic acid tert-butyl ester

The title compound was prepared by substituting Example 1.19.6 for Example 1.4.8 in Example 1.4.9. MS (ESI) m/e 892 (M+H)⁺, 890 (M−H)⁻.

### 1.19.8 3-(1-{[3-(2-aminoethoxy)-5,7-dimethyltricyclo[3.3.1.1^{3,7}]dec-1-yl]methyl}-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid

The title compound was prepared by substituting Example 1.19.7 for Example 1.1.13 in Example 1.1.14. ¹H NMR (400 MHz, dimethyl sulfoxide-d₆) δ ppm 8.11 (bs, 1H), 8.00 (d, 1H), 7.77 (d, 1H), 7.68 (bs, 3H), 7.53 (d, 1H), 7.47 (t, 1H), 7.36-7.31 (m, 2H), 7.14 (d, 1H), 4.71 (s, 2H), 3.99 (t, 2H), 3.85 (s, 2H), 3.52 (m, 2H), 3.00 (t, 2H), 2.91 (q, 2H), 2.13 (s, 3H), 1.44 (s, 2H), 1.31 (q, 4H), 1.16 (m, 4H), 1.05 (q, 2H), 0.88 (s, 6H). MS (ESI) m/e 752 (M+H)⁺, 750 (M−H)⁻.

### 1.20 Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid

### 1.20.1 7-(5-bromo-6-tert-butoxycarbonyl-pyridin-2-yl)-3-trifluoromethyl-5,6,7,8-tetrahydro-imidazo[1,5-a]pyrazine-1-carboxylic acid methyl ester

The title compound was prepared by substituting methyl 3-(trifluoromethyl)-5,6,7,8-tetrahydroimidazo[1,5-a]pyrazine-1-carboxylate for ethyl 5,6,7,8-tetrahydroimidazo[1,5-a]pyrazine-1-carboxylate hydrochloride in Example 1.4.5. MS (ESI) m/e 449 (M-tBu+H)⁺, 503 (M−H)⁻.

### 1.20.2 7-[6-tert-butoxycarbonyl-5-(4,4,5,5-tetramethyl-[1,3,2]dioxaborolan-2-yl)-pyridin-2-yl]-3-trifluoromethyl-5,6,7,8-tetrahydro-imidazo[1,5-a]pyrazine-1-carboxylic acid methyl ester

The title compound was prepared by substituting Example 1.20.1 for Example 1.1.9 in Example 1.1.10. MS (ESI) m/e 553 (M+H)⁺.

### 1.20.3 di-tert-butyl [2-({3-[(4-iodo-5-methyl-1H-pyrazol-1-yl)methyl]-5,7-dimethyltricyclo[3.3.1.1^{3,7}]decan-1-yl}oxy)ethyl]-2-imidodicarbonate

Example 1.1.6 (5.000 g) was dissolved in dichloromethane (50 mL). Triethylamine (1.543 g) was added, and the solution was cooled on an ice bath. Methanesulfonyl chloride (1.691 g) was added dropwise. The solution was allowed to warm to room temperature and stir for 30 minutes. Saturated aqueous sodium bicarbonate solution (50 mL) was added. The layers were separated, and the organic layer was washed with brine (50 mL). The aqueous portions were then combined and back extracted with dichloromethane (50 mL). The organic portions were combined, dried over anhydrous sodium sulfate, filtered, and concentrated. The residue was dissolved in acetonitrile (50 mL). Di-tert-butyl iminodicarboxylate (2.689 g) and cesium carbonate (7.332 g) were added, and the solution was refluxed for 16 hours. The solution was cooled and added to diethyl ether (100 mL) and water (100 mL). The layers were separated. The organic portion was washed with brine (50 mL). The aqueous portions were then combined and back extracted with diethyl ether (100 mL). The organic portions were combined, dried over anhydrous sodium sulfate, filtered, and concentrated under reduced pressure. The material was purified by flash column chromatography on silica gel, eluting with 20% ethyl acetate in heptanes. The solvent was evaporated under reduced pressure to provide the title compound. MS (ESI) m/e 666 (M+Na)⁺.

### 1.20.4 methyl 7-(6-(tert-butoxycarbonyl)-5-(1-((3-(2-(di-(tert-butoxycarbonyl)amino)ethoxy)-5,7-dimethyladamantan-1-yl)methyl)-5-methyl-1H-pyrazol-4-yl)pyridin-2-yl)-3-(trifluoromethyl)-5,6,7,8-tetrahydroimidazo[1,5-a]pyrazine-1-carboxylate

The title compound was prepared by substituting Example 1.20.2 for Example 1.4.6 and Example 1.20.3 for Example 1.4.2 in Example 1.4.7. MS (ESI) m/e 964 (M+Na)⁺. 940 (M−H)⁻.

### 1.20.5 7-(6-(tert-butoxycarbonyl)-5-(1-((3-(2-(di-(tert-butoxycarbonyl)amino)ethoxy)-5,7-dimethyladamantan-1-yl)methyl)-5-methyl-1H-pyrazol-4-yl)pyridin-2-yl)-3-(trifluoromethyl)-5,6,7,8-tetrahydroimidazo[1,5-a]pyrazine-1-carboxylic acid

The title compound was prepared by substituting Example 1.20.4 for Example 1.4.7 in Example 1.4.8. MS (ESI) m/e 828 (M+H)⁺, 826 (M−H)⁻.

### 1.20.6 tert-butyl 6-(1-(benzo[d]thiazol-2-ylcarbamoyl)-3-(trifluoromethyl)-5,6-dihydroimidazo[1,5-a]pyrazin-7(8H)-yl)-3-(1-((3-(2-(di-(tert-butoxycarbonyl)amino)ethoxy)-5,7-dimethyladamantan-1-yl)methyl)-5-methyl-1H-pyrazol-4-yl)picolinate

The title compound was prepared by substituting Example 1.20.5 for Example 1.4.8 in Example 1.4.9. MS (ESI) m/e 1058 (M−H)⁻.

### 1.20.7 3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid

The title compound was prepared by substituting Example 1.20.6 for Example 1.1.13 in Example 1.1.14. ¹H NMR (400 MHz, dimethyl sulfoxide-d₆) δ ppm 11.99 (bs, 1H), 8.00 (d, 1H), 7.79 (d, 1H), 7.66 (bs, 3H), 7.61 (d, 1H), 7.47 (t, 1H), 7.35 (t, 2H), 7.19 (d, 1H), 5.20 (s, 2H), 4.37 (t, 2H), 4.16 (t, 2H), 3.86 (s, 2H), 3.51 (t, 2H), 2.91 (q, 2H), 2.14 (s, 3H), 1.44 (s, 2H), 1.36-1.24 (m, 4H), 1.19-1.02 (m, 6H), 0.88 (s, 6H). MS (ESI) m/e 804 (M+H)⁺, 802 (M−H)⁻.

### 1.21 Synthesis of 6-[8-(1,3-benzothiazol-2-ylcarbamoyl)-6-{methyl[2-(methylamino)ethyl]amino}-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid

### 1.21.1 methyl 3-bromo-5-(bromomethyl)benzoate

AIBN (2,2′-azobis(2-methylpropionitrile)) (1.79 g) was added to methyl 3-bromo-5-methylbenzoate (50 g) and N-bromosuccinimide (44.7 g) in 350 mL acetonitrile, and the mixture was refluxed overnight. An additional 11 g of N-bromosuccinimide and 0.5 g of AIBN (2,2′-azobis(2-methylpropionitrile)) was added, and the refluxing was continued for 3 hours. The mixture was concentrated, and then taken up in 500 mL ether, and stirred for 30 minutes. The mixture was then filtered, and the resulting solution was concentrated. The crude product was chromatographed on silica gel using 10% ethyl acetate in heptane to give the title compound.

### 1.21.2 methyl 3-bromo-5-(cyanomethyl)benzoate

Tetrabutylammonium cyanide (50 g) was added to Example 1.21.1 (67.1 g) in 300 mL acetonitrile, and the mixture was heated to 70° C. overnight. The mixture was cooled, poured into diethyl ether, and rinsed with water and brine. The mixture was concentrated and chromatographed on silica gel using 2-20% ethyl acetate in heptane to give the title compound.

### 1.21.3 methyl 3-(2-aminoethyl)-5-bromobenzoate

Borane-tetrahydrofuran complex (126 mL, 1M solution) was added to a solution of Example 1.21.2 (16 g) in 200 mL tetrahydrofuran, and the mixture was stirred overnight. The reaction was carefully quenched with methanol (50 mL), and then concentrated to 50 mL volume. The mixture was then taken up in 120 mL methanol/120 mL 4M HCl/120 mL dioxane, and stirred overnight. The organics were removed by evaporation under reduced pressure, and the residue was extracted with diethyl ether (2×). The organic extracts were discarded. The aqueous layer was basified with solid K₂CO₃, and then extracted with ethyl acetate, and dichloromethane (2×). The extracts were combined, dried over Na₂SO₄, filtered and concentrated to give the title compound.

### 1.21.4 methyl 3-bromo-5-(2-(2,2,2-trifluoroacetamido)ethyl)benzoate

Trifluoroacetic anhydride (9.52 mL) was added dropwise to a mixture of Example 1.21.3 (14.5 g) and triethylamine (11.74 mL) in 200 mL dichloromethane at 0° C. Upon addition, the mixture was allowed to warm to room temperature and was stirred for three days. The mixture was poured into diethyl ether, and washed with NaHCO₃ solution and brine. The mixture was concentrated and chromatographed on silica gel using 5-30% ethyl acetate in heptanes to give the title compound.

### 1.21.5 methyl 6-bromo-2-(2,2,2-trifluoroacetyl)-1,2,3,4-tetrahydroisoquinoline-8-carboxylate

Sulfuric acid was added to Example 1.21.4 (10 g) until it went into solution (40 mL), at which time paraformaldehyde (4.24 g) was added, and the mixture was stirred for 2 hours. The solution was then poured onto 400 mL ice, and stirred 10 minutes. It was then extracted with ethyl acetate (3×), and the combined extracts were washed with NaHCO₃ solution and brine, and then concentrated The crude product was chromatographed on silica gel using 2-15% ethyl acetate in heptanes to give the title compound.

### 1.21.6 methyl 6-((2-((tert-butoxycarbonyl)(methyl)amino)ethyl)(methyl)amino)-2-(2,2,2-trifluoroacetyl)-1,2,3,4-tetrahydroisoquinoline-8-carboxylate

Example 1.21.5 (2.25 g), tert-butyl methyl(2-(methylamino)ethyl)carbamate (1.27 g), palladium (II) acetate (0.083 g), 4,5-bis(diphenylphosphino)-9,9-dimethylxanthene (0.213 g) and cesium carbonate (4.00 g) were stirred in 40 mL dioxane at 80° C. overnight. The mixture was concentrated and chromatographed on silica gel using 5-50% ethyl acetate in heptanes to give the title compound.

### 1.21.7 methyl 2-(5-bromo-6-(tert-butoxycarbonyl)pyridin-2-yl)-6-((2-((tert-butoxycarbonyl)(methyl)amino)ethyl)(methyl)amino)-1,2,3,4-tetrahydroisoquinoline-8-carboxylate

Example 1.21.6 (3 g) and potassium carbonate (2.63 g) were stirred in 30 mL tetrahydrofuran, 20 mL methanol, and 25 mL water overnight. The mixture was concentrated and 60 mL N,N-dimethylformamide was added. To this was then added Example 1.4.4 (1.08 g) and triethylamine (0.6 mL), and the reaction was stirred at 50° C. overnight. The mixture was cooled to room temperature and poured into ethyl acetate (200 mL). The solution was washed with water (3×) and brine, then dried over Na₂SO₄, filtered, and concentrated. The residue was chromatographed on silica gel using 5-50% ethyl acetate in heptanes to give the title compound. MS (ESI) m/e 635 (M+H)⁺.

### 1.21.8 methyl 6-((2-((tert-butoxycarbonyl)(methyl)amino)ethyl)(methyl)amino)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

The title compound was prepared by substituting Example 1.21.7 for Example 1.1.9 in Example 1.1.10.

### 1.21.9 methyl 6-((2-((tert-butoxycarbonyl)(methyl)amino)ethyl)(methyl)amino)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared by substituting Example 1.21.8 for Example 1.5.11 and Example 1.17.1 for Example 1.5.10 in Example 1.5.12. MS (ESI) m/e 885.6 (M+H)⁺.

### 1.21.10 6-((2-((tert-butoxycarbonyl)(methyl)amino)ethyl)(methyl)amino)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared by substituting Example 1.21.9 for Example 1.4.7 in Example 1.4.8.

### 1.21.11 tert-butyl 6-(8-(benzo[d]thiazol-2-ylcarbamoyl)-6-((2-((tert-butoxycarbonyl)(methyl)amino)ethyl)(methyl)amino)-3,4-dihydroisoquinolin-2(1H)-yl)-3-(1-((3-(2-methoxyethoxy)-5,7-dimethyladamantan-1-yl)methyl)-5-methyl-1H-pyrazol-4-yl)picolinate

The title compound was prepared by substituting Example 1.21.10 for Example 1.4.8 in Example 1.4.9. MS (ESI) m/e 1003.6 (M+H)⁺.

### 1.21.12 6-[8-(1,3-benzothiazol-2-ylcarbamoyl)-6-{methyl[2-(methylamino)ethyl]amino}-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid

Example 1.21.11 (40 mg) was stirred in 2 mL trifluoroacetic acid and 3 mL dichloromethane overnight. After evaporation of the solvent, the residue was purified on an HPLC (Gilson system, eluting with 10-85% acetonitrile in 0.1% trifluoroacetic acid in water) to give the title compound. ¹H NMR (400 MHz, dimethyl sulfoxide-d₆) δ ppm 12.75 (bs, 1H), 12.50 (br s, 1H), 8.40 (m, 2H), 8.01 (d, 1H), 7.76 (d, 1H), 7.45 (m, 2H), 7.32 (t, 1H), 7.24 (s, 1H), 6.99 (d, 1H), 6.86 (d, 1H), 6.78 (d, 1H), 4.72 (m, 2H), 3.98 (m, 2H), 3.80 (m, 4H), 3.76 (s, 2H), 3.55 (m, 2H), 3.29 (d, 3H), 3.20 (s, 3H), 3.15 (m, 2H), 2.90 (s, 3H), 2.58 (t, 2H), 2.05 (s, 3H), 1.30 (s, 2H), 1.21 (m, 4H), 1.08 (m, 4H), 0.98 (m, 2H), 0.85 (s, 6H). MS (ESI) m/e 847.5 (M+H)⁺.

### 1.22 Synthesis of 6-[8-(1,3-benzothiazol-2-ylcarbamoyl)-6-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.22.1 methyl 6-(4,4,5,5-tetramethyl-1,3,2-dioxaborolan-2-yl)-2-(2,2,2-trifluoroacetyl)-1,2,3,4-tetrahydroisoquinoline-8-carboxylate

A mixture of Example 1.21.5 (4.5 g), 4,4,4,4′,4′,5,5,5′,5′-octamethyl-2,2′-bi(1,3,2-dioxaborolane) (3.75 g), [1,1′-bis(diphenylphosphino)ferrocene]dichloropalladium(II) dichloromethane (0.4 g), and potassium acetate (3.62 g) was stirred in 60 mL dioxane at 70° C. for 24 hours. The mixture was then diluted with ethyl acetate, and rinsed with water and brine. The mixture was concentrated and chromatographed on silica gel using 5-50% ethyl acetate in heptanes to give the title compound.

### 1.22.2 methyl 6-hydroxy-2-(2,2-trifluoroacetyl)-1,2,3,4-tetrahydroisoquinoline-8-carboxylate

Hydrogen peroxide (30%, 1.1 mL) was added to a mixture of Example 1.22.1 (4 g) and 1M aqueous NaOH solution (9.86 mL) in 40 mL tetrahydrofuran and 40 mL water, and the mixture was stirred for 90 minutes. The solution was acidified with concentrated HCl, and extracted twice with ethyl acetate. The combined extracts were washed with brine. The mixture was then concentrated and chromatographed on silica gel using 5-50% ethyl acetate in heptanes to give the title compound. MS (ESI) m/e 304.2 (M+H)⁺.

### 1.22.3 methyl 6-methoxy-2-(2,2,2-trifluoroacetyl)-1,2,3,4-tetrahydroisoquinoline-8-carboxylate

Trimethylsilyldiazomethane (2.6 mL, 2M solution in diethyl ether) was added to Example 1.22.2 (800 mg) in 10 mL methanol, and the reaction was stirred for 24 hours. The mixture was then concentrated and chromatographed on silica gel using 5-25% ethyl acetate in heptanes to give the title compound. MS (ESI) m/e 318.2 (M+H)⁺.

### 1.22.4 methyl 2-(5-bromo-6-(tert-butoxycarbonyl)pyridin-2-yl)-6-methoxy-1,2,3,4-tetrahydroisoquinoline-8-carboxylate

The title compound was prepared by substituting Example 1.22.3 for Example 1.21.6 in Example 1.21.7. MS (ESI) m/e 479.1 (M+H)⁺.

### 1.22.5 methyl 2-(6-(tert-butoxycarbonyl)-5-(4,4,5,5-tetramethyl-1,3,2-dioxaborolan-2-yl)pyridin-2-yl)-6-methoxy-1,2,3,4-tetrahydroisoquinoline-8-carboxylate

The title compound was prepared by substituting Example 1.22.4 for Example 1.1.9 in Example 1.1.10. MS (ESI) m/e 525.1 (M+H)⁺.

### 1.22.6 methyl 2-(6-(tert-butoxycarbonyl)-5-(1-((-(2-((tert-butoxycarbonyl)(methyl)amino)ethoxy)-5,7-dimethyladamantan-1-yl)methyl)-5-methyl-1H-pyrazol-4-yl)pyridin-2-yl)-6-methoxy-1,2,3,4-tetrahydroisoquinoline-8-carboxylate

The title compound was prepared by substituting Example 1.22.5 for Example 1.5.11 and Example 1.1.9 for Example 1.5.10 in Example 1.5.12. MS (ESI) m/e 829.6 (M+H)⁺.

### 1.22.7 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-6-methoxy-1,2,3,4-tetrahydroisoquinoline-8-carboxylic acid

The title compound was prepared by substituting Example 1.22.6 for Example 1.4.7 in Example 1.4.8. MS (ESI) m/e 814.6 (M+H)⁺.

### 1.22.8 tert-butyl 6-(8-(benzo[d]thiazol-2-ylcarbamoyl)-6-methoxy-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.22.7 for Example 1.4.8 in Example 1.4.9. MS (ESI) m/e 946.5 (M+H)⁺.

### 1.22.9 6-[8-(1,3-benzothiazol-2-ylcarbamoyl)-6-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared by substituting Example 1.22.8 for Example 1.21.11 in Example 1.21.12. ¹H NMR (400 MHz, dimethyl sulfoxide-d₆) δ ppm 12.75 (bs, 1H), 12.50 (br s, 1H), 8.21 (m, 2H), 8.01 (d, 1H), 7.76 (d, 1H), 7.44 (m, 2H), 7.32 (t, 1H), 7.25 (s, 1H), 7.20 (d, 1H), 6.99 (d, 1H), 6.90 (d, 1H), 4.72 (m, 2H), 3.80 (m, 4H), 3.55 (s, 3H), 3.50 (d, 3H), 2.98 (m, 4H), 2.51 (t, 2H), 2.05 (s, 3H), 1.35 (s, 2H), 1.26 (m, 4H), 1.10 (m, 4H), 1.00 (m, 2H), 0.85 (s, 6H). MS (ESI) m/e 790.4 (M+H)⁺.

### 1.23 Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[4-(1,3-benzothiazol-2-ylcarbamoyl)quinolin-6-yl]pyridine-2-carboxylic acid

### 1.23.1 ethyl 6-(4,4,5,5-tetramethyl-1,3,2-dioxaborolan-2-yl)quinoline-4-carboxylate

To a solution of ethyl 6-bromoquinoline-4-carboxylate (140 mg) in N,N-dimethylformamide (2 mL) was added [1,1′-bis(diphenylphosphino)ferrocene]dichloropalladium(II) dichloromethane (20 mg), potassium acetate (147 mg) and bis(pinacolato)diboron (190 mg). The mixture was stirred at 60° C. overnight. The mixture was cooled to room temperature and used in the next reaction directly. MS (ESI) m/e 328.1 (M+H)⁺.

### 1.23.2 di-tert-butyl {2-[(3,5-dimethyl-7-{[5-methyl-4-(4,4,5,5-tetramethyl-1,3,2-dioxaborolan-2-yl)-1H-pyrazol-1-yl]methyl}tricyclo[3.3.1.1^{3,7}]decan-1-yl)oxy]ethyl}-2-imidodicarbonate

To a solution of Example 1.20.3 (13 g) in dioxane (100 mL) was added dicyclohexyl(2′,6′-dimethoxy-[1,1′-biphenyl]-2-yl)phosphine (S-Phos) (1.0 g) and bis(benzonitrile)palladium(II) chloride (0.23 g) and the reaction was purged with several house vacuum/N₂ refills. 4,4,5,5-Tetramethyl-1,3,2-dioxaborolane (8.8 mL) and triethylamine (8.4 mL) was added followed by a couple more house vacuum/nitrogen refills and then the reaction was heated to 85° C. under nitrogen for 90 minutes. The reaction was cooled, filtered through diatomaceous earth and rinsed with methyl tert-butyl ether. The solution was then concentrated and chromatographed on silica gel using 25% ethyl acetate in heptanes to give the title compound.

### 1.23.3 tert-butyl 3-{1-[(3-{2-[bis(tert-butoxycarbonyl)amino]ethoxy}-5,7-dimethyltricyclo[3.3.1.1^{3,7}]decan-1-yl)methyl]-5-methyl-1H-pyrazol-4-yl}-6-chloropyridine-2-carboxylate

To a solution of Example 1.23.2 (12.3 g) and tert-butyl 3-bromo-6-chloropicolinate (5.9 g) in dioxane (50 mL) was added (1S,3R,5R,7S)-1,3,5,7-tetramethyl-8-phenyl-2,4,6-trioxa-8-phosphaadamantane(CyTop) (0.52 g) and bis(dibenzylidencacetone)palladium(0) (0.66 g). After several house vacuum/nitrogen refills, potassium phosphate (4.06 g) and water (25 mL) were added and the reaction was heated at 80° C. under nitrogen for 30 minutes. The reaction was cooled and then water and ethyl acetate were added. The organic layer was separated and washed with brine. The combined aqueous layers were extracted with ethyl acetate, and dried over sodium sulfate. The solution was filtered, concentrated and chromatographed on silica gel using 33% ethyl acetate in heptanes to give the title compound.

### 1.23.4 ethyl 6-[5-{1-[(3-(2-[bis(tert-butoxycarbonyl)amino]ethoxy)-5,7-dimethyltricyclo[3.3.1.1^{3,7}]dec-1-yl)methyl]-5-methyl-1H-pyrazol-4-yl}-6-(tert-butoxycarbonyl)pyridin-2-yl]quinoline-4-carboxylate

To a solution of Example 1.23.1 (164 mg) in 1,4-dioxane (10 mL) and water (5 mL) was added Example 1.23.3 (365 mg), bis(triphenylphosphine)palladium(II) dichloride (35 mg), and CsF (228 mg). The mixture was stirred at 120° C. for 30 minutes under microwave conditions (Biotage Initiator). The mixture was diluted with ethyl acetate (200 mL) and washed with water and brine and dried over anhydrous sodium sulfate. Filtration and evaporation of the solvent gave a residue that purified by silica gel chromatography, eluting with 20% ethyl acetate in heptane, to give the title compound. MS (ESI) m/e 894.3(M+H)⁺.

### 1.23.5 6-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)quinoline-4-carboxylic acid

To a solution of Example 1.23.4 (3.1 g) in tetrahydrofuran (20 mL), methanol (10 mL) and water (10 mL) was added LiOH H₂O (240 mg). The mixture was stirred at room temperature overnight. The mixture was acidified with aqueous 2N HCl, diluted with ethyl acetate (400 mL), washed with water and brine, and dried over anhydrous sodium sulfate. Filtration and evaporation of the solvent gave the title compound, which was used without further purification. MS (ESI) m/e 766.3(M+H)⁺.

### 1.23.6 3-(1-{([3-(2-aminoethoxy)-5,7-dimethyltricyclo[3.3.1.1^{3,7}]dec-1-yl]methyl}-5-methyl-1H-pyrazol-4-yl)-6-[4-(1,3-benzothiazol-2-ylcarbamoyl)quinolin-6-yl]pyridine-2-carboxylic acid

To a solution of Example 1.23.5 (4.2 g) in dichloromethane (30 mL) was added benzo[d]thiazol-2-amine (728 mg), 1-ethyl-3-[3-(dimethylamino)propyl]-carbodiimide hydrochloride (1.40 g) and 4-(dimethylamino)pyridine (890 mg). The mixture was stirred at room temperature overnight. The reaction mixture was diluted with ethyl acetate (500 mL), washed with water and brine, dried over anhydrous sodium sulfate, filtered and concentrated under reduced pressure. The residue was dissolved in dichloromethane and trifluoroacetic acid (10 mL, 1:1) and stirred overnight. The solvents were removed under reduced pressure. The residue was diluted with N,N-dimethylformamide (2 mL), filtered and purified by reverse-phase HPLC on a Gilson system (C18 column), eluting with 20-80% acetonitrile in water containing 0.10/trifluoroacetic acid, to give the title compound. ¹H NMR (400 MHz, dimethyl sulfoxide-d) δ ppm 9.12 (dd, 1H), 8.92 (s, 1H), 8.61 (dt, 1H), 8.35-8.16 (m, 2H), 8.07 (d, 1H), 7.97-7.87 (m, 2H), 7.81 (d, 1H), 7.66 (s, 3H), 7.53-7.44 (m, 2H), 7.38 (t, 1H), 3.88 (s, 2H), 3.49 (t, 2H), 2.89 (q, 2H), 2.22 (s, 4H), 1.43 (s, 2H), 1.29 (q, 4H), 1.15 (s, 4H), 1.09-0.96 (m, 2H), 0.86 (s, 7H). MS (ESI) m/e 742.2 (M+H)⁺.

### 1.24 Synthesis of 6-[5-amino-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.24.1 5-tert-butoxycarbonylamino-2-(2,2,2-trifluoro-acetyl)-1,2,3,4-tetrahydro-isoquinoline-8-carboxylic acid methyl ester

Example 1.13.4 (5000 mg), tert-butyl carbamate (1920 mg), and cesium carbonate (6674 mg) were added to 1,4-dioxane (80 mL). The solution was degassed and flushed with nitrogen three times. Diacetoxypalladium (307 mg) and (9,9-dimethyl-9H-xanthene-4,5-diyl)bis(diphenylphosphine) (1580 mg) were added, and the solution was degassed and flushed with nitrogen once. The solution was heated to 80° C. for 16 hours. The solution was cooled, and 1 M aqueous HCl (150 mL) was added. The solution was extracted with 50% ethyl acetate in heptanes. The organic portion was washed with brine and dried on anhydrous sodium sulfate. The solution was filtered, concentrated and purified by flash column chromatography on silica gel, eluting with 30% ethyl acetate in heptanes. The solvent was removed under reduced pressure to yield the title compound. MS (ESI) m/e 420 (M+NH₄)⁺, 401 (M−H)⁻.

### 1.24.2 5-tert-butoxycarbonylamino-1,2,3,4-tetrahydro-isoquinoline-8-carboxylic acid methyl ester

The title compound was prepared by substituting Example 1.24.1 for Example 1.13.5 in Example 1.13.6. MS (ESI) m/e 307 (M+H)⁺, 305 (M−H)⁻.

### 1.24.3 2-(5-bromo-6-tert-butoxycarbonyl-pyridin-2-yl)-5-tert-butoxycarbonylamino-1,2,3,4-tetrahydro-isoquinoline-8-carboxylic acid methyl ester

The title compound was prepared by substituting Example 1.24.2 for Example 1.13.6 in Example 1.13.7. MS (ESI) m/e 562, 560 (M+H)⁺, 560, 558 (M−H)⁻.

### 1.24.4 5-tert-butoxycarbonylamino-2-[6-tert-butoxycarbonyl-5-(4,4,5,5-tetramethyl-[1,3,2]dioxaborolan-2-yl)-pyridin-2-yl]-1,2,3,4-tetrahydro-isoquinoline-8-carboxylic acid methyl ester

The title compound was prepared by substituting Example 1.24.3 for Example 1.13.7 in Example 1.13.8. MS (ESI) m/e 610 (M+H)⁺, 608 (M−H)⁻.

### 1.24.5 methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-((tert-butoxycarbonyl)amino)-1,2,3,4-tetrahydroisoquinoline-8-carboxylate

The title compound was prepared by substituting Example 1.24.4 for Example 1.13.8 and Example 1.1.9 for Example 1.4.2 in Example 1.13.9. MS (ESI) m/e 913 (M+H)⁺, 911 (M−H)⁻.

### 1.24.6 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-((tert-butoxycarbonyl)amino)-1,2,3,4-tetrahydroisoquinoline-8-carboxylic acid

The title compound was prepared by substituting Example 1.24.5 for Example 1.13.9 in Example 1.13.10. MS (ESI) m/e 899 (M+H)⁺, 897 (M−H)⁻.

### 1.24.7 tert-butyl 6-(8-(benzo[d]thiazol-2-ylcarbamoyl)-5-((tert-butoxycarbonyl)amino)-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.24.6 for Example 1.13.10 in Example 1.13.11. MS (ESI) m/e 1031 (M+H)⁺, 1029 (M−H)⁻.

### 1.24.8 6-[5-amino-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared by substituting Example 1.24.7 for Example 1.13.11 in Example 1.13.12. ¹H NMR (400 MHz, dimethyl sulfoxide-dₕ) δ ppm 11.42 (s, 1H), 7.98 (d, 1H), 7.75 (d, 1H), 7.55 (d, 1H), 7.44 (t, 2H), 7.31 (t, 1H), 7.27 (s, 1H), 6.92 (d, 1H), 6.58 (d, 1H), 5.74 (s, 2H), 4.99 (s, 2H), 3.93 (t, 2H), 3.82 (s, 2H), 3.57 (s, 3H), 3.54 (m, 2H), 3.09 (q, 2H), 2.98 (bs, 2H), 2.11 (s, 3H), 1.35-1.04 (m, 12H), 0.87 (s, 6H). MS (ESI) m/e 775 (M+H)⁺.

### 1.25 Synthesis of 6-[8-(1,3-benzothiazol-2-ylcarbamoyl)-6-[3-(methylamino)prop-1-yn-1-yl]-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid

### 1.25.1 methyl 6-(3-((tert-butoxycarbonyl)(methyl)amino)prop-1-yn-1-yl)-2-(2,2,2-trifluoroacetyl)-1,2,3,4-tetrahydroisoquinoline-8-carboxylate

A solution of Example 1.21.5 (1.97 g), tert-butyl methyl(prop-2-yn-1-yl)carbamate (1 g), bis(triphenylphosphine)palladium(II) dichloride (0.19 g), CuI (0.041 g), and triethylamine (2.25 mL) in 20 mL dioxane was stirred at 50° C. overnight. The mixture was then concentrated and chromatographed on silica gel using 10-50% ethyl acetate in heptanes to give the title compound.

### 1.25.2 methyl 2-(5-bromo-6-(tert-butoxycarbonyl)pyridin-2-yl)-6-(3-((tert-butoxycarbonyl)(methyl)amino)prop-1-yn-1-yl)-1,2,3,4-tetrahydroisoquinoline-8-carboxylate

The title compound was prepared by substituting Example 1.25.1 for Example 1.21.6 in Example 1.21.7. MS (ESI) m/e 616 (M+H)⁺.

### 1.25.3 methyl 6-(3-((tert-butoxycarbonyl)(methyl)amino)prop-1-yn-1-yl)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

The title compound was prepared by substituting Example 1.25.2 for Example 1.1.9 in Example 1.1.10. MS (ESI) m/e 662.3 (M+H)⁺.

### 1.25.4 methyl 6-(3-((tert-butoxycarbonyl)(methyl)amino)prop-1-yn-1-yl)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared by substituting Example 1.25.3 for Example 1.5.11 and Example 1.17.1 for Example 1.5.10 in Example 1.5.12.

### 1.25.5 6-(3-((tert-butoxycarbonyl)(methyl)amino)prop-1-yn-1-yl)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared by substituting Example 1.25.4 for Example 1.4.7 in Example 1.4.8.

### 1.25.6 tert-butyl 6-(8-(benzo[d]thiazol-2-ylcarbamoyl)-6-(3-((tert-butoxycarbonyl)(methyl)amino)prop-1-yn-1-yl)-3,4-dihydroisoquinolin-2(1H)-yl)-3-(1-((3-(2-methoxyethoxy)-5,7-dimethyladamantan-1-yl)methyl)-5-methyl-1H-pyrazol-4-yl)picolinate

The title compound was prepared by substituting Example 1.25.5 for Example 1.4.8 in Example 1.4.9.

### 1.25.7 6-[8-(1,3-benzothiazol-2-ylcarbamoyl)-6-[3-(methylamino)prop-1-yn-1-yl]-3,4-dihydroisoquinolin-2(1H)-yl]-3-(1-({[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid

The title compound was prepared by substituting Example 1.25.6 for Example 1.21.11 in Example 1.21.12. ¹H NMR (400 MHz, dimethyl sulfoxide-d₆) δ ppm 12.95 (bs, 1H), 8.70 (m, 1H), 8.02 (d, 1H), 7.77 (d, 1H), 7.74 (m, 1H), 7.47 (m, 2H), 7.34 (m, 2H), 7.24 (s, 1H), 6.95 (m, 1H), 6.78 (m, 1H), 4.92 (s, 2H), 4.28 (t, 2H), 3.95 (t, 2H), 3.40 (s, 3H), 3.30 (m, 2H), 3.20 (s, 3H), 3.00 (m, 2H), 2.57 (t, 2H), 2.07 (s, 3H), 1.85 (m, 2H), 1.29 (d, 2H), 1.10-1.24 (m, 10H), 0.85 (s, 6H).

### 1.26 Synthesis of 6-[4-(1,3-benzothiazol-2-ylcarbamoyl)isoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.26.1 methyl 2-(3-bromophenyl)-2-cyanoacetate

To a solution of 2-(3-bromophenyl)acetonitrile (5 g) in tetrahydrofuran (50 mL) was added sodium hydride (3.00 g) portion wise at 23° C. The mixture was heated to 50° C. for 20 minutes. Dimethyl carbonate (8.60 mL) was added dropwise. The mixture was heated at reflux for 2 hours. The mixture was poured into cold and slightly acidic water. The aqueous layer was extracted with ethyl acetate (2×200 mL). The combined organic layers were washed with brine, dried over anhydrous sodium sulfate, filtered through a Buchner funnel and concentrated to give a residue, which was purified by silica gel column chromatography, eluting with 0%-25% dichloromethane/petroleum ether to afford the title compound. MS (LC-MS) m/e 256.0 (M+H)⁺

### 1.26.2 methyl 3-amino-2-(3-bromophenyl)propanoate

Sodium borohydride (14.89 g, 394 mmol) was added portionwise to a solution of Example 1.26.1 (10 g) and cobalt(II) chloride hexahydrate (18.73 g) in methanol (200 mL) at −20° C. The mixture was stirred for 1 hour and the pH was adjusted to 3 with 2N aqueous HCl. The mixture was concentrated. The residue was basified with 2 M aqueous sodium hydroxide and extracted with ethyl acetate. The combined organic layers were dried over anhydrous sodium sulfate, filtered and concentrated to provide the title compound. MS (LC-MS) m/e 260.0 (M+H)⁺.

### 1.26.3 methyl 2-(3-bromophenyl)-3-formamidopropanoate

A solution of Example 1.26.2 (3.6 g) in ethyl formate (54 mL) was heated at 80° C. for 5 hours. The solvent was removed, and the residue was purified by silica gel column chromatography eluting with petroleum/ethyl acetate (2:1-1:2) to give the title compound. MS (LC-MS) m/e 288.0 (M+H)⁺.

### 1.26.4 methyl 8-bromo-2,3-dioxo-3,5,6,10b-tetrahydro-2H-oxazolo[2,3-a]isoquinoline-6-carboxylate

Oxalyl chloride (1.901 mL) was slowly added to a solution of Example 1.26.3 (5.65 g) in dichloromethane (190 mL). The resulting mixture was stirred at 20° C. for 2 hours. The mixture was cooled to −20° C., and iron(III) chloride (3.84 g) was added. The resulting mixture was stirred at 20° C. for 3 hours. Aqueous hydrochloric acid (2M, 45 mL) was added in one portion, and the resulting biphasic mixture was vigorously stirred for 0.5 hours at room temperature. The biphasic mixture was poured into a separatory funnel, and the phases were separated. The organic layer was washed with brine, dried with sodium sulfate, and filtered. The solvent was evaporated under reduced pressure to provide the title compound. The crude product was directly used in subsequent step without purification. MS (LC-MS) m/e 342.0 (M+H)⁺.

### 1.26.5 methyl 6-bromo-3,4-dihydroisoquinoline-4-carboxylate

Example 1.26.4 (13.0 g) in methanol (345 mL) and sulfuric acid (23 mL) was heated at 80° C. for 16 hours. The mixture was concentrated, and the residue was diluted with water, basified with saturated aqueous sodium bicarbonate solution and extracted with ethyl acetate. The combined organic layers were washed with brine, dried over anhydrous sodium sulfate, filtered and concentrated. The residue was purified by silica gel column chromatography, eluting with petroleum ether/ethyl acetate (2:1-1:2) to give the title compound. MS (LC-MS) m/e 268.0 (M+H)⁺.

### 1.26.6 methyl 6-bromoisoquinoline-4-carboxylate

To a solution of Example 1.26.5 (5.25 g) in 1,4-dioxane (200 mL) at 60° C. was added manganese(IV) dioxide (8.5 g). The mixture was heated to 110° C. for 3 hours. The reaction mixture was filtered through a pad of diatomaceous earth and washed with dichloromethane and ethyl acetate. The filtrate was concentrated to dryness. The crude material was adsorbed onto silica gel and purified by silica gel chromatography, eluting with 5-30% ethyl acetate in dichloromethane to give the title compound. MS (LC-MS) m/e 267.9 (M+H)⁺.

### 1.26.7 methyl 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)isoquinoline-4-carboxylate

Example 1.26.6 (229 mg), 4,4,4′,4′,5,5,5′,5′-octamethyl-2,2′-bi(1,3,2-dioxaborolane) (328 mg) and potassium acetate (253 mg) in N,N-dimethylformamide (5 mL) was purged with N₂ for 5 minutes and [1,1′-bis(diphenylphosphino)ferrocene]dichloropalladium(II) dichloromethane (42.2 mg) was added. The mixture was heated at 100° C. overnight and cooled. To the mixture was added Example 1.1.11 (0.369 g), dichlorobis(triphenylphosphine)palladium(II) (0.060 g), cesium fluoride (0.261 g) and water (2 mL). The resulting mixture was heated at 100° C. for 10 hours and filtered. The filtrate was concentrated. The residue was dissolved in dimethyl sulfoxide and purified by reverse-phase HPLC on a Gilson system (C18 column), eluting with 20-80% acetonitrile in water containing 0.1% trifluoroacetic acid, to give the title compound. MS (ESI) m/e 794.5 (M+H)⁺.

### 1.26.8 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)isoquinoline-4-carboxylic acid

Example 1.26.7 (220 mg) in tetrahydrofuran-methanol was treated with 1 M aqueous sodium hydroxide (1.66 mL) for 2 days. The mixture was neutralized with acetic acid and concentrated. The residue was dissolved in dimethyl sulfoxide and purified by reverse-phase HPLC on a Gilson system (C18 column), eluting with 20-80% acetonitrile in water containing 0.1% trifluoroacetic acid, to give the title compound. MS (ESI) m/e 780.5 (M+H)⁺.

### 1.26.9 tert-butyl 6-(4-(benzo[d]thiazol-2-ylcarbamoyl)isoquinolin-6-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a mixture of Example 1.26.8 (122 mg), benzo[d]thiazol-2-amine (47.0 mg), O-(7-azabenzotriazol-1-yl)-N,N,N′,N′-tetramethyluronium hexafluorophosphate (119 mg) in N,N-dimethylformamide (0.5 mL) was added N,N-diisopropylethylamine (273 μL). The mixture was stirred overnight and loaded onto an 80 g silica gel column, eluting with 5-100% heptanes in ethyl acetate to provide the title compound. MS (ESI) m/e 912.5 (M+H)⁺.

### 1.26.10 6-[4-(1,3-benzothiazol-2-ylcarbamoyl)isoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

Example 1.26.9 (100 mg) in dichloromethane (4 mL) was treated with trifluoroacetic acid (2 mL) for 3 hours and the mixture was concentrated. The residue was dissolved in dimethyl sulfoxide (5 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ 13.27 (s, 1H), 9.58 (s, 1H), 9.03 (d, 2H), 8.53 (dd, 1H), 8.42 (d, 1H), 8.25 (t, 3H), 8.06 (d, 1H), 7.97 (d, 1H), 7.81 (d, 1H), 7.56-7.45 (m, 2H), 7.37 (t, 1H), 3.89 (s, 2H), 3.55 (t, 2H), 3.01 (t, 2H), 2.54 (t, 4H), 2.23 (s, 3H), 1.44 (s, 2H), 1.36-1.23 (m, 4H), 1.16 (s, 4H), 0.87 (s, 6H). MS (ESI) m/e 756.1 (M+H)⁺.

### 1.27 Synthesis of 6-[7-(1,3-benzothiazol-2-ylcarbamoyl)-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.27.1 methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1H-indole-7-carboxylate

To a stirred solution of methyl 2-(4,4,5,5-tetramethyl-1,3,2-dioxaborolan-2-yl)-1H-indole-7-carboxylate (370 mg), tris(dibenzylideneacetone)dipalladium(0) (30 mg), 1,2,3,4,5-pentaphenyl-1′-(di-tert-butylphosphino)ferrocene (30 mg) and potassium phosphate (550 mg) in tetrahydrofuran (2 mL) was added Example 1.1.11 (735 mg). The mixture was purged with nitrogen and stirred at 70° C. for 3 hours. The reaction was diluted with ethyl acetate and washed with water and brine. The aqueous layer was back extracted by ethyl acetate. The combined organic layers were dried over sodium sulfate, filtered and concentrated. The residue was purified via silica gel chromatography, eluting with 0-20% ethyl acetate in heptanes, to give the title compound. MS (ESI) m/e 780.4 (M−H)⁺

### 1.27.2 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1H-indole-7-carboxylic acid

The title compound was prepared as described in Example 1.4.8, replacing Example 1.4.7 with Example 1.27.1. MS (ESI) m/e 766.4 (M−H)⁻.

### 1.27.3 tert-butyl 6-(7-(benzo[d]thiazol-2-ylcarbamoyl)-1H-indol-2-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared as described in Example 1.4.9, replacing Example 1.4.8 with Example 1.27.2. MS (ESI) m/e 898.4 (M−H)⁻.

### 1.27.4 6-[7-(1,3-benzothiazol-2-ylcarbamoyl)-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared by substituting Example 1.27.3 for Example 1.1.13 in Example 1.1.14. ¹H NMR (501 MHz, dimethyl sulfoxide-d₆) δ ppm 13.01 (s, 1H), 11.19 (s, 1H), 8.27 (dd, 4H), 8.04 (d, 1H), 7.99 (d, 1H), 7.91 (d, 1H), 7.53-7.45 (m, 3H), 7.36 (t, 1H), 7.27 (t, 1H), 3.91 (s, 2H), 3.57 (t, 3H), 3.03 (t, 3H), 2.58-2.54 (m, 4H), 2.24 (s, 3H), 1.46 (s, 2H), 1.38-1.27 (m, 4H), 1.24-1.01 (m, 6H), 0.89 (s, 6H). MS (ESI) m/e 744.2 (M+H)⁺.

### 1.28 Synthesis of 3-(1-({[3-(2-aminoethoxy)-5,7-dimethyltricyclo[3.3.1.1^{3,7}]dec-1-yl]methyl}-5-methyl-1H-pyrazol-4-yl)-6-[7-(1,3-benzothiazol-2-ylcarbamoyl)-1H-indol-2-yl]pyridine-2-carboxylic acid

### 1.28.1 methyl 2-[5-{1-[(3-{2-[bis(tert-butoxycarbonyl)amino]ethoxy}-5,7-dimethyltricyclo[3.3.1.1^{3,7}]dec-1-yl)methyl]-5-methyl]-1H-pyrazol-4-yl}-6-(tert-butoxycarbonyl)pyridin-2-yl]-1H-indole-7-carboxylate

The title compound was prepared by substituting Example 1.23.3 for Example 1.1.11 in Example 1.27.1. MS (ESI) m/e 866.3 (M−H)⁻.

### 1.28.2 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1H-indole-7-carboxylic acid

The title compound was prepared as described in Example 1.4.8, replacing Example 1.4.7 with Example 1.28.1. MS (ESI) m/e 754.4 (M+H)⁺.

### 1.28.3 tert-butyl 6-(7-(benzo[d]thiazol-2-ylcarbamoyl)-1H-indol-2-yl)-3-(1-((3-(2-((tert-butoxycarbonyl)amino)ethoxy)-5,7-dimethyladamantan-1-yl)methyl)-5-methyl-1H-pyrazol-4-yl)picolinate

The title compound was prepared as described in Example 1.4.9, replacing Example 1.4.8 with Example 1.28.2. MS (ESI) m/e 886.5 (M+H)⁺.

### 1.28.4 3-(1-{[3-(2-aminoethoxy)-5,7-dimethyltricyclo[3.3.1.1^{3,7}]dec-1-yl]methyl}-5-methyl-1H-pyrazol-4-yl)-6-[7-(1,3-benzothiazol-2-ylcarbamoyl)-1H-indol-2-yl]pyridine-2-carboxylic acid

The title compound was prepared by substituting Example 1.28.3 for Example 1.1.13 in Example 1.1.14. ¹H NMR (501 MHz, dimethyl sulfoxide-d₆) δ ppm 13.00 (s, 1H), 11.19 (s, 1H), 8.29 (d, 1H), 8.23 (d, 1H), 8.03 (d, 1H), 7.98 (d, 1H), 7.90 (d, 1H), 7.80 (s, 1H), 7.63 (s, 3H), 7.50 (s, 1H). 7.49-7.44 (m, 2H), 7.39-7.32 (m, 1H), 7.25 (t, 1H), 3.90 (s, 2H), 2.90 (q, 2H), 2.23 (s, 3H), 1.45 (s, 2H), 1.31 (q, 4H), 1.23-1.00 (m, 7H), 0.88 (s, 6H). MS (ESI) m/e 730.2 (M+H)⁺.

### 1.29 Synthesis of 6-[7-(1,3-benzothiazol-2-ylcarbamoyl)-3-methyl-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid 1.29.1 methyl 3-methyl-1H-indole-7-carboxylate

To 7-bromo-3-methyl-1H-indole (1 g), dichloro[1,1′-bis(diphenylphosphino)ferrocene]palladium(II) dichloromethane adduct (0.070 g) in a 50 ml pressure bottle was added methanol (20 mL) and trimethylamine (1.327 mL). The reactor was purged with inert gas, followed by carbon monoxide. The reaction was heated to 100° C. for 20 hours at 60 psi. The solution was filtered and concentrated. The residue was purified by silica gel chromatography, eluting with a gradient of 5-30% ethyl acetate in heptanes, to give the title compound. MS (ESI) m/e 189.9 (M+H)⁺.

### 1.29.2 methyl 2-bromo-3-methyl-1H-indole-7-carboxylate

To a stirred suspension of Example 1.29.1 (70 mg) and 70 mg silica gel in dichloromethane (2 ml) was added 1-bromopyrrolidine-2,5-dione (70 mg). The mixture was protected from light by with aluminum foil and was stirred at room temperature under nitrogen for 30 minutes. The reaction mixture was filtered, washed with dichloromethane and purified via silica gel chromatography, eluting with 10-50% ethyl acetate in heptane, to provide the title compound. MS (ESI) m/e 267.6 (M+H)⁺.

### 1.29.3 methyl 3-methyl-2-(4,4,5,5-tetramethyl-1,3,2-dioxaborolan-2-yl)-1H-indole-7-carboxylate

To a stirred suspension of Example 1.29.2 (398 mg), 4,4′,4′4′,5,5,5′,5′-octamethyl-2,2′-bi(1,3,2-dioxaborolane) (1.2 g) and potassium acetate (450 mg) in 1,4-dioxane (2 ml) was added bis(triphenylphosphine)palladium(II) dichloride (55 mg). The mixture was purged with nitrogen and heated at 115° C. under microwave conditions (Biotage Initiator) for 3 hours. The reaction was diluted with ethyl acetate and washed with water and brine. The aqueous layer was back extracted with ethyl acetate. The combined organic layer was dried over sodium sulfate, filtered and concentrated. The residue was purified via silica gel chromatography, eluting with 5-50% ethyl acetate in heptane, to give the title compound. MS (ESI) m/e 315.9 (M+H)⁺.

### 1.29.4 methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-3-methyl-1H-indole-7-carboxylate

Example 1.29.4 was prepared by substituting Example 1.29.3 for methyl 2-(4,4,5,5-tetramethyl-1,3,2-dioxaborolan-2-yl)-1H-indole-7-carboxylate in Example 1.27.1. MS (ESI) m/e 794.4 (M−H)⁻.

### 1.29.5 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-3-methyl-1H-indole-7-carboxylic acid

Example 1.29.5 was prepared by substituting Example 1.29.4 for Example 1.4.7 in Example 1.4.8. MS (ESI) m/e 780.4 (M−H)⁻.

### 1.29.6 tert-butyl 6-(7-(benzo[d]thiazol-2-ylcarbamoyl)-3-methyl-1H-indol-2-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

Example 1.29.6 was prepared by substituting Example 1.29.5 for Example 1.4.8 in Example 1.4.9. MS (ESI) m/e 912.4 (M−H)⁻.

### 1.29.7 6-[7-(1,3-benzothiazol-2-ylcarbamoyl)-3-methyl-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared by substituting Example 1.29.6 for Example 1.1.13 in Example 1.1.14. ¹H NMR (501 MHz, dimethyl sulfoxide-d₆) δ ppm 12.97 (s, 1H), 11.04 (s, 1H), 8.34-8.23 (m, 3H), 8.06 (d, 1H), 8.02 (dd, 2H), 7.93 (d, 1H), 7.79 (d, 1H), 7.51 (s, 1H), 7.48 (ddd, 1H), 7.38-7.32 (m, 1H), 7.25 (t, 1H), 3.91 (s, 2H), 3.56 (t, 2H), 3.03 (p, 2H), 2.67 (s, 3H), 2.56 (t, 3H), 2.25 (s, 3H), 1.46 (s, 2H), 1.38-1.26 (m, 4H), 1.24-1.13 (m, 4H), 1.06 (q, 2H), 0.89 (s, 6H). MS (ESI) m/e 758.2 (M+H)⁺.

### 1.30 Synthesis of 6-[8-(1,3-benzothiazol-2-ylcarbamoyl)-3,4-dihydroisoquinolin-2(1H)-yl]-3-(1-{[3,5-dimethyl-7-(2-{[1-(methylsulfonyl)piperidin-4-yl]amino}ethoxy)tricyclo[3.3.1.1^{3,7}]dec-1-yl]methyl}-5-methyl-1H-pyrazol-4-yl)pyridine-2-carboxylic acid

### 1.30.1 tert-butyl 6-(8-(benzo[d]thiazol-2-ylcarbamoyl)-3,4-dihydroisoquinolin-2(1H)-yl)-3-(1-(((1r,7r)-3,5-dimethyl-7-(2-((1-(methylsulfonyl)piperidin-4-yl)amino)ethoxy)adamantan-1-yl)methyl)-5-methyl-1H-pyrazol-4-yl)picolinate

A solution of Example 1.18.18 (0.060 g), 1-(methylsulfonyl)piperidin-4-one (0.015 g) and sodium triacetoxyborohydride (0.024 g) was stirred in dichloromethane (0.5 mL) at room temperature. After 30 minutes, the reaction mixture was concentrated. The crude material was dissolved in N,N-dimethylformamide (1.5 mL) and water (0.5 mL) and purified by preparatory reverse-phase HPLC on a Gilson 2020 system using a gradient of 5% to 85% acetonitrile/water. The product-containing fractions were lyophilized to give the title compound as a trifluoroacetic acid salt. MS (ESI) m/e 963.9 (M+H)⁺.

### 1.30.2 6-[8-(1,3-benzothiazol-2-ylcarbamoyl)-3,4-dihydroisoquinolin-2(1H)-yl]-3-(1-{[3,5-dimethyl-7-(2-{[1-(methylsulfonyl)piperidin-4-yl]amino}ethoxy)tricyclo[3.3.1.1^{3,7}]dec-1-yl]methyl}-5-methyl-1H-pyrazol-4-yl)pyridine-2-carboxylic acid

A solution of Example 1.30.1 (0.060 g) was dissolved in dichloromethane (0.5 mL) and treated with trifluoroacetic acid (0.5 mL) overnight. The reaction mixture was concentrated. The residue was dissolved in N,N-dimethylformamide (1.5 mL) and water (0.5 mL) and was purified by preparatory reverse-phase HPLC on a Gilson 2020 system using a gradient of 5% to 85% acetonitrile/water. The product-containing fractions were lyophilized to give the title compound. ¹H NMR (400 MHz, dimethyl sulfoxide-d₆) δ 12.90 (s, 1H), 8.53 (d, 2H), 8.08 (d, 1H), 7.84 (d, 1H), 7.66 (d, 1H), 7.58-7.45 (m, 4H), 7.41 (td, 2H), 7.33 (s, 1H), 7.00 (d, 1H), 5.00 (s, 2H), 3.93 (s, 2H), 3.88 (s, 2H), 3.62 (d, 4H), 3.22 (h, 2H), 3.12, 3.06 (s, 2H), 2.93 (s, 3H), 2.79 (d, 2H), 2.15 (s, 3H), 2.11 (s, 1H), 1.61 (qd, 2H), 1.48 (s, 2H), 1.37 (s, 2H), 1.19 (s, 4H), 1.10 (s, 2H), 0.91 (s, 8H). MS (ESI) m/e 907.2 (M+H)⁺.

### 1.31 Synthesis of 6-[8-(1,3-benzothiazol-2-ylcarbamoyl)-3,4-dihydroisoquinolin-2(1H)-yl]-3-(1-{[3,5-dimethyl-7-(2-{[1-(methylsulfonyl)azetidin-3-yl]amino}ethoxy)tricyclo[3.3.1.1^{3,7}]dec-1-yl]methyl}-5-methyl-1H-pyrazol-4-yl)pyridine-2-carboxylic acid

A solution of Example 1.18.18 (0.050 g), 1-(methylsulfonyl)azetidin-3-one (0.014 g) and sodium triacetoxyborohydride (0.020 g) was stirred in dichloromethane (0.50 mL) at room temperature. After 30 minutes, acetic acid (5.35 μL) was added and stirring was continued at room temperature overnight. Trifluoroacetic acid (0.5 mL) was added to the reaction and was stirring continued overnight. The reaction mixture was concentrated. The residue was dissolved in a mixture of N,N-dimethylformamide (2 mL) and water (0.5 mL) and was purified by preparatory reverse-phase HPLC on a Gilson 2020 system using a gradient of 5% to 70% acetonitrile/water. The product-containing fractions were lyophilized to give the title compound. ¹H NMR (400 MHz, dimethyl sulfoxide-d₆) δ 12.86 (s, 1H), 9.13 (s, 2H), 8.03 (d, 1H), 7.79 (d, 1H), 7.62 (d, 1H), 7.54-7.41 (m, 3H), 7.36 (td, 2H), 7.29 (s, 1H), 6.96 (d, 1H), 4.96 (s, 2H), 4.09 (s, 2H), 4.08 (s, 1H), 3.98 (s, 2H), 3.89 (s, 2H), 3.84 (s, 2H), 3.56 (s, 2H), 3.05 (s, 3H), 3.03 (s, 2H), 3.02 (s, 1H), 2.11 (s, 2H), 1.44 (s, 2H), 1.31 (q, 4H), 1.14 (s, 4H), 1.06 (s, 2H), 0.87 (s, 6H). MS (ESI) m/e 879.7 (M+H)⁺.

### 1.32 Synthesis of 3-{1-[(3-{2-[(3-amino-3-oxopropyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid

### 1.32.1 tert-butyl 3-(1-((3-(2-((3-amino-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

A mixture of Example 1.18.18 (245 mg) and acrylamide (217 mg) in N,N-dimethylformamide (5 mL) was heated at 50° C. for 3 days and was purified by reverse phase HPLC, eluted with 30%-80% acetonitrile in 0.10/1% trifluoroacetic acid in water solution, to provide the title compound. ¹H NMR (400 MHz, dimethyl sulfoxide-d₆) δ 12.83 (s, 1H), 8.30 (s, 2H), 8.00 (dd, 1H), 7.76 (d, 1H), 7.57 (d, 2H), 7.44 (ddd, 3H), 7.39-7.29 (m, 2H), 7.21 (s, 1H), 7.13 (s, 1H), 6.91 (d, 1H), 4.95 (s, 2H), 3.81 (d, 4H), 3.53 (t, 2H), 3.05 (dq, 6H), 2.06 (s, 3H), 1.43 (s, 2H), 1.27 (q, 4H), 1.13 (d, 15H), 0.82 (s, 6H). MS (ESI) m/e 873.8 (M+H)⁺.

### 1.32.2 3-{1-[(3-{2-[(3-amino-3-oxopropyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid

The title compound was prepared using the procedure in Example 1.26.10, replacing Example 1.26.9 with Example 1.32.1. ¹H NMR (400 MHz, dimethyl sulfoxide-d₆) δ 8.29 (s, 2H), 8.00 (dd, 1H), 7.76 (d, 1H), 7.63-7.52 (m, 2H), 7.49-7.38 (m, 3H), 7.37-7.29 (m, 2H), 7.25 (s, 1H), 7.11 (s, 1H), 6.92 (d, 1H), 4.92 (s, 2H), 3.53 (t, 2H), 3.04 (ddt, 6H), 2.07 (s, 3H), 1.39 (s, 2H), 1.26 (q, 4H), 1.16-0.93 (m, 6H), 0.83 (s, 6H). MS (ESI) m/e 817.2 (M+H)⁺.

### 1.33 Synthesis of 6-[3-(1,3-benzothiazol-2-ylcarbamoyl)-1H-indazol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.33.1 5-(4,4,5,5-tetramethyl-[1,3,2]dioxaborolan-2-yl)-1-(2-trimethylsilanyl-ethoxymethyl)-1H-indazole-3-carboxylic acid ethyl ester

Ethyl 5-(4,4,5,5-tetramethyl-1,3,2-dioxaborolan-2-yl)-1H-indazole-3-carboxylate (1000 mg) was dissolved in N,N-dimethylformamide (30 mL). Sodium hydride (60% in mineral oil, 83 mg) was added, and the solution and was stirred at room temperature for 20 minutes. (2-(Chloromethoxy)ethyl)trimethylsilane (580 mg) was added, and the solution was stirred at room temperature for 90 minutes. The reaction was quenched with saturated aqueous ammonium chloride (10 mL) and diluted with water (90 mL). The solution was extracted with 70% ethyl acetate in heptanes (50 mL) twice. The combined organic portions were washed with water (25 mL) and then brine (25 mL). The solution was dried on anhydrous sodium sulfate, filtered and concentrated under reduced pressure. The residue was purified by flash column chromatography on silica gel, eluting with 10-30% ethyl acetate in heptanes. The solvent was removed under reduced pressure to yield the title compound. MS (ESI) m/e 447 (M+H)⁺.

### 1.33.2 ethyl 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indazole-3-carboxylate

Example 1.33.1 (335 mg) and Example 1.1.11 (483 mg) were dissolved in 1,4-dioxane (3 mL). 2 M aqueous sodium carbonate (1.13 mL) was added, and the solution was degassed and flushed with nitrogen three times. Dichloro[1,1′-bis(diphenylphosphino)ferrocene]palladium(II) (61 mg) was added, and the solution was degassed and flushed with nitrogen once. The solution was heated at 75° C. for 16 hours. The solution was cooled, and 0.1 M aqueous HCl (25 mL) was added. The solution was extracted with ethyl acetate (50 mL) twice. The combined organic portions were washed with brine (25 mL) and dried on anhydrous sodium sulfate. The solution was filtered, concentrated under reduced pressure and purified by flash column chromatography on silica gel, eluting with 50% ethyl acetate in heptanes. The solvent was removed under reduced pressure to yield the title compound. MS (ESI) m/e 927 (M+NH₄—H₂O).

### 1.33.3 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indazole-3-carboxylic acid

The title compound was prepared by substituting Example 1.33.2 for Example 1.13.9 in Example 1.13.10. MS (ESI) m/e 899 (M+H)⁺, 897 (M−H)⁻.

### 1.33.4 tert-butyl 6-(3-(benzo[d]thiazol-2-ylcarbamoyl)-1-((2-(trimethylsilyl)ethoxy)methyl)-1H-indazol-5-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.33.3 for Example 1.13.10 in Example 1.13.11. MS (ESI) m/e 1030 (M+NH₄—H₂O)⁺, 1029 (M−H)⁻.

### 1.33.5 6-[3-(1,3-benzothiazol-2-ylcarbamoyl)-1H-indazol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

Example 1.33.4 (83 mg) was dissolved in dichloromethane (0.5 mL). Trifluoroacetic acid (740 mg) was added, and the solution was stirred at room temperature for 16 hours. The solvents were removed under reduced pressure. The residue was dissolved in 1,4-dioxane (1 mL), and 1 M aqueous sodium hydroxide (0.5 mL) was added. The solution was stirred at room temperature for 60 minutes. The reaction was quenched with trifluoroacetic acid (0.1 mL) and purified by reverse-phase HPLC using 10-85% acetonitrile in water (w/0.1% trifluoroacetic acid) over 30 minutes on a Grace Reveleris equipped with a Luna column: C18(2), 100 A, 150×30 mm. Product fractions were combined, frozen, and lyophilized to yield the title compound as the bis trifluoroacetic acid salt. ¹H NMR (400 MHz, dimethyl sulfoxide-d₆) δ ppm 14.23 (s, 1H), 12.58 (bs, 1H), 8.97 (s, 1H), 8.34-8.29 (m, 3H), 8.22 (d, 1H), 8.04 (d, 1H), 7.91 (d, 1H), 7.87-7.81 (m, 2H), 7.51-7.45 (m, 2H), 7.36 (t, 1H), 3.92 (s, 3H), 3.58 (m, 2H), 3.04 (m, 2H), 2.58-2.56 (m, 2H), 2.26 (s, 3H), 1.47 (s, 2H), 1.34 (q, 4H), 1.22-1.14 (m, 4H), 1.07 (q, 2H), 0.89 (m, 6H). MS (ESI) m/e 745 (M+H)⁺, 743 (M−H)⁻.

### 1.34 Synthesis of 6-[3-(1,3-benzothiazol-2-ylcarbamoyl)-1H-indol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.34.1 5-(4,4,5,5-tetramethyl-[1,3,2]dioxaborolan-2-yl)-1-(2-trimethylsilanyl-ethoxymethyl)-1H-indole-3-carboxylic acid methyl ester

The title compound was prepared by substituting methyl 5-(4,4,5,5-tetramethyl-1,3,2-dioxaborolan-2-yl)-1H-indole-3-carboxylate for ethyl 5-(4,4,5,5-tetramethyl-1,3,2-dioxaborolan-2-yl)-1H-indazole-3-carboxylate in Example 1.33.1. MS (ESI) m/e 432 (M+H)⁺.

### 1.34.2 methyl 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indole-3-carboxylate

The title compound was prepared by substituting Example 1.34.1 for Example 1.33.1 in Example 1.33.2. MS (ESI) m/e 912 (M+H)⁺.

### 1.34.3 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indole-3-carboxylic acid

The title compound was prepared by substituting Example 1.34.2 for Example 1.13.9 in Example 1.13.10. MS (ESI) m/e 898 (M+H)⁺, 896 (M−H)⁻.

### 1.34.4 tert-butyl 6-(3-(benzo[d]thiazol-2-ylcarbamoyl)-1-((2-(trimethylsilyl)ethoxy)methyl)-1H-indol-5-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.34.3 for Example 1.13.10 in Example 1.13.11. MS (ESI) m/e 1030 (M+H)⁺, 1028 (M−H)⁻.

### 1.34.5 6-[3-(1,3-benzothiazol-2-ylcarbamoyl)-1H-indol-5-yl]-3-[1-(3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared by substituting Example 1.34.4 for Example 1.33.4 in Example 1.33.5. ¹H NMR (400 MHz, dimethyl sulfoxide-d₆) δ ppm 12.47 (bs, 1H), 12.18 (s, 1H), 9.01 (s, 1H), 8.70 (d, 1H), 8.28 (bs, 3H), 8.12 (d, 1H), 8.05 (dd, 1H), 7.99 (d, 1H), 7.86 (d, 1H), 7.76 (d, 1H), 7.64 (d, 1H), 7.50 (s, 1H), 7.46 (td, 1H), 7.32 (t, 1H), 3.92 (s, 3H), 3.58 (m, 2H), 3.04 (m, 2H), 2.57 (m, 2H), 2.26 (s, 3H), 1.47 (s, 2H), 1.34 (q, 4H), 1.24-1.14 (m, 4H), 1.08 (m, 2H), 0.90 (s, 6H). MS (ESI) m/e 744 (M+H)⁺, 742 (M−H)⁻.

### 1.35 Synthesis of 6-[3-(1,3-benzothiazol-2-ylcarbamoyl)-1H-pyrrolo[2,3-b]pyridin-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

### 1.35.1 5-bromo-1-(2-trimethylsilanyl-ethoxymethyl)-1H-pyrrolo[2,3-b]pyridine-3-carboxylic acid methyl ester

The title compound was prepared by substituting methyl 5-bromo-1H-pyrrolo[2,3-b]pyridine-3-carboxylate for ethyl 5-(4,4,5,5-tetramethyl-1,3,2-dioxaborolan-2-yl)-1H-indazole-3-carboxylate in Example 1.33.1. MS (ESI) m/e 385, 387 (M+H)⁺.

### 1.35.2 5-(4,4,5,5-tetramethyl-[1,3,2]dioxaborolan-2-yl)-1-(2-trimethylsilanyl-ethoxymethyl)-1H-pyrrolo[2,3-b]pyridine-3-carboxylic acid methyl ester

The title compound was prepared by substituting Example 1.35.1 for Example 1.13.7 in Example 1.13.8. MS (ESI) m/e 433(M+H)⁺.

### 1.35.3 methyl 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-pyrrolo[2,3-b]pyridine-3-carboxylate

The title compound was prepared by substituting Example 1.35.2 for Example 1.33.1 in Example 1.33.2. MS (ESI) m/e 913 (M+H)⁺.

### 1.35.4 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-pyrrolo[2,3-b]pyridine-3-carboxylic acid

The title compound was prepared by substituting Example 1.35.3 for Example 1.13.9 in Example 1.13.10. MS (ESI) m/e 899 (M+H)⁺, 897 (M−H)⁻.

### 1.35.5 tert-butyl 6-(3-(benzo[d]thiazol-2-ylcarbamoyl)-1-((2-(trimethylsilyl)ethoxy)methyl)-1H-pyrrolo[2,3-b]pyridin-5-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.35.4 for Example 1.13.10 in Example 1.13.11. MS (ESI) m/e 1031 (M+H)⁺, 1029 (M−H)⁻.

### 1.35.6 6-[3-(1,3-benzothiazol-2-ylcarbamoyl)-1H-pyrrolo[2,3-b]pyridin-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared by substituting Example 1.35.5 for Example 1.33.4 in Example 1.33.5. ¹H NMR (400 MHz, dimethyl sulfoxide-d₆) δ ppm 12.74 (d, 1H), 12.62 (bs, 1H), 9.26 (d, 1H), 9.13 (d, 1H), 8.83 (d, 1H), 8.28 (bs, 2H), 8.25 (d, 1H), 7.99 (d, 1H), 7.91 (d, 1H), 7.78 (d, 1H), 7.51 (s, 1H), 7.47 (t, 1H), 7.33 (t, 1H), 3.92 (s, 3H), 3.58 (t, 2H), 3.04 (m, 2H), 2.57 (t, 2H), 2.26 (s, 3H), 1.47 (s, 2H), 1.34 (q, 4H), 1.20 (t, 4H), 1.08 (q, 2H), 0.90 (s, 6H). MS (ESI) m/e 745 (M+H)⁺, 743 (M−H)⁻.

### 1.36 Synthesis of 6-(8-(benzo[d]thiazol-2-ylcarbamoyl)-3,4-dihydroisoquinolin-2(1H)-yl)-3-(1-((3-(2-((2-(N,N-dimethylsulfamoyl)ethyl)amino)ethoxy)-5,7-dimethyladamantan-1-yl)methyl)-5-methyl-1H-pyrazol-4-yl)picolinic acid

To a solution of Example 1.18.18 (69.8 mg) in N,N-dimethylformamide (6 mL) was added N,N-dimethylethenesulfonamide (118 mg), N,N-diisopropylethylamine (0.2 mL) and H₂O (0.2 mL). The mixture was stirred at room temperature 4 days. The reaction mixture was diluted with ethyl acetate (200 mL), washed with water and brine, and dried over anhydrous sodium sulfate. After evaporation of the solvent, the residue was dissolved in dichloromethane and trifluoroacetic acid (10 mL, 1:1), and the resulting solution was stirred overnight. The solvents were removed under reduced pressure. The residue was diluted with N,N-dimethylformamide (2 mL), filtered and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 12.82 (s, 1H), 8.53 (s, 2H), 8.00 (dd, 1H), 7.76 (d, 1H), 7.59 (dd, 1H), 7.53-7.37 (m, 4H), 7.37-7.28 (m, 2H), 7.26 (s, 1H), 6.92 (d, 1H), 4.92 (s, 2H), 3.80 (s, 2H), 3.54 (t, 2H), 3.44-3.34 (m, 2H), 3.30 (s, 2H), 3.11 (s, 2H), 2.98 (t, 2H), 2.77 (s, 6H), 2.07 (s, 3H), 1.39 (s, 2H), 1.27 (q, 4H), 1.11 (s, 4H), 1.06-0.93 (m, 2H), 0.83 (s, 7H). MS (ESI) m/e 881.2 (M+H)⁺.

### 1.37 Synthesis of 6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.1^{3,7}]dec-1-yl)methyl]-5-methyl-1H-pyrazol-4-yl}pyridine-2-carboxylic acid

### 1.37.1 2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethanol

To a solution of Example 1.1.6 (8.9 g) and [1,1′-bis(diphenylphosphino)ferrocene]dichloropalladium(II) dichloromethane (818 mg) in acetonitrile (120 mL) was added triethylamine (10 mL) and pinacolborane (12.8 mL). The mixture was stirred at reflux overnight. The mixture was cooled to room temperature and used in the next reaction directly. MS (ESI) m/e 467.3 (M+Na)⁺.

### 1.37.2 tert-butyl 6-chloro-3-(1-((3-(2-hydroxyethoxy)-5,7-dimethyladamantan-1-yl)methyl)-5-methyl-1H-pyrazol-4-yl)picolinate

To a solution of tert-butyl 3-bromo-6-chloropicolinate (6.52 g) in tetrahydrofuran (100 mL) and water (20 mL) was added Example 1.37.1 (9.90 g), (1S,31R5R,7S)-1,3,5,7-tetramethyl-8-tetradccyl-2,4,6-trioxa-8-phosphaadamantane (0.732 g), tris(dibenzylideneacetone)dipalladium(0) (1.02 g), and potassium phosphate (23.64 g), and the mixture was stirred at reflux overnight. The solvents were removed under vacuum. The residue was dissolved in ethyl acetate (500 mL), washed with water and brine, and dried over anhydrous sodium sulfate. Filtration and evaporation of the solvent gave a residue that purified by silica gel chromatography, eluting with 20% ethyl acetate in heptane, to give the title compound. MS (ESI) m/e 530.3 (M+H)⁺.

### 1.37.3 tert-butyl 3-{1-[(3-{2-[bis(tert-butoxycarbonyl)amino]ethoxy}-5,7-dimethyltricyclo[3.3.1.1^{3,7}]decan-1-yl)methyl]-5-methyl-1H-pyrazol-4-yl}-6-chloropyridine-2-carboxylatetert-butyl 6-chloro-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) stirring solution of Example 1.37.2 (3.88 g) in dichloromethane (30 mL) and triethylamine (6 mL) was added methanesulfonyl chloride (2.52 g). The mixture was stirred at room temperature for 4 hours. The reaction mixture was diluted with ethyl acetate (400 mL), washed with water and brine, and dried over anhydrous sodium sulfate. Filtration and evaporation of the solvent gave the title compound, which was used in the next reaction without further purification. MS (ESI) m/e 608.1 (M+H)⁺.

### 1.37.4 tert-butyl 3-{1-[(3-{2-[bis(tert-butoxycarbonyl)amino]ethoxy}-5,7-dimethyltricyclo[3.3.1.1^{3,7}]dec-1-yl)methyl]-5-methyl-1H-pyrazol-4-yl}-6-chloropyridine-2-carboxylate

To a solution of Example 1.37.3 (151 mg) in N,N-dimethylformamide (3 mL) was added di-t-butyl iminodicarboxylate (54 mg). The mixture was stirred at room temperature overnight. The reaction mixture was diluted with ethyl acetate (200 mL), washed with water and brine, and dried over anhydrous sodium sulfate. Filtration and evaporation of the solvent gave the title compound, which was used in the next step without further purification. MS (ESI) m/e 729.4 (M+H)⁺.

### 1.37.5 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1-naphthoic acid

To a solution of methyl 7-(4,4,5,5-tetramethyl-1,3,2-dioxaborolan-2-yl)-1-naphthoate (257 mg) in 1,4-dioxane (10 mL) and water (5 mL) was added Example 1.37.4 (600 mg), bis(triphenylphosphine)palladium(II) dichloride (57.8 mg), and cesium fluoride (375 mg). The mixture was stirred at 120° C. for 30 minutes under microwave conditions (Biotage Initiator). The mixture was diluted with ethyl acetate (200 mL), washed with water and brine, dried over anhydrous sodium sulfate, filtered and concentrated. Evaporation of the solvent gave a residue that purified by silica gel chromatography, eluting with 20% ethyl acetate in heptane, to give an intermediate di-ester. The residue was dissolved in tetrahydrofuran (10 mL), methanol (5 mL) and water (5 mL) and LiOH H₂O (500 mg) was added. The mixture was stirred at room temperature overnight. The mixture was acidified with aqueous 2N HCl, dissolved in 400 mL of ethyl acetate, washed with water and brine and dried over anhydrous sodium sulfate. Filtration and evaporation of the solvent gave the title compound. MS (APCI) m/e 765.3 (M+H)⁺.

### 1.37.6 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a solution of Example 1.37.5 (500 mg) in dichloromethane (10 mL) was added benzo[d]thiazol-2-amine (98 mg), 1-ethyl-3-[3-(dimethylamino)propyl]-carbodiimide hydrochloride (251 mg) and 4-(dimethylamino)pyridine (160 mg). The mixture was stirred at room temperature overnight. The reaction mixture was diluted with ethyl acetate (400 mL), washed with water and brine, dried over anhydrous sodium sulfate, filtered and concentrated. The residue was dissolved in dichloromethane and trifluoroacetic acid (10 mL, 1:1), and the solution was stirred overnight. The solvents were removed, and the residue was dissolved in N,N-dimethylformamide (12 mL) and purified by reverse-phase HPLC on a Gilson system (C18 column), eluting with 20-80% acetonitrile in water containing 0.1% trifluoroacetic acid, to give the title compound. MS (ESI) m/e 741.2 (M+H)⁺.

### 1.37.7 3-((tert-butyldimethylsilyl)oxy)propanal

To a solution of dimethyl sulfoxide (2.5 mL) in dichloromethane (40 mL) at −78° C. was added oxalyl chloride (1.5 mL). The mixture was stirred 20 minutes at −78° C., and a solution of (3-((tert-butyldimethylsilyl)oxy)propan-1-ol (1.9 g) in dichloromethane (10 mL) was added by syringe. After 1 hour, triethylamine (5 mL) was added. The cooling bath was removed, and the reaction was stirred overnight. The reaction mixture was diluted with ethyl acetate (300 mL), washed with water and brine, and dried over anhydrous sodium sulfate. Filtration and evaporation of solvent gave the title compound. MS (DCI) m/e 206.0(M+NH₄)⁺.

### 1.37.8 6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.1^{3,7}]dec-1-yl)methyl]-5-methyl-1H-pyrazol-4-yl}pyridine-2-carboxylic acid

To a solution of Example 1.37.6 (125 mg) in dichloromethane (10 mL) was added Example 1.37.7 (32 mg). The mixture was stirred at room temperature for 1 hour, and NaBH(OAc)₃ (107 mg) was added to the reaction mixture. The mixture was stirred at room temperature overnight. To the reaction mixture was added 2N aqueous sodium hydroxide (5 mL), and the reaction stirred for 4 hours. The mixture was neutralized with aqueous 2N HCl and extracted with ethyl acetate (100 mL×3). The combined organic layers were washed with aqueous 2% HCl, water and brine and dried over anhydrous sodium sulfate. Filtration and evaporation of the solvent gave a residue that was purified by reverse-phase HPLC on a Gilson system (C18 column), eluting with 20-80% acetonitrile in water containing 0.1% trifluoroacetic acid, to give a solid. The residue was dissolved in tetrahydrofuran (6 mL) and tetrabutyl ammonium fluoride (1 M in tetrahydrofuran, 4 mL) was added. The mixture was stirred at room temperature for 2 hours, and the solvents were removed under vacuum. The residue was dissolved in dimethyl sulfoxide/methanol (1:1, 12 mL) and was purified by reverse-phase HPLC on a Gilson system (C18 column), eluting with 20-80% acetonitrile in water containing 0.1% trifluoroacetic acid, to give the title compound. ¹H NMR (501 MHz, dimethyl sulfoxide-d₆) δ ppm 13.09 (s, 1H), 9.01 (s, 1H), 8.36 (dd, 1H), 8.20 (ddd, 5H), 8.09-8.02 (m, 1H), 8.03-7.95 (m, 1H), 7.92 (d, 1H), 7.80 (d, 1H), 7.69 (dd, 1H), 7.53-7.43 (m, 2H), 7.36 (ddd, 1H), 3.89 (s, 2H), 3.56 (t, 2H), 3.47 (t, 2H), 3.10-2.93 (m, 4H), 2.22 (s, 3H), 1.78-1.68 (m, 2H), 1.44 (s, 2H), 1.30 (q, 4H), 1.20-1.11 (m, 4H), 1.04 (q, 2H), 0.87 (s, 7H). MS (ESI) m/e 799.2 (M+H)⁺.

### 1.38 Synthesis of 6-[8-(1,3-benzothiazol-2-ylcarbamoyl)-3,4-dihydroisoquinolin-2(1H)-yl]-3-(1-{[3-(2-{[3-(dimethylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid

To a solution of Example 1.18.18 (55 mg) in N,N-dimethylformamide (6 mL) was added N,N-dimethylacrylamide (73.4 mg), N,N-diisopropylethylamine (0.2 mL) and water (0.2 mL). The mixture was stirred at room temperature 4 days. The reaction mixture was diluted with ethyl acetate (200 mL), washed with water and brine, and dried over anhydrous sodium sulfate. After filtration and evaporation of the solvent, the residue was dissolved in dichloromethane and trifluoroacetic acid (10 mL, 1:1). After stirring for 16 hours, the mixture was concentrated under reduced pressure. The residue was dissolved in N,N-dimethylformamide (8 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 12.84 (s, 1H), 8.22 (s, 3H), 8.02 (d, 1H), 7.78 (d, 1H), 7.60 (d, 1H), 7.55-7.39 (m, 3H), 7.39-7.30 (m, 2H), 7.27 (s, 1H), 6.94 (d, 1H), 4.94 (s, 2H), 3.87 (t, 2H), 3.81 (s, 2H), 3.55 (t, 2H), 3.20-2.95 (m, 6H), 2.92 (s, 3H), 2.82 (s, 3H), 2.69 (q, 3H), 2.09 (s, 3H), 1.40 (s, 2H), 1.28 (q, 4H), 1.14 (d, 4H), 1.07-0.94 (m, 2H), 0.85 (s, 8H). MS (ESI) m/e 845.3 (M+H)⁺.

### 1.39 Synthesis of 6-[8-(1,3-benzothiazol-2-ylcarbamoyl)-3,4-dihydroisoquinolin-2(1H)-yl]-3-(1-{[3,5-dimethyl-7-(2-{[3-(methylamino)-3-oxopropyl]amino}ethoxy)tricyclo[3.3.1.1^{3,7}]dec-1-yl]methyl}-5-methyl-1H-pyrazol-4-yl)pyridine-2-carboxylic acid

The title compound was prepared as described in Example 1.38, by replacing N,N-dimethylacrylamide with N-methylacrylamide. ¹H NMR (501 MHz, dimethyl sulfoxide-d₆) δ ppm 12.84 (s, 1H), 8.32 (s, 2H), 8.08-7.96 (m, 2H), 7.78 (d, 1H), 7.60 (d, 1H), 7.52-7.40 (m, 3H), 7.39-7.30 (m, 2H), 7.27 (s, 1H), 6.94 (d, 1H), 4.94 (s, 2H), 3.87 (t, 2H), 3.81 (s, 2H), 3.12 (p, 2H), 3.01 (dt, 4H), 2.57 (d, 3H), 2.09 (s, 3H), 1.40 (s, 2H), 1.28 (q, 5H), 1.18-1.07 (m, 4H), 1.02 (q, 2H), 0.85 (s, 7H). MS (ESI) m/e 831.3 (M+H)⁺.

### 1.40 Synthesis of 3-(1-{[3-(2-aminoacetamido)-5,7-dimethyltricyclo[3.3.1.1^{3,7}]decan-1-yl]methyl}-5-methyl-1H-pyrazol-4-yl)-6-{8-[(1,3-benzothiazol-2-yl)carbamoyl]-3,4-dihydroisoquinolin-2(1H)-yl}pyridine-2-carboxylic acid

### 1.40.1 1-((3-bromo-5,7-dimethyladamantan-1-yl)methyl)-5-methyl-1H-pyrazole

To a cooled (−30° C.) solution of Example 1.1.3 (500 mg) in tetrahydrofuran (30 mL) was added n-butyllithium (9.67 mL), and the mixture was stirred at −30° C. for 2 hours. Methyl iodide (1.934 mL) was added dropwise at −30° C. After completion of the addition, the mixture was stirred at −30° C. for additional 2 hours. 1N aqueous HCl in ice water was added slowly, such that the temperature was maintained below 0° C., until the pH reached 6. The mixture was stirred at room temperature for 10 minutes, and was diluted with ice-water (10 mL) and ethyl acetate (20 mL). The layers were separated, and the aqueous was extracted twice with ethyl acetate. The combined organic phases were washed with brine, dried over MgSO₄, filtered and concentrated. The residue was purified by flash silica gel chromatography, eluting with 15/1 to 10/1 petroleum/ethyl acetate, to give the title compound. MS (LC-MS) m/e 337, 339 (M+H)⁺.

### 1.40.2 1-(3,5-dimethyl-7-((5-methyl-1H-pyrazol-1-yl)methyl)adamantan-1-yl)urea

Example 1.40.1 (2.7 g) and urea (4.81 g) were mixed and stirred at 140° C. for 16 hours. The mixture was cooled to room temperature and suspended in methanol (200 mL×2). The insoluble material was removed by filtration. The filtrate was concentrated to give the title compound. MS (LC-MS) m/e 317.3 (M+H)⁺.

### 1.40.3 3,5-dimethyl-7-((5-methyl-1H-pyrazol-1-yl)methyl)adamantan-1-amine

To a solution of Example 1.40.2 (2.53 g) in 20% o ethanol in water (20 mL) was added sodium hydroxide (12.79 g). The mixture was stirred at 120° C. for 16 hours and at 140° C. for another 16 hours. 6N Aqueous HCl was added until the pH reached 6. The mixture was concentrated, and the residue was suspended in methanol (200 mL). The insoluble material was filtered off. The filtrate was concentrated to give the title compound as an HCl salt. MS (LC-MS) m/e 273.9 (M+H)⁺.

### 1.40.4 tert-butyl (2-((3,5-dimethyl-7-((5-methyl-1H-pyrazol-1-yl)methyl)adamantan-1-yl)amino)-2-oxoethyl)carbamate

To a solution of Example 1.40.3 (2.16 g) in N,N-dimethylformamide (100 mL) was added triethylamine (3.30 mL), 2-((tert-butoxycarbonyl)amino)acetic acid (1.799 g) and O-(7-azabenzotriazol-1-yl)-N,N,N′,N′-tetramethyluronium hexafluorophosphate (3.90 g). The mixture was stirred at room temperature for 2 hours. Water (40 mL) was added, and the mixture was extracted with ethyl acetate (70 mL×2). The combined organic phases were washed with brine, dried over sodium sulfate, filtered and concentrated. The residue was purified by silica gel chromatography, eluting with 3/1 to 2/1 petroleum/ethyl acetate, to give the title compound. MS (LC-MS) m/e 430.8 (M+H)⁺.

### 1.40.5 tert-butyl (2-((3-((4-iodo-5-methyl-1H-pyrazol-1-yl)methyl)-5,7-dimethyladamantan-1-yl)amino)-2-oxoethyl)carbamate

To an ambient solution of Example 1.40.4 (1.7 g) in N,N-dimethylformamide (20 mL) was added N-iodosuccinimide (1.066 g) in portions, and the mixture was stirred at room temperature for 16 hours. Ice-water (10 mL) and saturated aqueous Na₂S₂O₃ solution (10 mL) were added. The mixture was extracted with ethyl acetate (30 mL×2). The combined organic phases were washed with brine, dried over sodium sulfate, filtered and concentrated. The residue was purified by silica gel chromatography, eluting with 3/1 to 2/1 petroleum/ethyl acetate, to give the title compound. MS (LC-MS) m/e 556.6 (M+H)-.

### 1.40.6 methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.4.4 (15 g) in dimethyl sulfoxide (100 mL) was added N,N-diisopropylethylamine (12 mL), and the mixture was stirred at 50° C. for 24 hours. The mixture was then diluted with ethyl acetate (500 mL) and washed with water and brine. The organic layer was dried over sodium sulfate, filtered and concentrated under reduced pressure. The residue was purified by silica gel chromatography, eluting with 20% ethyl acetate in hexane, to give the title compound. MS (ESI) m/e 448.4 (M+H)⁺.

### 1.40.7 methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.40.6 (2.25 g) and [1,1′-bis(diphenylphosphino)ferrocene]dichloropalladium(II) (205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered and concentrated under reduced pressure. Purification of the residue by flash chromatography, eluting with 20% ethyl acetate in hexane, provided the title compound.

### 1.40.8 methyl 2-(6-(tert-butoxycarbonyl)-5-(1-((3-(2-((tert-butoxycarbonyl)amino)acetamido)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared using the procedure in Example 1.4.7, replacing Example 1.4.6 and Example 1.4.2 with Example 1.40.7 and Example 1.40.5, respectively. MS (ESI) m/e 797.4 (M+H)⁺.

### 1.40.9 2-(6-(tert-butoxycarbonyl)-5-(1-((3-(2-((tert-butoxycarbonyl)amino)acetamido)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared using the procedure in Example 1.26.8, replacing Example 1.26.7 with Example 1.40.8. MS (ESI) m/e 783.4 (M+H)⁺.

### 1.40.10 tert-butyl 6-(8-(benzo[d]thiazol-2-ylcarbamoyl)-3,4-dihydroisoquinolin-2(1H)-yl)-3-(1-((3-(2-((tert-butoxycarbonyl)amino)acetamido)-5,7-dimethyladamantan-1-yl)methyl)-5-methyl-1H-pyrazol-4-yl)picolinate

The title compound was prepared using the procedure in Example 1.26.9, replacing Example 1.26.8 with Example 1.40.9. MS (ESI) m/e 915.3 (M+H)⁺.

### 1.40.11 3-(1-{[3-(2-aminoacetamido)-5,7-dimethyltricyclo[3.3.1.1^{3,7}]decan-1-yl]methyl}-5-methyl-1H-pyrazol-4-yl)-6-{8-[(1,3-benzothiazol-2-yl)carbamoyl]-3,4-dihydroisoquinolin-2(1H)-yl}pyridine-2-carboxylic acid

The title compound was prepared using the procedure in Example 1.26.10, replacing Example 1.26.9 with Example 1.40.10. ¹H NMR (400 MHz, dimethyl sulfoxide-d₆) δ 12.82 (s, 1H), 8.00 (dd, 1H), 7.90-7.79 (m, 4H), 7.76 (d, 1H), 7.59 (dd, 1H), 7.49-7.38 (m, 3H), 7.37-7.29 (m, 2H), 7.25 (s, 1H), 6.92 (d, 1H), 4.92 (s, 2H), 3.85 (t, 2H), 3.77 (s, 2H), 3.40 (q, 2H), 2.98 (t, 2H), 2.07 (s, 3H), 1.63 (s, 2H), 1.57-1.38 (m, 4H), 1.15-0.93 (m, 6H), 0.80 (s, 6H). MS (ESI) m/e 759.2 (M+H)⁺.

### 1.41 Synthesis of 3-[1-({3-[(2-aminoethyl)sulfanyl]-5,7-dimethyltricyclo[3.3.1.1^{3,7}]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid

### 1.41.1 3-bromo-5,7-dimethyladamantane-1-carboxylic acid

To a solution of bromine (18.75 mL) was added iron (10.19 g) at 0° C., and the mixture was stirred for 30 minutes. 3,5-Dimethyladamantane-1-carboxylic acid (19 g) was added to the above mixture portionwise. The mixture was stirred at room temperature for 36 hours. After adding ice-water (50 mL) and 6N aqueous HCl (100 mL), the mixture was treated with Na₂SO₃ (100 g dissolved in 500 mL water). The aqueous layer was extracted with dichloromethane (300 mL×4). The combined organic layers were washed with 1N aqueous HCl (300 mL) and brine, dried over magnesium sulfate, filtered and concentrated to give the title compound, which was used in the next step without additional purification. ¹H NMR: (400 MHz, CDCl₃) δ ppm 2.23 (s, 2H), 2.01-1.74 (m, 4H), 1.61-1.47 (m, 6H), 0.93 (s, 6H). LC-MS (ESI) m/e 285.0 (M+H)⁺.

### 1.41.2 3-bromo-5,7-dimethyladamantan-1-yl)methanol

To a solution of Example 1.41.1 (10 g) in tetrahydrofuran (20 mL) was added BH₃.THF (69.6 mL). The mixture was stirred at room temperature for 16 hours. Upon the completion of the reaction, methanol (20 mL) was added dropwise, and the resulting mixture was stirred for 30 minutes. The mixture was concentrated under reduced pressure. The residue was purified by column chromatography on silica gel, eluting with petroleum ether/ethyl acetate (from 8/1 to 5/1), to give the title compound. ¹H NMR: (400 MHz, CDCl₃) δ ppm 3.28 (s, 2H), 1.98-1.95 (m, 6H), 1.38-1.18 (m, 7H), 0.93 (s, 6H).

### 1.41.3 1-((3-bromo-5,7-dimethyladamantan-1-yl)methyl)-1H-pyrazole

A mixture of 2-(tributylphosphoranylidene)acetonitrile (919 mg), 1H-pyrazole (259 mg) and Example 1.41.2 (800 mg) in toluene (8 mL) was stirred at 90° C. for 16 hours. The mixture was concentrated, and the residue was diluted with ethyl acetate (50 mL). The mixture was washed with brine, dried over magnesium sulfate, filtered and concentrated. The residue was purified by silica gel chromatography, eluting with petroleum ether/ethyl acetate, to give the title compound. LC-MS (ESI) m/e 325.1 (M+H)⁺.

### 1.41.4 3-((1H-pyrazol-1-yl)methyl)-5,7-dimethyladamantane-1-thiol

A mixture of Example 1.41.3 (2.8 g) and thiourea (15.82 g) in 33% (w/w) HBr in acetic acid (50 mL) was stirred at 110° C. for 16 hours and concentrated under reduced pressure to give a residue. The residue was dissolved in 20% ethanol in water (v/v: 200 mL), and sodium hydroxide (19.06 g) was added. The resulting solution was stirred at room temperature for 16 hours and concentrated. The residue was dissolved in water (60 mL), and acidified with 6 N aqueous HCl to pH 5-pH 6. The mixture was extracted with ethyl acetate (200 mL×2). The combined organic layers were washed with brine, dried over MgSO₄, filtered and concentrated to give the title compound. MS (ESI) m/e 319.1 (M+H)⁺.

### 1.41.5 2-((-3-((1H-pyrazol-1-yl)methyl)-5,7-dimethyladamantan-1-yl)thio)ethanol

To a solution of Example 1.41.4 (3.3 g) in ethanol (120 mL) was added sodium ethoxide (2.437 g). The mixture was stirred for 10 minutes, and 2-chloroethanol (1.80 mL) was added dropwise. The mixture was stirred at room temperature for 6 hours and neutralized with 1 N aqueous HCl to pH 7. The mixture was concentrated, and the residue was extracted with ethyl acetate (200 mL×2). The combined organic layers were washed with brine, dried over MgSO₄, filtered and concentrated. The residue was purified by column chromatography on silica gel, eluting with petroleum ether/ethyl acetate from 6/1 to 2/1, to give the title compound. MS (ESI) m/e 321.2 (M+H)⁺.

### 1.41.6 2-((-3,5-dimethyl-7-((5-methyl-1H-pyrazol-1-yl)methyl)adamantan-1-yl)thio)ethanol

To a solution of Example 1.41.5 (2.3 g) in tetrahydrofuran (60 mL) was added n-butyllithium (14.35 mL, 2M in hexane) at −20° C. dropwise under nitrogen. The mixture was stirred for 2 hours. Methyl iodide (4.49 mL) was added to the resulting mixture at −20° C., and the mixture was stirred at −20° C. for 2 hours. The reaction was quenched by the dropwise addition of saturated aqueous NH₄Cl solution at −20° C. The resulting mixture was stirred for 10 minutes and acidified with 1 N aqueous HCl to pH 5. The mixture was extracted with ethyl acetate twice. The combined organic layers were washed with brine, dried over MgSO₄, filtered and concentrated to give the title compound. MS (ESI) m/e 335.3 (M+H)⁺.

### 1.41.7 2-((-3-((4-iodo-5-methyl-1H-pyrazol-1-yl)methyl)-5,7-dimethyladamantan-1-yl)thio)ethanol

To a solution of Example 1.41.6 (3.65 g) in N,N-dimethylformamide (90 mL) was added N-iodosuccinimide (3.68 g). The mixture was stirred at room temperature for 16 hours. The reaction was quenched by the addition of ice-water (8 mL) and saturated aqueous NaS₂O₃ solution (8 mL). The mixture was stirred for an additional 10 minutes and extracted with ethyl acetate (30 mL×2). The combined organic layers were washed with brine, dried over MgSO₄, filtered and concentrated under reduced pressure. The residue was purified by silica gel chromatography, eluting with petroleum ether/ethyl acetate (6/1 to 3/1), to give the title compound. MS (ESI) m/e 461.2 (M+H)⁺.

### 1.41.8 di-tert-butyl [2-({3-[(4-iodo-5-methyl-1H-pyrazol-1-yl)methyl]-5,7-dimethyltricyclo[3.3.1.1^{3,7}]decan-1-yl}sulfanyl)ethyl]-2-imidodicarbonate

To a cold solution (0° C. bath) of Example 1.41.7 (3 g) in dichloromethane (100 mL) was added triethylamine (1.181 mL) and mesyl chloride (0.559 mL). The mixture was stirred at room temperature for 4 hours, and the reaction was quenched by the addition of ice-water (30 mL). The mixture was stirred for an additional 10 minutes and was extracted with dichloromethane (50 mL×2). The combined organic layers were washed with brine, dried over MgSO₄, filtered and concentrated under reduced pressure. The residue was dissolved in acetonitrile (100 mL) and NH(Boc)₂ (1.695 g) and Cs₂CO₃ (4.24 g) were added. The mixture was stirred at 85° C. for 16 hours, and the reaction was quenched by the addition of water (20 mL). The mixture was stirred for 10 minutes and was extracted with ethyl acetate (40 mL×2). The combined organic layers were washed with brine, dried over MgSO₄, filtered and concentrated. The residue was purified by silica gel chromatography, eluting with petroleum ether/ethyl acetate from 10/1 to 6/1, to give the title compound. MS (ESI) m/e 660.1 (M+H)⁺.

### 1.41.9 methyl 2-[5-(1-{[3-({2-[bis(tert-butoxycarbonyl)amino]ethyl}sulfanyl)-5,7-dimethyltricyclo[3.3.1.1^{3,7}]decan-1-yl]methyl}-5-methyl-1H-pyrazol-4-yl)-6-(tert-butoxycarbonyl)pyridin-2-yl]-1,2,3,4-tetrahydroisoquinoline-8-carboxylate

The title compound was prepared using the procedure in Example 1.4.7, replacing Example 1.4.6 and Example 1.4.2 with Example 1.40.7 and Example 1.41.8, respectively. LC-MS (ESI) m/e 900.6 (M+H)⁺.

### 1.41.10 2-(6-(tert-butoxycarbonyl)-5-(1-((3-((2-((tert-butoxycarbonyl)amino)ethyl)thio)-5,7-dimethyladamantan-1-yl)methyl)-5-methyl-1H-pyrazol-4-yl)pyridin-2-yl)-1,2,3,4-tetrahydroisoquinoline-8-carboxylic acid

A slurry of lithium hydroxide (553 mg) in water (4.03 mL) and methanol (4 mL) was cooled to 15° C. A solution of Example 1.41.9 (800 mg) in tetrahydrofuran (3.23 mL) and methanol (4 mL) was added slowly, and the reaction was stirred at room temperature. After 18 hours the reaction was cooled in an ice-bath and 1.8 g of phosphoric acid in water (4 mL) was added. The biphasic mixture was transferred to a separatory funnel and extracted with ethyl acetate to give the title compound. LC-MS (ESI) m/e 786.2 (M+H)⁺.

### 1.41.11 tert-butyl 6-(8-(benzo[d]thiazol-2-ylcarbamoyl)-3,4-dihydroisoquinolin-2(1H)-yl)-3-(1-((3-((2-((tert-butoxycarbonyl)amino)ethyl)thio)-5,7-dimethyladamantan-1-yl)methyl)-5-methyl-1H-pyrazol-4-yl)picolinate

A 4 mL amber vial containing Example 1.41.10 (699 mg) was charged with ethyl acetate (5 mL) and 1,1′-carbonyldiimidazole (231 mg) and was stirred for 7 hours at room temperature. A solution of benzo[d]thiazol-2-amine (227 mg) and 1,8-diazabicyclo[5.4.0]undec-7-ene (0.228 mL) in acetonitrile (3 mL) was added, and the reaction was heated to 70° C. After stirring for 18 hours, the reaction was quenched by the addition of 10 mL 1N aqueous HCl and was extracted with ethyl acetate to give the title compound, which was used in the subsequent step without further purification. MS (ESI) m/e 818.2 (M+H)⁺.

### 1.41.12 3-[1-({3-[(2-aminoethyl)sulfanyl]-5,7-dimethyltricyclo[3.3.1.1^{3,7}]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid

To a solution of Example 1.41.11 (510 mg) in dichloromethane (10 mL) was added trifluoroacetic acid (10 mL), and the reaction was stirred at room temperature for 30 minutes. The reaction was quenched with aqueous saturated NaHCO₃ solution and extracted with dichloromethane. The product was purified by reverse-phase HPLC on a Gilson system (C18 column), eluting with 5-80% acetonitrile in water containing 0.1% trifluoroacetic acid, to give the title compound. ¹H NMR (400 MHz, DMSO-d₆) δ ppm 12.86 (bs, 1H), 8.03 (d, 1H), 7.76 (m, 2H), 7.62 (d, 1H), 7.39 (m, 6H), 6.95 (t, 1H), 5.07 (s, 1H), 4.96 (s, 1H), 3.85 (m, 4H), 3.01 (t, 2H), 2.97 (t, 2H), 2.90 (m, 2H), 2.69 (m, 2H), 2.11 (s, 3H), 1.54 (s, 2H), 1.36, (m, 4H), 1.17 (m, 4H), 1.08 (m, 2H), 0.84 (s, 6H). MS (ESI) m/e 762.2 (M+H)⁺.

### 1.42 Synthesis of 3-(1-{[3-(3-aminopropyl)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid

### 1.42.1 1-((3-allyl-5,7-dimethyladamantan-1-yl)methyl)-1H-pyrazole

To a solution of Example 1.41.3 (0.825 g) in toluene (5 mL) was added N, N′-azoisobutyronitrile (AIBN, 0.419 g) and allyltributylstannane (2.039 mL). The mixture was purged with N₂ stream for 15 minutes, heated at 80° C. for 8 hours and concentrated. The residue was purified by silica gel chromatography, eluting with 5% ethyl acetate in petroleum ether, to provide the title compound. MS (ESI) m/e 285.2 (M+H)⁺.

### 1.42.2 1-((3-allyl-5,7-dimethyladamantan-1-yl)methyl)-5-methyl-1H-pyrazole

To a solution of Example 1.42.1 (200 mg) in tetrahydrofuran (5 mL) at −78° C. under N₂ was added n-butyllithium (2.81 mL, 2.5 M in hexane). The mixture was stirred for 2 hours while the temperature increased to −20° C. and was stirred at −20° C. for 1 hour. Iodomethane (0.659 mL) was added, and the resulting mixture was stirred for 0.5 hour at −20° C. The reaction was quenched with saturated aqueous NH₄Cl solution and extracted with ethyl acetate twice. The organic layer was washed with brine to give the title compound. MS (ESI) m/e 299.2 (M+H)⁺.

### 1.42.3 3-(3,5-dimethyl-7-((5-methyl-1H-pyrazol-1-yl)methyl)adamantan-1-yl)propan-1-ol

Under a nitrogen atmosphere, a solution of Example 1.42.2 (2.175 g, 7.29 mmol) in anhydrous tetrahydrofuran (42.5 mL) was cooled to 0° C. BH₃.THF (15.30 mL) was added dropwise. The reaction mixture was stirred at room temperature for 2 hours and cooled to 0° C. To the reaction mixture was added 10 N aqueous NaOH (5.03 mL) dropwise, followed by 30 percent H₂O₂(16.52 mL) water solution. The resulting mixture was warmed to room temperature and stirred for 90 minutes. The reaction was quenched with 10 percent aqueous hydrochloric acid (35 mL). The organic layer was separated, and the aqueous layer was extracted with ethyl acetate (2×60 mL). The combined organic layers were washed with brine (3×60 mL) and cooled in an ice bath. A saturated aqueous solution of sodium sulfite (15 mL) was carefully added and the mixture was stirred for a few minutes. The organic layer was dried over sodium sulfate, filtered, and concentrated in vacuo. The residue was purified by silica gel chromatography, eluting with petroleum ether/ethyl acetate (3:1 to 1:1), to provide the title compound. MS (ESI) m/e 317.3 (M+H)⁺.

### 1.42.4 3-(3-((4-iodo-5-methyl-1H-pyrazol-1-yl)methyl)-5,7-dimethyladamantan-1-yl)propan-1-ol

A mixture of Example 1.42.3 (1.19 g) and 1-iodopyrrolidine-2, 5-dione (1.015 g) in N,N-dimethylformamide (7.5 mL) was stirred for 16 hours at room temperature. The reaction was quenched with saturated aqueous Na₂SO₃ solution The mixture was diluted with ethyl acetate and washed with saturated aqueous Na₂SO₃, saturated aqueous Na₂CO₃ solution, water and brine. The organic layer was dried over anhydrous Na₂SO₄, filtered, and concentrated. The residue was purified by silica gel chromatography, eluting with petroleum ether/ethyl acetate (3:1 to 1:1), to provide the title compound. MS (ESI) m/e 443.1 (M+H)⁺.

### 1.42.5 3-(3-((4-iodo-5-methyl-1H-pyrazol-1-yl)methyl)-5,7-dimethyladamantan-1-yl)propyl methanesulfonate

To a solution of Example 1.42.4 (1.55 g, 3.50 mmol) in dichloromethane (20 mL) at 0° C. were added triethylamine (0.693 mL) and mesyl chloride (0.374 mL) slowly. The mixture was stirred for 3.5 hours at 20° C. and was diluted with dichloromethane. The organic layer was washed with saturated aqueous NH₄Cl, saturated aqueous NaHCO₃ solution and brine. The organic layer was dried over Na₂SO₄, filtered and concentrated to provide the title compound. MS (ESI) m/e 521.1 (M+H)⁺.

### 1.42.6 di-tert-butyl (3-{3-[(4-iodo-5-methyl-1H-pyrazol-1-yl)methyl]-5,7-dimethyltricyclo[3.3.1.1^{3,7}]decan-1-yl}propyl)-2-imidodicarbonate

To a solution of Example 1.42.5 (1.92 g) in acetonitrile (40 mL) at 20° C. were added di-tert-butyl iminodicarbonate (0.962 g) and Cs₂CO₃ (2.404 g). The mixture was stirred for 16 hours at 80° C. and diluted with ethyl acetate, washed with water and brine. The organic layer was dried over Na₂SO₄, filtered and concentrated. The residue was purified by silica gel chromatography, eluting with petroleum ether/ethyl acetate (10:1), to provide the title compound. MS (ESI) m/e 642.3 (M+H)⁺.

### 1.42.7 methyl 2-[5-{1-[(3-{3-[bis(tert-butoxycarbonyl)amino]propyl}-5,7-dimethyltricyclo[3.3.1.1^{3,7}]decan-1-yl)methyl]-5-methyl-1H-pyrazol-4-yl}-6-(tert-butoxycarbonyl)pyridin-2-yl]-1,2,3,4-tetrahydroisoquinoline-8-carboxylate

The title compound was prepared using the procedure in Example 1.4.7, replacing Example 1.4.6 and Example 1.4.2 with Example 1.40.7 and Example 1.42.6, respectively. LC-MS (ESI) m/e 882.6 (M+H)⁺.

### 1.42.8 2-(6-(tert-butoxycarbonyl)-5-(1-((3-(3-((tert-butoxycarbonyl)amino)propyl)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared using the procedure in Example 1.41.10 substituting Example 1.42.7 for Example 1.41.9. LC-MS (ESI) m/e 468.5 (M+H)⁺.

### 1.42.9 tert-butyl 6-(8-(benzo[d]thiazol-2-ylcarbamoyl)-3,4-dihydroisoquinolin-2(1H)-yl)-3-(1-((3-(3-((tert-butoxycarbonyl)amino)propyl)-5,7-dimethyladamantan-1-yl)methyl)-5-methyl-1H-pyrazol-4-yl)picolinate

The title compound was prepared using the procedure in Example 1.41.11 substituting Example 1.42.8 for Example 1.41.10.

### 1.42.10 3-(1-{[3-(3-aminopropyl)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid

The title compound was prepared using the procedure in Example 1.41.12 substituting Example 1.42.9 for Example 1.41.11. ¹H NMR (500 MHz, DMSO-d) δ ppm 12.86 (s, 1H), 8.03 (d, 1H), 7.79 (d, 1H), 7.62 (d, 4H), 7.47 (dt, 3H), 7.36 (q, 2H), 7.27 (s, 1H), 6.95 (d, 1H), 4.95 (s, 2H), 3.77 (s, 2H), 3.01 (t, 2H), 2.72 (q, 2H), 2.09 (s, 3H), 1.45 (t, 2H), 1.18-1.05 (m, 9H), 1.00 (d, 6H), 0.80 (s, 6H). MS (ESI) m/e 468.5 (M+H)⁺.

### 1.43 3-(1-{[3-(2-aminoethoxy)-5,7-dimethyltricyclo[3.3.1.1^{3,7}]decan-1-yl]methyl}-5-methyl-1H-pyrazol-4-yl)-6-{5-[(1,3-benzothiazol-2-yl)carbamoyl]quinolin-3-yl}pyridine-2-carboxylic acid

### 1.43.1 methyl 3-bromoquinoline-5-carboxylate

To a solution of 3-bromoquinoline-5-carboxylic acid (2 g) in methanol (30 mL) was added concentrated H₂SO₄ (5 mL). The solution was stirred at reflux overnight. The mixture was concentrated under reduced pressure. The residue was dissolved in ethyl acetate (300 mL) and washed with aqueous Na₂CO₃ solution, water and brine. After drying over anhydrous sodium sulfate, filtration and evaporation of the solvent gave the title product. MS (ESI) m/e 266 (M+H)⁺.

### 1.43.2 methyl 3-(4,4,5,5-tetramethyl-1,3,2-dioxaborolan-2-yl)quinoline-5-carboxylate

To a solution of Example 1.43.1 (356 mg) in N,N-dimethylformamide (5 mL) was added [1,1′-bis(diphenylphosphino)ferrocene]dichloropalladium(II) (55 mg), potassium acetate (197 mg) and bis(pinacolato)diboron (510 mg). The mixture was stirred at 60° C. overnight. The mixture was cooled to room temperature and used in the next reaction without further work up. MS (ESI) m/e 339.2 (M+Na)⁺.

### 1.43.3 methyl 3-[5-{1-[(3-{2-[bis(tert-butoxycarbonyl)amino]ethoxy}-5,7-dimethyltricyclo[3.3.1.1^{3,7}]decan-1-yl)methyl]-5-methyl-1H-pyrazol-4-yl}-6-(tert-butoxycarbonyl)pyridin-2-yl]quinoline-5-carboxylate

To a solution of Example 1.43.2 (626 mg) in 1,4-dioxane (10 mL) and water (5 mL) was added Example 1.23.3 (1.46 g), bis(triphenylphosphine)palladium(II) dichloride (140 mg), and CsF (911 mg). The mixture was stirred at 120° C. for 30 minutes under microwave conditions (Biotage Initiator). The mixture was diluted with ethyl acetate (200 mL), washed with water and brine, dried over anhydrous sodium sulfate, filtered and concentrated. The residue was purified by silica gel chromatography, eluting with 20% ethyl acetate in heptane (1 L) to give the title product. MS (ESI) m/e 880.3 (M+H)⁺.

### 1.43.4 3-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)quinoline-5-carboxylic acid

To a solution of Example 1.43.3 (1.34 g) in tetrahydrofuran (10 mL), methanol (5 mL) and water (5 mL) was added LiOH H₂O (120 mg), and the mixture was stirred at room temperature overnight. The mixture was acidified with 2N aqueous HCl, diluted with ethyl acetate (400 mL), washed with water and brine and dried over anhydrous sodium sulfate. Filtration and evaporation of solvent gave the title product. MS (APCI) m/e 766.3 (M+H)⁺.

### 1.43.5 3-(1-{[3-(2-aminoethoxy)-5,7-dimethyltricyclo[3.3.1.1^{3,7}]decan-1-yl]methyl}-5-methyl-1H-pyrazol-4-yl)-6-{5-[(1,3-benzothiazol-2-yl)carbamoyl]quinolin-3-yl}pyridine-2-carboxylic acid

To a solution of Example 1.43.4 (200 mg) in dichloromethane (10 mL) was added benzo[d]thiazol-2-amine (39.2 mg), 1-ethyl-3-[3-(dimethylamino)propyl]-carbodiimide hydrochloride (50 mg) and 4-dimethylaminopyridine (32 mg). The mixture was stirred at room temperature overnight. The reaction mixture was diluted with ethyl acetate (200 mL), washed with water and brine, dried over anhydrous sodium sulfate, filtered and concentrated. The residue was dissolved in dichloromethane and trifluoroacetic acid (10 mL, 1:1), and the reaction was stirred overnight. The mixture was concentrated, and the residue was dissolved in N,N-dimethylformamide (12 mL) and purified by reverse-phase HPLC on a Gilson system (C18 column), eluting with 20-80% acetonitrile in water containing 0.1% trifluoroacetic acid, to give the title product. MS (ESI) m/e 742.1 (M+H)⁺.

### Example 2. Synthesis of Exemplary Synthons

This example provides synthetic methods for exemplary synthons useful more making ADCs.

### 2.1. Synthesis of N-[6-(2,5-dioxo-2,5-dihydro-1H-pyrrol-1-yl)hexanoyl]-L-valyl-N-{4-[({[2-({3-[(4-{6-[1-(1,3-benzothiazol-2-ylcarbamoyl)-1,2,3,4-tetrahydroquinolin-7-yl]-2-carboxypyridine-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide (Synthon BS)

Example 1.1.14 (72 mg) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (91 mg) in N,N-dimethylformamide (3 mL) was cooled in a water-ice bath and N,N-diisopropylethylamine (0.12 mL) was added. The mixture was stirred at 0° C. for 2 hours and acetic acid (0.057 mL) was added. After concentration of the solvents, the residue was purified via HPLC (20-80% acetonitrile in 0.1% TFA/water) to provide the title compound. ¹H NMR (400 MHz, dimethyl sulfoxide-d₆) δ ppm 9.98 (s, 1H), 8.40 (s, 1H), 8.06 (d, 1H), 8.00 (d, 1H), 7.74-7.89 (m, 4H), 7.59 (d, 2H), 7.46 (s, 2H), 7.37 (t, 1H), 7.18-7.32 (m, 4H), 6.99 (s, 2H), 6.01 (s, 1H), 4.98 (s, 3H), 4.38 (d, 2H), 3.47 (d, 2H), 3.36 (t, 2H), 3.28 (t, 2H), 2.91-3.10 (m, 2H), 2.79-2.91 (m, 4H), 2.19-2.25 (m, 3H), 2.06-2.20 (m, 2H), 1.89-2.02 (m, 3H), 1.53-1.74 (m, 2H), 1.30-1.55 (m, 8H), 1.06-1.29 (m, 10H), 0.91-1.06 (m, 2H), 0.76-0.89 (m, 12H). MS (ESI) m/e 1356.3 (M+H)⁺.

### 2.2. Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide (Synthon DK)

To a solution of 4-((S)-2-((S)-2-(6-(2,5-dioxo-2,5-dihydro-1H-pyrrol-1-yl)hexanamido)-3-methylbutanamido)-5-ureidopentanamido)benzyl 4-nitrophenyl carbonate (57 mg) and Example 1.2.2 (57 mg) in N,N-dimethylformamide (6 mL) was added N,N-diisopropylethylamine (0.5 mL). The mixture was stirred overnight. The mixture was concentrated under vacuum and the residue was diluted with methanol (3 mL) and acetic acid (0.3 mL), loaded onto a 300 g reverse-phase column, and eluted with 30-70% acetonitrile in 0.1% aqueous TFA solution to provide the title compound. ¹H NMR (400 MHz, dimethyl sulfoxide-d₆) δ ppm 9.97 (s, 1H), 8.73 (d, 1H), 8.07 (d, 1H), 7.90-7.98 (m, 1H), 7.71-7.87 (m, 4H), 7.54-7.63 (m, 2H), 7.45 (d, 1H), 7.32-7.42 (m, 2H), 7.17-7.31 (m, 3H), 6.92-7.03 (m, 3H), 5.88-6.08 (m, 1H), 4.97 (s, 3H), 4.29-4.46 (m, 4H), 4.12-4.26 (m, 4H), 3.86 (s, 3H), 3.21-3.41 (m, 8H), 2.78-3.10 (m, 6H), 2.20 (s, 3H), 1.90-2.18 (m, 3H), 0.92-1.77 (m, 24H), 0.75-0.88 (m, 6H). MS (ESI) m/e 1360.2 (M+H)⁺.

### 2.3. Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)-1-methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide (Synthon DQ)

The title compound was prepared by substituting Example 1.3.2 for Example 1.2.2 in Example 2.2. ¹H NMR (500 MHz, dimethyl sulfoxide-d₆) δ ppm 9.99 (s, 1H), 8.17-8.35 (m, 1H), 8.07 (d, 1H), 7.89 (d, 1H), 7.71-7.84 (m, 4H), 7.55-7.65 (m, 2H), 7.43 (s, 1H), 7.36 (t, 1H), 7.28 (d, 2H), 7.21 (t, 1H), 6.99 (s, 2H), 6.83 (d, 1H), 5.97 (s, 1H), 5.28-5.51 (m, 2H), 4.98 (s, 2H), 4.32-4.44 (m, 1H), 4.19 (dd, 1H), 3.97-4.13 (m, 2H), 3.85 (s, 2H), 3.29 (d, 3H), 3.00 (s, 3H), 2.80-2.98 (m, 4H), 2.18-2.26 (m, 3H), 1.88-2.17 (m, 3H), 0.91-1.73 (m, 23H), 0.74-0.92 (m, 12H). MS (ESI) m/e 1373.3 (M+H)⁺.

### 2.4. Synthesis of 4-[(1E)-3-({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon DJ)

### 2.4.1. (E)-tert-butyldimethyl((3-(4,4,5,5-tetramethyl-1,3,2-dioxaborolan-2-yl)allyl)oxy)silane

To a flask charged with tert-butyldimethyl(prop-2-yn-1-yloxy)silane (5 g) and dichloromethane (14.7 mL) under nitrogen atmosphere was added dropwise 4,4,5,5-tetramethyl-1,3,2-dioxaborolane (3.94 g). The mixture was stirred at room temperature for one minute then transferred via cannula to a nitrogen-sparged flask containing Cp₂ZrClH (chloridobis(η5-cyclopentadienyl)hydridozirconium, Schwartz's Reagent) (379 mg). The resulting reaction mixture was stirred at room temperature for 16 hours. The mixture was carefully quenched with water (15 mL), and then extracted with diethyl ether (3×30 mL). The combined organic phases were washed with water (15 mL), dried over MgSO₄, filtered, concentrated and purified by silica gel chromatography, eluting with a gradient from 0-8% ethyl acetatate/heptanes to give the title compound. MS (ESI) m/z 316.0 (M+NH₄)⁺.

### 2.4.2. (2S,3R,4S,5S,6S)-2-(4-bromo-2-nitrophenoxy)-6-(methoxycarbonyl)tetrahydro-2H-pyran-3,4,5-triyl triacetate

(2R,3R,4S,5S,6S)-2-Bromo-6-(methoxycarbonyl)tetrahydro-2H-pyran-3,4,5-triyl triacetate (5 g) was dissolved in acetonitrile (100 mL). Ag₂O (2.92 g) was added to the solution and the reaction was stirred for 5 minutes at room temperature. 4-Bromo-2-nitrophenol (2.74 g) was added and the reaction mixture was stirred at room temperature for 4 hours. The silver salt residue was filtered through diatomaceous earth and the filtrate was concentrated under reduced pressure. The residue was purified by silica gel chromatography eluting with a gradient of 10-70% ethyl acetate in heptanes to provide the title compound. MS (ESI+) m/z 550.9 (M+NH₄)⁺.

### 2.4.3. (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate

Example 2.4.2 (1 g), sodium carbonate (0.595 g), tris(dibenzylideneacetone)dipalladium (Pd₂(dba)₃) (0.086 g), and 1,3,5,7-tetramethyl-6-phenyl-2,4,8-trioxa-6-phosphaadamantane (0.055 g) were combined in a 3-neck 50-mL round bottom flask equipped with a reflux condenser and the system was degassed with nitrogen. Separately, a solution of Example 2.4.1 (0.726 g) in tetrahydrofuran (15 mL) was degassed with nitrogen for 30 minutes. This latter solution was transferred via cannula into the flask containing the solid reagents, followed by addition of degassed water (3 mL) via syringe. The reaction was heated to 60° C. for two hours. The reaction mixture was partitioned between ethyl acetate (3×30 mL) and water (30 mL). The combined organic phases were dried (Na₂SO₄), filtered, and concentrated. The residue was purified by silica gel chromatography, eluting with a gradient from 0-35% ethyl acetate/heptanes to provide the title compound. MS (ESI+) m/z 643.1 (M+NH₄)⁺.

### 2.4.4. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate

A 500-mL three-neck, nitrogen-flushed flask equipped with a pressure-equalizing addition funnel was charged with zinc dust (8.77 g). A degassed solution of Example 2.4.3 (8.39 g) in tetrahydrofuran (67 mL) was added via cannula. The resulting suspension was chilled in an ice bath and then 6N aqueous HCl (22.3 mL) was added dropwise via addition funnel at such a rate that the internal temperature of the reaction did not exceed 35° C. After the addition was complete, the reaction mixture was stirred for two hours at room temperature and then filtered through a pad of diatomaceous earth, rinsing with water and ethyl acetate. The filtrate was treated with saturated aqueous NaHCO₃ solution until the water layer was no longer acidic, and the mixture was filtered to remove the resulting solids. The filtrate was transferred to a separatory funnel and the layers were separated. The aqueous layer was extracted with ethyl acetate (3×75 mL) and the combined organic layers were washed with water (100 mL), dried over Na₂SO₄, filtered, and concentrated. The residue was triturated with diethyl ether and the solid was collected by filtration to give the title compound. MS (ESI+) m/z 482.0 (M+H)⁺.

### 2.4.5. (9H-fluoren-9-yl)methyl (3-chloro-3-oxopropyl)carbamate

To a solution of 3-((((9H-fluoren-9-yl)methoxy)carbonyl)amino)propanoic acid (5.0 g) in dichloromethane (53.5 mL) was added sulfurous dichloride (0.703 mL). The mixture was stirred at 60° C. for one hour. The mixture was cooled and concentrated to provide the title compound which was used in the next step without further purification.

### 2.4.6. (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate

Example 2.4.4 (6.78 g) was dissolved in dichloromethane (50 mL) and the solution was chilled to 0° C. in an ice bath. N,N-Diisopropylethylamine (3.64 g) was added, followed by dropwise addition of a solution of Example 2.4.5 (4.88 g) in dichloromethane (50 mL). The reaction was stirred for 16 hours allowing the ice bath to come to room temperature. Saturated aqueous NaHCO₃ solution (100 mL) was added and the layers were separated. The aqueous layer was further extracted with dichloromethane (2×50 mL). The extracts were dried over Na₂SO₄, filtered, concentrated and then purified by silica gel chromatography, eluting with a gradient of 5-95% ethyl acetate/heptane, to give an inseparable mixture of starting aniline and desired title compound. This mixture was partitioned between 1N aqueous HCl (40 mL) and a 1:1 mixture of diethyl ether and ethyl acetate (40 mL), and then the aqueous phase was further extracted with ethyl acetate (2×25 mL). The organic phases were combined, washed with water (2×25 mL), dried over Na₂SO₄, filtered, and concentrated to give the title compound. MS (ESI+) m/z 774.9 (M+H)⁺.

### 2.4.7. (2S,3R,4S,5S,6S)-2-(2-(3-((((9H-fluoren-9-yl)methoxy)carbonyl)amino)propanamido)-4-((E)-3-(((4-nitrophenoxy)carbonyl)oxy)prop-1-en-1-yl)phenoxy)-6-(methoxycarbonyl)tetrahydro-2H-pyran-3,4,5-triyl triacetate

Example 2.4.6 (3.57 g) was dissolved in dichloromethane (45 mL) and bis(4-nitrophenyl)carbonate (2.80 g) was added, followed by dropwise addition of N,N-diisopropylethylamine (0.896 g). The reaction was stirred at room temperature for two hours. Silica gel (20 g) was then added to the reaction solution and the mixture was concentrated to dryness under reduced pressure, keeping the bath temperature at or below 25° C. The silica residue was loaded atop a column and the crude material was purified by silica gel chromatography, eluting with a gradient from 0-100% ethyl acetate-heptane, providing partially purified title compound which was contaminated with nitrophenol. This material was triturated with methyl tert-butyl ether (250 mL) and the resulting slurry was allowed to sit for 1 hour. The title compound was collected by filtration. Three successive crops were collected in a similar fashion to give the title compound. MS (ESI+) m/z 939.8 (M+H)⁺.

### 2.4.8. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

Example 1.1.14 (31 mg) and Example 2.4.7 (33.3 mg) in N,N-dimethylformamide (3 mL) at 0° C. was added N,N-diisopropylethylamine (25 μL). The mixture was stirred overnight, diluted with ethyl acetate and washed with water and brine. The organic layer was dried over Na₂SO₄, filtered, and concentrated. The residue was dissolved in methanol (2 mL) and tetrahydrofuran (1 mL), cooled to 0° C., and 3 M lithium hydroxide aqueous solution (0.35 mL) was added. The mixture was stirred at 0° C. for 4 hours, concentrated and purified by a Gilson HPLC system (C18 column), eluting with 0-60% acetonitrile in 0.1% TFA/water to provide the title compound.

### 2.4.9. 4-[(1E)-3-({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

To a solution of Example 2.4.8 (19 mg) in N,N-dimethylformamide (2.5 mL) at 0° C. was added 2,5-dioxopyrrolidin-1-yl 6-(2,5-dioxo-2,5-dihydro-1H-pyrrol-1-yl)hexanoate (10 mg) and N,N-diisopropylethylamine (11.08 μL). The mixture was stirred at 0° C. for 15 minutes and a few drops of acetic acid were added. The mixture was purified by a Gilson HPLC system (C18 column), eluting with 20-60% acetonitrile in 0.1% TFA/water to provide the title compound. ¹H NMR (500 MHz, dimethyl sulfoxide-4) δ ppm 9.03 (s, 1H), 8.40 (s, 1H), 8.25 (d, 1H), 8.00 (d, 1H), 7.73-7.91 (m, 4H), 7.46 (s, 2H), 7.37 (t, 1H), 7.29 (d, 1H), 7.22 (t, 1H), 7.08-7.13 (m, 1H), 7.04 (d, 1H), 6.98 (s, 2H), 6.56 (d, 1H), 6.10-6.25 (m, 1H), 4.86 (s, 1H), 4.64 (d, 2H), 3.95 (d, 2H), 3.86 (d, 4H), 3.24-3.41 (m, 4H), 2.79-2.96 (m, 6H), 2.54 (t, 2H), 2.21 (s, 3H), 2.03 (t, 2H), 1.90-1.98 (m, 2H), 1.34-1.52 (m, 6H), 1.20-1.30 (m, 5H), 0.89-1.20 (m, 8H), 0.82 (d, 6H). MS (ESI) m/e 1391.2 (M+H)⁺.

### 2.5. Synthesis of 4-[(1E)-3-({[2-({3-[(4-{6-[4-(1,3-benzothiazol-2-ylcarbamoyl)-1-methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon DO)

### 2.5.1. 3-(1-((3-(2-((E)-4-(3-(3-((((9H-fluoren-9-yl)methoxy)carbonyl)amino)propanamido)-4-(((2S,3R,4S,5S,6S)-3,4,5-triacetoxy-6-(methoxycarbonyl)tetrahydro-2H-pyran-2-yl)oxy)phenyl)-N-methylbut-3-enamido)ethoxy)-5,7-dimethyladamantan-1-yl)methyl)-5-methyl-1H-pyrazol-4-yl)-6-(4-(benzo[d]thiazol-2-ylcarbamoyl)-1-methyl-1,2,3,4-tetrahydroquinoxalin-6-yl)picolinic acid

To a cold (0° C.) solution of Example 2.4.7 (98 mg) and Example 1.3.2 (91 mg) was added N-ethyl-N-isopropylpropan-2-amine (0.054 mL). The reaction was slowly warmed to room temperature and stirred overnight. The reaction was quenched by the addition of water and ethyl acetate. The layers were separated, and the aqueous was extracted with additional ethyl acetate (2×). The combined organics were dried with anhydrous sodium sulfate, filtered and concentrated under reduced pressure. The residue was used in the subsequent step without further purification. MS (ESI) m/e 1576.8 (M+H)⁺.

### 2.5.2. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(4-(benzo[d]thiazol-2-ylcarbamoyl)-1-methyl-1,2,3,4-tetrahydroquinoxalin-6-yl)picolinic acid

To a solution of Example 2.5.1 (158 mg) in tetrahydrofuran/methanol/water (2:1:1, 4 mL) was added lithium hydroxide monohydrate (20 mg). The reaction mixture was stirred overnight. The mixture was concentrated under vacuum, acidified with TFA, and dissolved in dimethyl sulfoxide/methanol (9 mL) and loaded on an HPLC (Gilson system, eluting with 10-85% acetonitrile in 0.1% TFA in water) for purification to give the pure title compound. MS (ESI) m/e 1228.2 (M+NH₄)⁺.

### 2.5.3. 4-[(1E)-3-({[2-({3-[(4-{6-[4-(1,3-benzothiazol-2-ylcarbamoyl)-1-methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

To a solution of Example 2.5.2 (20 mg) and 2,5-dioxopyrrolidin-1-yl 6-(2,5-dioxo-2,5-dihydro-1H-pyrrol-1-yl)hexanoate (6.5 mg) in N,N-dimethylformamide (2 mL) was added N,N-diisopropylethylamine (0.054 mL). The reaction was stirred overnight. The reaction mixture was diluted with methanol (2 mL) and acidified with TFA. The mixture was concentrated and purified on HPLC (Gilson system, eluting with 10-85% acetonitrile in 0.1% TFA in water) to give the pure title compound. ¹H NMR (500 MHz, dimethyl sulfoxide-d₆) δ ppm 9.03 (s, 1H), 8.25 (s, 2H), 7.85-7.95 (m, 2H), 7.72-7.83 (m, 3H), 7.43 (s, 2H), 7.32-7.37 (m, 1H), 7.17-7.25 (m, 1H), 7.08-7.14 (m, 1H), 7.04 (d, 1H), 6.98 (s, 2H), 6.82 (d, 1H), 6.56 (d, 1H), 6.08-6.25 (m, 1H), 4.82-4.92 (m, 1H), 4.64 (d, 3H), 4.00-4.11 (m, 4H), 3.81-3.94 (m, 6H), 3.27-3.50 (m, 17H), 3.00 (s, 3H), 2.83-2.96 (m, 3H), 2.53-2.59 (m, 2H), 2.20 (s, 3H), 2.03 (t, 2H), 1.37-1.55 (m, 4H), 0.90-1.29 (m, 10H), 0.82 (d, 6H). MS (ESI) m/e 1406.2 (M+H)⁺.

### 2.6. Synthesis of 4-[(1E)-3-({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon DP)

### 2.6.1. 3-(1-((3-(2-((E)-4-(3-(3-((((9H-fluoren-9-yl)methoxy)carbonyl)amino)propanamido)-4-(((2S,3R,4S,5S,6S)-3,4,5-triacetoxy-6-(methoxycarbonyl)tetrahydro-2H-pyran-2-yl)oxy)phenyl)-N-methylbut-3-enamido)ethoxy)-5,7-dimethyladamantan-1-yl)methyl)-5-methyl-1H-pyrazol-4-yl)-6-(4-(benzo[d]thiazol-2-ylcarbamoyl)-3,4-dihydro-2H-benzo[b][1,4]oxazin-6-yl)picolinic acid

To a cold (0° C.) solution of Example 2.4.7 (98 mg) and Example 1.2.2 (91 mg) was added N-ethyl-N-isopropylpropan-2-amine (0.054 mL). The reaction was slowly warmed to room temperature and was stirred overnight. The reaction was quenched by the addition of water and ethyl acetate. The layers were separated, and the aqueous layer was extracted twice with additional ethyl acetate. The combined organics were dried with anhydrous sodium sulfate, filtered and concentrated under reduced pressure. The residue was used in the subsequent step without further purification. MS (ESI) m/e 1547.7 (M+H)⁺.

### 2.6.2. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(4-(benzo[d]thiazol-2-ylcarbamoyl)-3,4-dihydro-2H-benzo[b][1,4]oxazin-6-yl)picolinic acid

The title compound was prepared by substituting Example 2.6.1 for Example 2.5.1 in Example 2.5.2. MS (ESI) m/e 1200.1 (M+NH₄)⁺.

### 2.6.3. 4-[(1E)-3-({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

The title compound was prepared by substituting Example 2.6.2 for Example 2.5.2 in Example 2.5.3. ¹H NMR (500 MHz, dimethyl sulfoxide-d₆) δ ppm 9.04 (s, 1H), 8.74 (s, 1H), 8.26 (s, 1H), 7.96 (d, 1H), 7.71-7.92 (m, 4H), 7.35-7.48 (m, 3H), 7.23 (t, 1H), 7.11 (d, 1H), 6.96-7.07 (m, 4H), 6.57 (d, 1H), 6.11-6.24 (m, 1H), 4.81-4.93 (m, 1H), 4.65 (d, 2H), 4.32-4.40 (m, 2H), 4.17 (s, 3H), 3.23-3.51 (m, 14H), 2.83-2.98 (m, 3H), 2.54 (t, 2H), 2.21 (s, 3H), 2.03 (t, 2H), 1.34-1.55 (m, 6H), 0.92-1.31 (m, 13H), 0.82 (d, 6H). MS (ESI) m/e 1415.2 (M+Na)⁺.

### 2.7. Synthesis of 4-[(1E)-3-({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon HO)

### 2.7.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a cold (0° C.) solution of Example 2.4.7 (22 mg) and Example 1.6.3 (20 mg) was added N-ethyl-N-isopropylpropan-2-amine (0.054 mL). The reaction was slowly warmed to room temperature and stirred overnight. The reaction was quenched by the addition of water and ethyl acetate. The layers were separated, and the aqueous layer was extracted twice with additional ethyl acetate. The combined organics were dried with anhydrous sodium sulfate, filtered and concentrated under reduced pressure to give the crude title compound which was dissolved in tetrahydrofuran/methanol/water (2:1:1, 4 mL). Lithium hydroxide monohydrate (40 mg) was added, and the reaction mixture stirred overnight. The mixture was then concentrated under vacuum, acidified with TFA, dissolved in dimethyl sulfoxide/methanol and purified on an HPLC (Gilson system, eluting with 10-85% acetonitrile in 0.1% TFA in water) to give the title compound.

### 2.7.2. 4-[(1E)-3-({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

The title compound was prepared by substituting Example 2.7.1 for Example 2.5.2 in Example 2.5.3. ¹H NMR (400 MHz, dimethyl sulfoxide-d₆) δ ppm 13.09 (s, 1H), 9.02 (s, 2H), 8.37 (d, 1H), 8.12-8.29 (m, 4H), 8.06 (s, 1H), 8.02 (d, 1H), 7.93 (d, 1H), 7.76-7.89 (m, 2H), 7.70 (t, 1H), 7.43-7.54 (m, 2H), 7.37 (t, 1H), 7.00-7.13 (m, 2H), 6.98 (s, 2H), 6.56 (d, 1H), 6.08-6.25 (m, 1H), 4.86 (s, 1H), 4.64 (d, 2H), 3.81-3.94 (m, 6H), 3.18-3.51 (m, 12H), 2.78-2.96 (m, 4H), 2.49-2.59 (m, 2H), 2.22 (s, 3H), 2.03 (t, 2H), 1.33-1.54 (m, 6H), 0.93-1.30 (m, 12H), 0.82 (d, 6H). MS (ESI) m/e 1408.3 (M+Na)⁺.

### 2.8. Synthesis of 4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1˜3,7˜]dec-1-yl}oxy)ethyl](oxetan-3-yl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon IT)

### 2.8.1. 3-(1-(((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(oxetan-3-yl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid, Trifluoroacetic Acid

To a solution of Example 1.16.7 (0.039 g) and Example 2.4.7 (0.048 g) in N,N-dimethylformamide (1 mL) was added N,N-diisopropylethylamine (0.037 mL), and the reaction was stirred at room temperature for 2 days. The reaction was concentrated, the residue was re-dissolved in a mixture of methanol (0.5 mL) and tetrahydrofuran (0.5 mL) and treated with lithium hydroxide monohydrate (0.027 g) in water (0.5 mL), and the solution was stirred at room temperature. After stirring for 1 hour, the reaction was quenched with trifluoroacetic acid (0.066 mL), diluted with N,N-dimethylformamide (1 mL), and purified by HPLC using a Gilson system eluting with 10-60% acetonitrile in water containing 0.1% v/v trifluoroacetic acid. The desired fractions were combined and freeze-dried to provide the title compound.

### 2.8.2. 4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](oxetan-3-yl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

To a solution of Example 2.8.1 (0.024 g) and 2,5-dioxopyrrolidin-1-yl 6-(2,5-dioxo-2,5-dihydro-1H-pyrrol-1-yl)hexanoate (8.95 mg) in N,N-dimethylformamide (0.5 mL) was added N-ethyl-N-isopropylpropan-2-amine (0.017 mL), and the reaction was stirred at room temperature for 2 hours. The reaction was diluted with N,N-dimethylformamide (1 mL) and water (1 mL) and was purified by HPLC using a Gilson system eluting with 10-60% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.83 (s, 1H), 9.02 (s, 1H), 8.22 (d, 1H), 8.02 (d, 1H), 7.86 (t, 1H), 7.78 (d, 1H), 7.60 (d, 1H), 7.56-7.39 (m, 3H), 7.39-7.30 (m, 2H), 7.27 (s, 1H), 7.14-6.89 (m, 5H), 6.56 (d, 1H), 4.94 (s, 2H), 4.83 (t, 1H), 4.63 (t, 2H), 4.54 (t, 1H), 3.93-3.83 (m, 6H), 3.83-3.75 (m, 4H), 3.33 (dt, 10H), 2.99 (t, 2H), 2.54 (d, 2H), 2.08 (d, 3H), 2.02 (t, 2H), 1.54-0.72 (m, 26H). MS (ESI) m/e 1433.3 (M+H)⁺.

### 2.9. Synthesis of 4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon KA)

### 2.9.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (150 mg) was dissolved in N,N-dimethylformamide (0.5 mL), and Example 2.4.7 (190 mg) and N-ethyl-N-isopropylpropan-2-amine (0.30 mL) was added. The reaction was stirred at room temperature overnight. Additional Example 2.4.7 (70 mg) and N,N-diisopropylethylamine (0.10 mL) were added and the reaction was allowed to stir another day. The reaction was then concentrated and the residue was dissolved in tetrahydrofuran (2 mL) and methanol (2 mL), then 1.94N aqueous lithium hydroxide monohydrate (1.0 mL) was added and the mixture was stirred at room temperature for one hour. Purification by reverse phase chromatography (C18 column), eluting with 10-90% acetonitrile in 0.1% TFA/water, provided the title compound as a trifluoroacetic acid salt. MS (ESI) m/e 1270.4 (M−H)⁻.

### 2.9.2. 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-(6-(2,5-dioxo-2,5-dihydro-1H-pyrrol-1-yl)hexanamido)propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

Example 2.9.1 (16 mg) was dissolved in N,N-dimethylformamide (0.3 mL), then 2,5-dioxopyrrolidin-1-yl 6-(2,5-dioxo-2,5-dihydro-1H-pyrrol-1-yl)hexanoate (5 mg) and N-ethyl-N-isopropylpropan-2-amine (11 μL) were added. The reaction mixture was stirred for three hours at room temperature, and purification by reverse phase chromatography (C18 column), eluting with 10-90% acetonitrile in 0.1% TFA/water, provided the title compound. H NMR (400 MHz, dimethyl sulfoxide-d₆) δ ppm 12.71 (v br s, 1H), 9.03 (s, 1H), 8.25 (s, 1H), 8.01 (d, 1H), 7.87 (br m, 1H), 7.76 (t, 2H), 7.50 (d, 1H), 7.46 (t, 1H), 7.33 (t, 1H), 7.28 (s, 1H), 7.08 (d, 1H), 7.03 (m, 2H), 6.98 (s, 2H), 6.56 (d, 1H), 6.17 (m, 1H), 5.00 (s, 2H), 4.86 (br m, 1H), 4.64 (d, 2H), 3.88 (m, 6H), 3.79 (br m, 2H), 3.43, 3.35 (m, m, total 16H), 3.22 (s, 3H), 2.80 (m, 2H), 2.54 (m, 2H), 2.09 (s, 3H), 2.03 (t, 2H), 1.45 (m, 6H), 1.37 (br m, 2H), 1.28-0.90 (m, 10H), 0.77-0.82 (m, 6H). MS (ESI) m/e 1463.5 (M−H)⁻.

### 2.10. Synthesis of 4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-({N-[(2,5-dioxo-2,5-dihydro-1H-pyrrol-1-yl)acetyl]-beta-alanyl}amino)phenyl beta-D-glucopyranosiduronic acid (Synthon KB)

Example 2.9.1 (16 mg) was dissolved in N,N-dimethylformamide (0.3 mL), then 2,5-dioxopyrrolidin-1-yl 2-(2,5-dioxo-2,5-dihydro-1H-pyrrol-1-yl)acetate (4 mg) and N-ethyl-N-isopropylpropan-2-amine (11 μL) were added. The reaction mixture was stirred for three hours at room temperature, and purification by reverse phase chromatography (C18 column), eluting with 10-90% acetonitrile in 0.1% TFA/water, provided the title compound. ¹H NMR (400 MHz, dimethyl sulfoxide-d₆) δ ppm 9.06 (s, 1H), 8.25 (br m, 2H), 8.01 (d, 1H), 7.76 (t, 2H), 7.49 (d, 1H), 7.47 (t, 1H), 7.33 (t, 1H), 7.28 (s, 1H), 7.11 (d, 1H), 7.08 (s, 2H), 7.03 (m, 2H), 6.56 (d, 1H), 6.17 (m, 1H), 5.00 (s, 2H), 4.86 (br m, 1H), 4.64 (d, 2H), 4.02 (s, 2H), 3.88 (m, 6H), 3.79 (br m, 2H), 3.43, 3.35 (m, m, total 14H), 3.22 (s, 3H), 2.80 (m, 2H), 2.57 (m, 2H), 2.09 (s, 3H), 1.37 (br m, 2H), 1.28-0.90 (m, 10H), 0.77-0.82 (m, 6H). MS (ESI) m/e 1407.4 (M−1)⁻.

### 2.11. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon KT)

### 2.11.1. (2S,3R,4S,5S,6S)-2-(4-formyl-3-hydroxyphenoxy)-6-(methoxycarbonyl)tetrahydro-2H-pyran-3,4,5-triyl triacetate

2,4-Dihydroxybenzaldehyde (15 g) and (2S,3R,4S,5S,6S)-2-bromo-6-(methoxycarbonyl)tetrahydro-2H-pyran-3,4,5-triyl triacetate (10 g) were dissolved in acetonitrile followed by the addition of silver carbonate (10 g) and the reaction was heated to 49° C. After stirring for 4 hours, the reaction was cooled, filtered and concentrated. The crude title compound was suspended in dichloromethane and was filtered through diatomaceous earth and concentrated. The residue was purified by silica gel chromatography, eluting with ethyl acetate/heptane, to provide the title compound.

### 2.11.2. (2S,3R,4S,5S,6S)-2-(3-hydroxy-4-(hydroxymethyl)phenoxy)-6-(methoxycarbonyl)tetrahydro-2H-pyran-3,4,5-triyl triacetate

A solution of Example 2.11.1 (16.12 g) in tetrahydrofuran (200 mL) and methanol (200 mL) was cooled to 0° C. and sodium borohydride (1.476 g) was added portionwise. The reaction was stirred for 20 minutes and quenched with a 1:1 mixture of water:aqueous saturated sodium bicarbonate solution (400 mL). The resulting solids were filtered off and rinsed with ethyl acetate. The phases were separated and the aqueous layer extracted four times with ethyl acetate. The combined organic layers were dried over magnesium sulfate, filtered, and concentrated. The crude title compound was purified via silica gel chromatography eluting with heptane/ethyl acetate to provide the title compound. MS (ESI) m/e 473.9 (M+NH₄)⁺.

### 2.11.3. (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate

To Example 2.11.2 (7.66 g) and tert-butyldimethylsilyl chloride (2.78 g) in dichloromethane (168 mL) at −5° C. was added imidazole (2.63 g) and the reaction was stirred overnight allowing the internal temperature of the reaction to warm to 12° C. The reaction mixture was poured into saturated aqueous ammonium chloride and extracted four times with dichloromethane. The combined organics were washed with brine, dried over magnesium sulfate, filtered and concentrated. The crude title compound was purified via silica gel chromatography eluting with heptane/ethyl acetate to provide the title compound. MS (ESI) m/e 593.0 (M+Na)⁺.

### 2.11.4. (2S,3R,4S,5S,6S)-2-(3-(2-(2-((((9H-fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate

To Example 2.11.3 (5.03 g) and triphenylphosphine (4.62 g) in toluene (88 mL) was added di-tert-butyl-azodicarboxylate (4.06 g) and the reaction was stirred for 30 minutes. (9H-Fluoren-9-yl)methyl (2-(2-hydroxyethoxy)ethyl)carbamate was added and the reaction was stirred for an additional 1.5 hours. The reaction was loaded directly onto silica gel and was eluted with heptane/ethyl acetate to provide the title compound.

### 2.11.5. (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate

Example 2.11.4 (4.29 g) was stirred in a 3:1:1 solution of acetic acid:water:tetrahydrofuran (100 mL) overnight. The reaction was poured into saturated aqueous sodium bicarbonate and extracted with ethyl acetate. The organic layer was dried over magnesium sulfate, filtered and concentrated. The crude title compound was purified via silica gel chromatography, eluting with heptane/ethyl acetate, to provide the title compound.

### 2.11.6. (2S,3R,4S,5S,6S)-2-(3-(2-(2-((((9H-fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate

To a solution of Example 2.11.5 (0.595 g) and bis(4-nitrophenyl) carbonate (0.492 g) in N,N-dimethylformamide (4 mL) was added N-ethyl-N-isopropylpropan-2-amine (0.212 mL). After 1.5 hours, the reaction was concentrated under high vacuum. The reaction was loaded directly onto silica gel and eluted using heptane/ethyl acetate to provide the title compound. MS (ESI) m/e 922.9 (M+Na)⁺.

### 2.11.7. 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (150 mg) was dissolved in dimethylformamide (0.5 mL). Example 2.11.6 (190 mg) and N,N-diisopropylethylamine (0.30 mL) were added. The reaction was stirred at room temperature overnight. Then more Example 2.11.6 (70 mg) and more N,N-diisopropylethylamine (0.10 mL) were added and the reaction was allowed to stir for another 24 hours. The reaction was then concentrated and the residue was dissolved in tetrahydrofuran (2 mL) and methanol (2 mL), then 1.94N aqueous lithium hydroxide monohydrate (1.0 mL) was added and the mixture stirred at room temperature for one hour. Purification by reverse phase chromatography (C18 column), eluting with 10-90% acetonitrile in 0.1% TFA/water, provided the title compound as a trifluoroacetic acid salt. MS (ESI) m/e 1261.4 (M−H)⁻.

### 2.11.8. 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-(3-(2,5-dioxo-2,5-dihydro-1H-pyrrol-1-yl)propanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

Example 2.11.7 (19 mg) was dissolved in dimethylformamide (0.3 mL), then 2,5-dioxopyrrolidin-1-yl 3-(2,5-dioxo-2,5-dihydro-1H-pyrrol-1-yl)propanoate (6 mg) and N-ethyl-N-isopropylpropan-2-amine (13 μL) were added. The reaction was stirred for three hours at room temperature, then purification by reverse phase chromatography (C18 column), eluting with 10-90% acetonitrile in 0.1% TFA/water, provided the title compound. ¹H NMR (400 MHz, dimethyl sulfoxide-d₆) δ ppm 12.70 (v br s, 1H), 8.00 (m, 2H), 7.76 (t, 2H), 7.50 (d, 1H), 7.46 (t, 1H), 7.34 (t, 1H), 7.28 (s, 1H), 7.19 (d, 1H), 7.00 (m, 2H), 6.97 (s, 2H), 6.66 (d, 1H), 6.60 (dd, 1H), 5.06 (br m, 1H), 5.00 (s, 2H), 4.96 (s, 2H), 4.09 (m, 2H), 3.88 (m, 6H), 3.80 (br m, 3H), 3.71 (m, 2H), 3.59 (t, 2H), 3.44, 3.38 (both m, total 8H), 3.28 (m, 4H), 3.18 (m, 4H), 2.82 (br m, 2H), 2.33 (t, 2H), 2.09 (s, 3H), 1.33 (br m, 2H), 1.28-0.90 (m, 10H), 0.82 (m, 6H). MS (ESI) m/e 1412.4 (M−H)⁻.

### 2.12. Synthesis of 6-[8-(1,3-benzothiazol-2-ylcarbamoyl)-5-methoxy-3,4-dihydroisoquinolin-2(1H)-yl]-3-(1-{[3-(2-{[({(2E)-3-[4-{[(2S,3R,4S,5S,6S)-6-carboxy-3,4,5-trihydroxytetrahydro-2H-pyran-2-yl]oxy}-3-({3-[({[(2E)-3-(4-{[(2S,3R,4S,5S,6S)-6-carboxy-3,4,5-trihydroxytetrahydro-2H-pyran-2-yl]oxy}-3-[(3-{[3-(2,5-dioxo-2,5-dihydro-1H-pyrrol-1-yl)propanoyl]amino}propanoyl)amino]phenyl)prop-2-en-1-yl]oxy}carbonyl)amino]propanoyl}amino)phenyl]prop-2-en-1-yl}oxy)carbonyl](2-methoxyethyl)amino}ethoxy)-5,7-dimethyltricyclo[3.3.1.1^{3,7}]dec-1-yl]methyl}-5-methyl-1H-pyrazol-4-yl)pyridine-2-carboxylic acid (Synthon KU)

### 2.12.1. 3(1-((3-(2-(((((E)-3-(3-(3-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)amino)propanamido)-4-(((2S,3R,4S,5S,6S)-6-carboxy-3,4,5-trihydroxtetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was isolated as a by-product during the synthesis of Example 2.9.1. MS (ESI) m/e 1708.5 (M−H)⁻.

### 2.12.2. 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-(((((E)-3-(4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)phenyl)allyl)oxy)carbonyl)amino) propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.12.1 for Example 2.11.7 in Example 2.11.8. ¹H NMR (400 MHz, dimethyl sulfoxide-d₆) δ ppm 8.99 (s, 1H), 8.97 (s, 1H), 8.17 (br s, 2H), 8.00 (br t, 1H), 7.94 (d, 1H), 7.70 (dd, 2H), 7.41 (m, 2H), 7.27 (t, 1H), 7.04 (br d, 2H), 6.97 (d, 2H), 6.93 (m, 2H), 6.89 (s, 2H), 6.52 (d, 1H), 6.49 (d, 1H), 6.11 (m, 2H), 4.93 (s, 2H), 4.80 (m, 2H), 4.56 (m, 4H), 3.83 (m, 7H), 3.72 (br d, 2H), 3.53 (m, 2H), 3.45-3.28 (m, 28H), 3.15 (s, 3H), 2.74 (m, 2H), 2.48 (m, 4H), 2.26 (t, 2H), 2.02 (s, 3H), 1.28 (br d, 2H), 1.17 (m, 4H), 1.02 (m, 4H), 0.89 (m, 2H), 0.2 (m, 6H). MS (ESI−) m/e 1859.5 (M−H)⁻.

### 2.13. Synthesis of 4-[({[2-(2-{2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-(beta-D-glucopyranuronosyloxy)phenoxy}ethoxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon KV)

### 2.13.1. 3-(1-((3-(2-((((2-(2-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)amino)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was isolated as a by-product during the synthesis of Example 2.11.7. MS (ESI) m/e 1690.5 (M−H)⁻.

### 2.13.2. 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-((((4-(((2S,3R,4S,5S,6S)-6-carboxy-3,4,5-trihydroxytetrahydro-2H-pyran-2-yl)oxy)-2-(2-(2-(3-(2,5-dioxo-2,5-dihydro-1H-pyrrol-1-yl)propanamido)ethoxy)ethoxy)benzyl)oxy)carbonyl)amino)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.13.1 for Example 2.11.7 in Example 2.11.8. ¹H NMR (400 MHz, dimethyl sulfoxide-d₆) δ ppm 8.00 (m, 2H), 7.76 (t, 2H), 7.50 (d, 1H), 7.46 (m, 1H), 7.34 (m, 1H), 7.28 (s, 1H), 7.19 (m, 3H), 6.99 (m, 2H), 6.97 (s, 2H), 6.66 (m, 2H), 6.60 (m, 2H), 5.07 (m, 2H) 5.00 (s, 2H), 4.96 (s, 2H), 4.93 (s, 2H), 4.09 (m, 4H), 3.90 (m, 7H), 3.80 (br d, 4H), 3.71 (m, 4H), 3.59 (t, 2H), 3.48, 3.44, 3.38 (all m, total 14H), 3.28 (m, 7H), 3.16 (m, 7H), 2.81 (br m, 2H), 2.33 (t, 2H), 2.09 (s, 3H), 1.35 (br d, 2H), 1.28-0.90 (m, 10H), 0.82 (m, 6H). MS (ESI) m/e 1842.5 (M−H)⁻.

### 2.14. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-[2-(2-{[(2,5-dioxo-2,5-dihydro-1H-pyrrol-1-yl)acetyl]amino}ethoxy)ethoxy]phenyl beta-D-glucopyranosiduronic acid (Synthon KW)

The title compound was prepared by substituting 2,5-dioxopyrrolidin-1-yl 2-(2,5-dioxo-2,5-dihydro-1H-pyrrol-1-yl)acetate for 2,5-dioxopyrrolidin-1-yl 3-(2,5-dioxo-2,5-dihydro-1H-pyrrol-1-yl)propanoate in Example 2.11.8. ¹H NMR (400 MHz, dimethyl sulfoxide-d) δ ppm 12.73 (v br s, 1H), 8.21 (br t, 1H), 8.01 (d, 1H), 7.76 (t, 2H), 7.50 (d, 1H), 7.46 (t, 1H), 7.34 (t, 1H), 7.28 (s, 1H), 7.19 (d, 1H), 7.07 (s, 2H), 6.99 (t, 2H), 6.66 (d, 1H), 6.60 (dd, 1H), 5.06 (br m, 1H), 5.00 (s, 2H), 4.96 (s, 2H), 4.09 (m, 2H), 4.02 (s, 2H), 3.88 (m, 6H), 3.80 (br m, 3H), 3.71 (m, 2H), 3.48 (t, 2H), 3.39 (m, 6H), 3.28, 3.21 (both m, 8H), 2.82 (br m, 2H), 2.09 (s, 3H), 1.33 (br m, 2H), 1.28-0.90 (m, 10H), 0.831 (m, 6H). MS (ESI) m/e 1398.4 (M−H)⁻.

### 2.15. Synthesis of 6-[1-(1,3-benzothiazol-2-ylcarbamoyl)-1,2,3,4-tetrahydroquinolin-7-yl]-3-{1-[(3-{[34-(2,5-dioxo-2,5-dihydro-1H-pyrrol-1-yl)-3-methyl-4,32-dioxo-7,10,13,16,19,22,25,28-octaoxa-3,31-diazatetratriacont-1-yl]oxy}-5,7-dimethyltricyclo[3.3.1.1^{3,7}]dec-1-yl)methyl]-5-methyl-1H-pyrazol-4-yl}pyridine-2-carboxylic acid (Synthon DC)

To a mixture of Example 1.1.14 (30 mg) and 2,5-dioxopyrrolidin-1-yl 1-(2,5-dioxo-2,5-dihydro-1H-pyrrol-1-yl)-3-oxo-7,10,13,16,19,22,25,28-octaoxa-4-azahentriacontan-31-oate (MAL-dPEG8-NHS-Ester) (40.8 mg) in N,N-dimethylformamide (3 mL) at 0° C. was added N,N-diisopropylethylamine (48 μL). The mixture was stirred at 0° C. for 20 minutes and at room temperature for 10 minutes. Acetic acid (23 μL) was added and the mixture was purified by reverse phase chromatography (C18 column), eluting with 20-60% acetonitrile in 0.1% TFA/water, to provide the title compound. MS (ESI) m/e 1332.5 (M+H)⁺.

### 2.16. Synthesis of 4-[({[2-({3-[(4-{6-[8-(1,3-benzothiazol-2-ylcarbamoyl)-5-cyano-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon KZ)

### 2.16.1. 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(8-(benzo[d]thiazol-2-ylcarbamoyl)-5-cyano-3,4-dihydroisoquinolin-2(1H)-yl)picolinic acid

The title compound was prepared by substituting Example 1.13.12 for Example 1.12.10 in Example 2.11.7. MS (ESI) m/e 1200 (M+H), 1198 (M−H)⁻.

### 2.16.2. 4-[({[2-({3-[(4-{6-[8-(1,3-benzothiazol-2-ylcarbamoyl)-5-cyano-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

The title compound was prepared by substituting Example 2.16.1 for Example 2.11.7 in Example 2.11.8. ¹H NMR (400 MHz, dimethyl sulfoxide-d₆) δ ppm 13.06 (bs, 2H), 8.04 (d, 1H), 8.01 (t, 1H), 7.92 (d, 1H), 7.78 (dd, 2H), 7.53 (d, 1H), 7.48 (t, 1H), 7.37 (t, 11), 7.29 (s, 1H), 7.19 (d, 1H), 7.06 (t, 1H), 7.03 (d, 1H), 6.98 (s, 1H), 6.65 (d, 1H), 6.59 (dd, 1H), 5.07 (d, 1H), 4.98 (s, 1H), 4.92 (1H), 4.09 (m, 2H), 3.96 (t, 2H), 3.90 (d, 2H), 3.80 (s, 2H), 3.70 (m, 6H), 3.60 (m, 6H), 3.43 (t, 2H), 3.39 (t, 2H), 3.33 (t, 1H), 3.28 (dd, 1H), 3.16 (m, 4H), 3.03 (q, 2H), 2.33 (t, 2H), 2.09 (s, 3H), 1.37 (s, 2H), 1.25 (q, 4H), 1.11 (q, 4H), 1.00 (dd, 2H), 0.83 (s, 6H). MS (ESI) m/e 1351 (M+H)⁺, 1349 (M−H)⁻.

### 2.17. Synthesis of 4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-({N-[3-(2,5-dioxo-2,5-dihydro-1H-pyrrol-1-yl)propanoyl]-beta-alanyl}amino)phenyl beta-D-glucopyranosiduronic acid (Synthon LW)

The title compound was prepared by substituting Example 2.9.1 for Example 2.11.7 in Example 2.11.8. ¹H NMR (400 MHz, dimethyl sulfoxide-d₆) δ ppm 9.03 (s, 1H), 8.25 (br m, 1H), 8.05 (br t, 1H), 8.01 (d, 1H), 7.76 (t, 2H), 7.49 (d, 1H), 7.47 (t, 1H), 7.33 (t, 1H), 7.28 (s, 1H), 7.10 (d, 1H), 7.05 (m, 1H), 7.00 (m, 2H), 6.96 (s, 2H), 6.56 (d, 1H), 6.17 (m, 1H), 5.00 (s, 2H), 4.86 (br m, 1H), 4.64 (d, 2H), 3.88 (m, 6H), 3.79 (br m, 2H), 3.60 (t, 2H), 3.43, 3.35 (m, m, total 14H), 3.22 (s, 3H), 2.80 (m, 2H), 2.53 (m, 2H), 2.33 (t, 2H), 2.09 (s, 3H), 1.37 (br m, 2H), 1.28-0.90 (m, 10H), 0.82, 0.77 (both s, total 6H). MS (ESI−) m/e 1421.5 (M−H)⁻.

### 2.18. Synthesis of N-[(2,5-dioxo-2,5-dihydro-1H-pyrrol-1-yl)acetyl]-3-sulfo-L-alanyl-N-{5-[(1E)-3-(({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon LY)

### 2.18.1. 3-(1-((3-(2-(((((E)-3-(3-(3-((R)-2-amino-3-sulfopropanamido)propanamido)-4-(((2S,3R,4S,5S,6S)-6-carboxy-3,4,5-trihydroxytetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

To a solution of (R)-2-((((9H-fluoren-9-yl)methoxy)carbonyl)amino)-3-sulfopropanoic acid (29 mg) and 2-(3H-[1,2,3]triazolo[4,5-b]pyridin-3-yl)-1,1,3,3-tetramethylisouronium hexafluorophosphate(V) (28 mg) in N,N-dimethylformamide (0.7 mL) was added N,N-diisopropylethylamine (0.013 mL). After stirring for 2 minutes, the reaction was added to a solution of Example 2.9.1 (70 mg) and N-ethyl-N-isopropylpropan-2-amine (0.035 mL) in N,N-dimethylformamide (0.5 mL) at room temperature, and the mixture was stirred for 3 hours. Diethylamine (0.035 mL) was added to the reaction and stirring was continued for an additional 2 hours. The reaction was diluted with water (1 mL), and purified by prep HPLC using a Gilson system eluting with 10-85% acetonitrile in water containing 0.1% v/v trifluoroacetic acid. The desired fractions were combined and freeze-dried to provide the title compound. MS (ESI) m/e 1421.4 (M−H)⁻.

### 2.18.2. 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-((R)-2-(2-(2,5-dioxo-2,5-dihydro-1H-pyrrol-1-yl)acetamido)-3-sulfopropanamido)propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.18.1 for Example 2.9.1 in Example 2.10. ¹H NMR (400 MHz, dimethyl sulfoxide-d₆) δ ppm 9.12 (s, 1H), 8.32 (d, 1H), 8.22 (br m, 1H), 8.01 (d, 1H), 7.97 (br t, 1H), 7.76 (t, 2H), 7.49 (d, 1H), 7.47 (t, 1H), 7.33 (t, 1H), 7.28 (s, 1H), 7.10 (d, 1H), 7.07 (s 2H), 7.05 (m, 1H), 7.00 (m, 2H), 6.56 (d, 1H), 6.17 (m, 1H), 5.00 (s, 2H), 4.86 (br m, 1H), 4.64 (d, 2H), 4.32 (m, 1H), 4.07 (s, 2H), 3.88 (m, 6H), 3.79 (br m, 2H), 3.43, 3.35 (m, m, total 14H), 3.22 (s, 3H), 2.80 (m, 4H), 2.53 (m, 2H), 2.09 (s, 3H), 1.37 (br m, 2H), 1.28-0.90 (m, 10H), 0.82, 0.77 (both s, total 6H). MS (ESI−) m/e 1558.4 (M−H)⁻.

### 2.19. Synthesis of N-[3-(2,5-dioxo-2,5-dihydro-1H-pyrrol-1-yl)propanoyl]-3-sulfo-L-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon LZ)

The title compound was prepared by substituting Example 2.18.1 for Example 2.11.7 in Example 2.11.8. ¹H NMR (400 MHz, dimethyl sulfoxide-d₆) δ ppm 9.12 (s, 1H), 8.22 (br m, 1H), 8.07 (br d, 1H), 8.01 (d, 1H), 7.89 (brt, 1H), 7.76 (t, 2H), 7.49 (d, 1H), 7.47 (t, 1H), 7.33 (t, 1H), 7.28 (s, 1H), 7.10 (d, 1H), 7.05 (m, 1H), 7.00 (m, 2H), 6.96 (s, 2H), 6.56 (d, 1H), 6.17 (m, 1H), 5.00 (s, 2H), 4.86 (br m, 1H), 4.64 (d, 2H), 4.32 (m, 1H), 3.88 (m, 6H), 3.79 (br m, 2H), 3.60 (t, 2H), 3.43, 3.35 (m, m, total 14H), 3.22 (s, 3H), 2.80 (m, 4H), 2.53 (m, 2H), 2.37 (m, 2H), 2.09 (s, 3H), 1.37 (br m, 2H), 1.28-0.90 (m, 10H), 0.82, 0.77 (both s, total 6H). MS (ESI−) m/e 1572.5 (M−H)⁻.

### 2.20. Synthesis of N-[(2,5-dioxo-2,5-dihydro-1H-pyrrol-1-yl)acetyl]-beta-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon MB)

### 2.20.1. 3-(1-((3-(2-(((((E)-3-(3-(3-(3-aminopropanamido)propanamido)-4-(((2S,3R,4S,5S,6S)-6-carboxy-3,4,5-trihydroxytetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting 3-((((9H-fluoren-9-yl)methoxy)carbonyl)amino)propanoic acid for (R)-2-((((9H-fluoren-9-yl)methoxy)carbonyl)amino)-3-sulfopropanoic acid in Example 2.18.1. MS (ESI−) m/e 1341.5 (M−H)⁻.

### 2.20.2. 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-(3-(2-(2,5-dioxo-2,5-dihydro-1H-pyrrol-1-yl)acetamido)propanamido)propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.20.1 for Example 2.9.1 in Example 2.10. ¹H NMR (400 MHz, dimethyl sulfoxide-d₆) δ ppm 9.06 (s, 1H), 8.25 (br m, 1H), 8.14 (br t, 1H), 8.01 (d, 1H), 7.99 (br m, 1H), 7.76 (t, 2H), 7.49 (d, 1H), 7.47 (t, 1H), 7.33 (t, 1H), 7.28 (s, 1H), 7.10 (d, 1H), 7.07 (s, 2H), 7.05 (m, 1H), 7.00 (m, 2H), 6.56 (d, 1H), 6.17 (m, 1H), 5.00 (s, 2H), 4.86 (br m, 1H), 4.64 (d, 2H), 3.99 (s, 2H), 3.88 (m, 6H), 3.79 (br m, 2H), 3.43, 3.35 (m, m, total 14H), 3.25 (m, 2H), 3.22 (s, 3H), 2.80 (m, 2H), 2.55 (m, 2H), 2.23 (t, 2H), 2.09 (s, 3H), 1.37 (br m, 2H), 1.28-0.90 (m, 10H), 0.82, 0.77 (both s, total 6H). MS (ESI−) m/e 1478.5 (M−H)⁻.

### 2.21. Synthesis of N-[3-(2,5-dioxo-2,5-dihydro-1H-pyrrol-1-yl)propanoyl]-beta-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon MC)

The title compound was prepared by substituting Example 2.20.1 for Example 2.11.7 in Example 2.11.8. ¹H NMR (400 MHz, dimethyl sulfoxide-d₆) δ ppm 9.06 (s, 1H), 8.25 (br m, 1H), 8.01 (d, 1H), 7.94 (br m, 2H), 7.76 (t, 2H), 7.49 (d, 1H), 7.47 (t, 1H), 7.33 (t, 1H), 7.28 (s, 1H), 7.10 (d, 1H), 7.05 (m, 1H), 7.00 (m, 2H), 6.97 (s, 2H), 6.56 (d, 1H), 6.17 (m, 1H), 5.00 (s, 2H), 4.86 (br m, 1H), 4.64 (d, 2H), 3.88 (m, 6H), 3.79 (br m, 2H), 3.60 (t, 2H), 3.43, 3.35 (m, m, total 14H), 3.22 (s, 3H), 3.18 (m, 2H), 2.80 (m, 2H), 2.55 (m, 2H), 2.29 (t, 2H), 2.20 (t, 2H), 2.09 (s, 3H), 1.37 (br m, 2H), 1.28-0.90 (m, 10H), 0.82, 0.77 (both s, total 6H). MS (ESI−) m/e 1492.5 (M−H)⁻.

### 2.22. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[(2,5-dioxo-2,5-dihydro-1H-pyrrol-1-yl)acetyl]-3-sulfo-L-alanyl}amino)ethoxy]ethoxy}phenyl beta-D-glucopyranosiduronic acid (Synthon ME)

### 2.22.1. 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.11.7 for Example 2.9.1 in Example 2.18.1. MS (ESI−) m/e 1412.4 (M−H)⁻.

### 2.22.2. 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-((R)-2-(2-(2,5-dioxo-2,5-dihydro-1H-pyrrol-1-yl)acetamido)-3-sulfopropanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.22.1 for Example 2.9.1 in Example 2.10. ¹H NMR (400 MHz, dimethyl sulfoxide-d₆) δ ppm 8.32 (d, 1H), 8.02 (d, 1H), 7.76 (m, 3H), 7.52 (d, 1H), 7.46 (t, 1H), 7.34 (t, 1H), 7.30 (s, 1H), 7.19 (d, 1H), 7.06 (s, 2H), 7.00 (m, 2H), 6.66 (d, 1H), 6.58 (dd, 1H), 5.06 (br m, 1H), 5.00 (s, 2H), 4.96 (s, 2H), 4.31 (m, 1H), 4.09 (m, 2H), 4.08 (s, 2H), 3.88 (m, 6H), 3.80 (br m, 4H), 3.71 (m, 2H), 3.44, 3.38 (both m, total 8H), 3.28 (m, 4H), 3.18 (m, 4H), 2.82 (br m, 3H), 2.72 (m, 1H), 2.09 (s, 3H), 1.33 (br m, 2H), 1.28-0.90 (m, 10H), 0.84, 0.81 (both s, total 6H). MS (ESI−) m/e 1549.5 (M−H)⁻.

### 2.23. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid (Synthon MF)

The title compound was prepared by substituting Example 2.22.1 for Example 2.11.7 in Example 2.11.8. ¹H NMR (400 MHz, dimethyl sulfoxide-d) δ ppm 12.70 (v br s, 1H), 8.06 (d, 1H), 8.02 (d, 1H), 7.76 (m, 3H), 7.52 (d, 1H), 7.46 (t, 1H), 7.34 (t, 1H), 7.30 (s, 1H), 7.19 (d, 1H), 7.00 (m, 2H), 6.95 (s, 2H), 6.66 (d, 1H), 6.58 (dd, 1H), 5.06 (br m, 1H), 5.00 (s, 2H), 4.96 (s, 2H), 4.31 (m, 1H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 4H), 2.82 (br m, 3H), 2.72 (m, 1H), 2.33 (m, 2H), 2.09 (s, 3H), 1.33 (br m, 2H), 1.28-0.90 (m, 10H), 0.84, 0.81 (both s, total 6H). MS (ESI−) m/e 1563.5 (M−H)⁻.

### 2.24. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[(2,5-dioxo-2,5-dihydro-1H-pyrrol-1-yl)acetyl]-beta-alanyl}amino)ethoxy]ethoxy}phenyl beta-D-glucopyranosiduronic acid (Synthon MH)

### 2.24.1. 3-(1-((3-(2-((((2-(2-(2-(3-aminopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting 3-((((9H-fluoren-9-yl)methoxy)carbonyl)amino)propanoic acid for (R)-2-((((9H-fluoren-9-yl)methoxy)carbonyl)amino)-3-sulfopropanoic acid and Example 2.11.7 for Example 2.9.1 in Example 2.18.1. MS (ESI−) m/e 1332.5 (M−H)⁻.

### 2.24.2. 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-(3-(2-(2,5-dioxo-2,5-dihydro-1H-pyrrol-1-yl)acetamido)propanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.24.1 for Example 2.9.1 in Example 2.10. ¹H NMR (400 MHz, dimethyl sulfoxide-d₆) δ ppm 12.70 (v br s, 1H), 8.14 (t, 1H), 8.02 (d, 1H), 7.92 (t, 1H), 7.76 (t, 2H), 7.52 (d, 1H), 7.46 (t, 1H), 7.34 (t, 1H), 7.28 (s, 1H), 7.19 (d, 1H), 7.06 (s, 2H), 7.00 (m, 2H), 6.66 (d, 1H), 6.58 (dd, 1H), 5.06 (br m, 1H), 5.00 (s, 2H), 4.96 (s, 2H), 4.09 (m, 2H), 3.98 (s, 2H), 3.88 (m, 6H), 3.80 (br m, 4H), 3.71 (m, 2H), 3.44, 3.38 (both m, total 8H), 3.28 (m, 4H), 3.18 (m, 6H), 2.82 (br m, 2H), 2.24 (t, 2H), 2.09 (s, 3H), 1.33 (br m, 2H), 1.28-0.90 (m, 10H), 0.84, 0.81 (both s, total 6H). MS (ESI−) m/e 1469.5 (M−H)⁻.

### 2.25. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[3-(2,5-dioxo-2,5-dihydro-1H-pyrrol-1-yl)propanoyl]-beta-alanyl}amino)ethoxy]ethoxy}phenyl beta-D-glucopyranosiduronic acid (Synthon MI)

The title compound was prepared by substituting Example 2.24.1 for Example 2.11.7 in Example 2.11.8. ¹H NMR (400 MHz, dimethyl sulfoxide-d₆) δ ppm 12.70 (v br s, 1H), 8.02 (d, 1H), 7.94 (t, 1H), 7.88 (t, 1H), 7.76 (t, 2H), 7.52 (d, 1H), 7.46 (t, 1H), 7.34 (t, 1H), 7.28 (s, 1H), 7.19 (d, 1H), 7.00 (m, 2H), 6.95 (s, 2H), 6.66 (d, 1H), 6.58 (dd, 1H), 5.06 (br m, 1H), 5.00 (s, 2H), 4.96 (s, 2H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 6H), 2.82 (br m, 2H), 2.30 (t, 2H), 2.20 (t, 2H), 2.09 (s, 3H), 1.33 (br m, 2H), 1.28-0.90 (m, 10H), 0.84, 0.81 (both s, total 6H). MS (ESI−) m/e 1483.5 (M−H)⁻.

### 2.26. Synthesis of 2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid (Synthon NJ)

### 2.26.1. 4-(2-(2-bromoethoxy)ethoxy)-2-hydroxybenzaldehyde

A solution of 2,4-dihydroxybenzaldehyde (1.0 g), 1-bromo-2-(2-bromoethoxy)ethane (3.4 g) and potassium carbonate (1.0 g) were stirred together in acetonitrile (30 mL) and heated to 75° C. After stirring for 2 days, the reaction was cooled, diluted with ethyl acetate (100 mL), washed with water (50 mL) and brine (50 mL), dried over magnesium sulfate, filtered and concentrated. Purification via silica gel chromatography, eluting using a gradient of 5-30% ethyl acetate/heptane, provided the title compound. MS (ELSD) m/e 290.4 (M+H)⁺.

### 2.26.2. 4-(2-(2-azidoethoxy)ethoxy)-2-hydroxybenzaldehyde

To a solution of Example 2.26.1 (1.26 g) in N,N-dimethylformamide (10 mL) was added sodium azide (0.43 g) and the reaction was stirred at room temperature overnight. The reaction was diluted with diethyl ether (100 mL), washed with water (50 mL) and brine (50 mL), dried over magnesium sulfate, filtered, and concentrated. Purification via silica gel chromatography, eluting with a gradient of 5-30% ethyl acetate/heptane, gave the title compound. MS (ELSD) m/e 251.4 (M+H)⁺.

### 2.26.3. (2S,3R,4S,5S,6S)-2-(5-(2-(2-azidoethoxy)ethoxy)-2-formylphenoxy)-6-(methoxycarbonyl)tetrahydro-2H-pyran-3,4,5-triyl triacetate

A solution of Example 2.26.2 (0.84 g), (3R,4S,5S,6S)-2-bromo-6-(methoxycarbonyl)tetrahydro-2H-pyran-3,4,5-triyl triacetate (1.99 g) and silver (I) oxide (1.16 g) were stirred together in acetonitrile (15 mL). After stirring overnight, the reaction was diluted with dichloromethane (20 mL), diatomaceous earth was added and the reaction filtered and concentrated. Purification via silica gel chromatography, eluting with a gradient of 5-75% ethyl acetate/heptane, gave the title compound.

### 2.26.4. (2S,3R,4S,5S,6S)-2-(5-(2-(2-azidoethoxy)ethoxy)-2-(hydroxymethyl)phenoxy)-6-(methoxycarbonyl)tetrahydro-2H-pyran-3,4,5-triyl triacetate

A solution of Example 2.26.3 (0.695 g) in methanol (5 mL) and tetrahydrofuran (2 mL) was cooled to 0° C. Sodium borohydride (0.023 g) was added, and the reaction was warmed to room temperature. After stirring for a total of 1 hour, the reaction was poured into a mixture of ethyl acetate (75 mL) and water (25 mL) and saturated aqueous sodium bicarbonate (10 mL) was added. The organic layer was separated, washed with brine (50 mL), dried over magnesium sulfate, filtered, and concentrated. Purification via silica gel chromatography, eluting with a gradient of 5-85% ethyl acetate/heptane, gave the title compound. MS (ELSD) m/e 551.8 (M−H₂O)⁻.

### 2.26.5. (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate

To Example 2.26.4 (0.465 g) in tetrahydrofuran (20 mL) was added 5% Pd/C (0.1 g) in a 50 mL pressure bottle and the mixture shaken for 16 hours at 30 psi hydrogen. The reaction was then filtered and concentrated to give the title compound which was used without further purification. MS (ELSD) m/e 544.1 (M+H)⁺.

### 2.26.6. (2S,3R,4S,5S,6S)-2-(5-(2-(2-((((9H-fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate

A solution of Example 2.26.5 (0.443 g) in dichloromethane (8 mL) was cooled to 0° C., then N,N-diisopropylethylamine (0.214 mL) and (9H-fluoren-9-yl)methyl carbonochloridate (0.190 g) were added. After 1 hour, the reaction was concentrated and purified via column chromatography, eluting with 5-95% ethyl acetate/heptane, to give the title compound. MS (ELSD) m/e 748.15 (M−OH)⁻.

### 2.26.7. (2S,3R,4S,5S,6S)-2-(5-(2-(2-((((9H-fluoren-9-yl)methoxy)carbonyl)amino)ethoxy)ethoxy)-2-((((4-nitrophenoxy)carbonyl)oxy)methyl)phenoxy)-6-(methoxycarbonyl)tetrahydro-2H-pyran-3,4,5-triyl triacetate

To a solution of Example 2.26.6 (0.444 g) in N,N-dimethylformamide (5 mL) was added N,N-diisopropylethylamine (0.152 mL) and bis(4-nitrophenyl) carbonate (0.353 g) and the reaction was stirred at room temperature. After 5 hours, the reaction was concentrated and the residue was purified via column chromatography, eluting with 5-90% ethyl acetate/heptane, to give the title compound.

### 2.26.8. 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (360 mg) was dissolved in dimethylformamide (2.5 mL). Example 2.26.7 (450 mg) and N,N-diisopropylethylamine (0.35 mL) were added. The reaction was stirred at room temperature overnight. The reaction was then concentrated and the residue dissolved in tetrahydrofuran (2.5 mL) and methanol (2.5 mL). Aqueous lithium hydroxide monohydrate (1.94N, 2.2 mL) was added, and the mixture was stirred at room temperature for one hour. Purification by reverse phase chromatography (C18 column), eluting with 10-90% acetonitrile in 0.1% TFA/water, provided the title compound as a trifluoroacetic acid salt. MS (ESI) m/e 1261.4 (M−H)⁻.

### 2.26.9. 3-(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.26.8 for Example 2.9.1 in Example 2.18.1. MS (ESI−) m/e 1412.4 (M−H)⁻.

### 2.26.10.6-(8-(benzo[d]thiazol-2-ylcarbamoyl)-5-methoxy-3,4-dihydroisoquinolin-2(1H)-yl)-3-(1-((3-(2-((((2-(((2S,3R,4S,5S,6S)-6-carboxy-3,4,5-trihydroxytetrahydro-2H-pyran-2-yl)oxy)-4-(2-(2-((R)-2-(3-(2,5-dioxo-2,5-dihydro-1H-pyrrol-1-yl)propanamido)-3-sulfopropanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.26.9 for Example 2.11.7 in Example 2.11.8. ¹H NMR (400 MHz, dimethyl sulfoxide-d₆) δ ppm 12.70 (v br s, 1H), 8.06 (d, 1H), 8.02 (d, 1H), 7.76 (t, 3H), 7.52 (d, 1H), 7.46 (t, 1H), 7.34 (t, 1H), 7.30 (s, 1H), 7.19 (d, 1H), 7.00 (m, 2H), 6.95 (s, 2H), 6.70 (d, 1H), 6.58 (dd, 1H), 5.06 (br m, 1H), 5.00 (s, 2H), 4.96 (s, 2H), 4.31 (m, 1H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 4H), 2.82 (br m, 3H), 2.72 (m, 1H), 2.33 (m, 2H), 2.09 (s, 3H), 1.33 (br m, 2H), 1.28-0.90 (m, 10H), 0.84, 0.81 (both s, total 6H). MS (ESI−) m/e 1563.5 (M−H)⁻.

### 2.27. Synthesis of 2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid (Synthon NK)

The title compound was prepared by substituting Example 2.26.9 for Example 2.9.1 in Example 2.9.2. ¹H NMR (400 MHz, dimethyl sulfoxide-d₆) δ ppm 12.70 (v br s, 1H), 8.06 (d, 1H), 8.02 (d, 1H), 7.76 (t, 3H), 7.52 (d, 1H), 7.46 (t, 1H), 7.34 (t, 1H), 7.30 (s, 1H), 7.19 (d, 1H), 7.00 (m, 2H), 6.95 (s, 2H), 6.70 (d, 1H), 6.58 (dd, 1H), 5.06 (br m, 1H), 5.00 (s, 2H), 4.96 (s, 2H), 4.31 (m, 1H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 4H), 2.82 (br m, 3H), 2.72 (m, 1H), 2.33 (m, 2H), 2.09 (s, 3H), 1.46 (br m, 4H) 1.33 (br m, 2H), 1.28-0.90 (m, 12H), 0.84, 0.81 (both s, total 6H). MS (ESI−) m/e 1605.4 (M−H)⁻.

### 2.28. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid (Synthon NL)

### 2.28.1. (2S,3R,4S,5S,6S)-2-(3-(3-((((9H-fluoren-9-yl)methoxy)carbonyl)amino)propoxy)-4-formylphenoxy)-6-(methoxycarbonyl)tetrahydro-2H-pyran-3,4,5-triyl triacetate

To a solution of(9H-fluoren-9-yl)methyl (3-hydroxypropyl)carbamate (0.245 g) and triphenylphosphine (0.216 g) in tetrahydrofuran (2 mL) at 0° C. was added diisopropyl azodicarboxylate (0.160 mL) dropwise. After stirring for 15 minutes, Example 2.11.1 (0.250 g) was added, the ice bath was removed, and the reaction was allowed to warm to room temperature. After 2 hours, the reaction was concentrated, loaded onto silica gel, and eluted using a gradient of 5-70% ethyl acetate/hexanes to give the title compound. MS (APCI) m/e 512.0 (M−FMOC)⁻.

### 2.28.2. (2S,3R,4S,5S,6S)-2-(3-(3-((((9H-fluoren-9-yl)methoxy)carbonyl)amino)propoxy)-4-(hydroxymethyl)phenoxy)-6-(methoxycarbonyl)tetrahydro-2H-pyran-3,4,5-triyl triacetate

To a suspension of Example 2.28.1 (0.233 g) in methanol (3 mL) and tetrahydrofuran (1 mL) was added sodium borohydride (6 mg). After 30 minutes, the reaction was poured into ethyl acetate (50 mL) and water (25 mL), followed by the addition of sodium bicarbonate (5 mL). The organic layer was separated, washed with brine (25 mL), dried over magnesium sulfate, filtered, and concentrated. Silica gel chromatography, eluting with a gradient of 5-80% ethyl acetate/heptane, gave the title compound. MS (APCI) m/e 718.1 (M−0H)⁻.

### 2.28.3. (2S,3R,4S,5S,6S)-2-(3-(3-((((9H-fluoren-9-yl)methoxy)carbonyl)amino)propoxy)-4-((((4-nitrophenoxy)carbonyl)oxy)methyl)phenoxy)-6-(methoxycarbonyl)tetrahydro-2H-pyran-3,4,5-triyl triacetate

To a solution of Example 2.28.2 (0.140 g) and bis(4-nitrophenyl) carbonate (0.116 g) in N,N-dimethylformamide (1 mL) was added N-ethyl-N-isopropylpropan-2-amine (0.050 mL). After 1.5 hours, the reaction was concentrated under high vacuum, loaded onto silica gel, and eluted using a gradient of 10-70% ethyl acetate/heptane to give the title compound.

### 2.28.4. 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.28.3 for Example 2.26.7 in Example 2.26.8. MS (ESI−) m/e 1231.3 (M−H)⁻.

### 2.28.5. 3-(1-((3-(2-((((2-(3-((R)-2-amino-3-sulfopropanamido)propoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.28.4 for Example 2.9.1 in Example 2.18.1. MS (ESI−) m/e 1382.4 (M−H)⁻.

### 2.28.6. 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(3-((R)-2-(6-(2,5-dioxo-2,5-dihydro-1H-pyrrol-1-yl)hexanamido)-3-sulfopropanamido)propoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.28.5 for Example 2.9.1 in Example 2.9.2. ¹H NMR (400 MHz, dimethyl sulfoxide-d₆) δ ppm 8.01 (d, 1H), 7.85 (m, 2H), 7.76 (m, 2H), 7.52 (d, 1H), 7.46 (t, 1H), 7.34 (m, 1H), 7.30 (s, 1H), 7.16 (d, 1H), 7.00 (m, 3H), 6.97 (s, 2H), 6.64 (d, 1H), 6.56 (dd, 1H), 5.04 (br m, 1H), 5.00 (s, 2H), 4.96 (s, 2H), 4.28 (m, 1H), 3.97 (m. 2H), 3.88 (m, 6H), 3.80 (m, 2H), 3.71 (m, 2H), 3.37 (m, 8H), 3.27 (m, 4H), 3.17 (m, 4H), 2.90-2.65 (m, 4H), 2.09 (s, 3H), 2.05 (t, 2H), 1.81 (m, 2H), 1.46 (br m, 4H), 1.33 (br m, 2H), 1.28-0.90 (m, 12H), 0.84, 0.81 (both s, total 6H). MS (ESI−) m/e 1575.5 (M−H)⁻.

### 2.29. Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid (Synthon NM)

### 2.29.1. 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.28.3 for Example 2.26.7 and Example 1.9.11 for Example 1.12.10 in Example 2.26.8. MS (ESI−) m/e 1187.4 (M−H)⁻.

### 2.29.2. 3-(1-((3-(2-((((2-(3-((R)-2-amino-3-sulfopropanamido)propoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.29.1 for Example 2.9.1 in Example 2.18.1. MS (ESI−) m/e 1338.3 (M−H)⁻.

### 2.29.3. 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(3-((R)-2-(6-(2,5-dioxo-2,5-dihydro-1H-pyrrol-1-yl)hexanamido)-3-sulfopropanamido)propoxy)benzyl)oxy)carbonyl)(methyl)amino) ethoxy)-5,7-dimethyladamantan-1-yl)methyl)-5-methyl-1H-pyrazol-4-yl)picolinic acid

The title compound was prepared by substituting Example 2.29.2 for Example 2.9.1 in Example 2.9.2. ¹H NMR (400 MHz, dimethyl sulfoxide-d₆) δ ppm 8.01 (d, 1H), 7.85 (m, 2H), 7.76 (m, 2H), 7.52 (d, 1H), 7.46 (t, 1H), 7.34 (m, 1H), 7.30 (s, 1H), 7.16 (d, 1H), 7.00 (m, 3H), 6.97 (s, 2H), 6.64 (d, 1H), 6.56 (dd, 1H), 5.04 (br m, 1H), 5.00 (s, 2H), 4.96 (s, 2H), 4.28 (m, 1H), 3.97 (m, 2H), 3.88 (m, 6H), 3.80 (m, 2H), 3.44 (m, 6H), 3.28 (m, 4H), 3.17 (m, 2H), 2.90-2.65 (m, 4H), 2.09 (s, 3H), 2.05 (t, 2H), 1.81 (m, 2H), 1.46 (br m, 4H), 1.33 (br m, 2H), 1.28-0.90 (m, 12H), 0.84, 0.81 (both s, total 6H). MS (ESI−) m/e 1531.5 (M−H)⁻.

### 2.30. Synthesis of N-[6-(2,5-dioxo-2,5-dihydro-1H-pyrrol-1-yl)hexanoyl]-L-valyl-N-{4-[({[(3S)-1-{8-(1,3-benzothiazol-2-ylcarbamoyl)-2-[6-carboxy-5-(1-{[3-(2-methoxyethoxy)-5,7-dimethyltricyclo[3.3.1.1^{3,7}]dec-1-yl]methyl}-5-methyl-1H-pyrazol-4-yl)pyridin-2-yl]-1,2,3,4-tetrahydroisoquinolin-6-yl}pyrrolidin-3-yl]carbamoyl}oxy)methyl]phenyl}-L-alaninamide (Synthon NR)

Example 1.17.10 (40 mg) was dissolved in dimethyl sulfoxide (0.3 mL), and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (31 mg) and triethylamine (33 μL) were added. The reaction mixture was stirred for 72 hours at room temperature, and purification by reverse phase chromatography (C18 column), eluting with 10-90% acetonitrile in 0.1% TFA water, provided the title compound. MS (ESI) m/e 1357.4 (M+H)⁺, 1355.5 (M−H)⁻.

### 2.31. Synthesis of N-[6-(2,5-dioxo-2,5-dihydro-1H-pyrrol-1-yl)hexanoyl]-L-valyl-N-{4-[({[2-((3-[(4-{6-[8-(1,3-benzothiazol-2-ylcarbamoyl)-3,4-dihydroisoquinolin-2(1H)-yl]-2-carboxypyridin-3-yl-5-methyl-1H-pyrazol-1-yl)methyl]-5,7-dimethyltricyclo[3.3.1.1^{3,7}]dec-1-yl}oxy)ethyl](2-sulfamoylethyl)carbamoyl}oxy)methyl]phenyl}-N⁵-carbamoyl-L-ornithinamide (Synthon EB)

The title compound was prepared as described in previous examples. ¹H NMR (500 MHz, dimethyl sulfoxide-d) δ ppm 12.85 (s, 1H), 9.98 (s, 1H), 8.00-8.09 (m, 2H), 7.78 (t, 2H), 7.61 (t, 3H), 7.40-7.53 (m, 3H), 7.33-7.39 (m, 2H), 7.25-7.30 (m, 3H), 6.86-7.00 (m, 5H), 5.99 (s, 1H), 4.86-5.10 (m, 4H), 4.38 (s, 1H), 4.10-4.26 (m, 1H), 3.88 (t, 2H), 3.80 (d, 2H), 3.33-3.39 (m, 2H), 3.30 (d, 2H), 3.18-3.26 (m, 2H), 2.88-3.06 (m, 5H), 2.04-2.24 (m, 5H), 1.87-2.00 (m, 1H), 1.28-1.74 (m, 10H), 0.89-1.27 (m, 12H), 0.74-0.87 (m, 12H). MS (ESI) m/e 1451.3 (M+H)⁺.

### 2.32. Synthesis of Control Synthon 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyl-5,7-dimethyltricyclo[3.3.1.1^{3,7}]dec-1-yl}oxy)ethyl](methyl)carbamoyl}oxy)methyl]-2-({N-[6-(2,5-dioxo-2,5-dihydro-1H-pyrrol-1-yl)hexanoyl]-beta-alanyl}amino)phenyl beta-D-glucopyranosiduronic acid (Synthon H)

### 2.32.1. (2S,3R,4S,5S,6S)-2-(4-formyl-2-nitrophenoxy)-6-(methoxycarbonyl)tetrahydro-2H-pyran-3,4,5-triyl triacetate

To a solution of (2R,3R,4S,5S,6S)-2-bromo-6-(methoxycarbonyl)tetrahydro-2H-pyran-3,4,5-triyl triacetate (4 g) in acetonitrile (100 mL)) was added silver(I) oxide (10.04 g) and 4-hydroxy-3-nitrobenzaldehyde (1.683 g). The reaction mixture was stirred for 4 hours at room temperature and filtered. The filtrate was concentrated, and the residue was purified by silica gel chromatography, eluting with 5-50% ethyl acetate in heptanes, to provide the title compound. MS (ESI) m/e (M+18)⁺.

### 2.32.2. (2S,3R,4S,5S,6S)-2-(4-(hydroxymethyl)-2-nitrophenoxy)-6-(methoxycarbonyl)tetrahydro-2H-pyran-3,4,5-triyl triacetate

To a solution of Example 2.32.1 (6 g) in a mixture of chloroform (75 mL) and isopropanol (18.75 mL) was added 0.87 g of silica gel. The resulting mixture was cooled to 0° C., NaBH₄ (0.470 g) was added, and the resulting suspension was stirred at 0° C. for 45 minutes. The reaction mixture was diluted with dichloromethane (100 mL) and filtered through diatomaceous earth. The filtrate was washed with water and brine and concentrated to give the crude product, which was used without further purification. MS (ESI) m/e (M+NH₄)⁺:

### 2.323. (2S,3R,4S,5S,6S)-2-(2-amino-4-(hydroxymethyl)phenoxy)-6-(methoxycarbonyl)tetrahydro-2H-pyran-3,4,5-triyl triacetate

A stirred solution of Example 2.32.2 (7 g) in ethyl acetate (81 mL) was hydrogenated at 20° C. under 1 atmosphere H₂, using 10% Pd/C (1.535 g) as a catalyst for 12 hours. The reaction mixture was filtered through diatomaceous earth, and the solvent was evaporated under reduced pressure. The residue was purified by silica gel chromatography, eluting with 95/5 dichloromethane/methanol, to give the title compound.

### 2.32.4. 3-((((9H-fluoren-9-yl)methoxy)carbonyl)amino)propanoic acid

3-Aminopropanoic acid (4.99 g) was dissolved in 10, aqueous Na₂CO₃ solution (120 mL) in a 500 mL flask and cooled with an ice bath. To the resulting solution, (9H-fluoren-9-yl)methyl carbonochloridate (14.5 g) in 1,4-dioxane (100 mL) was gradually added. The reaction mixture was stirred at room temperature for 4 hours, and water (800 mL) was then added. The aqueous phase layer was separated from the reaction mixture and washed with diethyl ether (3×750 mL). The aqueous layer was acidified with 2N HCl aqueous solution to a pH value of 2 and extracted with ethyl acetate (3×750 mL). The organic layers were combined and concentrated to obtain crude product. The crude product was recrystallized in a mixed solvent of ethyl acetate:hexane 1:2 (300 mL) to give the title compound.

### 2.32.5. (9H-fluoren-9-yl)methyl (3-chloro-3-oxopropyl)carbamate

To a solution of Example 2.32.4 in dichloromethane (160 mL) was added sulfurous dichloride (50 mL). The mixture was stirred at 60° C. for 1 hour. The mixture was cooled and concentrated to give the title compound.

### 2.32.6. (2S,3R,4S,5S,6S)-2-(2-(3-((((9H-fluoren-9-yl)methoxy)carbonyl)amino)propanamido)-4-(hydroxymethyl)phenoxy)-6-(methoxycarbonyl)tetrahydro-2H-pyran-3,4,5-triyl triacetate

To a solution of Example 2.32.3 (6 g) in dichloromethane (480 mL) was added N,N-diisopropylethylamine (4.60 mL). Example 2.32.5 (5.34 g) was added, and the mixture was stirred at room temperature for 30 minutes. The mixture was poured into saturated aqueous sodium bicarbonate and was extracted with ethyl acetate. The combined extracts were washed with water and brine and were dried over sodium sulfate. Filtration and concentration gave a residue that was purified via radial chromatography, using 0-100% ethyl acetate in petroleum ether as mobile phase, to give the title compound.

### 2.32.7. (2S,3R,4S,5S,6S)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl triacetate

To a mixture of Example 2.32.6 (5.1 g) in N,N-dimethylformamide (200 mL) was added bis(4-nitrophenyl) carbonate (4.14 g) and N,N-diisopropylethylamine (1.784 mL). The mixture was stirred for 16 hours at room temperature and concentrated under reduced pressure. The crude material was dissolved in dichloromethane and aspirated directly onto a 1 mm radial Chromatotron plate and eluted with 50-100% ethyl acetate in hexanes to give the title compound. MS (ESI) m/e (M+H)⁺.

### 2.32.8. 3-bromo-5,7-dimethyladamantanecarboxylic acid

In a 50 mL round-bottomed flask at 0° C. was added bromine (16 mL). Iron powder (7 g) was then added, and the reaction was stirred at 0° C. for 30 minutes. 3,5-Dimethyladamantane-1-carboxylic acid (12 g) was then added. The mixture was warmed up to room temperature and stirred for 3 days. A mixture of ice and concentrated HCl was poured into the reaction mixture. The resulting suspension was treated twice with Na₂SO₃(50 g in 200 mL water) to destroy bromine and was extracted three times with dichloromethane. The combined organics were washed with 1N aqueous HCl, dried over Na₂SO₄, filtered, and concentrated to give the crude title compound.

### 2.32.9. 3-bromo-5,7-dimethyladamantanemethanol

To a solution of Example 2.32.8 (15.4 g) in tetrahydrofuran (200 mL) was added BH₃ (1M in tetrahydrofuran. 150 mL). The mixture was stirred at room temperature overnight. The reaction mixture was then carefully quenched by adding methanol dropwise. The mixture was then concentrated under vacuum, and the residue was balanced between ethyl acetate (500 mL) and 2N aqueous HCl (100 mL). The aqueous layer was further extracted twice with ethyl acetate, and the combined organic extracts were washed with water and brine, dried over Na₂SO₄, and filtered. Evaporation of the solvent gave the title compound.

### 2.32.10. 1-((3-bromo-5,7-dimethyltricyclo[3.3.1.1^{3,7}]dec-1-yl)methyl)-1H-pyrazole

To a solution of Example 2.32.9 (8.0 g) in toluene (60 mL) was added 1H-pyrazole (1.55 g) and cyanomethylenetributylphosphorane (2.0 g). The mixture was stirred at 90° C. overnight. The reaction mixture was then concentrated and the residue was purified by silica gel column chromatography (10:1 heptane:ethyl acetate) to give the title compound. MS (ESI) m/e 324.2 (M+H)⁺.

### 2.32.11. 2-{[3,5-dimethyl-7-(1H-pyrazol-1-ylmethyl)tricyclo[3.3.1.1^{3,7}]dec-1-yl]oxy}ethanol

To a solution of Example 2.32.10 (4.0 g) in ethane-1,2-diol (12 mL) was added triethylamine (3 mL). The mixture was stirred at 150° C. under microwave conditions (Biotage Initiator) for 45 minutes. The mixture was poured into water (100 mL) and extracted three times with ethyl acetate. The combined organic extracts were washed with water and brine, dried over Na₂SO₄, and filtered. Evaporation of the solvent gave the crude product, which was purified by silica gel chromatography, eluting with 20% ethyl acetate in heptane, followed by 5% methanol in dichloromethane, to give the title compound. MS (ESI) m/e 305.2 (M+H)⁺.

### 2.32.12. 2-({3,5-dimethyl-7-[(5-methyl-1H-pyrazol-1-yl)methyl]tricyclo[3.3.1.1^{3,7}]dec-1-yl}oxy)ethanol

To a cooled (−78° C.) solution of Example 2.32.11 (6.05 g) in tetrahydrofuran (100 mL) was added n-BuLi (40 mL, 2.5M in hexane). The mixture was stirred at −78° C. for 1.5 hours. Iodomethane (10 mL) was added through a syringe, and the mixture was stirred at −78° C. for 3 hours. The reaction mixture was then quenched with aqueous NH₄Cl and extracted twice with ethyl acetate, and the combined organic extracts were washed with water and brine. After drying over Na₂SO₄, the solution was filtered and concentrated, and the residue was purified by silica gel column chromatography, eluting with 5% methanol in dichloromethane, to give the title compound. MS (ESI) m/e 319.5 (M+H)⁺.

### 2.32.13. 1-({3,5-dimethyl-7-[2-(hydroxy)ethoxy]tricyclo[3.3.1.1^{3,7}]dec-1-yl}methyl)-4-iodo-5-methyl-1H-pyrazole

To a solution of Example 2.32.12 (3.5 g) in N,N-dimethylformamide (30 mL) was added N-iodosuccinimide (3.2 g). The mixture was stirred at room temperature for 1.5 hours. The reaction mixture was then diluted with ethyl acetate (600 mL) and washed with aqueous NaHSO₃, water, and brine. After drying over Na₂SO₄, the solution was filtered and concentrated and the residue was purified by silica gel chromatography (20% ethyl acetate in dichloromethane) to give the title compound. MS (ESI) m/e 445.3 (M+H)⁺.

### 2.32.14. 2-({3-[(4-iodo-5-methyl-1H-pyrazol-1-yl)methyl]-5,7-dimethyltricyclo[3.3.1.1^{3,7}]dec-1-yl}oxy)ethyl methanesulfonate

To a cooled solution of Example 2.32.13 (6.16 g) in dichloromethane (100 mL) was added triethylamine (4.21 g) followed by methanesulfonyl chloride (1.6 g). The mixture was stirred at room temperature for 1.5 hours. The reaction mixture was then diluted with ethyl acetate (600 mL) and washed with water and brine. After drying over Na₂SO₄, the solution was filtered and concentrated, and the residue was used in the next reaction without further purification. MS (ESI) m/e 523.4 (M+H)⁺.

### 2.32.15. 1-({3,5-dimethyl-7-[2-(methylamino)ethoxy]tricyclo[3.3.1.1^{3,7}]dec-1-yl}methyl)-4-iodo-5-methyl-1H-pyrazole

A solution of Example 2.32.14 (2.5 g) in 2M methylamine in methanol (15 mL) was stirred at 100° C. for 20 minutes under microwave conditions (Biotage Initiator). The reaction mixture was concentrated under vacuum. The residue was then diluted with ethyl acetate (400 mL) and washed with aqueous NaHCO₃, water and brine. After drying over Na₂SO₄, the solution was filtered and concentrated, and the residue was used in the next reaction without further purification. MS (ESI) m/e 458.4 (M+H)⁺.

### 2.32.16. tert-butyl [2-({3-[(4-iodo-5-methyl-1H-pyrazol-1-yl)methyl]-5,7-dimethyltricyclo[3.3.1.1^{3,7}]dec-1-yl}oxy)ethyl]methylcarbamate

To a solution of Example 2.32.15 (2.2 g) in tetrahydrofuran (30 mL) was added di-tert-butyl dicarbonate (1.26 g) and a catalytic amount of 4-dimethylaminopyridine. The mixture was stirred at room temperature for 1.5 hours and diluted with ethyl acetate (300 mL). The solution was washed with saturated aqueous NaHCO₃, water (60 mL), and brine (60 mL). The organic layer was dried with Na₂SO₄, filtered, and concentrated. The residue was purified by silica gel chromatography, eluting with 20% ethyl acetate in dichloromethane, to give the title compound. MS (ESI) m/e 558.5 (M+H)⁺.

### 2.32.17. 6-fluoro-3-bromopicolinic acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour, and the resulting mixture was stirred for another 30 minutes, then warmed to 35° C. and stirred overnight. The reaction was cooled to room temperature, and then adjusted to pH 4 with aqueous NaH₂PO₄ solution. The resulting solution was extracted three times with dichloromethane, and the combined extracts were washed with brine, dried over sodium sulfate, filtered and concentrated to provide the title compound.

### 2.32.18. Tert-butyl 3-bromo-6-fluoropicolinate

Para-toluenesulfonyl chloride (27.6 g) was added to a solution of Example 2.32.17 (14.5 g) and pyridine (26.7 mL) in dichloromethane (100 mL) and tert-butanol (80 mL) at 0° C. The reaction was stirred for 15 minutes, warmed to room temperature, and stirred overnight. The solution was concentrated and partitioned between ethyl acetate and aqueous Na₂CO₃ solution. The layers were separated, and the aqueous layer extracted with ethyl acetate. The organic layers were combined, rinsed with aqueous Na₂CO₃ solution and brine, dried over sodium sulfate, filtered, and concentrated to provide the title compound.

### 2.32.19. methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 2.32.18 (15 g) in dimethyl sulfoxide (100 mL) was added N,N-diisopropylethylamine (12 mL). The mixture was stirred at 50° C. for 24 hours. The mixture was then diluted with ethyl acetate (500 mL), washed with water and brine, and dried over Na₂SO₄. Filtration and evaporation of the solvent gave a residue that was purified by silica gel chromatography, eluting with 20% ethyl acetate in heptane, to give the title compound. MS (ESI) m/e 448.4 (M+H)⁺.

### 2.32.20. methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 2.32.19 (2.25 g) and [1,1′-bis(diphenylphosphino)ferrocene]dichloropalladium(II) (205 mg) in acetonitrile (30 mL) was added triethylamine (3 mL) and pinacolborane (2 mL). The mixture was stirred at reflux for 3 hours. The mixture was diluted with ethyl acetate (200 mL) and washed with water and brine, and dried over Na₂SO₄. Filtration, evaporation of the solvent, and silica gel chromatography (eluted with 20% ethyl acetate in heptane) gave the title compound. MS (ESI) m/e 495.4 (M+H)⁺.

### 2.32.21. methyl 2-(6-(tert-butoxycarbonyl)-5-(1-((3-(2-((tert-butoxycarbonyl)methyl)amino)ethoxy)-5,7-dimethyltricyclo[3.3.1.1^{3,7}]dec-1-yl)methyl)-5-methyl-1H-pyrazol-4-yl)pyridin-2-yl)-1,2,3,4-tetrahydroisoquinoline-8-carboxylate

To a solution of Example 2.32.20 (4.94 g) in tetrahydrofuran (60 mL) and water (20 mL) was added Example 2.32.16 (5.57 g), 1,3,5,7-tetramethyl-8-tetradecyl-2,4,6-trioxa-8-phosphaadamantane (412 mg), tris(dibenzylideneacetone)dipalladium(0) (457 mg), and K₃PO₄ (11 g). The mixture was stirred at reflux for 24 hours. The reaction mixture was cooled, diluted with ethyl acetate (500 mL), washed with water and brine, and dried over Na₂SO₄. Filtration and evaporation of the solvent gave a residue that was purified by silica gel chromatography, eluting with 20% ethyl acetate in heptane, to give the title compound. MS (ESI) m/e 799.1 (M+H)⁺.

### 2.32.22. 2-(6-(tert-butoxycarbonyl)-5-(1-((3-(2-((tert-butoxycarbonyl)(methyl)amino)ethoxy)-5,7-dimethyltricyclo[3.3.1.1^{3,7}]dec-1-yl)methyl)-5-methyl-1H-pyrazol-4-yl)pyridin-2-yl)-1,2,3,4-tetrahydroisoquinoline-8-carboxylic acid

To a solution of Example 2.32.21 (10 g) in tetrahydrofuran (60 mL), methanol (30 mL) and water (30 mL) was added lithium hydroxide monohydrate (1.2 g). The mixture was stirred at room temperature for 24 hours. The reaction mixture was neutralized with 2% aqueous HCl and concentrated under vacuum. The residue was diluted with ethyl acetate (800 mL) and washed with water and brine, and dried over Na₂SO₄. Filtration and evaporation of the solvent gave the title compound. MS (ESI) m/e 785.1 (M+H)⁺.

### 2.32.23. tert-butyl 6-[8-(1,3-benzothiazol-2-ylcarbamoyl)-3,4-dihydroisoquinolin-2(1H)-yl]-3-(1-[(3-(2-[(tert-butoxycarbonyl)(methyl)amino]ethoxy)-5,7-dimethyltricyclo[3.3.1.1^{3,7}]dec-1-yl)methyl]-5-methyl-1H-pyrazol-4-yl pyridine-2-carboxylate

To a solution of Example 2.32.22 (10 g) in N,N-dimethylformamide (20 mL) was added benzo[d]thiazol-2-amine (3.24 g), fluoro-N,N,N′,N′-tetramethylformamidinium hexafluorophosphate (5.69 g) and N,N-diisopropylethylamine (5.57 g). The mixture was stirred at 60° C. for 3 hours. The reaction mixture was diluted with ethyl acetate (800 mL) and washed with water and brine, and dried over Na₂SO₄. Filtration and evaporation of the solvent gave a residue that was purified by silica gel chromatography, eluting with 20% ethyl acetate in dichloromethane, to give the title compound. MS (ESI) m/e 915.5 (M+H)⁺.

### 2.32.24. 6-[8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

To a solution of Example 2.32.23 (5 g) in dichloromethane (20 mL) was added trifluoroacetic acid (10 mL). The mixture was stirred overnight. The solvent was evaporated under vacuum, and the residue was dissolved in dimethyl sulfoxide/methanol (1:1, 10 mL), and chromatographed via reverse-phase using an Analogix system and a C18 cartridge (300 g), eluting with 10-85% acetonitrile and 0.1% trifluoroacetic acid in water, to give the title compound as a TFA salt. ¹H NMR (300 MHz, dimethyl sulfoxide dₜ) δ ppm 12.85 (s, 1H), 8.13-8.30 (m, 2H), 8.03 (d, 1H), 7.79 (d, 1H), 7.62 (d, 1H), 7.32-7.54 (m, 3H), 7.28 (d, 1H), 6.96 (d, 1H), 4.96 (dd, 1H), 3.80-3.92 (m, 4H), 3.48-3.59 (m, 1H), 2.91-3.11 (m, 2H), 2.51-2.59 (m, 4H), 2.03-2.16 (m, 2H), 1.21-1.49 (m, 6H), 0.97-1.20 (m, 4H), 0.87 (s, 6H). MS (ESI) m/e 760.4 (M+H)⁺.

### 2.32.25. 3-(1-((3-(2-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a solution of Example 2.32.24 (325 mg) and Example 2.32.7 (382 mg) in N,N-dimethylformamide (9 mL) at 0° C. was added N,N-diisopropylamine (49.1 mg). The reaction mixture was stirred at 0° C. for 5 hours, and acetic acid (22.8 mg) was added. The resulting mixture was diluted with ethyl acetate and washed with water and brine. The organic layer was dried over Na₂SO₄, filtered and concentrated. The residue was dissolved in a mixture of tetrahydrofuran (10 mL) and methanol (5 mL). To this solution at 0° C. was added 1 M aqueous lithium hydroxide solution (3.8 mL). The resulting mixture was stirred at 0° C. for 1 hour, acidified with acetic acid and concentrated. The concentrate was lyophilized to provide a powder. The powder was dissolved in N,N-dimethylformamide (10 mL), cooled in an ice-bath, and piperidine (1 mL) at 0° C. was added. The mixture was stirred at 0° C. for 15 minutes and 1.5 mL of acetic acid was added. The solution was purified by reverse-phase HPLC using a Gilson system, eluting with 30-80% acetonitrile in water containing 0.1% v/v trifluoroacetic acid, to provide the title compound. MS (ESI) m/e 1172.2 (M+H)⁺.

### 2.32.26. 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[6-(2,5-dioxo-2,5-dihydro-1H-pyrrol-1-yl)hexanoyl]-beta-alanyl}amino)phenyl beta-D-glucopyranosiduronic acid

To Example 2.32.25 (200 mg) in N,N-dimethylformamide (5 mL) at 0° C. was added 2,5-dioxopyrrolidin-1-yl 6-(2,5-dioxo-2,5-dihydro-1H-pyrrol-1-yl)hexanoate (105 mg) and N,N-diisopropylethylamine (0.12 mL). The mixture was stirred at 0° C. for 15 minutes, warmed to room temperature and purified by reverse-phase HPLC on a Gilson system using a 100 g C18 column, eluting with 30-80% acetonitrile in water containing 0.1% v/v trifluoroacetic acid, to provide the title compound. ¹H NMR (500 MHz, dimethyl sulfoxide-d₆) δ ppm 12.85 (s, 2H) 9.07 (s, 1H) 8.18 (s, 1H) 8.03 (d, 1H) 7.87 (t, 1H) 7.79 (d, 1H) 7.61 (d, 1H) 7.41-7.53 (m, 3H) 7.36 (q, 2H) 7.28 (s, 1H) 7.03-7.09 (m, 1H) 6.96-7.03 (m, 3H) 6.94 (d, 1H) 4.95 (s, 4H) 4.82 (t, 1H) 3.88 (t, 3H) 3.80 (d, 2H) 3.01 (t, 2H) 2.86 (d, 3H) 2.54 (t, 2H) 2.08 (s, 3H) 2.03 (t, 2H) 1.40-1.53 (m, 4H) 1.34 (d, 2H) 0.90-1.28 (m, 12H) 0.82 (d, 6H). MS (ESI) m/e 1365.3 (M+H)⁺.

### 2.33. Synthesis of Control Synthon 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[19-(2,5-dioxo-2,5-dihydro-1H-pyrrol-1-yl)-17-oxo-4,7,10,13-tetraoxa-16-azanonadecan-1-oyl]-beta-alanyl}amino)phenyl beta-D-glucopyranosiduronic acid (Synthon I)

The title compound was prepared using the procedure in Example 2.32.26, replacing 2,5-dioxopyrrolidin-1-yl 6-(2,5-dioxo-2,5-dihydro-1H-pyrrol-1-yl)hexanoate with 2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate. ¹H NMR (500 MHz, dimethyl sulfoxide-d₆) δ ppm 8.95 (s, 1H) 8.16 (s, 1H) 7.99 (d, 1H) 7.57-7.81 (m, 4H) 7.38-7.50 (m, 3H) 7.34 (q, 2H) 7.27 (s, 1H) 7.10 (d, 1H) 7.00 (d, 1H) 6.88-6.95 (m, 2H) 4.97 (d, 4H) 4.76 (d, 2H) 3.89 (t, 2H) 3.84 (d, 2H) 3.80 (s, 2H) 3.57-3.63 (m, 4H) 3.44-3.50 (m, 4H) 3.32-3.43 (m, 6H) 3.29 (t, 2H) 3.16 (q, 2H) 3.02 (t, 2H) 2.87 (s, 3H) 2.52-2.60 (m, 2H) 2.29-2.39 (m, 3H) 2.09 (s, 3H) 1.37 (s, 2H) 1.20-1.29 (m, 4H) 1.06-1.18 (m, 4H) 0.92-1.05 (m, 2H) 0.83 (s, 6H). MS (ESI) m/e 1568.6 (M−H)⁻.

### 2.34 Synthesis of 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

### 2.34.1 3-(1-((3-(2-((((2-(2-(2-aminoethoxy)ethoxy)-4-(((2R,3S,4R,5R,6R)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

To a cold (0° C.) solution of Example 2.11.6 (279 mg) and Example 1.14.9 (240 mg) in N,N-dimethylformamide (10 mL) was added N,N-diisopropylethylamine (0.157 mL). The reaction was slowly warmed to room temperature and was stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1233.0 (M−H)⁻.

### 2.34.2 3-(1-((3-(2-((((2-(2-(2-((R)-2-amino-3-sulfopropanamido)ethoxy)ethoxy)-4-(((2R,3S,4R,5R,6R)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

To a solution of (R)-2-((((9H-fluoren-9-yl)methoxy)carbonyl)amino)-3-sulfopropanoic acid (45.7 mg) in N,N-dimethylformamide (1 mL) was added O-(7-azabenzotriazol-1-yl)-N,N,N′,N′-tetramethyluronium hexafluorophosphate (45 mg) and N,N-diisopropylethylamine (0.02 mL). The mixture was stirred at room temperature for 10 minutes, and a solution of Example 2.34.1 (96 mg) and N,N-diisopropylethylamine (0.1 mL) in N,N-dimethylformamide (2 mL) was added. The reaction mixture was stirred at room temperature for 3 hours. To the reaction mixture was added diethylamine (0.1 mL), and the reaction was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. MS (ESI) m/e 1382.2 (M−H)⁻.

### 2.34.3 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.5.3, substituting Example 2.5.2 with Example 2.34.2. ¹H NMR (400 MHz, dimethyl sulfoxide-d₆) δ ppm 8.38 (s, 1H), 7.99 (d, 1H), 7.90-7.70 (m, 6H), 7.44 (s, 1H), 7.35 (t, 1H), 7.28 (d, 1H), 7.24-7.14 (m, 2H), 6.96 (s, 1H), 6.66 (s, 1H), 5.04 (s, 1H), 4.95 (s, 2H), 4.28 (q, 1H), 4.07 (d, 2H), 3.89 (dd, 3H), 3.22 (ddd, 6H), 2.87-2.61 (m, 4H), 2.20 (s, 3H), 2.04 (t, 2H), 1.93 (p, 2H), 1.54-0.90 (m, 20H), 0.83 (d, 7H). MS (ESI) m/e 1575.2 (M−H)⁻.

### 2.35 Synthesis of 2-[({[2-({3-[(4-{6-[5-(1,3-benzothiazol-2-ylcarbamoyl)quinolin-3-yl]-2-carboxypyridin-3-yl})-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

### 2.35.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a cold (0° C.) solution of Example 2.26.7 (76 mg) and 6-(5-(benzo[d]thiazol-2-ylcarbamoyl)quinolin-3-yl)-3-(1-((3,5-dimethyl-7-(2-(methylamino)ethoxy)adamantan-1-yl)methyl)-5-methyl-1H-pyrazol-4-yl)picolinic acid (62 mg) in N,N-dimethylformamide (2 mL) was added N,N-diisopropylethylamine (0.043 mL). The reaction was slowly warmed to room temperature and stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1183.3 (M−H)⁻.

### 2.35.2 3-(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a solution of (R)-2-((((9H-fluoren-9-yl)methoxy)carbonyl)amino)-3-sulfopropanoic acid (22.3 mg) in N,N-dimethylformamide (1 mL) was added O-(7-azabenzotriazol-1-yl)-N,N,N′,N′-tetramethyluronium hexafluorophosphate (22 mg) and N,N-diisopropylethylamine (0.02 mL). The mixture was stirred at room temperature for 10 minutes, and a solution of Example 2.35.1 (45 mg) and N,N-diisopropylethylamine (0.1 mL) in N,N-dimethylformamide(2 mL) was added. The reaction was stirred at room temperature for 3 hours. To the reaction mixture was added diethylamine (0.1 mL), and the reaction was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. MS (ESI) m/e 1334.5 (M−H)⁻.

### 2.35.3 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.34.1, substituting Example 2.5.2 with Example 2.35.2. ¹H NMR (400 MHz, dimethyl sulfoxide-d₆) δ ppm 9.72 (d, 1H), 9.43 (s, 1H), 8.32 (dd, 2H), 8.17 (d, 1H), 8.06 (d, 1H), 8.02-7.92 (m, 2H), 7.86 (d, 1H), 7.82-7.71 (m, 2H), 7.52-7.43 (m, 2H), 7.36 (t, 1H), 7.17 (d, 1H), 6.96 (s, 2H), 6.69 (d, 1H), 6.58 (dd, 1H), 5.03 (dd, 3H), 4.28 (q, 1H), 4.02 (d, 3H), 3.93 (d, 1H), 3.47-3.21 (m, 8H), 3.16 (p, 1H), 2.85 (d, 3H), 2.80-2.63 (m, 2H), 2.22 (s, 3H), 2.04 (t, 2H), 1.53-1.30 (m, 6H), 1.32-0.90 (m, 12H), 0.83 (d, 6H). MS (ESI) m/e 1527.4 (M−H)⁻.

### 2.36 Synthesis of 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl})oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

### 2.36.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid, Trifluoroacetic Acid

To a solution of Example 1.1.14 (157 mg) and Example 2.26.7 (167 mg) in N,N-dimethylformamide (3 mL) at 0° C. was added N,N-diisopropylethylamine (188 μL). The mixture was warmed to room temperature, stirred overnight and concentrated. The residue was dissolved in methanol (2 mL) and tetrahydrofuran (3 mL). The solution was cooled in an ice water bath and 1M aqueous lithium hydroxide solution (1.14 mL) was added. The mixture was stirred 0° C. at room temperature for 2 hours and concentrated. The residue was dissolved in dimethyl sulfoxide and purified by reverse-phase HPLC on a Gilson system (C18 column), eluting with 20-80% acetonitrile in water containing 0.1% trifluoroacetic acid, to provide the title compound.

### 2.36.2 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

To a solution of Example 2.36.1 (18 mg) and 2,5-dioxopyrrolidin-1-yl 6-(2,5-dioxo-2,5-dihydro-1H-pyrrol-1-yl)hexanoate (6.39 mg) in N,N-dimethylformamide (3 mL) was added N,N-diisopropylethylamine (24 μL). The resulting mixture was stirred for 1 hour and was purified by reverse-phase HPLC on a Gilson system (C18 column), eluting with 20-75% acetonitrile in water containing 0.1% trifluoroacetic acid, to provide the title compound. ¹H NMR (400 MHz, dimethyl sulfoxide-d₆) 8.36 (s, 1H), 7.97 (d, 1H), 7.85-7.70 (m, 4H), 7.43 (s, 1H), 7.38-7.30 (m, 1H), 7.26 (d, 1H), 7.23-7.10 (m, 2H), 6.95 (s, 2H), 6.65 (d, 1H), 6.56 (dd, 1H), 5.08-4.94 (m, 3H), 4.02 (dd, 2H), 3.92 (dd, 3H), 3.84 (s, 2H), 3.67 (t, 2H), 3.31-3.20 (m, 2H), 3.16 (q, 2H), 2.91-2.74 (m, 6H), 2.18 (s, 3H), 1.99 (t, 2H), 1.91 (p, 2H), 1.51-1.29 (m, 5H), 1.29-0.88 (m, 9H), 0.81 (d, 6H). MS (ESI) m/e 1380.2 (M−H)⁻.

### 2.37 Synthesis of 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

### 2.37.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared by substituting Example 1.6.3 for Example 1.12.10 and Example 2.11.6 for Example 2.26.7 in Example 2.26.8. MS (ESI) m/e 1182.3 (M−H)⁻.

### 2.37.2 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared by substituting Example 2.37.1 for Example 2.9.1 in Example 2.18.1. MS (ESI) m/e 1333.3 (M−H)⁻.

### 2.37.3 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{(2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared by substituting Example 2.37.2 for Example 2.9.1 in Example 2.9.2. ¹H NMR (400 MHz, dimethyl sulfoxide-d₆) δ ppm 9.02 (s, 1H), 8.37 (d, 1H), 8.23 (d, 1H), 8.20 (d, 1H), 8.18 (d, 1H), 8.06 (d, 1H), 8.01 (d, 1H), 7.94 (d, 1H), 7.87 (br d, 1H), 7.81 (d, 1H), 7.77 (brt, 1H), 7.70 (dd, 1H), 7.48 (dd, 1H), 7.48 (s, 1H), 7.37 (dd, 1H), 7.19 (d, 1H), 6.97 (s, 2H), 6.68 (d, 1H), 6.59 (dd, 1H), 5.06 (br m, 1H), 4.97 (s, 2H), 4.31 (m, 1H), 4.09 (m, 2H), 3.90 (m, 5H), 3.71 (m, 2H), 3.45 (m, 5H), 3.36 (m, 3H), 3.28 (m, 4H), 3.19 (m, 2H), 2.82 (br d, 2H), 2.76 (dd, 2H), 2.23 (s, 3H), 2.06 (t, 2H), 1.52-1.32 (m, 6H), 1.32-0.92 (m, 10H), 0.85 (br s, 6H). MS (ESI) m/e 1526.4 (M−H)⁻.

### 2.38 Synthesis of 2-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

### 2.38.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(4-(benzo[d]thiazol-2-ylcarbamoyl)quinolin-6-yl)picolinic acid

The title compound was prepared as described in Example 2.36.1, substituting Example 1.1.14 with Example 1.11.4.

### 2.38.2 2-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

The title compound was prepared as described in Example 2.36.2, substituting Example 2.36.1 with Example 2.38.1. ¹H NMR (400 MHz, dimethyl sulfoxide-d₆) δ 9.12 (d, 1H), 8.93 (s, 1H), 8.60 (dd, 1H), 8.27 (d, 1H), 8.21 (d, 1H), 8.07 (d, 1H), 7.97-7.90 (m, 2H), 7.81 (d, 2H), 7.47 (d, 2H), 7.37 (t, 1H), 7.17 (d, 1H), 6.96 (s, 2H), 6.67 (d, 1H), 6.58 (dd, 1H), 5.11-4.96 (m, 3H), 4.04 (dd, 2H), 3.92 (d, 1H), 3.86 (s, 2H), 3.40 (q, 5H), 3.34 (t, 2H), 3.31-3.22 (m, 4H), 3.17 (q, 2H), 2.85 (d, 3H), 2.20 (s, 3H), 2.00 (t, 2H), 1.51-1.31 (m, 6H), 1.30-0.88 (m, 13H), 0.82 (d, 6H). MS (ESI) m/e 1400.3 (M+Na)⁺.

### 2.39 Synthesis of 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

### 2.39.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

The title compound was prepared by substituting Example 1.1.14 for Example 1.12.10 and Example 2.11.6 for Example 2.26.7 in Example 2.26.8. MS (ESI−) m/e 1187.2 (M−H)⁻.

### 2.39.2 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

The title compound was prepared by substituting Example 2.39.1 for Example 2.9.1 in Example 2.18.1. MS (ESI−) m/e 1338.2 (M−H)⁻.

### 2.39.3 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{(2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared by substituting Example 2.39.2 for Example 2.9.1 in Example 2.9.2. ¹H NMR (400 MHz, dimethyl sulfoxide-d₆) δ ppm 8.39 (br s 1H), 8.00 (d, 1H), 7.86 (d, 2H), 7.81 (d, 1H), 7.77 (d, 2H), 7.48 (v br s, 1H), 7.46 (s, 1H), 7.37 (t, 1H), 7.29 (d, 1H), 7.23 (d, 1H), 7.19 (d, 1H), 6.92 (s, 2H), 6.68 (d, 1H), 6.59 (dd, 1H), 5.06 (br m, 1H), 4.97 (s, 2H), 4.31 (m, 1H), 4.09 (m, 2H), 3.96 (br t, 2H), 3.88 (br m, 2H), 3.71 (m, 2H), 3.45 (m, 5H), 3.37 (m, 3H), 3.28 (m, 4H), 3.18 (m, 2H), 2.86 (br m, 5H), 2.75 (dd, 2H), 2.22 (s, 3H), 2.06 (t, 2H), 1.95 (m, 2H), 1.52-1.32 (m, 6H), 1.32-0.92 (m, 12H), 0.85 (br s, 6H). MS (ESI−) m/e 1531.2 (M−H)⁻.

### 2.40 Synthesis of 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-(3-{[6-(2,5-dioxo-2,5-dihydro-1H-pyrrol-1-yl)hexanoyl]amino}propoxy)phenyl beta-D-glucopyranosiduronic acid

### 2.40.1 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

The title compound was prepared as described in Example 2.36.1, substituting Example 2.26.7 with Example 2.28.3. MS (ESI) m/e 1159.2 (M+H)⁺.

### 2.40.2 4-[({[2-(3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-(3-{[6-(2,5-dioxo-2,5-dihydro-1H-pyrrol-1-yl)hexanoyl]amino}propoxy)phenyl beta-D-glucopyranosiduronic acid

The title compound was prepared as described in Example 2.36.2, substituting Example 2.36.1 with Example 2.40.1. ¹H NMR (400 MHz, dimethyl sulfoxide-d₆) δ 8.38 (s, 1H), 7.98 (d, 1H), 7.87-7.72 (m, 2H), 7.44 (s, 1H), 7.35 (t, 1H), 7.28 (d, 1H), 7.19 (dd, 2H), 6.96 (s, 2H), 6.62 (d, 1H), 6.57 (dd, 1H), 5.03 (s, 1H), 4.95 (s, 2H), 4.03-3.81 (m, 8H), 3.42-3.20 (m, 7H), 3.16 (q, 2H), 2.90-2.75 (m, 5H), 2.20 (s, 3H), 2.01 (t, 2H), 1.97-1.87 (m, 2H), 1.80 (t, 2H), 1.45 (td, 4H), 1.13 (d, 8H), 0.83 (d, 6H). MS (ESI) m/e 1350.2 (M−H)⁻.

### 2.41 Synthesis of 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid

### 2.41.1 3-(1-((3-(2-((((2-(3-((R)-2-amino-3-sulfopropanamido)propoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

To a solution of (R)-2-((((9H-fluoren-9-yl)methoxy)carbonyl)amino)-3-sulfopropanoic acid (35.4 mg) and O-(7-azabenzotriazol-1-yl)-N,N,N,N′-tetramethyluronium hexafluorophosphate (29.8 mg) in N,N-dimethylformamide (1 mL) at 0° C. was added N,N-diisopropylethylamine (30 μL). The resulting mixture was stirred for 15 minutes and added to a mixture of Example 2.40.1 (70 mg) and N,N-diisopropylethylamine (80 μL) in N,N-dimethylformamide (2 mL). The resulting mixture was stirred for 1 hour. Diethylamine (62.2 μL) was added, and the mixture was stirred for 1 hour. The reaction was cooled in ice-bath and trifluoroacetic acid (93 μL) was added. The mixture was diluted with dimethyl sulfoxide (5.5 mL) and purified by reverse-phase HPLC on a Gilson system (C18 column), eluting with 20-75% acetonitrile in water containing 0. 1% trifluoroacetic acid, to provide the title compound.

### 2.41.2 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid

The title compound was prepared as described in Example 2.36.2, substituting Example 2.36.1 with Example 2.41.1. ¹H NMR (501 MHz, dimethyl sulfoxide-d₆) δ 8.37 (s, 1H), 7.98 (d, 1H), 7.87-7.72 (m, 5H), 7.44 (s, 1H), 7.35 (t, 1H), 7.27 (d, 1H), 7.20 (t, 1H), 7.16 (d, 1H), 6.96 (s, 2H), 6.63 (d, 1H), 6.55 (dd, 1H), 5.02 (s, 1H), 4.95 (s, 2H), 4.26 (q, 1H), 4.04-3.79 (m, 8H), 3.32-3.08 (m, 4H), 2.89-2.66 (m, 7H), 2.35 (q, 0H), 2.20 (s, 3H), 2.03 (t, 2H), 1.93 (p, 2H), 1.80 (t, 2H), 1.52-1.30 (m, 6H), 1.30-0.89 (m, 13H), 0.83 (d, 6H). MS (ESI) m/e 1502.2 (M−H)⁻.

### 2.42 Synthesis of 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl})amino)ethoxy]ethoxy}phenyl beta-D-glucopyranosiduronic acid

### 2.42.1 3-(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

The title compound was prepared as described in Example 2.41.1, substituting Example 2.40.1 with Example 2.36.1. MS (ESI) m/e 1338.2 (M−H)⁻.

### 2.42.2 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.36.2, substituting Example 2.36.1 with Example 2.42.1. ¹H NMR (500 MHz, dimethyl sulfoxide-d₆) δ 8.39 (s, 1H), 8.00 (d, 1H), 7.86 (t, 2H), 7.83-7.73 (m, 3H), 7.45 (s, 1H), 7.40-7.32 (m, 1H), 7.29 (d, 1H), 7.26-7.13 (m, 2H), 6.97 (s, 2H), 6.70 (d, 1H), 6.59 (dd, 1H), 5.11-4.94 (m, 3H), 4.29 (dt, 1H), 4.04 (dd, 2H), 3.99-3.91 (m, 3H), 3.87 (d, 2H), 3.69 (t, 2H), 3.40-3.07 (m, 7H), 2.91-2.74 (m, 6H), 2.69 (dd, 1H), 2.21 (s, 3H), 2.05 (t, 21H), 1.94 (p, 2H), 1.53-1.32 (m, 5H), 1.31-0.90 (m, 7H), 0.84 (d, 6H). MS (ESI) m/e 1531.2 (M−H)⁻.

### 2.43 Synthesis of 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

### 2.43.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.34.1, substituting Example 2.5.2 with Example 1.15.1. MS (ESI) m/e 1228.1 (M−H)⁻.

### 2.43.2 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrzol-4-yl)-6-(8-(benzo[d]thiazol-2-ylcarbamoyl)naphthalen-2-yl)picolinic acid

The title compound was prepared as described in Example 2.34.2, substituting Example 2.34.1 with Example 2.43.2. MS (ESI) m/e 1379.1.1 (M+H)⁺.

### 2.43.3 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.34, substituting Example 2.34.2 with Example 2.43.2. ¹H NMR (400 MHz, dimethyl sulfoxide-d) δ ppm 9.00 (s, 1H), 8.36 (d, 1H), 8.27-8.12 (m, 3H), 8.05 (d, 1H), 8.00 (d, 1H), 7.92 (d, 1H), 7.85 (d, 1H), 7.79 (d, 1H), 7.75 (t, 1H), 7.69 (t, 1H), 7.52-7.43 (m, 2H), 7.35 (t, 1H), 7.24-7.12 (m, 1H), 6.95 (s, 2H), 6.66 (s, 1H), 6.57 (d, 1H), 5.04 (d, 1H), 4.95 (s, 2H), 4.29 (q, 1H), 4.15-4.01 (m, 2H), 3.86 (d, 3H), 3.46-3.11 (m, 16H), 2.84-2.62 (m, 2H), 2.21 (d, 3H), 2.04 (t, 2H), 1.53-1.30 (m, 6H), 1.28-0.89 (m, 6H), 0.82 (d, 7H). MS (ESI) m/e 1570.4 (M−H)⁻.

### 2.44 Synthesis of N-[6-(2,5-dioxo-2,5-dihydro-1H-pyrrol-1-yl)hexanoyl]-L-valyl-N-[4-({[{2-[{8-(1,3-benzothiazol-2-ylcarbamoyl)-2-[6-carboxy-5-(1-{[3-(2-methoxyethoxy)-5,7-dimethyltricyclo[3.3.1.1^{3,7}]dec-1-yl]methyl}-5-methyl-1H-pyrazol-4-yl)pyridin-2-yl]-1,2,3,4-tetrahydroisoquinolin-6-yl}(methyl)amino]ethyl}(methyl)carbamoyl]oxy}methyl)phenyl]-L-alaninamide

The title compound was prepared as described in Example 2.30, substituting Example 1.17.10 with Example 1.21.12. MS (ESI) m/e 1359.5 (M+H)⁺, 1357.5 (M−H)⁻.

### 2.45 Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-6-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-L-alaninamide

The title compound was prepared as described in Example 2.30, substituting Example 1.17.10 with Example 1.22.9. MS (ESI) m/e 1302.5 (M+H)⁺, 1300.5 (M−H)⁻.

### 2.46 Synthesis of 2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

### 2.46.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.43.1, substituting Example 2.11.6 with Example 2.26.7. MS (ESI) m/e 1228.1 (M−H)⁻.

### 2.46.2 3-(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.34.2, substituting Example 2.34.1 with Example 2.46.1. MS (ESI) m/e 1377.5 (M−H)⁻.

### 2.46.3 2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.34, substituting Example 2.34.2 with Example 2.46.2. ¹H NMR (501 MHz, dimethyl sulfoxide-d₆) δ ppm 13.08 (s, 1H), 9.00 (s, 1H), 8.36 (d, 1H), 8.25-8.12 (m, 3H), 8.05 (d, 1H), 8.00 (d, 1H), 7.92 (d, 1H), 7.85 (d, 1H), 7.78 (dd, 2H), 7.72-7.65 (m, 1H), 7.50-7.43 (m, 2H), 7.35 (t, 1H), 7.21-7.14 (m, 1H), 6.96 (s, 2H), 6.69 (d, 1H), 6.58 (d, 1H), 5.13-4.93 (m, 3H), 4.28 (q, 1H), 4.03 (dd, 2H), 3.94 (d, 1H), 3.86 (d, 2H), 3.67 (t, 2H), 3.31-3.08 (m, 8H), 2.83-2.64 (m, 2H), 2.21 (d, 3H), 2.04 (t, 2H), 1.53-1.30 (m, 5H), 1.30-0.89 (m, 11H), 0.89-0.75 (m, 6H). MS (ESI) m/e 1570.5 (M−H)⁻.

### 2.47 Synthesis of 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

### 2.47.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

The title compound was prepared as described in Example 2.36.1, substituting Example 1.1.14 with Example 1.10.3.

### 2.47.2 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

The title compound was prepared as described in Example 2.36, substituting Example 2.36.1 with Example 2.47.1. ¹H NMR (501 MHz, dimethyl sulfoxide-d₆) δ 13.17 (s, 1H), 9.70 (d, 1H), 9.39 (s, 1H), 8.31 (dd, 2H), 8.16 (d, 1H), 8.06 (dd, 1H), 8.01-7.90 (m, 2H), 7.83-7.71 (m, 2H), 7.52-7.43 (m, 2H), 7.39-7.31 (m, 1H), 7.18 (d, 1H), 6.96 (s, 2H), 6.65 (d, 1H), 6.58 (dd, 1H), 5.04 (s, 1H), 4.96 (s, 2H), 4.09 (dtd, 2H), 3.87 (s, 2H), 3.70 (t, 2H), 3.40-3.14 (m, 7H), 2.85 (d, 3H), 2.22 (s, 3H), 2.01 (t, 2H), 1.49-1.30 (m, 6H), 1.30-0.90 (m, 10H), 0.90-0.74 (m, 6H). MS (ESI) m/e 1400.4 (M+Na)⁺.

### 2.48 Synthesis of 4-[({[2-({3-[(4-{6-[5-(1,3-benzothiazol-2-ylcarbamoyl)quinolin-3-yl]-2-carboxypyridin-3-yl})-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[6-(2,5-dioxo-2,5-dihydro-1H-pyrrol-1-yl)hexanoyl]amino}ethoxy)ethoxy]phenyl beta-D-glucopyranosiduronic acid

### 2.48.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a solution of Example 1.10.3 (208 mg) and Example 2.11.6 (267 mg) in N,N-dimethylformamide (2 mL) at 0° C. was added N,N-diisopropylethylamine (251 μL). The resulting mixture was stirred at room temperature overnight and concentrated. The residue was dissolved in methanol (3 mL) and tetrahydrofuran (5 mL). The solution was cooled in an ice water bath and 1M aqueous lithium hydroxide solution was added (2.87 mL). The mixture was stirred at 0° C. for 2 hours and was acidified with trifluoroacetic acid. The reaction mixture was concentrated under reduced pressure. The residue was diluted with dimethyl sulfoxide and purified by reverse-phase HPLC on a Gilson system (C18 column), eluting with 20-75% acetonitrile in water containing 0.1% trifluoroacetic acid, to provide the title compound. MS (ESI) m/e 1185.1 (M+H)⁺.

### 2.48.2 4-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[6-(2,5-dioxo-2,5-dihydro-1H-pyrrol-1-yl)hexanoyl]amino}ethoxy)ethoxy]phenyl beta-D-glucopyranosiduronic acid

The title compound was prepared as described in Example 2.36.2, substituting Example 2.36.1 with Example 2.48.1. ¹H NMR (400 MHz, dimethyl sulfoxide-d₆) δ 13.18 (s, 1H), 9.70 (d, 1H), 9.39 (s, 1H), 8.31 (dd, 2H), 8.16 (d, 1H), 8.06 (d, 1H), 8.01-7.90 (m, 2H), 7.80 (d 2H), 7.52-7.43 (m, 2H), 7.39-7.32 (m, 1H), 7.18 (d, 1H), 6.96 (s, 2H), 6.67 (d, 1H), 6.58 (dd, 1H), 5.11-4.90 (m, 3H), 4.03 (d, 2H), 3.95-3.82 (m, 3H), 3.68 (t, 2H), 3.48-3.23 (m, 10H), 3.18 (t, 2H), 2.85 (d, 3H), 2.22 (s, 3H), 2.00 (t, 2H), 1.51-1.31 (m, 5H), 1.19 (dd, 10H), 0.83 (d, 6H). MS (ESI) m/e 1376.4 (M−H)⁻.

### 2.49 Synthesis of 6-[5-(1,3-benzothiazol-2-ylcarbamoyl)quinolin-3-yl]-3-(1-{[3-(2-{[6-(2,5-dioxo-2,5-dihydro-1H-pyrrol-1-yl)hexanoyl](methyl)amino}ethoxy)-5,7-dimethyltricyclo[3.3.1.1^{3,7}]dec-1-yl]methyl}-5-methyl-1H-pyrazol-4-yl)pyridine-2-carboxylic acid

The title compound was prepared as described in Example 2.36.2, substituting Example 2.36.1 with Example 1.10.3. ¹H NMR (400 MHz, dimethyl sulfoxide-d₆) δ 13.21 (s, 1H), 9.70 (d, 1H), 9.40 (s, 1H), 8.42-8.27 (m, 2H), 8.16 (d, 1H), 8.06 (d, 1H), 8.04-7.90 (m, 2H), 7.80 (d, 1H), 7.56-7.44 (m, 2H), 7.42-7.31 (m, 1H), 6.95 (d, 2H), 3.87 (s, 2H), 3.55-3.18 (m, 5H), 2.95 (s, 1H), 2.76 (s, 2H), 2.28 (t, 1H), 2.22 (s, 4H), 1.53-1.29 (m, 6H), 1.28-0.91 (m, 10H), 0.84 (s, 6H). MS (ESI) m/e 949.1 (M+H)⁺.

### 2.50 Synthesis of 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[3-(2,5-dioxo-2,5-dihydro-1H-pyrrol-1-yl)propanoyl]-beta-alanyl}amino)phenyl beta-D-glucopyranosiduronic acid

### 2.50.1 3-(1-((3-(2-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

The title compound was prepared by substituting Example 1.27.4 for Example 2.32.24 in Example 2.32.25. MS (ESI) m/e: 1156.6 (M+H)⁺.

### 2.50.2 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[3-(2,5-dioxo-2,5-dihydro-1H-pyrrol-1-yl)propanoyl]-beta-alanyl}amino)phenyl beta-D-glucopyranosiduronic acid

The title compound was prepared by substituting Example 2.50.1 for Example 2.11.7 in Example 2.11.8. ¹H NMR (501 MHz, dimethyl sulfoxide-d) δ ppm 13.00 (s, 2H); 9.06 (s, 1H), 8.29 (dd, 1H), 8.22 (d, 1H), 8.18 (s, 1H), 8.04 (t, 2H), 7.97 (d, 1H), 7.90 (d, 1H), 7.79 (d, 1H), 7.50-7.43 (m, 3H), 7.35 (ddd, 1H), 7.25 (t, 1H), 7.06 (d, 1H), 7.01 (dd, 1H), 6.94 (s, 2H), 4.96 (s, 2H), 4.81 (s, 1H), 3.33-3.25 (m, 6H), 2.87 (d, 3H), 2.50 (d, 3H), 2.31 (dd, 2H), 2.21 (s, 3H), 1.38 (d, 2H), 1.30-0.77 (m, 18H). MS (ESI) m/e 1305.2 (M−H)⁻.

### 2.51 Synthesis of 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

### 2.51.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

The title compound was prepared by substituting Example 1.27.4 for Example 1.12.10 in Example 2.11.7. MS (ESI) m/e: 1172.9 (M+H)⁺.

### 2.51.2 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

The title compound was prepared by substituting Example 2.5 1.1 for Example 2.11.7 in Example 2.11.8. ¹H NMR (501 MH-z, dimethyl sulfoxide-d₆) δ ppm 11.16 (s, 2H), 8.27 (d, 1), 8.19 (d, 1H), 8.06-7.94 (m, 3H), 7.88 (d, 1H), 7.77 (d, 1H), 7.50-7.39 (m, 3H), 7.33 (t, 1H), 7.26-7.13 (m, 2H), 6.93 (s, 2H), 6.63 (d, 1H), 6.57 (dd, 1H), 5.03 (d, 1H), 4.94 (s, 2H), 4.13-4.00 (m, 2H), 3.86 (d, 3H), 3.14 (q, 2H), 2.83 (d, 3H), 2.29 (t, 2H), 2.20 (s, 3H), 1.36 (d, 2H), 1.28-0.73 (m, 16H). MS (ESI) m/e 1322.4 (M−H)⁻.

### 2.52 Synthesis of 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[3-(2 5-dioxo-2,5-dihydro-1H-pyrrol-1-yl)propanoyl]-3-sulfo-L-alanyl}amino)ethoxy]ethoxy}phenyl beta-D-glucopyranosiduronic acid

### 2.52.1 3-(1-((3-(2-((((2-(2-(2-((R)-2-amino-3-sulfopropanamido)ethoxy)ethoxy)-4-(((2S,3R,4S,5S,6S)-6-carboxy-3,45-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

The title compound was prepared by substituting Example 2.51.1 for Example 2.9.1 in Example 2.18.1. MS (ESI) m/e: 1325.5 (M+H)⁺.

### 2.52.2 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid

The title compound was prepared by substituting Example 2.52.1 for Example 2.11.7 in Example 2.11.8. ¹H NMR (501 MHz, dimethyl sulfoxide-d₆) δ ppm 11.17 (s, 2H), 8.27 (d, 1H), 8.20 (d, 1H), 8.03 (dd, 2H), 7.96 (d, 1H), 7.89 (d, 1H), 7.82-7.75 (m, 2H), 7.50 (s, 1H), 7.48-7.41 (m, 2H), 7.34 (t, 1H), 7.24 (t, 1H), 7.18 (d, 1H), 6.93 (s, 2H), 6.66 (d, 1H), 6.58 (dd, 1H), 5.04 (d, 1H), 4.95 (s, 2H), 3.70 (t, 2H), 3.58 (t, 2H), 3.48-3.14 (m, 11H), 2.89-2.79 (m, 4H), 2.73 (dd, 1H), 2.37 (m, 2H), 2.21 (s, 3H), 1.45-0.73 (m, 19H). MS (ESI) m/e 1473.3 (M−H)⁻.

### 2.53 Synthesis of 4-[({[2-({3-[(4-{6-[7-(1,3-benzothiazol-2-ylcarbamoyl)-3-methyl-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

### 2.53.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-3-methyl-1H-indol-2-yl)picolinic acid

The title compound was prepared by substituting Example 1.29.7 for Example 1.12.10 in Example 2.11.7. MS (ESI) m/e: 1187.1 (M+H)⁺.

### 2.53.2 4-[({[2-({3-[(4-{6-[7-(1,3-benzothiazol-2-ylcarbamoyl)-3-methyl-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

The title compound was prepared by substituting Example 2.53.1 for Example 2.11.7 in Example 2.11.8. ¹H NMR (501 MHz, dimethyl sulfoxide-d₆) δ ppm 11.01 (s, 1H), 8.28 (d, 1H), 8.06-7.94 (m, 4H), 7.91 (d, 1H), 7.76 (d, 1H), 7.50-7.42 (m, 2H), 7.32 (td, 1H), 7.26-7.15 (m, 2H), 6.93 (s, 2H), 6.64 (d, 1H), 6.58 (dd, 1H), 5.03 (d, 1H), 4.95 (s, 2H), 4.11-3.99 (m, 2H), 3.87 (d, 3H), 3.68 (t, 2H), 3.56 (dd, 2H), 3.47-3.33 (m, 5H), 3.33-3.19 (m, 4H), 3.14 (q, 2H), 2.84 (d, 3H), 2.63 (s, 3H), 2.30 (dd, 2H), 2.21 (s, 3H), 1.42-0.72 (m, 21H). MS (ESI) m/e 1336.3 (M−H)⁻.

### 2.54 Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)isoquinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide

The title compound was prepared as described in Example 2.2, substituting Example 1.3.2 with Example 1.26.10. ¹H NMR (400 MHz, dimethyl sulfoxide-d₆) δ ppm 13.28 (s, 2H), 9.96 (s, 1H), 9.59 (s, 1H), 9.03 (d, 2H), 8.53 (d, 1H), 8.42 (d, 1H), 8.25 (d, 1H), 8.05 (t, 2H), 7.97 (d, 1H), 7.78 (dd, 2H), 7.58 (d, 2H), 7.47 (d, 2H), 7.36 (t, 1H), 7.26 (d, 2H), 6.97 (s, 2H), 5.96 (s, 1H), 4.96 (s, 2H), 4.45-4.29 (m, 1H), 4.17 (t, 1H), 3.51-3.18 (m, 6H), 3.07-2.75 (m, 4H), 2.22 (s, 3H), 2.11 (dq, 1H), 2.02-1.82 (m, 1H), 1.76-0.88 (m, 18H), 0.81 (dd, 14H). MS (ESI) m/e 1352.4 (M−H)⁻.

### 2.55 Synthesis of 4-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyloxy}methyl]-3-[2-(2-{[(2,5-dioxo-2,5-dihydro-1H-pyrrol-1-yl)acetyl]amino}ethoxy)ethoxy]phenyl beta-D-glucopyranosiduronic acid

### 2.55.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(1-(benzo[d]thiazol-2-ylcarbamoyl)-5,6-dihydroimidazo[1,5-a]pyrazin-7(8H)-yl)picolinic acid

The title compound was prepared by substituting Example 1.4.10 for Example 1.12.10 in Example 2.11.7. MS (ESI) m/e 1165 (M+H)⁺, 1163 (M−H)⁻.

### 2.55.2 4-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-3-[2-(2-{[(2,5-dioxo-2,5-dihydro-1H-pyrrol-1-yl)acetyl]amino}ethoxy)ethoxy]phenyl beta-D-glucopyranosiduronic acid

The title compound was prepared by substituting Example 2.55.1 for Example 2.9.1 in Example 2.10. ¹H NMR (300 MHz, dimethyl sulfoxide-d₆) δ ppm 8.22 (t, 1H), 8.05 (s, 1H), 7.99 (d, 1H), 7.76 (d, 1H), 7.61 (d, 1H), 7.46 (t, 1H), 7.35-7.31 (m, 2H), 7.20 (d, 1H), 7.15 (d, 1H), 7.07 (s, 2H), 6.66 (d, 1H), 6.61 (dd, 1H), 5.12 (s, 2H), 5.08 (d, 1H), 4.94 (s, 2H), 4.28 (t, 2H), 4.09 (m, 4H), 4.03 (s, 2H), 3.91 (m, 3H), 3.84 (m, 4H), 3.73 (t, 2H), 3.49 (t, 2H), 3.40 (t, 2H), 3.34 (m, 2H), 3.30 (dd, 2H), 3.26 (m, 2H), 3.06 (q, 2H), 2.13 (s, 3H), 1.39 (bs, 2H), 1.26 (q, 4H), 1.13 (q, 4H), 1.02 (q, 2H), 0.85 (s, 6H). MS (ESI) m/e 1302 (M+H)⁺.

### 2.56 Synthesis of 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-4-[19-(2,5-dioxo-2,5-dihydro-1H-pyrrol-1-yl)-14-oxo-4,7,10-trioxa-13-azanonadec-1-yl]phenyl beta-D-glucopyranosiduronic acid

### 2.56.1 3-(1-((3-(2-((((5-(3-(2-(2-(2-aminoethoxy)ethoxy)ethoxy)propyl)-2-(((3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a cold (0° C.) solution of(3R,4S,5S,6S)-2-(4-(1-(9H-fluoren-9-yl)-3-oxo-2,7,10,13-tetraoxa-4-azahexadecan-16-yl)-2-((((4-nitrophenoxy)carbonyl)oxy)methyl)phenoxy)-6-(methoxycarbonyl)tetrahydro-2H-pyran-3,4,5-triyl triacetate (56 mg) and Example 1.43.5 (47 mg) in N,N-dimethylformamide (2 mL) was added N,N-diisopropylethylamine (0.026 mL). The reaction was slowly warmed to room temperature and stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1255.4 (M−H)⁻.

### 2.56.2 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-4-[19-(2,5-dioxo-2,5-dihydro-1H-pyrrol-1-yl)-1 4-oxo-4,7,10-trioxa-13-azanonadec-1-yl]phenyl beta-D-glucopyranosiduronic acid

To a solution of Example 2.56.1 (21 mg) in N,N-dimethylformamide (2 mL) was added 2,5-dioxopyrrlidin-1-yl 6-(2,5-dioxo-2,5-dihydro-1H-pyrrol-1-yl)hexanoate (5.24 mg) and N,N-diisopropylethylamine (0.012 mL). The reaction mixture was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. ¹H NMR (400 MHz, dimethyl sulfoxide-d₆) δ ppm 13.17 (s, 2H), 9.68 (d, 1H), 9.37 (s, 1H), 8.29 (dd, 2H), 8.14 (d, 1H), 8.04 (d, 1H), 8.01-7.88 (m, 2H), 7.82-7.69 (m, 2H), 7.51-7.40 (m, 2H), 7.38-7.29 (m, 1H), 7.17 (t, 1H), 7.13-7.01 (m, 2H), 6.95 (s, 3H), 5.02 (s, 2H), 4.94-4.86 (m, 1H), 3.91-3.79 (m, 4H), 3.33 (td, 9H), 3.29-3.22 (m, 2H), 3.12 (q, 2H), 3.04 (d, 2H), 2.20 (s, 3H), 1.98 (t, 2H), 1.70 (p, 2H), 1.42 (dt, 7H), 1.31-0.89 (m, 13H), 0.82 (s, 7H). MS (ESI) m/e 1448.3 (M−H)⁻.

### 2.57 Synthesis of 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[4-({N-[6-(2,5-dioxo-2,5-dihydro-1H-pyrrol-1-yl)hexanoyl]-3-sulfo-L-alanyl}amino)butyl]phenyl beta-D-glucopyranosiduronic acid

### 2.57.1 (2S,3R,4S,5S,6S)-2-(3-bromo-4-formylphenoxy)-6-(methoxycarbonyl)tetrahydro-2H-pyran-3,4,5-triyl triacetate

A mixture of (3R,4S,5S,6S)-2-bromo-6-(methoxycarbonyl)tetrahydro-2H-pyran-3,4,5-triyl triacetate (2.67 g), 2-bromo-4-hydroxybenzaldehyde (0.90 g) and silver oxide (1.56 g) was stirred in acetonitrile (20 mL) at room temperature protected from light. After 3 hours, the reaction was diluted with dichloromethane (20 mL), filtered through diatomaceous earth, washed with additional dichloromethane (40 mL) and concentrated. The residue was purified by silica gel chromatography, eluting with a gradient of 5% to 50% hexanes/ethyl acetate over 30 minutes, to provide the title compound. MS (ESI) m/e 517.1 (M+H)⁺.

### 2.57.2 (9H-fluoren-9-yl)methyl but-3-yn-1-ylcarbamate

A solution of but-3-yn-1-amine hydrochloride (9 g) and N-ethyl-N-isopropylpropan-2-amine (44.7 mL) was stirred in dichloromethane (70 mL) and the mixture was cooled to 0° C. A solution of (9H-fluoren-9-yl)methyl carbonochloridate (22.06 g) in dichloromethane (35 mL) was added, and the reaction was stirred for 2 hours. The reaction mixture was concentrated. The crude material was deposited onto silica gel, loaded onto a silica gel column and eluted with petroleum diethyl ether/ethyl acetate (10%-25%) to provide the title compound. MS (ESI) m/e 314 (M+Na)⁺.

### 2.57.3 (2S,3R,4S,5S,6S)-2-(3-(4-((((9H-fluoren-9-yl)methoxy)carbonyl)amino)but-1-yn-1-yl)-4-formylphenoxy)-6-(methoxycarbonyl)tetrahydro-2H-pyran-3,4,5-triyl triacetate

Example 2.57.1 (0.389 g), Example 2.57.2 (0.285 g), bis(triphenylphosphsine)palladium(II) dichloride (0.053 g), and copper(I) iodide (0.014 g) were weighed into a vial and the vial was flushed with a stream of nitrogen. N,N-diisopropylethylamine (0.263 mL) and N,N-dimethylformamide (1.5 mL) were added, and the reaction was stirred at room temperature overnight. The reaction mixture was diluted with diethyl ether (50 mL) and washed with water (30 mL) and brine (30 mL). The organic layer was dried over magnesium sulfate, filtered, and concentrated. The residue was purified by silica gel chromatography, eluting with a gradient of 5% to 60% ethyl acetate/heptanes over 30 minutes, to provide the title compound. MS (ESI) m/e 728.4 (M+H)⁺.

### 2.57.4 (2S,3R,4S,5S,6S)-2-(3-(4-((((9H-fluoren-9-yl)methoxy)carbonyl)amino)butyl)-4-formylphenoxy)-6-(methoxycarbonyl)tetrahydro-2H-pyran-3,4,5-triyl triacetate

Example 2.57.3 (262 mg) and tetrahydrofuran (10 mL) were added to 10% palladium/C (50 mg) in a 50 mL pressure bottle and the mixture was shaken for 2 hours at room temperature under 30 psi H₂. The reaction mixture was filtered and concentrated to provide the title compound. MS (ESI) m/e 732.5 (M+H)⁺.

### 2.57.5 (2S,3R,4S,5S,6S)-2-(3-(4-((((9H-fluoren-9-yl)methoxy)carbonyl)amino)butyl)-4-(hydroxymethyl)phenoxy)-6-(methoxycarbonyl)tetrahydro-2H-pyran-3,4,5-triyl triacetate

A solution of Example 2.57.4 (0.235 g) in tetrahydrofuran (1.0 mL) and methanol (1.0 mL) was cooled to 0° C. and sodium borohydride (6.07 mg) was added in one portion. The reaction was stirred for 15 minutes and was diluted with ethyl acetate (75 mL) and water (50 mL). The organic layer was separated, washed with brine (50 mL), dried over magnesium sulfate, filtered, and concentrated. The residue was purified by silica gel chromatography, eluting with a gradient of 10% to 70% ethyl acetate/heptanes, to provide the title compound. MS (ESI) m/e 734.5 (M+H)⁺.

### 2.57.6 (2S,3R,4S,5S,6S)-2-(3-(4-((((9H-fluoren-9-yl)methoxy)carbonyl)amino)butyl)-4-((((4-nitrophenoxy)carbonyl)oxy)methyl)phenoxy)-6-(methoxycarbonyl)tetrahydro-2H-pyran-3,4,5-triyl triacetate

To an ambient solution of Example 2.57.5 (0.148 g) and bis(4-nitrophenyl) carbonate (0.123 g) in N,N-dimethylformamide (1.5 mL) was added N,N-diisopropylethylamine (0.053 mL). After 3 hours, the reaction mixture was concentrated. The residue was purified by silica gel chromatography, eluting with a gradient of 10% to 60% ethyl acetate/hexanes, to provide the title compound. MS (ESI) m/e 899.5 (M+H)⁺.

### 2.57.7 3-(1-((3-(2-((((2-(4-aminobutyl)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a solution of Example 1.6.3 (0.101 g) and Example 2.57.6 (0.095 g) in N,N-dimethylformamide (1.0 mL) was added N,N-diisopropylethylamine (0.055 mL), and the reaction was stirred at room temperature for 3 hours. The reaction was quenched with a mixture of 2,2,2-trifluoroacetic acid (0.204 mL), water (1 mL) and N,N-dimethylformamide (1 mL) and was purified by preparatory reverse-phase HPLC on a Gilson 2020 system using a gradient of 5% to 50% acetonitrile water over 30 minutes. The product-containing fractions were lyophilized to provide the title compound. MS (ESI) m/e 1152.7 (M+H)⁺.

### 2.57.8 3-(1-((3-(2-((((2-(4-((R)-2-amino-3-sulfopropanamido)butyl)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a stirred solution of (R)-2-((((9H-fluoren-9-yl)methoxy)carbonyl)amino)-3-sulfopropanoic acid (0.058 g) and O-(7-azabenzotriazol-1-yl)-N,N,N′,N′-tetramethyluronium hexafluorophosphate (0.054 g) in N,N-dimethylformamide (0.5 mL) was added N,N-diisopropylethylamine (0.051 mL). After stirring for 5 minutes, the mixture was added to a mixture of Example 2.57.7 (0.113 g) and N,N-diisopropylethylamine (0.051 mL) in N,N-dimethylformamide (0.5 mL). After stirring for 2 hours, diethylamine (0.102 mL) was added, and the reaction mixture was stirred for 30 minutes. The reaction mixture was diluted with a solution of 2,2,2-trifluoroacetic acid (0.189 mL) in water (1 mL) and was purified by preparatory reverse-phase HPLC on a Gilson 2020 system using a gradient of 5% to 85% acetonitrile water over 30 minutes. The product-containing fractions were lyophilized to provide the title compound. MS (ESI) m/e 1303.1 (M+H)⁺.

### 2.57.9 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[4-({N-[6-(2,5-dioxo-2,5-dihydro-1H-pyrrol-1-yl)hexanoyl]-3-sulfo-L-alanyl}amino)butyl]phenyl beta-D-glucopyranosiduronic acid

To a solution of Example 2.57.8 (0.044 g) and 2,5-dioxopyrrolidin-1-yl 6-(2,5-dioxo-2,5-dihydro-1H-pyrrol-1-yl)hexanoate (0.012 g) in N,N-dimethylformamide (0.4 mL) was added N,N-diisopropylethylamine (0.027 mL), and the reaction mixture was stirred for 2 hours at room temperature. The reaction mixture was quenched with a mixture of 2,2,2-trifluoroacetic acid (0.060 mL), water (1 mL) and N,N-dimethylformamide (1 mL) and purified by preparatory reverse-phase HPLC on a Gilson 2020 system using a gradient of 5% to 50% acetonitrile water over 30 minutes. The product-containing fractions were lyophilized to provide the title compound. ¹H NMR (400 MHz, dimethyl sulfoxide-d₆) δ 13.10 (s, 1H), 9.02 (s, 1H), 8.38 (dd, 1H), 8.27-8.14 (m, 3H), 8.07 (d, 1H), 8.02 (d, 1H), 7.94 (d, 1H), 7.82 (dd, 2H), 7.79-7.66 (m, 2H), 7.53-7.44 (m, 1H), 7.48 (s, 1H), 7.37 (t, 1H), 7.23 (d, 1H), 6.98 (s, 2H), 6.88 (d, 1H), 6.82 (dd, 1H), 5.04 (d, 1H), 5.00 (s, 2H), 4.29 (q, 2H), 3.57 (s, 2H), 3.44 (s, 4H), 3.41 (d, 1H), 3.40-3.27 (m, 3H), 3.30-3.21 (m, 2H), 3.03 (t, 2H), 2.85 (s, 3H), 2.79 (dd, 1H), 2.70 (dd, 1H), 2.58 (s, 2H), 2.23 (s, 3H), 2.06 (t, 2H), 1.53-1.41 (m, 5H), 1.42 (s, 6H), 1.26 (s, 2H), 1.25-1.07 (m, 8H), 0.85 (s, 6H). MS (ESI) m/e 1494.1 (M−H)⁻.

### 2.58 Synthesis of 2-{6-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]-2-methyl-3,3-dioxido-7-oxo-8-oxa-3lambda⁶-thia-2,6-diazanonan-9-yl}-5-(4-{[(2,5-dioxo-2,5-dihydro-1H-pyrrol-1-yl)acetyl]amino}butyl)phenyl beta-D-glucopyranosiduronic acid

### 2.58.1 (9H-fluoren-9-yl)methyl but-3-yn-1-ylcarbamate

A solution of but-3-yn-1-amine hydrochloride (9 g) and N,N-diisopropylethylamine (44.7 mL) was stirred in dichloromethane (70 mL) and the mixture was cooled to 0° C. A solution of(9H-fluoren-9-yl)methyl carbonochloridate (22.06 g) in dichloromethane (35 mL) was added, and the reaction mixture was stirred for 2 hours. The reaction mixture was concentrated, and the residue was purified by silica gel chromatography, eluting with petroleum ether in ethyl acetate (10%-25%) to provide the title compound. MS (ESI) m/e 314 (M+Na)⁺.

### 2.58.2 (2S,3S,4S,5R,6S)-methyl 6-(5-(4-(((9H-fluoren-9-yl)methoxy)carbonylamino)but-1-ynyl)-2-formylphenoxy)-3,4,5-triacetoxy-tetrahydro-2H-pyran-2-carboxylate

Example 2.58.3 (2.7 g), Example 2.58.1 (2.091 g), bis(triphenylphosphine)palladium(II) chloride (0.336 g) and copper(1) iodide (0.091 g) were weighed into a vial and flushed with a stream of nitrogen. Triethylamine (2.001 mL) and tetrahydrofuran (45 mL) were added, and the reaction was stirred at room temperature. After stirring for 16 hours, the reaction mixture was diluted with ethyl acetate (200 mL) and washed with water (100 mL) and brine (100 mL). The organic layer was dried over magnesium sulfate, filtered, and concentrated. The residue was purified by silica gel chromatography, eluting with petroleum ether in ethyl acetate (10%-50%), to provide the title compound. MS (ESI) m/e 750 (M+Na)⁺.

### 2.58.3 (2S,3S,4S,5R,6S)-methyl 6-(5-(4-(((9H-fluoren-9-yl)methoxy)carbonylamino)butyl)-2-formylphenoxy)-3,4,5-triacetoxy-tetrahydro-2H-pyran-2-carboxylate

Example 2.58.2 (1.5 g) and tetrahydrofuran (45 mL) were added to 10% Pd—C (0.483 g) in a 100 mL pressure bottle and the mixture was stirred for 16 hours under 1 atm H₂ at room temperature. The reaction mixture was filtered and concentrated to provide the title compound. MS (ESI) m/e 754 (M+Na)⁺.

### 2.58.4 (2S,3S,4S,5R,6S)-methyl 6-(5-(4-(((9H-fluoren-9-yl)methoxy)carbonylamino)butyl)-2-(hydroxymethyl)phenoxy)-3,4,5-triacetoxy-tetrahydro-2H-pyran-2-carboxylate

A solution of Example 2.58.3 (2.0 g) in tetrahydrofuran (7.00 mL) and methanol (7 mL) was cooled to 0° C. and NaBH₄ (0.052 g) was added in one portion. After 30 minutes, the reaction mixture was diluted with ethyl acetate (150 mL) and water (100 mL). The organic layer was separated, washed with brine (100 mL), dried over magnesium sulfate, filtered, and concentrated. The residue was purified by silica gel chromatography, eluting with petroleum ether in ethyl acetate (10-40%), to provide the title compound. MS (ESI) m/e 756 (M+Na)⁺.

### 2.58.5 (2S,3S,4S,5R,6S)-methyl 6-(5-(4-(((9H-fluoren-9-yl)methoxy)carbonylamino)butyl)-2-(((4-nitrophenoxy)carbonyloxy)methyl)phenoxy)-3,4,5-triacetoxy-tetrahydro-2H-pyran-2-carboxylate

To a solution of Example 2.58.4 (3.0 g) and bis(4-nitrophenyl) carbonate (2.488 g) in dry acetonitrile (70 mL) at 0° C. was added N,N-diisopropylethylamine (1.07 mL). After stirring at room temperature for 16 hours, the reaction mixture was concentrated to give a residue, which was purified by silica gel chromatography, eluting with petroleum ether in ethyl acetate (10%-50%), to provide the title compound. MS (ESI) m/e 921 (M+Na)⁺.

### 2.58.6 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(2-(N,N-dimethylsulfamoyl)ethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.58.5 (40.8 mg) and Example 1.36 (40 mg) in N,N-dimethylformamide (4 mL) was added N,N-diisopropylethylamine (0.026 mL). The reaction mixture was slowly warmed to room temperature and stirred overnight. To the reaction mixture was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1278.7 (M−H)⁻.

### 2.58.7 2-{6-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]-2-methyl-3,3-dioxido-7-oxo-8-oxa-3lambda⁶-thia-2,6-diazanonan-9-yl}-5-(4-{([(2,5-dioxo-2,5-dihydro-1H-pyrrol-1-yl)acetyl]amino}butyl)phenyl beta-D-glucopyranosiduronic acid

To a solution of Example 2.58.6 (35.1 mg) in N,N-dimethylformamide (4 mL) was added 2,5-dioxopyrrolidin-1-yl 2-(2,5-dioxo-2,5-dihydro-1H-pyrrol-1-yl)acetate (6.93 mg) and N,N-diisopropylethylamine (0.026 mL). The reaction mixture was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. ¹H NMR (400 MHz, dimethyl sulfoxide-d₆) δ ppm 12.85 (s, 1H), 8.02 (dd, 2H), 7.76 (d, 1H), 7.58 (d, 1H), 7.53-7.37 (m, 3H), 7.32 (td, 2H), 7.24 (s, 1H), 7.16 (dd, 1H), 7.04 (s, 2H), 6.99-6.87 (m, 2H), 6.81 (d, 1H), 5.08 (d, 2H), 4.99 (d, 1H), 4.92 (s, 2H), 3.95 (s, 2H), 3.86 (q, 3H), 3.47-3.14 (m, 9H), 2.99 (dt, 4H), 2.72 (s, 3H), 2.60 (s, 3H), 2.06 (s, 3H), 1.49 (p, 2H), 1.41-1.27 (m, 4H), 1.29-0.86 (m, 10H), 0.80 (d, 7H). MS (ESI) m/e 1413.4 (M−H)⁻.

### 2.59 Synthesis of 6-[8-(1,3-benzothiazol-2-ylcarbamoyl)-3,4-dihydroisoquinolin-2(1H)-yl]-3-(1-{[3-(2-{({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(dimethylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid

### 2.59.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(3-(dimethylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.38. MS (ESI) m/e 1243.7 (M+H)⁺.

### 2.59.2 6-[8-(1,3-benzothiazol-2-ylcarbamoyl)-3,4-dihydroisoquinolin-2(1H)-yl]-3-(1-{[3-(2-{({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(dimethylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid

The title compound was prepared as described in Example 2.58.7, substituting Example 2.58.6 with Example 2.59.1. ¹H NMR (400 MHz, dimethyl sulfoxide-d₆) δ ppm 8.02 (dd, 2H), 7.76 (d, 1H), 7.58 (d, 1H), 7.44 (ddd, 3H), 7.32 (td, 2H), 7.24 (s, 1H), 7.13 (dd, 1H), 7.04 (s, 2H), 6.99-6.86 (m, 2H), 6.81 (d, 1H), 5.06 (d, 2H), 4.98 (d, 1H), 4.92 (s, 2H), 3.95 (s, 2H), 3.85 (q, 3H), 3.77 (d, 2H), 3.39 (q, 5H), 3.27 (q, 4H), 2.99 (dt, 4H), 2.88 (s, 2H), 2.81-2.66 (m, 5H), 2.06 (d, 3H), 1.50 (p, 2H), 1.34 (dd, 4H), 1.27-0.85 (m, 9H), 0.79 (d, 6H). MS (ESI) m/e 1401.3 (M+H)⁺.

### 2.60 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfamoylethyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid

### 2.60.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(2-sulfamoylethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.18.20. MS (ESI) m/e 1251.2 (M+H)⁺.

### 2.60.2 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfamoylethyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid

The title compound was prepared as described in Example 2.58.7, substituting Example 2.58.6 with Example 2.60.1. ¹H NMR (400 MHz, dimethyl sulfoxide-d₆) δ ppm 12.84 (s, 2H), 8.04 (dd, 2H), 7.77 (d, 1H), 7.60 (d, 1H), 7.53-7.38 (m, 3H), 7.38-7.30 (m, 2H), 7.26 (s, 1H), 7.16 (d, 1H), 7.05 (s, 2H), 6.96-6.77 (m, 5H), 5.09 (s, 2H), 5.00 (d, 1H), 4.94 (s, 2H), 3.97 (s, 2H), 3.87 (q, 3H), 3.48-3.16 (m, 5H), 3.09-2.94 (m, 4H), 2.07 (s, 3H), 1.50 (d, 2H), 1.36 (d, 3H), 1.29-0.88 (m, 9H), 0.81 (d, 7H). MS (ESI) m/e 1385.5 (M−H)⁻.

### 2.61 Synthesis of 6-[8-(1,3-benzothiazol-2-ylcarbamoyl)-3,4-dihydroisoquinolin-2(1H)-yl]-3-(1-{[3-(2-{({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(methylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid

### 2.61.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(3-(methylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.39. MS (ESI) m/e 1228.8 (M+H)⁺.

### 2.61.2 6-[8-(1,3-benzothiazol-2-ylcarbamoyl)-3,4-dihydroisoquinolin-2(1H)-yl]-3-(1-{[3-(2-{[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(methylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid

The title compound was prepared as described in Example 2.58.7, substituting Example 2.58.6 with Example 2.61.1. ¹H NMR (501 MHz, dimethyl sulfoxide-d₆) δ ppm 12.83 (s, 1H), 8.06 (s, 1H), 8.01 (dd, 1H), 7.77 (d, 1H), 7.71 (d, 0H), 7.60 (d, 1H), 7.45 (tdd, 3H), 7.38-7.29 (m, 2H), 7.26 (s, 1H), 7.15 (d, 1H), 7.05 (d, 1H), 6.96-6.90 (m, 2H), 6.82 (d, 1H), 5.07 (s, 2H), 5.01 (t, 1H), 4.94 (s, 2H), 3.97 (s, 2H), 3.87 (q, 3H), 3.79 (d, 2H), 3.28 (p, 2H), 3.09-2.93 (m, 3H), 2.52 (d, 3H), 2.35-2.26 (m, 2H), 2.07 (d, 2H), 1.60-1.44 (m, 2H), 1.34 (d, 3H), 1.29-0.88 (m, 6H), 0.81 (d, 5H). MS (ESI) m/e 1363.5 (M−H)⁻.

### 2.62 Synthesis of 3-{1-[(3-{2-[(3-amino-3-oxopropyl)({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid

2.62.1 3-(1-((3-(2-((3-amino-3-oxopropyl)(((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.32.2. MS (ESI) m/e 1214.6 (M+H)⁺.

### 2.62.2 3-{1-[(3-{2-[(3-amino-3-oxopropyl)({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid

The title compound was prepared as described in Example 2.58.7, substituting Example 2.58.6 with Example 2.62.1. ¹H NMR (501 MHz, dimethyl sulfoxide-d₆) δ ppm 12.83 (s, 2H), 8.06 (s, 1H), 8.01 (d, 1H), 7.77 (d, 1H), 7.60 (d, 1H), 7.53-7.38 (m, 3H), 7.34 (q, 2H), 7.26 (s, 1H), 7.15 (d, 1H), 7.05 (s, 2H), 6.93 (d, 2H), 6.87-6.73 (m, 2H), 5.07 (d, 2H), 5.04-4.97 (m, 1H), 4.94 (s, 2H), 3.97 (s, 2H), 3.87 (q, 3H), 3.79 (d, 2H), 3.29 (t, 3H), 3.10-2.95 (m, 4H), 2.32 (p, 2H), 2.07 (d, 3H), 1.51 (dd, 2H), 1.36 (dd, 5H), 1.30-0.86 (m, 8H), 0.81 (d, 6H). MS (ESI) m/e 1349.5 (M−H)⁻.

### 2.63 Synthesis of 2-[({[2-({3-[(4-{6-[3-(1,3-benzothiazol-2-ylcarbamoyl)-1H-indol-5-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid

### 2.63.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(3-(benzo[d]thiazol-2-ylcarbamoyl)-1H-indol-5-yl)picolinic acid

The title compound was prepared by substituting Example 1.34.5 for Example 1.12.10 and Example 2.58.5 for Example 2.11.6 in Example 2.11.7.

### 2.63.2 2-[({[2-({3-[(4-{6-[3-(1,3-benzothiazol-2-ylcarbamoyl)-1H-indol-5-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid

The title compound was prepared by substituting Example 2.63.1 for Example 2.9.1 in Example 2.10. ¹H NMR (400 MHz, dimethyl sulfoxide-d₆) δ ppm 12.47 (bs, 1H), 12.16 (d, 1H), 9.01 (s, 1H), 8.69 (d, 1H), 8.11-8.04 (m, 4H), 7.99 (d, 1H), 7.76 (d, 1H), 7.64 (d, 1H), 7.48 (s, 1H), 7.45 (t, 1H), 7.31 (t, 1H), 7.19 (t, 1H), 7.07 (s, 1H), 6.94 (s, 1H), 6.86 (d, 1H), 5.10 (s, 2H), 5.03 (d, 1H), 3.99 (s, 2H), 3.90 (m, 3H), 3.48 (m, 3H), 3.28 (m, 2H), 3.05 (m, 4H), 2.93 (s, 2H), 2.88 (s, 2H), 2.54-2.53 (m, 2H), 2.24 (s, 3H), 1.54 (m, 2H), 1.40 (m, 4H), 1.30-1.22 (m, 6H), 1.20-1.14 (m, 6H), 1.11-0.96 (m, 2H), 0.87 (d, 6H). MS (ESI) m/e 1300 (M+Na)⁺, 1276 (M−H)⁻.

### 2.64 Synthesis of 2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-5-(4-{([(2,5-dioxo-2,5-dihydro-1H-pyrrol-1-yl)acetyl]amino}butyl)phenyl beta-D-glucopyranosiduronic acid

### 2.64.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(1-(benzo[d]thiazol-2-ylcarbamoyl)-5,6-dihydroimidazo[1,5-a]pyrazin-7(8H)-yl)picolinic acid

The title compound was prepared by substituting Example 1.4.10 for Example 1.12.10 and Example 2.58.5 for Example 2.11.6 in Example 2.11.7. MS (ESI) m/e 1133 (M+H)⁺, 1131 (M−H)⁻.

### 2.64.2 2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-5-(4-{[(2,5-dioxo-2,5-dihydro-1H-pyrrol-1-yl)acetyl]amino}butyl)phenyl beta-D-glucopyranosiduronic acid

The title compound was prepared by substituting Example 2.64.1 for Example 2.9.1 in Example 2.10. ¹H NMR (400 MHz, dimethyl sulfoxide-d) δ ppm 8.08 (t, 1H), 8.01 (s, 1H), 7.99 (d, 1H), 7.76 (d, 1H), 7.61 (d, 1H), 7.46 (t, 1H), 7.34 (s, 1H), 7.33 (t, 1H), 7.17 (m, 3H), 7.08 (s, 2H), 6.92 (s, 1H), 6.84 (d, 1H), 5.12 (s, 2H), 5.05 (s, 2H), 5.02 (d, 1H), 4.27 (m, 2H), 4.10 (m, 2H), 3.99 (s, 2H), 3.91 (m, 2H), 3.84 (s, 2H), 3.70 (m, 2H), 3.42 (t, 2H), 3.35 (t, 2H), 3.30 (t, 2H), 3.06 (m, 5H), 2.53 (m, 2H), 2.14 (s, 3H), 1.53 (m, 2H), 1.43-1.35 (m, 4H), 1.27 (m, 4H), 1.14 (q, 4H), 1.03 (dd, 2H), 0.86 (s, 6H). MS (ESI) m/e 1270 (M+H)⁺, 1268 (M−H)⁻.

### 2.65 Synthesis of (6S)-2,6-anhydro-6-(2-{2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-5-({N-[(2,5-dioxo-2,5-dihydro-1H-pyrrol-1-yl)acetyl]-L-valyl-L-alanyl}amino)phenyl}ethyl)-L-gulonic acid

### 2.65.1 (3R,4S,5R,6R)-3,4,5-tris(benzyloxy)-6-(benzyloxymethyl)-tetrahydropyran-2-one

To a solution of (3R,4S,5R,6R)-3,4,5-tris(benzyloxy)-6-((benzyloxy)methyl)tetrahydro-2H-pyran-2-ol (75 g) in dimethyl sulfoxide (400 mL) at 0° C. was added acetic anhydride (225 mL). The mixture was stirred for 16 hours at room temperature before cooled to 0° C. A large volume of water was added, and the stirring was stopped and the reaction mixture was allowed to settle for 3 hours (the crude lactone was at the bottom of the flask). The supernatant was removed, and the crude mixture was diluted with ethyl acetate, washed 3 times with water, neutralized with saturated aqueous solution of NaHCO₃, and washed again twice with water. The organic layer was then dried over magnesium sulfate, filtered and concentrated to provide the title compound. MS (ESI) m/e 561 (M+Na)⁺.

### 2.65.2 (3R,4S,5R,6R)-3,4,5-tris(benzyloxy)-6-(benzyloxymethyl)-2-ethynyl-tetrahydro-2H-pyran-2-ol

To a solution of ethynyltrimethylsilane (18.23 g) in tetrahydrofuran (400 mL) under nitrogen and chilled in a dry ice/acetone bath (internal temp −65° C.) was added 2.5M BuLi in hexane (55.7 mL) dropwise, keeping the temperature below −60° C. The mixture was stirred in a cold bath for 40 minutes, followed by an ice-water bath (internal temp rose to 0.4° C.) for 40 minutes, and finally cooled to −75° C. again. A solution of Example 2.55.1 (50 g) in tetrahydrofuran (50 mL) was added dropwise, keeping the internal temperature below −70° C. The mixture was stirred in a dry ice/acetone bath for an additional 3 hours. The reaction mixture was quenched with saturated aqueous NaHCO₃ solution (250 mL). The mixture was allowed to warm to room temperature, extracted with ethyl acetate (3×300 mL), dried over MgSO₄, filtered, and concentrated in vacuo to provide the title compound. MS (ESI) m/e 659 (M+Na)⁺.

### 2.65.3 trimethyl(((3S,4R,5R,6R)-3,4,5-tris(benzyloxy)-6-(benzyloxymethyl)-tetrahydro-2H-pyran-2-yl)ethynyl)silane

To a mixture of Example 2.65.2 (60 g) in acetonitrile (450 mL) and dichloromethane (150 mL) at −15° C. in an ice-salt bath was added triethylsilane (81 mL) dropwise, followed by addition of boron trifluoride diethyl ether complex (40.6 mL) at such a rate that the internal temperature did not exceed −10° C. The mixture was stirred between −15° C. and −10° C. for 2 hours. The reaction mixture was quenched with saturated aqueous NaHCO₃ solution (275 mL) and stirred for 1 hour at room temperature. The mixture was extracted with ethyl acetate (3×550 mL). The combined extracts were dried over MgSO₄, filtered, and concentrated. The residue was purified by flash chromatography eluting with a gradient of 0% to 7% ethyl acetate/petroleum ether to provide the title compound. MS (ESI) m/e 643 (M+Na)⁺.

### 2.65.4 (2R,3R,4R,5S)-3,4,5-tris(benzyloxy)-2-(benzyloxymethyl)-6-ethynyl-tetrahydro-2H-pyran

To a mixed solution of Example 2.65.3 (80 g) in dichloromethane (200 mL) and methanol (1000 mL) was added 1N aqueous NaOH solution (258 mL). The mixture was stirred at room temperature for 2 hours. The solvent was removed. The residue was then partitioned between water and dichloromethane. The extracts were washed with brine, dried over Na₂SO₄, filtered, and concentrated to provide the title compound. MS (ESI) m/e 571 (M+Na)⁺.

### 2.65.5 (2R,3R,4R,5S)-2-(acetoxymethyl)-6-ethynyl-tetrahydro-2H-pyran-3,4,5-triyl triacetate

To a solution of Example 2.65.4 (66 g) in acetic anhydride (500 mL) cooled by an ice/water bath was added boron trifluoride diethyl ether complex (152 mL) dropwise. The mixture was stirred at room temperature for 16 hours, cooled with an ice/water bath and neutralized with saturated aqueous NaHCO₃ solution. The mixture was extracted with ethyl acetate (3×500 mL), dried over Na₂SO₄ and concentrated in vacuo. The residue was purified by flash chromatography eluting with a gradient of 0% to 30% ethyl acetate/petroleum ether to provide the title compound. MS (ESI) m/e 357 (M+H)⁺.

### 2.65.6 (3R,4R,5S,6R)-2-ethynyl-6-(hydroxymethyl)-tetrahydro-2H-pyran-3,4,5-triol

To a solution of Example 2.65.5 (25 g) in methanol (440 mL) was added sodium methanolate (2.1 g). The mixture was stirred at room temperature for 2 hours, then neutralized with 4M HCl in dioxane. The solvent was removed, and the residue was adsorbed onto silica gel and loaded onto a silica gel column. The column was eluted with a gradient of 0 to 100% ethyl acetate/petroleum ether then 0% to 12% methanol/ethyl acetate to provide the title compound. MS (ESI) m/e 211 (M+Na)⁺.

### 2.65.7 (2S,3S,4R,5R)-6-ethynyl-3,4,5-trihydroxy-tetrahydro-2H-pyran-2-carboxylic acid

A three-necked round bottom flask was charged with Example 2.65.6 (6.00 g). KBr (0.30 g), tetrabutylammonium bromide (0.41 g) and 60 mL of saturated aqueous NaHCO₃ solution. (2,2,6,6-Tetramethylpiperidin-1-yl)oxidanyl (0.15 g) in 60 mL dichloromethane was added. The mixture was stirred vigorously and cooled in an ice-salt bath to −2° C. internal temperature. A solution of brine (12 mL), aqueous NaHCO₃ solution (24 mL) and NaOCl (154 mL) was added dropwise such that the internal temperature was maintained below 2° C. The pH of the reaction mixture was maintained in the 8.2-8.4 range with the addition of solid Na₂CO₃. After a total of 6 hours the reaction was cooled to 3° C. internal temperature and ethanol (˜20 mL) was added dropwise and was stirred for ˜30 minutes. The mixture was transferred to a separatory funnel, and the dichloromethane layer was discarded. The pH of the aqueous layer was adjusted to 2-3 using 1 M aqueous HCl. The aqueous layer was then concentrated to dryness. Methanol (100 mL was) added to the dry solid, and the slurry was stirred for ˜30 minutes. The mixture was filtered over a pad of diatomaceous earth, and the residue in the funnel was washed with ˜100 mL of methanol. The filtrate was concentrated under reduced pressure to obtain the title compound.

### 2.65.8 (2S,3S,4R,5R)-methyl 6-ethynyl-3,4,5-trihydroxytetrahydro-2H-pyran-2-carboxylate

A 500 mL three-necked round bottom flask was charged with a suspension of Example 2.65.7 (6.45 g) in methanol (96 mL) and was cooled in an ice-salt-bath with internal temperature of −1° C. Neat thionyl chloride (2.79 mL) was carefully added. The internal temperature kept rising throughout the addition but did not exceed 10° C. The reaction was allowed to slowly warm up to 15-20° C. over 2.5 hours. After 2.5 hours, the reaction was concentrated to provide the title compound.

### 2.65.9 (3S,4R,5S,6S)-2-ethynyl-6-(methoxycarbonyl)tetrahydro-2H-pyran-3,4,5-triyl triacetate

Example 2.65.8 (6.9 g) as a solution in N,N-dimethylformamide (75 mL) was added 4-dimethylaminopyridine (0.17 g) and acetic anhydride (36.1 mL). The suspension was cooled in an ice-bath and pyridine (18.04 mL) was added via syringe over 15 minutes. The reaction was allowed to warm to room temperature overnight. Additional acetic anhydride (12 mL) and pyridine (6 mL) were added and stirring was continued for an additional 6 hours. The reaction was cooled in an ice-bath and 250 mL of saturated aqueous NaHCO₃ solution was added and stirred for 1 hour. Water (100 mL) was added, and the mixture was extracted with ethyl acetate. The organic extract was washed twice with saturated CuSO₄ solution, dried and concentrated. The residue was purified by flash chromatography, eluting with 50% ethyl acetate/petroleum ether to provide the title compound. ¹H NMR (500 MHz, methanol-d₄) δ ppm 5.29 (t, 1H), 5.08 (td, 2H), 4.48 (dd, 1H), 4.23 (d, 1H), 3.71 (s, 3H), 3.04 (d, 1H), 2.03 (s, 3H), 1.99 (s, 3H), 1.98 (s, 4H). MS (ESI) m/e 359.9 (M+NH₄)⁺.

### 2.65.10 2-iodo-4-nitrobenzoic acid

A 3 L fully jacketed flask equipped with a mechanical stirrer, temperature probe and an addition funnel under a nitrogen atmosphere, was charged with 2-amino-4-nitrobenzoic acid (69.1 g, Combi-Blocks) and sulfuric acid, 1.5 M aqueous (696 mL). The resulting suspension was cooled to 0° C. internal temperature, and a solution of sodium nitrite (28.8 g) in water (250 mL) was added dropwise over 43 minutes with the temperature kept below 1° C. The reaction mixture was stirred at ca. 0° C. for 1 hour. A solution of potassium iodide (107 g) in water (250 mL) was added dropwise over 44 minutes with the internal temperature kept below 1° C. (Initially addition was exothermic and there was gas evolution). The reaction mixture was stirred 1 hour at 0° C. The temperature was raised to 20° C. and then stirred at ambient temperature overnight. The reaction mixture became a suspension. The reaction mixture was filtered, and the collected solid was washed with water. The wet solid (˜108 g) was stirred in 10% sodium sulfite (350 ml, with ˜200 mL water used to wash in the solid) for 30 minutes. The suspension was acidified with concentrated hydrochloric acid (35 mL), and the solid was collected by filtration and washed with water. The solid was slurried in water (1 L) and re-filtered, and the solid was left to dry in the funnel overnight. The solid was then dried in a vacuum oven for 2 hours at 60° C. The resulting solid was triturated with dichloromethane (500 mL), and the suspension was filtered and washed with additional dichloromethane. The solid was air-dried to provide the title compound. MS (ESI) m/e 291.8 (M−H)⁻.

### 2.65.11 (2-iodo-4-nitrophenyl)methanol

A flame-dried 3 L 3-necked flask was charged with Example 2.65.10 (51.9 g) and tetrahydrofuran (700 mL). The solution was cooled in an ice bath to 0.5° C., and borane-tetrahydrofuran complex (443 mL, 1M in THF) was added dropwise (gas evolution) over 50 minutes, reaching a final internal temperature of 1.3° C. The reaction mixture was stirred for 15 minutes, and the ice bath was removed. The reaction left to come to ambient temperature over 30 minutes. A heating mantle was installed, and the reaction was heated to an internal temperature of 65.5° C. for 3 hours, and then allowed to cool to room temperature while stirring overnight. The reaction mixture was cooled in an ice bath to 0° C. and quenched by dropwise addition of methanol (400 mL). After a brief incubation period, the temperature rose quickly to 2.5° C. with gas evolution. After the first 100 mL are added over ˜30 minutes, the addition was no longer exothermic, and the gas evolution ceased. The ice bath was removed, and the mixture was stirred at ambient temperature under nitrogen overnight. The mixture was concentrated to a solid, dissolved in dichloromethane/methanol and adsorbed on to silica gel (˜150 g). The residue was loaded on a plug of silica gel (3000 mL) and eluted with dichloromethane to provide the title compound. MS (DCI) m/e 296.8 (M+NH₄)⁺.

### 2.65.12 (4-amino-2-iodophenyl)methanol

A 5 L flask equipped with a mechanical stirrer, heating mantle controlled by a JKEM temperature probe and condenser was charged with Example 2.65.11 (98.83 g) and ethanol (2 L). The reaction was stirred rapidly, and iron (99 g) was added, followed by a solution of ammonium chloride (20.84 g) in water (500 mL). The reaction was heated over the course of 20 minutes to an internal temperature of 80.3° C., when it began to reflux vigorously. The mantle was dropped until the reflux calmed. Thereafter, the mixture was heated to 80° C. for 1.5 hour. The reaction was filtered hot through a membrane filter, and the iron residue was washed with hot 50% ethyl acetate/methanol (800 mL). The eluent was passed through a diatomaceous earth pad, and the filtrate was concentrated. The residue was partitioned between 50% brine (1500 mL) and ethyl acetate (1500 mL). The layers were separated, and the aqueous layer was extracted with ethyl acetate (400 mL×3). The combined organic layers were dried over sodium sulfate, filtered and concentrated to provide the title compound, which was used without further purification. MS (DCI) m/e 266.9 (M+NH₄)⁺.

### 2.65.13 4-(((tert-butyldimethylsilyl)oxy)methyl)-3-iodoaniline

A 5 L flask with a mechanical stirrer was charged with Example 2.65.12 (88 g) and dichloromethane (2 L). The suspension was cooled in an ice bath to an internal temperature of 2.5° C. and tert-butylchlorodimethylsilane (53.3 g) was added portion-wise over 8 minutes. After 10 minutes, 1H-imidazole (33.7 g) was added portionwise to the cold reaction. The reaction was stirred 90 minutes while the internal temperature rose to 15° C. The reaction mixture was diluted with water (3 L) and dichloromethane (1 L). The layers were separated, and the organic layer was dried over sodium sulfate, filtered, and concentrated to an oil. The residue was purified by silica gel chromatography (1600 g silica gel), eluting a gradient of 0-25% ethyl acetate in heptane, to provide the title compound.

### 2.65.14 (S)-2-((S)-2-((((9H-fluoren-9-yl)methoxy)carbonyl)amino)-3-methylbutanamido)propanoic acid

To a solution of (S)-2-((((9H-fluoren-9-yl)methoxy)carbonyl)amino)-3-methylbutanoic acid (6.5 g) in dimethoxyethane (40 mL) was added (S)-2-aminopropanoic acid (1.393 g) and sodium bicarbonate (1.314 g) in water (40 mL). Tetrahydrofuran (20 mL) was added to aid solubility. The resulting mixture was stirred at room temperature for 16 hours. Aqueous citric acid (15%, 75 mL) was added, and the mixture was extracted with 10% 2-propanol in ethyl acetate (2×100 mL). A precipitate formed in the organic layer. The combined organic layers were washed with water (2×150 mL). The organic layer was concentrated under reduced pressure and then triturated with diethyl ether (80 mL). After brief sonication, the title compound was collected by filtration. MS (ESI) m/e 411 (M+H)⁺.

### 2.65.15 (9H-fluoren-9-yl)methyl ((S)-1-(((S)-1-((4-(((tert-butyldimethylsilyl)oxy)methyl)-3-iodophenyl)amino)-1-oxopropan-2-yl)amino)-3-methyl-1-oxobutan-2-yl)carbamate

A solution of Example 2.65.13 (5.44 g) and Example 2.65.14 (6.15 g) in a mixture of dichloromethane (70 mL) and methanol (35.0 mL) was added ethyl 2-ethoxyquinoline-1(2H)-carboxylate (4.08 g), and the reaction was stirred overnight. The reaction mixture was concentrated and the residue was loaded onto silica gel, eluting with a gradient of 10% to 95% heptane in ethyl acetate followed by 5% methanol in dichloromethane. The product-containing fractions were concentrated, dissolved in 0.2% methanol in dichloromethane (50 mL), loaded onto silica gel and eluted with a gradient of 0.2% to 2% methanol in dichloromethane. The product containing fractions were collected to provide the title compound. MS (ESI) m/e 756.0 (M+H)⁺.

### 2.65.16 (2S,3S,4R,5S,6S)-2-((5-((S)-2-((S)-2-((((9H-fluoren-9-yl)methoxy)carbonyl)amino)-3-methylbutanamido)propanamido)-2-(((tert-butyldimethylsilyl)oxy)methyl)phenyl)ethynyl)-6-(methoxycarbonyl)tetrahydro-2H-pyran-3,4,5-triyl triacetate

A solution of Example 2.65.9 (4.500 g), Example 2.65.15 (6.62 g), copper(I) iodide (0.083 g) and bis(triphenylphosphsine)palladium(II) dichloride (0.308 g) were combined in vial and degassed. N,N-dimethylformamide (45 mL) and N-ethyl-N-isopropylpropan-2-amine (4.55 mL) were added, and the reaction vessel was flushed with nitrogen and stirred at room temperature overnight. The reaction was partitioned between water (100 mL) and ethyl acetate (250 mL). The layers were separated, and the organic layer was dried over magnesium sulfate, filtered, and concentrated. The residue was purified by silica gel chromatography, eluting with a gradient of 5% to 95% ethyl acetate in heptane. The product containing fractions were collected, concentrated and purified by silica gel chromatography, eluting with a gradient of 0.25% to 2.5% methanol in dichloromethane to provide the title compound. MS (ESI) m/e 970.4 (M+H)⁺.

### 2.65.17 (2S,3S,4R,5S,6S)-2-(5-((S)-2-((S)-2-((((9H-fluoren-9-yl)methoxy)carbonyl)amino)-3-methylbutanamido)propanamido)-2-(((tert-butyldimethylsilyl)oxy)methyl)phenethyl)-6-(methoxycarbonyl)tetrahydro-2H-pyran-3,4,5-triyl triacetate

Example 2.65.16 (4.7 g) and tetrahydrofuran (95 mL) were added to 5% Pt/C (2.42 g, wet) in a 50 mL pressure bottle and the reaction was shaken for 90 minutes at room temperature under 50 psi of hydrogen. The reaction mixture was filtered and concentrated to provide the title compound. MS (ESI) m/e 974.6 (M+H)⁺.

### 2.65.18 (2S,3S,4R,5S,6S)-2-(5-((S)-2-((S)-2-((((9H-fluoren-9-yl)methoxy)carbonyl)amino)-3-methylbutanamido)propanamido)-2-(hydroxymethyl)phenethyl)-6-(methoxycarbonyl)tetrahydro-2H-pyran-3,4,5-triyl triacetate

A solution of Example 2.65.17 (5.4 g) in tetrahydrofuran (7 mL), water (7 mL) and glacial acetic acid (21 mL) was stirred overnight at room temperature. The reaction mixture was diluted with ethyl acetate (200 mL) and was washed with water (100 mL), saturated aqueous NaHCO₃ solution (100 mL), and brine (100 mL), dried over magnesium sulfate, filtered, and concentrated. The residue was purified by silica gel chromatography, eluting with a gradient of 0.5% to 5% methanol in dichloromethane, to provide the title compound. MS (ESI) m/e 860.4 (M+H)⁺.

### 2.65.19 (2S,3S,4R,5S,6S)-2-(5-((S)-2-((S)-2-((((9H-fluoren-9-yl)methoxy)carbonyl)amino)-3-methylbutanamido)propanamido)-2-((((4-nitrophenoxy)carbonyl)oxy)methyl)phenethyl)-6-(methoxycarbonyl)tetrahydro-2H-pyran-3,4,5-triyl triacetate

To a solution of Example 2.65.18 (4.00 g) and bis(4-nitrophenyl) carbonate (2.83 g) in acetonitrile (80 mL) was added N-ethyl-N-isopropylpropan-2-amine (1.22 mL) at room temperature. After stirring overnight, the reaction mixture was concentrated, dissolved in dichloromethane (250 mL) and washed with saturated aqueous NaHCO₃ solution (4×150 mL). The organic layer was dried over magnesium sulfate, filtered, and concentrated. The resulting foam was purified by silica gel chromatography, eluting with a gradient of 5% to 75% ethyl acetate in hexanes to provide the title compound. MS (ESI) m/e 1025.5 (M+H)⁺.

### 2.65.20 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)amino)ethoxy)-5,7-dimethyladamantan-1-yl)methyl)-5-methyl-1H-pyrazol-4-yl)-6-(1-(benzo[d]thiazol-2-ylcarbamoyl)-5,6-dihydroimidazo[1,5-a]pyrazin-7(8H)-yl)picolinic acid

The title compound was prepared by substituting Example 1.4.10 for Example 1.12.10 and Example 2.65.19 for Example 2.11.6 in Example 2.11.7. MS (ESI) m/e 1257 (M−H)⁻.

### 2.65.21 (6S)-2,6-anhydro-6-(2-{2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-5-({N-[(2,5-dioxo-2,5-dihydro-1H-pyrrol-1-yl)acetyl]-L-valyl-L-alanyl}amino)phenyl}ethyl)-L-gulonic acid

The title compound was prepared by substituting Example 2.65.20 for Example 2.9.1 in Example 2.10. ¹H NMR (400 MHz, dimethyl sulfoxide-d₆) δ ppm 9.88 (s, 1H), 8.26 (t, 2H), 8.00 (m, 2H), 7.76 (d, 1H), 7.61 (d, 1H), 7.46 (m, 2H), 7.38-7.30 (m, 3H), 7.21 (d, 1H), 7.15 (d, 1H), 7.07 (s, 2H), 7.04 (t, 1H), 5.12 (s, 2H), 4.97 (s, 2H), 4.39 (m, 1H), 4.28 (m, 2H), 4.22 (m, 2H), 4.12 (s, 2H), 4.09 (m, 2H), 3.84 (s, 2H), 3.58 (m, 4H), 3.33 (m, 4H), 3.18-3.00 (m, 4H), 2.94 (t, 2H), 2.80-2.55 (m, 2H), 2.13 (s, 3H), 2.08-1.91 (m, 2H), 1.56 (m, 1H), 1.39 (s, 2H), 1.30-1.20 (m, 6H), 1.26-0.95 (m, 6H), 0.85 (m, 12H). MS (ESI) m/e 1395 (M−H)⁻.

### 2.66 Synthesis of (6S)-2,6-anhydro-6-[2-(2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-L-alanyl]amino}phenyl)ethyl]-L-gulonic acid

### 2.66.1 (3R,7aS)-3-phenyltetrahydropyrrolo[1,2-c]oxazol-5(3H)-one

A solution of (S)-5-(hydroxymethyl)pyrrolidin-2-one (25 g), benzaldehyde (25.5 g) and para-toluensulfonic acid monohydrate (0.50 g) in toluene (300 mL) was heated to reflux using a Dean-Stark trap under a drying tube for 16 hours. The reaction was cooled to room temperature, and the solvent was decanted from the insoluble materials. The organic layer was washed with saturated aqueous sodium bicarbonate solution (2×) and brine (1×). The organic layer was dried over sodium sulfate, filtered and concentrated under reduced pressure. The residue was purified by flash chromatography on silica gel, eluting with 35/65 heptane/ethyl acetate, to provide the title compound. MS (DCI) m/e 204.0 (M+H)⁺.

### 2.66.2 (3R,6R,7aS)-6-bromo-3-phenyltetrahydropyrrolo[1,2-c]oxazol-5(3H)-one

To a cold (−77° C.) solution of Example 2.66.1 (44.6 g) in tetrahydrofuran (670 mL) was added lithium bis(trimethylsilyl)amide(1.0M in hexanes) (250 mL) dropwise over 40 minutes, keeping Tᵣₓₙ<−73° C. The reaction mixture was stirred at −77° C. for 2 hours, and bromine (12.5 mL) was added dropwise over 20 minutes, keeping Tᵣₓₙ<−64° C. The reaction mixture was stirred at −77° C. for 75 minutes and was quenched by the addition of 150 mL cold 10% aqueous sodium thiosulfate solution to the −77° C. reaction. The reaction mixture was warmed to room temperature and partitioned between half-saturated aqueous ammonium chloride solution and ethyl acetate. The layers were separated, and the organic was washed with water and brine, dried over sodium sulfate, filtered and concentrated under reduced pressure. The residue was purified by silica gel chromatography, eluting with a gradient of 80/20, 75/25, and 70/30 heptane/ethyl acetate to provide the title compound. MS (DCI) m/e 299.0 and 301.0 (M+NH₃+H)⁺.

### 2.66.3 (3R,6S,7aS)-6-bromo-3-phenyltetrahydropyrrolo[1,2-c]oxazol-5(3H)-one

The title compound was isolated as a by-product during the synthesis of Example 2.66.2. MS (DCI) m/e 299.0 and 301.0 (M+NH₃+H)⁺.

### 2.66.4 (3R,6S,7aS)-6-azido-3-phenyltetrahydropyrrolo[1,2-c]oxazol-5(3H)-one

To a solution of Example 2.66.2 (19.3 g) in N,N-dimethylformamide (100 mL) was added sodium azide (13.5 g). The reaction mixture was heated to 60° C. for 2.5 hours. The reaction mixture was cooled to room temperature and quenched by the addition of water (500 mL) and ethyl acetate (200 mL). The layers were separated, and the organic layer was washed brine. The combined aqueous layers were back-extracted with ethyl acetate (50 mL). The combined organic layers were dried with sodium sulfate, filtered and concentrated under reduced pressure. The residue was purified by silica gel chromatography, eluting with 78/22 heptane/ethyl acetate, to provide the title compound. MS (DCI) m/e 262.0 (M+NH₃+H)⁺.

### 2.66.5 (3R,6S,7aS)-6-amino-3-phenyltetrahydropyrrolo[1,2-c]oxazol-5(3H)-one

To a solution of Example 2.66.4 (13.5 g) in tetrahydrofuran (500 mL) and water (50 mL) was added polymer-supported triphenylphosphine (55 g). The reaction was mechanically stirred overnight at room temperature. The reaction mixture was filtered through diatomaceous earth, eluting with ethyl acetate and toluene. The solution was concentrated under reduced pressure, dissolved in dichloromethane (100 mL), dried with sodium sulfate, then filtered and concentrated to provide the title compound, which was used in the subsequent step without further purification. MS (DCI) m/e 219.0 (M+H)⁺.

### 2.66.6 (3R,6S,7aS)-6-(dibenzylamino)-3-phenyltetrahydropyrrolo[1,2-c]oxazol-5(3H)-one

To a solution of Example 2.66.5 (11.3 g) in N,N-dimethylformamide (100 mL) was added potassium carbonate (7.0 g), potassium iodide (4.2 g), and benzyl bromide (14.5 mL). The reaction was stirred at room temperature overnight and quenched by the addition of water and ethyl acetate. The layers were separated, and the organic layer was washed brine. The combined aqueous layers were back-extracted with ethyl acetate. The combined organic layers were dried with sodium sulfate, filtered and concentrated under reduced pressure. The residue was purified by silica gel chromatography, eluting with a gradient of 10 to 15% ethyl acetate in heptane to give a solid that was triturated with heptane to provide the title compound. MS (DCI) m/e 399.1 (M+H)⁺.

### 2.66.7 (3S,5S)-3-(dibenzylamino)-5-(hydroxymethyl)pyrrolidin-2-one

To a solution of Example 2.66.6 (13 g) in tetrahydrofuran (130 mL) was added para-toluene sulfonic acid monohydrate (12.4 g) and water (50 mL), and the reaction was heated to 65° C. for 6 days. The reaction mixture was cooled to room temperature and was quenched by the addition of saturated aqueous sodium bicarbonate and ethyl acetate. The layers were separated, and the organic layer was washed with brine. The combined aqueous layers were back-extracted with ethyl acetate. The combined organic layers were dried with sodium sulfate, filtered and concentrated under reduced pressure. The waxy solids were triturated with heptane (150 mL) to provide the title compound. MS (DCI) m/e 311.1 (M+H)⁺.

### 2.66.8 (3S,5S)-5-(((tert-butyldimethylsilyl)oxy)methyl)-3-(dibenzylamino)pyrrolidin-2-one

To a solution of Example 2.66.7 (9.3 g) and 1H-imidazole (2.2 g) in N,N-dimethylformamide was added tert-butylchlorodimethylsilane (11.2 mL, 50 weight % in toluene), and the reaction was stirred overnight. The reaction mixture was quenched by the addition of water and diethyl ether. The layers were separated, and the organic layer was washed with brine. The combined aqueous layers were back-extracted with diethyl ether. The combined organic layers were dried with sodium sulfate, filtered and concentrated under reduced pressure. The residue was purified by silica gel chromatography, eluting with 35% ethyl acetate in heptane, to provide the title compound. MS (DCI) m/e 425.1 (M+H)⁺.

### 2.66.9 tert-butyl 2-((3S,5S)-5-(((tert-butyldimethylsilyl)oxy)methyl)-3-(dibenzylamino)-2-oxopyrrolidin-1-yl)acetate

To a cold (0° C.) solution of Example 2.66.8 (4.5 g) in tetrahydrofuran (45 mL) was added 95% sodium hydride (320 mg) in two portions. The cold solution was stirred for 40 minutes, and tert-butyl 2-bromoacetate (3.2 mL) was added. The reaction mixture was warmed to room temperature and stirred overnight. The reaction mixture was quenched by the addition of water and ethyl acetate. The layers were separated, and the organic layer was washed with brine. The combined aqueous layers were back-extracted with ethyl acetate. The combined organic layers were dried with sodium sulfate, filtered and concentrated under reduced pressure. The residue was purified by silica gel chromatography, eluting with a gradient of 5-12% ethyl acetate in heptane, to provide the title compound. MS (DCI) m/e 539.2 (M+H)⁺.

### 2.66.10 tert-butyl 2-((3S,5S)-3-(dibenzylamino)-5-(hydroxymethyl)-2-oxopyrrolidin-1-yl)acetate

To a solution of Example 2.66.9 (5.3 g) in tetrahydrofuran (25 mL) was added tetrabutylammonium fluoride (11 mL, 1.0M in 95/5 tetrahydrofuran/water). The reaction mixture was stirred at room temperature for one hour and was quenched by the addition of saturated aqueous ammonium chloride solution, water and ethyl acetate. The layers were separated, and the organic layer was washed with brine. The combined aqueous layers were back-extracted with ethyl acetate. The combined organic layers were dried with sodium sulfate, filtered and concentrated under reduced pressure. The residue was purified by silica gel chromatography, eluting with 35% ethyl acetate in heptane, to provide the title compound. MS (DCI) m/e 425.1 (M+H)⁺.

### 2.66.11 tert-butyl 2-((3S,5S)-5-((2-((4-((tert-butyldiphenylsilyl)oxy)-2,2-dimethylbutoxy)sulfonyl)ethoxy)methyl)-3-(dibenzylamino)-2-oxopyrrolidin-1-yl)acetate

To a solution of Example 2.66.10 (4.7 g) in dimethyl sulfoxide (14 mL) was added a solution of 4-((tert-butyldiphenylsilyl)oxy)-2,2-dimethylbutyl ethenesulfonate (14.5 g) in dimethyl sulfoxide (14 mL). Potassium carbonate (2.6 g) and water (28 μL) were added, and the reaction was heated at 60° C. under nitrogen for one day. The reaction was cooled to room temperature, and quenched by the addition of brine solution, water and diethyl ether. The layers were separated, and the organic layer was washed with brine. The combined aqueous layers were back-extracted with diethyl ether. The combined organic layers were dried with sodium sulfate, filtered and concentrated under reduced pressure. The residue was purified by silica gel chromatography, eluting with a gradient of 15-25% ethyl acetate in heptane, to provide the title compound. MS (ESI+) m/e 871.2 (M+H)⁺.

### 2.66.12 tert-butyl 2-((3S,5S)-3-amino-5-((2-((4-((tert-butyldiphenylsilyl)oxy)-2,2-dimethylbutoxy)sulfonyl)ethoxy)methyl)-2-oxopyrrolidin-1-yl)acetate

Example 2.66.11 (873 mg) was dissolved in ethyl acetate (5 mL) and methanol (15 mL), and palladium hydroxide on carbon, 20% by wt (180 mg) was added. The reaction mixture was stirred under a hydrogen atmosphere (30 psi) at room temperature for 30 hours, then at 50° C. for one hour. The reaction was cooled to room temperature, filtered, and concentrated to give the desired product. MS (ESI+) m/e 691.0 (M+H)⁺.

### 2.66.13 4-(((3S,5S)-1-(2-(tert-butoxy)-2-oxoethyl)-5-((2-((4-((tert-butyldiphenylsilyl)oxy)-2,2-dimethylbutoxy)sulfonyl)ethoxy)methyl)-2-oxopyrrolidin-3-yl)amino)-4-oxobut-2-enoic acid

Maleic anhydride (100 mg) was dissolved in dichloromethane (0.90 mL), and a solution of Example 2.66.12 (650 mg) in dichloromethane (0.90 mL) was added dropwise, then heated at 40° C. for 2 hours. The reaction mixture was directly purified by silica gel chromatography, eluting with a gradient of 1.0-2.5% methanol in dichoromethane containing 0.2% acetic acid. After concentrating the product-bearing fractions, toluene (10 mL) was added and the mixture was concentrated again to provide the title compound. MS (ESI−) m/e 787.3 (M−H)⁻.

### 2.66.14 tert-butyl 2-((3S,5S)-5-((2-((4-((tert-butyldiphenylsilyl)oxy)-2,2-dimethylbutoxy)sulfonyl)ethoxy)methyl)-3-(2,5-dioxo-2,5-dihydro-1H-pyrrol-1-yl)-2-oxopyrrolidin-1-yl)acetate

Example 2.66.13 (560 mg) was slurried in toluene (7 mL), and triethylamine (220 μL) and sodium sulfate (525 mg) were added. The reaction mixture was heated at reflux under a nitrogen atmosphere for 6 hours, and the reaction mixture stirred at room temperature overnight. The reaction was filtered, and the solids rinsed with ethyl acetate. The eluent was concentrated under reduced pressure, and the residue was purified by silica gel chromatography, eluting with 45/55 heptane/ethyl acetate, ethyl acetate, then 97.5/2.5/0.2 dichloromethane/methanol/acetic acid to provide the title compound.

### 2.66.15 2-((3S,5S)-3-(2,5-dioxo-2,5-dihydro-1H-pyrrol-1-yl)-2-oxo-5-((2-sulfoethoxy)methyl)pyrrolidin-1-yl)acetic acid

Example 2.66.14 (1.2 g) was dissolved in trifluoroacetic acid (15 mL) and heated to 65-70° C. under nitrogen overnight. The trifluoroacetic acid was removed under reduced pressure. The residue was dissolved in acetonitrile (2.5 mL) and purified by preparative reverse-phase liquid chromatography on a Luna C18(2) AXIA column (250×50 mm, 10μ particle size) using a gradient of 5-75% acetonitrile containing 0.1% trifluoroacetic acid in water over 30 minutes, to provide the title compound. MS (ESI−) m/e 375.2 (M−H)⁻.

### 2.66.16 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (75 mg) and Example 2.65.19 (100 mg) were dissolved in N,N-dimethylformamide (0.3 mL). 1-Hydroxybenzotriazole (13 mg) and N-ethyl-N-isopropylpropan-2-amine (50 μL) were added, and the reaction was stirred at room temperature for two hours. The reaction mixture was concentrated under reduced pressure. The residue was dissolved in tetrahydrofuran and methanol (0.3 mL each), and lithium hydroxide hydrate (55 mg) in water (0.6 mL) was added. The reaction mixture was stirred at room temperature for one hour and quenched by the addition of N,N-dimethylformamide/water 1/1 (1.5 mL) with trifluoroacetic acid (0.15 mL). The solution was washed with heptane (1 mL), then purified by reverse-phase chromatography (C18 column), eluting with 20-70% acetonitrile in 0.1% trifluoroacetic acid water, to provide the title compound as a trifluoroacetic acid salt. MS (ESI−) m/e 1355.6 (M−H)⁻.

### 2.66.17 (6S)-2,6-anhydro-6-[2-(2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-L-alanyl]amino}phenyl)ethyl]-L-gulonic acid

To a solution of Example 2.66.15 (20 mg) in N,N-dimethylformamide (0.2 mL) was added O-(7-azabenzotriazol-1-yl)-N,N,N′,N′-tetramethyluronium hexafluorophosphate (20 mg) and N,N-diisopropylethylamine (18 μL). The reaction mixture was stirred for 3 minutes at room temperature and was then added to a solution of Example 2.66.16 (57 mg) and N,N-diisopropylethylamine (30 μL) in N,N-dimethylformamide (0.7 mL). The reaction mixture was stirred at room temperature for 1 hour and diluted with N,N-dimethylformamide/water 1/1 (1.0 mL). The solution was purified by reverse-phase chromatography (C18 column), eluting with 20-70% acetonitrile in 0.1% trifluoroacetic acid water, to provide the title compound. ¹H NMR (400 MHz, dimethyl sulfoxide-d₆) δ ppm 9.84 (br d, 1H), 8.18 (br d, 1H), 8.04 (m, 1H), 8.01 (d, 1H), 7.77 (dd, 2H), 7.50 (d, 1H), 7.46 (m, 3H), 7.34 (t, 1H), 7.29 (s, 1H), 7.21 (br d, 1H), 7.07 (s, 2H), 7.01 (d, 1H), 6.99 (d, 1H), 5.00 (s, 4H), 4.64 (t, 1H), 4.37 (m, 1H), 4.18 (m, 2H), 4.01 (d, 1H), 3.88 (s, 3H), 3.87 (m, 2H), 3.81 (br d, 2H), 3.73 (br m, 1H), 3.63 (m, 2H), 3.55 (m, 2H), 3.49 (m, 2H), 3.36 (br m, 6H), 3.31 (m, 2H), 3.26 (br m, 2H), 3.19 (m, 2H), 3.14 (m, 1H), 3.10 (br m, 1H), 2.94 (t, 1H), 2.81 (m, 3H), 2.74 (m, 2H), 2.60 (br m, 1H), 2.36 (m, 1H), 2.09 (s, 3H), 2.00 (m, 2H), 1.85 (m, 1H), 1.55 (br m, 1H), 1.40-0.92 (m, 14H), 0.88, 0.86, 0.83, 0.79 (d, d, s, s, total 12H). MS (ESI−) m/e 1713.7 (M−1).

### 2.67 Synthesis of 8-[2-({[(3-amino-3-oxopropyl){2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-({(2S)-2-[2-(2,5-dioxo-2,5-dihydro-1H-pyrrol-1-yl)acetamido]-3-methylbutanoyl}amino)propanoyl]amino}phenyl]-2,6-anhydro-7,8-dideoxy-L-glycero-L-gulo-octonic acid

### 2.67.1 3-(1-((3-(2-((((4-((S)-2-((S)-2-amino-3-methylbutanamido)propanamido)-2-(2-((3R,4R,5S,6S)-6-carboxy-3,4,5-trihydroxytetrahydro-2H-pyran-2-yl)ethyl)benzyl)oxy)carbonyl)(3-amino-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.65.19 (66 mg) and Example 1.32.2 (60 mg) in N,N-dimethylformamide (6 mL) was added N,N-diisopropylethylamine (0.026 mL) and 1-hydroxybenzotriazole hydrate (16.23 mg). The reaction mixture was slowly warmed to room temperature and stirred overnight. To the reaction mixture was added water (1 mL) and LiOH H₂O (20 mg). The mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1%0 trifluoroacetic acid, to provide the title compound. MS (ESI) m/e 1338.5 (M−H)⁻.

### 2.67.2 8-[2-({[(3-amino-3-oxopropyl){2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-({2S)-2-[2-(2,5-dioxo-2,5-dihydro-1H-pyrrol-1-yl)acetamido]-3-methylbutanoyl}amino)propanoyl]amino}phenyl]-2,6-anhydro-7,8-dideoxy-L-glycero-L-gulo-octonic acid

The title compound was prepared as described in Example 2.58.7, substituting Example 2.58.6 with Example 2.67.1. ¹H NMR (501 MHz, dimethyl sulfoxide-d₆) δ ppm 9.91 (d, 1H), 8.25 (dd, 2H), 8.03 (d, 1H), 7.79 (d, 1H), 7.61 (d, 6H), 7.55-7.30 (m, 7H), 7.28 (s, 1H), 7.22 (d, 1H), 7.07 (s, 2H), 6.94 (d, 1H), 6.89-6.74 (m, 1H), 5.01 (s, 3H), 4.96 (s, 2H), 4.38 (t, 1H), 4.27-4.17 (m, 1H), 4.12 (d, 2H), 3.88 (t, 2H), 3.79 (d, 1H), 3.41-3.30 (m, 3H), 3.24 (s, 2H), 3.12 (dt, 2H), 3.01 (t, 2H), 2.94 (t, 1H), 2.74 (d, 1H), 2.67-2.56 (m, 1H), 2.29 (t, 2H), 2.08 (d, 3H), 1.99 (d, 31H), 1.55 (d, 1H), 1.42-0.99 (m, 15H), 0.99-0.70 (m, 12H). MS (ESI) m/e 1477.2 (M+H)⁺.

### 2.68 Synthesis of 4-{[({2-[(3-{[4-(6-{8-[(1,3-benzothiazol-2-yl)carbamoyl]-3,4-dihydroisoquinolin-2(1H)-yl}-2-carboxypyridin-3-yl}-5-methyl-1H-pyrazol-1-yl]-methyl}-5,7-dimethyltricyclo[3.3.1.1^{3,7}]decan-1-yl)oxy]ethyl}[3-(methylamino)-3-oxopropyl]carbamoyl)oxy]methyl}-3-{3-[2-(2,5-dioxo-2,5-dihydro-1H-pyrrol-1-yl)acetamido]propoxy}phenyl beta-D-glucopyranosiduronic acid

### 2.68.1 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(3-(methylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.28.3 (38.7 mg) and Example 1.39 (39.3 mg) in N,N-dimethylformamide (6 mL) was added N,N-diisopropylethylamine (0.026 mL) and 1-hydroxybenzotriazole hydrate (6.58 mg). The reaction was slowly warmed to room temperature and stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1230.2 (M−H)⁻.

### 2.68.2 4-{[({2-[(3-{[4-(6-{8-[(1,3-benzothiazol-2-yl)carbamoyl]-3,4-dihydroisoquinolin-2(1H)-yl}-2-carboxypyridin-3-yl}-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-3-{3-[2-(2,5-dioxo-2,5-dihydro-1H-pyrrol-1-yl)acetamido]propoxy}phenyl beta-D-glucopyranosiduronic acid

The title compound was prepared as described in Example 2.58.7, substituting Example 2.58.6 with Example 2.68.1 ¹H NMR (501 MHz, dimethyl sulfoxide-d₆) δ ppm 12.88 (s, 2H), 9.93 (d, 1H), 8.36-8.22 (m, 2H), 8.04 (d, 1H), 7.80 (d, 2H), 7.76 (d, 0H), 7.62 (d, 1H), 7.56-7.42 (m, 5H), 7.41-7.33 (m, 3H), 7.28 (s, 1H), 7.22 (d, 1H), 7.08 (s, 2H), 6.95 (d, 1H), 5.01 (d, 3H), 4.96 (s, 2H), 4.39 (p, 1H), 4.22 (dd, 1H), 4.12 (d, 2H), 3.89 (t, 2H), 3.80 (d, 2H), 3.34 (t, 2H), 3.22 (d, 2H), 3.13 (dt, 2H), 3.02 (t, 2H), 2.94 (t, 1H), 2.86-2.71 (m, 1H), 2.60 (s, 2H), 2.54 (d, 4H), 2.29 (q, 2H), 2.09 (d, 3H), 2.07-1.90 (m, 3H), 1.60-1.48 (m, 1H), 1.39-1.00 (m, 17H), 0.97-0.74 (m, 15H). (ESI) m/e 1489.5 (M−H)⁻.

### 2.69 Synthesis of 2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl}-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-5-{[(2S)-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic acid

### 2.69.1 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)(3-(methylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.67.1, substituting Example 1.32.2 with Example 1.39. MS (ESI) m/e 1352.6 (M−H)⁻.

### 2.69.2 2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl}-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-5-{[(2S)-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic acid

The title compound was prepared as described in Example 2.58.7, substituting Example 2.58.6 with Example 2.67.1. ¹H NMR (501 MHz, dimethyl sulfoxide-d₆) δ ppm 12.88 (s, 2H), 9.93 (d, 1H), 8.36-8.22 (m, 2H), 8.04 (d, 1H), 7.80 (d, 2H), 7.76 (d, 0H), 7.62 (d, 1H), 7.56-7.42 (m, 5H), 7.41-7.33 (m, 3H), 7.28 (s, 1H), 7.22 (d, 1H), 7.08 (s, 2H), 6.95 (d, 1H), 5.01 (d, 3H), 4.96 (s, 2H), 4.39 (p, 1H), 4.22 (dd, 1H), 4.12 (d, 2H), 3.89 (t, 2H), 3.80 (d, 2H), 3.34 (t, 2H), 3.22 (d, 2H), 3.13 (dt, 2H), 3.02 (t, 2H), 2.94 (t, 1H), 2.86-2.71 (m, 1H), 2.60 (s, 2H), 2.54 (d, 4H), 2.29 (q, 2H), 2.09 (d, 3H), 2.07-1.90 (m, 3H), 1.60-1.48 (m, 1H), 1.39-1.00 (m, 17H), 0.97-0.74 (m, 15H). MS (ESI) m/e 1489.5 (M−H)⁻.

### 2.70 Synthesis of 2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-5-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)-3-methylbutanoyl]amino}propanoyl]amino}phenyl)-7,8-dideoxy-L-glycero-L-gulo-octonic acid

To a solution of Example 2.66.15 (17 mg) in N,N-dimethylformamide (320 μL) was added O-(7-azabenzotriazol-1-yl)-N,N,N′,N′-tetramethyluronium hexafluorophosphate (19 mg) and N,N-diisopropylethylamine (17 μL). The reaction mixture was stirred for 5 minutes and was added to a solution of Example 2.69.1 (39 mg) and N,N-diisopropylethylamine (36 μL) in N,N-dimethylformamide (320 μL). The reaction mixture was stirred for 2 hours and was diluted with N,N-dimethylformamide (2 mL). The solution was filtered and purified by reverse-phase HPLC on a Gilson system (C18 column), eluting with 20-80% acetonitrile in water containing 0.1% trifluoroacetic acid, to provide the title compound. ¹H NMR (501 MHz, dimethyl sulfoxide-d₆) δ ppm 9.82 (s, 1H), 8.15 (d, 1H), 8.00 (dd, 2H), 7.75 (d, 1H), 7.58 (d, 1H), 7.44 (ddd, 5H), 7.32 (td, 2H), 7.25 (s, 1H), 7.18 (d, 1H), 7.03 (s, 2H), 6.92 (d, 1H), 6.76 (s, 1H), 4.97 (s, 2H), 4.92 (s, 2H), 4.61 (t, 1H), 4.33 (p, 1H), 4.21-4.08 (m, 2H), 3.98 (d, 1H), 3.84 (t, 2H), 3.40-3.27 (m, 3H), 3.21 (s, 1H), 3.14-3.03 (m, 2H), 2.98 (t, 2H), 2.90 (t, 1H), 2.81-2.50 (m, 4H), 2.38-2.20 (m, 3H), 2.05 (s, 3H), 2.01-1.90 (m, 2H), 1.88-1.74 (m, 1H), 1.60-1.43 (m, 1H), 1.36-0.95 (m, 14H), 0.95-0.62 (m, 13H). MS (ESI) m/e 1710.5(M−H)⁻.

### 2.71 Synthesis of 6-{8-[(1,3-benzothiazol-2-yl)carbamoyl]-3,4-dihydroisoquinolin-2(1H)-yl}-3-[1-({3-[2-({[(4-{([(2S)-5-(carbamoylamino)-2-{[(2S)-2-{[6-(2,5-dioxo-2,5-dihydro-1H-pyrrol-1-yl)hexanoyl]amino}-3-methylbutanoyl]amino}pentanoyl]amino}phenyl)methoxy]carbonyl}amino)acetamido]-5,7-dimethyltricyclo[3.3.1.1^{3,7}]decan-1-yl}methyl)-5-methyl-1H-pyrazol-4-yl]pyridine-2-carboxylic acid

The title compound was prepared as described in Example 2.2, substituting Example 1.3.2 with Example 1.40.11. ¹H NMR (501 MHz, dimethyl sulfoxide-d₆) δ ppm 9.96 (s, 1H), 8.03 (dd, 2H), 7.78 (d, 2H), 7.59 (dd, 3H), 7.53-7.39 (m, 3H), 7.35 (q, 2H), 7.30-7.23 (m, 3H), 7.20 (d, 1H), 6.98 (s, 2H), 6.94 (d, 1H), 4.94 (d, 4H), 4.38 (t, 1H), 4.17 (dd, 1H), 3.87 (t, 2H), 3.78 (s, 2H), 3.35 (t, 2H), 3.00 (t, 3H), 2.94 (s, 0H), 2.16 (d, 1H), 2.09 (s, 3H), 1.95 (d, 1H), 1.74-1.27 (m, 10H), 1.13 (dq, 5H), 0.87-0.71 (m, 12H). MS (ESI) m/e 1355.5(M−H)⁻.

### 2.72 Synthesis of 8-[2-({[(3-amino-3-oxopropyl){2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)-3-methylbutanoyl]amino}propanoyl]amino}phenyl]-2,6-anhydro-7,8-dideoxy-L-glycero-L-gulo-octonic acid

### 2.72.1 3-(1-((3-(2-((((4-((S)-2-((S)-2-amino-3-methylbutanamido)propanamido)-2-(2-((3R,4R,5S,6S)-6-carboxy-3,4,5-trihydroxytetrahydro-2H-pyran-2-yl)ethyl)benzyl)oxy)carbonyl)(3-amino-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.65.19 (66 mg) and Example 1.32.2 (6 mL) were added N,N-diisopropylamine (0.026 mL) and 1-hydroxybenzotriazole hydrate (16.23 mg). The reaction mixture was slowly warmed to room temperature and stirred overnight. To the reaction mixture was added water (1 mL) and LiOH H₂O (20 mg), and the mixture was stirred at room temperature for 3 hours. The mixture was acidified with trifluoroacetic acid, filtered and was purified by reverse-phase HPLC on a Gilson system (C18 column), eluting with 20-80% acetonitrile in water containing 0.1% trifluoroacetic acid, to provide the title compound. MS (ESI) m/e 1338.5 (M−H)⁻.

### 2.72.2 8-[2-({[(3-amino-3-oxopropyl){2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)-3-methylbutanoyl]amino}propanoyl]amino}phenyl]-2,6-anhydro-7,8-dideoxy-L-glycero-L-gulo-octonic acid

To a solution of 2-((3S,5S)-3-(2,5-dioxo-2,5-dihydro-1H-pyrrol-1-yl)-2-oxo-5-((2-sulfoethoxy)methyl)pyrrolidin-1-yl)acetic acid (17 mg) in N,N-dimethylformamide (320 μL), was added O-(7-azabenzotriazol-1-yl)-N,N,N′,N′-tetramethyluronium hexafluorophosphate (19 mg) and N-ethyl-N-isopropylpropan-2-amine (17 μL). The reaction mixture was stirred for 5 minutes and was added to a solution of Example 2.72.1 (50 mg) and N-ethyl-N-isopropylpropan-2-amine (36 μL) in N,N-dimethylformamide (320 μL). The reaction mixture was stirred for 2 hours. The reaction mixture was diluted with N,N-dimethylformamide/water (1/1, 1 mL) and purified by reverse-phase HPLC on a Gilson system (C18 column), eluting with 20-80% acetonitrile in water containing 0.1% trifluoroacetic acid, to provide the title compound. ¹H NMR (501 MHz, dimethyl sulfoxide-d₆) δ ppm 9.82 (s, 1H), 8.15 (d, 1H), 8.00 (dd, 2H), 7.75 (d, 1H), 7.58 (d, 1H), 7.44 (ddd, 5H), 7.32 (td, 2H), 7.25 (s, 1H), 7.18 (d, 1H), 7.03 (s, 2H), 6.92 (d, 1H), 6.76 (s, 1H), 4.97 (s, 2H), 4.92 (s, 2H), 4.61 (t, 1H), 4.33 (p, 1H), 4.21-4.08 (m, 2H), 3.98 (d, 1H), 3.84 (t, 2H), 3.40-3.27 (m, 3H), 3.21 (s, 1H), 3.14-3.03 (m, 2H), 2.98 (t, 2H), 2.90 (t, 1H), 2.81-2.50 (m, 4H), 2.38-2.20 (m, 3H), 2.05 (s, 3H), 2.01-1.90 (m, 2H), 1.88-1.74 (m, 1H), 1.60-1.43 (m, 1H), 1.36-0.95 (m, 14H), 0.95-0.62 (m, 13H). MS (ESI) m/e 1697.5 (M−H)⁻.

### Example 3. Synthesis of Exemplary Bcl-xL Inhibitory ADCs

Exemplary ADCs were synthesized using one of four exemplary methods, described below. Table 1 correlates which method was used to synthesize each exemplary ADC.

Method A.

A solution of TCEP (10 mM, 0.017 mL) was added to a solution of antibody (10 mg/mL, 1 mL) preheated to 37° C. The reaction mixture was kept at 37° C. for 1 hour. The solution of reduced antibody was added to a solution of linker-warhead payload (3.3 mM, 0.160 mL in DMSO) and gently mixed for 30 minutes. The reaction solution was loaded onto a desalting column (PD10, washed with DPBS 3× before use), followed by DPBS (1.6 mL) and eluted with additional DPBS (3 mL). The purified ADC solution was filtered through a 0.2 micron, low protein-binding 13 mm syringe-filter and stored at 4° C.

Method B.

A solution of TCEP (10 mM, 0.017 mL) was added to the solution of antibody (10 mg/mL, 1 mL) preheated to 37° C. The reaction mixture was kept at 37° C. for 1 hour. The solution of reduced antibody was adjusted to pH=8 by adding boric buffer (0.05 mL, 0.5 M, pH8), added to a solution of linker-warhead payload (3.3 mM, 0.160 mL in DMSO) and gently mixed for 4 hours. The reaction solution was loaded onto a desalting column (PD 10, washed with DPBS 3× before use), followed by DPBS (1.6 mL) and eluted with additional DPBS (3 mL). The purified ADC solution was filtered through a 0.2 micron, low protein-binding 13 mm syringe-filter and stored at 4° C.

Method C.

Conjugations were performed using a PerkinElmer Janus (part AJL8M01) robotic liquid handling system equipped with an 1235/96 tip ModuLar Dispense Technology (MDT), disposable head (part 70243540) containing a gripper arm (part 7400358), and an 8-tip Varispan pipetting arm (part 7002357) on an expanded deck. The PerkinElmer Janus system was controlled using the WinPREP version 4.8.3.315 Software.

A Pall Filter plate 5052 was prewet with 100 μL 1×DPBS using the MDT. Vacuum was applied to the filter plate for 10 seconds and was followed by a 5 second vent to remove DPBS from filter plate. A 50% slurry of Protein A resin (GE MabSelect Sure) in DPBS was poured into an 8 well reservoir equipped with a magnetic ball, and the resin was mixed by passing a traveling magnet underneath the reservoir plate. The 8 tip Varispan arm, equipped with 1 mL conductive tips, was used to aspirate the resin (250 μL) and transfer to a 96-well filter plate. A vacuum was applied for 2 cycles to remove most of the buffer. Using the MDT, 150 μL of 1×PBS was aspirated and dispensed to the 96-well filter plate holding the resin. A vacuum was applied, removing the buffer from the resin. The rinse/vacuum cycle was repeated 3 times. A 2 mL, 96-well collection plate was mounted on the Janus deck, and the MDT transferred 450 μL of 5×DPBS to the collection plate for later use. Reduced antibody (2 mg) as a solution in (200 μL) DPBS was prepared as described above for Conditions A and preloaded into a 96 well plate. The solutions of reduced antibody were transferred to the filter plate wells containing the resin, and the mixture was mixed with the MDT by repeated aspiration/dispensation of a 100 μL volume within the well for 45 seconds per cycle. The aspiration/dispensation cycle was repeated for a total of 5 times over the course of 5 minutes. A vacuum was applied to the filter plate for 2 cycles, thereby removing excess antibody. The MDT tips were rinsed with water for 5 cycles (200 μL, 1 mL total volume). The MDT aspirated and dispensed 150 μL of DPBS to the filter plate wells containing resin-bound antibody, and a vacuum was applied for two cycles. The wash and vacuum sequence was repeated two more times. After the last vacuum cycle, 100 μL of 1×DPBS was dispensed to the wells containing the resin-bound antibody. The MDT then collected 30 μL each of 3.3 mM dimethyl sulfoxide solutions of synthons plated in a 96-well format and dispensed it to the filter plate containing resin-bound antibody in DPBS. The wells containing the conjugation mixture were mixed with the MDT by repeated aspiration/dispensation of a 100 μL volume within the well for 45 seconds per cycle. The aspiration/dispensation sequence was repeated for a total of 5 times over the course of 5 minutes. A vacuum was applied for 2 cycles to remove excess synthon to waste. The MDT tips were rinsed with water for 5 cycles (200 μL. 1 mL total volume). The MDT aspirated and dispensed DPBS (150 μL) to the conjugation mixture, and a vacuum was applied for two cycles. The wash and vacuum sequence was repeated two more times. The MDT gripper then moved the filter plate and collar to a holding station. The MDT placed the 2 mL collection plate containing 450 μL of 10×DPBS inside the vacuum manifold. The MDT reassembled the vacuum manifold by placement of the filter plate and collar. The MDT tips were rinsed with water for 5 cycles (200 μL, 1 mL total volume). The MDT aspirated and dispensed 100 μL of IgG Elution Buffer 3.75 (Pierce) to the conjugation mixture. After one minute, a vacuum was applied for 2 cycles, and the eluent was captured in the receiving plate containing 450 μL of 5×DPBS. The aspiration/dispensation sequence was repeated 3 additional times to deliver ADC samples with concentrations in the range of 1.5-2.5 mg/mL at pH 7.4 in DPBS.

Method D.

Conjugations were performed using a PerkinElmer Janus (part AJL8M01) robotic liquid handling system equipped with an 1235/96 tip ModuLar Dispense Technology (MDT), disposable head (part 70243540) containing a gripper arm (part 7400358), and an 8-tip Varispan pipetting arm (part 7002357) on an expanded deck. The PerkinElmer Janus system was controlled using the WinPREP version 4.8.3.315 Software.

A Pall Filter plate 5052 was prewet with 100 μL 1×DPBS using the MDT. Vacuum was applied to the filter plate for 10 seconds and was followed by a 5 second vent to remove DPBS from filter plate. A 50% slurry of Protein A resin (GE MabSelect Sure) in DPBS was poured into an 8-well reservoir equipped with a magnetic ball, and the resin was mixed by passing a traveling magnet underneath the reservoir plate. The 8 tip Varispan arm, equipped with 1 mL conductive tips, was used to aspirate the resin (250 μL) and transfer to a 96-well filter plate. A vacuum was applied to the filter plate for 2 cycles to remove most of the buffer. The MDT aspirated and dispensed 150 μL of DPBS to the filter plate wells containing the resin. The wash and vacuum sequence was repeated two more times. A 2 mL, 96-well collection plate was mounted on the Janus deck, and the MDT transferred 450 μL of 5×DPBS to the collection plate for later use. Reduced antibody (2 mg) as a solution in (200 μL) DPBS was prepared as described above for Conditions A and dispensed into the 96-well plate. The MDT then collected 30 μL each of 3.3 mM dimethyl sulfoxide solutions of synthons plated in a 96-well format and dispensed it to the plate loaded with reduced antibody in DPBS. The mixture was mixed with the MDT by twice repeated aspiration/dispensation of a 100 μL volume within the well. After five minutes, the conjugation reaction mixture (230 μL) was transferred to the 96-well filter plate containing the resin. The wells containing the conjugation mixture and resin were mixed with the MDT by repeated aspiration/dispensation of a 100 μL volume within the well for 45 seconds per cycle. The aspiration/dispensation sequence was repeated for a total of 5 times over the course of 5 minutes. A vacuum was applied for 2 cycles to remove excess synthon and protein to waste. The MDT tips were rinsed with water for 5 cycles (200 μL, 1 mL total volume). The MDT aspirated and dispensed DPBS (150 μL) to the conjugation mixture, and a vacuum was applied for two cycles. The wash and vacuum sequence was repeated two more times. The MDT gripper then moved the filter plate and collar to a holding station. The MDT placed the 2 mL collection plate containing 450 L of 10×DPBS inside the vacuum manifold. The MDT reassembled the vacuum manifold by placement of the filter plate and collar. The MDT tips were rinsed with water for 5 cycles (200 μL, 1 mL total volume). The MDT aspirated and dispensed 100 μL of IgG Elution Buffer 3.75 (P) to the conjugation mixture. After one minute, a vacuum was applied for 2 cycles, and the eluent was captured in the receiving plate containing 450 μL of 5×DPBS. The aspiration/dispensation sequence was repeated 3 additional times to deliver ADC samples with concentrations in the range of 1.5-2.5 mg/mL at pH 7.4 in DPBS.

Method E.

A solution of TCEP (10 mM, 0.017 mL) was added to the solution of antibody (10 mg/mL, 1 mL) at room temperature. The reaction mixture was heated to 37° C. for 75 minutes. The solution of reduced antibody cooled to room temperature and was added to a solution of synthon (10 mM, 0.040 mL in DMSO) followed by addition of boric buffer (0.1 mL, 1M, pH 8). The reaction solution was let to stand for 3 days at room temperature, loaded onto a desalting column (PD 10, washed with DPBS 3×5 mL before use), followed by DPBS (1.6 mL) and eluted with additional DPBS (3 mL). The purified ADC solution was filtered through a 0.2 micron, low protein-binding 13 mm syringe-filter and stored at 4 C.

Method F.

Conjugations were performed using a Tecan Freedom Evo robotic liquid handling system. The solution of antibody (10 mg/mL) was preheated to 37° C. and aliquoted to a heated 96 deep-well plate in amounts of 3 mg per well (0.3 mL) and kept at 37° C. A solution of TCEP (1 mM, 0.051 mL/well) was added to antibodies, and the reaction mixture was kept at 37° C. for 75 minutes. The solution of reduced antibody was transferred to an unheated 96 deep-well plate. Corresponding solutions of synthons (5 mM, 0.024 mL in DMSO) were added to the wells with reduced antibodies and treated for 15 minutes. The reaction solutions were loaded onto a platform (8×12) of desalting columns (NAPS, washed with DPBS 4× before use), followed by DPBS (0.3 mL) and eluted with additional DPBS (0.8 mL). The purified ADC solutions were further aliquoted for analytics and stored at 4° C.

Method G.

Conjugations were performed using a Tecan Freedom Evo robotic liquid handling system. The solution of antibody (10 mg/mL) was preheated to 37° C. and aliquoted onto a heated 96 deep-well plate in amounts of 3 mg per well (0.3 mL) and kept at 37° C. A solution of TCEP (1 mM. 0.051 mL/well) was added to antibodies, and the reaction mixture was kept at 37° C. for 75 minutes. The solutions of reduced antibody were transferred to an unheated 96 deep-well plate. Corresponding solutions of synthons (5 mM, 0.024 mL/well in DMSO) were added to the wells with reduced antibodies followed by addition of boric buffer (pH=8. 0.03 mL/well) and treated for 3 days. The reaction solutions were loaded onto a platform (8×12) of desalting columns (NAPS, washed with DPBS 4× before use), followed by DPBS (0.3 mL) and eluted with additional DPBS (0.8 mL). The purified ADC solutions were further aliquoted for analytics and stored at 4° C.

Method H.

A solution of TCEP (10 mM, 0.17 mL) was added to the solution of antibody (10 mg/mL, 10 mL) at room temperature. The reaction mixture was heated to 37° C. for 75 minutes. The solution of synthon (10 mM, 0.40 mL in DMSO) was added to a solution of reduced antibody cooled to room temperature. The reaction solution was let to stand for 30 minutes at room temperature. The solution of ADC was treated with saturated ammonium sulfate solution (˜2-2.5 mL) until a slightly cloudy solution formed. This solution was loaded onto butyl sepharose column (5 mL of butyl sepharose) equilibrated with 30% phase B in phase A (phase A: 1.5 M ammonium sulfate, 25 mM phosphate; phase B: 25 mM phosphate, 25% isopropanol v/v). Individual fractions with DAR2 (also referred to as “E2”) and DAR4 (also referred to as “E4”) eluted upon applying gradient A/B up to 75% phase B. Each ADC solution was concentrated and buffer switched using centrifuge concentrators or TFF for larger scales. The purified ADC solutions were filtered through a 0.2 micron, low protein-binding 13 mm syringe-filter and stored at 4 C.

Table 1, below, indicates which exemplary ADCs were synthesized via which exemplary method. The NCAM-1 antibody referred to as N901 is described in Roguska et al., 1994, Proc Natl Acad Sci USA 91:969-973. The EGFR antibody referred to as AB033 is described in WO 2009/134776 (see page 120).

TABLE 1
Synthetic Methods Used to Synthesize Exemplary ADCs

| Appln Ex. No. | ADC | Method |
|---|---|---|
| 3.1 | AB033-BS | A |
| 3.2 | AB033-DK | A |
| 3.3 | AB033-DQ | A |
| 3.4 | Ab033-DJ | A |
| 3.5 | AB033-DO | A |
| 3.6 | AB033-DP | A |
| 3.7 | AB033-HO | A |
| 3.8 | AB033-KA | A |
| 3.9 | AB033-KB | A |
| 3.10 | AB033-KT | A |
| 3.11 | AB033-KU | D |
| 3.12 | AB033-KV | D |
| 3.13 | AB033-KW | A |
| 3.14 | AB033-KZ | A |
| 3.15 | AB033-LW | D |
| 3.16 | AB033-LY | D |
| 3.17 | AB033-LZ | D |
| 3.18 | AB033-MB | D |
| 3.19 | AB033-MC | D |
| 3.20 | AB033-ME | D |
| 3.21 | AB033-MF | D |
| 3.22 | AB033-MH | D |
| 3.23 | AB033-MI | D |
| 3.24 | AB033-NJ | D |
| 3.25 | AB033-NK | D |
| 3.26 | AB033-NL | D |
| 3.27 | AB033-NM | D |
| 3.28 | AB033-NR | A |
| 3.33 | N901-KA | A |
| 3.34 | N901-KB | A |
| 3.35 | AB033-EB | A |
| 3.36 | AB033-DC | A |
| 3.37 | MSL109-KA | D |
| 3.38 | MSL109-KB | D |
| 3.39 | AB033-OG | D |
| 3.40 | AB033-OH | A |
| 3.41 | AB033-ON | A |
| 3.42 | AB033-OT | A |
| 3.43 | AB033-OP | A |
| 3.44 | AB033-OU | A |
| 3.45 | AB033-OO | A |
| 3.46 | AB033-OQ | A |
| 3.47 | AB033-OR | A |
| 3.48 | AB033-OS | A |
| 3.49 | AB033-PA | D |
| 3.50 | AB033-QL | D |
| 3.51 | AB033-QM | D |
| 3.52 | AB033-QN | D |
| 3.53 | AB033-QT | D |
| 3.54 | AB033-RF | D |
| 3.55 | AB033-RG | D |
| 3.56 | AB033-SF | A |
| 3.57 | AB033-SR | A |
| 3.58 | AB033-YZ | G |
| 3.59 | AB033-QR | D |
| 3.60 | AB033-SE | A |
| 3.61 | AB033-UH | E |
| 3.62 | AB033-UI | E |
| 3.63 | AB033-US | E |
| 3.64 | AB033-UY | E |
| 3.65 | AB033-UX | E |
| 3.66 | AB033-WZ | G |
| 3.67 | AB033-XO | E |
| 3.68 | AB033-XW | E |
| 3.69 | AB033-YG | G |
| 3.70 | AB033-ZT | G |
| 3.71 | AB033-AAN | G |
| 3.72 | AB033-AAO | G |
| 3.73 | AB033-AAP | G |
| 3.74 | AB033-ZZ | G |

### Example 4. Exemplary Bcl-xL Inhibitors Bind Bcl-xL

The ability of the exemplary Bcl-xL inhibitors of Examples 1.1 through 1.18 (compounds W3.01-W3.18 respectively) to bind Bcl-xL was demonstrated using the Time Resolved-Fluorescence Resonance Energy Transfer (TR-FRET) Assay. Tb-anti-GST antibody was purchased from Invitrogen (Catalog No. PV4216).

### 4.1. Probe Synthesis

### 4.1.1. Reagents

All reagents were used as obtained from the vendor unless otherwise specified. Peptide synthesis reagents including diisopropylethylamine (DIEA), dichloromethane (DCM), N-methylpyrrolidone (NMP), 2-(1H-benzotriazole-1-yl)-1,1,3,3-tetramethyluronium hexafluorophosphate (HBTU), N-hydroxybenzotriazole (HOBt) and piperidine were obtained from Applied Biosystems, Inc. (ABI), Foster City, Calif. or American Bioanalytical, Natick, Mass.

Preloaded 9-Fluorenylmethyloxycarbonyl (Fmoc) amino acid cartridges (Fmoc-Ala-OH. Fmoc-Cys(Trt)-OH, Fmoc-Asp(tBu)-OH, Fmoc-Glu(tBu)-OH, Fmoc-Phe-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Asn(Trt)-OH, Fmoc-Pro-OH, Fmor-Gln(Trt)-OH, Fmoc-Arg(Pbf)-OH, Fmoc-Ser(tBu)-OH, Fmoc-Thr(tBu)-OH, Fmoc-Val-OH, Fmoc-Trp(Boc)-OH, Fmoc-Tyr(tBu)-OH) were obtained from ABI or Anaspec, San Jose, Calif.

The peptide synthesis resin (Fmoc-Rink amide MBHA resin) and Fmoc-Lys(Mtt)-OH were obtained from Novabiochem, San Diego, Calif.

Single-isomer 6-carboxyfluorescein succinimidyl ester (6-FAM-NHS) was obtained from Anaspec.

Trifluoroacetic acid (TFA) was obtained from Oakwood Products, West Columbia, S.C.

Thioanisole, phenol, triisopropylsilane (TIS), 3,6-dioxa-1,8-octanedithiol (DODT) and isopropanol were obtained from Aldrich Chemical Co., Milwaukee, Wis.

Matrix-assisted laser desorption ionization mass-spectra (MALDI-MS) were recorded on an Applied Biosystems Voyager DE-PRO MS).

Electrospray mass-spectra (ESI-MS) were recorded on Finnigan SSQ7000 (Finnigan Corp., San Jose. Calif.) in both positive and negative ion mode.

### 4.1.2. General Procedure for Solid-Phase Peptide Synthesis (SPPS)

Peptides were synthesized with, at most, 250 μmol preloaded Wang resin/vessel on an ABI 433A peptide synthesizer using 250 μmol scale Fastmoc™ coupling cycles. Preloaded cartridges containing 1 mmol standard Fmoc-amino acids, except for the position of attachment of the fluorophore, where 1 mmol Fmoc-Lys(Mtt)-OH was placed in the cartridge, were used with conductivity feedback monitoring. N-terminal acetylation was accomplished by using 1 mmol acetic acid in a cartridge under standard coupling conditions.

### 4.1.3. Removal of 4-Methyltrityl (Mtt) from Lysine

The resin from the synthesizer was washed thrice with dichloromethane and kept wet. 150 mL of 95:4:1 dichloromethane:triisopropylsilane:trifluoroacetic acid was flowed through the resin bed over 30 minutes. The mixture turned deep yellow then faded to pale yellow. 100 mL of DMF was flowed through the bed over 15 minutes. The resin was then washed thrice with DMF and filtered. Ninhydrin tests showed a strong signal for primary amine.

### 4.1.4. Resin Labeling with 6-Carboxyfluorescein-NHS (6-FAM-NHS)

The resin was treated with 2 equivalents 6-FAM-NHS in 1% DIEA/DMF and stirred or shaken at ambient temperature overnight. When complete, the resin was drained, washed thrice with DMF, thrice with (1× dichloromethane and 1× methanol) and dried to provide an orange resin that was negative by ninhydrin test.

### 4.1.5. General Procedure for Cleavage and Deprotection of Resin-Bound Peptide

Peptides were cleaved from the resin by shaking for 3 hours at ambient temperature in a cleavage cocktail consisting of 80% TFA, 5% water, 5% thioanisole, 5% phenol, 2.5% TIS, and 2.5% EDT (1 mL/0.1 g resin). The resin was removed by filtration and rinsing twice with TFA. The TFA was evaporated from the filtrates, and product was precipitated with ether (10 mL/0.1 g resin), recovered by centrifugation, washed twice with ether (10 mL/0.1 g resin) and dried to give the crude peptide.

### 4.1.6. General Procedure for Purification of Peptides

The crude peptides were purified on a Gilson preparative HPLC system running Unipoint® analysis software (Gilson, Inc., Middleton, Wis.) on a radial compression column containing two 25×100 mm segments packed with Delta-Pak™ C18 15 μm particles with 100 Å pore size and eluted with one of the gradient methods listed below. One to two milliliters of crude peptide solution (10 mg/mL in 90% DMSO/water) was purified per injection. The peaks containing the product(s) from each run were pooled and lyophilized. All preparative runs were run at 20 mL/min with eluents as buffer A: 0.10% TFA-water and buffer B: acetonitrile.

### 4.1.7. General Procedure for Analytical HPLC

Analytical HPLC was performed on a Hewlett-Packard 1200 series system with a diode-array detector and a Hewlett-Packard 1046A fluorescence detector running HPLC 3D ChemStation software version A.03.04 (Hewlett-Packard. Palo Alto, Calif.) on a 4.6×250 mm YMC column packed with ODS-AQ 5 μm particles with a 120 Å pore size and eluted with one of the gradient methods listed below after preequilibrating at the starting conditions for 7 minutes. Eluents were buffer A: 0.1% TFA-water and buffer B: acetonitrile. The flow rate for all gradients was 1 mL/min.

### 4.1.8. Synthesis of Probe F-Bak

Peptide probe F-bak, which binds Bcl-xL, was synthesized as described below. Probe F-Bak is acetylated at the N-terminus, amidated at the C-terminus and has the amino acid sequence GQVGRQLAIIGDKINR (SEQ ID NO: 1). It is fluoresceinated at the lysine residue (K) with 6-FAM. Probe F-Bak can be abbreviated as follows: acetyl-GQVGRQLAIIGDK(6-FAM)INR-NH₂.

To make probe F-Bak, Fmoc-Rink amide MBHA resin was extended using the general peptide synthesis procedure to provide the protected resin-bound peptide (1.020 g). The Mtt group was removed, labeled with 6-FAM-NHS and cleaved and deprotected as described hereinabove to provide the crude product as an orange solid (0.37 g). This product was purified by RP-HPLC. Fractions across the main peak were tested by analytical RP-HPLC, and the pure fractions were isolated and lyophilized, with the major peak providing the title compound (0.0802 g) as a yellow solid; MALDI-MS m/z=2137.1 [(M+H)⁺].

### 4.1.9. Alternative Synthesis of Peptide Probe F-Bak

In an alternative method, the protected peptide was assembled on 0.25 mmol Fmoc-Rink amide MBHA resin (Novabiochem) on an Applied Biosystems 433A automated peptide synthesizer running Fastmoc™ coupling cycles using pre-loaded 1 mmol amino acid cartridges, except for the fluorescein(6-FAM)-labeled lysine, where 1 mmol Fmoc-Lys(4-methyltrityl) was weighed into the cartridge. The N-terminal acetyl group was incorporated by putting 1 mmol acetic acid in a cartridge and coupling as described hereinabove. Selective removal of the 4-methyltrityl group was accomplished with a solution of 95:4:1 DCM:TIS:TFA (v/v/v) flowed through the resin over 15 minutes, followed by quenching with a flow of dimethylformamide. Single-isomer 6-carboxyfluorescein-NHS was reacted with the lysine side-chain in 1% DIEA in DMF and confirmed complete by ninhydrin testing. The peptide was cleaved from the resin and side-chains deprotected by treating with 80:5:5:5:2.5:2.5 TFA/water/phenol/thioanisole/triisopropylsilane: 3,6-dioxa-1,8-octanedithiol (v/v/v/v/v/v), and the crude peptide was recovered by precipitation with diethyl ether. The crude peptide was purified by reverse-phase high-performance liquid chromatography, and its purity and identity were confirmed by analytical reverse-phase high-performance liquid chromatography and matrix-assisted laser-desorption mass-spectrometry (m/z=2137.1 ((M+H)⁺).

### 4.2. Time Resolved-Fluorescence Resonance Energy Transfer (TR-FRET) Assay

The ability of exemplary Bcl-xL inhibitors W3.01-W3.18 to compete with probe F-Bak for binding Bcl-xL was demonstrated using a Time Resolved Fluorescence Resonance Energy Transfer (TR-FRET) binding assay.

### 4.2.1. Method

For the assay, test compounds were serially diluted in DMSO starting at 50 μM (2× starting concentration; 10% DMSO) and 10 μL transferred into a 384-well plate. 10 μL of a protein/probe/antibody mix was then added to each well at final concentrations listed below:

| | | |
|---|---|---|
| Protein: | GST-Bcl-xL | 1 nM |
| Antibody | Tb-anti-GST | 1 nM |
| Probe: | F-Bak | 100 nM |

The samples were then mixed on a shaker for 1 minute and incubated for an additional 2 hours at room temperature. For each assay plate, a probe/antibody and protein/antibody/probe mixture were included as a negative and a positive control, respectively. Fluorescence was measured on the Envision (Perkin Elmer) using a 340/35 nm excitation filter and 520/525 (F-Bak) and 495/510 nm (Tb-labeled anti-his antibody) emission filters. Dissociation constants (Kᵢ) were determined using Wang's equation (Wang, 1995, FEBS Lett. 360:111-114). The TR-FRET assay can be performed in the presence of varying concentrations of human serum (HS) or fetal bovine serum (FBS). Compounds were tested both without HS and in the presence of 1% HS.

### 4.2.2. Results

The results of binding assays (Kᵢ in nanomolar) are provided in Table 2, below:

TABLE 2
TR-FRET Bcl-xL Binding Data

| | | Bcl-xL | Bcl-xL |
|---|---|---|---|
| | | Binding | Binding |
| Appln | | Kᵢ | Kᵢ |
| Ex. No. | Cmpd | (nM) | (nM, 1% HS) |
| 1.1 | W3.01 | <0.001 | 0.009 |
| 1.2 | W3.02 | <0.001 | 0.047 |
| 1.3 | W3.03 | <0.001 | 0.019 |
| 1.4 | W3.04 | <0.001 | 0.049 |
| 1.5 | W3.05 | 0.02 | 0.23 |
| 1.6 | W3.06 | <0.001 | 0.22 |
| 1.7 | W3.07 | <0.001 | 0.29 |
| 1.8 | W3.08 | <0.001 | 0.013 |
| 1.9 | W3.09 | <0.001 | 0.14 |
| 1.10 | W3.10 | <0.001 | 0.0259 |
| 1.11 | W3.11 | <0.001 | 0.94 |
| 1.12 | W3.12 | 0.0042 | 0.051 |
| 1.13 | W3.13 | 0.013 | 6.8 |
| 1.14 | W3.14 | <0.001 | 0.014 |
| 1.15 | W3.15 | <0.001 | 0.1 |
| 1.16 | W3.16 | <0.001 | 0.14 |
| 1.17 | W3.17 | 0.49 | 2.3 |
| 1.18 | W3.18 | 0.038 | 0.19 |
| 1.19 | W3.19 | 21 | 309 |
| 1.20 | W3.20 | <0.01 | 0.014 |
| 1.21 | W3.21 | 0.014 | 0.14 |
| 1.22 | W3.22 | <0.01 | 0.108 |
| 1.23 | W3.23 | 0.021 | 1.1 |
| 1.24 | W3.24 | 0.794 | 8.14 |
| 1.25 | W3.25 | 0.138 | 0.9 |
| 1.26 | W3.26 | <0.02 | 0.083 |
| 1.27 | W3.27 | NV | 0.12 |
| 1.28 | W3.28 | <.01 | 0.17 |
| 1.29 | W3.29 | <0.01 | 0.09 |
| 1.30 | W3.30 | 0.011 | 0.891 |
| 1.31 | W3.31 | 0.012 | 0.684 |
| 1.32 | W3.32 | <0.01 | 0.365 |
| 1.33 | W3.33 | 0.044 | 0.319 |
| 1.34 | W3.34 | 0.041 | 0.27 |
| 1.35 | W3.35 | 0.022 | 0.16 |
| 1.36 | W3.36 | NT | NT |
| 1.37 | W3.37 | 0.03 | 0.58 |
| 1.38 | W3.38 | NT | NT |
| 1.39 | W3.39 | 0.015 | 0.44 |
| 1.40 | W3.40 | 0.024 | 1.1 |
| 1.41 | W3.41 | NT | NT |
| 1.42 | W3.42 | 0.15 | 4.36 |
| 1.43 | W3.43 | <0.01 | 0.07 |

NT = not tested,
NV = not valid

In one exemplary set of conditions, Molt-4 (ATCC. Manassas, Va.) human acute lymphoblastic leukemia cells were plated 12,500 cells per well in 384-well tissue culture plates (Corning, Corning, N.Y.) in a total volume of 25 μL tissue culture medium supplemented with 10% human serum (Sigma-Aldrich, St. Louis, Mo.) and treated with a 3-fold serial dilution of the compounds of interest from 10 μM to 0.0005 μM. Each concentration was tested in duplicate at least 3 separate times. The number of viable cells following 48 hours of compound treatment was determined using the CellTiter-Glo® Luminescent Cell Viability Assay according to the manufacturer's recommendations (Promega Corp., Madison, Wis.). Compounds were tested in the presence of 10% HS.

### 5.2. Results

The results of a Molt-4 cell viability assay (EC₅₀ in nanomolar) carried out in the presence of 10% HS for exemplary Bcl-xL inhibitors of Examples 1.1-1.43 (compounds W3.01-W3.43, respectively) are provided in Table 3, below (Bcl-xL binding data of Table 2 are repeated in Table 3).

TABLE 3
Bcl-xL Inhibitor In Vitro Data

| | | Bcl-xL | Bcl-xL | Molt-4 |
|---|---|---|---|---|
| | | Binding | Binding | Viability |
| | | Kᵢ | Kᵢ | EC₅₀ |
| Ex. No. | Cmpd | (nM) | (nM, 1% HS) | (nM, 10% HS) |
| 1.1 | W3.01 | <0.001 | 0.009 | 0.3 |
| 1.2 | W3.02 | <0.001 | 0.047 | 0.5 |
| 1.3 | W3.03 | <0.001 | 0.019 | 1.4 |
| 1.4 | W3.04 | <0.001 | 0.049 | 58.9 |
| 1.5 | W3.05 | 0.02 | 0.23 | 79 |
| 1.6 | W3.06 | <0.001 | 0.22 | 3.8 |
| 1.7 | W3.07 | <0.001 | 0.29 | 432 |
| 1.8 | W3.08 | <0.001 | 0.013 | 40 |
| 1.9 | W3.09 | <0.001 | 0.14 | 3.8 |
| 1.10 | W3.10 | <0.001 | 0.0259 | NT |
| 1.11 | W3.11 | <0.001 | 0.94 | 34.3 |
| 1.12 | W3.12 | 0.0042 | 0.051 | 2.6 |
| 1.13 | W3.13 | 0.013 | 6.8 | 2290 |
| 1.14 | W3.14 | <0.001 | 0.014 | 1.8 |
| 1.15 | W3.15 | <0.001 | 0.1 | 2.5 |
| 1.16 | W3.16 | <0.001 | 0.14 | 3.7 |
| 1.17 | W3.17 | 0.49 | 2.3 | NT |
| 1.18 | W3.18 | 0.038 | 0.19 | 14 |
| 1.19 | W3.19 | 21 | 309 | >10,000 |
| 1.20 | W3.20 | <0.01 | 0.014 | 18.2 |
| 1.21 | W3.21 | 0.014 | 0.14 | NT |
| 1.22 | W3.22 | <0.01 | 0.108 | NT |
| 1.23 | W3.23 | 0.021 | 1.1 | NT |
| 1.24 | W3.24 | 0.794 | 8.14 | 2,210 |
| 1.25 | W3.25 | 0.138 | 0.9 | 424 |
| 1.26 | W3.26 | <0.02 | 0.083 | 4.3 |
| 1.27 | W3.27 | NV | 0.12 | 3.95 |
| 1.28 | W3.28 | <.01 | 0.17 | 8.38 |
| 1.29 | W3.29 | <0.01 | 0.09 | 185 |
| 1.30 | W3.30 | 0.011 | 0.891 | 16.3 |
| 1.31 | W3.31 | 0.012 | 0.684 | 14.4 |
| 1.32 | W3.32 | <0.01 | 0.365 | 108 |
| 1.33 | W3.33 | 0.044 | 0.319 | 422 |
| 1.34 | W3.34 | 0.041 | 0.27 | 187 |
| 1.35 | W3.35 | 0.022 | 0.16 | 658 |
| 1.36 | W3.36 | NT | NT | 6.9 |
| 1.37 | W3.37 | 0.03 | 0.58 | 10.8 |
| 1.38 | W3.38 | NT | NT | 10.7 |
| 1.39 | W3.39 | 0.015 | 0.44 | 37.7 |
| 1.40 | W3.40 | 0.024 | 1.1 | NT |
| 1.41 | W3.41 | NT | NT | NT |
| 1.42 | W3.42 | 0.15 | 4.36 | NT |
| 1.43 | W3.43 | <0.01 | 0.07 | NT |

NT = not tested,
NV = not valid

### Example 6. DAR and Aggregation of Exemplary ADCs

The DAR and percentage aggregation of exemplary ADCs synthesized as described in Example 3, above, were determined by LC-MS and size exclusion chromatography (SEC), respectively.

### 6.1. LC-MS General Methodology

LC-MS analysis was performed using an Agilent 1100 HPLC system interfaced to an Agilent LC/MSD TOF 6220 ESI mass spectrometer. The ADC was reduced with 5 mM (final concentration) Bond-Breaker® TCEP solution (Thermo Scientific, Rockford, Ill.), loaded onto a Protein Microtrap (Michrom Bioresorces, Auburn, Calif.) desalting cartridge, and eluted with a gradient of 10% B to 75% B in 0.2 minutes at ambient temperature. Mobile phase A was H20 with 0.1% formic acid (FA), mobile phase B was acetonitrile with 0.1% FA, and the flow rate was 0.2 ml/min. Electrospray-ionization time-of-flight mass spectra of the co-eluting light and heavy chains were acquired using Agilent MassHunter™ acquisition software. The extracted intensity vs. m/z spectrum was deconvoluted using the Maximum Entropy feature of MassHunter software to determine the mass of each reduced antibody fragment. DAR was calculated from the deconvoluted spectrum by summing intensities of the naked and modified peaks for the light chain and heavy chain, normalized by multiplying intensity by the number of drugs attached. The summed, normalized intensities were divided by the sum of the intensities, and the summing results for two light chains and two heavy chains produced a final average DAR value for the full ADC.

### 6.2. Size Exclusion Chromatography General Methodology

Size exclusion chromatography was performed using a Shodex KW802.5 column in 0.2M potassium phosphate pH 6.2 with 0.25 mM potassium chloride and 15% IPA at a flow rate of 0.75 ml/min. The peak area absorbance at 280 nm was determined for each of the high molecular weight and monomeric eluents by integration of the area under the curve. The % aggregate fraction of the conjugate sample was determined by dividing the peak area absorbance at 280 nM for the high molecular weight eluent by the sum of the peak area absorbances at 280 nM of the high molecular weight and monomeric eluents multiplied by 100%.

### 6.3. Results

The average DAR values determined by the above LC-MS method and the % aggregate fraction for the exemplary ADCs are reported in Table 4:

TABLE 4
ADC Analytical Characterization

| Appln | | % Agg | DAR |
|---|---|---|---|
| Ex. No. | ADC Code | (by SEC) | (by MS) |
| 3.1 | AB033-BS | 13.8 | 2.2 |
| 3.2 | AB033-DK | 46 | 4.1 |
| 3.3 | AB033-DQ | 56 | 4.2 |
| 3.4 | Ab033-DJ | 3.3 | 4 |
| 3.5 | AB033-DO | 4.3 | 4.2 |
| 3.6 | AB033-DP | 2.9 | 4.1 |
| 3.7 | AB033-HO | 12 | 2.73 |
| 3.8 | AB033-KA | 10 | 3.9 |
| 3.9 | AB033-KB | 16.7 | 3.7 |
| 3.10 | AB033-KT | 6.8 | 3.6 |
| 3.11 | AB033-KU | 6.7 | 3.4 |
| 3.12 | AB033-KV | 3.5 | |
| 3.13 | AB033-KW | 7.3 | 3.8 |
| 3.14 | AB033-KZ | 9.7 | 3.96 |
| 3.15 | AB033-LW | 25.8 | 4.2 |
| 3.16 | AB033-LY | 12 | 3.1 |
| 3.17 | AB033-LZ | 9.1 | 3.7 |
| 3.18 | AB033-MB | 25 | 3.3 |
| 3.19 | AB033-MC | 21.6 | 4 |
| 3.20 | AB033-ME | 5.2 | 2.1 |
| 3.21 | AB033-MF | 4.8 | 3 |
| 3.22 | AB033-MH | 9.4 | |
| 3.23 | AB033-MI | 9.1 | 3.1 |
| 3.24 | AB033-NJ | 4.4 | 3 |
| 3.25 | AB033-NK | 3.7 | 3.1 |
| 3.26 | AB033-NL | 4.1 | 2.9 |
| 3.27 | AB033-NM | 4.5 | 3 |
| 3.28 | AB033-NR | 9.2 | 0.01 |
| 3.33 | N901-KA | 8.8 | 2.9 |
| 3.31 | N901-KB | 15.3 | 3 |
| 3.35 | AB033-EB | 31 | 3.6 |
| 3.36 | AB033-DC | 6.4 | 3.5 |
| 3.37 | MSL109-KA | 19.7 | 3.9 |
| 3.38 | MSL109-KB | 34.7 | 4 |
| 3.39 | AB033-OG | 3.6 | 2.6 |
| 3.40 | AB033-OH | 1.6 | 3.3 |
| 3.41 | AB033-ON | 3.0 | 2.9 |
| 3.42 | AB033-OT | 2.6 | 3.1 |
| 3.43 | AB033-OP | 1.6 | 3.4 |
| 3.44 | AB033-OU | 3.2 | 3.2 |
| 3.45 | AB033-OO | 3.9 | 2.8 |
| 3.46 | AB033-OQ | 2.9 | 3.3 |
| 3.47 | AB033-OR | 2.6 | 3.0 |
| 3.48 | AB033-OS | 2.9 | 2.52 |
| 3.49 | AB033-PA | 2.5 | 0.87 |
| 3.50 | AB033-QL | 1.4 | 1.3 |
| 3.51 | AB033-QM | 1.5 | 0.67 |
| 3.52 | AB033-QN | 1.0 | 1.72 |
| 3.53 | AB033-QT | 10.11 | 2.33 |
| 3.54 | AB033-RF | 6.66 | 0.87 |
| 3.55 | AB033-RG | 4.8 | 1.88 |
| 3.56 | AB033-SF | 30.0 | 2.3 |
| 3.57 | AB033-SR | 33.2 | 2.7 |
| 3.58 | AB033-YZ | 5.7 | 3.5 |
| 3.59 | AB033-QR | 2.0 | 3.33 |
| 3.60 | AB033-SE | 0.6 | 3.1 |
| 3.61 | AB033-UH | 6.1 | 3.9 |
| 3.62 | AB033-UI | 2.7 | 4.0 |
| 3.63 | AB033-US | 8.4 | 3.4 |
| 3.64 | AB033-UY | 2.7 | 4.2 |
| 3.65 | AB033-UX | 3.1 | 4.6 |
| 3.66 | AB033-WZ | 12.5 | 3.4 |
| 3.67 | AB033-XO | 7.4 | 4.1 |
| 3.68 | AB033-XW | 5.0 | 4.4 |
| 3.69 | AB033-YG | 3.7 | 4.6 |
| 3.70 | AB033-ZT | 5 | 4 |
| 3.71 | AB033-AAN | 3.5 | 5.3 |
| 3.72 | AB033-AAO | 1.6 | 5.3 |
| 3.73 | AB033-AAP | 4.5 | 4.6 |
| 3.74 | AB033-ZZ | 2.3 | 4 |

### Example 7. EGFR-Targeted ADCs Inhibit the Growth of Cancer Cells in Vitro

### 7.1. Certain Exemplary ADCs Comprising Antibody AB033 was Evaluated. Antibody AB033 Targets Human EGFR. The Variable Heavy and Light Chain Sequences of Antibody AB033 are Described in WO 2009/134776 (See Page 120). The Ability of Antibody AB033 to Inhibit the Growth of Cancer Cells was Demonstrated with Mcl-1^{-/-} Mouse Embryonic Fibroblast (MEF) Cells. Mcl-1^{-/-} MEFs are Dependent Upon Bcl-xL for Survival (Lessene et al., 2013, Nature Chemical Biology 9:390-397). To Evaluate the Efficacy of Exemplary AB033-Targeted Bcl-xL-ADCs, Human ECFR was Over-Expressed in Mcl-1^{-/-} MEFs. Mcl-1^{-/-} MEFs were Obtained from David C. S. Huang of the Walter and Eliza Hall Institute of Medical Research. Method

Retroviral supernatants were produced through transfection of the GP2-293 packaging cell line (Clontech) with the retroviral construct pLVC-IRES-Hygro (Clontech) containing huEGFR sequence or the empty vector utilizing FuGENE 6 transfection reagent (Roche Molecular Biochemicals, Mannheim, Germany). After 48 hours of culture, virus-containing supernatant was harvested and applied to mcl-1^{-/-} MEFs in 75 cm² culture flasks (0.5×10⁶ per flask) for a further 48 hours in the presence of polybrene (8 μg/ml; Sigma). Mcl-1^{-/-} MEFs were washed and selected after 3 days with 250 μg/ml hygromycin B (Invitrogen) in the full complement of media. The expression of huEGFR was confirmed by flow cytometry and compared to the parental cell line or those transfected with the empty vector.

Mcl-1^{-/-} MEFs expressing huEGFR or the pLVX empty vector (Vct Ctrl) were treated with AB033-targeted Bcl-xL-ADCs, AB033 alone or MSL 109-targeted Bcl-xL-ADCs for 96 hours in DMEM containing 10% FBS. Cytotoxicity was subsequently determined using CellTiter Glo™ (Promega) and calculated as a percentage of control treated cells. For the assay, the cells were plated at 250 cells per well in 384-well tissue culture plates (Corning, Corning, N.Y.) in a total volume of 25 μL of assay media (DMEM and 10% HI FBS). The plated cells were treated with a 4-fold serial dilution of the Antibody Drug Conjugates of interest from 1 μM to 1 μM dispensed by an Echo 550 Acoustic Liquid Handler (Labcyte). Each concentration was tested in twelve replicates for the Mcl-1^{-/-} MEF huEGFR cell line and in six replicates for the Mcl-1^{-/-} MEF vector cell line. The fraction of viable cells following 96 hours of Antibody Drug Conjugate treatment at 37° C. and 5% CO₂ was determined using the CellTiter-Glo® Luminescent Cell Viability Assay according to the manufacturer's recommendations (Promega Corp., Madison, Wis.). The plates were read in a Perkin Elmer Envision using a Luminescence protocol with 0.5 sec integration time. The replicate values for each dilution point were averaged and the EC₅₀ values for the Antibody Drug Conjugates were generated by fitting the data with GraphPad Prism 5 (GraphPad Software. Inc.) to a sigmoidal curve model using linear regression, Y=((Bottom-Top)/(1+((x/K)ⁿ)))+Top, where Y is the measured response, x is the compound concentration, n is the Hill Slope and K is the EC₅₀ and Bottom and Top are the lower and higher asymptotes respectively. Visual inspection of curves was used to verify curve fit results. Mcl-1^{-/-} MEFs were obtained from David C. S. Huang of the Walter and Eliza Hall Institute of Medical Research.

### 7.2. Results

Cell viability assay results (EC₅₀ in nanomolar) for representative Examples are provided below in Table 5, below:

TABLE 5
In Vitro Cell Viability Efficacy
of Exemplary EGFR-Targeted ADC

| Appln | | huEGFR⁺ mcl-1^{-/-} MEF |
|---|---|---|
| Ex. No. | ADC Code | EC₅₀ (nM) |
| 3.1 | AB033-BS | 0.065 |
| 3.2 | AB033-DK | 0.015 |
| 3.3 | AB033-DQ | 0.055 |
| 3.4 | Ab033-DJ | 0.069 |
| 3.5 | AB033-DO | 0.5 |
| 3.6 | AB033-DP | 0.29 |
| 3.7 | AB033-HO | 2.1 |
| 3.8 | AB033-KA | 0.26 |
| 3.9 | AB033-KB | 0.2 |
| 3.10 | AB033-KT | 0.77 |
| 3.11 | AB033-KU | 1.13 |
| 3.12 | AB033-KV | 0.85 |
| 3.13 | AB033-KW | 0.51 |
| 3.14 | AB033-KZ | 52.9 |
| 3.15 | AB033-LW | 1.07 |
| 3.16 | AB033-LY | 1.3 |
| 3.17 | AB033-LZ | 1.29 |
| 3.18 | AB033-MB | 1.1 |
| 3.19 | AB033-MC | 1.21 |
| 3.20 | AB033-ME | 0.91 |
| 3.21 | AB033-MF | 0.87 |
| 3.22 | AB033-MH | 0.85 |
| 3.23 | AB033-MI | 0.85 |
| 3.24 | AB033-NJ | 0.89 |
| 3.25 | AB033-NK | 0.78 |
| 3.26 | AB033-NL | 1.04 |
| 3.27 | AB033-NM | 6.84 |
| 3.28 | AB033-NR | NT |
| 3.35 | AB033-EB | 0.15 |
| 3.39 | AB033-OG | 55 |
| 3.40 | AB033-OH | 84 |
| 3.41 | AB033-ON | 112 |
| 3.42 | AB033-OT | 62 |
| 3.43 | AB033-OP | 53 |
| 3.44 | AB033-OU | 213 |
| 3.45 | AB033-OO | 179 |
| 3.46 | AB033-OQ | 163 |
| 3.47 | AB033-OR | 75 |
| 3.48 | AB033-OS | 9.8 |
| 3.49 | AB033-PA | 66 |
| 3.50 | AB033-QL | >1000 |
| 3.51 | AB033-QM | >1000 |
| 3.52 | AB033-QN | >1000 |
| 3.53 | AB033-QT | >1000 |
| 3.54 | AB033-RF | 351 |
| 3.55 | AB033-RG | 122 |
| 3.56 | AB033-SF | 111 |
| 3.57 | AB033-SR | 3.2 |
| 3.58 | AB033-YZ | 16 |
| 3.59 | AB033-QR | >1000 |
| 3.60 | AB033-SE | 46 |
| 3.61 | AB033-UH | 1.8 |
| 3.62 | AB033-UI | 32 |
| 3.63 | AB033-US | 440 |
| 3.64 | AB033-UY | 611 |
| 3.65 | AB033-UX | 810 |
| 3.66 | AB033-WZ | 542 |
| 3.67 | AB033-XO | 444 |
| 3.68 | AB033-XW | NT |
| 3.69 | AB033-YG | <1 |
| 3.70 | AB033-ZT | 25 |
| 3.71 | AB033-AAN | 16 |
| 3.72 | AB033-AAO | 8.1 |
| 3.73 | AB033-AAP | 24 |
| 3.74 | AB033-ZZ | 11 |

NT = not tested,
NV = not valid

Cell viability assay results (EC₅₀ in nanomolar) for representative Examples 3.8, 3.9, 3.19, 3.64, 3.65, 3.66, 3.67. 3.70, 3.72 and 3.74 against the Mcl-1^{-/-} MEF vector cell line are 53 nM, 67 nM, 32 nM, >1,000 nM, >1,000 nM, 621 nM, >1,000 nM >250 nM, 831 nM and 553 nM, respectively.

### Example 8. Exemplary EGFR-Targeted ADCs Inhibit the Growth of Tumors In Vivo

The ability of certain exemplary EGFR-targeted ADCs to inhibit the growth of tumor cells in vivo in mice was demonstrated in a xenograft model with tumors derived from NCI-H1650 cells, a human non small cell lung cancer (NSCLC) cell line.

### 8.1. Method

The NSCLC cell line NCI-H1650 was purchased from the American Type Culture Collection (ATCC, Manassas. Va.). The cells were cultured as monolayers in RPMI 1640 culture medium (Invitrogen, Carlsbad, Calif.) that was supplemented with Fetal Bovine Serum (FBS, Hyclone, Logan, Utah). Five million viable cells NCI-H1650 cells were inoculated subcutaneously into the right flank of immune deficient female SCID/bg mice (Charles River Laboratories, Wilmington, Mass.). The injection volume was 0.2 ml and composed of a 1:1 mixture of S-MEM and Matrigel (BD, Franklin Lakes, N.J.). Tumors were size matched at approximately 200 mm³. Antibodies and conjugates were formulated in phosphate buffered saline (PBS) and injected intraperitoneally. Injection volume did not exceed 400 μl. Therapy began within 24 hours after size matching of the tumors. Mice weighed approximately 25 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Eight to ten mice were housed per cage. Food and water were available ad libitum. Mice were acclimated to the animal facilities for a period of at least one week prior to commencement of experiments. Animals were tested in the light phase of a 12-hour light: 12-hour dark schedule (lights on at 06:00 hours). All experiments were conducted in compliance with AbbVie's Institutional Animal Care and Use Committee and the National Institutes of Health Guide for Care and Use of Laboratory Animals guidelines in a facility accredited by the Association for the Assessment and Accreditation of Laboratory Animal Care.

The EGFR-targeted ADCs 3.6, 3.10, 3.14, 3.8, 3.9, 3.13, 3.61, 3.62, 3.63, 3.64 and 3.65 were prepared according to procedures in Example 3 (Synthesis of exemplary ADCs), Table 1. A conjugate of synthon H (see Example 2.32) and the CMV targeting antibody MSL109 (MSL109-H) was used as a passive targeting control. This conjugate is hereafter also referred to as ‘non-targeting’ ADC because the carrier antibody does not recognize a tumor associated antigen. MSL 109 is described in Drobyski et al., 1991, Transplantation, 51:1190-1196 and U.S. Pat. No. 5,750,106. An antibody that targets tetanus toxoid (antibody AB095) was used as a control for the effect of administering IgG. See Larrick et al., 1992, Immunological Reviews, 69-85. The efficacy of inhibition of H1650 xenograft growth with EGFR-targeted ADCs is illustrated by Table 6, 7 and 8, below. The tumor growth inhibition by EGFR-targeting control antibody and ‘non-targeting’ ADCs is described in Table 9. Treatment was initiated at earliest 11 days (Table 6) or at latest 15 days (Table 8) post inoculation of tumor cells. The approximate tumor size at onset of treatment was between 210 mm³ and 230 mm³. All conjugates and antibodies were given intraperitoneally. The doses and regimens of treatment arc specified in the tables.

### 8.2. Results

### 8.2.1. Parameters of Efficacy and Statitical Analysis

The efficacy of inhibition of H1650 xenografts growth with EGFR-targeted ADCs is illustrated by Table 6, Table 7, and Table 8, below. In the tables, to refer to efficacy, parameters of amplitude (TGIₘₐₓ) and durability (TGD) of therapeutic response are used.

TGIₘₐₓ is the maximum tumor growth inhibition during the experiment. Tumor growth inhibition is calculated by 100*(1−Tᵥ/Cᵥ) where Tᵥ and Cᵥ are the mean tumor volumes of the treated and control groups, respectively.

TGD or tumor growth delay is the extended time of a treated tumor needed to reach a volume of 1 cm³ relative to the control group. TGD is calculated by 100*(Tₜ/Cₜ−1) where Tₜ and Cₜ are the median time periods to reach 1 cm³ of the treated and control groups, respectively.

Distribution of the response amplitude in a specific group is given by the frequency of complete responders (CR), partial responders (PR), and overall responders (OR). CR is the percentage of mice within a group with a tumor burden of 25 mm³ for at least three measurements. PR is the percentage of mice within a group with a tumor burden larger than 25 mm³ but less than one-half of the volume at onset of treatment for at least three measurements. OR is the sum of CR and PR.

The 2-tailed Student's test and Kaplan-Meier log-rank test were used to determine significance of the difference in TGIₘₐₓ and TGD, respectively.

### 8.2.2. Efficacy of EGFR-Targeting Bcl-xLi ADCs In Vivo

A single dose of 10 mg/kg of the EGFR targeting Bcl-xL inhibitory ADC (also referred to herein as Bcl-xLi ADC) consistently inhibited tumor growth. The most active conjugate. AB033-KZ inhibited tumor growth by 96%. The durability of the response was evidenced by a TGD of 233%. This conjugate also induced 86% overall response rates. The lowest activity observed was following treatment with AB033-KB. This conjugate inhibited tumor growth by 62% and caused a tumor growth delay of 40%. AB033-KB did neither induce complete nor partial responses. The efficacy of the EGFR-targeting BclxL inhibitory conjugates is unlikely due to the activity of the carrier antibody or to activity from passive targeting. Historical controls (Table 9) show that the minimum total amount of AB033 necessary to have equivalent efficacy of AB033-KB is approximately 18 mg/kg given as 6 doses of 3 mg/kg with an interval of 4 days. The non-targeting ADC, MSL 109-H could not equal the efficacy of AB033-KB even when a total amount of 60 mg/kg was administered. Neither treatment with AB033 nor reatment with MSL109-H induced complete or partial responses.

TABLE 6
Inhibition of H1650 xenograft tumor growth after treatment with a
single dose of EGFR-targeting Bcl-xLi ADCs

| | | | Growth Inhibition | | Response Frequency | | |
|---|---|---|---|---|---|---|---|
| | | Dose^{[a]}/route/ | TGIₘₐₓ | TGD | CR | PR | OR |
| Ex. No. | Treatment | regimen | (%) | (%) | (%) | (%) | (%) |
| IgG1 | AB095^{**,†} | 10/IP/QDx1 | 0 | 0 | 0 | 0 | 0 |
| mAb | | | | | | | |
| Non- | MSL109-H | 10/IP/QDx1 | 20* | 7 | 0 | 0 | 0 |
| targeting | | | | | | | |
| ADC | | | | | | | |
| 3.6 | AB033-HO | 10/IP/QDxl | 89* | 160* | 13 | 75 | 88 |

^{[a]}dose is given in mg/kg/day
**IgG1 mAb
^{†}Non-targeting antibody
*= P < 0.05 as compared to control treatment (AB095)

TABLE 7
Inhibition of H1650 xenograft tumor growth after treatment with
a single dose of EGFR-targeting Bcl-xLi ADCs

| | | Dose^{[a]}/ | Growth Inhibition | | Response Frequency | | |
|---|---|---|---|---|---|---|---|
| Ex. | | route/ | TGIₘₐₓ | TGD | CR | PR | OR |
| No. | Treatment | regimen | (%) | (%) | (%) | (%) | (%) |
| IgG1 | AB095^{**,†} | 10/IP/ | 0 | 0 | 0 | 0 | 0 |
| mAb | | QDx1 | | | | | |
| 3.10 | AB033-KT | 10/IP/ | 93* | 137* | 0 | 75 | 75 |
| | | QDx1 | | | | | |
| 3.14 | AB033-KZ | 10/IP/ | 96* | 233* | 71 | 14 | 86 |
| | | QDx1 | | | | | |
| 3.8 | AB033-KA | 10/IP/ | 78* | 47* | 0 | 0 | 0 |
| | | QDx.1 | | | | | |
| 3.9 | AB033-KB | 10/IP/ | 62* | 40* | 0 | 0 | 0 |
| | | QDx.1 | | | | | |
| 3.13 | AB033-KW | 10/IP/ | 87* | 87* | 0 | 25 | 25 |
| | | QDx.1 | | | | | |

^{[a]}dose is given in mg/kg/day
**IgG1 mAb
^{†}Non-targeting antibody
*= P < 0.05 as compared to control treatment (AB095)

TABLE 8
Inhibition of H1650 xenograft tumor growth after treatment
with a single dose of EGFR-targeting Bcl-xLi ADC

| | | Dose^{[a]}/ | Growth Inhibition | | Response Frequency | | |
|---|---|---|---|---|---|---|---|
| Ex. | | route/ | TGIₘₐₓ | TGD | CR | PR | OR |
| No. | Treatment | regimen | (%) | (%) | (%) | (%) | (%) |
| | AB095^{**,†} | 10/IP/ | 0 | 0 | 0 | 0 | 0 |
| | | QDx1 | | | | | |
| 3.67 | AB033-UX | 10/IP/ | 82* | 89* | 0 | 25 | 25 |
| | | QDx1 | | | | | |
| 3.66 | AB033-UY | 10/IP/ | 81* | 84* | 0 | 25 | 25 |
| | | QDx1 | | | | | |
| 3.65 | AB033-US | 10/IP/ | 70* | 74* | 13 | 13 | 25 |
| | | QDx1 | | | | | |
| 3.64 | AB033-UI | 10/IP/ | 75* | 74* | 0 | 13 | 13 |
| | | QDx1 | | | | | |
| 3.63 | AB033-UH | 10/IP/ | 62* | 53* | 0 | 0 | 0 |
| | | QDx1 | | | | | |

** IgG1 mAb
^{†}Non-targeting antibody
^{[a]}dose is given in mg/kg/day
*= p < 0.05 as compared to control treatment (AB095)

TABLE 9
Inhibition of H1650 xenograft tumor growth after treatment with EGFR-
targeting antibody, AB033 and ‘non-targeting, ADC, MSL109-H

| | | Growth Inhibition | | Response Frequency | | |
|---|---|---|---|---|---|---|
| | Dose^{[a]}/route/ | TGIₘₐₓ | TGD | CR | PR | OR |
| Treatment | regimen | (%) | (%) | (%) | (%) | (%) |
| AB033 | 3/IP/Q4Dx6 | 17* | 0 | 0 | 0 | 0 |
| AB033 | 3/IP/Q4Dx6 | 54* | 44* | 0 | 0 | 0 |
| AB033 | 10/IP/Q4Dx6 | 62* | 56* | 0 | 0 | 0 |
| MSL,109†-H | 3/IP/Q4Dx6 | 18* | 0 | 0 | 0 | 0 |
| MSL109†-H | 10/IP/ Q4Dx6 | 43* | 20* | 0 | 0 | 0 |
| MSL109†-H | 10/1P/Q4Dx6 | 8 | 0 | 0 | 0 | 0 |

^{[a]}dose is given in mg/kg/day
^{†}Non-targeting antibody
*= P < 0.05 as compared to control treatment (AB095)

### Example 9. Bcl-xLi Antibody-Drug Conjugates Mitigate Systemic Toxicity

### 9.1. Circumvention of Thrombocytopenia

Administration of Bcl-xLi ADCs as antibody drug conjugate can possibly circumvent the systemic toxicity of the small molecule via selective targeting of the tumor. In this manner, the ADC can bypass systemic toxicity and allow tumor-specific efficacy via two possible mechanisms. For ADCs with a cell membrane permeating Bcl-xL inhibitor, the binding to the carrier antibody can limit systemic exposure to the small molecule.

### 9.1.1. Method & Results

The influence of two Bcl-xLi ADCs on the number of circulating platelets in mice was tested following a single intraperitoneal injection (the inhibitory ADCs are comprised of anti-EGFR antibody AB033 and control synthons H and I (Examples 2.32 and 2.33) are designated AB033-H and AB033-I). The anti-tetanus toxoid antibody AB095 was used as a negative control. Navitoclax (ABT-263, a dual Bcl-2 and Bcl-xL inhibitor), A-1331852 (selective Bcl-xL inhibitor, Leverson et al., 2015, Sci. Transl. Med. 7:279ra40) and the unconjugated Bcl-xL inhibitor (Example 2.32.24, positive control) caused thrombocytopenia which was maximal at 6 hours following injection of the compounds. A dose of 0.61 mg/kg, which is the equivalent amount of Bcl-xL inhibitor found in Bcl-xLi ADC at 30 mg/kg, decreased the platelet number 100-fold from a normal count of approximately 6*10⁵/mm³ to 6*10³/mm³.

In contrast, none of the Bcl-xLi ADCs caused a meaningful reduction of the platelets 6 hours after administration (Table 10) or at any time point during an observation period of 14 days. The latter observation renders induction of thrombocytopenia caused by slow release of the inhibitor from the ADCs is unlikely.

TABLE 10
Influence of Bcl-xLi ADCs with ceil permeating Bcl-xL
inhibitors on the number of circulating platelets

| | | Lowest | Time to |
|---|---|---|---|
| | Dose | thrombocyte | lowest count |
| Compound | (mg/kg) | count | (hours) |
| none | | 594 | 0 |
| AB095 | 30 | 539 | 6 |
| ABT-263 | 100 | 10 | 6 |
| Example 2.32.24 | 0.61 | 6 | 6 |
| A-1331852 | 25 | 9 | 6 |
| AB033-I | 30 | 335 | 72 |
| AB033-I | 10 | 567 | 72 |
| AB033-H | 30 | 521 | 72 |

Platelet count is presented as 1/10³ of the platelet#/mm³

While various specific embodiments have been illustrated and described, it will be appreciated that various changes can be made without departing from the spirit and scope of the disclosure.

What is claimed is:

1.-68. (canceled)

69. A method of making an ADC, comprising contacting a synthon according to structural formula D-L-R^{x}, or a pharmaceutically acceptable salt thereof, wherein:
D is a Bcl-xL inhibitor according to structural formula (IIa) or (IIb): 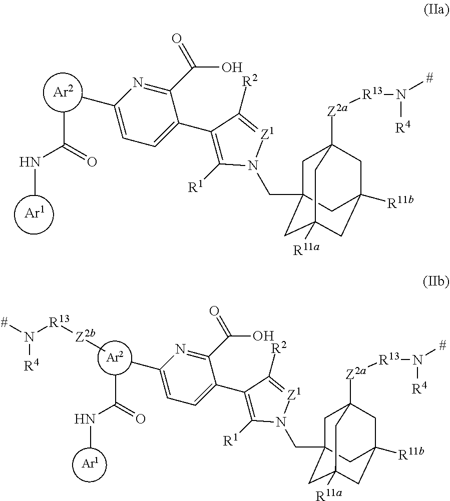
or pharmaceutically acceptable salts thereof, wherein:
Ar¹ is selected from 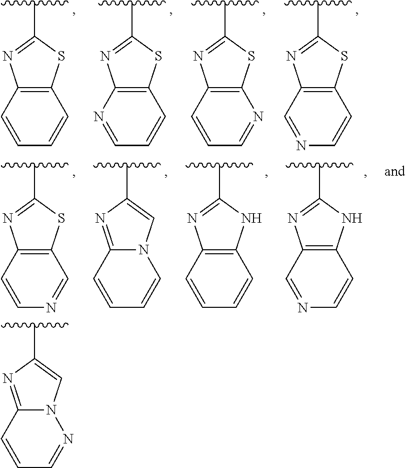
and is optionally substituted with one or more substituents independently selected from halo, hydroxy, nitro, lower alkyl, lower heteroalkyl, alkoxy, amino, cyano and halomethyl;
Ar² is selected from 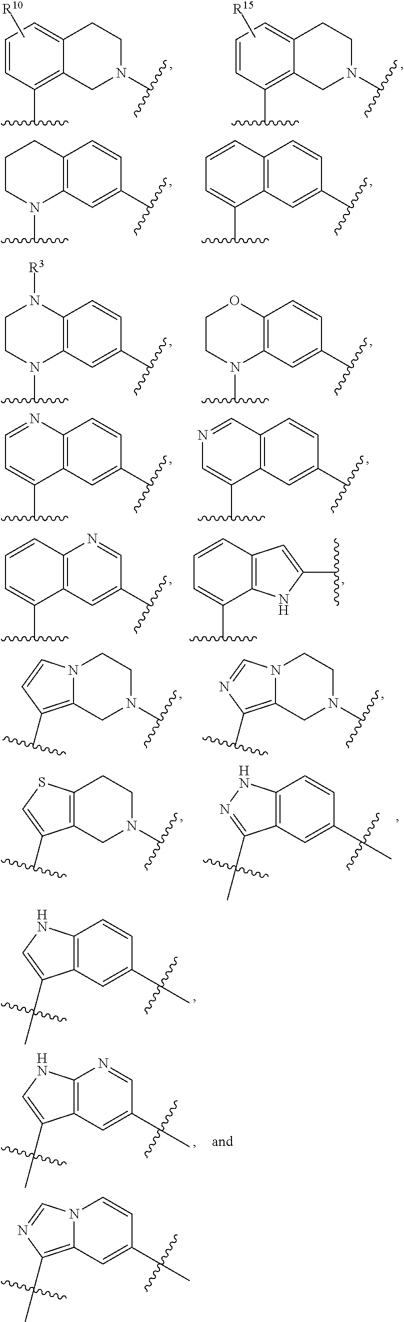
and is optionally substituted with one or more substituents independently selected from halo, hydroxy, nitro, lower alkyl, lower heteroalkyl, alkoxy, amino, cyano and halomethyl, wherein the #-N(R⁴)—R¹³—Z^{2b}— substituent of formula (IIb) is attached to Ar² at any Ar² atom capable of being substituted;
Z¹ is selected from N, CH, C-halo and C—CN;
Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O);
R¹ is selected from hydrogen, methyl, halo, halomethyl, ethyl and cyano;
R² is selected from hydrogen, methyl, halo, halomethyl and cyano;
R³ is selected from hydrogen, lower alkyl and lower heteroalkyl;
R⁴ is selected from hydrogen, lower alkyl, monocyclic cycloalkyl, monocyclic heterocyclyl, and lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, and lower heteroalkyl are optionally substituted with one or more halo, cyano, hydroxy, alkoxy, monocyclic cycloalkyl, monocyclic heterocyclyl, C(O)NR^{6a}R^{6b}, S(O₂)NR^{6a}R^{6b}, NHC(O)CHR^{6a}R^{6b}, NHS(O)CHR^{6a}R^{6b}, NHS(O₂)CHR^{6a}R^{6b}, S(O₂)CHR^{6a}R^{6b} or S(O₂)NH₂ groups;
R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;
R¹⁰ is selected from cyano, OR¹⁴, SR¹⁴, SOR¹⁴, SO₂R¹⁴, SO₂NR^{14a}R^{14b}, NR^{14a}R^{14b}, NHC(O)R¹⁴ and NHSO₂R¹⁴;
R^{11a} and R^{11b} are each, independently of one another, selected from hydrogen, halo, methyl, ethyl, halomethyl, hydroxyl, methoxy, CN, and SCH₃;
R¹² is selected from hydrogen, halo, cyano, lower alkyl, lower heteroalkyl, cycloalkyl, and heterocyclyl, wherein the alkyl, heteroalkyl, cycloalkyl, and heterocyclyl are optionally substituted with one or more halo, cyano, alkoxy, monocyclic cycloalkyl, monocyclic heterocyclyl, NHC(O)CHR^{6a}R^{6b}, NHS(O)CHR^{6a}R^{6b}, NHS(O₂)CHR^{6a}R^{6b} or S(O₂CHR^{6a}R^{6b} groups;
R¹³ is selected from a bond, optionally substituted lower alkylene, optionally substituted lower heteroalkylene, optionally substituted cycloalkyl or optionally substituted heterocyclyl;
R¹⁴ is selected from hydrogen, optionally substituted lower alkyl and optionally substituted lower heteroalkyl;
R^{14a} and R^{14b} are each, independently of one another, selected from hydrogen, optionally substituted lower alkyl, and optionally substituted lower heteroalkyl, or are taken together with the nitrogen atom to which they are bonded to form an optionally substituted monocyclic cycloalkyl or monocyclic heterocyclyl ring;
R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃;
where # represents the point of attachment to a linker L;
L is a linker; and
R^{x} is a moiety comprising a functional group capable of covalently linking the synthon to an antibody;
with an antibody under conditions in which the synthon covalently links to the antibody.

70.-87. (canceled)

88. The method of claim 69, or a pharmaceutically acceptable salt thereof, in which the linker is cleavable by a lysosomal enzyme.

89. The method of claim 88, or a pharmaceutically acceptable salt thereof, in which the lysosomal enzyme is Cathepsin B.

90. The method of claim 69 in which the linker comprises a segment according to structural formula (VIIa), (VIIb), or (VIIc): 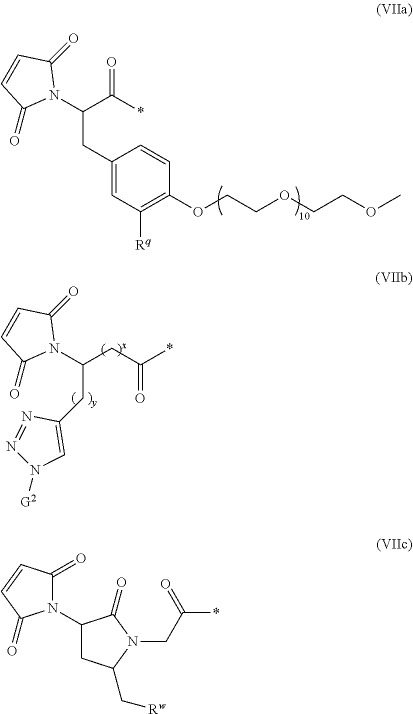
or salts thereof, wherein:
R^{q} is H or —O—(CH₂CH₂O)₁₁—CH₃;
x is 0 or 1;
y is 0 or 1;
G² is —CH₂CH₂CH₂SO₃H or —CH₂CH₂O—(CH₂CH₂O)₁₁—CH₃;
R^{w} is —O—CH₂CH₂SO₃H or —NH(CO)—CH₂CH₂O—(CH₂CH₂O)₁₂—CH₃;
* represents the point of attachment to the remainder of the linker.

91. The method of claim 69 in which the linker comprises a segment according to structural formula (IVa), (IVb), (IVc), or (Vd): 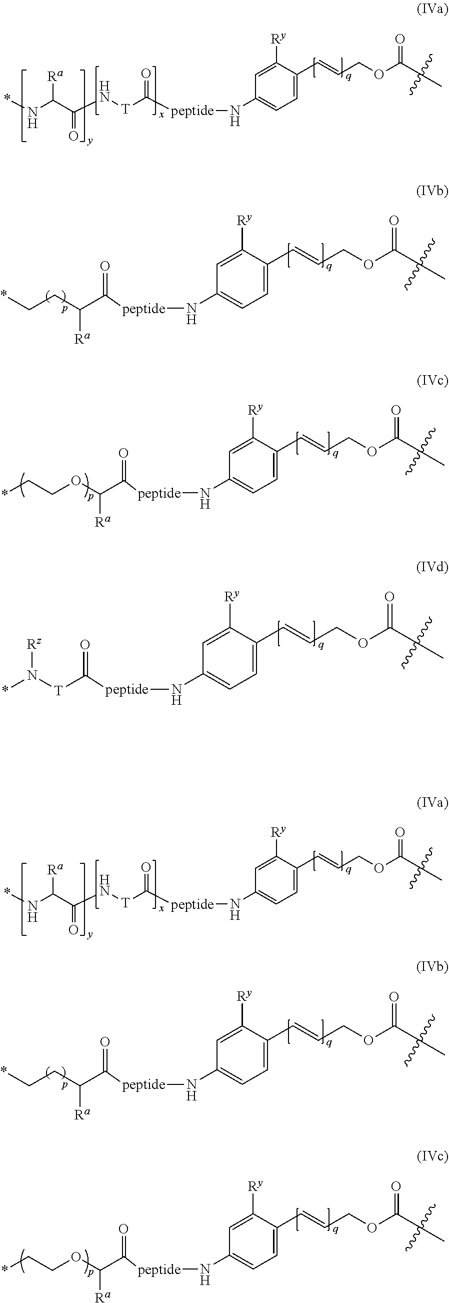
or a pharmaceutically acceptable salt thereof, wherein:
peptide represents a peptide (illustrated N→C, wherein peptide includes the amino and carboxy “termini”) a cleavable by a lysosomal enzyme;
T represents a polymer comprising one or more ethylene glycol units or an alkylene chain, or combinations thereof;
R^{a} is selected from hydrogen, alkyl, sulfonate and methyl sulfonate;
R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂;
R^{z} is C₁₋₄ alkyl-(O)ᵣ(C₁₋₄ alkylene)ₛ-G²;
G¹ is SO₃H, CO₂H, PEG 4-32, or sugar moiety;
G² is SO₃H, CO₂H, or PEG 4-32 moiety;
r is 0 or 1;
p is an integer ranging from 0 to 5;
q is 0 or 1;
x is 0 or 1;
y is 0 or 1; [symbol]
represents a point of attachment of the linker to the Bcl-xL inhibitor; and
* represents the point of attachment to the remainder of the linker.

92. The method of claim 91, or a pharmaceutically acceptable salt thereof, in which peptide is selected from the group consisting of Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, and salts thereof.

93. The method of claim 88, or a pharmaceutically acceptable salt thereof, in which the lysosomal enzyme is β-glucuronidase.

94. The method of claim 93 in which the linker comprises a segment according to structural formula (Va), (Vb), (Vc), or (Vd): 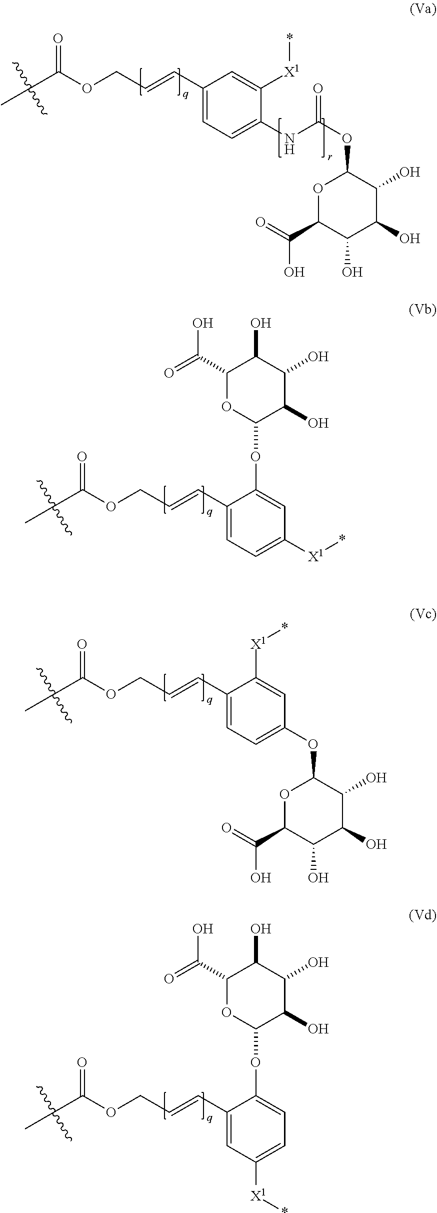
or a pharmaceutically acceptable salt thereof, wherein:
q is 0 or 1;
r is 0 or 1;
X¹ is CH₂, O or NH; [symbol]
represents the point of attachment of the linker to the drug; and
represents the point of attachment to the remainder of the linker.

95. The method of claim 69, or a pharmaceutically acceptable salt thereof, in which the linker comprises a polyethylene glycol segment having from 1 to 6 ethylene glycol units.

96. The method of claim 69, or a pharmaceutically acceptable salt thereof, in which linker L is selected from IVa or IVb and salts thereof.

97. The method of claim 69, or a pharmaceutically acceptable salt thereof, in which R^{x} comprises a functional group capable of linking the synthon to an amino group on an antibody.

98. The method of claim 97, or a pharmaceutically acceptable salt thereof, in which R^{x} comprises an NHS-ester or an isothiocyanate.

99. The method of claim 88, or a pharmaceutically acceptable salt thereof, in which R^{x} comprises a functional group capable of linking the synthon to a sulfhydryl group on an antibody.

100. The method of claim 99, or a pharmaceutically acceptable salt thereof, in which R^{x} comprises a haloacetyl or a maleimide.

101. The method of claim 69, or a pharmaceutically acceptable salt thereof, in which:
L is selected from (IVa), (IVb), (IVc), (IVd), and salts thereof; and
R^{x} comprises a functional group selected from the group consisting of NHS-ester, isothiocyanate, haloacetyl and maleimide.
